(19) United States
(12) Patent Application Publication
(10) Pub. No.: US 2006/0025474 A1
(43) Pub. Date: Feb. 2, 2006

(54) Bisphenyl compounds useful as vitamin D3 receptor agonists

(76) Inventors: David Wallace, San Diego, CA (US); Thomas Arrhenius, Del Mar, CA (US); Anna Russell, San Diego, CA (US); Dingguo Liu, San Marcos, CA (US); Amy Xing, San Diego, CA (US); Sovouthy Tith, Phoenix, AZ (US); Zheng Hou, San Diego, CA (US); Tadakatsu Takahashi, Shizuoka (JP); Yoshiyuki Ono, Shizuoka (JP); Hirotaka Kashiwagi, Shizuoka (JP); Kazuki Shimizu, Shizuoka (JP); Hitoshi Ikura, Kanagawa (JP)

Correspondence Address: FISH & RICHARDSON, PC, P.O. BOX 1022, MINNEAPOLIS, MN, 55440-1022, US

(21) Appl. No.: 11/076,584

(22) Filed: Mar. 8, 2005

Related U.S. Application Data
(60) Provisional application No. 60/551,193, filed on Mar. 8, 2004.

(51) Int. Cl. A61K 31/366; A61K 31/365; A61K 31/192

(52) U.S. Cl. 514/460; 514/568; 514/474; 549/295; 562/466

(57) ABSTRACT

The present invention discloses bisphenyl compounds of the formula: 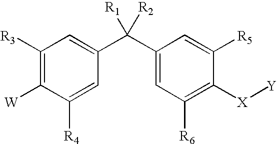 wherein R1, R2, R3, R4, R5, R6, X, Y, W are defined herein after. These compounds are useful as pharmaceuticals.

### RELATED APPLICATIONS

Priority is claimed herein under 35 U.S.C. §119(e) to U.S. provisional patent application No. 60/551,193, filed Mar. 08, 2004, entitled BISPHENYL COMPOUNDS USEFUL AS VITAMIN D3 RECEPTOR AGONISTS. The disclosure of the above-referenced application is incorporated by reference herein in its entirety.

### FIELD OF THE INVENTION

This invention relates to novel bisphenyl compounds and their pharmaceutically acceptable salts, pharmaceutical compositions containing said such bisphenyl compounds and the use thereof.

### BACKGROUND OF THE INVENTION

It is well-known that 1α,25-Dihydroxyvitamin D3 has various physiological activities and is widely used as various pharmaceuticals. For example, the pharmaceutical agent containing 1α,25-Dihydroxyvitamin D3 as active ingredient marketed as ROCALTROL™ and used for the treatment of psoriasis and secondary hyperparathyroidism. However, it is also well-known that 1α,25-Dihydroxyvitamin D3 has a serious side effect elevating serum calcium level and sometimes it leads to hypercalcemia. Because of this side effect, there are some limitations in administering 1α,25-Dihydroxyvitamin D3, such as, for example, limitation of maximum administration dosages or limitation of patients to be treated, and therefore, the benefit of the various physiological activities of the compound for treating diseases cannot be fully redlined.

To overcome this problem, various derivatives of vitamin D3 have been synthesized, and recently, mimic compounds of the vitamin D3 not having a secosteroidal structure (VD3 mimics) were suggested. For example, International Publication No. WO00/10958 and corresponding U.S. Pat. No. 6,218,430 B1 disclose bisphenyl compounds useful as VD3 mimics, however, no compound has been launched or tested in clinical trials. Accordingly, there is a demand for a vitamin D3 derivative with stronger physiological activities and weaker calcemic effect than VD3 mimics and compounds previously described.

### SUMMARY OF THE INVENTION

The present invention relates to bisphenyl compounds useful as modulators of the vitamin D receptor activity or their synthetic intermediates, represented by formula I: 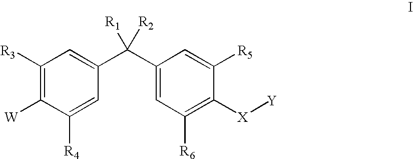
wherein

X is an optionally substituted methylene, an optionally substituted ethylene, an optionally substituted vinylene, an ethynylene, —S(O)n, —NH—, or —O—;

n is an integer of 0 to 2;

Y is COOR8, CON(R9)R10, S(O)mR11 or a substituent represented by following formula:

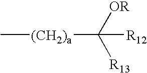

R8 and R11 are each independently selected from an optionally substituted C1-10 alkyl group or an optionally substituted C3-10 cycloalkyl group;

R9 and R10 are each independently selected from a hydrogen atom, an optionally substituted C1-10 alkyl group or an optionally substituted C3-10 cycloalkyl group;

m is an integer of 0 to2;

a is an integer of 0 to 3;

R is a hydrogen atom or a protecting group for a hydroxyl group;

R12 and R13 are each independently selected from the group consisting of a hydrogen atom, an optionally substituted C1-10 alkyl group, an optionally substituted C3-10 cycloalkyl group, an optionally substituted C2-10 alkenyl group, an optionally substituted C2-10 alkynyl group, or R12 and R13 may together form an-optionally substituted C3-C12 cycloalkyl group or an optionally substituted 3-12 membered heterocyclic group;

W is a hydroxyl group, a carboxyl group, a trifluoromethanesulfonyloxy group or a substituent represented by following formula:

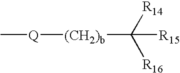

Q is —O—, —S—, —NH—, an optionally substituted methylene, an optionally substituted ethylene, an optionally substituted vinylene, an ethynylen, —(CH2)ₖ-NHC(═O)—, —(CH2)ₖ-C(═O)NH—, —(CH2)ₖ-NHC(═O)NH—, —O—(CH2)ₖ-NHC(═O)—, —O—(CH2)ₖ-C(═O)NH—, —O—(CH2)ₖ-NHC(═O)NH— or —(CH2)ₖ-SO₂—;

b is an integer of 0 to 10;

k is an integer of 0 to 2;

R14 is a hydrogen atom, a hydroxyl group, an optionally substituted carboxyl group, an optionally substituted carbamoyl group, an optionally substituted C1-6 alkyl group, an optionally substituted C3-C12 cycloalkyl group, an optionally substituted C1-6 alkenyl group, an optionally substituted C1-6 alkynyl group, an optionally substituted C6-C12 aryl group, an optionally substituted (C6-C12)aryl-(C -4)alkyl group, —OR17 or —N(R18)R19;

R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group, or R15 and R16 may together form ═O;

R17 is selected from an optionally substituted C1-6 alkyl group or an optionally substituted C3-C6 cycloalkyl group;

R18 and R19 are each independently selected from the group consisting of a hydrogen atom, an optionally substituted C1-6 alkyl group, an optionally substituted C3-C6 cycloalkyl group, or R18 and R19 may together form an optionally substituted C3-C12 cycloalkyl group or an optionally substituted 3-12 membered heterocyclic group;

or one of (R15 and R17), (R16 and R17), (R15 and R18), (R16 and R18), (R15 and R19) or (R16 and R19) may together form a 3-12 membered cyclic ring which is selected from the group consisting of an amidine ring, an amine ring, an ether ring, a lactam ring, a lactone ring, an acetal ring, a hemiacetal ring, a carbonate ring, a carbamate ring, an urea ring, combinations thereof;

R1 and R2 are each independently selected from the group consisting of a C1-6 alkyl group optionally substituted with a halogen atom(s), a C3-6 cycloalkyl group optionally substituted with a halogen atom(s), a C2-6 alkenyl group optionally substituted with a halogen atom(s), a C2-6 alkynyl group optionally substituted with a halogen atom(s), or R1 and R2 may together form a C3-8 cycloalkyl group optionally substituted with a halogen atom(s), a C3-8 cycloalkenyl group optionally substituted with a halogen atom(s) or a C3-8 cycloalkylidene group optionally substituted with a halogen atom(s);

R3, R4, R5 and R6 are each independently selected from the group consisting of a hydrogen atom, a halogen atom, a C1-6 alkyl group optionally substituted with a halogen atom(s), a C3-6 cycloalkyl group optionally substituted with a halogen atom(s);

When X is —S(O)n-, —NH— or —O—, Q is selected from the group consisting of a methylene which may be substituted an C1-4 alkyl group, an ethylene, a vinylene, an ethynylene, —(CH2)ₖ-NHC(═O)—, —(CH2)ₖ-C(═O)NH—, —(CH2)ₖ-NHC(═O)NH—, —(CH2)ₖ-SO₂—;

When Q is —O—, —S—, —NH—, —O—(CH2)ₖ-NHC(═O)—, —O—(CH2)ₖ-C(═O)NH— or —O—(CH2)ₖ-NHC(═O)NH—, X is selected from the group consisting of an optionally substituted methylene, an optionally substituted ethylene, an optionally substituted vinylene, an ethynylene; and pharmaceutically acceptable salts and prodrugs thereof.

### DETAILED DESCRIPTION OF THE INVENTION

Definitions

“Optionally substituted” or “which may be substituted” means that the compounds of the present invention can optionally have one or more substituent(s) in that position. In the case having two or more substituents, each substituents can be same or different.

A “halogen atom” according to the invention is a fluorine atom, a chlorine atom, a bromine atom or an iodine atom.

A “C1-10 alkyl group” according to the invention is a linear or branched alkyl group of 1 to 10 carbons, such as a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a hexyl group, an isohexyl group, a heptyl group, an octyl group, a nonyl group, a decyl group or the like.

A “C1-6 alkyl group” according to the invention is a linear or branched alkyl group of 1 to 6 carbons, such as a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a hexyl group, an isohexyl group or the like.

A “C1-4 alkyl group” according to the invention is a linear or branched alkyl group of 1 to 3 carbons, such as a methyl group, an ethyl group, a propyl group, an isopropyl group, a butyl group, an isobutyl group, a sec-butyl group, a tert-butyl group or the like.

A “C1-3 alkyl group” according to the invention is a linear or branched alkyl group of 1 to 3 carbons, such as a methyl group, an ethyl group, a propyl group, an isopropyl group or the like.

A “C3-12 cycloalkyl group” according to the invention is a cyclic saturated hydrocarbon group of 3 to 12 carbons, such as a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group or the like.

A “C3-10 cycloalkyl group” according to the invention is a cyclic saturated hydrocarbon group of 3 to 10 carbons, such as a cyclopropyl group, a cyclobutyl group, a cyclopentyl group, a cyclohexyl group, a cycloheptyl group, a cyclooctyl group, a cyclononyl group, a cyclodecyl group or the like.

A “C3-8 cycloalkyl group” according to the invention is a cyclic saturated hydrocarbon group of 3 to 8 carbons, such as a cyclopropyl group, a cyclobutyl group, a cyclopentyl group, a cyclohexyl group, a cycloheptyl group, a cyclooctyl group or the like.

A “C3-6 cycloalkyl group” according to the invention is a cyclic saturated hydrocarbon group of 3 to 6 carbons, such as a cyclopropyl group, a cyclobutyl group, a cyclopentyl group, a cyclohexyl group or the like.

A “C2-10 alkenyl group” according to the invention is a linear, branched or cyclic alkenyl group of 2 to 10 carbons, such as a vinyl group, an allyl group, an isopropenyl group, a 2-butenyl group, a 3-butenyl group, a 2-pentenyl group, a 3-pentenyl group, a 4-pentenyl group, a 1-cyclopentenyl group, a 2-hexenyl group, a 3-hexenyl group, a 4-hexenyl group, a 5-hexenyl group, a 1-cyclohexenyl group or the like.

A “C2-6 alkenyl group” according to the invention is a linear, branched or cyclic alkenyl group of 2 to 6 carbons, such as a vinyl group, an allyl group, an isopropenyl group, a 2-butenyl group, a 3-butenyl group, a 2-pentenyl group, a 3-pentenyl group, a 4-pentenyl group, a 1-cyclopentenyl group, a 2-hexenyl group, a 3-hexenyl group, a 4-hexenyl group, a 5-hexenyl group, a 1-cyclohexenyl group or the like.

A “C3-8 cycloalkenyl group” according to the invention is a cyclic alkenyl group of 3 to 8 carbons, such as a 1-cyclopentenyl group, a 1-cyclohexenyl group, 2-cyclohexenyl group or the like.

A “C2-10 alkynyl group” according to the invention is a linear or branched alkynyl group of 2 to 10 carbons, such as an ethynyl group, a 2-propynyl group, a 2-butynyl group, a 3-butynyl group, a 2-pentynyl group, a 3-pentynyl group, a 4-pentynyl group, a 2-hexynyl group, a 3-hexynyl group, a 4-hexynyl group, a 5-hexynyl group or the like.

A “C2-6 alkynyl group” according to the invention is a linear or branched alkynyl group of 2 to 6 carbons, such as an ethynyl group, a 2-propynyl group, a 2-butynyl group, a 3-butynyl group, a 2-pentynyl group, a 3-pentynyl group, a 4-pentynyl group, a 2-hexynyl group, a 3-hexynyl group, a 4-hexynyl group, a 5-hexynyl group or the like.

A “C6-12 aryl group” according to the invention is an aryl group of 6 to 12 carbons, such as a phenyl group, a naphthyl group or the like.

A “(C6-12)aryl-(C1-4)alkyl group” according to the invention is an alkyl group of 1 to 4 carbons with an aryl group of 6 to,12 carbons, such as a benzyl group, a phenethyl group, a 3-phenyl-propyl group, a 4-phenyl-butyl group, a naphthalen-1-yl-methyl group, a naphthalen-2-yl-methyl group or the like.

A “3-12 membered heterocyclic group” according to the invention is a mono- and polycyclic group having 3 to 12 membered ring(s) wherein the ring(s) contains at least one heteroatom. Suitable heteroatoms include oxygen, nitrogen, sulfur, phosphorus and boron. Heterocyclic group may be attached at a carbon atom or heteroatom. Heterocyclic groups include an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group, an isoquinoline group, a cinnoline group, a phthalazine group, a quinazoline group, a quinoxaline group or the like.

A “C1-8 alkoxy group” according to the invention is an oxygen group which is substituted with a linear or branched alkyl group, alkenyl group, alkynyl group, aralkyl group or aryl group of 1 to 8 carbons, such as a methoxy group, an ethoxy group, a propoxy group, an isopropoxy group, a butoxy group, an isobutoxy group, a sec-butoxy group, a tert-butoxy group, a pentyloxy group, an isopentyloxy group, a hexyloxy group, an allyloxy, group, a 2-propynyloxy group, a benzyloxy group, a phenoxy group or the like.

A “C3-8 cycloalkylidene group” according to the invention is a divalent group formed from cycloalkanes of 3 to 8 carbons by removal of two hydrogen atoms from the same carbon atom, the free valencies of which are part of a double bond. Examples of C3-8 cycloalkylidene groups are such as a cyclopentylidene group, a cyclohexylidene group, a cycloheptylidene group or the like.

A “protecting group for a hydroxyl group” according to the invention is a substituent which is useful for protecting a hydroxyl group, such as a methoxymethyl group, a methylthiomethyl group, a (phenyldimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group, a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-triflluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyldimethylsilyl group, a t-buthyidiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group or the like.

The term “pharmaceutically acceptable prodrug” or “prodrug,” as used herein, represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to a parent compound of formula (I), for example, by hydrolysis in blood. A through discussion is provided in T. Higuchi and V. Stella, Pro-drugs as Novel Delivery Systems, V. 14 of the A.C.S. Symposium Series, and in Edward B. Roche, ed., Bioreversible Carriers in drug Design, American Pharmaceutical Association and Pergamon Press (1987), hereby incorporated by reference.

The present invention offers bisphenyl compounds of formula I.

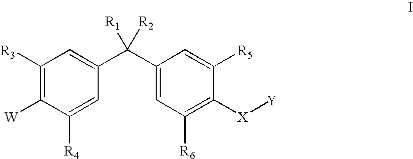

In the above fomula I, X is an optionally substituted methylene, an optionally substituted ethylene, an optionally substituted vinylene, an ethynylene, —S(O)n—, —NH—, or —O—. Preferable X is an optionally substituted methylene, an optionally substituted ethylene, an optionally substituted vinylene or an ethynylene. More preferable X is a methylene, an ethylene, a vinylene or an ethynylene a methylene. Furthermore preferable X is an ethylene, a vinylene or an ethynylene a methylene.

“An optionally substituted methylene” is, in particular, a methylene which may be substituted with a halogen atom(s) and/or a C1-4 alkyl group(s), and is preferablely a methylene.

“An optionally substituted vinylene” is, in particular, a vinylene which may be substituted with a halogen atom(s) and/or a C1-4 alkyl group(s), and is preferablely a vinylene.

“An optionally substituted ethylene” is, in particular, an ethylene which may be substituted with a halogen atom(s) and/or a C1-54 alkyl group(s), and is preferablely an ethylene.

In “—S(O)n-”, n is an integer of 0 to 2, therefore, “—S(O)n-” means —S—, —SO— or —SO₂—.

In the above fomula I, Y is COOR8, CON(R9)R10, S(O)mR11 or a substituent represented by following formula: 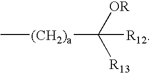
Preferable Y is a substituent represented by following formula:

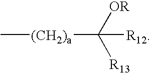

In “COOR8”, R8 is selected an optionally substituted C1-10 alkyl group or an optionally substituted C3-10 cycloalkyl group. In this case, “an optionally substituted C1-10 alkyl group” is, in particular, a C1-10 alkyl group which may be substituted with a halogen atom(s), a hydroxyl group(s), an amino group(s), a carboxyl group(s) and/or a C1-8 alkoxy group(s), and is preferablely a C1-10 alkyl group. “An optionally substituted C3-10 cycloalkyl group” is, in particular, a C3-10 cycloalkyl group which may be substituted with a halogen atom(s), a hydroxyl group(s), an amino group(s), a carboxyl group(s) and/or a C1-8 alkoxy group(s), and is preferablely a C3-10 cycloalkyl group.

In “CON(R9)R10”, R9 and R10 are each independently selected from a hydrogen atom, an optionally substituted C1-10 alkyl group or an optionally substituted C3-10 cycloalkyl group. In this case, “an optionally substituted C1-10 alkyl group” is, in particular, a C1-10 alkyl group which may be substituted with a halogen atom(s), a hydroxyl group(s), an amino group(s), a carboxyl group(s) and/or a C1-8 alkoxy group(s), and is preferablely a C1-10 alkyl group. “An optionally substituted C3-10 cycloalkyl group” is, in particular, a C3-10 cycloalkyl group which may be substituted with a halogen atom(s), a hydroxyl group(s), an amino group(s), a carboxyl group(s) and/or a C1-8 alkoxy group(s), and is preferablely a C3-10 cycloalkyl group.

In “S(O)mR11”, m is an integer of 0 to 2, therefore, “S(O)mR11” means —S—R11, —SO—R11 or —SO₂—R11, and R11 is selected an optionally substituted C1-10 alkyl group or an optionally substituted C3-10 cycloalkyl group. In this case, “an optionally substituted C1-10 alkyl group” is, in particular, a C1-10 alkyl group which may be substituted with a halogen atom(s), a hydroxyl group(s), an amino group(s), a carboxyl group(s) and/or a C1-8 alkoxy group(s), and is preferablely a C1-10 alkyl group. “An optionally substituted C3-10 cycloalkyl group” is, in particular, a C3-10 cycloalkyl group which may be substituted with a halogen atom(s), a hydroxyl group(s), an amino group(s), a carboxyl group(s) and/or a C1-8 alkoxy group(s), and is preferablely a C3-10 cycloalkyl group.

When Y is a substituent represented by following formula: 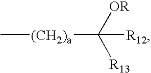
a is an integer of 0 to 3, and is preferablely 0.

R is a hydrogen atom or a protecting group for a hydroxyl group. As a modulator of the vitamin D receptor activity, R is preferablely a hydrogen atom. The compound wherein R is a protecting group for a hydroxyl group is useful as a synthetic intermediate of a modulator of the vitamin D receptor activity. However, some of these compounds exhibit the vitamin D receptor modulating activity, and such compounds are also useful as a modulator of the vitamin D receptor activity.

“A protecting group for a hydroxyl group” is, in particular, a methoxymethyl group, a methylthiomethyl group, a (phenyldimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group- a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-triflluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyidimethylsilyl group, a t-buthyidiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group or the like. “A protecting group for a hydroxyl group” is preferablely selected from a methoxymethyl group, a 2-(trimethylsilyl)ethoxymethyl group, a tetrahydropyranyl group, a benzyl group, a p-methoxybenzyl group, a trimethylsilyl group, a triethylsilyl group, a t-butyldimethylsilyl group, a t-buthyidiphenylsilyl group, an acetyl group, a pivaloyl group, a benzoyl group, a methanesulfonyl group, a tosyl group or a trifluoromethanesulfonyl group.

R12 and R13 are each independently selected from the group consisting of a hydrogen atom, an optionally substituted C1-10 alkyl group, an optionally substituted C3-10 cycloalkyl group, an optionally substituted C2-10 alkenyl group, an optionally substituted C2-10 alkynyl group, or R12 and R13 may together form an optionally substituted C3-C12 cycloalkyl group or an optionally substituted 3-12 membered heterocyclic group. R12 and R13 are preferablely each independently selected from the group consisting of a hydrogen atom, an optionally substituted C1-10 alkyl group, an optionally substituted C3-10 cycloalkyl group, an optionally substituted C2-10 alkenyl group, an optionally substituted C2-10 alkynyl group, or R12 and R13 may together form an optionally substituted C3-C12 cycloalkyl group. R12 and R13 are more preferablely each independently selected from the group consisting of a hydrogen atom, a C1-8 alkyl group which may be substituted with a halogen atom(s), a C3-8 cycloalkyl group which may be substituted with a C1-4 alkyl group, or R12 and R13 are together form a C3-8 cycloalkyl group which may be substituted with a C1-4 alkyl group. R12 and R13 are furthermore preferablely each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group. Especially, R12 and R13 are preferablely selected from the group consisting of i) one of R12 and R13 is a hydrogen atom and the other is a C1-6 alkyl group, ii) one of R12 and R13 is a hydrogen atom and the other is a C3-8 cycloalkyl group which may be substituted with a C1-4 alkyl group, iii) both of R12 and R13 are same and a C1-6 alkyl group which may be substituted with a halogen atom(s), or R12 and R13 are together form a C3-10 cycloalkyl group. One of the preferred combinations of R12 and R13 are, in particular, a hydrogen atom and a tert-butyl group, or both a trifluoromethyl group.

In this case, “an optionally substituted C1-10 alkyl group” is, in particular, a C1-10 alkyl group which may be substituted with a halogen atom(s), a hydroxyl group(s), an amino group(s), a carboxyl group(s) and/or a C1-8 alkoxy group(s), and is preferablely a C1-10 alkyl group. “An optionally substituted C1-10 alkenyl group” is, in particular, a C1-10 alkenyl group which may be substituted with a halogen atom(s), a hydroxyl group(s), an amino group(s), a carboxyl group(s) and/or a C1-8 alkoxy group(s), and is preferablely a C1-10 alkenyl group. “An optionally substituted C1-10 alkynyl group” is, in particular, a C1-10 alkynyl group which may be substituted with a halogen atom(s), a hydroxyl group(s), an amino group(s), a carboxyl group(s) and/or a C1-8 alkoxy group(s), and is preferablely a C1-10 alkynyl group. “An optionally substituted C3-10 cycloalkyl group” is, in particular, a C3-10 cycloalkyl group which may be substituted with a halogen atom(s), a hydroxyl group(s), an amino group(s), a carboxyl group(s), a C1-4 alkyl group and/or a C1-8 alkoxy group(s), and is preferablely a C3-10 cycloalkyl group which may be substituted with a C1-4 alkyl group. “An optionally substituted 3-12 membered heterocyclic group” is, in particular, a 3-12 membered heterocyclic group which may be substituted with a halogen atom(s), a hydroxyl group(s), an amino group(s), a carboxyl group(s) and/or a C1-8 alkoxy group(s).

In the above fomula I, W is a hydroxyl group, a carboxyl group, a trifluoromethanesulfonyloxy group or a substituent represented by following formula: 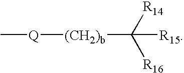
As a modulator of the vitamin D receptor activity, W is preferablely a substituent represented by following formula: 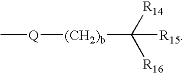
Among them, especially as pharmaceuticals for the treatment of osteoporosis, the compound having a carboxyl group at its terminal end is preffered. On the other hand, the compound wherein W is a hydroxyl group, a carboxyl group or a trifluoromethanesulfonyloxy group is useful as a synthetic intermediate of a modulator of the vitamin D receptor activity. However, some of these compounds exhibit the vitamin D receptor modulating activity, and such compounds are also useful as a modulator of the vitamin D receptor activity.

When Y is a substituent represented by following formula: 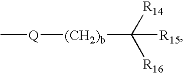
b is an integer of 0 to 10, is preferablely an integer of 0 to 5, and is more preferablely 0, 1 or 2.

Q is —O—, —S—, —NH—, an optionally substituted methylene, an optionally substituted ethylene, an optionally substituted vinylene, an ethynylene, —(CH2)ₖ-NHC(═O)—, —(CH2)ₖ-C(═O)NH—, —(CH2)ₖ-NHC(═O)NH—, —O—(CH2)ₖ-NHC(═O)—, —O—(CH2)ₖ-C(═O)NH—, —O—(CH2)ₖ-NHC(═O)NH— or —(CH2)ₖ-SO₂—. Preferable Q is selected from —O—, a methylene, an ethylene, a vinylene, an ethynylene, —(CH2)ₖ-C(═O)NH— or —O—(CH2)ₖ-C(═O)NH—. In this case, k is an integer of 0 to 2, and is preferablely 1.

“An optionally substituted methylene” is, in particular, a methylene which may be substituted with a halogen atom(s) and/or a C1-4 alkyl group(s), and is preferablely a methylene.

“An optionally substituted vinylene” is, in particular, a vinylene which may be substituted with a halogen atom(s) and/or a C1-4 alkyl group(s), and is preferablely a vinylene.

“An optionally substituted ethylene” is, in particular, an ethylene which may be substituted with a halogen atom(s) and/or a C1-4 alkyl group(s), and is preferablely an ethylene.

R14 is a hydrogen atom, a hydroxyl group, an optionally substituted carboxyl group, an optionally substituted carbamoyl group, an optionally substituted C1-6 alkyl group, an optionally substituted C3-C12 cycloalkyl group, an optionally substituted C1-6 alkenyl group, an optionally substituted C1-6 alkynyl group, an optionally substituted C6-C12 aryl group, an optionally substituted (C6-C12)aryl-(C1-4)alkyl group, —OR17 or —N(R18)R19. R14 is preferablely a hydrogen atom, a hydroxyl group, a carboxyl group which may be substituted with a C1-4 alkyl group, a carbamoyl group which may be substituted with a C1-4 alkyl group, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, —OR17 or —N(R18)R19. R14 is more preferablely a hydrogen atom, a hydroxyl group, a carboxyl group, a carbamoyl group, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, —OR17 or —N(R18)R19.

In “—OR17”, R17 is an optionally substituted C1-6 alkyl group or an optionally substituted C3-C6 cycloalkyl group. R17 is preferablely a C1-4 alkyl group. In this case, “an optionally substituted C1-6 alkyl group” is, in particular, a C1-6 alkyl group which may be substituted with a halogen atom(s), a hydroxyl group(s), an amino group(s), a carboxyl group(s) and/or a C1-8 alkoxy group(s), and is preferablely a C1-6 alkyl group. “An optionally substituted C3-6 cycloalkyl group” is, in particular, a C3-6 cycloalkyl group which may be substituted with a halogen atom(s), a hydroxyl group(s), an amino group(s), a carboxyl group(s) and/or a C1-8 alkoxy group(s), and is preferablely a C3-6 cycloalkyl group.

In “—N(R18)R19”, R18 and R19 are each independently selected from the group consisting of a hydrogen atom, an optionally substituted C1-6 alkyl group, an optionally substituted C3-C6 cycloalkyl group, or R18 and R19 may together form an optionally substituted C3-C12 cycloalkyl group or an optionally substituted 3-12 membered heterocyclic group. R18 and R19 are preferablely each independently selected from a hydrogen atom, an optionally substituted C1-6 alkyl group, an optionally substituted C3-C6 cycloalkyl group. R18 and R19 are more preferablely each independently selected from a hydrogen atom or a C1-4 alkyl group. Especially, it is preferred that R18 is a hydrogen atom and R19is a hydrogen atom, i.e. “—N(R18)R19” represents an amino group. In this case, “an optionally substituted C1-6 alkyl group” is, in particular, a C1-6 alkyl group which may be substituted with a halogen atom(s), a hydroxyl group(s), an amino group(s), a carboxyl group(s) and/or a C1-8 alkoxy group(s), and is preferablely a C1-6 alkyl group. “An optionally substituted C3-6 cycloalkyl group” is, in particular, a C3-6 cycloalkyl group which may be substituted with a halogen atom(s), a hydroxyl group(s), an amino group(s), a carboxyl group(s) and/or a C1-8 alkoxy group(s), and is preferablely a C3-6 cycloalkyl group. “An optionally substituted C3-12 cycloalkyl group” is, in particular, a C3-12 cycloalkyl group which may be substituted with a halogen atom(s), a hydroxyl group(s), an amino group(s), a carboxyl group(s) and/or a C1-8 alkoxy group(s), and is preferablely a C3-12 cycloalkyl group.

R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group, or R15 and R16 may together form ═O. R15 and R16 are preferablely each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group selected from the group consisting of an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group, an isoquinoline group, a cinnoline group, a phthalazine group, a quinazoline group, a quinoxaline group, or R15 and R16 may together form ═O. R15 and R16 are more preferablely each independently selected from the group consisting of a hydrogen atom, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, a 3-8 membered heterocyclic group which may be substituted with a C1-4 alkyl group, a hydroxyl group, a carboxyl group, a carbamoyl group or an amino group, wherein said heterocyclic group selected from the group consisting of an oxetarie group, a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a pyridine group, a pyridazine group, a pyrimidine group, a pyrazine group, a tetrahydropyran group, a pyran group, a thiopyran group, or R15 and R16 may together form ═O. R15 and R16 are furthermore preferablely each independently selected from the group consisting of a hydrogen atom, a C1-6. alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, a 3-8 membered heterocyclic group selected from the group consisting of a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a pyridine group, a tetrahydropyran group.

Regarding R14, R15 and R16, it is preferred that at least one of R14, R15 or R16 is a hydrogen atom, and/or at least one of R14, R15 or R16 is a substituent which have a hydroxyl group, a carboxyl group, a carbamoyl group or an amino group. Also, it is preferred that R14 is a hydroxyl group, and/or one of R15 and R16 is a hydrogen atom and the other is a C1-6 alkyl group substituted with a carboxyl group.

Or one of (R15 and R17), (R16 and R17), (R15 and R18), (R16 and R18), (R15 and R19) or (R16 and R19) may together form a 3-12 membered cyclic ring which is selected from the group consisting of an amidine ring, an amine ring, an ether ring, a lactam ring, a lactone ring, an acetal ring, a hemiacetal ring, a carbonate ring, a carbamate ring, an urea ring, combinations thereof. In this case, said cyclic ring is preferablely a lactam ring or a lactone ring, and is more preferablely a lactone ring.

In the above fomula I, R1 and R2 are each independently selected from the group consisting of a C1-6 alkyl group optionally substituted with a halogen atom(s), a C3-6 cycloalkyl group optionally substituted with a halogen atom(s), a C2-6 alkenyl group optionally substituted with a halogen atom(s), a C2-6 alkynyl group optionally substituted with a halogen atom(s), or R1 and R2 may together form a C3-8 cycloalkyl group optionally substituted with a halogen atom(s), a C3-8 cycloalkenyl group optionally substituted with a halogen atom(s) or a C3-8 cycloalkylidene group optionally substituted with a halogen atom(s).

Preferable R1 is a C1-6 alkyl group, more preferable R1 is a C1-4 alkyl group, and the most preferable R1 is an ethyl group.

Preferable R2 is a C1-6 alkyl group, more preferable R2 is a C1-4 alkyl group, and the most preferable R2 is an ethyl group.

In the above fomula I, R3, R4, R5 and R6 are each independently selected from a hydrogen atom, a halogen atom, a C1-6 alkyl group optionally substituted with a halogen atom(s) or a C3-6 cycloalkyl group optionally substituted with a halogen atom(s).

Preferable R3 is a hydrogen atom or a C1-6 alkyl group, more preferable R3 is a hydrogen atom or a C1-4 alkyl group, and the most preferable R3 is a hydrogen atom or a methyl group.

Preferable R4 is a halogen atom or a C1-6 alkyl group, more preferable R4 is a halogen atom or a C1-4 alkyl group, and the most preferable R4 is a chlorine atom or a methyl group.

Preferable R5 is a hydrogen atom.

Preferable R6 is a halogen atom or a C1-6 alkyl group, more preferable R6 is a halogen atom or a C1-4 alkyl group, and the most preferable R6 is a chlorine atom, a methyl group, an ethyl group, a propyl group or an isopropyl group.

When X is —S(O)n-, —NH—or —O—, Q is selected from a methylene which may be substituted an C1-4 alkyl group, an ethylene, a vinylene, an ethynylene, —(CH²)ₖ-NHC(═O)—, —(CH2)ₖ-C(═O)NH—, —(CH2)ₖ-NHC(═O)NH— or —(CH2)ₖ-SO₂—;

When Q is —O—, —S—, —NH—, —O—(CH2)ₖ-NHC(═O)—, —O—(CH²)ₖ-C(═O)NH— or —O—(CH2)ₖ-NHC(═O)NH—, X is selected from an optionally substituted methylene, an optionally substituted ethylene, an optionally substituted vinylene or an ethynylene.

Examples of the preferred compounds of the present invention are represented as follows.

(1) The compound of the above fomula I wherein

X is an optionally substituted methylene, an optionally substituted ethylene, an optionally substituted vinylene or an ethynylene;

Y is COOR8, CON(R9)R10, S(O)mR11 or a substituent represented by following formula:

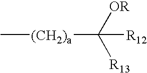

R is a hydrogen atom;

R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;

W is a substituent represented by following formula:

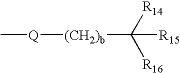

Q is —O—, a methylene, an ethylene, a vinylene, an ethynylene, —(CH2)ₖ-C(═O)NH— or —O—(CH2)ₖ-C(═O)NH—;

R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group selected from the group consisting of an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group, an isoquinoline group, a cinnoline group, a phthalazine group, a quinazoline group, a quinoxaline group, or R15 and R16 may together form ═O;

R17 is an optionally substituted C1-6 alkyl group or an optionally substituted C3-C6 cycloalkyl group;

R18 and R19 are each independently selected from a hydrogen atom, an optionally substituted C1-6 alkyl group or an optionally substituted C3-C6 cycloalkyl group;

or one of (R15 and R17), (R16 and R17), (R15 and R18), (R16 and R18), (R15 and R19) or (R16 and R19) may together form a 3-12 membered cyclic ring which is selected from a lactam ring or a lactone ring;

R1 is a C1-6 alkyl group;

R2 is a C1-6 alkyl group;

R3 is a hydrogen atom or a C1-6 alkyl group;

R4 is a halogen atom or a C1-6 alkyl group;

R5 is a hydrogen atom;

R6 is a halogen atom or a C1-6 alkyl group.

(2) The compound according to (1) wherein

X is an ethylene, a vinylene, or an ethynylene;

Y is a substituent represented by following formula:

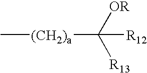

R is a hydrogen atom;

R12 and R13 are each independently selected from the group consisting of a hydrogen atom, a C1-8 alkyl group which may be substituted with a halogen atom(s), a C3-8 cycloalkyl group which may be substituted with a C1-4 alkyl group, or R12 and R13 are together form a C3-8 cycloalkyl group which may be substituted with a C1-4 alkyl group;

W is a substituent represented by following formula:

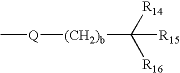

Q is —O—, a methylene, an ethylene, a vinylene, an ethynylene, —(CH2)ₖ-C(═O)NH— or —O—(CH2)ₖ-C(═O)NH—;

b is an integer of 0 to 5;

R14 is a hydrogen atom, a hydroxyl group, a carboxyl group which may be substituted with a C1-4 alkyl group, a carbamoyl group which may be substituted with a C1-4 alkyl group, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, —OR17 or —N(R18)R19;

R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, a 3-8 membered heterocyclic group which may be substituted with a C1-4 alkyl group, a hydroxyl group, a carboxyl group, a carbamoyl group or an amino group, wherein said heterocyclic group selected from the group consisting of an oxetane group, a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a pyridine group, a pyridazine group, a pyrimidine group, a pyrazine group, a tetrahydropyran group, a pyran group, a thiopyran group, or R15 and R16 may together form ═O;

and at least one of R14, R15 or R16 is a hydrogen atom;

R17 is a C1-4 alkyl group;

R18 and R19 are each independently selected from a hydrogen atom or a C1-4 alkyl group;

or one of (R15 and R17), (R16 and R17), (R15 and R18), (R16 and R18), (R15 and R19) or (R16 and R19) may together form a 3-12 membered lactone ring.

(3) The compound according to (2) wherein

R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;

W is a substituent represented by following formula:

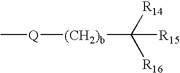

Q is —O—, —(CH2)ₖ-C(═O)NH— or —O—(CH2)ₖ-C(═O)NH—;

b is 0, 1 or 2;

k is 1;

R14 is a hydrogen atom, a hydroxyl group, a carboxyl group, a carbamoyl group, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, —OR17 or —N(R18)R19;

R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, a 3-8 membered heterocyclic group selected from the group consisting of a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a pyridine group, a tetrahydropyran group;

and at least one of R14, R15 or R16 is a hydrogen atom;

R18 is a hydrogen atom;

R19 is a hydrogen atom;

or one of (R15 and R17), (R16 and R17), (R15 and R18), (R16 and R18), (R15 and R19) or (R16 and R19) may together form a 3-12 membered lactone ring;

R1 is a C1-4 alkyl group;

R2 is a C1-4 alkyl group;

R3 is a hydrogen atom or a C1-4 alkyl group;

R4 is a halogen atom or a C1-4 alkyl group;

R5 is a hydrogen atom;

R6 is a halogen atom or a C1-4 alkyl group.

(4) The compound according to (3) wherein

R12 and R13 are selected from the group consisting of one of R12 and R13 is a hydrogen atom and the other is a C1-6 alkyl group,

one of R12 and R13 is a hydrogen atom and the other is a C3-8 cycloalkyl group which may be substituted with a C1-4 alkyl group, both of R12 and R13 are same and a C1-6 alkyl group which may be substituted with a halogen atom(s),

or R12 and R13 are together form a C3-10 cycloalkyl group;

Q is —O— or —O—(CH2)ₖ-C(═O)NH—;

R1 is an ethyl group;

R2 is an ethyl group;

R3 is a hydrogen atom or a methyl group;

R4 is a chlorine atom or a methyl group;

R6 is a chlorine atom, a methyl group, an ethyl group, a propyl group or an isopropyl group.

(5) The compound according to (4) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol, (R)-5-(2-Chloro-4-{1-[3-chloro-4-((R)-3-hydroxy-4,4-dimethyl-pentyl)-phenyl]-1-ethyl-propyl}-phenoxymethyl)-dihydro-furan-2-one, (R)-5-(2-Chloro-4-{1-[3-chloro-4-((S)-3-hydroxy-4,4-dimethyl-pentyl)-phenyl]-1-ethyl-propyl}-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (R)-5-(4-{1-Ethyl-1-[4-((E)-3-ethyl-3-hydroxy-pent-l -enyl)-3-methyl-phenyl]-propyl}-2-methyl-phenoxymethyl)-dihydro-furan-2-one, (R)-5-(4-{1-Ethyl-1-[4-(3-ethyl-3-hydroxy-pent-1-ynyl)-3-methyl-phenyl]-propyl}-2-methyl-phenoxymethyl)-dihydro-furan-2-one, (R)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (R)-5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclohexyl)-propyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopentyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopentyl)-propyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-propyl]-3-methyl-phenyl}-propyl)-2-methyl-phenoxymethyl]-dihydro-furan-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (R)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one, (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one, (S)-5-(4-{1-Ethyl-1-[3-methyl-4-(2-methyl-propane-2-sulfinylmethyl)-phenyl]-propyl}-2-methyl-phenoxymethyl)-dihydro-furan-2-one, (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one, (S)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one, (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-non-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-en-4-ynyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-methyl-but-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-propyl-hex-1-enyl)-3-methyl-phenyl]-propyl}-2-methyl-phenoxymethyl)-dihydro-furan-2-one, (S)-5-(4-{1-Ethyl-1-[4-((E)-3-hydroxy-4,4-dimethyl-pent-1-enyl)-3-methyl-phenyl]-propyl}-2-methyl-phenoxymethyl)-dihydro-furan-2-one, (S)-5-(4-{1-Ethyl-1-[4-(1-hydroxy-cyclobutylethynyl)-3-methyl-phenyl]-propyl}-2-methyl-phenoxymethyl)-dihydro-furan-2-one, (S)-5-(4-{1-Ethyl-1-[4-(1-hydroxy-cyclohexylethynyl)-3-methyl-phenyl]-propyl}-2-methyl-phenoxymethyl)-dihydro-furan-2-one, (S)-5-(4-{1-Ethyl-1-[4-(1-hydroxy-cyclopentylethynyl)-3-methyl-phenyl]-propyl}-2-methyl-phenoxymethyl)-dihydro-furan-2-one, (S)-5-(4-{1-Ethyl-1-[4-(3-ethyl-3-hydroxy-pentyl)-3-methyl-phenyl]-propyl}-2-methyl-phenoxymethyl)-dihydro-furan-2-one, (S)-5-(4-{1-Ethyl-1-[4-(3-hydroxy-3-methyl-but-1-ynyl)-3-methyl-phenyl]-propyl}-2-methyl-phenoxymethyl)-dihydro-furan-2-one, (S)-5-(4-{1-Ethyl-1-[4-(3-hydroxy-3-propyl-hex-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-decyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-heptyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-nonyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-octyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pent-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (S)-5-[4-(1-Ethyl-1-{4-[(E)-2-(1-hydroxy-cyclobutyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclohexyl)-vinyl]-3-methyl-phenyl)-propyl)-2-methyl-phenoxymethyl]-dihydro-furan-2-one, (S)-5-[4-(1-Ethyl-1-{4-[(E)-2-(1-hydroxy-cyclopentyl)-vinyl]-3-methyl-phenyl}-propyl)-2-methyl-phenoxymethyl]-dihydro-furan-2-one, (S)-5-{4-[1-(4-tert-Butylsulfanylmethyl-3-methyl-phenyl)-1-ethyl-propyl]-2-methyl-phenoxymethyl}-dihydro-furan-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[4-((R)-3-hydroxy-4,4-dimethyl-pentyl)-3-methyl-phenyl]-propyl}-2-methyl-phenoxymethyl)-tetrahydro-pyran-2-one, (S)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one.

(6) The compound according to (4) wherein R3 is a hydrogen atom.

(7) The compound according to (6) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-(1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(8) The compound according to (4) wherein R4 is a chlorine atom.

(9) The compound according to (8) selected from 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-l -ethyl-propyl}-phenoxy)-4(R)-hydroxy-pentanoic acid sodium salt.

(10) The compound according to (4) wherein R6 is a chlorine atom.

(11) The compound according to (10) selected from the group consisting of 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt.

(12) The compound according to (4) wherein R6 is a methyl group.

(13) The compound according to (12) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl- but-1-ynyl)- phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2- (R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2- (S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid.

(14) The compound according to (4) wherein X is an ethylene.

(15) The compound according to (14) selected from the group consisting of (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 1-(4-{1-[4-(5-Amino-pentyloxy)-3-methyl-phenyl]-1-ethyl-propyl}-2-methyl-phenyl)-4,4-dimethyl-pentan-3-ol, 2- (R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(16) The compound according to (4) wherein X is a vinylene.

(17) The compound according to (16) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid.

(18) The compound according to (4) wherein X is an ethynylene.

(19) The compound according to (18) selected from the group consisting of (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol.

(20) The compound according to (4) wherein one of R12 and R13 is a hydrogen atom and the other is a C1-6 alkyl group.

(21) The compound according to (20) wherein one of R12 and R13 is a hydrogen atom and the other is a tert-butyl group.

(22) The compound according to (21) selected from the group consisting of (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 1-(4-{1-[4-(5-Amino-pentyloxy)-3-methyl-phenyl]-1-ethyl-propyl}-2-methyl-phenyl)-4,4-dimethyl-pentan-3-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 2- (R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2- (S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid.

(23) The compound according to (4) wherein both of R12 and R13 are same and a C1-6 alkyl group which may be substituted with a halogen atom(s).

(24) The compound according to (23) wherein R12 is a trifluoromethyl group and R13 are is a trifluoromethyl group.

(25) The compound according to (24) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(26) The compound according to (4) wherein Q is —O—.

(27) The compound according to (26) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-l -ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(28) The compound according to (4) wherein at least one of R14, R15 or R16 is a substituent which have a hydroxyl group, a carboxyl group, a carbamoyl group or an amino group.

(29) The compound according to (28) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[⁴-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2- (R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2- (S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(30) The compound according to (4) wherein R14 is a hydroxyl group.

(31) The compound according to (30) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl)-propyl}-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

(32) The compound according to (4) wherein one of R15 and R16 is a hydrogen atom and the other is a C1-6 alkyl group substituted with a carboxyl group.

(33) The compound according to (32) selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid.

(34) The compound according to (4) wherein one of (R15 and R17), (R16 and R17), (R15 and R18), (R16 and R18), (R15 and R19) or (R16 and R19) together form a 3-12 membered lactone ring.

(35) The compound according to (34) selected from the group consisting of (R)-5-(2-Chloro-4-{1-[3-chloro-4-((R)-3-hydroxy-4,4-dimethyl-pentyl)-phenyl]-1-ethyl-propyl}-phenoxymethyl)-dihydro-furan-2-one, (R)-5-(2-Chloro-4-{1-[3-chloro-4-((S)-3-hydroxy-4,4-dimethyl-pentyl)-phenyl]-1-ethyl-propyl}-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[4-(3-ethyl-3-hydroxy-pent-1-ynyl)-3-methyl-phenyl]-propyl}-2-methyl-phenoxymethyl)-dihydro-furan-2-one, (R)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (R)-5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclohexyl)-propyl]-3-methyl-phenyl)-propyl}-2-methyl-phenoxymethyl]-dihydro-furan-2-one, (R)-5-[4-(1-Ethyl-1-{4-[3-hydroxy-3-(1-methyl-cyclopentyl)-prop-1-ynyl]-3-methyl-phenyl}-propyl)-2-methyl-phenoxymethyl]-dihydro-furan-2-one, (R)-5-[4-(l -Ethyl-1-{4-[3-hydroxy-3-(1-methyl-cyclopentyl)-propyl]-3-methyl-phenyl}-propyl)-2-methyl-phenoxymethyl]-dihydro-furan-2-one, (R)-5-[4-1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-propyl]-3-methyl-phenyl}-propyl)-2-methyl-phenoxymethyl]-dihydro-furan-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (R)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one, (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one, (S)-5-(4-{1-Ethyl-1-[3-methyl-4-(2-methyl-propane-2-sulfinylmethyl)-phenyl]-propyl}-2-methyl-phenoxymethyl)-dihydro-furan-2-one, (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one, (S)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one, (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-non-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-en-4-ynyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-methyl-but-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-propyl-hex-1-enyl)-3-methyl-phenyl]-propyl}-2-methyl-phenoxymethyl)-dihydro-furan-2-one, (S)-5-(4-{1-Ethyl-1-[4-((E)-3-hydroxy-4,4-dimethyl-pent-1-enyl)-3-methyl-phenyl]-propyl}-2-methyl-phenoxymethyl)-dihydro-furan-2-one, (S)-5-(4-{1-Ethyl-1-[4-(1-hydroxy-cyclobutylethynyl)-3-methyl-phenyl]-propyl}-2-methyl-phenoxymethyl)-dihydro-furan-2-one, (S)-5-(4-{1-Ethyl-1-[4-(1-hydroxy-cyclohexylethynyl)-3-methyl-phenyl]-propyl}-2-methyl-phenoxymethyl)-dihydro-furan-2-one, (S)-5-(4-{1-Ethyl-1-[4-(1-hydroxy-cyclopentylethynyl)-3-methyl-phenyl]-propyl}-2-methyl-phenoxymethyl)-dihydro-furan-2-one, (S)-5-(4-{1-Ethyl-1-[4-(3-ethyl-3-hydroxy-pentyl)-3-methyl-phenyl]-propyl}-2-methyl-phenoxymethyl)-dihydro-furan-2-one, (S)-5-(4-{1-Ethyl-1-[4-(3-hydroxy-3-methyl-but-1-ynyl)-3-methyl-phenyl]-propyl}-2-methyl-phenoxymethyl)-dihydro-furan-2-one, (S)-5-(4-{1-Ethyl-1-[4-(3-hydroxy-3-propyl-hex-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-decyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-heptyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-nonyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-octyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pent-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (S)-5-[4-(1-Ethyl-1-{4-[(E)-2-(1-hydroxy-cyclobutyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclohexyl)-vinyl]-3-methyl-phenyl)-propyl)-2-methyl-phenoxymethyl]-dihydro-furan-2-one, (S)-5-[4-(1-Ethyl-1-{4-[(E)-2-(1-hydroxy-cyclopentyl)-vinyl]-3-methyl-phenyl}-propyl)-2-methyl-phenoxymethyl]-dihydro-furan-2-one, (S)-5-{4-[1-(4-tert-Butylsulfanylmethyl-3-methyl-phenyl)-1-ethyl-propyl]-2-methyl-phenoxymethyl}-dihydro-furan-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[4-((R)-3-hydroxy-4,4-dimethyl-pentyl)-3-methyl-phenyl]-propyl}-2-methyl-phenoxymethyl)-tetrahydro-pyran-2-one, (S)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one.

(36) The compound according to (3) wherein

R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),

or R12 and R13 are together form a C3-10 cycloalkyl group;

Q is a methylene, an ethylene, an ethynylene or —(CH2)ₖ-C(═O)NH—;

R1 is an ethyl group;

R2 is an ethyl group;

R3 is a hydrogen atom;

R4 is a chlorine atom or a methyl group;

R6 is a chlorine atom or a methyl group.

(37) The compound according to (36) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

(38) The compound according to (36) wherein R3 is a hydrogen atom. (39) The compound according to (38) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

(40) The compound according to (36) wherein R4 is a chlorine atom.

(41) The compound according to (36) wherein R6 is a chlorine atom.

(42) The compound according to (36) wherein R6 is a methyl group.

(43) The compound according to (42) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

(44) The compound according to (36) wherein X is an ethylene.

(45) The compound according to (44) which is 6-(4-{1-Ethyl-1-[4-(3-hydroxy-4,4-dimethyl-pentyl)-3-methyl-phenyl]-propyl}-2-methyl-phenyl)-hex-5-ynoic acid.

(46) The compound according to (36) wherein X is a vinylene.

(47) The compound according to (46) selected from the group consisting of 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

(48) The compound according to (36) wherein X is an ethynylene.

(49) The compound according to (48) selected from 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid or 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid.

(50) The compound according to (36) wherein one of R12 and R13 is a hydrogen atom and the other is a C1-6 alkyl group.

(51) The compound according to (50) wherein one of R12 and R13 is a hydrogen atom and the other is a tert-butyl group.

(52) The compound according to (51) which is 6-(4-{1-Ethyl-1-[4-(3-hydroxy-4,4,4-dimethyl-pentyl)-3-methyl-phenyl]-propyl}-2-methyl-phenyl)-hex-5-ynoic acid.

(53) The compound according to (36) wherein both of R12 and R13 are same and a C1-6 alkyl group which may be substituted with a halogen atom(s).

(54) The compound according to (53) wherein R12 is a trifluoromethyl group and R13 are is a trifluoromethyl group.

(55) The compound according to (54) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

(56) The compound according to (36) wherein Q is an ethylene.

(57) The compound according to (56) selected from 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid or 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid.

(58) The compound according to (36) wherein Q is an ethynylene.

(59) The compound according to (58) selected from the group consisting of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

(60) The compound according to (36) wherein at least one of R14, R15 or R16 is a substituent which have a hydroxyl group, a carboxyl group, a carbamoyl group or an amino group.

(61) The compound according to (60) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

(62) The compound according to (36) wherein R14 is a hydroxyl group.

(63) The compound according to (36) wherein one of R15 and R16 is a hydrogen atom and the other is a C1-6 alkyl group substituted with a carboxyl group.

(64) The compound according to (63) selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

(65) The compound according to (36) wherein one of (R15 and R17), (R16 and R17), (R15 and R18), (R16 and R18), (R15 and R19) or (R16 and R19) together form a 3-12 membered lactone ring.

Another examples of the preferred compounds of the present invention are represented as follows.

(66) The compound of the above fomula I wherein X is an optionally substituted vinylene, or an ethynylene.

(67) The compound of the above fomula I wherein

W is a substituent represented by following formula:

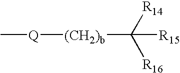

Q is selected from the group consisting of —O—, a methylene, an ethylene, a vinylene, and an ethynylene; and wherein

b is 1;

R14 is a hydroxyl group;

R15 is a hydrogen atom;

R16 is a C1-6 alkyl group substituted with a carboxyl group.

(68) The compound of the above fomula I selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-ethyl-3-hydroxy-pent-1-ynyl)-3-methyl-phenyl]-propyl}-2-methyl-phenoxy)-4-hydroxy-pentanoic acid, (R)-5-(4-{1-Ethyl-1-[4-(3-ethyl-3-hydroxy-pent-1-ynyl)-3-methyl-phenyl]-propyl}-2-methyl-phenoxy)-4-hydroxy-pentanoic acid, (S)-6-(4-{1-Ethyl-1-[4-(3-ethyl-3-hydroxy-pent-1-ynyl)-3-methyl-phenyl]-propyl}-2-methyl-phenoxy)-5-hydroxy-hexanoic acid, (R)-6-(4-{1-Ethyl-1-[4-(3-ethyl-3-hydroxy-pent-1-ynyl)-3-methyl-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, 2-(4-{1-Ethyl-1-[4-(3-ethyl-3-hydroxy-pent-1-ynyl)-3-methyl-phenyl]-propyl}-2-methyl-phenoxymethyl)-propane-1,3-diol, 4-{1-Ethyl-1-[4-(3-ethyl-3-hydroxy-pent-1-ynyl)-3-methyl-phenyl]-propyl}-2-methyl-phenol, 5-(4-{1-Ethyl-1-[4-(3-ethyl-3-hydroxy-pent-1-ynyl)-3-methyl-phenyl]-propyl}-2-methyl- phenoxy)-pentanoic acid, 6-(4-{1-Ethyl-1-[4-(3-ethyl-3-hydroxy-pent-1-ynyl)-3-,methyl-phenyl]-propyl}-2-methyl-phenoxy)-hexanoic acid, 3-(4-{1-Ethyl-1-[4-(3-ethyl-3-hydroxy-pent-1-ynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-3-ethyl-pent-1-yn-3-ol, 3-Ethyl-1-[4-(1-ethyl-1-{3-methyl-4-[4-(1H-tetrazol-5-yl)-butoxy]-phenyl}-propyl)-2-methyl-phenyl]-pent-1-yn-3-ol, (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (R)-5-(4-{1-Ethyl-1-[4-((E)-3-ethyl-3-hydroxy-pent-1.-enyl)-3-methyl-phenyl]-propyl}-2-methyl-phenoxy)-4-hydroxy-pentanoic acid, (S)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid, (R)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 4-{1-Ethyl-1-[4-((E)-3-ethyl-3-hydroxy-pent-1-enyl)-3-methyl-phenyl]-propyl}-2-methyl-phenol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 6-(4-{1-Ethyl-1-[4-((E)-3-ethyl-3-hydroxy-pent-1-enyl)-3-methyl-phenyl]-propyl}-2-methyl-phenoxy)-hexanoic acid, 3-(4-{1-Ethyl-1-[4-((E)-3-ethyl-3-hydroxy-pent-1-enyl)-3-methyl-phenyl]-propyl}-2-methyl-phenyl)-propionic acid, (E)-1-(4-{1-[4-(5-Amino-pentyloxy)-3-methyl-phenyl]-1-ethyl-propyl}-2-methyl-phenyl)-3-ethyl-pent-1-en-3-ol, (E)-3-Ethyl-1-[4-(l -ethyl-1-{3-methyl-4-[4-(1H-tetrazol-5-yl)-butoxy]-phenyl}-propyl)-2-methyl-phenyl]-pent-1-en-3-ol, (S)-5-(4-{1-Ethyl-1-[4-(3-ethyl-3-hydroxy-pentyl)-3-methyl-phenyl]-propyl}-2-methyl-phenoxy)-4-hydroxy-pentanoic acid, (R)-5-(4-{1-Ethyl-1-[4-(3-ethyl-3-hydroxy-pentyl)-3-methyl-phenyl]-propyl}-2-methyl-phenoxy)-4-hydroxy-pentanoic acid, (S)-6-(4-{1-Ethyl-1-[4-(3-ethyl-3-hydroxy-pentyl)-3-methyl-phenyl]-propyl)-2-methyl-phenoxy)-5-hydroxy-hexanoic acid, (R)-6-(4-{1-Ethyl-1-[4-(3-ethyl-3-hydroxy-pentyl)-3-methyl-phenyl]-propyl}-2-methyl-phenoxy)-5-hydroxy-hexanoic acid, (S)-3-(4-{1-Ethyl-1-[4-(3-ethyl-3-hydroxy-pentyl)-3-methyl-phenyl]-propyl}-2-methyl-phenoxy)-propane-1,2-diol, 2-(4-{1-Ethyl-1-[4-(3-ethyl-3-hydroxy-pentyl)-3-methyl-phenyl]-propyl}-2-methyl-phenoxymethyl)-propane-1,3-diol, 4-{1-Ethyl-1-[4-(3-ethyl-3-hydroxy-pentyl)-3-methyl-phenyl]-propyl}-2-methyl-phenol, 5-(4-{1-Ethyl-1-[4-(3-ethyl-3-hydroxy-pentyl)-3-methyl-phenyl]-propyl}-2-methyl-phenoxy)-pentanoic acid, 6-(4-{1-Ethyl-1-[4-(3-ethyl-3-hydroxy-pentyl)-3-methyl-phenyl]-propyl}-2-methyl-phenoxy)-hexanoic acid, 3-(4-{1-Ethyl-1-[4-(3-ethyl-3-hydroxy-pentyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-3-ethyl-pentan-3-ol, 3-Ethyl-1-[4-(1-ethyl-1-{3-methyl-4-[4-(1H-tetrazol-5-yl)-butoxy]-phenyl}-propyl)-2-methyl-phenyl]-pentan-3-ol, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid,. (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 4-{1-Ethyl-1-[3-methyl-4-(4,4,4-trifluoro-3-hydroxy-3-trifluoromethyl-but-1-ynyl)-phenyl]-propyl}-2-methyl-phenol, 5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentanoic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-hexanoic acid, 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 4-(4-{1-[4-(5-Amino-pentyloxy)-3-methyl-phenyl]-1-ethyl-propyl}-2-methyl-phenyl)-1,1,1-trifluoro-2-trifluoromethyl-but-3-yn-2-ol, 4-[4-(1-Ethyl-1-{3-methyl-4-[4-(1H-tetrazol-5-yl)-butoxy]-phenyl}-propyl)-2-methyl-phenyl]-, 1,1,1-trifluoro-2-trifluoromethyl-but-3-yn-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, 2-(4-{1-Ethyl-1-[3-methyl-4-((E)-4,4,4-trifluoro-3-hydroxy-3trifluoromethyl-but-1-enyl)-phenyl]-propyl}-2-methyl-phenoxymethyl)-propane-1,3-diol, 4-{1-Ethyl-1-[3-methyl-4-((E)-4,4,4-trifluoro-3-hydroxy-3-trifluoromethyl-but-1-enyl)-phenyl]-propyl}-2-methyl-phenol, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (R)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-6-(4-{1-Ethyl-1-[3-methyl-4-(4,4,4-trifluoro-3-hydroxy-3-trifluoromethyl-butyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-butyl)-phenyl]-propyl}-2-methyl-phenoxy)-5-hydroxy-hexanoic acid, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 4-{1-Ethyl-1-[3-methyl-4-(4,4,4-trifluoro-3-hydroxy-3-trifluoromethyl-butyl)-phenyl]-propyl}-2-methyl-phenol, 5-(4-{1-Ethyl-1-[3-methyl-4-(4,4,4-trifluoro-3-hydroxy-3-trifluoromethyl-butyl)-phenyl]-propyl}-2-methyl-phenoxy)-pentanoic acid, 6-(4-{1-Ethyl-1-[3-methyl-4-(4,4,4-trifluoro-3-hydroxy-3-trifluoromethyl-butyl)-phenyl]-propyl}-2-methyl-phenoxy)-hexanoic acid, 3-(4-{1-Ethyl-1-[3-methyl-4-(4,4,4-trifluoro-3-hydroxy-3-trifluoromethyl-butyl)-phenyl]-propyl}-2-methyl-phenyl)-propionic acid, 4-(4-{1-[4-(5-Amino-pentyloxy)-3-methyl-phenyl]-1-ethyl-propyl}-2-methyl-phenyl)-1,1,1-trifluoro-2-trifluoromethyl-butan-2-ol, 4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-butan-2-ol, (S)-5-(4-{1-Ethyl-1-[4-(3-hydroxy-3-methyl-but-1-ynyl)-3-methyl-phenyl]-propyl}-2-methyl-phenoxy)-4-hydroxy-pentanoic acid, (R)-5-(4-{1-Ethyl-1-[4-(3-hydroxy-3-methyl-but-1-ynyl)-3-methyl-phenyl]-propyl}-2-methyl-phenoxy)-4-hydroxy-pentanoic acid, (S)-6-(4-{1-Ethyl-1-[4-(3-hydroxy-3-methyl-but-1-ynyl)-3-methyl-phenyl]-propyl}-2-methyl-phenoxy)-5-hydroxy-hexanoic acid, (R)-6-(4-{1-Ethyl-1-[4-(3-hydroxy-3-methyl-but-1-ynyl)-3-methyl-phenyl]-propyl}-2-methyl-phenoxy)-5-hydroxy-hexanoic acid, (S)-3-(4-{1-Ethyl-1-[4-(3-hydroxy-3-methyl-but-1-ynyl)-3-methyl-phenyl]-propyl}-2-methyl-phenoxy)-propane-1,2-diol, 2-(4-{1-Ethyl-1-[4-(3-hydroxy-3-methyl-but-1-ynyl)-3-methyl-phenyl]-propyl}-2-methyl-phenoxymethyl)-propane-1,3-diol, 4-{1-Ethyl-1-[4-(3-hydroxy-3-methyl-but-1-ynyl)-3-methyl-phenyl]-propyl}-2-methyl-phenol, 5-(4-{1-Ethyl-1-[4-(3-hydroxy-3-methyl-but-1-ynyl)-3-methyl-phenyl]-propyl}-2-methyl-phenoxy)-pentanoic acid, 6-(4-{1-Ethyl-1-[4-(3-hydroxy-3-methyl-but-1-ynyl)-3-methyl-phenyl]-propyl}-methyl-phenoxy)-hexanoic acid, 3-(4-{1-Ethyl-1-[4-(3-hydroxy-3-methyl-but-1-ynyl)-3-methyl-phenyl]-propyl}-2-methyl-phenyl)-propionic acid, 4-(4-l -[4-(5-Amino-pentyloxy)-3-methyl-phenyl]-1-ethyl-propyl}-2-methyl-phenyl)-2-methyl-but-3-yn-2-ol, 4-[4-(1-Ethyl-1-{3-methyl-4-[4-(1 H-tetrazol-5-yl)-butoxy]-phenyl}-propyl)-2-methyl-phenyl]-2-methyl-but-3-yn-2-ol, (S)-5-(4-{1-Ethyl-1-[4-((E)-3-hydroxy-3-methyl-but-1-enyl)-3-methyl-phenyl]-propyl}-2-methyl-phenoxy)-4-hydroxy-pentanoic acid, (R)-5-(4-{1-Ethyl-1-[4-((E)-3-hydroxy-3-methyl-but-1-enyl)-3-methyl-phenyl]-propyl}-2-methyl-phenoxy)-4-hydroxy-pentanoic acid, (S)-6-(4-{1-Ethyl-1-[4-((E)-3-hydroxy-3-methyl-but-1-enyl)-3-methyl-phenyl]-propyl}-2-methyl-phenoxy)-5-hydroxy-hexanoic acid, (R)-6-(4-{1-Ethyl-1-[4-((E)-3-hydroxy-3-methyl-but-1-enyl)-3-methyl-phenyl]-propyl}-2-methyl-phenoxy)-5-hydroxy-hexanoic acid, (S)-3-(4-{1-Ethyl-1-[4-((E)-3-hydroxy-3-methyl-but-1-enyl)-3-methyl-phenyl]-propyl}-2-methyl-phenoxy)-propane-1,2-diol, 2-(4-{1-Ethyl-1-[4-((E)-3-hydroxy-3-methyl-but-1-enyl)-3-methyl-phenyl]-propyl}-2-methyl-phenoxymethyl)-propane-1,3-diol, 4-{1-Ethyl-1-[4-((E)-3-hydroxy-3-methyl-but-1-enyl)-3-methyl-phenyl]-propyl}-2-methyl-phenol, 5-(4-{1-Ethyl-1-[4-((E)-3-hydroxy-3-methyl-but-1-enyl)-3-methyl-phenyl]-propyl}-2-methyl-phenoxy)-pentanoic acid, 6-(4-{1-Ethyl-1-[4-((E)-3-hydroxy-3-methyl-but-1-enyl)-3-methyl-phenyl]-propyl}-2-methyl-phenoxy)-hexanoic acid, 3-(4-{1-Ethyl-1-[4-((E)-3-hydroxy-3-methyl-but-1-enyl)-3-methyl-phenyl]-propyl}-2-methyl-phenyl)-propionic acid, (E)-4-(4-{1-[4-(5-Amino-pentyloxy)-3-methyl-phenyl]-1-ethyl-propyl}-2-methyl-phenyl)-2-methyl-but-3-en-2-ol, (E)-4-[4-(1-Ethyl-1-{3-methyl-4-[4-(1H-tetrazol-5-yl)-butoxy]-phenyl}-propyl)-2-methyl-phenyl]-2-methyl-but-3-en-2-ol, (S)-5-(4-{1-Ethyl-1-[4-(3-hydroxy-3-methyl-butyl)-3-methyl-phenyl]-propyl}-2-methyl-phenoxy)-4-hydroxy-pentanoic acid, (R)-5-(4-{1-Ethyl-1-[4-(3-hydroxy-3-methyl-butyl)-3-methyl-phenyl]-propyl}-2-methyl-phenoxy)-4-hydroxy-pentanoic acid, (S)-6-(4-{1-Ethyl-1-[4-(3-hydroxy-3-methyl-butyl)-3-methyl-phenyl]-propyl}-2-methyl-phenoxy)-5-hydroxy-hexanoic acid, (R)-6-(4-{1-Ethyl-1-[4-(3-hydroxy-3-methyl-butyl)-3-methyl-phenyl]-propyl}-2-methyl-phenoxy)-5-hydroxy-hexanoic acid, (S)-3-(4-{1-Ethyl-1-[4-(3-hydroxy-3-methyl-butyl)-3-methyl-phenyl]-propyl}-2-methyl-phenoxy)-propane-1,2-diol, 2-(4-{1-Ethyl-1-[4-(3-hydroxy-3-methyl-butyl)-3-methyl-phenyl]-propyl}-2-methyl-phenoxymethyl)-propane-1,3-diol, 4-{1-Ethyl-1-[4-(3-hydroxy-3-methyl-butyl)-3-methyl-phenyl]-propyl}-2-methyl-phenol, 5-(4-{1-Ethyl-1-[4-(3-hydroxy-3-methyl-butyl)-3-methyl-phenyl]-propyl}-2-methyl-phenoxy)-pentanoic acid, 6-(4-{1-Ethyl-1-[4-(3-hydroxy-3-methyl-butyl)-3-methyl-phenyl]-propyl}-2-methyl-phenoxy)-hexanoic acid, 3-(4-{1-Ethyl-1-[4-(3-hydroxy-3-methyl-butyl)-3-methyl-phenyl]-propyl}-2-methyl-phenyl)-propionic acid, 4-(4-{1-[4-(5-Amino-pentyloxy)-3-methyl-phenyl]-1-ethyl-propyl}-2-methyl-phenyl)-2-methyl-butan-2-ol, 4-[4-(1-Ethyl-1-{3-methyl-4-[4-(1H-tetrazol-5-yl)-butoxy]-phenyl}-propyl)-2-methyl-phenyl]-2-methyl-butan-2-ol, (S)-5-(4-{1-Ethyl-1-[4-(1-hydroxy-cyclobutylethynyl)-3-methyl-phenyl]-propyl}-2-methyl-phenoxy)-4-hydroxy-pentanoic acid, (R)-5-(4-{1-Ethyl-1-[4-(1-hydroxy-cyclobutylethynyl)-3-methyl-phenyl]-propyl}-2-methyl-phenoxy)-4-hydroxy-pentanoic acid, (S)-6-(4-{1-Ethyl-1-[4-(1-hydroxy-cyclobutylethynyl)-3-methyl-phenyl]-propyl}-2-methyl-phenoxy)-5-hydroxy-hexanoic acid, (R)-6-(4-{1-Ethyl-1-[4-(1-hydroxy-cyclobutylethynyl)-3-methyl-phenyl]-propyl}-2-methyl-phenoxy)-5-hydroxy-hexanoic acid, (S)-3-(4-{1-Ethyl-1-[4-(1-hydroxy-cyclobutylethynyl)-3-methyl-phenyl]-propyl}-2-methyl-phenoxy)-propane-1,2-diol, 2-(4-{1-Ethyl-1-[4-(1-hydroxy-cyclobutylethynyl)-3-methyl-phenyl]-propyl}-2-methyl-phenoxymethyl)-propane-1,3-diol, 4-{1-Ethyl-1-[4-(1-hydroxy-cyclobutylethynyl)-3-methyl-phenyl]-propyl}-2-methyl-phenol, 5-(4-{1-Ethyl-1-[4-(1-hydroxy-cyclobutylethynyl)-3-methyl-phenyl]-propyl}-2-methyl-phenoxy)-pentanoic acid, 6-(4-{1-Ethyl-1-[4-(1-hydroxy-cyclobutylethynyl)-3-methyl-phenyl]-propyl}-2-methyl-phenoxy)-hexanoic acid, 3-(4-{1-Ethyl-1-[4-(1-hydroxy-cyclobutylethynyl)-3-methyl-phenyl]-propyl}-2-methyl-phenyl)-propionic acid, 1-(4-{1-[4-(5-Amino-pentyloxy)-3-methyl-phenyl]-l -ethyl-propyl}-2-methyl-phenylethynyl)-cyclobutanol, 1-[4-(1-Ethyl-1-{3-methyl-4-[4-(1H-tetrazol-5-yl)-butoxy]-phenyl}-propyl)-2-methyl-phenylethynyl]-cyclobutanol, (S)-5-[4-(1-Ethyl-1-{4-[(E)-2-(1-hydroxy-cyclobutyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclobutyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclobutyl)-vinyl]-3-methyl-phenyl}-propyl)-2-methyl-phenoxy]-5-hydroxy-hexanoic acid, (R)-6-[4-(1-Ethyl-1-{4-[(E)-2-(1-hydroxy-cyclobutyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclobutyl)-vinyl]-3-methyl-phenyl}-propyl)-2-methyl-phenoxy]-propane-1,2-diol, 2-[4-(1-Ethyl-1-{4-[(E)-2-(1-hydroxy-cyclobutyl)-vinyl]-3-methyl-phenyl}-propyl)-2-methyl-phenoxymethyl]-propane-1,3-diol, 4-(1-Ethyl-1-{4-[(E)-2-(1-hydroxy-cyclobutyl)-vinyl]-3-methyl-phenyl}-propyl)-2-methyl-phenol, 5-[4-(1-Ethyl-1-{4-[(E)-2-(1-hydroxy-cyclobutyl)-vinyl]-3-methyl-phenyl}-propyl)-2-methyl-phenoxy]-pentanoic acid, 6-[4-(1-Ethyl-1-{4-[(E)-2-(1-hydroxy-cyclobutyl)-vinyl]-3-methyl-phenyl}-propyl)-2-methyl-phenoxy]-hexanoic acid, 3-[4-(1-Ethyl-1-{4-[(E)-2-(1-hydroxy-cyclobutyl)-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclobutanol, 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclobutanol, (S)-5-[4-(1-Ethyl-1-{4-[2-(1-hydroxy-cyclobutyl)-ethyl]-3-methyl-phenyl}-propyl)-2-methyl-phenoxy]-4-hydroxy-pentanoic acid, (R)-5-[4-(1-Ethyl-1-{4-[2-(1-hydroxy-cyclobutyl)-ethyl]-3-methyl-phenyl}-propyl)-2-methyl-phenoxy]-4-hydroxy-pentanoic acid, (S)-6-[4-(1-Ethyl-1-{4-[2-(1-hydroxy-cyclobutyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclobutyl)-ethyl]-3-methyl-phenyl}-propyl)-2-methyl-phenoxy]-5-hydroxy-hexanoic acid, (S)-3-[4-(1-Ethyl-1-{4-[2-(1-hydroxy-cyclobutyl)-ethyl]-3-methyl-phenyl}-propyl)-2-methyl-phenoxy]-propane-1,2-diol, 2-[4-(1-Ethyl-1-{4-[2-(1-hydroxy-cyclobutyl)-ethyl]-3-methyl-phenyl}-propyl)-2-methyl-phenoxymethyl]-propane-1,3-diol, 4-(1-Ethyl-1-{4-[2-(1-hydroxy-cyclobutyl)-ethyl]-3-methyl-phenyl}-propyl)-2-methyl-phenol, 5-[4-(1-Ethyl-1-{4-[2-(1-hydroxy-cyclobutyl)-ethyl]-3-methyl-phenyl}-propyl)-2-methyl-phenoxy]-pentanoic acid, 6-[4-(1-Ethyl-1-{4-[2-(1-hydroxy-cyclobutyl)-ethyl]-3-methyl-phenyl}-propyl)-2-methyl-phenoxy]-hexanoic acid, 3-[4-(1-Ethyl-1-{4-[2-(1-hydroxy-cyclobutyl)-ethyl]-3-methyl-phenyl}-propyl)-2-methyl-phenyl]-propionic acid, 1-[2-(4-{1-[4-(5-Amino-pentyloxy)-3-methyl-phenyl]-1-ethyl-propyl}-2-methyl-phenyl)-ethyl]-cyclobutanol, 1-{2-[4-(1-Ethyl-1-{3-methyl-4-[4-(1H-tetrazol-5-yl)-butoxy]-phenyl}-propyl)-2-methyl-phenyl]-ethyl}-cyclobutanol, (S)-5-(4-{1-Ethyl-1-[4-(1-hydroxy-cyclopentylethynyl)-3-methyl-phenyl]-propyl}-2-methyl-phenoxy)-4-hydroxy-pentanoic acid, (R)-5-(4-{1-Ethyl-1-[4-(1-hydroxy-cyclopentylethynyl)-3-methyl-phenyl]-propyl}-2-methyl-phenoxy)-4-hydroxy-pentanoic acid, (S)-6-(4-{1-Ethyl-1-[4-(1-hydroxy-cyclopentylethynyl)-3-methyl-phenyl]-propyl}-2-methyl-phenoxy)-5-hydroxy-hexanoic acid, (R)-6-(4-{1-Ethyl-1-[4-(1-hydroxy-cyclopentylethynyl)-3-methyl-phenyl]-propyl}-2-methyl-phenoxy)-5-hydroxy-hexanoic acid, (S)-3-(4-{1-Ethyl-1-[4-(1-hydroxy-cyclopentylethynyl)-3-methyl-phenyl]-propyl}-2-methyl-phenoxy)-propane-1,2-diol, 2-(4-{1-Ethyl-1-[4-(1-hydroxy-cyclopentylethynyl)-3-methyl-phenyl]-propyl}-2-methyl-phenoxymethyl)-propane-1,3-diol, 4-}1-Ethyl-1-[4-(1-hydroxy-cyclopentylethynyl)-3-methyl-phenyl]-propyl}-2-methyl-phenol, 5-(4-{1-Ethyl-1-[4-(1-hydroxy-cyclopentylethynyl)-3-methyl-phenyl]-propyl}-2-methyl-phenoxy)-pentanoic acid, 6-(4-{1-Ethyl-1-[4-(1-hydroxy-cyclopentylethynyl)-3-methyl-phenyl]-propyl}-2-methyl-phenoxy)-hexanoic acid, 3-(4-{1-Ethyl-1-[4-(1-hydroxy-cyclopentylethynyl)-3-methyl-phenyl]-propyl}-2-methyl-phenyl)-propionic acid, 1-(4-{1-[4-(5-Amino-pentyloxy)-3-methyl-phenyl]-1-ethyl-propyl)-2-methyl-phenylethynyl)-cyclopentanol, 1-[4-(1-Ethyl-1-{3-methyl-4-[4-(1H-tetrazol-5-yl)-butoxy]-phenyl}-propyl)-2-methyl-phenylethynyl]-cyclopentanol, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclopentyl)-vinyl]-3-methyl-phenyl}-propyl)-2-methyl-phenoxy]-5-hydroxy-hexanoic acid, (R)-6-[4-(1-Ethyl-1-{4-[(E)-2-(1-hydroxy-cyclopentyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclopentyl)-vinyl]-3-methyl-phenyl}-propyl)-2-methyl-phenoxy]-propane-1,2-diol, 2-[4-(1-Ethyl-1-{4-[(E)-2-(1-hydroxy-cyclopentyl)-vinyl]-3-methyl-phenyl}-propyl)-2-methyl-phenoxymethyl]-propane-1,3-diol, 4-(1-Ethyl-1-{4-[(E)-2-(1-hydroxy-cyclopentyl)-vinyl]-3-methyl-phenyl}-propyl)-2-methyl-phenol, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 6-[4-(1-Ethyl-1-{4-[(E)-2-(1-hydroxy-cyclopentyl)-vinyl]-3-methyl-phenyl}-propyl)-2-methyl-phenoxy]-hexanoic acid, 3-[4-(1-Ethyl-1-{4-[(E)-2-(1-hydroxy-cyclopentyl)-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclopentanol, 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclopentanol, (S)-5-[4-(1-Ethyl-1-{4-[2-(1-hydroxy-cyclopentyl)-ethyl]-3-methyl-phenyl}-propyl)-2-methyl-phenoxy]-4-hydroxy-pentanoic acid, (R)-5-[4-(1-Ethyl-1-{4-[2-(1-hydroxy-cyclopentyl)-ethyl]-3-methyl-phenyl-propyl)-2-methyl-phenoxy]-4-hydroxy-pentanoic acid, (S)-6-[4-(1-Ethyl-1-{4-[2-(1-hydroxy-cyclopentyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclopentyl)-ethyl]-3-methyl-phenyl}-propyl)-2-methyl-phenoxy]-5-hydroxy-hexanoic acid, (S)-3-[4-(1-Ethyl-1-{4-[2-(1-hydroxy-cyclopentyl)-ethyl]-3-methyl-phenyl}-propyl)-2-methyl-phenoxy]-propane-1,2-diol, 2-[4-(1-Ethyl-1-{4-[2-(1-hydroxy-cyclopentyl)-ethyl]-3-methyl-phenyl}-propyl)-2-methyl-phenoxymethyl]-propane-1,3-diol, 4-(1-Ethyl-1-{4-[2-(1-hydroxy-cyclopentyl)-ethyl]-3-methyl-phenyl}-propyl)-2-methyl-phenol, 5-[4-(1-Ethyl-1-{4-[2-(1-hydroxy-cyclopentyl)-ethyl]-3-methyl-phenyl}-propyl)-2-methyl-phenoxy]-pentanoic acid, 6-[4-(1-Ethyl-1-{4-[2-(1-hydroxy-cyclopentyl)-ethyl]-3-methyl-phenyl}-propyl)-2-methyl-phenoxy]-hexanoic acid, 3-[4-(1-Ethyl-1-{4-[2-(1-hydroxy-cyclopentyl)-ethyl]-3-methyl-phenyl-propyl)-2-methyl-phenyl]-propionic acid, 1-[2-(4-{1-[4-(5-Amino-pentyloxy)-3-methyl-phenyl]-1-ethyl-propyl}-2-methyl-phenyl)-ethyl]-cyclopentanol, 1-{2-[4-(1-Ethyl-1-{3-methyl-4-[4-(1H-tetrazol-5-yl)-butoxy]-phenyl}-propyl)-2-methyl-phenyl]-ethyl}-cyclopentanol, (S)-5-(4-{1-Ethyl-1-[4-(1-hydroxy-cyclohexylethynyl)-3-methyl-phenyl]-propyl}-2-methyl-phenoxy)-4-hydroxy-pentanoic acid, (R)-5-(4-{1-Ethyl-1-[4-(1-hydroxy-cyclohexylethynyl)-3-methyl-phenyl]-propyl}-2-methyl-phenoxy)-4-hydroxy-pentanoic acid, (S)-6-(4-{1-Ethyl-1-[4-(1-hydroxy-cyclohexylethynyl)-3-methyl-phenyl]-propyl}-2-methyl-phenoxy)-5-hydroxy-hexanoic acid, (R)-6-(4-{1-Ethyl-1-[4-(1-hydroxy-cyclohexylethynyl)-3-methyl-phenyl]-propyl}-2-methyl-phenoxy)-5-hydroxy-hexanoic acid, (S)-3-(4-{1-Ethyl-1-[4-(1-hydroxy-cyclohexylethynyl)-3-methyl-phenyl]-propyl-2-methyl-phenoxy)-propane-1,2-diol, 2-(4-{1-Ethyl-1-[4-(1-hydroxy-cyclohexylethynyl)-3-methyl-phenyl]-propyl}-2-methyl-phenoxymethyl)-propane-1,3-diol, 4-{1-Ethyl-1-[4-(1-hydroxy-cyclohexylethynyl)-3-methyl-phenyl]-propyl)-2-methyl-phenol, 5-(4-{1-Ethyl-1-[4-(-hydroxy-cyclohexylethynyl)-3-methyl-phenyl]-propyl-2-methyl-phenoxy)-pentanoic acid, 6-(4-{1-Ethyl-1-[4-(1-hydroxy-cyclohexylethynyl)-3-methyl-phenyl]-propyl}-2-methyl-phenoxy)-hexanoic acid, 3-(4-{1-Ethyl-1-[4-(1-hydroxy-cyclohexylethynyl)-3-methyl-phenyl]-propyl)-2-methyl-phenyl)-propionic acid, 1-(4-{1-[4-(5-Amino-pentyloxy)-3-methyl-phenyl]-1-ethyl-propyl}-2-methyl-phenylethynyl)-cyclohexanol, 1-[4-(1-Ethyl-1-{3-methyl-4-[4-(1H-tetrazol-5-yl)-butoxy]-phenyl}-propyl)-2-methyl-phenylethynyl]-cyclohexanol, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclohexyl)-vinyl]-3-methyl-phenyl}-propyl)-2-methyl-phenoxy]-5-hydroxy-hexanoic acid, (R)-6-[4-(1-Ethyl-1-{4-[(E)-2-(1-hydroxy-cyclohexyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclohexyl)-vinyl]-3-methyl-phenyl}-propyl)-2-methyl-phenoxy]-propane-1,2-diol, 2-[4-(1-Ethyl-1-{4-[(E)-2-(1-hydroxy-cyclohexyl)-vinyl]-3-methyl-phenyl}-propyl)-2-methyl-phenoxymethyl]-propane-1,3-diol, 4-(1-Ethyl-1-{4-[(E)-2-(1-hydroxy-cyclohexyl)-vinyl]-3-methyl-phenyl}-propyl)-2-methyl-phenol, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-[4-(1-Ethyl-1-{4-[(E)-2-(1-hydroxy-cyclohexyl)-vinyl]-3-methyl-phenyl)-propyl)-2-methyl-phenoxy]-hexanoic acid, 3-[4-(1-Ethyl-1-{4-[(E)-2-(1-hydroxy-cyclohexyl)-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclohexanol, 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclohexanol, (S)-5-[4-(1-Ethyl-1-{4-[2-(1-hydroxy-cyclohexyl)-ethyl]-3-methyl-phenyl}-propyl)-2-methyl-phenoxy]-4-hydroxy-pentanoic acid, (R)-5-[4-(1-Ethyl-1-{4-[2-(1-hydroxy-cyclohexyl)-ethyl]-3-methyl-phenyl}-propyl)-2-methyl-phenoxy]-4-hydroxy-pentanoic acid, (S)-6-[4-(1-Ethyl-1-{4-[2-(1-hydroxy-cyclohexyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclohexyl)-ethyl]-3-methyl-phenyl}-propyl)-2-methyl-phenoxy]-5-hydroxy-hexanoic acid, (S)-3-[4-(1-Ethyl-1-{4-[2-(1-hydroxy-cyclohexyl)-ethyl]-3-methyl-phenyl}-propyl)-2-methyl-phenoxy]-propane-1,2-diol, 2-[4-(1-Ethyl-1-{4-[2-(1-hydroxy-cyclohexyl)-ethyl]-3-methyl-phenyl}-propyl)-2-methyl-phenoxymethyl]-propane-1,3-diol, 4-(1-Ethyl-1-{4-[2-(1-hydroxy-cyclohexyl)-ethyl]-3-methyl-phenyl}-propyl)-2-methyl-phenol, 5-[4-(1-Ethyl-1-{4-[2-(1-hydroxy-cyclohexyl)-ethyl]-3-methyl-phenyl}-propyl)-2-methyl-phenoxy]-pentanoic acid, 6-[4-(1-Ethyl-1-{4-[2-(1-hydroxy-cyclohexyl)-ethyl]-3-methyl-phenyl}-propyl)-2-methyl-phenoxy]-hexanoic acid, 3-[4-(1-Ethyl-1-{4-[2-(1-hydroxy-cyclohexyl)-ethyl]-3-methyl-phenyl}-propyl)-2-methyl-phenyl]-propionic acid, 1-[2-(4-{1-[4-(5-Amino-pentyloxy)-3-methyl-phenyl]-1-ethyl-propyl}-2-methyl-phenyl)-ethyl]-cyclohexanol, 1-{2-[4-(1-Ethyl-1-{3-methyl-4-[4-(1H-tetrazol-5-yl)-butoxy]-phenyl}-propyl)-2-methyl-phenyl]-ethyl}-cyclohexanol, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (R)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-6-(4-{1-Ethyl-1-[4-(3-hydroxy-4,4-dimethyl-pent-1-ynyl)-3-methyl-phenyl]-propyl}-2-methyl-phenoxy)-5-hydroxy-hexanoic acid, (R)-6-(4-{1-Ethyl-1-[4-(3-hydroxy-4,4-dimethyl-pent-1-ynyl)-3-methyl-phenyl]-propyl}-2-methyl-phenoxy)-5-hydroxy-hexanoic acid, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, 2-(4-{1-Ethyl-1-[4-(3-hydroxy-4,4-dimethyl-pent-1-ynyl)-3-methyl-phenyl]-propyl}-2-methyl-phenoxymethyl)-propane-1,3-diol, 4-{1-Ethyl-1-[4-(3-hydroxy-4,4-dimethyl-pent-1-ynyl)-3-methyl-phenyl]-propyl}-2-methyl-phenol, 5-(4-{1-Ethyl-1-[4-(3-hydroxy-4,4-dimethyl-pent-1-ynyl)-3-methyl-phenyl]-propyl}-2-methyl-phenoxy)-pentanoic acid, 6-(4-{1-Ethyl-1-[4-(3-hydroxy-4,4-dimethyl-pent-1-ynyl)-3-methyl-phenyl]-propyl}-2-methyl-phenoxy)-hexanoic acid, 3-(4-{1-Ethyl-1-[4-(3-hydroxy-4,4-dimethyl-pent-1-ynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-4,4-dimethyl-pent-1-yn-3-ol, 1-[4-(1-Ethyl-1-{3-methyl-4-[4-(1H-tetrazol-5-yl)-butoxy]-phenyl}-propyl)-2-methyl-phenyl]-4,4-dimethyl-pent-1-yn-3-ol, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (R)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-6-(4-{1-Ethyl-1-[4-((E)-3-hydroxy-4,4-dimethyl-pent-1-enyl)-3-methyl-phenyl]-propyl}-2-methyl-phenoxy)-5-hydroxy-hexanoic acid, (R)-6-(4-{1-Ethyl-1-[4-((E)-3-hydroxy-4,4-dimethyl-pent-1-enyl)-3-methyl-phenyl]-propyl}-2-methyl-phenoxy)-5-hydroxy-hexanoic acid, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 4-{1-Ethyl-1-[4-((E)-3-hydroxy-4,4-dimethyl-pent-1-enyl)-3-methyl-phenyl]-propyl}-2-methyl-phenol, 5-(4-{1-Ethyl-1-[4-((E)-3-hydroxy-4,4-dimethyl-pent-1-enyl)-3-methyl-phenyl]-propyl}-2-methyl-phenoxy)-pentanoic acid, 6-(4-{1-Ethyl-1-[4-((E)-3-hydroxy-4,4-dimethyl-pent-1-enyl)-3-methyl-phenyl]-propyl}-2-methyl-phenoxy)-hexanoic acid, 3-(4-{1-Ethyl-1-[4-((E)-3-hydroxy-4,4-dimethyl-pent-1-enyl)-3-methyl-phenyl]-propyl}-2-methyl-phenyl)-propionic acid, (E)-1-(4-{1-[4-(5-Amino-pentyloxy)-3-methyl-phenyl]-1-ethyl-propyl}-2-methyl-phenyl)-4,4-dimethyl-pent-1-en-3-ol, (E)-1-[4-(1-Ethyl-1-{3-methyl-4-[4-(1H-tetrazol-5-yl)-butoxy]-phenyl}-propyl)-2-methyl-phenyl]-4,4-dimethyl-pent-1-en-3-ol, (S)-5-(4-{1-Ethyl-1-[4-(3-hydroxy-4,4-dimethyl-pentyl)-3-methyl-phenyl]-propyl}-2-methyl-phenoxy)-4-hydroxy-pentanoic acid, (R)-5-(4-{1-Ethyl-1-[4-(3-hydroxy-4,4-dimethyl-pentyl)-3-methyl-phenyl]-propyl}-2-methyl-phenoxy)-4-hydroxy-pentanoic acid, (S)-6-(4-{1-Ethyl-1-[4-(3-hydroxy-4,4-dimethyl-pentyl)-3-methyl-phenyl]-propyl}-2-methyl-phenoxy)-5-hydroxy-hexanoic acid, (R)-6-(4-{1-Ethyl-1-[4-(3-hydroxy-4,4-dimethyl-pentyl)-3-methyl-phenyl]-propyl}-2-methyl-phenoxy)-5-hydroxy-hexanoic acid, (S)-3-(4-{1-Ethyl-1-[4-(3-hydroxy-4,4-dimethyl-pentyl)-3-methyl-phenyl]-propyl}-2-methyl-phenoxy)-propane-1,2-diol, 2-(4-{1-Ethyl-1-[4-(3-hydroxy-4,4-dimethyl-pentyl)-3-methyl-phenyl]-propyl}-2-methyl-phenoxymethyl)-propane-1,3-diol, 4-{1-Ethyl-1-[4-(3-hydroxy-4,4-dimethyl-pentyl)-3-methyl-phenyl]-propyl}-2-methyl-phenol, 5-(4-{1-Ethyl-1-[4-(3-hydroxy-4,4-dimethyl-pentyl)-3-methyl-phenyl]-propyl}-2-methyl-phenoxy)-pentanoic acid, 6-(4-{1-Ethyl-1-[4-(3-hydroxy-4,4-dimethyl-pentyl)-3-methyl-phenyl]-propyl}-2-methyl-phenoxy)-hexanoic acid, 3-(4-{1-Ethyl-1-[4-(3-hydroxy-4,4-dimethyl-pentyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-4,4-dimethyl-pentan-3-ol, 1-[4-(1-Ethyl-1-{3-methyl-4-[4-(1H-tetrazol-5-yl)-butoxy]-phenyl}-propyl)-2-methyl-phenyl]-4,4-dimethyl-pentan-3-ol, 4-(4-{1-Ethyl-1-[4-(3-hydroxy-4,4-dimethyl-pentyl)-3-methyl-phenyl]-propyl}-2-methyl-phenyl)-butane-1,2-diol, (S)-3-(4-{1-Ethyl-1-[4-((R)-3-hydroxy-4,4-dimethyl-pentyl)-3-methyl-phenyl]-propyl}-2-methyl-phenoxy)-propane-1,2-diol, (R)-3-(4-{1-Ethyl-1-[4-((R)-3-hydroxy-4,4-dimethyl-pentyl)-3-methyl-phenyl]-propyl}-2-methyl-phenoxy)-propane-1,2-diol, (S)-3-(4-{1-Ethyl-1-[4-((S)-3-hydroxy-4,4-dimethyl-pentyl)-3-methyl-phenyl]-propyl}-2-methyl-phenoxy)-propane-1,2-diol, (R)-3-(4-{1-Ethyl-1-[4-((S)-3-hydroxy-4,4-dimethyl-pentyl)-3-methyl-phenyl]-propyl}-2-methyl-phenoxy)-propane-1,2-diol, 2-(4-{1-Ethyl-1-[4-(3-hydroxy-4,4-dimethyl-pentyl)-3-methyl-phenyl]-propyl}-2-methyl-phenoxymethyl)-propane-1,3-diol, 3-(4-{1-Ethyl-1-[4-(3-hydroxy-4,4-dimethyl-pentyl)-3-methyl-phenyl]-propyl}-2-methyl-phenoxy)-propane-1,2-diol, (S)-5-(4-{1-Ethyl-1-[4-(3-hydroxy-4,4-dimethyl-pentyl)-3-methyl-phenyl]-propyl}-2-methyl-phenoxy)-pentane-1,4-diol, 1-(4-{1-Ethyl-1-[4-(3-hydroxy-4,4-dimethyl-pentyl)-3-methyl-phenyl]-propyl}-2-methyl-phenyl)-4,4-dimethyl-pentan-3-ol, 1-(4-{1-Ethyl-1-[4-(3-hydroxy-propyl)-3-methyl-phenyl]-propyl-2-methyl-phenyl)-4,4-dimethyl-pentan-3-ol, (S)-3-(4-{1-Ethyl-1-[4-(3-hydroxy-4,4-dimethyl-pentyl)-3-methyl-phenyl]-propyl}-2-methyl-phenoxy)-propane-1,2-diol, (R)-3-(4-{1-Ethyl-1-[4-(3-hydroxy-4,4-dimethyl-pentyl)-3-methyl-phenyl]-propyl}-2-methyl-phenoxy)-propane-1,2-diol, 4-(4-{1-Ethyl-1-[4-(2-hydroxy-3,3-dimethyl-butoxy)-3-methyl-phenyl]-propyl}-2-methyl-phenyl)-butane-1,2-diol, 1-(4-{1-Ethyl-1-[4-(3-hydroxy-propyl)-3-methyl-phenyl]-propyl}-2-methyl-phenoxy)-3,3-dimethyl-butan-2-ol, 3-(4-{1-Ethyl-1-[4-(2-hydroxy-3,3-dimethyl-butoxy)-3-methyl-phenyl]-propyl}-2-methyl-phenyl)-propane-1,2-diol, 3-(4-{1-Ethyl-1-[4-(2-hydroxy-3,3-dimethyl-butoxy)-3-methyl-phenyl]-propyl}-2-methyl-phenyl)-butane-1,2-diol, 1-(4-{1-Ethyl-1-[4-(2-hydroxy-3,3-dimethyl-butoxy)-3-methyl-phenyl]-propyl}-2-methyl-phenyl)-propane-1,2-diol, 1-(4-{1-Ethyl-1-[4-(3-hydroxy-butyl)-3-methyl-phenyl]-propyl}-2-methyl-phenoxy)-3,3-dimethyl-butan-2-ol, (E)-4-(4-{1-Ethyl-1-[4-(2-hydroxy-3,3-dimethyl-butoxy)-3-methyl-phenyl]-propyl}-2-methyl-phenyl)-but-3-en-2-ol, 3-(4-{1-Ethyl-1-[4-(2-hydroxy-3,3-dimethyl-butoxy)-3-methyl-phenyl]-propyl}-2-methyl-phenyl)-butan-1-ol, 1-(4-{1-[4-(2,3-Dihydroxy-propyl)-3-methyl-phenyl]-1-ethyl-propyl}-2-methyl-phenoxy)-3,3-dimethyl-butan-2-one, 1-(4-{1-[4-(2,3-Dihydroxy-1-methyl-propyl)-3-methyl-phenyl]-1-ethyl-propyl}-2-methyl-phenoxy)-3,3-dimethyl-butan-2-one, 1-(4-{1-Ethyl-1-[4-(3-hydroxy-butyl)-3-methyl-phenyl]-propyl}-2-methyl-phenoxy)-3,3-dimethyl-butan-2-one, 3-(4-{1-Ethyl-1-[4-(2-hydroxy-3,3-dimethyl-butyl)-3-methyl-phenyl]-propyl}-2-methyl-phenoxy)-propane-1,2-diol, (E)-1-(4-{1-[4-((S)-2,3-Dihydroxy-propoxy)-3-methyl-phenyl]-1-ethyl-propyl}-2-methyl-phenyl)-4,4-dimethyl-pent-1-en-3-one, (E)-1-(4-{1-[4-((R)-2,3-Dihydroxy-propoxy)-3-methyl-phenyl]-1-ethyl-propyl}-2-methyl-phenyl)-4,4-dimethyl-pent-1-en-3-one, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (R)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, 1-(4-{1-[4-((R)-2,3-Dihydroxy-propoxy)-3-methyl-phenyl]-1-ethyl-propyl}-2-methyl-phenyl)-4,4-dimethyl-pentan-3-one.

(69) The compound of the above fomula I which is (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid.

(70) The compound of the above fomula I which is (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid.

Another examples of the preferred compounds of the present invention are represented as follows.

(71) The compound of the above fomula I wherein Y is a substituent represented by following formula:

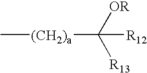

and R is a protecting group for a hydroxyl group.

(72) The compound according to (71) wherein

X is an optionally substituted methylene, an optionally substituted ethylene, an optionally substituted vinylene or an ethynylene;

R is a protecting group for a hydroxyl group selected from the group consisting of a methoxymethyl group, a methylthiomethyl group, a (phenyldimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group, a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-triflluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyldimethylsilyl group, a t-buthyidiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group;

R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;

W is a substituent represented by following formula:

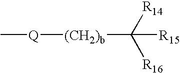

Q is —O—, a methylene, an ethylene, a vinylene, an ethynylene, —(CH2)ₖ-C(═O)NH— or —O—(CH2)ₖ-C(═O)NH—;

R14 is a hydrogen atom, a hydroxyl group, an optionally substituted carboxyl group, an optionally substituted carbamoyl group, an optionally substituted C1-6 alkyl group, an optionally substituted C3-C12 cycloalkyl group, an optionally substituted C6-C12 aryl group, an optionally substituted (C6-C12)aryl-(C1-4)alkyl group, —OR17 or —N(R18)R19;

R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group selected from the group consisting of an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group; an isoquinoline group, a cinnoline group, a phthalazine group, a quinazoline group, a quinoxaline group, or R15 and R16 may together form ═O;

R17 is an optionally substituted C1-6 alkyl group or an optionally substituted C3-C6 cycloalkyl group;

R18 and R19 are each independently selected from a hydrogen atom, an optionally substituted C1-6 alkyl group or an optionally substituted C3-C6 cycloalkyl group;

or one of (R15 and R17), (R16 and R17), (R15 and R18), (R16 and R18), (R15 and R19) or (R16 and R19) may together form a 3-12 membered cyclic ring which is selected from a lactam ring or a lactone ring;

R1 is a C1-6 alkyl group;

R2 is a C1-6 alkyl group;

R3 is a hydrogen atom or a C1-6 alkyl group;

R4 is a halogen atom or a C1-6 alkyl group;

R5 is a hydrogen atom;

R6 is a halogen atom or a C1-6 alkyl group.

(73) The compound according to (72) wherein

X is an ethylene, a vinylene, or an ethynylene;

R is a protecting group for a hydroxyl group selected from the group consisting of a methoxymethyl group, a 2-(trimethylsilyl)ethoxymethyl group, a tetrahydropyranyl group, a benzyl group, a p-methoxybenzyl group, a trimethylsilyl group, a triethylsilyl group, a t-butyldimethylsilyl group, a t-buthyidiphenylsilyl group, an acetyl group, a pivaloyl group, a benzoyl group, a methanesulfonyl group, a tosyl group, a trifluoromethanesulfonyl group;

R12 and R13 are each independently selected from the group consisting of a hydrogen atom, a C1-8 alkyl group which may be substituted with a halogen atom(s) a C3-8 cycloalkyl group which may be substituted with a C1-4 alkyl group, or R12 and R13 are together form a C3-8 cycloalkyl group which may be substituted with a C1-4 alkyl group;

W is a substituent represented by following formula:

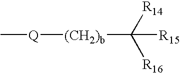

Q is —O—, a methylene, an ethylene, a vinylene, an ethynylene, —(CH2)ₖ-C(═O)NH— or —O—(CH2)ₖ-C(═O)NH—;

b is an integer of 0 to 5;

R14 is a hydrogen atom, a hydroxyl group, a carboxyl group which may be substituted with a C1-4 alkyl group, a carbamoyl group which may be substituted with a C1-4 alkyl group, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, OR17 or —N(R18)R19;

R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, a 3-8 membered heterocyclic group which may be substituted with a C1-4 alkyl group, a hydroxyl group, a carboxyl group, a carbamoyl group or an amino group, wherein said heterocyclic group selected from the group consisting of an oxetane group, a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a pyridine group, a pyridazine group, a pyrimidine group, a pyrazine group, a tetrahydropyran group, a pyran group, a thiopyran group, or R15 and R16 may together form ═O;

and at least one of R14, R15 or R16 is a hydrogen atom;

R17 is a C1-4 alkyl group;

R18 and R19 are each independently selected from a hydrogen atom or a C1-4 alkyl group;

or one of (R15 and R17), (R16 and R17), (R15 and R18), (R16 and R18), (R15 and R19) or (R16 and R19) may together form a 3-12 membered lactone ring.

(74) The compound according to (73) wherein

R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;

W is a substituent represented by following formula:

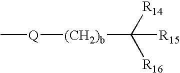

Q is —O—, —(CH2)ₖ-C(═O)NH— or —O—(CH2)ₖ-C(═O)NH—;

b is 0, 1 or 2;

k is 1;

R14 is a hydrogen atom, a hydroxyl group, a carboxyl group, a carbamoyl group, a C1-6 alkyl group which may be substituted with a hydroxyl group, a carboxyl group, a carbamoyl group or an amide group, a C3-C8 cycloalkyl group, a phenyl group which may be substituted with a hydroxyl group or a carboxyl group, a benzyl group which may be substituted with a hydroxyl group or a carboxyl group, —OR17 or —N(R18)R19;

R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, a 3-8 membered heterocyclic group selected from the group consisting of a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a pyridine group, a tetrahydropyran group;

and at least one of R14, R15 or R16 is a hydrogen atom;

R18 is a hydrogen atom;

R19 is a hydrogen atom;

or one of (R15 and R17), (R16 and R17), (R15 and R18), (R16 and R18), (R15 and R19),or (R16 and R19) may together form a 3-12 membered lactone ring;

R1 is a C1-4 alkyl group;

R2 is a C1-4 alkyl group;

R3 is a hydrogen atom or a C1-4 alkyl group;

R4 is a halogen atom or a C1-4 alkyl group;

R5 is a hydrogen atom;

R6 is a halogen atom or a C1-4 alkyl group.

(75) The compound according to (74) wherein

R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),

or R12 and R13 are together form a C3-10 cycloalkyl group;

Q is —O— or —O—(CH2)ₖ-C(═O)NH—;

R1 is an ethyl group;

R2 is an ethyl group;

R3 is a hydrogen atom or a methyl group;

R4 is a chlorine atom or a methyl group;

R6 is a chlorine atom, a methyl group, an ethyl group, a propyl group or an isopropyl group.

(76) The compound according to (74) wherein

R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),

or R12 and R13 are together form a C3-10 cycloalkyl group;

Q is a methylene, an ethylene, an ethynylene or —(CH2)ₖ-C(═O)NH—;

R1 is an ethyl group;

R2 is an ethyl group;

R3 is a hydrogen atom;

R4 is a chlorine atom or a methyl group;

R6 is a chlorine atom or a methyl group.

(77) The compound according to (75) or (76) wherein R3 is a hydrogen atom.

(78) The compound according to (75) or (76) wherein R4 is a chlorine atom.

(79) The compound according to (75) or (76) wherein R6 is a chlorine atom.

(80) The compound according to (75) or (76) wherein R6 is a methyl group.

(81) The compound according to (75) or (76) wherein X is an ethylene.

(82) The compound according to (75) or (76) wherein X is a vinylene.

(83) The compound according to (75) or (76) wherein X is an ethynylene.

(84) The compound according to (75) or (76) wherein one of R12 and R13 is a hydrogen atom and the other is a C1-6 alkyl group.

(85) The compound according to (84) wherein one of R12 and R13 is a hydrogen atom and the other is a tert-butyl group.

(86) The compound according to (75) or (76) wherein both of R12 and R13 are same and a C1-6 alkyl group which may be substituted with a halogen atom(s).

(87) The compound according to (86) wherein R12 is a trifluoromethyl group and R13 is a trifluoromethyl group.

(88) The compound according to (75) wherein Q is —O—.

(89) The compound according to (76) wherein Q is an ethylene.

(90) The compound according to (76) wherein Q is an ethynylene.

(91) The compound according to (75) or (76) wherein at least one of R14, R15 or R16 is a substituent which have a hydroxyl group, a carboxyl group, a carbamoyl group or an amino group.

(92) The compound according to (75) or (76) wherein R14 is a hydroxyl group.

(93) The compound according to (75) or (76) wherein one of R15 and R16 is a hydrogen atom and the other is a C1-6 alkyl group substituted with a carboxyl group.

(94) The compound according to (75) or (76) wherein one of (R15 and R17), (R16 and R17), (R15 and R18), (R16 and R18), (R15 and R19) or (R16 and R19) together form a 3-12 membered lactone ring.

Another examples of the preferred compounds of the present invention are represented as follows.

(95) The compound of the above fomula I wherein W is a hydroxyl group, a carboxyl group or a trifluoromethanesulfonyloxy group.

(96) The compound according to (95) wherein

X is an optionally substituted methylene, an optionally substituted ethylene, an optionally substituted vinylene or an ethynylene;

Y is COOR8, CON(R9)R10, S(O)mR11 or a substituent represented by following formula:

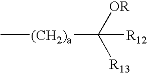

R is a hydrogen atom or a protecting group for a hydroxyl group selected from the group consisting of a methoxymethyl group, a methylthiomethyl group, a (phenyldimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group, a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-triflluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyidimethylsilyl group, a t-buthyidiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group;

R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;

R1 is a C1-6 alkyl group;

R2 is a C1-6 alkyl group;

R3 is a hydrogen atom or a C1-6 alkyl group;

R4 is a halogen atom or a C1-6 alkyl group;

R5 is a hydrogen atom;

R6 is a halogen atom or a C1-6 alkyl group.

(97) The compound according to (96) wherein

X is an ethylene, a vinylene, or an ethynylene;

Y is a substituent represented by following formula:

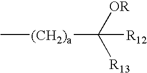

R is a hydrogen atom or a protecting group for a hydroxyl group selected from the group consisting of a methoxymethyl group, a 2-(trimethylsilyl)ethoxymethyl group, a tetrahydropyranyl group, a benzyl group, a p-methoxybenzyl group, a trimethylsilyl group, a triethylsilyl group, a t-butyldimethylsilyl group, a t-buthyldiphenylsilyl group, an acetyl group, a pivaloyl group, a benzoyl group, a methanesulfonyl group, a tosyl group, a trifluoromethanesulfonyl group;

R12 and R13 are each independently selected from the group consisting of a hydrogen atom, a C1-8 alkyl group which may be substituted with a halogen atom(s), a C3-8 cycloalkyl group which may be substituted with a C1-4 alkyl group, or R12 and R13 are together form a C3-8 cycloalkyl group which may be substituted with a C1-4 alkyl group.

(98) The compound according to (97) wherein

R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;

R1 is a C1-4 alkyl group;

R2 is a C1-4 alkyl group;

R3 is a hydrogen atom or a C1-4 alkyl group;

R4 is a halogen atom or a C1-4 alkyl group;

R5 is a hydrogen atom;

R6 is a halogen atom or a C1-4 alkyl group.

(99) The compound according to (98) wherein

R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),

or R12 and R13 are together form a C3-10 cycloalkyl group;

R1 is an ethyl group;

R2 is an ethyl group;

R3 is a hydrogen atom or a methyl group;

R4 is a chlorine atom or a methyl group;

R6 is a chlorine atom, a methyl group, an ethyl group, a propyl group or an isopropyl group.

(100) The compound according to (99) wherein W is a carboxyl group.

(101) The compound according to (100) which is 4-{1-Ethyl-1-[4-((E)-3-ethyl-3-hydroxy-pent-1-enyl)-3-methyl-phenyl]-propyl}-2-methyl-benzoic acid.

(102) The compound according to (99) or (100) wherein wherein R3 is a hydrogen atom.

(103) The compound according to (99) or (100) wherein R4 is a chlorine atom.

(104) The compound according to (99) or (100) wherein R6 is a chlorine atom.

(105) The compound according to (99) or (100) wherein R6 is a methyl group.

(106) The compound according to (99) or (100) wherein X is an ethylene.

(107) The compound according to (99) or (100) wherein X is a vinylene.

(108) The compound according to (99) or (100) wherein X is an ethynylene.

(109) The compound according to (99) or (100) wherein one of R12and R13 is a hydrogen atom and the other is a C1-6 alkyl group.

(110) The compound according to (109) wherein one of R12 and R13 is a hydrogen atom and the other is a tert-butyl group.

(111) The compound according to (99) or (100) wherein both of R12 and R13 are same and a C1-6 alkyl group which may be substituted with a halogen atom(s).

(112) The compound according to (111) wherein R12 is a trifluoromethyl group and R13 is a trifluoromethyl group.

Pharmaceutical Use

The compounds of the present invention exhibit VDR modulating activity. Therefore, the compounds and compositions of the present invention are useful as pharmaceuticals, such as, for the treatment of abscess, acne, adhesion, alopecia, Alzheimer's disease, benign prostatic hyperplasia, bone fracture healing, cancer, autoimmune induced diabetes, host-graft rejection, insufficient sebum secretion, insufficient dermal firmness, humoral hypercalcemia, insufficient dermal hydration, leukemia, lupus, multiple sclerosis, osteomalacia, osteoporosis, psoriaticarthritis, psoriasis, renal failure, renal osteodystrophy, rheumatoid arthritis, scleroderma, secondary hyperparathyroidism, systemic lupus erythematosus, and wrinkles, cornea wound, cornea healing, retinopathy, sway, muscle weakness, fall, chronic glomerulonephritis, lupus nephritis, diabetic nephropathy, hypocalcemia, hypoparathyroidism, rickets, osteoarthritis, and the like conditions and diseases. Especially, the compounds and compositions of the present invention are useful as pharmaceuticals for the treatment of benign prostatic hyperplasia, cancer, osteoporosis, psoriasis, secondary hyperparathyroidism, chronic glomerulonephritis, lupus nephritis, diabetic nephropathy, sway, muscle weakness, fall, rheumatoid arthritis and/or osteoarthritis. Among the above mentioned diseases, typical disease subjected to the treatment by the compounds and compositions of the present invention are benign prostatic hyperplasia, cancer, osteoporosis, psoriasis, secondary hyperparathyroidism, chronic glomerulonephritis, lupus nephritis and/or diabetic nephropathy. Certain compounds of the present invention are useful as a synthetic intermediate thereof.

Pharmaceutical Compositions

The compositions of the present invention comprise:.
(a) a safe and therapeutically effective amount of a compound of the present invention, or its corresponding enantiomer, diastereoisomer or tautomer, or pharmaceutically acceptable salt, or a prodrug thereof; and
(b) a pharmaceutically acceptable carrier.

The compounds useful in this invention can be formulated into pharmaceutical compositions for use in the treatment of numerous diseases as mentioned above, i.e. prophylaxis, management and treatment of these conditions. Standard pharmaceutical formulation techniques are used, such as those disclosed in Remington's Pharmaceutical Sciences, Mack Publishing Company, Easton, Pa.

A “safe and therapeutically effective amount” of a compound useful in the present invention is an amount that exhibits VDR modulating activity, in a subject, a tissue, or a cell, and preferably in an animal, more preferably in a mammal, without undue adverse side effects (such as toxicity, irritation, or allergic response), commensurate with a reasonable benefit/risk ratio, when used in the manner of this invention. The specific “safe and therapeutically effective amount” will vary with such factors as the particular condition being treated, the physical condition of the patient, the duration of treatment, the nature of concurrent therapy (if any), the specific dosage form to be used, the carrier employed, the solubility of the compound therein, and the dosage regimen desired for the composition.

In addition to the selected compound useful for the present invention, the compositions of the present invention contain a pharmaceutically-acceptable carrier. The term “pharmaceutically-acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

If the subject compound is to be injected, the preferred pharmaceutically-acceptable carrier is sterile, physiological saline, with blood-compatible suspending agent, the pH of which has been adjusted to about 7.4. In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parental administration include propylene glycol, ethyl oleate, pyrrolidone, ethanol, and sesame oil.

The compositions of this invention are preferably provided in unit dosage form. As used herein, a “unit dosage form” is a composition of this invention containing an amount of a compound that is suitable for administration to an animal, preferably mammal subject, in a single dose, according to good medical practice. (The preparation of a single or unit dosage form however, does not imply that the dosage form is administered once per day or once per course of therapy. Such dosage forms are contemplated to be administered once, twice, thrice or more per day, and are expected to be given more than once during a course of therapy, though a single administration is not specifically excluded. The skilled artisan will recognize that the formulation does not specifically contemplate the entire course of therapy and such decisions are left for those skilled in the art of treatment rather than formulation.)

The compositions useful for this invention may be in any of a variety of forms, suitable (for example) for oral, nasal, rectal, topical (including transdermal), ocular, intracereberally, intravenous, intramuscular, or parental administration. (The skilled artisan will appreciate that oral and nasal compositions comprise compositions that are administered by inhalation, and made using available methodologies.) Depending upon the particular route of administration desired, a variety of pharmaceutically-acceptable carriers well-known in the art may be used. These include solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrates such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions of the subject invention may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject in a treatment of dermatological conditions such as psoriasis, e.g., by the direct application or spreading of the composition on the epidermal or epithelial tissue of the subject, or transdermally via a “patch”. Such compositions include, for example, lotions, creams, solutions, gels and solids. These topical compositions preferably comprise a safe and effective amount. Suitable carriers for topical administration preferably remain in place on the skin as a continuous film, and resist being removed by perspiration or immersion in water. Generally, the carrier is organic in nature and capable of having dispersed or dissolved therein the compound. The carrier may include pharmaceutically-acceptable emollient, emulsifiers, thickening agents, solvents and the like.

Methods of Administration

The compounds and compositions useful in this invention can be administered topically or systemically. Systemic application includes any method of introducing compound into the tissues of the body, e.g., intra-articular, intrathecal, epidural, intramuscular, transdermal, intravenous, intraperitoneal, subcutaneous, sublingual administration, inhalation, rectal, or oral administration. The compounds useful in the present invention are preferably administered orally.

The specific dosage of the compound to be administered, as well as the duration of treatment is to be individualized by the treating clinicians. Typically, for a human adult, from about 1 ng/kg to 50 mg/kg, preferably from about 1 ng/kg to about 1 mg/kg, more preferably from about 10 ng/kg to about 100 μg/kg, of selected compound is administered per day. It is understood that these dosage ranges are by way of example only, and that daily administration can be adjusted depending on the factors i.e. type of disease, level of disease, ages of patients, sex of patients, route to be administered, etc.

In all of the foregoing, of course, the compounds useful in the present invention can be administered alone or as mixtures, and the compositions may further include additional drugs or excipients as appropriate for the indication.
General Synthetic Method

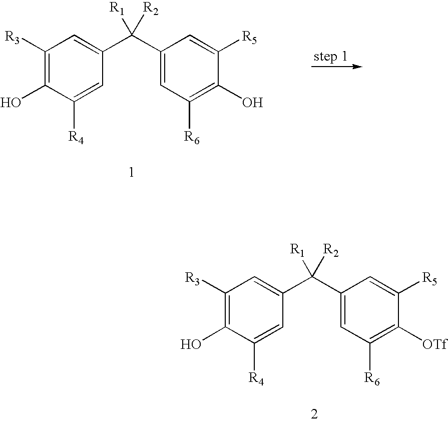

In the above scheme, the compound of general formula (1) obtained by the same procedure as described in WO00/10958 and the corresponding U.S. Pat. No. 6,218,430 B1 may be reacted with trifluoromethanesulfonic acid anhydride in the presence of a base to give a compound of general formula (2) (step 1).

Suitable bases for use in the above step 1 include pyridine, 2,6-lutidine, 2,4,6-collidine,N,N-dimethylaminopyridine, imidazole, triethylamine, more preferably pyridine.

Suitable solvents for use in the above step 1 include diethyl ether, tetrahydrofuran, dichloromethane, 1,2-dichloroethane, chloroform, benzene, toluene, more preferably dichloromethane.

The reaction temperature of the above step 1 is in the range from −50° C. to 50° C., preferably from −20° C. to 30° C., though it is not specifically limited so far as the reaction proceeds.

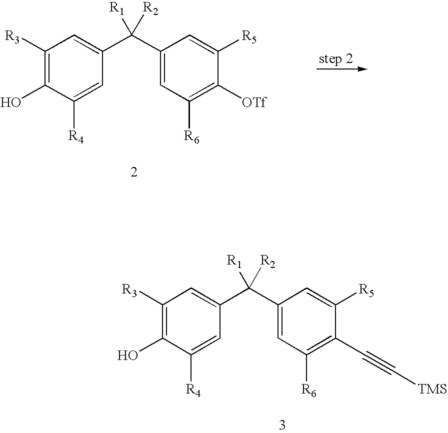

In the above scheme, the compound of general formula (2) may be reacted with trimethylsilyl acetylene in the presence of a palladium catalyst, copper (I) iodide, and triethyamine to give a compound of general formula (3) (step 2).

Suitable palladium catalysts for use in the above step 2 include tetrakis(triphenylphosphine)palladium, Pd(dba)2, Pd2(dba)3-CHCl3, palladium acetate, palladium chloride, [1,1′-bis(diphenylphosphino)-ferrocene]palladium dichloride dichloromethane complex. (dba; dibenzylideneacetone)

A ligand, such as triphenylphosphine, tributhylphosphine, tricyclohexylphosphine, 1,3-bis(diphenylphosphinopropane) or tri-t-buthyl phosphine, may be used in the above step 2 to increase the catalytic activity and/or the reaction selectivity.

Suitable solvents for use in the above step 2 include N,N-dimethylformamide, dimethylsulfoxide, N,N-dimethylacetamide, 1,3-dimethyl-2-imidazolidinone, tetrahydrofuran, toluene, acetonitrile, more preferably N,N-dimethylformamide or acetonitrile.

The reaction temperature of the above step 2 is in the range from 0° C. to 200° C., preferably from 20° C. to 150° C., though it is not specifically limited so far as the reaction proceeds.

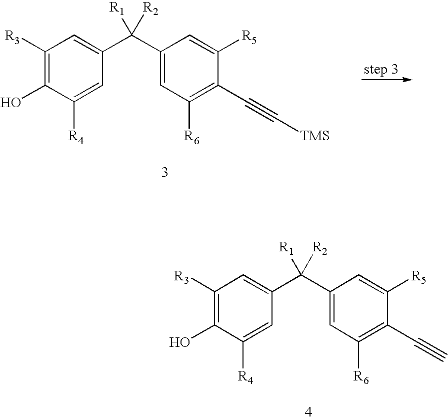

In the above scheme, the compound of general formula (3) may be reacted with tetra-n-butyl ammonium fluoride to give a compound of general formula (4) (step 3).

Suitable solvents for use in the above step 3 include N,N-dimethylformamide, dimethylsulfoxide, N,N-dimethylacetamide, 1,3-dimethyl-2-imidazolidinone, tetrahydrofuran, toluene, acetonitrile, more preferably tetrahydrofuran.

The reaction temperature of the above step 3 is in the range from 0° C. to 100° C., preferably from 0° C. to 50° C., though it is not specifically limited so far as the reaction proceeds.

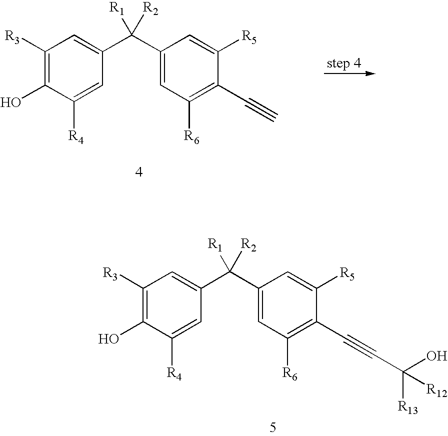

In the above scheme, the compound of general formula (4) may be reacted with a ketone or an aldehyde, described as R12(C═O)R13, in the presence of a base to give a compound of general formula (5) (step 4).

Suitable bases for use in the above step 4 include n-butyl lithium, sec-butyl lithium, tert-butyl lithium, methyl lithium, phenyl lithium, methyl magnesium bromide, methyl magnesium chloride, methyl magnesium iodide, isopropyl magnesium bromide, diisopropyl magnesium, lithium diisopropylamide, lithium bis(trimethylsilyl)amide, lithium 2,2,6,6-tetramethylpiperidide, lithium amide, sodium hydride, sodium bis(trimethylsilyl)amide, potassium hydride, potassium bis(trimethylsilyl) more preferably n-butyl lithium.

Suitable solvents for use in the above step 4 include hydrocarbon-ether-based solvents or the likes, such as pentane, hexane, benzene, toluene, diethyl ether, t-butyl methyl ether, tetrahydrofuran, 1,2-dimethoxyethane, 1,4-dioxane, anisole, more preferably tetrahydrofuran.

The reaction temperature of the above step 1 is in the range from −100° C. to 50° C., preferably from −80° C. to 30° C., though it is not specifically limited so far as the reaction proceeds.

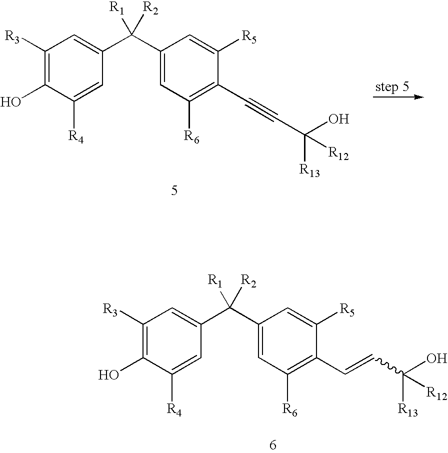

In the above scheme, the compound of general formula (5) may be reduced to give a compound of general formula (6) (step 5).

Step 5 includes the reduction by LiAlH4, Red-Al (sodium bis(2-methoxyethoxy)aluminium hydride) or hydrogenation using Lindlar catalyst.

Suitable solvents for use in the reduction by lithium aluminum hydride or Red-Al include hydrocarbon-ether-based solvents or the like, such as pentane, hexane, benzene, toluene, diethyl ether, t-butyl methyl ether, tetrahydrofuran, 1,2-dimethoxyethane, 1,4-dioxane, anisole, more preferably tetrahydrofuran.

Suitable solvents for use in the hydrogenation by Lindlar catalyst include methanol, ethanol, ethyl acetate, more preferably methanol.

The reaction temperature of the above step 5 is in the range from −50° C. to 200° C., preferably from 0° C. to 100° C., though it is not specifically limited so far as the reaction proceeds.

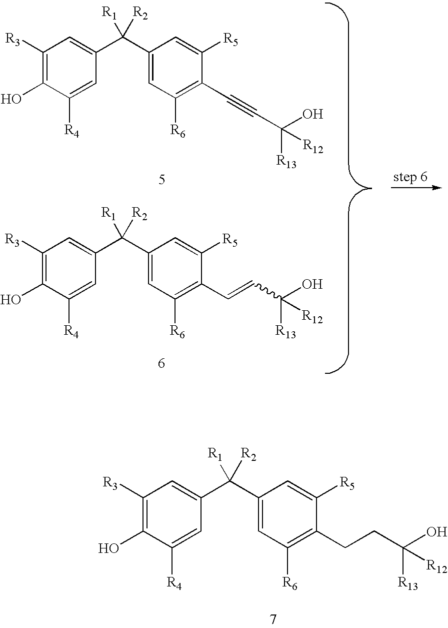

In the above scheme, the compound of general formula (5, 6) may be subjected to hydrogenation to give a compound of general formula (7) (step 6).

Suitable catalysts for use in the above step 6 include palladium-, rhodium-, ruthenium-, nickel-, platinum-based catalysts or the like, such as palladium on carbon, palladium hydroxide, platinum oxide, rhodium on alumina, Wilkinson's catalyst, more preferably palladium on carbon.

Suitable solvents for use in the above step 6 include methanol, ethanol, ethyl acetate, acetic acid, more preferably methanol.

The reaction temperature of the above step 6 is in the range from −50° C. to 200° C., preferably from 0° C. to 100° C., though it is not specifically limited so far as the reaction proceeds.

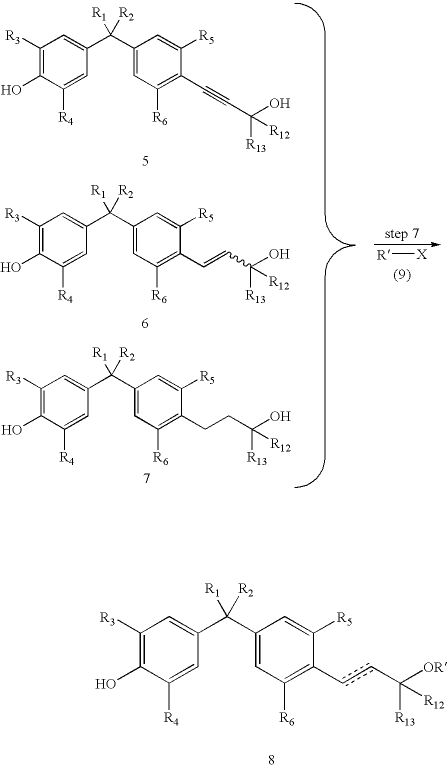

In the above scheme, the compound of general formula (5-7) may be subjected to protection of hydroxyl group with a compound of general formula (9) (R′=a methoxymethyl group, a 2-(trimethylsilyl)ethoxymethyl group, a benzyl group, a p-methoxybenzyl group, a trimethylsilyl group, a triethylsilyl group, a t-butyldimethylsilyl group, a t-buthyldiphenylsilyl group or an acetyl group; X=a halogen atom, a methanesulfonyloxy group, a toluenesulfonyloxy group, a trifluoromethanesulfonyloxy group or an acethyloxy group) in the presence of a base to give a compound of general formula (8) (step 7).

Suitable bases for use in the above step 7 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, pyridine, triethylamine, diisopropylethylamine, 2,6-lutidine, 2,4,6-collidine, N,N-dimethylaminopyridine, more preferably sodium hydride, potassium carbonate, or pyridine.

Suitable solvents for use in the above step 7 include dichloromethane, 1,2-dichloroethane, chloroform, hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide,1,3-dimethyl-2-imidazolidinone, 1,3-dimethyl-3,4,5,6-tetrahydro-2(1H)-pyrimidinone, acetonitrile, more preferably N,N-dimethylformamide.

The reaction temperature of the above step 7 is in the range from −50° C. to 200° C., preferably from −20° C. to 100° C., though it is not specifically limited so far as the reaction proceeds.

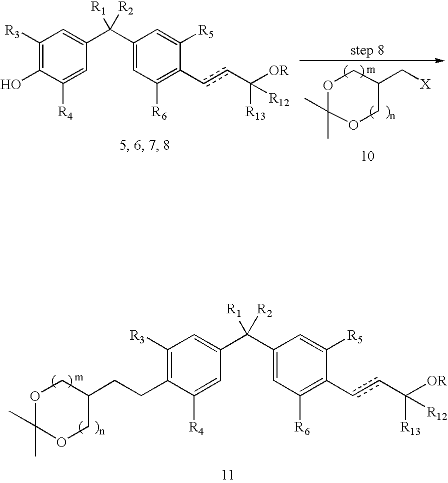

In the above scheme, the compound of general formula (5-8) (R=a hydrogen atom, a methoxymethyl group, a 2-(trimethylsilyl)ethoxymethyl group, a benzyl group, a p-methoxybenzyl group, a trimethylsilyl group, a triethylsilyl group, a t-butyidimethylsilyl group, a t-buthyldiphenylsilyl group or an acetyl group) may be subjected to alkylation with a compound of general formula (10) (m=an integer of 1 to 5, n=an integer of 1 to 5, X=a halogen atom, a methanesulfonyloxy group, a toluenesulfonyloxy group or a trifluoromethanesulfonyloxy group) in the presence of a base to give a compound of general formula (11) (step 8).

Suitable bases for use in the above step 8 include sodium methoxide, sodium ethoxide, sodium tert-butoxide, potassium methoxide, potassium ethoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably sodium hydride or potassium carbonate.

Suitable solvents for use in the above step 8 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably N,N-dimethylformamide.

The reaction temperature of the above step 8 is in the range from −50° C. to 200° C., preferably from 0° C. to 100° C., though it is not specifically limited so far as the reaction proceeds.

In addition, the compound of general formula (5-8) may be subjected to alkylation with a compound of general formula (10) (X=a hydroxyl group) in the presence of triphenylphosphine and dialkyl azocarboxylate to give a compound of general formula (11) in the above scheme (step 8).

In the case using a compound of general formula (10) (X=a hydroxyl group), suitable solvents for use in the above step 8 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (10) (X=a hydroxyl group), the reaction temperature of the above step 8 is in the range from −50° C. to 200° C., preferably from −20° C. to 50° C., though it is not specifically limited so far as the reaction proceeds.

Compound 11 can also be synthesized from compound 4 in order of step 8, step 4, step 5, step 6 and step 7.

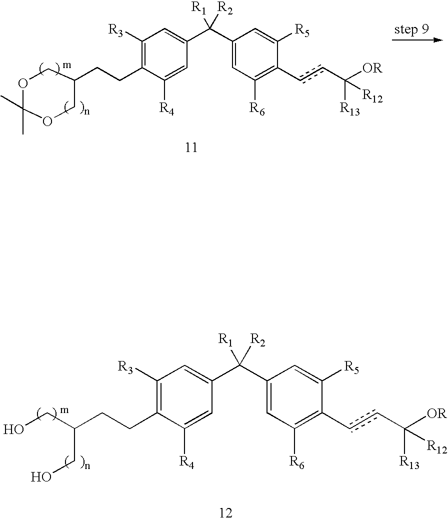

In the above scheme, the compound of general formula (11) may be subjected to deprotection in the presence of an acid to give a compound of general formula (12) (step 9).

Suitable acids for use in the above step 9 include hydrochloric acid, sulfuric acid, acetic acid, phosphoric acid, trifluoroacetic acid, 10-camphorsulfonic acid, p-toluenesulfonic acid, pyridinium p-toluenesuifonic acid, trifluoroborane-diethyl ether complex, carbon tetrabromide, trimethylsilyl bromide, methanesulfonic acid, acidic ion exchange resin.

Suitable solvents for use in the above step 9 include dichloromethane, 1,2-dichloroethane, chloroform, acetone, methanol, ethanol, tetrahydrofuran, 1,2-dimethoxyethane, 1,4-dioxane, diethyl ether, water, the mixtures.

The reaction temperature of the above step 9 is in the range from −10° C. to 150° C., preferably from 0° C. to 100° C., though it is not specifically limited so far as the reaction proceeds.

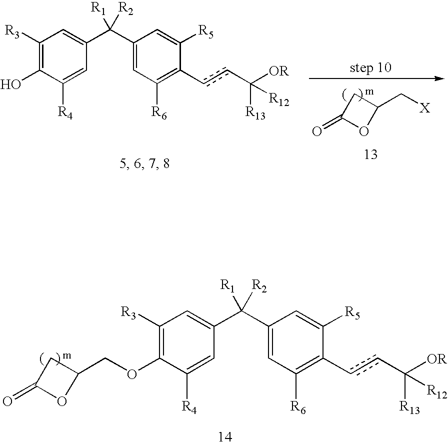

In the above scheme, the compound of general formula (5-8) may be subjected to alkylation with a compound of general formula (13) (m=an integer of 1 to 5, X=a halogen atom, a methanesulfonyloxy group, a toluenesulfonyloxy group or a trifluoromethanesulfonyloxy group) in the presence of a base to give a compound of general formula (14) (step 10).

Suitable bases for use in the above step 10 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 10 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably N,N-dimethylformamide.

The reaction temperature of the above step 10 is in the range from −50° C. to 200° C., preferably from 0° C. to 150° C., though it is not specifically limited so far as the reaction proceeds.

In addition, the compound of general formula (5-8) may be subjected to alkylation with a compound of general formula (13) (X=a hydroxyl group) in the presence of triphenylphosphine and dialkyl azocarboxylate to give a compound of general formula (14) in the above scheme (step 10).

In the case using a compound of general formula (13) (X=a hydroxyl group), suitable solvents for use in the above step 10 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (13) (X=a hydroxyl group), the reaction temperature of the above step 10 is in the range from −50° C. to 200° C., preferably from −20° C. to 50° C., though it is not specifically limited so far as the reaction proceeds.

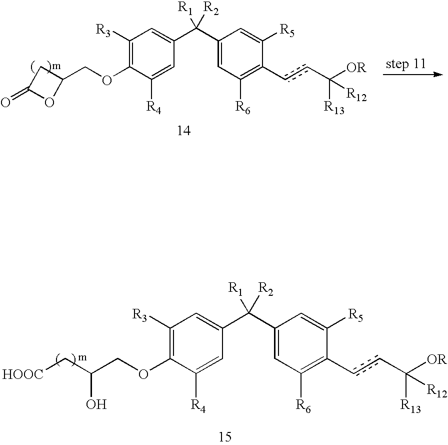

In the above scheme, the compound of general formula (14) may be hydrolyzed in the presence of a base to give a compound of general formula (15) (step 11).

Suitable bases for use in the above step 11 include sodium hydroxide, potassium hydroxide, lithium hydroxide, potassium carbonate, sodium carbonate, sodium hydrogencarbonate, cesium carbonate, more preferably sodium hydroxide or potassium hydroxide.

Suitable solvents for use in the above step 11 include acetone, methanol, ethanol, tetrahydrofuran, 1,4-dioxane, 1,2-dimethoxyethane, water, the mixtures more preferably methanol-water mixture.

The reaction temperature of the above step 11 is in the range from −10° C. to 120° C., preferably from 0° C. to 100° C., though it is not specifically limited so far as the reaction proceeds.

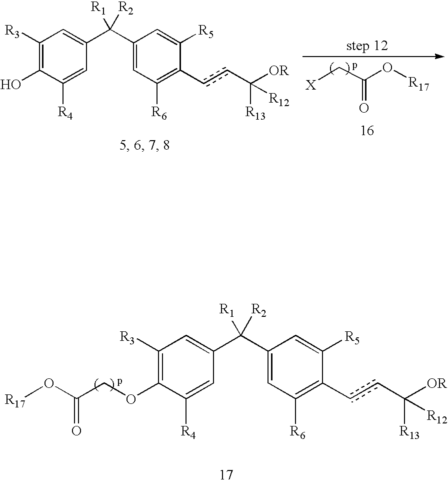

In the above scheme, the compound of general formula (5-8) may be subjected to alkylation with a compound of general formula (16) (p=an integer of 1 to 10, X=a halogen atom, a methanesulfonyloxy group, a toluenesulfonyloxy group or a trifluoromethanesulfonyloxy group) in the presence of a base to give a compound of general formula (17) (step 12).

Suitable bases for use in the above step 12 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 12 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably N,N-dimethylformamide.

The reaction temperature of the above step 12 is in the range from −50° C. to 200° C., preferably from 0° C. to 100° C., though it is not specifically limited so far as the reaction proceeds.

In addition, the compound of general formula (5-8) may be subjected to alkylation with a compound of general formula (16) (X=a hydroxyl group) in the presence of triphenylphosphine and dialkyl azocarboxylate to give a compound of general formula (17) in the above scheme (step 12).

In the case using a compound of general formula (16) (X=a hydroxyl group), suitable solvents for use in the above step 12 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (16) (X=a hydroxyl group), the reaction temperature of the above step 12 is in the range from −50° C. to 200° C., preferably from −20° C. to 50° C., though it is not specifically limited so far as the reaction proceeds.

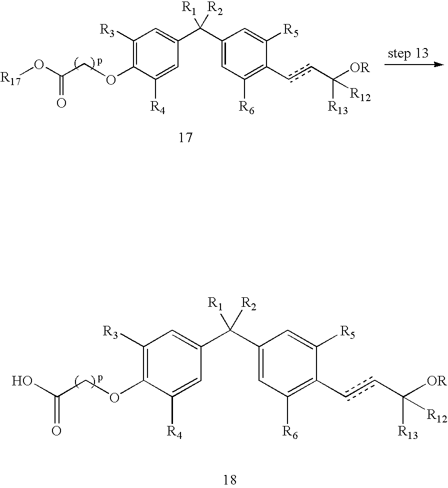

In the above scheme, the compound of general formula (17) may be hydrolyzed in the presence of a base to give a compound of general formula (18) (step 13).

Suitable bases for use in the above step 13 include sodium hydroxide, potassium hydroxide, lithium hydroxide, potassium carbonate, sodium carbonate, sodium hydrogencarbonate, cesium carbonate, more preferably sodium hydroxide or potassium hydroxide.

Suitable solvents for use in the above step 13 include acetone, methanol, ethanol, propanol, isopropanol, tetrahydrofuran, 1,4-dioxane, 1,2-dimethoxyethane, water, the mixtures more preferably methanol-water mixture.

The reaction temperature of the above step 13 is in the range from −10° C. to 120° C., preferably from 0° C. to 100° C., though it is not specifically limited so far-as the reaction proceeds.

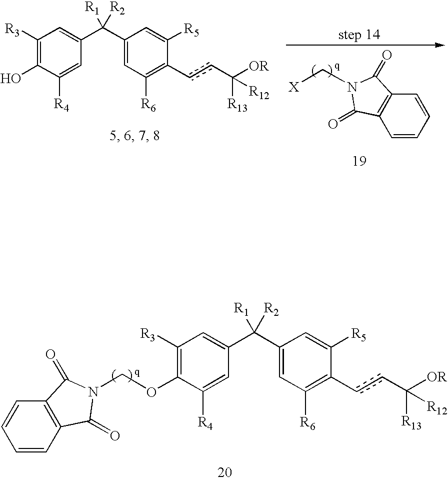

In the above scheme, the compound of general formula (5-8) may be subjected to alkylation with a compound of general formula (19) (q=an integer of 2 to 10, X=a halogen atom, a methanesulfonyloxy group, a toluenesulfonyloxy group or a trifluoromethanesulfonyloxy group) in the presence of a base to give a compound of general formula (20) (step 14).

Suitable bases for use in the above step 14 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 14 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably acetonitrile.

The reaction temperature of the above step 14 is in the range from −50° C. to 200° C., preferably from 0° C. to 100° C., though it is not specifically limited so far as the reaction proceeds.

In addition, the compound of general formula (5-8) may be subjected to alkylation with a compound of general formula (19) (X=a hydroxyl group) in the presence of triphenylphosphine and dialkyl azocarboxylate to give a compound of general formula (20) in the above scheme (step 14).

In the case using a compound of general formula (19) (X=a hydroxyl group), suitable solvents for use in the above step 14 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (19) (X=a hydroxyl group), the reaction temperature of the above step 14 is in the range from −50° C. to 200° C., preferably from −20° C. to 50° C., though it is not specifically limited so far as the reaction proceeds.

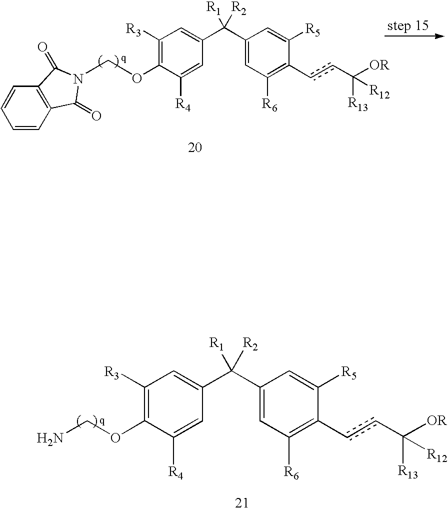

In the above scheme, the step for obtaining a compound of general formula (21) from the compound of general formula (20) may be performed with hydrazine or methylamine (step 15).

Suitable solvents for use in the above step 15 include methanol, ethanol, n-propanol, isopropanol, more preferably methanol. The reaction temperature of the above step 15 is in the range from 0° C. to 200° C., preferably from 0° C. to 100° C., though it is not specifically limited so far as the reaction proceeds.

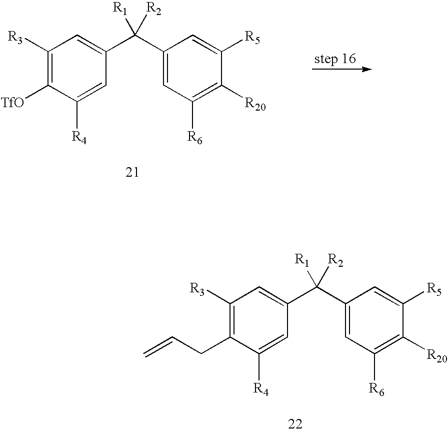

In the above scheme, the compound of general formula (21) (R20=an optionally protected hydroxyl group, an optionally substituted C1-15 alkyl group or an optionally substituted C1-8 alkoxyl group) may be reacted with allyltributyltin in the presence of a palladium catalyst and lithium chloride to give a compound of general formula (22) (step 16).

Suitable palladium catalysts for use in the above step 16 include tetrakis(triphenylphosphine)palladium, Pd(dba)2, Pd2(dba)3-CHCl3, palladium acetate, palladium chloride, [1,1′-bis(diphenylphosphino)-ferrocene]palladium dichloride dichloromethane complex. (dba; dibenzylideneacetone)

A ligand, such as triphenylphosphine, tributhylphosphine, tricyclohexylphosphine, 1,3-bis(diphenylphosphinopropane) or tri-t-buthyl phosphine, may be used in the above step 16 to increase the activity of catalyst or the reaction selectivity.

Suitable solvents for use in the above step 16 include N,N-dimethylformamide, dimethylsulfoxide, N,N-dimethylacetamide, 1,-dimethyl-2-imidazolidinone, tetrahydrofuran, toluene, acetonitrile, more preferably N,N-dimethylformamide.

The reaction temperature of the above step 16 is in the range from 0° C. to 200° C., preferably from 20° C. to 150° C., though it is not specifically limited so far as the reaction proceeds.

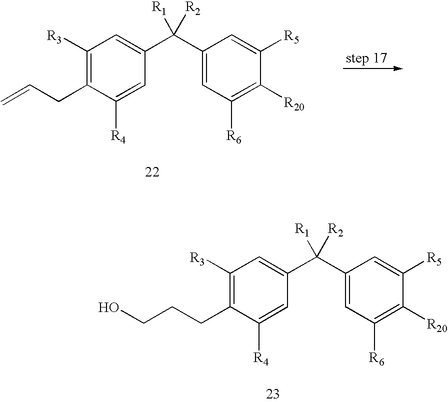

In the above scheme, the compound of general formula (22) (R20=an optionally protected hydroxyl group, an optionally substituted C1-15 alkyl group or an optionally substituted C1-8 alkoxyl group) may be subjected to hydroboration and oxidation to give a compound of general formula (23) (step 17).

Suitable hydroboration reagents for use in the above step 17 include borane-tetrahydrofuran complex, borane-methyl sulfide complex, borane-pyridine complex, borane-trimethylamine complex, or 9-BBN.

Suitable oxidizing agents for use in the above step 17 include hydrogen peroxide-sodium hydroxide, or sodium perborate.

Suitable solvent for use in the above step 17 includes tetrahydrofuran.

The reaction temperature of the above step 17 is in the range from −78° C. to 100° C., preferably from −50° C. to 50° C., though it is not specifically limited so far as the reaction proceeds.

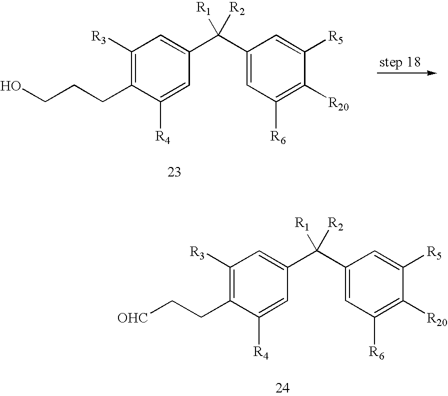

In the above scheme, the compound of general formula (23) (R20=an optionally protected hydroxyl group, an optionally substituted C1-15 alkyl group or an optionally substituted C1-8 alkoxyl group) may be oxidized to give a compound of general formula (24) (step 18).

Suitable oxidizing agents for use in the above step 18 include pyridinium chlorochromate, pyridinium dichromate, manganese dioxide, osmium tetraoxide, ruthenium trichloride, tetrapropylammonium perruthenate, oxaryl chloride/dimethyl sulfoxide/triethylamine, triphosgene/dimethyl sulfoxide, sulfur trioxide pyridine complex/dimethyl sulfoxide, acetone/aluminium triisopropoxide, cyclohexanone/aluminium triisopropoxide, Dess-Martin reagent, more preferably Dess-Martin reagent

Suitable solvents for use in the above step 18 include benzene, toluene, hexane, heptane, dichloromethane, 1,2-dichloroethane, chloroform, tetrahydrofuran, acetone, water, more preferably dichloromethane.

The reaction temperature of the above step 18 is in the range from −78° C. to 50° C., preferably from 0° C. to 50° C., though it is not specifically limited so far as the reaction proceeds.

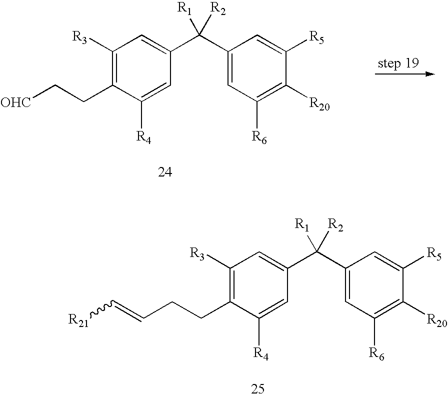

In the above scheme, the compound of general formula (24) (R20=an optionally protected hydroxyl group, an optionally substituted C1-15 alkyl group or an optionally substituted C1-8 alkoxyl group) may be subjected to alkylation with Wittig reagent, such as R21CH2-PPh3G (R21=a hydrogen atom or an optionally substituted C1-15 alkyl group; G=a halogen atom) in the presence of a base to give a compound of general formula (25) (step 19).

Suitable Wittig reagents for use in the above step 19 include methyl triphenylphosphonium bromide, ethyl triphenylphosphonium bromide, n-propyl triphenylphosphonium iodide, isopropyl triphenylphosphonium iodide etc.

Suitable bases for use in the above step 19 include sodium methoxide, sodium ethoxide, sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, more preferably n-butyl lithium.

Suitable solvents for use in the above step 19 include diethyl ether, tetrahydrofuran, benzene, toluene, dimethylsulfoxide, more preferably tetrahydrofuran.

The reaction temperature of the above step 19 is in the range from −78° C. to 120° C., preferably from −78° C. to 50° C., though it is not specifically limited so far as the reaction proceeds.

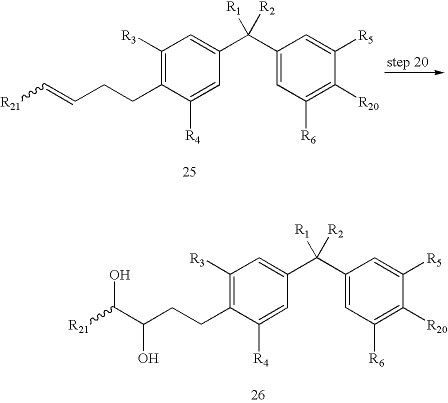

In the above scheme, the compound of general formula (25) (R20=an optionally protected hydroxyl group, an optionally substituted C1-15 alkyl group or an optionally substituted C1-8 alkoxyl group; R21=a hydrogen atom or an optionally substituted C1-15 alkyl group) may be reacted with osmium tetraoxide/4-methylmorpholine N-oxide to give a compound of general formula (26) (step 20).

Suitable solvents for use in the above step 20 include acetone, acetonitrile, t-butanol, water or mixtures, more preferably acetone/t-butanol/water mixture.

The reaction temperature of the above step 20 is in the range from −78° C. to 120° C., preferably from −40° C. to 50° C., though it is not specifically limited so far as the reaction proceeds.

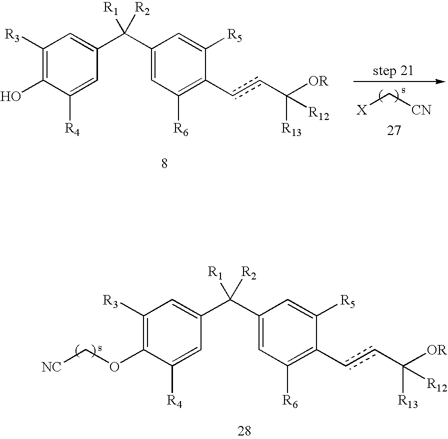

In the above scheme, the compound of general formula (5-8) may be subjected to alkylation with a compound of general formula (27) (s=an integer of 1 to 10, X=a halogen atom, a methanesulfonyloxy group, a toluenesulfonyloxy group or a trifluoromethanesulfonyloxy group) in the presence of a base to give a compound of general formula (28) (step 21).

Suitable bases for use in the above step 21 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 21 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably acetonitrile.

The reaction temperature of the above step 21 is in the range from −50° C. to 200° C., preferably from 0° C. to 100° C., though it is not specifically limited so far as the reaction proceeds.

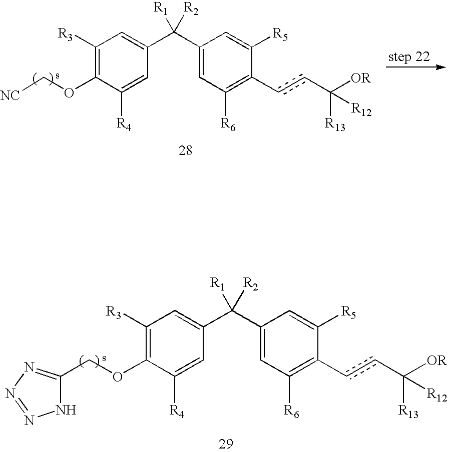

In the above scheme, the step for obtaining a compound of general formula (29) from the compound of general formula (28) may be performed with sodium azide, combination of sodium azide and triethyamine hydrochloride salt, azidotributyltin or combination of trimethylsilylazide and di-n-butyltinoxide (step 22).

Suitable solvents for use in the above step 22 include benzene, toluene, xylene, dyglyme.

The reaction temperature of the above step 22 is in the range from 0° C. to 200° C., preferably from 10° C. to 150° C., though it is not specifically limited so far as the reaction proceeds.

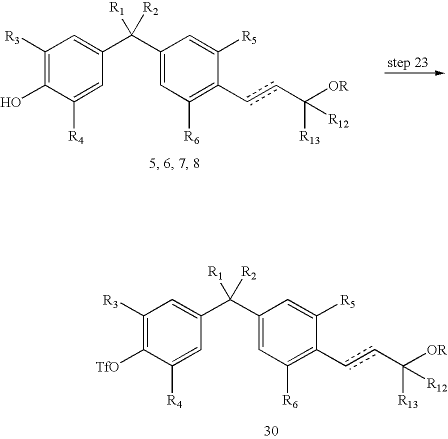

In the above scheme, the compound of general formula (5-8) may be reacted with trifluoromethanesulfonic acid anhydride in the presence of a base to give a compound of general formula (30) (step 23).

Suitable bases for use in the above step 23 include pyridine, 2,6-lutidine, 2,4,6-collidine,N,N-dimethylaminopyridine, imidazole, triethylamine, more preferably pyridine.

Suitable solvents for use in the above step 23 include diethyl ether, tetrahydrofuran, dichloromethane, 1,2-dichloroethane, chloroform, benzene, toluene, more preferably dichloromethane.

The reaction temperature of the above step 23 is in the range from −50° C. to 50° C., preferably from −20° C. to 30° C., though it is not specifically limited so far as the reaction proceeds.

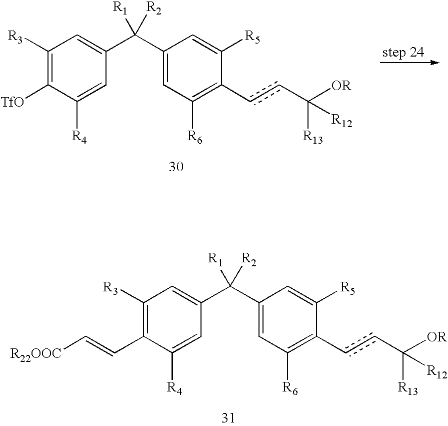

In the above scheme, the compound of general formula (30) may be. reacted with acrylic acid ester in the presence of a palladium catalyst, and triethyamine to give a compound of general formula (31) (R22=a C1-3 alkyl group) (step 24).

Suitable palladium catalysts for use in the above step 24 include tetrakis(triphenylphosphine)palladium, Pd(dba)2, Pd2(dba)3-CHCl3, palladium acetate, palladium chloride, [1,1′-bis(diphenylphosphino)-ferrocene]palladium dichloride dichloromethane complex. (dba; dibenzylideneacetone)

A ligand, such as triphenylphosphine, tributhylphosphine, tricyclohexylphosphine, 1,3-bis(diphenylphosphinopropane) or tri-t-buthyl phosphine, may be used in the above step 24 to increase the catalytic activity and/or the reaction selectivity.

Suitable solvents for use in the above step 24 include N,N-dimethylformamide, dimethylsulfoxide, N,N-dimethylacetamide, 1,3-dimethyl-2-imidazolidinone, tetrahydrofuran, toluene, acetonitrile, more preferably N,N-dimethylformamide or acetonitrile.

The reaction temperature of the above step 24 is in the range from 0° C. to 200° C., preferably from 20° C. to 150° C., though it is not specifically limited so far as the reaction proceeds.

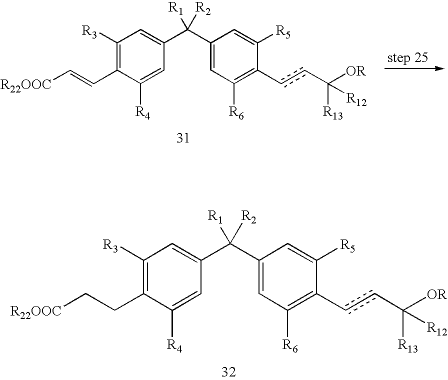

In the above scheme, the compound of general formula (31) (R22=a C1-3 alkyl group) may be reacted with NaBH4 in the presence of NiCl2-6H2O to give a compound of general formula (32) (R22=a C1-3 alkyl group) (step 25).

Suitable solvents for use in the above step 25 include methanol, ethanol, isopropanol, dichloromethane, 1,2-dichloroethane or mixtures, more preferably methanol/dichloromethane mixture.

The reaction temperature of the above step 25 is in the range from −40° C. to 100° C., preferably from −20° C. to 50° C., though it is not specifically limited so far as the reaction proceeds.

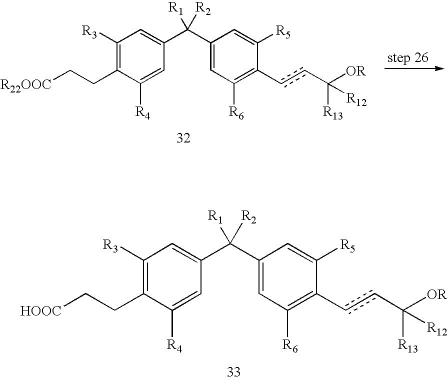

In the above scheme, the compound of general formula (32) (R22=a C1-3 alkyl group) may be hydrolyzed in the presence of a base to give a compound of general formula (33) (step 26).

Suitable bases for use in the above step 26 include sodium hydroxide, potassium hydroxide, lithium hydroxide, potassium carbonate, sodium carbonate, sodium hydrogencarbonate, cesium carbonate, tetrabutylammonium fluoride, more preferably sodium hydroxide, potassium hydroxide or tetrabutylammonium fluoride.

Suitable solvents for use in the above step 26 include acetone, methanol, ethanol, tetrahydrofuran, 1,4-dioxane, 1,2-dimethoxyethane, water, the mixtures more preferably methanol-water mixture or tetrahydrofuran.

The reaction temperature of the above step 26 is in the range from −10° C. to 120° C., preferably from 0° C. to 100° C., though it is not specifically limited so far as the reaction proceeds.

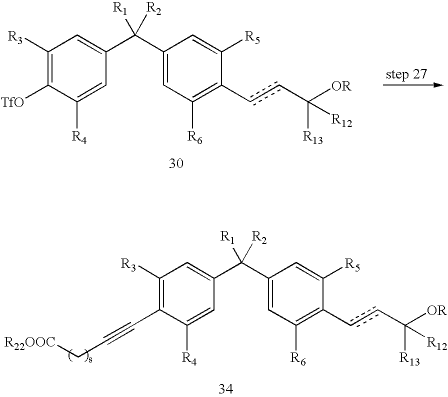

In the above scheme, the compound of general formula (30) may be reacted with acetylene derivative in the presence of a palladium catalyst, copper (I) iodide, and triethyamine to give a compound of general formula (34) (R22=a C1-3 alkyl group) (step 27).

Suitable palladium catalysts for use in the above step 27 include PdCl2(dppf)2-CH2Cl2 complex, tetrakis(triphenylphosphine)palladium, Pd(dba)2, Pd2(dba)3-CHCl3, palladium acetate, palladium chloride. (dba; dibenzylideneacetone, dppf; diphenylphosphino)ferrocene)

A ligand, such as triphenylphosphine, tributhylphosphine, tricyclohexylphosphine, 1,3-bis(diphenylphosphinopropane) or tri-t-buthyl phosphine, may be used in the above step 27 to increase the catalytic activity and/or the reaction selectivity.

Suitable solvents for use in the above step 27 include N,N-dimethylformamide, dimethylsulfoxide, N,N-dimethylacetamide, 1,3-dimethyl-2-imidazolidinone, tetrahydrofuran, toluene, acetonitrile, more preferably N,N-dimethylformamide or acetonitrile.

The reaction temperature of the above step 27 is in the range from 0° C. to 200° C., preferably from 20° C. to 150° C., though it is not specifically limited so far as the reaction proceeds.

The compound of general formula (34) (R22=a C1-3 alkyl group) may be hydrolyzed by the same procedure as described in step 26 to give the corresponding carboxylic acid.

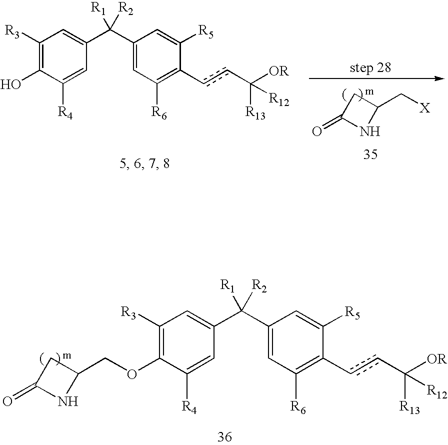

In the above scheme, the compound of general formula (5-8) may be subjected to alkylation with a compound of general formula (35) (m=an integer of 1 to 5, X=a halogen atom, a methanesulfonyloxy group, a toluenesulfonyloxy group or a trifluoromethanesulfonyloxy group) in the presence of a base to give a compound of general formula (36) (step 28).

Suitable bases for use in the above step 28 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 28 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, more preferably N,N-dimethylformamide, acetonitrile.

The reaction temperature of the above step 28 is in the range from −50° C. to 200° C., preferably from 0° C. to 100° C., though it is not specifically limited so far as the reaction proceeds.

In addition, the compound of general formula (5-8) may be subjected to alkylation with a compound of general formula (35) (X=a hydroxyl group) in the presence of triphenylphosphine and dialkyl azocarboxylate to give a compound of general formula (36) in the above scheme (step 28).

In the case using a compound of general formula (35) (X=a hydroxyl group), suitable solvents for use in the above step 28 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (35) (X=a hydroxyl group), the reaction temperature of the above step 28 is in the range from −50° C. to 200° C., preferably from −20° C. to 50° C., though it is not specifically limited so far as the reaction proceeds.

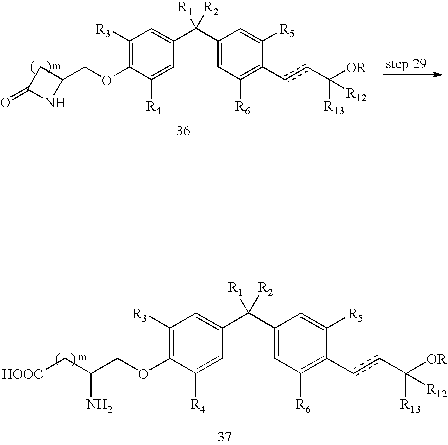

In the above scheme, the compound of general formula (36) may be hydrolyzed in the presence of an acid to give a compound of general formula (37) (step 29).

Suitable acids for use in the above step 29 include hydrochloric acid, hydrobromic acid, sulfuric acid, acetic acid, phosphoric acid, trifluoroacetic acid, 10-camphorsulfonic acid, p-toluenesulfonic acid, methanesulfonic acid, more preferably hydrochloric acid.

Suitable solvents for use in the above step 29 include acetone, methanol, ethanol, tetrahydrofuran, 1,2-dimethoxyethane, 1,4-dioxane, diethyl ether, water, the mixtures.

The reaction temperature of the above step 29 is in the range from 0° C. to 200° C., preferably from 20° C. to 120° C., though it is not specifically limited so far as the reaction proceeds.

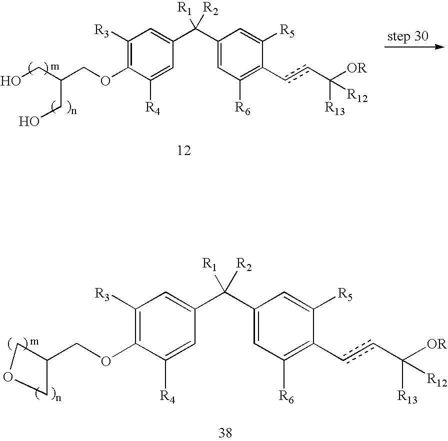

In the above scheme, the compound of general formula (12) may be subjected to cyclization in the presence of triphenylphosphine and dialkyl azocarboxylate to give a compound of general formula (38) (step 30).

Suitable solvents for use in the above step 30 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

The reaction temperature of the above step 30 is in the range from −50° C. to 200° C., preferably from −20° C. to 50° C., though it is not specifically limited so far as the reaction proceeds.

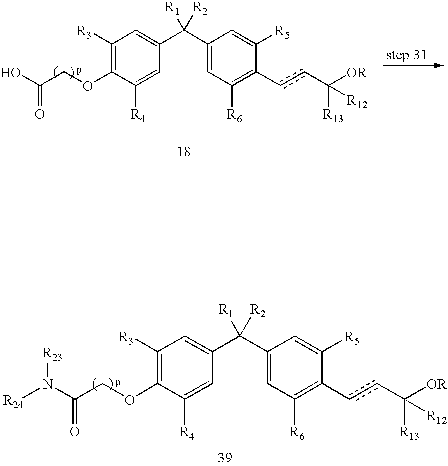

In the above scheme, the compound of general formula (18) may be reacted with amine derivative NHR23(R24) (R23=a hydrogen atom or a C1-3 alkyl group, R24 is represented by following formula) in the presence of coupling reagent used to form amide bonds, after that, may be subjected to deprotection to give a compound of general formula (39) (step 31). R24:

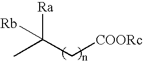

Ra and Rb are each independently selected from the group consisiting of a hydrogen atom, an optionally substituted C1-10 alkyl group, an optionally substituted C3-10 cycloalkyl group, an optionally substituted phenyl group, an optionally substituted benzyl group, an optionally substituted phenethyl group, or Ra and Rb may together form an optionally substituted C3-10 cycloalkyl group.

The substitution group is a hydroxyl group, an amino group, a carboxyl group or a carboxylic amide group with an appropriate protecting group described in Protecting Groups in Organic Synthesis, third edition, John Wiley & Sons, Inc., for example, tert-butyl group, tert-butoxycarbonyl group, trityl group.

Rc is a protecting group selected from a C1-5 alkyl group or polystylene-bound 4-benzyloxy-benzyl group.

In the deprotection step, the cleavage of a protecting group such as tert-butyl group, tert-butoxycarbonyl group, trityl group or polystylene-bound 4-benzyloxy-benzyl group may be carried by trifluoroacetic acid in dichloromethane.

Suitable coupling reagents for use in the above step 31 include 1,3-dicylcohexylcarbodiimide, 1,3-diisopropylcarbodiimide, N-ethyl-N′-(3-dimethylaminopropyl)carbodiimide, benzotriazol-1-yloxy-tris(dimethylamino)phosphonium hexafluorophosphate, diphenylphosphoryl azide.

In the coupling step, a reagent, such as N-hydroxysuccinimide, 1-hydroxybenzotriazole or hydroxy-3,4-dihydro-4-oxo-1,2,3-benzotriazine may be used in the above step 31 to increase the yields.

Suitable solvents for use in the coupling process in step 31 include benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, acetonitrile.

The reaction temperature of the above step 31 is in the range from −50° C. to 200° C., preferably from −20° C. to 50° C., though it is not specifically limited so far as the reaction proceeds.

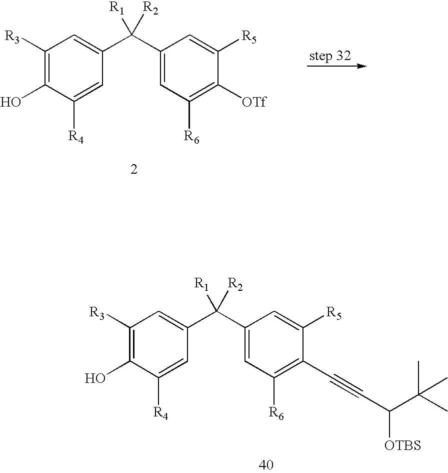

In the above scheme, the compound of general formula (2) may be reacted with tert-butyl-(1-tert-butyl-prop-2-ynyloxy)-dimethyl-silane in the presence of a palladium catalyst, copper (I) iodide, and triethyamine to give a compound of general formula (40) (step 32).

Suitable palladium catalysts for use in the above step 32 include tetrakis(triphenylphosphine)palladium, Pd(dba)2, Pd2(dba)3-CHCl3, palladium acetate, palladium chloride, [1,1′-bis(diphenylphosphino)-ferrocene]palladium dichloride dichloromethane complex. (dba; dibenzylideneacetone)

A ligand, such as triphenylphosphine, tributhylphosphine, tricyclohexylphosphine, 1,3-bis(diphenylphosphinopropane) or tri-t-buthyl phosphine, may be used in the above step 32 to increase the catalytic activity and/or the reaction selectivity.

Suitable solvents for use in the above step 32 include N,N-dimethylformamide, dimethylsulfoxide, N,N-dimethylacetamide, 1,3-dimethyl-2-imidazolidinone, tetrahydrofuran, toluene, acetonitrile, more preferably N,N-dimethylformamide or acetonitrile.

The reaction temperature of the above step 32 is in the range from 0° C. to 200° C., preferably from 20° C. to 150° C., though it is not specifically limited so far as the reaction proceeds.

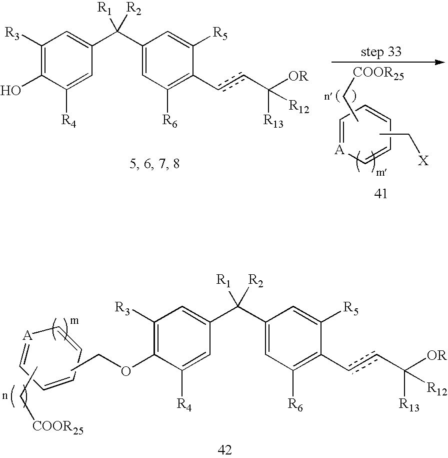

In the above scheme, the compound of general formula (5-8) may be subjected to alkylation with a compound of general formula (41) (m′=0 or 1, n′=0, 1 or 2, A=an oxygen atom, a sulfur atom, a nitrogen atom or a carbon atom, X=a halogen atom, a methanesulfonyloxy group, a toluenesulfonyloxy group or a trifluoromethanesulfonyloxy group, R25=a C1-4 alkyl group) in the presence of a base to give a compound of general formula (42) (step 33).

Suitable bases for use in the above step 33 include sodium tert-butoxide, potassium tert-butoxide, n-butyl lithium, sec-butyl lithium, tert-butyl lithium, lithium diisopropylamide, lithium dicyclohexylamide, lithium bis(trimethylsilyl)amide, potassium bis(trimethylsilyl)amide, sodium hydride, potassium hydride, potassium carbonate, sodium carbonate, sodium hydrogencarbonate, cesium carbonate, more preferably potassium carbonate.

Suitable solvents for use in the above step 33 include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, acetone more preferably N,N-dimethylformamide, acetone.

The reaction temperature of the above step 33 is in the range from −50° C. to 200° C., preferably from 0° C. to 100° C., though it is not specifically limited so far as the reaction proceeds.

In addition, the compound of general formula (5-8) may be subjected to alkylation with a compound of general formula (41) (X=a hydroxyl group) in the presence of triphenylphosphine and dialkyl azocarboxylate to give a compound of general formula (42) in the above scheme (step 33).

In the case using a compound of general formula (41) (X=a hydroxyl group), suitable solvents for use in the above step 33 include hexane, benzene, toluene, diethyl ether, t-butyl methyl ether, tetrahydrofuran, 1,2-dimethoxyethane, 1,4-dioxane, diisopropyl ether, dichloromethane, 1,2-dichloroethane, chloroform, more preferably toluene, tetrahydrofuran.

In the case using a compound of general formula (41) (X=a hydroxyl group), the reaction temperature of the above step 33 is in the range from −50° C. to 200° C., preferably from −20° C. to 50° C., though it is not specifically limited so far as the reaction proceeds.

The compound of general formula (42) (R25=a C1-3 alkyl group) may be hydrolyzed by the same procedure as described in step 26 to give the corresponding carboxylic acid.

The compounds of general formula (8, 11, 12, 14, 15, 17, 18, 20, 21, 28, 29, 30, 31, 32, 33, 34, 36, 37, 38, 39, 40, 42) (R=a methoxymethyl group, a 2-(trimethylsilyl)ethoxymethyl group, a benzyl group, a p-methoxybenzyl group, a trimethylsilyl group, a triethylsilyl group, a t-butyldimethylsilyl group, a t-buthyldiphenylsilyl group or an acetyl group) may be subjected to deprotection in the presence of an acid, a base or a fluorine compound to give the compounds of general formula (8, 11, 12, 14, 15, 17, 18, 20, 21, 28, 29, 30, 31, 32, 33, 34, 36, 37, 38, 39, 40, 42) (R=a hydrogen atom).

Suitable acids for use in the deprotection step of the compounds of general formula (8, 11, 12, 14, 15, 17, 18, 20, 21, 28, 29, 30, 31, 32, 33, 34, 36, 37, 38, 39, 40, 42) (R=a methoxymethyl group, a 2-(trimethylsilyl)ethoxymethyl group, a benzyl group, a p-methoxybenzyl group, a trimethylsilyl group, a triethylsilyl group, a t-butyldimethylsilyl group, a t-buthyldiphenylsilyl group) include hydrochloric acid, hydrobromic acid, sulfuric acid, acetic acid, phosphoric acid, trifluoroacetic acid, formic acid, 10-camphorsulfonic acid, p-toluenesulfonic acid, pyridinium p-toluenesuifonic acid, trifluoroborane-diethyl ether complex, methanesulfonic acid, acidic ion exchange resin.

Suitable bases for use in the deprotection step of the compounds of general formula (8, 11, 12, 14, 15, 17, 18, 20, 21, 28, 29, 30, 31, 32, 33, 34, 36, 37, 38, 39, 40, 42) (R=an acetyl group) include sodium hydroxide, potassium hydroxide, lithium hydroxide, potassium carbonate, sodium carbonate, sodium hydrogencarbonate, cesium carbonate.

Suitable fluorine compounds for use in the deprotection step of the compounds of general formula (8, 11, 12, 14, 15, 17, 18, 20, 21, 28, 29, 30, 31, 32, 33, 34, 36, 37, 38, 39, 40, 42) (R=a 2-(trimethylsilyl)ethoxymethyl group, a trimethylsilyl group, a triethylsilyl group, a t-butyidimethylsilyl group, a t-buthyldiphenylsilyl group or an acetyl group) include tetrabutylammonium fluoride, aqueous hydrogen fluoride, hydrogen fluoride-pyridine, hydrogen fluoride-triethylamine, potassium fluoride, sodium fluoride, calcium fluoride, cesium fluoride.

Suitable solvents for use in the deprotection step of the compounds of general formula (8, 11, 12, 14, 15, 17, 18, 20, 21, 28, 29, 30, 31, 32, 33, 34, 36, 37, 38, 39, 40, 42) (R=a methoxymethyl group, a 2-(trimethylsilyl)ethoxymethyl group, a benzyl group, a p-methoxybenzyl group, a trimethylsilyl group, a triethylsilyl group, a t-butyldimethylsilyl group, a t-buthyldiphenylsilyl group or an acetyl group) include hexane, benzene, toluene, diethyl ether, t-butyl methyl ether, tetrahydrofuran, 1,2-dimethoxyethane, 1,4-dioxane, diisopropyl ether, N,N-dimethylformamide, dimethylsulfoxide, N,N-dimrethylacetamide, 1,3-dimethyl-2-imidazolidinone, 1,3-dimethyl-3,4,5,6-tetrahydro-2(1H)-pyrimidinone, methanol, ethanol, isopropanol, acetonitrile, acetone, water, the mixtures.

The reaction temperature in the deprotection step of the compounds of general formula (8, 11, 12, 14, 15, 17, 18, 20, 21, 28, 29, 30, 31, 32, 33, 34, 36, 37, 38, 39, 40, 42) (R=a methoxymethyl group, a 2-(trimethylsilyl)ethoxymethyl group, a benzyl group, a p-methoxybenzyl group, a trimethylsilyl group, a triethylsilyl group, a t-butyldimethylsilyl group, a t-buthyldiphenylsilyl group or an acetyl group) is in the range from −50° C. to 200° C., preferably from −20° C. to 120° C., though it is not specifically limited so far as the reaction proceeds.

### EXAMPLES

To further illustrate this invention, the following examples are included. The examples should not, of course, be construed as specifically limiting the invention. Variations of these examples are within the purview of one skilled in the art and considered to fall within the scope of the invention as described and claimed herein. The reader will recognize that the skilled artisan, in possession of the present disclosure and skilled in the art, is able to prepare and use the invention without exhaustive examples.

Materials used herein identified with trademarks are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

Reactions were performed under nitrogen or argon atmosphere at room temperature in stirring condition, otherwise indicated.

Chromatographs were performed with nitrogen pressure using silica gel from Kanto Chemicals (silica gel 60N (Spherical neutral) 40-50 micro m) or Merck (silica gel 60 (230-400 mesh ASTM) 40-50 micro m).

Nuclear magnetic resonanse (NMR) analyses were performed on AXR 300 (Bruker), a EX-270 sectrometer (JEOL), or Gemini2000/300 (Varian) in CDCl₃ using tetramethylsilane as a internal standard, otherwise indicated.

Mass spectrum (MS) analyses were performed on ZQ2000 (Waters), LCQ.Classic (Thermo), or Q-micro, Triple Quadrupole Mass Spectrometer (MICROMASS) in APCI/ESI (atmospheric chemical pressure ionization/electron spray ionization) negative or positive mode.

The following abbreviations have the indicated meanings:

| | |
|---|---|
| (Boc)₂O | di-tert-butyl dicarbonate |
| 1,25(OH)₂D₃ | 1a,25-dihydroxyvitamin D3 |
| 1M | 1 mol/l |
| 1N | 1 normal |
| Abu | gamma-aminobutylic acid |
| Ac₂O | acetic anhydride |
| AcOEt | ethyl acetate |
| aq. | aqueous |
| BF₃-OEt₂ | boron trifluoride diethyl etherate |
| BnOH | benzyl alcohol |
| Boc | tert-butoxycarbonyl |
| CH₃CN | acetonitrile |
| Cha | beta-cyclohexylalanine |
| Chg | cyclohexylglycine |
| chloroform-d | CDCl3 |
| conc | concentrated |
| CSA | (1S)-(+)-10-camphorsulfonic acid |
| DCC | dicyclohexylcarbodiimide |
| DEAD | Diethyl azodicarboxylate |
| Dess-Martin reagent | 1,1,1-tris(acetyloxy)-1,1-dihydro-1,2- |
| | benziodoxol-3-(1H)-one |
| DHP | dihydropyran |
| DMAP | 4-(dimethylamino)pyridine |
| DMF | N,N-Dimethylformamide |
| DMSO | dimethylsulfoxide |
| dppp | diphenylphosphinopropane |
| EDC | 1-ethyl-3-(3-dimethylaminopropyl)- |
| | cabodiimide |
| Et | ethyl |
| Et₂O | diethylether |
| Et₃N | Triethylamine |
| Et₃SiCl | triethylsilyl chloride |
| EtLi | ethyl lithium |
| EtMgBr | ethyl magnesium bromide |
| EtOAc | ethyl acetate |
| EtOH | ethanol |
| Fmoc | 9-fluorenylmethoxycarbonyl |
| Hex | n-hexane |
| HMPA | hexamethylphosphoramide |
| HOBT | 1-hydroxybenzotriazole |
| HPLC | high performance liquid chromatography |
| i-Pr₂EtN | diisopropylethylamine |
| i-PrOH, iPrOH | isopropanol |
| LAH | lithium aluminum hydride |
| LDA | lithium diisopropylamide |
| mCPBA | m-chloroperbenzoic acid |
| Me | methyl |
| MeCN | acetonitrile |
| MeOH | methanol |
| methanol-d4 | CD₃OD |
| MOM | methoxymethyl |
| MS4A | molecular sieves 4A |
| MsCl | methanesulfonyl chloride |
| NaOMe | sodium methoxide |
| n-BuLi, nBuLi, BuLi | n-butyl lithium |
| n-Hex | n-hexane |
| Nle | norleucine |
| NMO | N-methylmorpholine-N-oxide |
| Nva | norvaline |
| ODS | octadecylsilyl |
| Orn | ornitine |
| PCC | pyridinium chlorochromate |
| Pd(OAc)₂ | Palladium acetate |
| Pd(OH)₂/C | palladium hydroxide on carbon |
| Pd(PPh₃)₄ | tetrakis(triphenylphosphine)palladium |
| Pd-C | palladium on activated carbon |
| PdCl₂(dppf)₂ | [1,1′- |
| | bis(diphenylphosphino)ferrocene]palladium |
| | dichloride |
| PdCl₂(PPh₃)₂ | dichlorobis(triphenylphosphine)palladium |
| Phg | phenylglycine |
| Piv | pivaloyl |
| PPh₃ | triphenylphosphine |
| PPTs | pyridinium p-toluenesulfonate |
| PTH | parathyroid hormone |
| pTsOH | p-toluenesulfonic acid |
| quant. | quantitative yield |
| rt | room temperature |
| sat. | saturated |
| TBAF | tetrabutylammonium fluoride |
| TBDMS | tert-butyldimethylsilyl |
| TBS | tert-butyldimethylsilyl |
| TBSCl | tert-butyldimethylsilyl chloride |
| TBSOTf | tert-butyldimethysilyl |
| | trifluoromethanesulfonate |
| tBu | tert-butyl |
| t-BuLi | tert-butyl lithium |
| t-BuNH₂ | tert-butylamine |
| tBuOH | tert-butylalcohol |
| t-BuOK | potassium tert-butoxide |
| t-BuSH, tBuSH | 2-methyl-2-propanethiol |
| TEMPO | 2,2,6,6-tetramethyl-1-piperidinyloxy, free |
| | radical |
| TES | triethylsillyl |
| Tf | trifluoromethanesulfonyl |
| Tf₂O | Trifluoromethanesulfonic anhydride |
| TFA | trifluoroacetic acid |
| THF | Tetrahydrofuran |
| THP | tetrahydropyranyl |
| TLC | thin-layer chromatography |
| TMS | trimethylsilyl |
| TMS-acetylene | trimethylsilylacetylene |
| TMSBr | trimethylsilyl bromide |
| Trt | trityl |
| Ts | p-toluenesulfonyl |
| v/v | volume/volume |
| Wang Resin | p-hydroxymethyl-phenoxy-methylated |
| | polystylene |
| WSCI | 1-ethyl-3-(3-dimethylaminopropyl)- |
| | cabodiimide |

### Example 1

### Preparation of (S)-5-(4-{1-ethyl-1-[4-((E)-3-ethyl-3-hydroxy-pent-1-enyl)-3-methyl-phenyl]-propyl}-2-methyl-phenoxy)-4-hydroxy-pentanoic acid

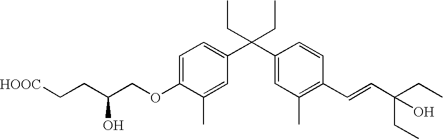

### (1) Preparation of trifluoro-methanesulfonic acid 4-[1-ethyl-1-(4-hydroxy-3-methyl-phenyl-propyl]-2-methyl-phenyl ester

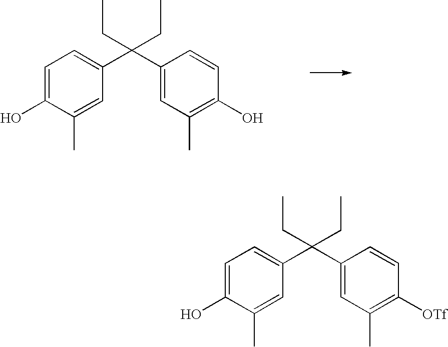

To a solution of 3,3-bis[4-hydroxy-3-methylphenyl]pentane (9.0 g, 31.6 mmol) in dichloromethane (300 ml), pyridine (3 ml, 37.2 mmol) and trifluoromethanesulufonic anhydride (5.7 ml, 34.7 mmol) were added at 0° C. and stirred at 0° C. for 1 h. To the mixture, ethylacetate was added and the organic layer was washed with saturated NaHCO₃ solution and brine, dried over MgSO₄, concentrated in vacuo, and chromatographed (ethylacetate/hexane=1/10 to ethyl acetate only) to give the title compound (4.9 g, 37%).

¹H-NMR: 0.62 (t, 6H), 2.03 (q, 4H), 2.20 (s, 3H), 2.38 (s, 3H), 4.67 (s, 1H), 6.68 (d, 1H), 6.80-6.88 (m, 2H), 7.02-7.11 (m, 3H); MS (ESI+):417 ([M+H]⁺).

### (2) Preparation of 4-[1-ethyl-1-(3-methyl-4-trimethylsilanylethynyl-phenyl)-propyl]-2-methyl-phenol

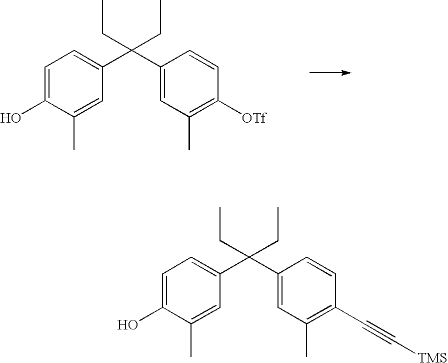

To a solution of trifluoromethanesulfonic acid 4-[1-ethyl-1-(4-hydroxy-3-methyl-phenyl)-propyl]-2-methyl-phenyl ester (compound prepared in Example 1-(1)) (25 g, 60.03 mmol) in acetonitrile (300 ml), triethylamine (25.1 ml, 180.09 mmol), ethynyltrimethylsilane (25.5 ml, 180.09 mmol), Cul (1.143 g, 6.00 mmol), and Pd(PPh₃)₄ (6.93 g, 6.00 mmol) were added. The mixture was stirred at 110° C. for 18 h and concentrated in vacuo. To the residue, ethylacetate and H₂O added and the pH was adjusted at 7 by addition of 1 N HCl. The organic layer was dried over MgSO₄, concentrated in vacuo, and chromatographed (ethylacetate/hexane=1/15) to give the title compound (16 g, 74%).

¹H-NMR: 0.25 (s, 9H), 0.60 (t, 6H), 2.04 (q, 4H), 2.18 (s, 3H), 2.38 (s, 3H), 4.58 (s, 1H), 6.63 (d, 1H), 6.83-6.88 (m, 2H), 6.92 (d, 1H), 7.00 (s, 1H), 7.30 (d, 1H).

### (3) Preparation of 4-[1-ethyl-1-(4-ethynyl-3-methyl-phenyl)-propyl]-2-methyl-phenol

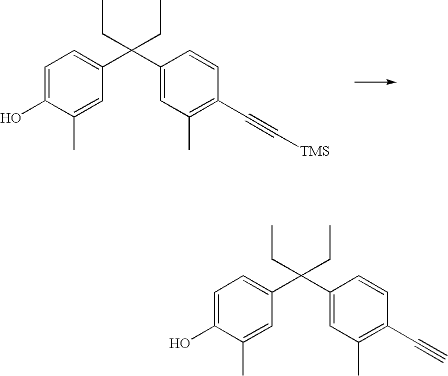

To a solution of 4-[1-ethyl-1-(3-methyl-4-trimethylsilanylethynyl-phenyl)-propyl]-2-methyl-phenol (compound prepared in Example 1-(2)) (19.03 g, 52.19 mmol) in THF (520 ml), 1 M terabutylanmonium fluoride in THF (78.3 ml, 78.3 mmol) was added. The mixture was stirred for 0.5 h, concentrated in vacuo, extracted with ethylacetate,.washed with brine, dried over MgSO₄, concentrated in vacuo, and chromatographed (ethylacetate/hexane=1/10) to give the title compound (15.6 g, 94%).

¹H-NMR: 0.60 (t, 6H), 2.04 (q, 4H), 2.20 (s, 3H), 2.41 (s, 3H), 3.23 (s, 1H), 4.51 (s, 1H), 6.64 (d, 1H), 6.82-6.86 (m, 2H), 6.90-6.95 (m, 1H), 7.00 (s, 1H), 7.29 (d, 1H); MS (ESI−) : 291 ([M−H]⁻).

### (4) Preparation of 4-{1-ethyl-1-[4-(3-ethyl-3-hydroxy-pent-1-ynyl)-3-methyl-phenyl]-propyl}-2-methyl-phenol

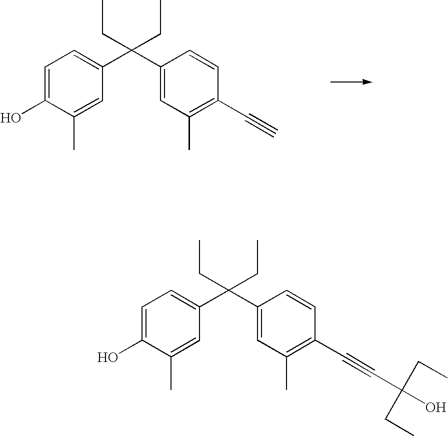

To a solution of 4-[1-ethyl-1-(4-ethynyl-3-methyl-phenyl)-propyl]-2-methyl-phenol (compound prepared in Example 1-(3)) (4.28 g, 14.69 mmol) in THF (92 ml), 2.5 M n-butyl lithium in hexane (14.7 ml, 36.73 mmol) was added at −78° C. To the mixture, pentan-3-one (4.85 ml, 44.07 mmol) was added at −78° C., stirred at −78° C. for 3 h, and concentrated in vacuo. To the residue, ethylacetate and H₂O were added and the pH was adjusted at 6 by addition of 1 N HCl. Extracted with ethylacetate, dried over Na₂SO₄, concentrated in vacuo, and chromatographed (ethylacetate/hexane=1/6) to give the title compound (3.66 g, 62%).

¹H-NMR: 0.61 (t, 6H), 1.11 (t, 6H), 1.72-1.78 (m, 4H), 2.02 (q, 4H), 2.19 (s, 3H), 2.38 (s, 3H), 4.50 (s, 1H), 6.65 (d, 1H), 6.82-6.86 (m, 2H), 6.90-6.95 (m, 1H), 7.00 (s, 1H), 7.29 (d, 1H); MS (ESI−): 377 ([M−H]⁻).

### (5) Preparation of 4-{1-ethyl-1-[4-(3-ethyl-3-hydroxy-(1E)-pent-1-enyl)-3-methyl-phenyl]-propyl}-2-methyl-phenol

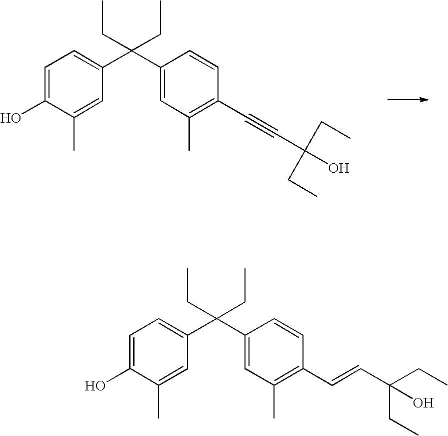

To a solution of 4-{1-ethyl-1-[4-(3-ethyl-3-hydroxy-pent-1-ynyl)-3-methyl-phenyl]propyl}-2-methyl-phenol (compound prepared in Example 1-(4)) (800 mg, 2.11 mmol) in THF (8 ml), 1 M LiAlH₄ in THF (5.28 ml, 5.28 mmol) was added. The mixture was refluxed for 18 h and H₂O was added after cooling. Filtrated, extracted with ethylacetate, washed with brine, dried over MgSO₄, concentrated in vacuo, and chromatographed (ethylacetate/hexane=1/7) to give the title compound (500 mg, 62%).

¹H-NMR: 0.60 (t, 6H), 0.93 (t, 6H), 1.64 (q, 4H), 2.04 (q, 4H), 2.20 (s, 3H), 2.31 (s, 3H), 4.59 (s, 1H), 6.02 (d, 1H), 6.66 (d, 1H), 6.75 (d, 1H), 6.83-6.92 (m, 4H), 7.28 (d, 1H) MS (ESI−): 379 ([M−H]⁻).

### (6) Preparation of (S)-5-(4-{1-ethyl-1-[4-((E)-3-ethyl-3-hydroxy-pent-1-enyl)-3-methyl-phenyl]-propyl}-2-methyl-phenoxymethyl)-dihydro-furan-2-one

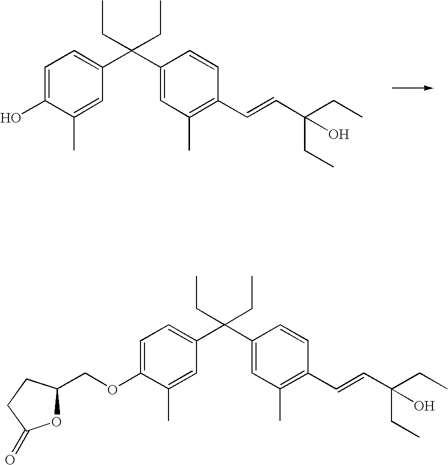

To a solution of 4-{1-ethyl-1-[4-(3-ethyl-3-hydroxy-(1E)-pent-1-enyl)-3-methyl-phenyl]-propyl}-2-methyl-phenol (compound prepared in Example 1-(5)) (150 mg, 0.394 mmol) in dimethylformamide (4 ml), K₂CO₃ (136 mg, 0.985 mmol) was added and stirred for 0.5 h. To the mixture, toluene-4-sulfonic acid (S)-5-oxo-tetrahydro-furan-2-ylmethyl ester (160 mg, 0.591 mmol) was added and stirred at 100° C. for 15 h. After cooling to room temperature, saturated NH₄Cl solution was added. Extracted with ethylacetate, washed with brine, dried over MgSO₄, concentrated in vacuo, and chromatographed (ethylacetate/hexane=1/5 to 1/3) to give the title compound (170 mg, 90%).

¹H-NMR: 0.62 (t, 6H), 0.92 (t, 6H), 1.65 (q, 4H), 2.04 (q, 4H), 2.17 (s, 3H), 2.32 (s, 3H), 2.34-2.84 (m, 4H), 4.05-4.20 (m, 2H), 4.85-4.92 (m, 1H), 6.02 (d, 1H), 6.67 (d, 1H), 6.75 (d, 1H), 6.90-6.97 (m, 4H), 7.29 (d, 1H); MS (ESI+): 501 ([M+Na]⁺).

### (7) Preparation of (S)-5-(4-{1-ethyl-1-[4-((E)-3-ethyl-3-hydroxy-pent-1-enyl)-3-methyl-phenyl]-propyl}-2-methyl-phenoxy)-4-hydroxy-pentanoic acid

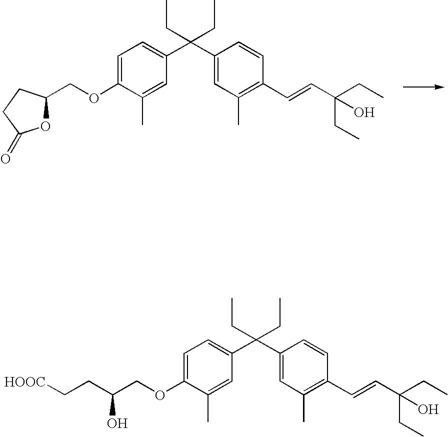

To a solution of (S)-5-(4-{1-ethyl-1-[4-((E)-3-ethyl-3-hydroxy-pent-1-enyl)-3-methyl-phenyl]-propyl}-2-methyl-phenoxymethyl)-dihydro-furan-2-one (compound prepared in Example 1-(6)) (170 mg, 0.355 mmol) in methanol (7 ml), 1 N KOH solution (0.7 ml) was added. The mixture was stirred for 2 h, concentrated in vacuo, extracted with ethylacetate, washed with brine, dried over MgSO₄, concentrated in vacuo, and chromatographed (dichloromethane/methanol=20/1) to give the title compound (150 mg, 85%).

¹H-NMR (measured in CD₃OD as potassium salt): 0.63 (t, 6H), 0.97 (t, 6H), 1.68 (q, 4H), 1.84-1.94 (m, 1H), 1.97-2.04 (m, 1H), 2.10 (q, 4H), 2.21 (s, 3H), 2.32 (s, 3H), 2.42 (t, 2H), 3.93-4.03 (m, 3H), 6.04 (d, 1H), 6.78 (d, 1H), 6.81 (s, 1H), 6.91 (s, 1H), 6.95-7.00 (m, 3H), 7.31 (d, 1H); MS (ESI−): 495 ([M−H]⁻).

### Example 2

### Preparation of (S)-5-(4-{1-ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid

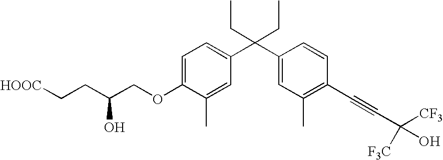

### (1) Preparation of 4-{1-ethyl-1-[3-methyl4-(4,4,4-trifluoro-3-hydroxy-3-trifluoromethyl-but-1-ynyl)-phenyl]-propyl}-2-methyl-phenol

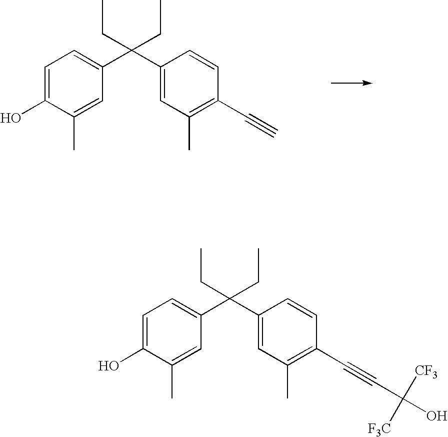

To a solution of 4-[1-ethyl-1-(4-ethynyl-3-methyl-phenyl)-propyl]-2-methyl-phenol (compound prepared in Example 1-(3)) (4.4 g, 15.1 mmol) in THF (108 ml), 2.5 M n-butyl lithium in hexane (12 ml, 30.2 mmol) was added at −78° C. and stirred at −78° C. for 1 h. To the mixture, hexafluoroacetone was passed by bubbling at −78° C. and stirred at −78° C. for 2 h. To the reaction mixture, diethylether and H₂O were added, and the pH was adjusted at 7 by addition of 1 N HCl. Extracted with diethyl ether, dried over MgSO₄, concentrated in vacuo, chromatographed two times (ethyl acetate/hexane=1/10 and 1/5), and purified by HPLC (column:Merck Lobar column 40-63 micrometer 400×40 mm (ODS), MeOH/H₂O=2/1, 3/1, and 4/1, isocratic) to give the title compound (2.0 g, 29%).

¹H-NMR: 0.61 (t, 6H), 2.05 (q, 4H), 2.20 (s, 3H), 2.39 (s, 3H), 3.50 (s,1H), 4.58 (s, 1H), 6.66 (d, 1H), 6.80-6.83 (m, 2H), 6.99 (d, 1H), 7.07 (s, 1H), 7.34 (d, 1H).

### (2) Preparation of 4-{1-ethyl-1-[3-methyl-4-(4,4,4-trifluoro-3-methoxymethoxy-3-trifluoromethyl-but-1-ynyl)-phenyl]-propyl}-2-methyl-phenol

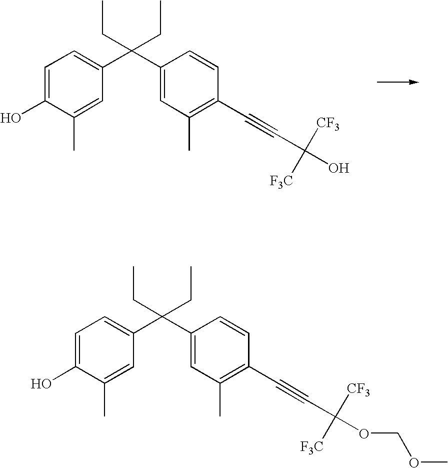

To a solution of 4-{1-ethyl-1-[3-methyl-4-(4,4,4-trifluoro-3-hydroxy-3-trifluoromethyl-but-1-ynyl)-phenyl]-propyl}-2-methyl-phenol (compound prepared in Example 2-(1)) (356 mg, 0.777 mmol) in dimethylformamide (5 ml), K₂CO₃ (268 mg, 1.94 mmol) was added and stirred for 20 min. To the mixture, chloromethoxymethane (71 μl, 0.935 mmol) was added and stirred for 1 h. To the reaction mixture, saturated NH₄Cl (20 ml) was added. Extracted with ethylacetate and dried over MgSO₄, concentrated in vacuo, and chromatographed (ethylacetate/hexane=1/6) to give the title compound (349 mg, 89 %).

¹H-NMR: 0.60 (t, 6H), 2.05 (q, 4H), 2.20 (s, 3H), 2.40 (s, 3H), 3.49 (s, 3H), 4.59 (s, 1H), 5.17 (s, 2H), 6.65 (d, 1H), 6.80-6.83 (m, 2H), 6.99 (d, 1H), 7.07 (s, 1H), 7.38 (d, 1H).

### (3) Preparation of (S)-5-(4-{1-ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one

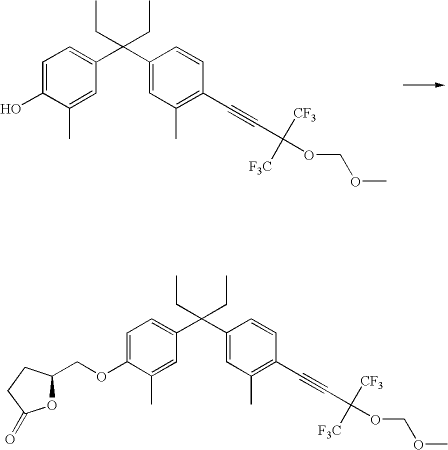

To a solution of 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) (300 mg, 0.597 mmol) in dimethylformamide (3 ml), 60% NaH (29 mg, 0.725 mmol) was added and stirred for 0.5 h. To the mixture, toluene-4-sulfonic acid (S)-5-oxo-tetrahydro-furan-2-ylmethyl ester (194 mg, 0.719 mmol) was added and stirred at 110-120° C. for 16 h. To the reaction mixture, saturated NH₄Cl solution was added. Extracted with ethylacetate, washed with brine, dried over MgSO₄, concentrated in vacuo, and chromatographed (ethylacetate/hexane=1/3) to give the title compound (158 mg, 44%)

¹H-NMR: 0.60 (t, 6H), 2.05 (q, 4H), 2.17 (s, 3H), 2.42 (s, 3H), 2.25-2.80 (m, 4H), 3.49 (s, 3H), 4.04-4.17 (m, 2H), 4.86-4.92 (m, 1H), 5.17 (s, 2H), 6.67 (d, 1H), 6.83 (s, 1H), 6.92 (d, 1H), 6.98 (d, 1H), 7.02 (s, 1H), 7.38 (d, 1H).

### (4) Preparation of (S)-5-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one

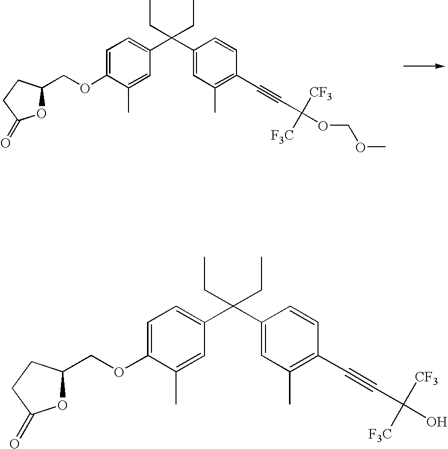

To a solution of (S)-5-(4-{1-ethyl-1-[3-methyl4-(4,4,4-trifluoro-3-methoxymethoxy-3-trifluoromethyl-but-1-ynyl)-phenyl]-propyl}-2-methyl-phenoxymethyl)-dihydro-furan-2-one (compound prepared in Example 2-(3)) (338 mg, 0.562 mmol) in dichloromethane (10 ml), trifluoroacetic acid (1 ml) was added and stirred for 16 h. Concentrated in vacuo, and chromatographed (ethylacetate/hexane=1/3) to give the title compound (153 mg, 49%).

¹H-NMR: 0.60 (t, 6H), 2.05 (q, 4H), 2.14 (s, 3H), 2.39 (s, 3H), 2.25-2.81 (m, 4H), 4.04-4.17 (m, 2H), 4.86-4.91 (m, 1H), 6.63 (d, 1H), 6.83 (s, 1H), 6.90 (dd, 1H), 6.96 (dd, 1H), 7.02 (s, 1H), 7.38 (d, 1H); MS (ESI−): 555 ([M−H]⁻).

### (5) Preparation of (S)-5-(4-{1-ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid

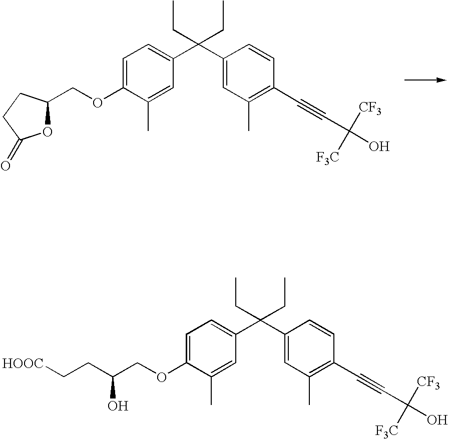

To a solution of (S)-5-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one (compound prepared in Example 2-(4)) (70.2 mg, 0.126 mmol) in ethanol (3 ml), 1 N KOH solution (0.378 ml) was added and stirred for 3 h. Concentrated in vacuo and extracted with diethylether. The pH was adjusted at 9 by addition of saturated KHSO₄. The organic layer was filtrated through celite and concentrated in vacuo to give the title compound (36.7 mg, 52%).

¹H-NMR (measured in CD₃OD as potassium salt): 0.60 (t, 6H), 1.77-1.88 (m, 1H), 1.93-2.03 (m, 1H), 2.09 (q, 4H), 2.15 (s, 3H), 2.38 (s, 3H), 2.38-2.44 (m, 2H), 3.88-3.92 (m, 2H), 3.93-4.00 (m, 1H), 6.78 (d, 1H), 6.84 (s, 1H), 6.94 (dd, 1H), 7.03 (d, 1H), 7.09 (s, 1H), 7.34 (d, 1H); MS (ESI−): 573 ([M−H]⁻).

### Example 3

### Preparation of 4-{1-ethyl-1-[4-(3-hydroxy-4,4-dimethyl-pent-1-ynyl)-3-methyl-phenyl]-propyl}-2-methyl-phenol

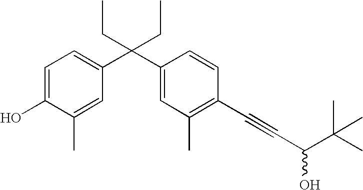

### Preparation of 4-{1-ethyl-1-[4-(3-hydroxy-4,4-dimethyl-pent-1-ynyl)-3-methyl-phenyl]-propyl}-2-methyl-phenol

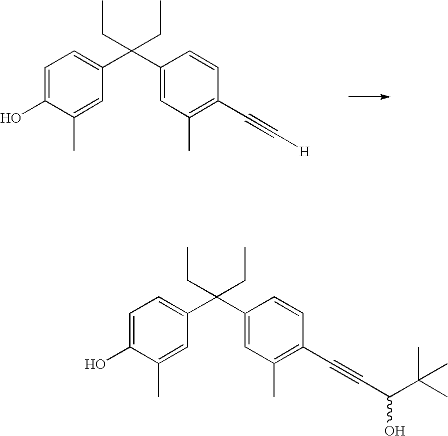

To a solution of 4-[1-ethyl-1-(4-ethynyl-3-methyl-phenyl)-propyl]-2-methyl-phenol (compound prepared in Example 1-(3)) (12.22 g, 41.8 mmol) in THF (300 ml) was added 2.44 M n-butyl lithium in hexane (42.8 ml, 104.5 mmol) at 0 degrees C. The reaction mixture was stirred for 30 min at 0 degrees C. Trimethylacetaldehyde (13.79 ml, 125.4 mmol) was added, stirred at 0 degrees C. for 30 min. To the reaction mixture, saturated aqueous NH₄Cl was added and extracted with ethyl acetate, washed with brine, dried over MgSO₄, concentrated in vacuo, and chromatographed on silica gel (ethyl acetate/hexane=10/90 to 25/75) to give the title compound (13.08 g, 83%).

¹H-NMR(CDCl₃): 0.59 (t, 6H), 1.07 (s, 9H), 1.86 (d, 1H), 2.03 (q, 4H), 2.19 (s, 3H), 2.38 (s, 3H), 4.26 (d, 1H), 4.59 (s, 1H), 6.65 (d, 1H), 6.82-6.85 (m, 2H), 6.92-6.95 (m,1H), 7.00 (s, 1H), 7.28 (d, 1H).

### Example 4

### Preparation of 4-{1-ethyl-1-[4-(3-hydroxy-3-propyl-hex-1-ynyl)-3-methyl-phenyl]-propyl}-2-methyl-phenol

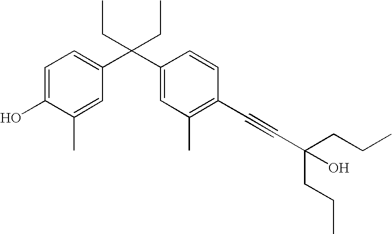

### Preparation of 4-{1-ethyl-1-[4-(3-hydroxy-3-propyl-hex-1-ynyl)-3-methyl-phenyl]-propyl}-2-methyl-phenol

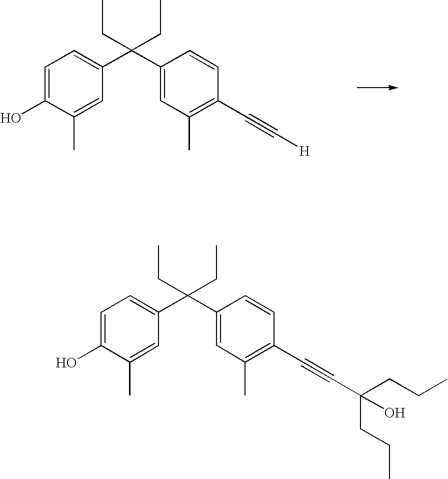

Using the same procedure as described for the preparation of Example 3, the title compound was prepared from 4-[1-ethyl-1-(4-ethynyl-3-methyl-phenyl)-propyl]-2-methyl-phenol (compound prepared in Example 1-(3)).

¹H-NMR(CDCl₃): 0.60 (t, 6H), 0.92(t, 3H), 1.30-1.61(m, 8H),2.05(q, 4H), 2.19(s,3H), 2.30(s,3H), 4.70(s, 1H), 6.65(d, 1H), 6.83(m, 2H), 7.00(d,1 H), 7.18(d, 1H), 7.28(d, 1H); MS (ESI−) :405 ([M−H]⁻).

### Example 5

### Preparation of 4-{1-ethyl-1-[4-(3-hydroxy-3-methyl-but-1-ynyl)-3-methyl-phenyl]-propyl}-2-methyl-phenol

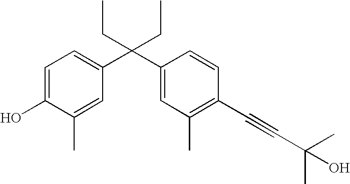

### Preparation of 4-{1-ethyl-1-[4-(3-hydroxy-3-methyl-but-1-ynyl)-3-methyl-phenyl]-propyl}-2-methyl-phenol

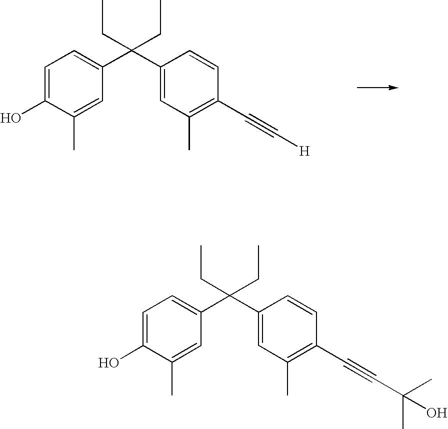

Using the same procedure as described for the preparation of Example 3, the title compound was prepared from 4-[1-ethyl-1-(4-ethynyl-3-methyl-phenyl)-propyl]-2-methyl-phenol (compound prepared in Example 1-(3)).

¹H-NMR(CDCl₃): 0.59 (t, 6H), 1.62(s,6H), 2.02(q,4H), 2.05(s, 1H), 2.18(s,3H), 2.36(s,3H), 4.60(bs, 1H), 6.63-7.00(m, 5H), 7.26(d, 1H).

### Example 6

### Preparation of 4-{1-ethyl-1-[4-(1-hydroxy-cyclobutylethynyl)-3-methyl-phenyl]-propyl}-2-methyl-phenol

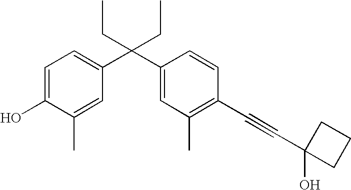

### Preparation of 4-{1-ethyl-1-[4-(1-hydroxy-cyclobutylethynyl)-3-methyl-phenyl]-propyl}-2-methyl-phenol

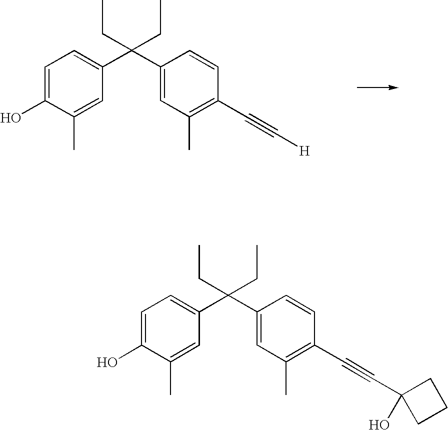

Using the same procedure as described for the preparation of Example 3, the title compound was prepared from 4-[1-ethyl-1-(4-ethynyl-3-methyl-phenyl)-propyl]-2-methyl-phenol (compound prepared in Example 1-(3)).

¹H-NMR(CDCl₃): 0.61 (t, 6H), 1.81-2.05(m, 6H), 2.01(q, 4H), 2.18(s, 3H), 2.36(s, 3H), 4.86(s, 1H), 6.65(d, 1H), 6.82(dd, 1H), 6.84(s, 1H),6.93(dd, 1H), 7.00(d,1H), 7.26(d, 1H); MS (ESI−) : 361 ([M−H]⁻).

### Example 7

### Preparation of 4-{1-ethyl-1-[4-(1-hydroxy-cyclopentylethynyl)-3-methyl-phenyl]-propyl}-2-methyl-phenol

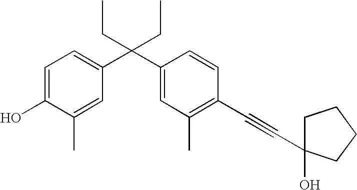

### Preparation of 4-{1-ethyl-1-[4-(1-hydroxy-cyclopentylethynyl)-3-methyl-phenyl]-propyl}-2-methyl-phenol

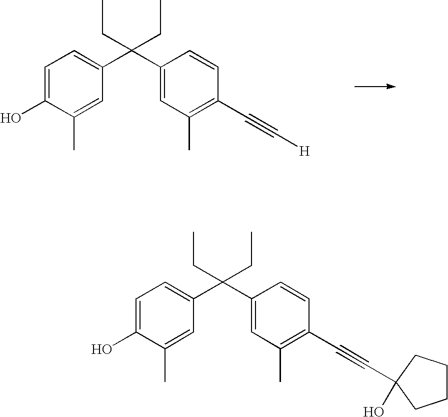

Using the same procedure as described for the preparation of Example 3, the title compound was prepared from 4-[1-ethyl-1-(4-ethynyl-3-methyl-phenyl)-propyl]-2-methyl-phenol (compound prepared in Example 1-(3)).

¹H-NMR(CDCl₃): 0.59 (t, 6H), 1.68-1.91(m,4H), 1.99˜2.10(m, 4H), 2.01(q, 4H), 2.18(s,3H), 2.36(s,3H), 4.86(s, 1H), 6.65(d, 1H), 6.82(dd, 1H), 6.84(s, 1H), 6.93(dd, 1H), 7.00(d,1H), 7.26(d, 1H); MS (ESI−) : 375 ([M−H]⁻).

### Example 8

### Preparation of 4-{1-ethyl-1-[4-(1-hydroxy-cyclohexylethynyl)-3-methyl-phenyl]-propyl}-2-methyl-phenol

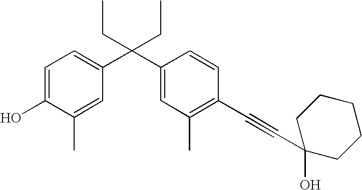

### Preparation of 4-{1-ethyl-1-[4-(1-hydroxy-cyclohexylethynyl)-3-methyl-phenyl]-propyl}-2-methyl-phenol

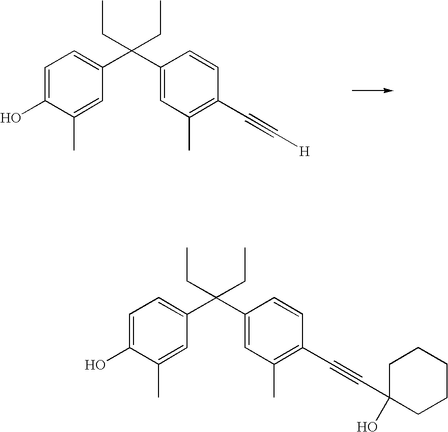

Using the same procedure as described for the preparation of Example 3, the title compound was prepared from 4-[1-ethyl-1-(4-ethynyl-3-methyl-phenyl)-propyl]-2-methyl-phenol (compound prepared in Example 1-(3)). ¹H-NMR(CDCl₃): 0.59 (t, 6H), 1.52-1.79(m, 7H), 1.92-2.08(m, 4H), 2.02(q, 4H), 2.18(s,3H), 2.38(s,3H), 4.58(s, 1H), 6.64(d, 1H), 6.84(dd, 1H), 6.86(s, 1H), 6.94(dd, 1H), 7.00(s, 1H), 7.28(d, 1H); MS (ESI−): 389 ([M−H]⁻).

### Example 9

### Preparation of 4-{1-ethyl-1-[3-methyl-4-((E)-4,4,4-trifluoro-3-hydroxy-3-trifluoromethyl-but-1-enyl)-phenyl]-propyl}-2-methyl-phenol

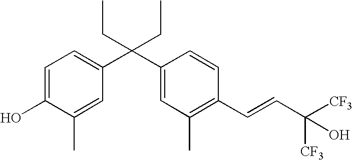

### Preparation of 4-{1-ethyl-1-[3-methyl-4-((E)-4,4,4-trifluoro-3-hydroxy-3-trifluoromethyl-but-1-enyl)-phenyl]-propyl}-2-methyl-phenol

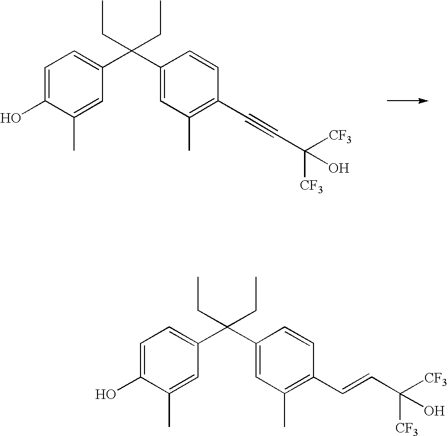

To a solution of 4-{1-ethyl-1-[3-methyl-4-(4,4,4-trifluoro-3-hydroxy-3-trifluoromethyl-but-1-ynyl)-phenyl]-propyl}-2-methyl-phenol (compound prepared in Example 2-(1)) (0.5 g, 1.09 mmol) in THF (10 ml) was slowly added LiAlH₄ (1M in THF) (4.22 ml, 4.226 mmol) at 25 degrees C. The reaction mixture was refluxed for 2 h. The reaction mixture was cooled to 0 degrees C. To the mixture, saturated aqueous NH₄Cl was added and extracted with diethyl ether. The mixture was filtered through celite, washed with brine, dried over Na₂SO₄, concentrated in vacuo, and chromatographed on silica gel (hexane/dichloromethane/ethyl acetate=1/10/1) to give the title compound (0.4 g, 80%).

¹H NMR (CDCl₃): 7.39(s, 1H), 7.34(t, 1H), 7.00(d, 1H), 6.99(s, 1H), 6.91-6.84(m, 2H), 6.65(d, 1H), 6.10(d, 1H), 4.52(s, 1H), 3.11(s, 1H), 2.33(s, 3H), 2.25(s, 3H), 2.04(q, 4H), 0.59(t, 6H).

### Example 10

### Preparation of 4-(1-ethyl-1-{4-[(E)-2-(1-hydroxy-cyclopentyl)-vinyl]-3-methyl-phenyl}-propyl)-2-methyl-phenol

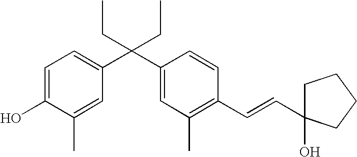

### Preparation of 4-(1-ethyl-1-{4-[(E)-2-(1-hydroxy-cyclopentyl)-vinyl]-3-methyl-phenyl}-propyl)-2-methyl-phenol

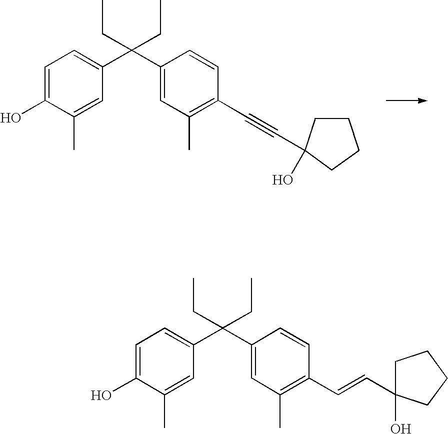

Using the same procedure as described for the preparation of Example 9, the title compound was prepared from 4-{1-ethyl-1-[4-(1-hydroxy-cyclopentylethynyl)-3-methyl-phenyl]-propyl}-2-methyl-phenol (compound prepared in Example 7).

¹H-NMR(CDCl₃): 0.60(t, 6H), 0.92(t,3H), 1.68-1.91(m,4H), 1.99-2.10(m, 4H), 2.04(q, 4H), 2.19(s,3H), 2.31(s,3H), 4.58(s, 1H), 6.26(d, 1H), 6.64(d, 1H), 6.81-6.96(m, 5H), 7.32(d, 1H); MS (ESI−): 377 ([M−H]⁻).

### Example 11

### Preparation of 4-(1-ethyl-1-{4-[(E)-2-(1-hydroxy-cyclohexyl)-vinyl]-3-methyl-phenyl}-propyl)-2-methyl-phenol

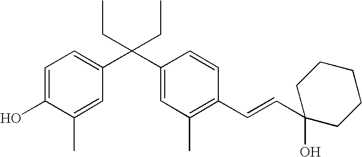

### Preparation of 4-(1-ethyl-1-{4-[(E)-2-(1-hydroxy-cyclohexyl)-vinyl]-3-methyl-phenyl}-propyl)-2-methyl-phenol

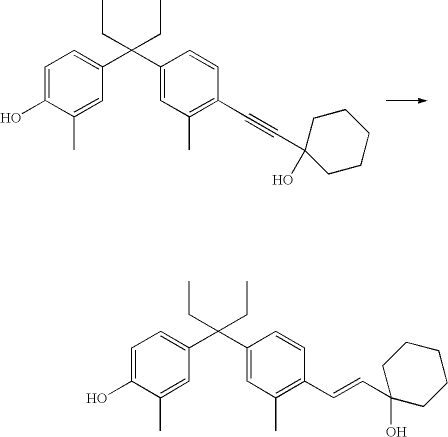

Using the same procedure as described for the preparation of Example 9, the title compound was prepared from 4-{1-ethyl-1-[4-(1-hydroxy-cyclohexylethynyl)-3-methyl-phenyl]-propyl}-2-methyl-phenol (compound prepared in Example 8).

¹H-NMR(CDCl₃): 0.60 (t, 6H), 1.33-1.74(m, 10H), 2.04(q, 4H), 2.23(s,3H), 2.31 (s,3H), 4.68(s, 1H), 6.23(d, 1H), 6.65(d, 1H), 6.79-7.00(m, 5H),7.31(d, 1H); MS (ESI−): 391 ([M−H]⁻).

### Example 12

### Preparation of 4-{1-ethyl-1-[4-((E)-3-hydroxy-3-methyl-but-1-enyl)-3-methyl-phenyl]-propyl}-2-methyl-phenol

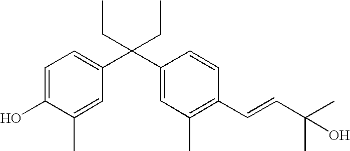

### Preparation of 4-{1-ethyl-1-[4-((E)-3-hydroxy-3-methyl-but-1-enyl)-3-methyl-phenyl]-propyl}-2-methyl-phenol

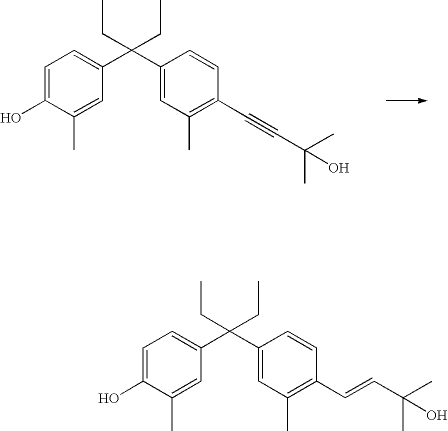

Using the same procedure as described for the preparation of Example 9, the title compound was prepared from 4-{1-ethyl-1-[4-(3-hydroxy-3-methyl-but-1-ynyl)-3-methyl-phenyl]-propyl}-2-methyl-phenol (compound prepared in Example 5).

¹H-NMR(CDCl₃): 0.60 (t, 6H), 1.42(s, 6H), 2.05(q, 4H), 2.18(s, 3H), 2.30(s, 3H), 4.97(s, 1H), 6.22(d, 1H), 6.64(d, 1H), 6.76(d, 1H), 6.84-6.90(m, 2H), 6.94-6.97(m, 2H), 7.31(d, 1H); MS (ESI−): 351 ([M−H]⁻).

### Example 13

### Preparation of 4-{1-ethyl-1-[4-((E)-3-hydroxy-3-propyl-hex-1-enyl)-3-methyl-phenyl]-propyl}-2-methyl-phenol

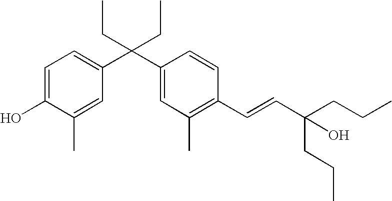

### Preparation of 4-{1-ethyl-1-[4-((E)-3-hydroxy-3-propyl-hex-1-enyl)-3-methyl-phenyl]-propyl}-2-methyl-phenol

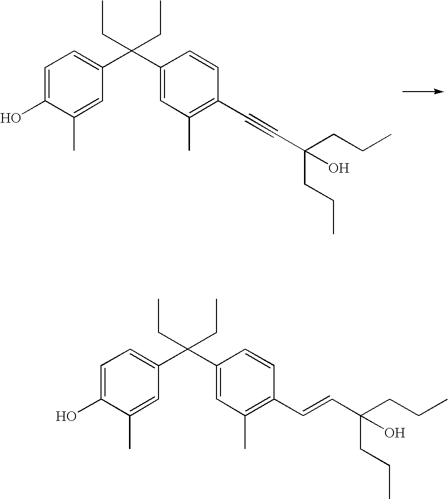

Using the same procedure as described for the preparation of Example 9, the title compound was prepared from 4-{1-ethyl-1-[4-(3-hydroxy-3-propyl-hex-1-ynyl)-3-methyl-phenyl]-propyl}-2-methyl-phenol (compound prepared in Example 4).

¹H-NMR(CDCl₃): 0.61 (t, 6H), 0.92(t, 3H), 1.32˜1.50(m, 3H), 1.54˜1.64(m, 5H), 2.03(q, 4H), 2.19(s,3H), 2.30(s,3H), 4.71(s, 1H), 6.06(d, 1H), 6.69(d, 1H), 6.75(d, 1H), 6.85˜6.96(m, 4H), 7.32(d, 1H); MS (ESI−): 407 ([M−H]⁻).

### Example 14

### Preparation of 4-{1-ethyl-1-[4-((E)-3-hydroxy-4,4-dimethyl-pent-1-enyl)-3-methyl-phenyl]-propyl}-2-methyl-phenol

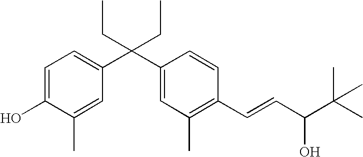

### Preparation of 4-{1-ethyl-1-[4-((E)-3-hydroxy-4,4-dimethyl-pent-1-enyl )-3-methyl-phenyl]-propyl}-2-methyl-phenol

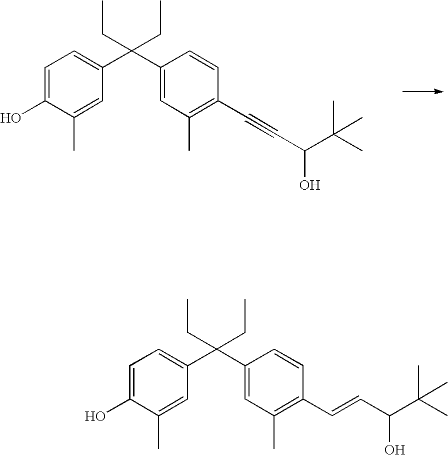

Using the same procedure as described for the preparation of Example 9, the title compound was prepared from 4-{1-ethyl-1-[4-(3-hydroxy-4,4-dimethyl-pent-1-ynyl)-3-methyl-phenyl]-propyl}-2-methyl-phenol (compound prepared in Example 3).

¹H-NMR(CDCl₃): 0.60 (t, 6H), 0.96 (s, 9H), 2.03(q, 4H), 2.19(s, 3H), 2.29(s, 3H), 3.92(d, 1H), 6.12(dd, 1H), 6.65(d, 1H), 6.73(d, 1 H), 6.84-6.97(m,4H), 7.31(d, 1H); MS (ESI−): 379 ([M−H]⁻).

### Example 15

### Preparation of 4-(1-ethyl-1-{4-[(E)-2-(1-hydroxy-cyclobutyl)-vinyl]-3-methyl-phenyl}-propyl)-2-methyl-phenol

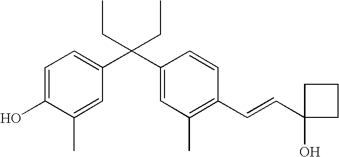

### Preparation of 4-(1-ethyl-1-{4-[(E)-2-(1-hydroxy-cyclobutyl)-vinyl]-3-methyl-phenyl}-propyl)-2-methyl-phenol

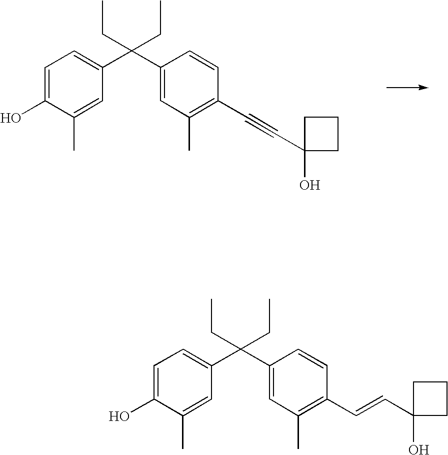

Using the same procedure as described for the preparation of Example 9, the title compound was prepared from 4-{1-ethyl-1-[4-(1-hydroxy-cyclobutylethynyl)-3-methyl-phenyl]-propyl}-2-methyl-phenol (compound prepared in Example 6).

¹H-NMR(CDCl₃): 0.61 (t, 6H), 1.54-1.91(m, 3H), 2.05(q, 4H), 2.18(s,3H), 2.30(m, 3H), 2.32(s, 3H), 4.75(s, 1H), 6.47(d, 1H), 6.68(d, 1H), 6.81-7.00(m, 5H), 7.39(d, 1H).

### Example 16

### Preparation of 2-chloro-4-{1-[3-chloro-4-(3-hydroxy-4,4-dimethyl-pentyl)-phenyl]-1-ethyl-propyl}-phenol (Enantiomer 1)

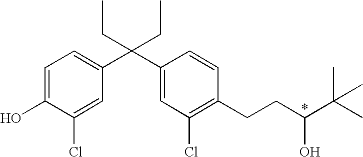

### (1) Preparation of trifluoro-methanesulfonic acid 2-chloro-4-[1-(3-chloro-4-hydroxy-phenyl)-1-ethyl-propyl]-phenyl ester

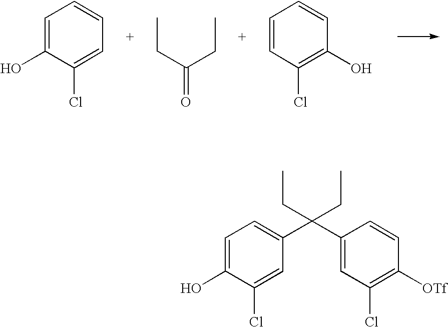

To a mixture of 2-chloro-phenol (12.86 g, 100 mmol) and 3-pentanone (5.30 ml, 50 mmol), trifluoromethane sulfonic acid (3.1 ml, 35 mmol) was added and stirred for 17 hr at 90 degrees C. The reaction mixture was chromatographed on silica gel (ethyl acetate/hexane=0/100 to 17/83) to give the brown foam (324 mg). The foam was dissolved with CH₂Cl₂ (5 ml) and pyridine (0.097 ml, 1.2 mmol), trifluoromethane sulfonic anhydride (0.185 ml, 1.1 mmol) was added and stirred for 1.5 h at 0 degrees C. To the reaction mixture, aqueous KHSO₄ was added and extracted with ethyl acetate, washed with brine, dried over MgSO₄, concentrated in vacuo, and chromatographed on silica gel (ethyl acetate/hexane=0/100 to 18/82) to give the title compound (117 mg, 0.5%).

¹H-NMR: 0.63 (t, 6H), 2.04 (q, 4H), 5.46 (s, 1H), 6.90-7.34 (m, 6H); MS(ESI−): 455([M−H]⁻).

### (2) Preparation of 2-chloro-4-[1-(3-chloro-4-trimethylsilanylethynyl-phenyl)-1-ethyl-propyl]-phenol

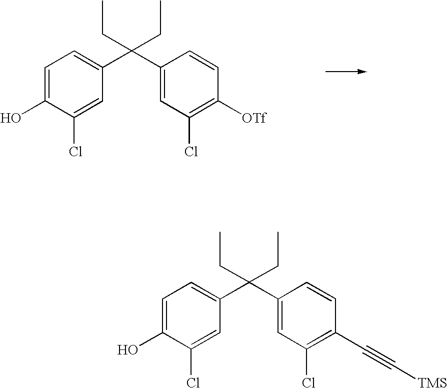

To a solution of trifluoro-methanesulfonic acid 2-chloro-4-[1-(3-chloro-4-hydroxy-phenyl)-1-ethyl-propyl]-phenyl ester (compound prepared in Example 16-(1)) (117 mg, 0.256 mmol) in MeCN (1 ml) was added trimethylsilylacethylene (0.109 ml, 0.768 mmol), Pd(PPh₃)₄ (30 mg, 0.026 mmol), Cul (5 mg, 0.026 mmol) and triethylamine (0.107 mL, 0.768 mmol) were added and stirred for 1 h at 100 degrees C. under nitrogen atmosphere. To the reaction mixture, saturated aqueous NH₄Cl was added and extracted with ethyl acetate, washed with H₂O twice, dried over MgSO₄, concentrated in vacuo, and chromatographed on silica gel (ethyl acetate/hexane=0/100 to 15/85) to give the title compound (27 mg, 26%).

¹H-NMR: 0.26 (s, 9H), 0.61 (t, 6H), 2.01 (q, 4H), 5.35-5.45 (brs, 1H), 6.88-7.39 (m, 6H).

### (3) Preparation of 2-chloro-4-[1-(3-chloro-4-ethynyl-phenyl)-1-ethyl-propyl]-phenol

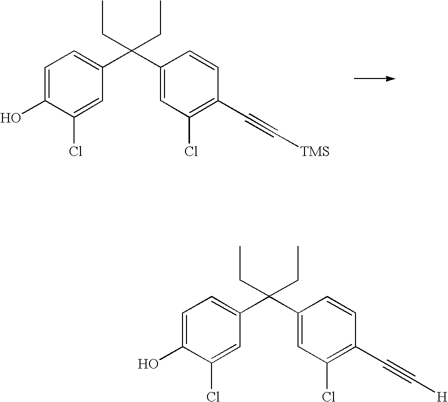

To a solution of 2-chloro-4-[1-(3-chloro-4-trimethylsilanylethynyl-phenyl)-1-ethyl-propyl]-phenol (compound prepared in Example 16-(2)) (27 mg, 0.067 mmol) in THF (0.5 ml) was added 1M TBAF in THF (0.33 ml, 0.33 mmol) at room temperature. The reaction mixture was allowed for 3 min. To the reaction mixture, aqueous KHSO₄ was added and extracted with ethyl acetate/hexane=1/1, washed with aqueous NaHCO₃, aqueous KHSO₄, aqueous NaHCO₃, aqueous KHSO₄, aqueous NaHCO₃, successively, dried over MgSO₄, concentrated in vacuo to give the title compound (20 mg, 90%).

¹H-NMR: 0.62 (t, 6H), 2.03 (q, 4H), 3.33 (s, 1H), 6.90-7.41 (m, 6H).

### (4) Preparation of 2-chloro-4-{14-[3-chloro-4-(3-hydroxy-4,4-dimethyl-pent-1-ynyl)-phenyl]-1-ethyl-propyl}-phenol

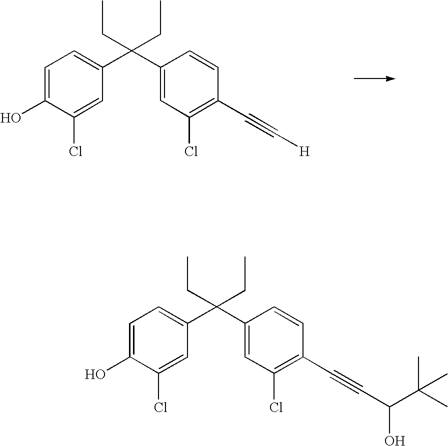

To a solution of 2-chloro-4-[1-(3-chloro-4-ethynyl-phenyl)-1-ethyl-propyl]-phenol (compound prepared in Example 16-(3)) (20 mg, 0.06 mmol) in THF (0.6 ml) was added 1.59 M n-butyl lithium in hexane (0.094 ml, 0.15 mmol) at 0 degrees C. The reaction mixture was stirred for 30 min at 0 degrees C. trimethylacetaldehyde (0.020 ml, 0.18 mmol) was added, stirred at 0 degrees C. for 30 min. To the reaction mixture, saturated aqueous NH₄Cl was added and extracted with ethyl acetate, washed with brine, dried over MgSO₄, concentrated in vacuo, and chromatographed on silica gel (ethyl acetate/hexane=1/5) to give the title compound (15 mg, 60%).

¹H-NMR: 0.62 (t, 6H), 1.08 (s, 9H), 2.02 (q, 4H), 4.26 (s, 1H), 6.90-6.98 (m, 3H), 7.09-7.21 (m, 2H), 7.35 (d, 1H).

### (5) Chiral separation of 2-chloro-4-{1-[3-chloro-4-(3-hydroxy4,4-dimethyl-pent-1-ynyl)-phenyl]-1-ethyl-propyl}-phenol

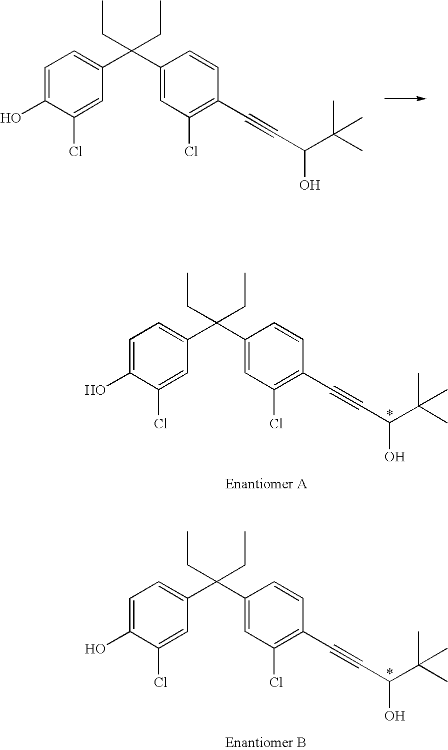

Racemic mixture of 2-chloro-4-{1-[3-chloro-4-(3-hydroxy-4,4-dimethyl-pent-1-ynyl)-phenyl]-1-ethyl-propyl}-phenol (compound prepared in Example 16-(4)) (15 mg, 0.036 mmol) was separated with HPLC (CHIRALPAK OJ [DAICEL, 20 mm I.D., 250 mm], Ethanol/hexane=15/85), to give each enantiomer (Enantiomer A: 6 mg/Enantiomer B: 6 mg).
Enantiomer A
Column: CHIRALPAK OJ-RH 4.6×150 mm (DAICEL)
Eluent: Hexane/EtOH=85/15
Temperature: 35 degrees C.
Flow rate: 1.0 ml/min.
Retention time: 4.1 min.
Enantiomer B
Retention time: 6.1 min.

### (6) Preparation of 2-chloro-4-{1-[3-chloro-4-(3-hydroxy-4,4-dimethyl-pentyl)-phenyl]-1-ethyl-propyl}-phenol (Enantiomer 1)

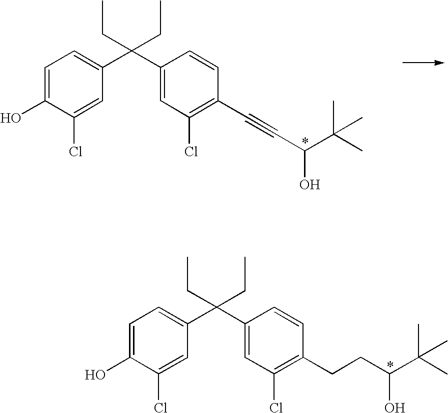

To a solution of 2-chloro-4-{1-[3-chloro-4-(3-hydroxy-4,4-dimethyl-pent-1-ynyl)-phenyl]-1-ethyl-propyl}-phenol (Enantiomer A prepared in Example 16-(5)) (6 mg, 0.014 mmol) in ethyl acetate (1 ml), 10% Pd-C (1 mg) was added and the mixture was stirred under hydrogen atmosphere for 90 min. The reaction mixture was filtered and concentrated in vacuo to give the title compound (6 mg, 99%).

¹H-NMR: 0.62 (t, 6H), 0.89 (s, 9H), 1.43-1.59 (m, 1H), 1.81-1.92 (m, 1H), 2.02 (q, 4H), 2.66-2.76 (m, 1H), 2.92-3.02 (m, 1H), 3.21-3.25 (m, 1H), 5.40 (s, 1H), 6.88-6.95 (m, 3H), 7.10-7.15 (m, 3H).

### Example 17

### Preparation of 2-chloro-4-{1-[3-chloro-4-(3-hydroxy-4,4-dimethyl-pentyl)-phenyl]-1-ethyl-propyl}-phenol (Enantiomer 2)

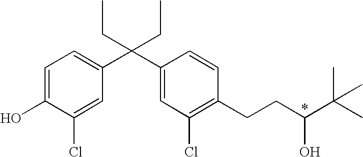

### Preparation of 2-chloro-4-{1-[3-chloro-4-(3-hydroxy-4,4-dimethyl-pentyl)-phenyl]-1-ethyl-propyl}-phenol (Enantiomer 2)

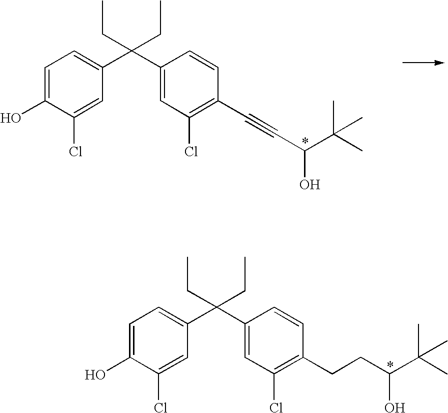

To a solution of 2-chloro-4-{1-[3-chloro-4-(3-hydroxy-4,4-dimethyl-pent-1-ynyl)-phenyl]-1-ethyl-propyl}-phenol (Enantiomer B prepared in Example 16-(5)) (6 mg, 0.014 mmol) in ethyl acetate (1 ml), 10% Pd-C (1 mg) was added and the mixture was stirred under hydrogen atmosphere for 90 min. The reaction mixture was filtered and concentrated in vacuo to give the title compound (6 mg, 99%).

¹H-NMR: 0.62 (t, 6H), 0.89 (s, 9H), 1.43-1.59 (m, 1H), 1.81-1.92 (m, 1H), 2.02 (q, 4H), 2.66-2.76 (m, 1H), 2.92-3.02 (m, 1H), 3.21-3.25 (m, 1H), 5.40 (s, 1H), 6.88-6.95 (m, 3H), 7.10-7.15 (m, 3H).

### Example 17

### Preparation of 2-chloro-4-{1-[3-chloro-4-(3-hydroxy-4,4-dimethyl-pentyl)-phenyl]-1-ethyl-propyl}-phenol (Enantiomer 2)

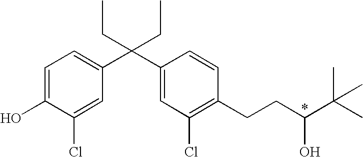

### Preparation of 2-chloro-4-{1-[3-chloro-4-(3-hydroxy-4,4-dimethyl-pentyl)-phenyl]-1-ethyl-propyl}-phenol (Enantiomer 2)

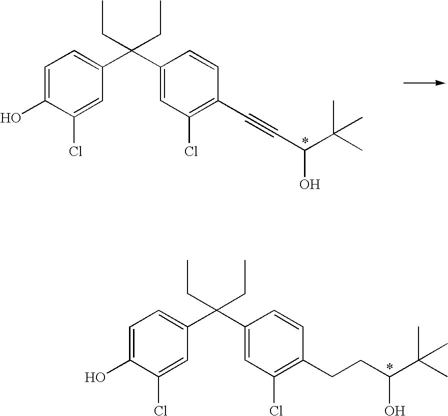

To a solution of 2-chloro-4-{1-[3-chloro-4-(3-hydroxy-4,4-dimethyl-pent-1-ynyl)-phenyl]-1-ethyl-propyl}-phenol (Enantiomer B prepared in Example 16-(5)) (6 mg, 0.014 mmol) in ethyl acetate (1 ml), 10% Pd-C (1 mg) was added and stirred under hydrogen atmosphere for 90 min. The reaction mixture was filtered and concentrated under vacuum to give the title compound (6 mg, 99%).

¹H-NMR: 0.62 (t, 6H), 0.89 (s, 9H), 1.43-1.59 (m, 1H), 1.81-1.92 (m, 1H), 2.02 (q, 4H), 2.66-2.76 (m, 1H), 2.92-3.02 (m, 1H), 3.21-3.25 (m, 1H), 5.40 (s, 1H), 6.88-6.95 (m, 3H), 7.10-7.15 (m, 3H).

### Example 18

### Preparation of 4-{1-ethyl-1-[4-(3-hydroxy-4,4-dimethyl-pentyl)-3-methyl-phenyl]-propyl}-2-methyl-phenol

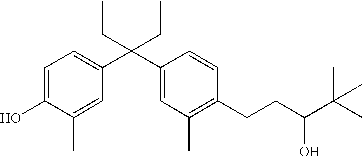

### (1) Preparation of (E)-3-{4-[1-ethyl-1-(4-hydroxy-3-methyl-phenyl)-propyl]-2-methyl-phenyl}-acrylic acid methyl ester

Trifluoromethanesulfonic acid 4-[1-ethyl-1-(4-hydroxy-3-methyl-phenyl)-propyl]-2-methyl-phenyl ester (compound prepared in Example 1-(1)) (10 g, 24.0 mmol) was dissolved in anhydrous DMF (70 ml) under nitrogen atmosphere. To this solution were added triethylamine (4.0 ml, 28.8 mmol), methylacrylate (2.6 ml, 28.8 mmol), 1,3-bis(diphenylphosphine)propane (544 mg, 1.32 mmol) and palladium acetate (269 mg, 1.2 mmol) at room temperature. After stirring 1.5 h at 100 degrees C., the resulting solution was diluted with CH₂Cl₂, and washed with 5% HCl solution and water until neutrality was achieved, and dried over Na₂SO₄. The solution was evaporated under reduced pressure and purified by silica-gel column (hexane/ethyl acetate=7:1) to yield the title compound (7.6 g, 21.5 mmol, 90%).

¹H NMR (CDCl₃): 7.94 (d, 1H), 7.42 (d, 1H), 7.00 (m, 2H), 6.85 (m, 2H), 6.65 (d, 1H), 6.32 (d, 1H), 4.70 (s, phenol), 3.79 (s, 3H), 2.38 (s, 3H), 2.19 (s, 3H), 2.04 (q, 4H), 0.60 (t, 6H).

### (2) Preparation of 3-{4-[1-ethyl-1-(4-hydroxy-3-methyl-phenyl)-propyl]-2-methyl-phenyl}-propionic acid methyl ester

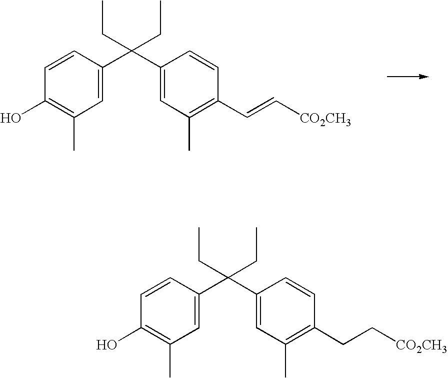

(E)-3-{4-[1-Ethyl-1-(4-hydroxy-3-methyl-phenyl)-propyl]-2-methyl-phenyl}-acrylic acid methyl ester (compound prepared in Example 18-(1)) (270 mg, 0.76 mmol) was dissolved in methanol (7 ml). To this solution was added palladium carbon (10 mg), and stirred at room temperature under hydrogen gas overnight. The resulting solution was filtered through celite, and the filtrate was evaporated under reduced pressure and purified by silica-gel chromatography (hexane/ethyl acetate=10:1) to give the title compound (266 mg, 0.75 mmol, 98%).

¹H NMR (CDCl₃): 6.99-6.83 (m, 5H), 6.64 (d, 1H), 4.54 (s, phenol), 3.67 (s, 3H), 2.89 (dt, 2H), 2.57 (dt, 2H), 2.24 (s, 3H), 2.19 (s, 3H), 2.02 (q, 4H), 0.59 (t, 6H).

### (3) Preparation of 1-{4-[1-ethyl-1-(4-hydroxy-3-methyl-phenyl)-propyl]-2-methyl-phenyl}-4,4-dimethyl-pentan-3-one

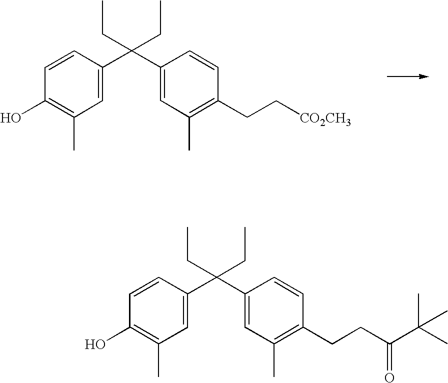

3-{4-[1-Ethyl-1-(4-hydroxy-3-methyl-phenyl)-propyl]-2-methyl-phenyl}-propionic acid methyl ester (compound prepared in Example 18-(2)) (3.0 g, 8.46 mmol) was dissolved in anhydrous THF (80 ml) under nitrogen atmosphere. The solution was cooled to −78 degrees C. To this solution was added t-BuLi (4.97 ml, 8.46 mmol, 1.7M in pentane) at −78 degrees C. After stirring 30 min at −78 degrees C., the solution was diluted with dichloromethane, and washed with saturated NH₄Cl aq., water and brine, and dried over Na₂SO₄. The solution was evaporated under reduced pressure and purified by silica-gel chromatography (hexane/ethyl acetate=7:1) to give the title compound (2.5 g, 6.56 mmol, 77%).

¹H NMR (CDCl₃): 7.03-6.83 (m, 5H), 6.64 (d,1 H), 4.60 (s, phenol), 2.82 (dt, 2H), 2.71 (dt, 2H), 2.24 (s, 3H), 2.18 (s, 3H), 2.04 (q, 4H), 1.09 (s, 9H), 0.58 (t, 6H).

### (4) Preparation of 4-{1-ethyl-1-[4-(3-hydroxy-4,4-dimethyl-pentyl)-3-methyl-phenyl]-propyl}-2-methyl-phenol

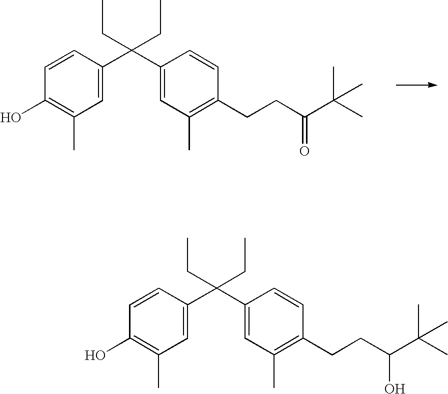

1-{4-[1-Ethyl-1-(4-hydroxy-3-methyl-phenyl)-propyl]-2-methyl-phenyl}-4,4-dimethyl-pentan-3-one (compound prepared in Example 18-(3)) (200 mg, 0.52 mmol) was dissolved in methanol (5 ml) under nitrogen atmosphere. To this solution was added sodium borohydride (29.8 mg, 0.78 mmol) at 0 degrees C. After stirring 1.0 h at room temperature, the solution was evaporated under reduced pressure and the obtained residue was purified by silica-gel chromatography (hexane/ethyl acetate=7:1) to give the title compound (190 mg, 0.50 mmol, 95%).

¹H NMR (CDCl₃): 7.03-6.83 (m, 5H), 6.63 (d, 1H), 4.99 (s, phenol), 3.25 (dt, 1H), 2.86 (m, 1H), 2.55 (m, 1H), 2.24 (s, 3H), 2.18 (s, 3H), 2.03 (q, 4H), 1.79 (m,1H), 1.50 (m, 1H), 0.89 (s, 9H), 0.59 (t, 6H).

### Example 19

### Preparation of 4-{1-ethyl-1-[4-(3-hydroxy-4,4-dimethyl-pentyl)-3-methyl-phenyl]-propyl}-2-methyl-phenol (Enantiomer 1)

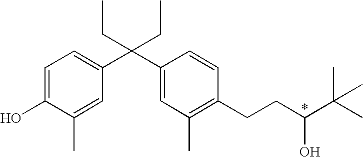

### (1) Chiral separation of 4-{1-ethyl-1-[4-(3-hydroxy-4,4-dimethyl-pent-1-ynyl)-3-methyl-phenyl]-propyl}-2-methyl-phenol

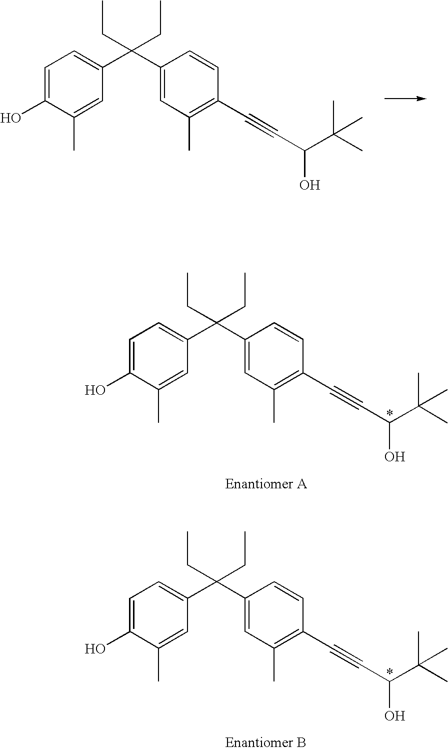

Racemic mixture of 4-{1-ethyl-1-[4-(3-hydroxy-4,4-dimethyl-pent-1-ynyl)-3-methyl-phenyl]-propyl}-2-methyl-phenol (compound prepared in Example 3) (13.08 g, 34.6 mmol) was separated with HPLC (CHIRALPAK AD [DAICEL, 20 mm I.D., 250 mm], 2-propanol/hexane=15/85), to give each enantiomer (Enantiomer A: 99.9% ee, 5.58 g/Enantiomer B: 5.00 g, 99.4% ee).
Enantiomer A
Column: CHIRALPAK AD-H 4.6×150 mm (DAICEL)
Eluent: Hexane/2-propanol=85/15
Flow rate: 1.0 ml/min.
Retention time: 5.1 min.
Enantiomer B
Retention time: 7.2 min.

### (2) Preparation of 4-{1-ethyl-1-[4-(3-hydroxy-4,4-dimethyl-pentyl)-3-methyl-phenyl]-propyl}-2-methyl-phenol (Enantiomer 1)

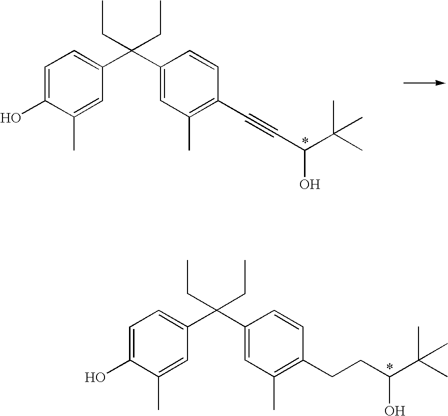

To a solution of 4-{1-ethyl-1-[4-(3-hydroxy-4,4-dimethyl-pent-1-ynyl)-3-methyl-phenyl]-propyl}-2-methyl-phenol (Enantiomer A prepared in Example 19-(1)) (796 mg, 2.1 mmol) in ethyl acetate (10 ml), 10% Pd-C (0.1 g) was added and stirred under hydrogen atmosphere for 40 min. The reaction mixture was filtered and concentrated in vacuo to give the title compound (732 mg, 91%).

¹H-NMR: 0.60 (t, 6H), 0.89 (s, 9H), 1.40 (d, 1H), 1.44-1.55 (m, 1H), 1.74-1.85 (m, 1H), 2.03 (q, 4H), 2.19 (s, 3H), 2.25 (s, 3H), 2.51-2.61 (m, 1H), 2.81-2.91 (m, 1H), 3.25 (dd, 1H), 4.59 (s, 1H), 6.64 (d, 1H), 6.85-7.00 (m, 4H), 7.03 (d, 1H).

### Example 20

### Preparation of 4-{1-ethyl-1-[4-(3-hydroxy-4,4-dimethyl-pentyl)-3-methyl-phenyl]-propyl}-2-methyl-phenol (Enantiomer 2)

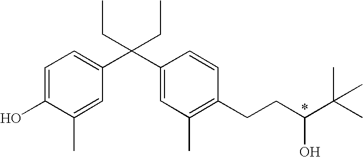

### Preparation of 4-{1-ethyl-1-[4-(3-hydroxy-4,4-dimethyl-pentyl)-3-methyl-phenyl]-propyl}-2-methyl-phenol (Enantiomer 2)

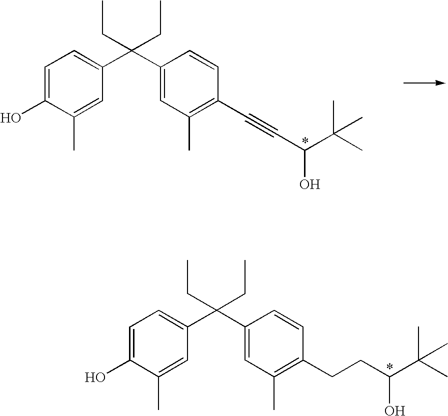

To a solution of 4-{1-ethyl-1-[4-(3-hydroxy-4,4-dimethyl-pent-1-ynyl)-3-methyl-phenyl]-propyl}-2-methyl-phenol (Enantiomer B prepared in Example 19-(1)) (725 mg, 1.92 mmol) in ethyl acetate (10 ml), 10% Pd-C (0.1 g) was added and stirred under hydrogen atmosphere for 40 min. The reaction mixture was filtered and concentrated in vacuo to give the title compound (720 mg, 98%).

¹H-NMR: 0.60 (t, 6H), 0.89 (s, 9H), 1.40 (d, 1H), 1.44-1.55 (m, 1H), 1.74-1.85 (m, 1H), 2.03 (q, 4H), 2.19 (s, 3H), 2.25 (s, 3H), 2.51-2.61 (m, 1H), 2.81-2.91 (m, 1H), 3.25 (dd, 1H), 4.59 (s, 1H), 6.64 (d, 1H), 6.85-7.00 (m, 4H), 7.03 (d, 1H).

### Example 21

### Preparation of (S)-5-(4-{1-ethyl-1-[4-(3-hydroxy-3-methyl-but-1-ynyl)-3-methyl-phenyl]-propyl}-2-methyl-phenoxy)-4-hydroxy-pentanoic acid

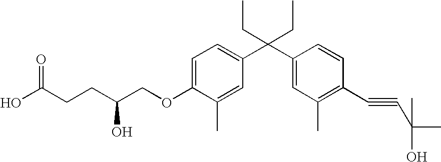

### (1) Preparation of (S)-5-(4-{1-ethyl-1-[4-(3-hydroxy-3-methyl-but-1-ynyl)-3-methyl-phenyl]-propyl}-2-methyl-phenoxymethyl)-dihydro-furan-2-one

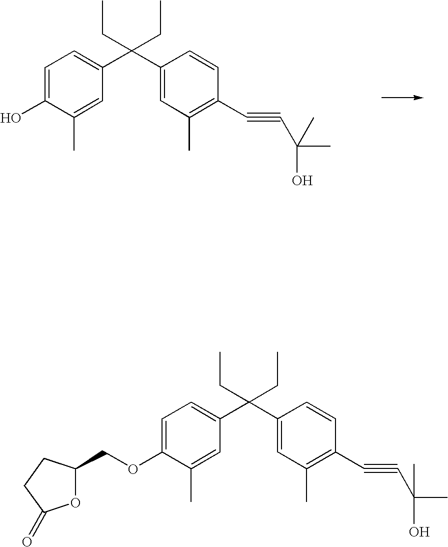

Using the same procedure as described for the preparation of Example 1-(6), the title compound was prepared from 4-{1-ethyl-1-[4-(3-hydroxy-3-methyl-but-1-ynyl)-3-methyl-phenyl]-propyl}-2-methyl-phenol (compound prepared in Example 5). The yield was 58%.

¹H-NMR (300 MHz, CDCl₃): 0.60 (t, 6H), 1.57 (s, 6H), 2.09 (q, 4H), 2.16 (s, 3H), 2.26 (m, 1H), 2.34 (s, 3H), 2.39-2.62 (m, 3H), 2.71-2.82 (m, 1H), 4.06 (dd, 1H), 4.14 (dd, 1H), 4.84-4.91 (m, 1H), 6.69 (d, 1H), 6.89 (m, 2H), 6.95 (d, 1H), 7.00 (s, 1H), 7.27 (d, 1H).

### (2) Preparation of (S)-5-(4-{1-ethyl-1-[4-(3-hydroxy-3-methyl-but-1-ynyl)-3-methyl-phenyl]-propyl}-2-methyl-phenoxy)-4-hydroxy-pentanoic acid

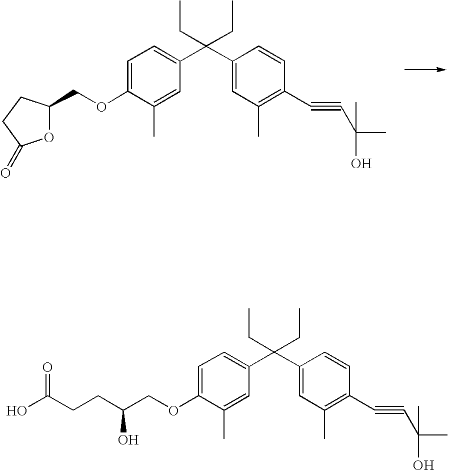

Using the same procedure as described for the preparation of Example 1-(7), the title compound was prepared from (S)-5-(4-{1-ethyl-1-[4-(3-hydroxy-3-methyl-but-1-ynyl)-3-methyl-phenyl]-propyl}-2-methyl-phenoxymethyl)-dihydro-furan-2-one (compound prepared in Example 21-(1)). The yield was 71%.

¹H-NMR (300 MHz, CD₃OD as potassium salt): 0.60 (t, 6H), 1.57 (s, 6H), 1.79-2.02 (m, 2H), 2.04 (q, 4H), 2.16 (s, 3H), 2.34 (s, 3H), 2.35 (td, 2H), 3.90-3.93 (m, 3H), 6.77 (d, 1H), 6.84 (s, 1H), 6.94 (dd, 2H), 7.00 (s, 1H), 7.21 (d, 1H); MS(ESI−): m/z: 465 ([M−H]⁻).

### Example 22

### Preparation of (S)-5-(4-{1-ethyl-1-[4-((E)-3-hydroxy-3-methyl-but-1-enyl)-3-methyl-phenyl]-propyl}-2-methyl-phenoxy)-4-hydroxy-pentanoic acid

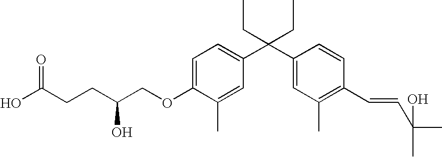

### (1) Preparation of (S)-5-(4-{1-ethyl-1-[4-((E)-3-hydroxy-3-methyl-but-1-enyl)-3-methyl-phenyl]-propyl}-2-methyl-phenoxymethyl)-dihydro-furan-2-one

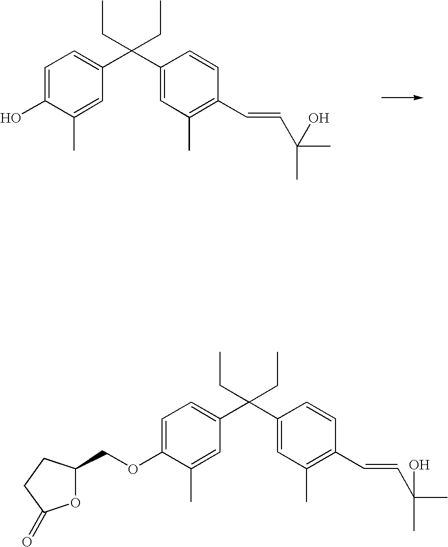

Using the same procedure as described for the preparation of Example 1-(6), the title compound was prepared from 4-{1-ethyl-1-[4-((E)-3-hydroxy-3-methyl-but-1-enyl)-3-methyl-phenyl]-propyl}-2-methyl-phenol (compound prepared in Example 12). The yield was 44%.

¹H-NMR (300 MHz, CDCl₃): 0.57 (t, 6H), 1.42 (s, 6H), 2.05 (q, 4H), 2.20 (s, 3H), 2.29 (m, 1H), 2.34 (s, 3H), 2.39-2.62 (m, 3H), 2.71-2.82 (m, 1H), 4.06 (dd, 1H), 4.14 (dd, 1H), 4.84-4.91 (m, 1H), 6.16 (d, 1H), 6.72 (s, 1H), 6.76 (d, 1H), 6.83 (s, 1H), 6.91-6.95 (m, 3H), 7.32 (d, 1H).

### (2) Preparation of (S)-5-(4-{1-ethyl-1-[4-((E)-3-hydroxy-3-methyl-but-1-enyl)-3-methyl-phenyl]-propyl}-2-methyl-phenoxy)-4-hydroxy-pentanoic acid

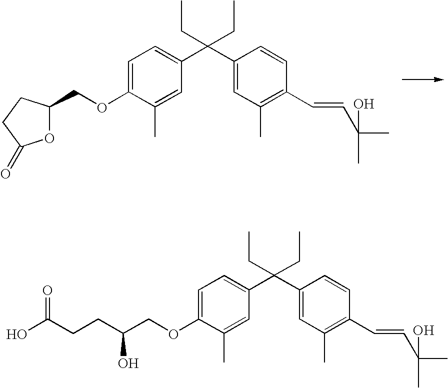

Using the same procedure as described for the preparation of Example 1-(7), the title compound was prepared from (S)-5-(4-{1-ethyl-1-[4-((E)-3-hydroxy-3-methyl-but-1-enyl)-3-methyl-phenyl]-propyl}-2-methyl-phenoxymethyl)-dihydro-furan-2-one (compound prepared in Example 22-(1)). The yield was 90%.

¹H-NMR (300 MHz, CD₃OD as potassium salt): 0.60 (t, 6H), 1.57 (s, 6H), 1.82-2.01 (m, 2H), 2.05 (q, 4H), 2.13 (s, 3H), 2.25 (s, 3H), 2.37 (td, 2H), 3.90-3.93 (m, 3H), 6.16 (d, 1H), 6.72 (s, 1H), 6.76 (d, 1H), 6.83 (s, 1H), 6.90-6.95 (m, 3H), 7.27 (d, 1H); MS(ESI−): 467 ([M−H]⁻).

### Example 23

### Preparation of (S)-5-(4-{1-ethyl-1-[4-(3-ethyl-3-hydroxy-pent-1-ynyl)-3-methyl-phenyl]-propyl}-2-methyl-phenoxy)-4-hydroxy-pentanoic acid

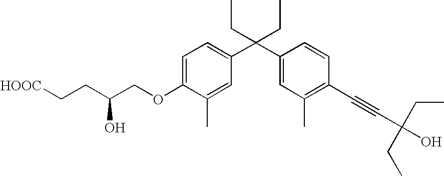

### (1) Preparation of (S)-5-(4-{1-ethyl-1-[4-(3-ethyl-3-hydroxy-pent-1-ynyl)-3-methyl-phenyl]-propyl}-2-methyl-phenoxymethyl)-dihydro-furan-2-one

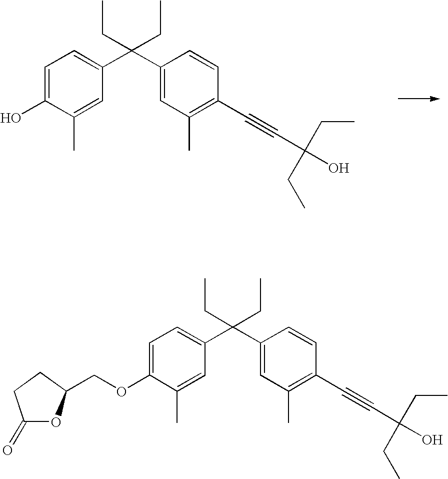

Using the same procedure as described for the preparation of Example 1-(6), the title compound was prepared from 4-{1-ethyl-1-[4-(3-ethyl-3-hydroxy-pent-1-ynyl)-3-methyl-phenyl]-propyl}-2-methyl-phenol (compound prepared in Example 1-(4)). The yield was 56%.

¹H-NMR (300 MHz, CDCl₃): 0.59 (t, 6H), 1.11 (t, 6H), 1.72-1.81 (q, 4H), 2.03 (q, 4H), 2.14 (s, 3H), 2.37 (s, 3H), 2.28-2.62 (m, 3H), 2.72-2.85 (m, 1H), 4.04-4.19 (m, 2H), 4.89 (m, 1H), 6.65 (d, 1H), 6.87 (d, 2H), 6.91 (s, 1H), 6.98 (s, 1H), 7.27 (d, 1H).

### (2) Preparation of (S)-5-(4-{1-ethyl-1-[4-(3-ethyl-3-hydroxy-pent-1-ynyl)-3-methyl-phenyl]-propyl}-2-methyl-phenoxy)-4-hydroxy-pentanoic acid

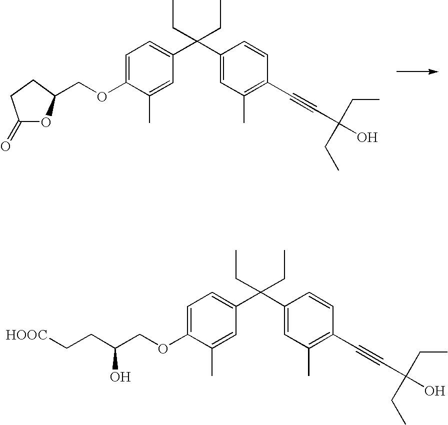

Using the same procedure as described for the preparation of Example 1-(7), the title compound was prepared from (S)-5-(4-{1-ethyl-1-[4-(3-ethyl-3-hydroxy-pent-1-ynyl)-3-methyl-phenyl]-propyl}-2-methyl-phenoxymethyl)-dihydro-furan-2-one (compound prepared in Example 23-(1)). The yield was 76%.

¹H-NMR (300 MHz, CDCl₃): 0.59 (t, 6H), 1.11 (t, 6H), 1.72-1.81 (m, 4H), 1.87-1.98 (m, 2H), 2.03 (q, 4H), 2.16 (s,3H), 2.37 (s,3H), 2.62 (t, 2H), 3.82-3.87 (dd, 1H), 3.95-3.99 (dd, 1H), 4.05-4.11 (m, 1H), 6.67 (d, 1H), 6.86 (s, 1H), 6.90 (s, 1H), 6.94 (d,1H), 6.99 (d, 1H), 7.27 (d, 1H); MS(ESI−): 493 ([M−H]⁻).

### Example 24

### Preparation of (S)-5-(4-{1-ethyl-1-[4-(3-ethyl-3-hydroxy-pentyl)-3-methyl-phenyl]-propyl}-2-methyl-phenoxy)-4-hydroxy-pentanoic acid

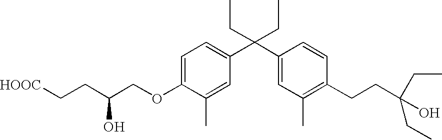

### Preparation of (S)-5-(4-{1-ethyl-1-[4-(3-ethyl-3-hydroxy-pentyl)-3-methyl-phenyl]-propyl}-2-methyl-phenoxy)-4-hydroxy-pentanoic acid

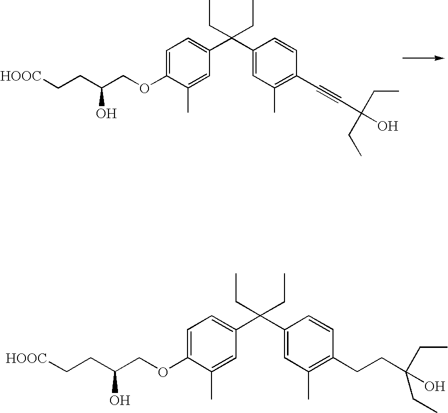

To a solution of (S)-5-(4-{1-ethyl-1-[4-(3-ethyl-3-hydroxy-pent-1-ynyl)-3-methyl-phenyl]-propyl}-2-methyl-phenoxy)-4-hydroxy-pentanoic acid (compound prepared in Example 23-(2)) (50 mg, 0.101 mmol) in EtOH (10 ml) was added Pd(OH)₂ (10 mg) and the mixture was stirred under hydrogen gas at room temperature for 12 h. The reaction mixture was filtered through celite. and concentrated in vacuo. The crude mixture (40 mg) was dissolved in MeOH (7 ml). 1N-KOH (0.3 ml, 0.30 mmol) was added at room temperature and the mixture was stirred for 4 h. The reaction mixture was concentration in vacuo, diluted with EtOAc, washed with brine, dried over MgSO₄, filtered, and concentration in vacuo. The obtained residue was purified by silica gel chromatography eluting with CH₂Cl₂/EtOAc (20:1) to give the title compound (26 mg, 52%) as a white solid.

¹H-NMR (300 MHz, CDCl₃): 0.59 (t, 6H), 0.91 (t, 6H), 1.51-1.59 (m, 4H), 1.63-1.69 (m, 2H), 1.88-1.96 (m, 2H), 2.00-2.07 (q, 4H), 2.17 (s,3H), 2.25 (s,3H), 2.54-2.65 (m, 4H), 3.82-3.87 (dd, 1H), 3.95-3.99 (dd, 1H), 4.05-4.11 (m, 1H), 6.67 (d, 1H), 6.86 (d, 1H), 6.89-7.01 (m, 5H); MS (ESI−): 497 ([M−H]⁻).

### Example 25

### Preparation of (S)-5-(4-{1-ethyl-1-[4-(3-hydroxy-3-propyl-hex-1-ynyl)-3-methyl-phenyl]-propyl}-2-methyl-phenoxy)-4-hydroxy-pentanoic acid

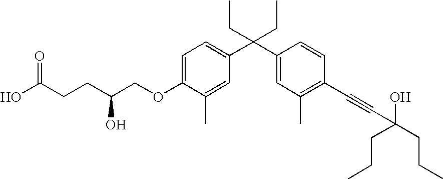

### (1) Preparation of (S)-5-(4-{1-ethyl-1-[4-(3-hydroxy-3-propyl-hex-1-ynyl)-3-methyl-phenyl]-propyl}-2-methyl-phenoxymethyl)-dihydro-furan-2-one

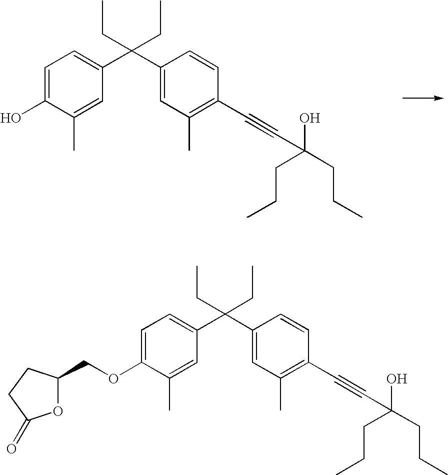

Using the same procedure as described for the preparation of Example 1-(6), the title compound was prepared from 4-{1-ethyl-1-[4-(3-hydroxy-3-propyl-hex-1-ynyl)-3-methyl-phenyl]-propyl}-2-methyl-phenol (compound prepared in Example 4). The yield was 78%.

¹H-NMR (300 MHz, CDCl₃): 0.59 (t, 6H), 0.98 (t, 6H), 1.54-1.74 (m, 8H), 2.02 (q, 4H), 2.14 (s, 3H), 2.26 (m, 1H), 2.36 (s, 3H), 2.39-2.62 (m, 3H), 2.71-2.82 (m, 1H), 4.06 (dd, 1H), 4.14 (dd, 1H), 4.87 (m, 1H), 6.65 (d, 1H), 6.87 (s, 1H), 6.95 (dd, 2H), 6.98 (s, 1H), 7.27 (d, 1H).

### (2) Preparation of (S)-5-(4-{1-ethyl-1-[4-(3-hydroxy-3-propyl-hex-1-ynyl)-3-methyl-phenyl]-propyl}-2-methyl-phenoxy)-4-hydroxy-pentanoic acid

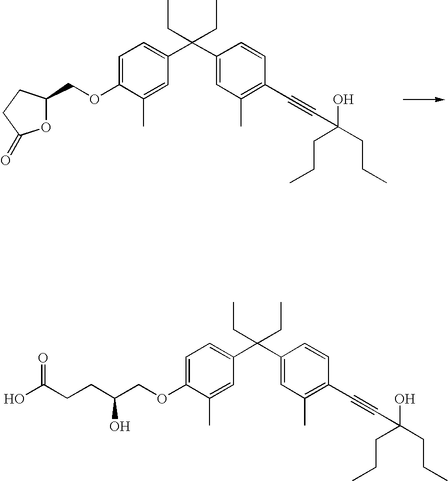

Using the same procedure as described for the preparation of Example 1-(7), the title compound was prepared from (S)-5-(4-{1-ethyl-1-[4-(3-hydroxy-3-propyl-hex-1-ynyl)-3-methyl-phenyl]-propyl}-2-methyl-phenoxymethyl)-dihydro-furan-2-one (compound prepared in Example 25-(1)). The yield was 95%.

¹H-NMR (300 MHz, CD₃OD as potassium salt): 0.59 (t, 6H), 1.00 (t, 6H), 1.52-1.92 (m, 10H), 2.06 (q, 4H), 2.16 (s, 3H), 2.35 (s,3H), 2.38 (td, 2H), 3.91-3.98 (m, 3H), 6.78 (d, 1H), 6.85 (s, 1H), 6.96 (d, 2H), 7.01 (s, 1H), 7.23 (d, 1H); MS (ESI−): 521 ([M−H]⁻).

### Example 26

### Preparation of (S)-5-(4-{1-ethyl-1-[4-((E)-3-hydroxy-3-propyl-hex-1-enyl)-3-methyl-phenyl]-propyl}-2-methyl-phenoxy)-4-hydroxy-pentanoic acid

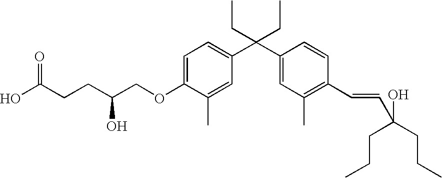

### (1) Preparation of (S)-5-(4-{1-ethyl-1-[4-((E)-3-hydroxy-3-propyl-hex-1-enyl)-3-methyl-phenyl]-propyl}-2-methyl-phenoxymethyl)-dihydro-furan-2-one

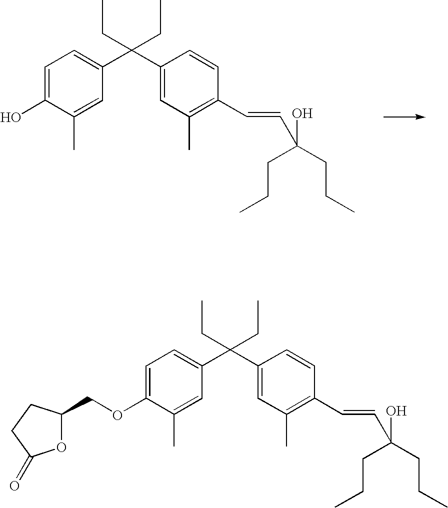

Using the same procedure as described for the preparation of Example 1-(6), the title compound was prepared from 4-{1-ethyl-1-[4-((E)-3-hydroxy-3-propyl-hex-1-enyl)-3-methyl-phenyl]-propyl}-2-methyl-phenol (compound prepared in Example 13). The yield was 70%.

¹H-NMR (300 MHz, CDCl₃): 0.59 (t, 6H), 0.98 (t, 6H), 1.54-1.74 (m, 8H), 2.02 (q, 4H), 2.14 (s, 3H), 2.26 (m, 1H), 2.36 (s, 3H), 2.39-2.62 (m, 3H), 2.71-2.82 (m, 1H), 4.06 (dd, 1H), 4.14 (dd, 1H), 4.87 (m, 1H), 6.07 (d, 1H), 6.75 (dd, 1H), 6.87 (d, 2H), 6.94 (s, 2H), 6.99 (td, 1H), 7.29 (d, 1H).

### (2) Preparation of (S)-5-(4-{1-ethyl-1-[4-((E)-3-hydroxy-3-propyl-hex-1-enyl)-3-methyl-phenyl]-propyl}-2-methyl-phenoxy)-4-hydroxy-pentanoic acid

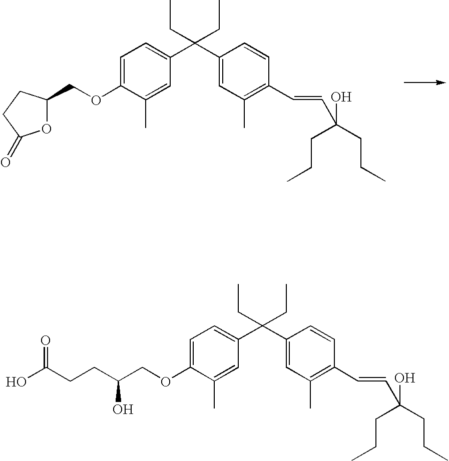

Using the same procedure as described for the preparation of Example 1-(7), the title compound was prepared from (S)-5-(4-{1-ethyl-1-[4-((E)-3-hydroxy-3-propyl-hex-1-enyl)-3-methyl-phenyl]-propyl}-2-methyl-phenoxymethyl)-dihydro-furan-2-one (compound prepared in Example 26-(1)). The yield was 93%.

¹H-NMR (300 MHz, CD₃OD as potassium salt): 0.62 (t, 6H), 0.95 (t, 6H), 1.39-1.51 (m, 4H), 1.56-1.62 (m, 4H), 1.87-2.02 (m,2H), 2.07 (q, 4H), 2.17 (s, 3H), 2.29 (s,3H), 2.39 (td, 2H), 3.93 (d, 2H), 3.98 (m, 1 H), 6.07 (d, 1H), 6.75 (dd, 1H), 6.87 (d, 2H), 6.94 (s, 2H), 6.99 (td, 1H), 7.29 (d, 1H); MS (ESI−): 523 ([M−H]⁻).

### Example 27

### Preparation of 5-(4-{1-ethyl-1-[4-(3-hydroxy-4,4-dimethyl-pent-1-ynyl)-3-methyl-phenyl]-propyl}-2-methyl-phenoxy)-4(S)-hydroxy-pentanoic acid

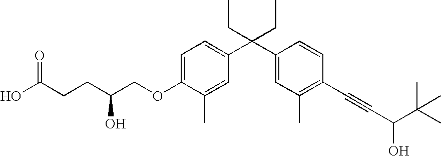

### (1) Preparation of (S)-5-(4-{1-ethyl-1-[4-(3-hydroxy-4,4-dimethyl-pent-1-ynyl)-3-methyl-phenyl]-propyl}-2-methyl-phenoxymethyl)-dihydro-furan-2-one

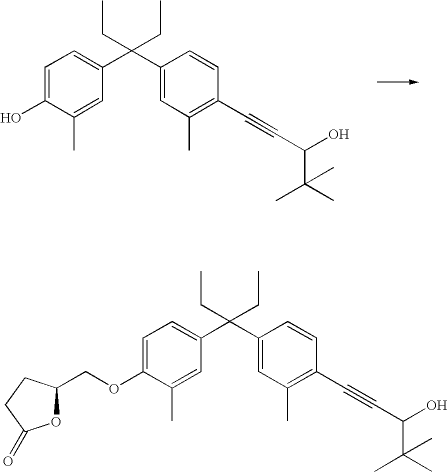

Using the same procedure as described for the preparation of Example 1-(6), the title compound was prepared from 4-{1-ethyl-1-[4-(3-hydroxy-4,4-dimethyl-pent-1-ynyl)-3-methyl-phenyl]-propyl}-2-methyl-phenol (compound prepared in Example 3). The yield was 38%.

¹H-NMR (300 MHz, CDCl₃): 0.58 (t, 6H), 1.06 (s, 9H), 1.76 (d, 1H), 2.04 (q, 4H), 2.14 (s, 3H), 2.38 (s, 3H), 2.25-2.62 (m, 3H), 2.71-2.83 (m, 1H), 4.06 (dd, 1H), 4.16 (dd, 1H), 4.25 (d, 1H), 4.84-4.91 (m, 1H), 6.66 (d, 1H), 6.87-6.98 (m, 4H), 7.28 (d, 1H); MS (ESI+): 499 ([M+Na]⁺).

### (2) Preparation of 5-(4-{1-ethyl-1-[4-(3-hydroxy-4,4-dimethyl-pent-1-ynyl)-3-methyl-phenyl]-propyl}-2-methyl-phenoxy)-4(S)-hydroxy-pentanoic acid

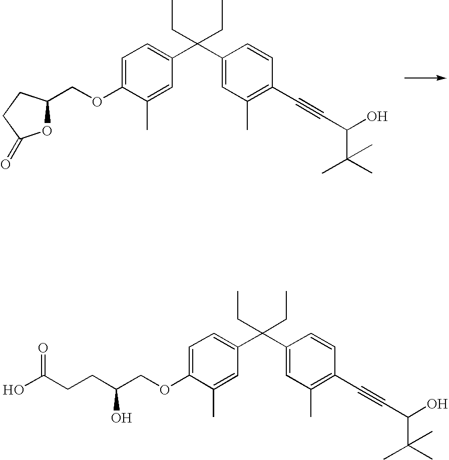

Using the same procedure as described for the preparation of Example 1-(7), the title compound was prepared from (S)-5-(4-{1-ethyl-1-[4-(3-hydroxy-4,4-dimethyl-pent-1-ynyl)-3-methyl-phenyl]-propyl}-2-methyl-phenoxymethyl)-dihydro-furan-2-one (compound prepared in Example 27-(1)). The yield was 43%.

¹H-NMR (300 MHz, CD₃OD as potassium salt): 0.62 (t, 6H), 1.08 (s, 9H), 1.80-2.03 (m, 2H), 2.10 (q, 4H), 2.18 (s, 3H), 2.38-2.42 (m, 5H), 3.92-4.03 (m, 3H), 4.22 (s, 1H), 6.80 (d, 1H), 6.87 (1H, m), 6.95-7.03 (m, 3H), 7.28 (d, 1H); MS (ESI+): 517 ([M+Na]⁺).

### Example 28

### Preparation of 5-(4-{1-ethyl-1-[4-((E)-3-hydroxy-4,4-dimethyl-pent-1-enyl)-3-methyl-phenyl]-propyl}-2-methyl-phenoxy)-4(S)-hydroxy-pentanoic acid

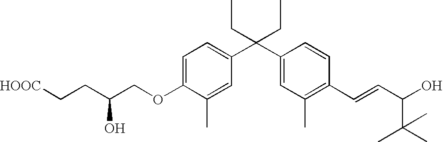

### (1) Preparation of (S)-5-(4-{1-ethyl-1-[4-((E)-3-hydroxy-4,4-dimethyl-pent-1-enyl)-3-methyl-phenyl]-propyl}-2-methyl-phenoxymethyl)-dihydro-furan-2-one

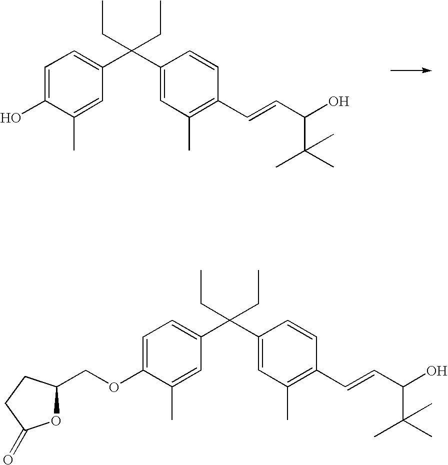

Using the same procedure as described for the preparation of Example 1-(6), the title compound was prepared from 4-{1-ethyl-1-[4-((E)-3-hydroxy-4,4-dimethyl-pent-1-enyl)-3-methyl-phenyl]-propyl}-2-methyl-phenol (compound prepared in Example 14). The yield was 78%.

¹H-NMR (CDCl₃): 0.60 (t, 6H), 0.96 (s, 9H), 2.04 (q, 4H), 2.15 (s, 3H), 2.29 (s, 3H), 2.30-2.61 (m, 3H), 2.70-2.82 (m, 1H), 2.92 (d, 1H), 4.03-4.18 (m, 2H), 4.83-4.90 (m, 1H), 6.12 (dd, 1H), 6.66 (d, 1H), 6.91-6.97 (m, 5H), 7.30 (d, 1H); MS (ESI+): 496 ([M+NH₄]⁺).

### (2) Preparation of 5-(4-{1-ethyl-1-[4-((E)-3-hydroxy-4,4-dimethyl-pent-1-enyl)-3-methyl-phenyl]-propyl}-2-methyl-phenoxy)-4(S)-hydroxy-pentanoic acid

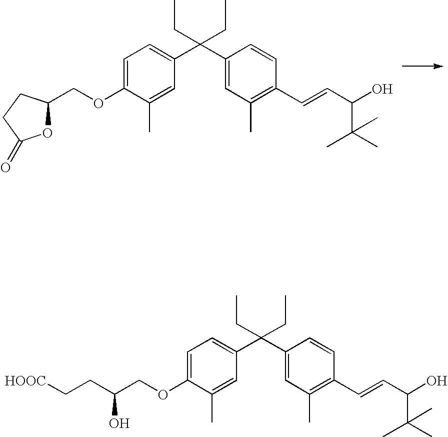

Using the same procedure as described for the preparation of Example 1-(7), the title compound was prepared from (S)-5-(4-{1-ethyl-1-[4-((E)-3-hydroxy-4,4-dimethyl-pent-1-enyl)-3-methyl-phenyl]-propyl}-2-methyl-phenoxymethyl)-dihydro-furan-2-one (compound prepared in Example 28-(1)). The yield was 41%.

¹H-NMR (300 MHz, CD₃OD as potassium salt): 0.60 (t, 6H), 0.96 (s, 9H), 1.88-1.97 (m, 2H), 2.05 (q, 4H), 2.17 (s, 3H), 2.29 (s, 3H), 2.62 (t, 2H), 3.82-3.99 (m, 2H), 4.03-4.11 (m, 1H), 4.13-4.18 (m, 1H), 4.42-4.49 (m, 1H), 6.12 (dd, 1H), 6.66 (d,1H), 6.73 (d, 1H), 6.90-6.96 (m, 4H), 7.30 (d, 1H); MS (ESI−): 495 ([M−H]⁻).

### Example 29

### Preparation of 5-(4-{1-ethyl-1-[4-(3-hydroxy-4,4-dimethyl-pentyl)-3-methyl-phenyl]-propyl}-2-methyl-phenoxy)-4(S)-hydroxy-pentanoic acid

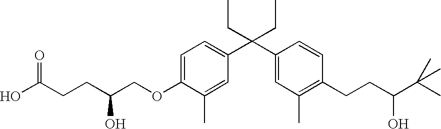

### (1) Preparation of (S)-5-(4-{1-ethyl-1-[4-(3-hydroxy-4,4-dimethyl-pentyl)-3-methyl-phenyl]-propyl}-2-methyl-phenoxymethyl)-dihydro-furan-2-one

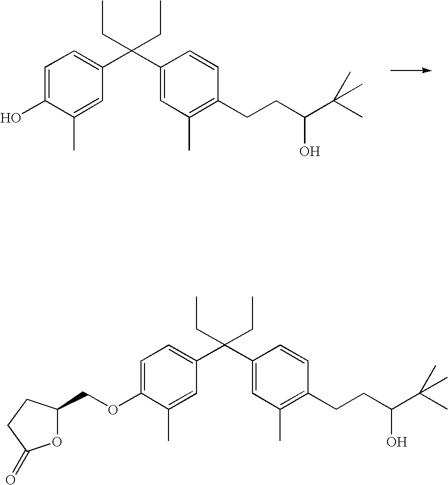

Using the same procedure as described for the preparation of Example 1-(6), the title compound was prepared from 4-{1-ethyl-1-[4-(3-hydroxy-4,4-dimethyl-pentyl)-3-methyl-phenyl]-propyl}-2-methyl-phenol (compound prepared in Example 18). The yield was 88%.

¹H-NMR (300 MHz, CD₃OD): 0.59 (t, 6H), 0.88 (s, 9H), 1.38-1.52 (m, 1H), 1.72-2.02 (m, 3H), 2.09 (q, 4H), 2.15 (s, 3H), 2.25 (s,3H), 2.42 (td, 2H), 2.48-2.59 (m, 1H), 2.87 (m, 1H), 3.18 (dd, 1H), 3.90-3.99 (m, 3H), 4.02-4.19 (m, 2H), 4.90 (m, 1H), 6.75 (d, 1H), 6.86-6.98(m, 4H), 7.02 (d, 1H).

### (2) Preparation of 5-(4-{1-ethyl-1-[4-(3-hydroxy-4,4-dimethyl-pentyl)-3-methyl-phenyl]-propyl}-2-methyl-phenoxy)-4(S)-hydroxy-pentanoic acid

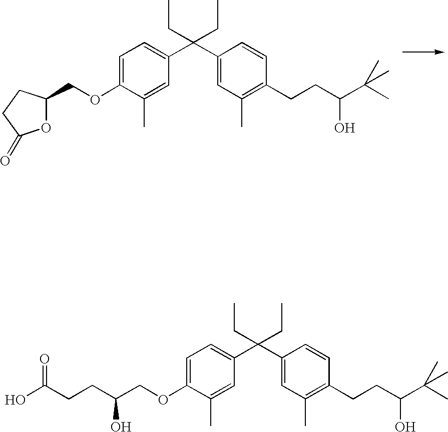

Using the same procedure as described for the preparation of Example 1-(7), the title compound was prepared from (S)-5-(4-{1-ethyl-1-[4-(3-hydroxy-4,4-dimethyl-pentyl)-3-methyl-phenyl]-propyl}-2-methyl-phenoxymethyl)-dihydro-furan-2-one (compound prepared in Example 29-(1)). The yield was 76%.

¹H-NMR (300 MHz, CD₃OD as potassium salt): 0.59 (t, 6H), 0.88 (s, 9H), 1.56 (m, 1H), 1.72-2.05 (m, 3H), 2.09 (q, 4H), 2.15 (s, 3H), 2.25 (s,3H), 2.37-2.43 (td, 2H), 2.54-2.57 (m, 1H), 2.83-2.89 (m, 1H), 3.20 (dd, 1H), 3.90-3.97 (m, 3H), 6.75 (d, 1H), 6.86-6.97 (m, 4H), 7.02 (d, 1H); MS (ESI−): 497 ([M−H]⁻).

### Example 30

### (S)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid

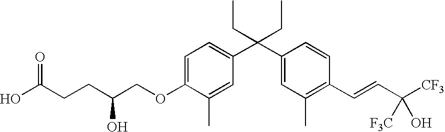

### (1) Preparation of 4-{1-ethyl-1-[3-methyl-4-((E)-4,4,4-trifluoro-3-methoxymethoxy-3-trifluoromethyl-but-1-enyl)-phenyl]-propyl}-2-methyl-phenol

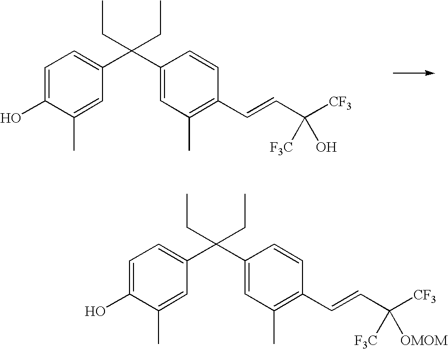

To a solution of 4-{1-ethyl-1-[3-methyl-4-((E)-4,4,4-trifluoro-3-hydroxy-3-trifluoromethyl-but-1-enyl)-phenyl]-propyl}-2-methyl-phenol (compound prepared in Example 9) (400 mg, 0.87 mmol) in DMF (5 ml) was added NaH (34.8 mg, 0.87 mmol) and the mixture was stirred at 25 degrees C. for 0.5 h. To the reaction mixture, methoxymethyl chloride (0.132 ml, 1.74 mmol) was added and stirred at 50 degrees C. for overnight. The mixture was cooled to 0 degrees C., saturated NH₄Cl was added and extracted with diethyl ether. The organic layer was washed with brine, dried over Na₂SO₄, concentrated in vacuo, and chromatographed on silica gel (AcOEt/hexane/dichloromethane=1/1/10) to give the title compound (180 mg, 41%).

¹H NMR (CDCl₃): 7.36 (s, 1H), 7.33 (d, 1H), 7.00 (d, 1H), 6.99 (s, 1H), 6.91-6.84 (m, 2H), 6.65 (d, 1H), 6.07 (d, 1H), 4.96 (s, 2H), 4.61 (s, 1H), 3.50 (s, 3H), 2.82-2.24 (m, 4H), 2.32 (s, 3H), 2.19 (s, 3H), 2.09 (q, 4H), 0.60 (t, 6H).

### (2) Preparation of (S)-5-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-dihydro-furan-2-one

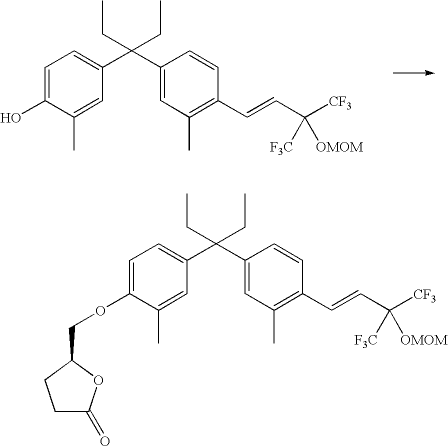

Using the same procedure as described for the preparation of Example 1-(6), the title compound was prepared from 4-{1-ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in Example 30-(1)). The yield was 62%.

¹H-NMR (CDCl₃): 7.36 (s,1H), 7.33 (d, 1H), 7.01-6.90 (m, 4H), 6.67 (d, 1 H), 6.05 (d, 1H), 4.96 (s, 2H), 4.90-4.84 (m, 1H), 4.19˜4.04 (m, 2H), 3.50 (s, 3H), 2.82-2.24 (m, 4H), 2.32 (s, 3H), 2.19 (s, 3H), 2.04 (q, 4H), 0.60 (t, 6H).

### (3) Preparation of (S)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid

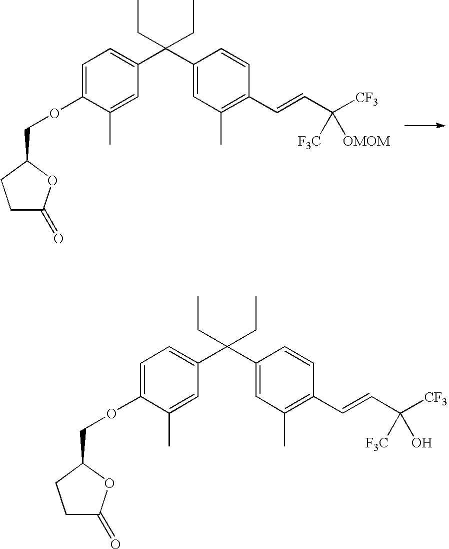

To a solution of (S)-5-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-dihydro-furan-2-one (compound prepared in Example 30-(2)) (134 mg, 0.22 mmol) in 1,4-dioxane(5 ml) was added conc-HCl (0.5 ml) and the mixture was stirred at room temperature for 13 h. The reaction mixture was poured into H₂O and extracted with ethyl acetate. The organic layer was washed with brine, dried over Na₂SO₄, concentrated in vacuo, and the obtained residue was chromatographed on silica gel (AcOEt/hexane/dichloromethane=1/1/10) to give the title compound (85 mg, 69%).

¹H-NMR (CDCl₃): 7.39-7.26 (m, 2H), 7.01-6.90 (m, 4H), 6.67 (d, 1H), 6.07 (d, 1H), 4.90-4.84 (m, 1H), 4.19-4.04 (m, 2H), 3.22 (s, 1H), 2.82-2.24 (m, 4H), 2.32 (s, 3H), 2.19 (s, 3H), 2.04 (q, 4H), 0.60 (t, 6H).

### (4) Preparation of (S)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid

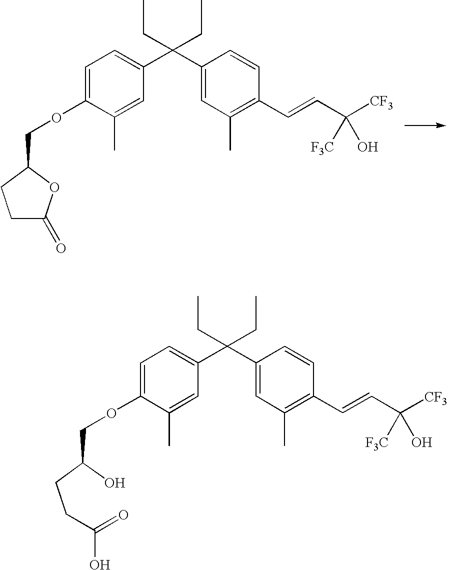

Using the same procedure as described for the preparation of Example 1-(7), the title compound was prepared from (S)-5-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-dihydro-furan-2-one (compound prepared in Example 35-(3)). The yield was 49%.

¹H-NMR (CD₃OD as potassium salt): 7.43 (d, 1H), 7.34 (d, 1H), 6.99-6.87 (m, 4H), 6.69 (d, 1H), 6.23 (d, 1H), 4.00-3.91 (m, 3H), 2.41-1.81 (m, 14H), 0.60 (t, 6H).; MS (ESI−):575 ([M−H]⁻).

### Example 31

### Preparation of (S)-5-(4-{1-ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid

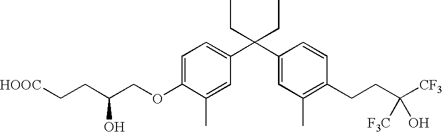

### (1) Preparation of (S)-5-(4-{1-ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one

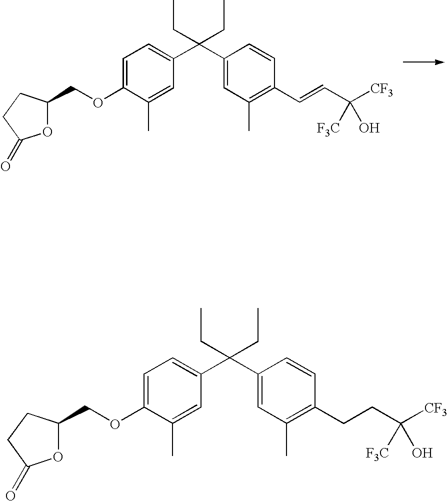

To a solution of (S)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one (compound prepared in Example 30-(3)) (45 mg, 0.08 mmol) in MeOH (5 ml) was added palladium hydroxide (4.5 mg 10% w/w)) at room temperature under H₂ gas and the mixture was stirred for 3 h. The reaction mixture was filtered through celite and concentrated under reduced pressure. The obtained residue was purified by silica gel chromatography (Hexane/CH₂Cl₂/EtOAc=1:1:1)to give the title compound (45 mg, quant).

¹H-NMR (CDCl₃); 7.01-6.80 (m, 5H), 6.70 (d, 1H), 4.90-4.84 (m, 1H), 4.19-4.04 (m, 2H), 2.88-1.90 (m, 18H), 0.60 (t, 6H).

### (2) Preparation of (S)-5-(4-{1-ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid

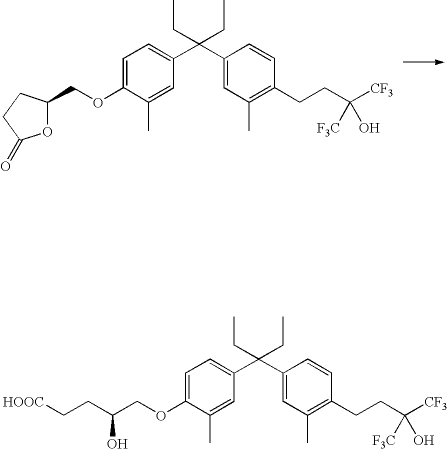

Using the same procedure as described for the preparation of Example 1-(7), the title compound was prepared from (S)-5-(4-{1-ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one (compound prepared in Example 31-(1)). The yield was 54%.

¹H-NMR (300 MHz, CD₃OD as potassium salt): 7.05-6.88 (m, 5H), 6.80 (d, 1H), 4.01-3.92 (m, 3H), 2.87-2.77 (m, 4H), 2.44-1.81 (m, 14H), 0.60 (t, 6H); MS (ESI−): 577 ([M−H]⁻).

### Example 32

### Preparation of (S)-5-(4-{1-ethyl-1-[4-(1-hydroxy-cyclobutylethynyl)-3-methyl-phenyl]-propyl}-2-methyl-phenoxy)-4-hydroxy-pentanoic acid

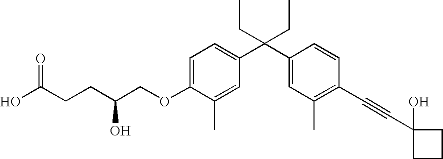

### (1) Preparation of (S)-5-(4-{1-ethyl-1-[4-(1-hydroxy-cyclobutylethynyl)-3-methyl-phenyl]-propyl}-2-methyl-phenoxymethyl)-dihydro-furan-2-one

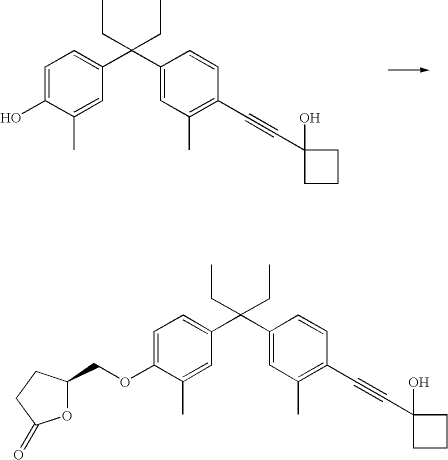

Using the same procedure as described for the preparation of Example 1-(6), the title compound was prepared from 4-{1-ethyl-1-[4-(1-hydroxy-cyclobutylethynyl)-3-methyl-phenyl]-propyl}-2-methyl-phenol (compound prepared in Example 6). The yield was 51%.

¹H-NMR (300 MHz, CDCl₃): 0.58 (t, 6H), 1.64-1.90 (m, 4H), 2.02 (q, 4H), 2.14 (s, 3H), 2.26 (m, 2H), 2.34 (s, 3H), 2.39-2.62 (m, 3H), 2.71-2.82 (m, 1H), 4.06 (dd, 1H), 4.14 (dd, 1H), 4.87 (m, 1H), 6.68 (d, 1H), 6.84 (m, 2H), 6.91 (d, 1H), 7.00 (s, 1H), 7.27 (d, 1H).

### (2) Preparation of (S )-5-(4-{1-ethyl-1-[4-(1-hydroxy-cyclobutylethynyl)-3-methyl-phenyl]-propyl}-2-methyl-phenoxy)-4-hydroxy-pentanoic acid

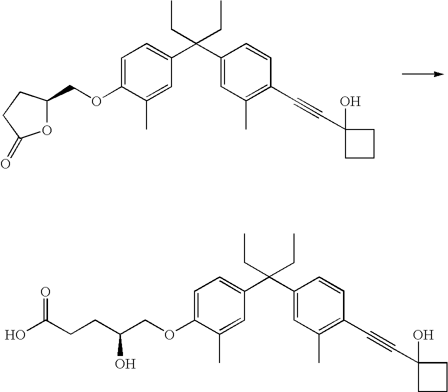

Using the same procedure as described for the preparation of Example 1-(7), the title compound was prepared from (S)-5-(4-{1-ethyl-1-[4-(1-hydroxy-cyclobutylethynyl)-3-methyl-phenyl]-propyl}-2-methyl-phenoxymethyl)-dihydro-furan-2-one (compound prepared in Example 32-(1)). The yield was 95%.

¹H-NMR (300 MHz, CD₃OD as potassium salt): 0.62 (t, 6H), 1.79-2.01 (m,4H), 2.06 (q, 4H), 2.20 (s,3H), 2.25 (m, 4H), 2.32 (s, 3H), 2.39 (td, 2H), 3.89-3.98 (m, 3H), 6.73 (d,1 H), 6.80 (m, 2H), 6.91 (d, 2H), 6.96 (s, 1H), 7.20 (d, 1H); MS (ESI−): 477 ([M−H]⁻).

### Example 33

### Preparation of (S)-5-[4-(1-ethyl-1-{4-[(E)-2-(1-hydroxy-cyclobutyl)-vinyl]-3-methyl-phenyl}-propyl)-2-methyl-phenoxy]-4-hydroxy-pentanoic acid

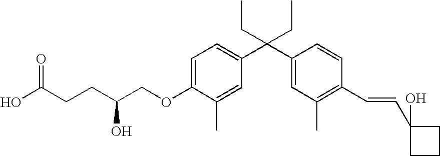

### (1) Preparation of (S)-5-[4-(1-ethyl-1-{4-[(E)-2-(1-hydroxy-cyclobutyl)-vinyl]-3-methyl-phenyl}-propyl)-2-methyl-phenoxymethyl]-dihydro-furan-2-one

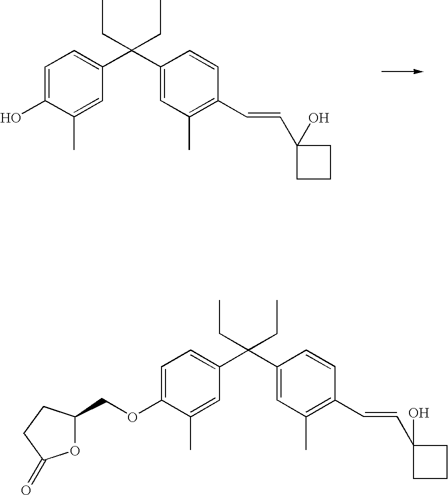

Using the same procedure as described for the preparation of Example 1-(6), the title compound was prepared from 4-(1-ethyl-1-{4-[(E)-2-(1-hydroxy-cyclobutyl)-vinyl]-3-methyl-phenyl}-propyl)-2-methyl-phenol (compound prepared in Example 15). The yield was 74%.

¹H-NMR (300 MHz, CDCl₃): 0.60 (t, 6H), 1.64-1.90 (m, 4H), 2.04 (q, 4H), 2.15 (s, 3H), 2.26 (m, 2H), 2.34 (s, 3H), 2.39-2.62 (m, 3H), 2.71-2.82 (m, 1H), 4.06 (dd, 1H), 4.14 (dd, 1H), 4.84-4.91 (m, 1H), 6.34 (d, 1H), 6.66 (d, 1H), 6.81 (d, 1H), 6.91-6.97 (m, 4H), 7.40 (d, 1H).

### (2) Preparation of (S)-5-[4-(1-ethyl-1-{4-[(E)-2-(1-hydroxy-cyclobutyl)-vinyl]-3-methyl-phenyl}-propyl)-2-methyl-phenoxy]-4-hydroxy-pentanoic acid

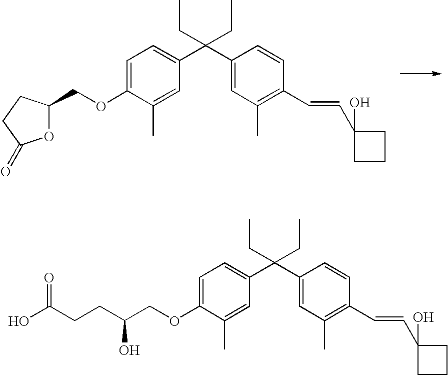

Using the same procedure as described for the preparation of Example 1-(7), the title compound was prepared from (S)-5-[4-(1-ethyl-1-{4-[(E)-2-(1-hydroxy-cyclobutyl)-vinyl]-3-methyl-phenyl}-propyl)-2-methyl-phenoxymethyl]-dihydro-furan-2-one (compound prepared in Example 33-(1)). The yield was 98%.

¹H-NMR (300 MHz, CD₃OD as potassium salt): 0.59 (t, 6H), 1.52-1.82 (m, 4H), 1.83-1.92 (m, 1H), 1.87-2.10 (m,2H), 1.97 (q, 4H), 2.06 (s, 3H), 2.19 (s, 3H), 2.15 (m, 4 H), 2.31 (td, 2H), 3.81-3.89 (m, 3H), 6.25 (d, 1H), 6.67-6.77 (m, 3H), 6.84-6.88 (m, 3H), 7.25 (d, 1H); MS (ESI−): 479 ([M−H]⁻).

### Example 34

### Preparation of (S)-5-(4-{1-ethyl-1-[4-(1-hydroxy-cyclopentylethynyl)-3-methyl-phenyl]-propyl}-2-methyl-phenoxy)-4-hydroxy-pentanoic acid

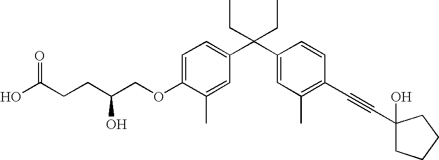

### (1) Preparation of (S)-5-(4-(1-ethyl-1-[4-(1-hydroxy-cyclopentylethynyl)-3-methyl-phenyl]-propyl}-2-methyl-phenoxymethyl)-dihydro-furan-2-one

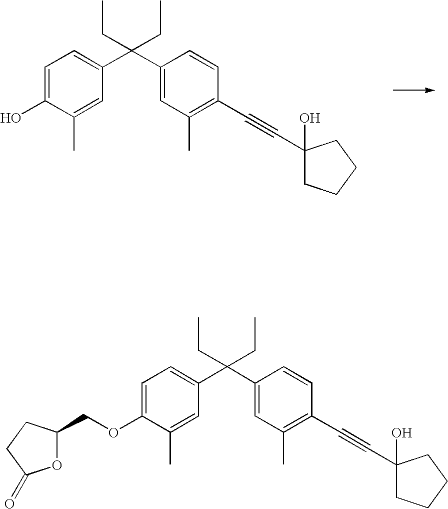

Using the same procedure as described for the preparation of Example 1-(6), the title compound was prepared from 4-{1-ethyl-1-[4-(1-hydroxy-cyclopentylethynyl)-3-methyl-phenyl]-propyl}-2-methyl-phenol (compound prepared in Example 7). The yield was 58%.

¹H-NMR (300 MHz, CDCl₃): 0.58 (t, 6H), 1.75-1.95 (m, 5H), 1.97-2.10 (m, 8H), 2.14 (s, 3H), 2.27-2.35 (m, 1H), 2.36 (s, 3H), 2.39-2.62 (m, 2H), 2.71-2.82 (m, 1H), 4.06 (dd, 1H), 4.14 (dd, 1H), 4.84-4.91 (m, 1H), 6.66 (d, 1H), 6.85 (s, 1H), 6.94 (d, 2H), 6.90 (s, 1H), 7.26 (d, 1H); MS(ESI+): 497 ([M+Na]⁺).

### (2) Preparation of (S)-5-(4-{1-ethyl-1-[4-(1-hydroxy-cyclopentylethynyl)-3-methyl-phenyl]-propyl}-2-methyl-phenoxy)-4-hydroxy-pentanoic acid

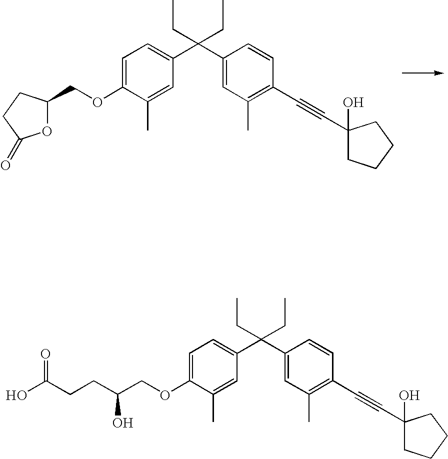

Using the same procedure as described for the preparation of Example 1-(7), the title compound was prepared from (S)-5-(4-{1-ethyl-1-[4-(1-hydroxy-cyclopentylethynyl)-3-methyl-phenyl]-propyl}-2-methyl-phenoxymethyl)-dihydro-furan-2-one (compound prepared in Example 34-(1)). The yield was 83%.

¹H-NMR (300 MHz, CD₃OD as potassium salt): 0.60 (t, 6H), 1.81-1.85 (m, 5H), 1.91-2.01 (m, 5H), 2.02 (q, 4H), 2.16 (s, 3H), 2.34 (s,3H), 2.39 (td, 2H), 3.90-3.99 (m, 3H), 6.78 (d, 1H), 6.84 (s, 1H), 6.94 (dd, 2H), 7.00 (s, 1H), 7.22 (d, 1H); MS (ESI−): 491 ([M−H]⁻).

### Example 35

### Preparation of (S)-5-[4-(1-ethyl-1-{4-[(E)-2-(1-hydroxy-cyclopentyl)-vinyl]-3-methyl-phenyl}-propyl)-2-methyl-phenoxy]-4-hydroxy-pentanoic acid

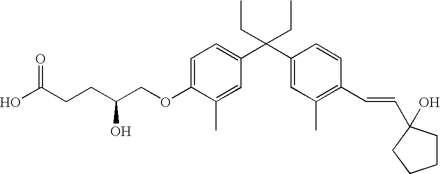

### (1) Preparation of (S)-5-[4-(1-ethyl-1-{4-[(E)-2-(1-hydroxy-cyclopentyl)-vinyl]-3-methyl-phenyl}-propyl)-2-methyl-phenoxymethyl]-dihydro-furan-2-one

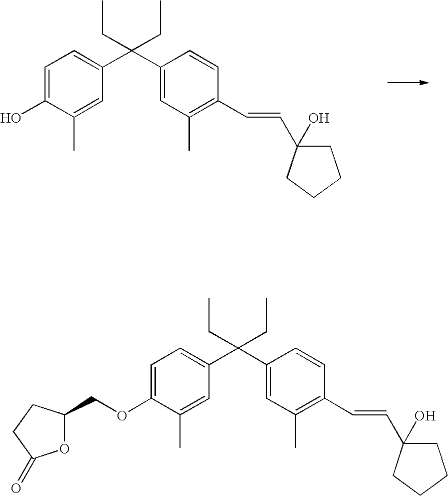

Using the same procedure as described for the preparation of Example 1-(6), the title compound was prepared from 4-(1-ethyl-1-{4-[(E)-2-(1-hydroxy-cyclopentyl)-vinyl]-3-methyl-phenyl}-propyl)-2-methyl-phenol (compound prepared in Example 10). The yield was 54%.

¹H-NMR (300 MHz, CDCl₃): 0.58 (t, 6H), 1.75-1.95 (m, 5H), 1.97-2.10 (m, 8H), 2.14 (s, 3H), 2.34 (s,3H), 2.38 (m, 1H), 2.39-2.62 (m, 2H), 2.71-2.82 (m, 1H), 4.06 (dd, 1H), 4.14 (dd, 1H), 4.84-4.91 (m, 1H), 6.23 (d, 1H), 6.68 (d, 1H), 6.81 (d, 1H), 6.90-6.99 (m, 4H), 7.32 (d, 1H); MS (ESI+): 499 ([M+Na]⁺).

### (2) Preparation of (S)-5-[4-(1-ethyl-1-{4-[(E)-2-(1-hydroxy-cyclopentyl)-vinyl]-3-methyl-phenyl}-propyl)-2-methyl-phenoxy]-4-hydroxy-pentanoic acid

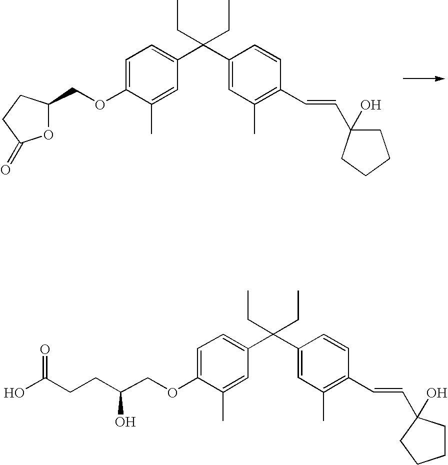

Using the same procedure as described for the preparation of Example 1-(7), the title compound was prepared from (S)-5-[4-(1-ethyl-1-{4-[(E)-2-(1-hydroxy-cyclopentyl )-vinyl]-3-methyl-phenyl}-propyl)-2-methyl-phenoxymethyl]-dihydro-furan-2-one (compound prepared in Example 35-(1)). The yield was 97%.

¹H-NMR (300 MHz, CD₃OD as potassium salt): 0.60 (t, 6H), 1.76-2.00 (m, 12H), 2.06 (q, 4H), 2.16 (s, 3H), 2.29 (s,3H), 2.41 (td, 2H), 3.90-3.99 (m, 3H), 6.23 (d, 1H), 6.77 (d,1H), 6.84 (d, 1H), 6.93-6.99 (m, 4H), 7.32 (d,1H); MS (ESI−): 493 ([M−H]⁻).

### Example 36

### Preparation of (S)-5-(4-{1-ethyl-1-[4-(1-hydroxy-cyclohexylethynyl)-3-methyl-phenyl]-propyl}-2-methyl-phenoxy)-4-hydroxy-pentanoic acid

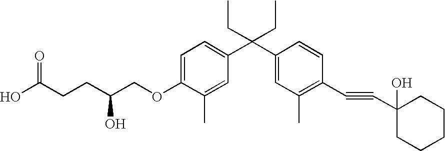

### (1) Preparation of (S)-5-(4-{1-ethyl-1-[4-(1-hydroxy-cyclohexylethynyl)-3-methyl-phenyl]-propyl}-2-methyl-phenoxymethyl)-dihydro-furan-2-one

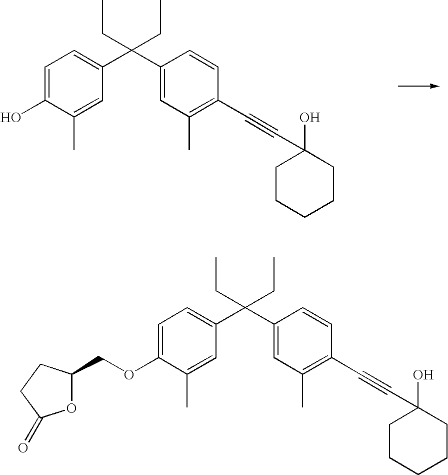

Using the same procedure as described for the preparation of Example 1-(6), the title compound was prepared from 4-{1-ethyl-1-[4-(1-hydroxy-cyclohexylethynyl)-3-methyl-phenyl]-propyl}-2-methyl-phenol (compound prepared in Example 8). The yield was 72%.

¹H-NMR (300 MHz, CDCl₃): 0.59 (t, 6H), 1.61-1.79 (m, 6H), 1.99-2.09 (m, 8H), 2.14 (s, 3H), 2.26 (m, 1H), 2.38 (s, 3H), 2.39-2.62 (m, 3H), 2.71-2.82 (m, 1H), 4.08 (dd, 1H), 4.14 (dd, 1H), 4.84-4.91 (m, 1H), 6.65 (d, 1H), 6.87 (s, 1H), 6.92 (d, 2H), 7.00 (s, 1H), 7.27 (d, 1H).

### (2) Preparation of (S)-5-(4-{1-ethyl-1-[4-(1-hydroxy-cyclohexylethynyl)-3-methyl-phenyl]-propyl}-2-methyl-phenoxy)-4-hydroxy-pentanoic acid

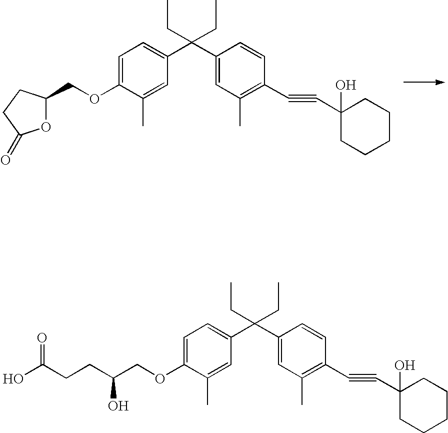

Using the same procedure as described for the preparation of Example 1-(7), the title compound was prepared from (S)-5-(4-{1-ethyl-1-[4-(1-hydroxy-cyclohexylethynyl)-3-methyl-phenyl]-propyl)-2-methyl-phenoxymethyl)-dihydro-furan-2-one (compound prepared in Example 36-(1)). The yield was 92%.

¹H-NMR (300 MHz, CD₃OD as potassium salt): 0.59 (t, 6H), 1.59-1.89 (m, 9H), 1.95-2.03 (m, 3H), 2.08 (q, 4H), 2.15 (s, 3H), 2.35 (s,3H), 2.36 (td, 2H), 3.89- 3.98 (m, 3H), 6.77 (d, 1 H), 6.84 (s, 1 H), 6.95 (d, 1 H), 7.00 (s, 1 H), 7.23 (d, 1H); MS (ESI−): 505 ([M−H]⁻).

### Example 37

### Preparation of (S)-5-[4-(1-ethyl-1-{4-[(E)-2-(1-hydroxy-cyclohexyl)-vinyl]-3-methyl-phenyl}-propyl)-2-methyl-phenoxy]-4-hydroxy-pentanoic acid

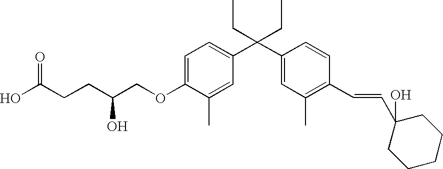

### (1) Preparation of (S)-5-[4-(1-ethyl-1-{4-[(E)-2-(1-hydroxy-cyclohexyl)-vinyl]-3-methyl-phenyl}-propyl)-2-methyl-phenoxymethyl]-dihydro-furan-2-one

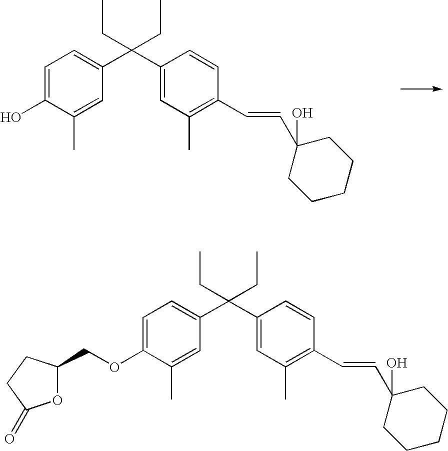

Using the same procedure as described for the preparation of Example 1-(6), the title compound was prepared from 4-(1-ethyl-1-{4-[(E)-2-(1-hydroxy-cyclohexyl)-vinyl]-3-methyl-phenyl}-propyl)-2-methyl-phenol (compound prepared in Example 11). The yield was 71%.

¹H-NMR(300 MHz, CDCl₃): 0.60 (t, 6H), 1.61-1.79 (m, 6H), 1.99-2.09 (m, 8H), 2.14 (s, 3H), 2.26 (m, 1H), 2.30 (s, 3H), 2.39-2.62 (m, 3H), 2.71-2.82 (m, 1H), 4.08 (dd, 1H), 4.14 (dd, 1H), 4.84-4.91 (m, 1H), 6.20 (d, 1H), 6.66 (d, 1H), 6.81 (d, 1H), 6.89-6.97 (m, 5H), 7.32 (d, 1H).

### (2) Preparation of (S)-5-[4-(1-ethyl-1-{4-[(E)-2-(1-hydroxy-cyclohexyl)-vinyl]-3-methyl-phenyl}-propyl)-2-methyl-phenoxy]-4-hydroxy-pentanoic acid

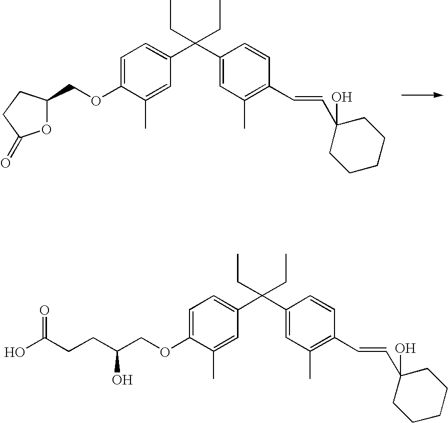

Using the same procedure as described for the preparation of Example 1-(7), the title compound was prepared from (S)-5-[4-(1-ethyl-1-{4-[(E)-2-(1-hydroxy-cyclohexyl)-vinyl]-3-methyl-phenyl}-propyl)-2-methyl-phenoxymethyl]-dihydro-furan-2-one (compound prepared in Example 37-(1)). The yield was 98%.

¹H-NMR (300 MHz, CD₃OD as potassium salt): 0.60 (t, 6H), 1.30-1.37 (m, 1H), 1.56-2.02 (m, 11H), 2.06 (q, 4H), 2.15 (s, 3H), 2.28 (s,3H), 2.37 (td, 2H), 3.89-3.98 (m, 1H), 6.16 (d, 1H), 6.76-6.98 (m, 2H), 6.84 (d, 1H), 6.93-6.98 (m, 3H), 7.30 (d, 1H); MS (ESI−): 507 ([M−H]⁻).

### Example 38

### Preparation of 5-(4-{1-ethyl-1-[4-(3-hydroxy-4,4-dimethyl-heptyl)-3-methyl-phenyl]-propyl}-2-methyl-phenoxy)-4(S)-hydroxy-pentanoic acid

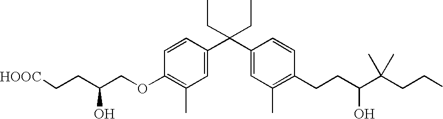

### (1) Preparation of 4-{1-ethyl-1-[4-(3-hydroxy-4,4-dimethyl-hept-6-en-1-ynyl)-3-methyl-phenyl]-propyl}-2-methyl-phenol

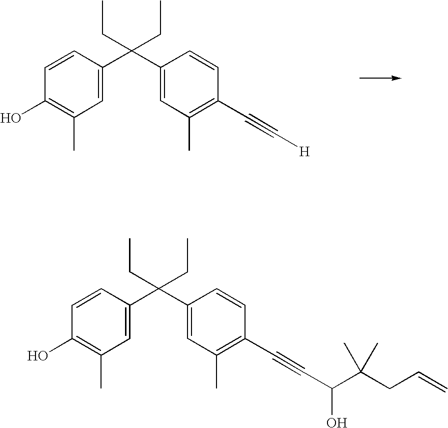

To a solution of 4-[1-ethyl-1-(4-ethynyl-3-methyl-phenyl)-propyl]-2-methyl-phenol (compound prepared in Example 1-(3)) (1 g, 3.42 mmol) in THF (20 ml) was added 1.6N n-BuLi in Hex (5.34 ml, 8.55 mmol) at −78 degrees C. under nitrogen atmosphere. The mixture was stirred at −78 degrees C. for 1 h and 2,2-dimethyl-4-pentenal (1.39 ml, 10.26 mmol) was added. The reaction mixture was warmed to room temperature and stirred for 12 h. The mixture was poured into saturated NH₄Cl aq. and the products were extracted with EtOAc. The extracts were washed with brine, dried over MgSO₄, filtered and concentrated under reduced pressure. The obtained residue was purified by Silica gel chromatography (EtOAc:n-Hexane=1:12) to give the title compound (829 mg, 60%).

¹H-NMR (300 MHz, CDCl₃): 0.59 (t, 6H), 1.04 (d, 6H), 2.03 (q, 4H), 2.18 (s, 3H), 2.20 (m, 2H), 2.38 (s, 3H), 4.34 (s, 1H), 5.10 (m, 2H), 5.87 (m, 1H), 6.65 (d, 1H), 6.80-7.00 (m, 4H), 7.28 (d, 1H); MS (ESI−): 403 ([M−H]⁻).

### (2) Preparation of 4-{1-ethyl-1-[4-(3-hydroxy-4,4-dimethyl-heptyl)-3-methyl-phenyl]-propyl}-2-methyl-phenol

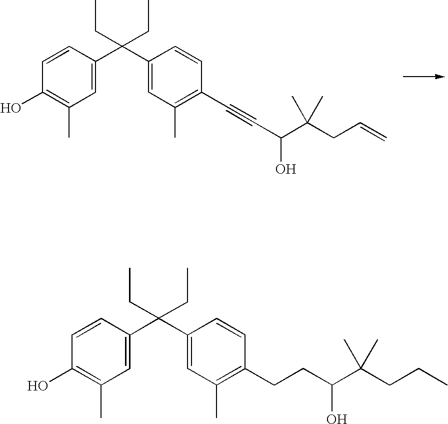

To a solution of4-{1-ethyl-1-[4-(3-hydroxy-4,4-dimethyl-hept-6-en-1l-ynyl)-3-methyl-phenyl]-propyl}-2-methyl-phenol (compound prepared in Example 38-(1)) (830 mg, 2.05 mmol) in MeOH (20 ml) was added 10% palladium on activated carbon (100 mg) and the mixture was stirred at room temperature under H₂ gas for 3 h. The mixture was filtered through celite and concentrated under reduced pressure. The obtained residue was purified by flash chromatography on silica gel (n-Hexane:EtOAc=10:1) to give the title compound (670 mg, 80%).

¹H-NMR (300 MHz, CDCl₃): 0.60 (t, 6H), 0.85 (m, 9H), 1.21 (m, 4H), 1.52 (m, 1H), 1.77 (m, 1H), 2.03 (q, 4H), 2.19 (s, 3H), 2.24 (s, 3H), 2.55 (m, 1H), 2.85 (m, 1H), 3.32 (d, 1H), 6.64 (d, 1H), 6.84-7.03 (m, 5H).

### (3) Preparation of (S)-5-(4-{1-ethyl-1-[4-(3-hydroxy-4,4-dimethyl-heptyl)-3-methyl-phenyl]-propyl}-2-methyl-phenoxymethyl)-dihydro-furan-2-one

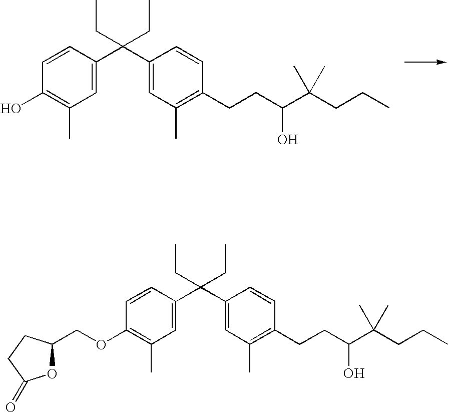

Using the same procedure as described for the preparation of Example 1-(6), the title compound was prepared from 4-{1-ethyl-1-[4-(3-hydroxy-4,4-dimethyl-heptyl)-3-methyl-phenyl]-propyl}-2-methyl-phenol (compound prepared in Example 38-(2)). The yield was 52%.

¹H-NMR (300 MHz, CDCl₃): 0.59 (t, 6H), 0.86 (m, 9H), 1.24 (m, 4H), 1.50 (m, 1H), 1.77 (m, 1H), 2.03 (q, 4H), 2.15 (s, 3H), 2.25 (s, 3H), 2.29-2.62 (m, 4H), 2.71-2.89 (m, 2H), 3.30 (d, 1H), 4.12 (m, 2H), 4.88 (m, 1H), 6.66 (d, 1H), 6.91 (m, 4H), 7.02 (d, 1H).

### (4) Preparation of 5-(4-{1-ethyl-1-[4-(3-hydroxy-4,4-dimethyl-heptyl)-3-methyl-phenyl]-propyl}-2-methyl-phenoxy)-4(S)-hydroxy-pentanoic acid

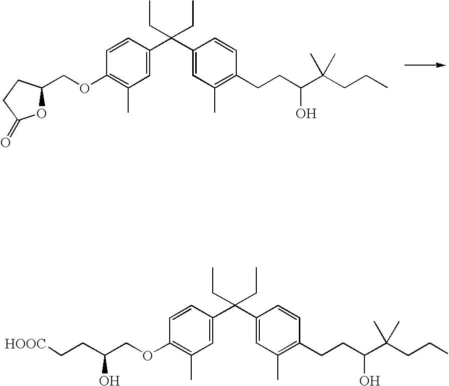

Using the same procedure as described for the preparation of Example 1-(7), the title compound was prepared from (S)-5-(4-{1-ethyl-1-[4-(3-hydroxy-4,4-dimethyl-heptyl)-3-methyl-phenyl]-propyl}-2-methyl-phenoxymethyl)-dihydro-furan-2-one (compound prepared in Example 38-(3)). The yield was 90%.

¹H-NMR (300 MHz, CD₃OD as potassium salt): 0.59 (t, 6H), 0.82 (m, 9H), 1.21 (m, 4H), 1.50 (m, 1H), 1.70 (m, 1H), 1.89 (m, 2H), 2.05 (q, 4H), 2.14 (s, 3H), 2.24 (s, 3H), 2.37 (t, 2H), 2.54 (m, 1H), 2.86 (m, 1H), 3.22 (d, 1H), 3.94 (m, 3H), 6.76 (d, 1H), 6.92 (m, 4H), 7.01 (d, 1H); MS (ESI−): 525 ([M−H]⁻).

### Example 39

### Preparation of 5-(4-{1-ethyl-1-[4-(3-hydroxy-4,4-dimethyl-octyl)-3-methyl-phenyl]-propyl}-2-methyl-phenoxy)-4(S)-hydroxy-pentanoic acid

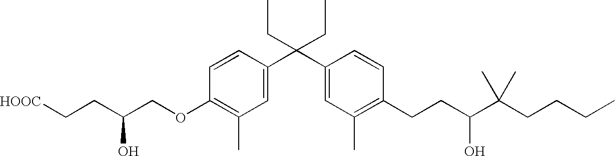

### (1) Preparation of 4-{1-ethyl-1-[4-(3-hydroxy-4,4-dimethyl-oct-1-ynyl)-3-methyl-phenyl]-propyl}-2-methyl-phenol

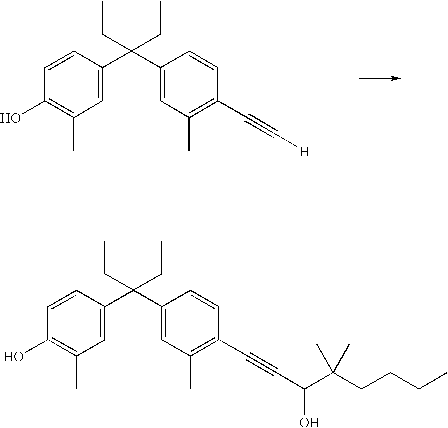

Using the same procedure as described for the preparation of Example 38-(1), the title compound was prepared from 4-[1-ethyl-1-(4-ethynyl-3-methyl-phenyl)-propyl]-2-methyl-phenol (compound prepared in Example 1-(3)) and 2,2-dimethyl hexanal. The yield was 24%.

¹H-NMR (300 MHz, CDCl₃): 0.59 (t, 6H), 0.91 (t, 3H), 1.01 (s,3H), 1.03 (s, 3H), 1.30 (m, 6H), 2.03 (q, 4H), 2.19 (s, 3H), 2.38 (s, 3H), 4.33 (m, 1H), 6.65 (d, 1H), 6.83-7.00 (m, 4H), 7.29 (d, 1H).

### (2) Preparation of 4-{1-ethyl-1-[4-(3-hydroxy-4,4-dimethyl-octyl)-3-methyl-phenyl]-propyl}-2-methyl-phenol

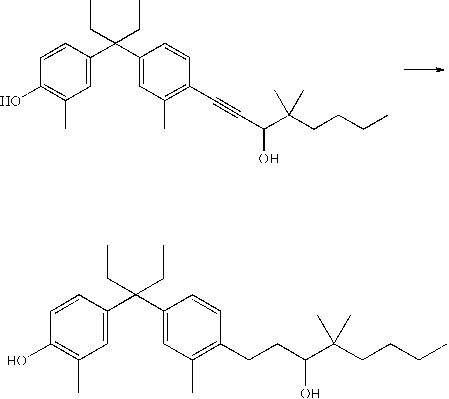

Using the same procedure as described for the preparation of Example 38-(2), the title compound was prepared from 4-{1-ethyl-1-[4-(3-hydroxy-4,4-dimethyl-oct-1-ynyl)-3-methyl-phenyl]-propyl}-2-methyl-phenol (compound prepared in Example 39-(1)). The yield was 86%.

¹H-NMR (300 MHz, CDCl₃): 0.60 (t, 6H), 0.86 (m, 9H), 1.26 (m, 6H), 1.52 (m, 1H), 1.78 (m, 1H), 2.03 (q, 4H), 2.20 (s, 3H), 2.25 (s, 3H), 2.55 (m, 1H), 2.85 (m, 1H), 3.32 (m, 1H), 6.64 (d, 1H), 6.84-6.92 (m, 4H), 7.02 (d, 1H).

### (3) Preparation of (S)-5-(4-{1-ethyl-1-[4-(3-hydroxy-4,4-dimethyl-octyl)-3-methyl-phenyl]-propyl}-2-methyl-phenoxymethyl)-dihydro-furan-2-one

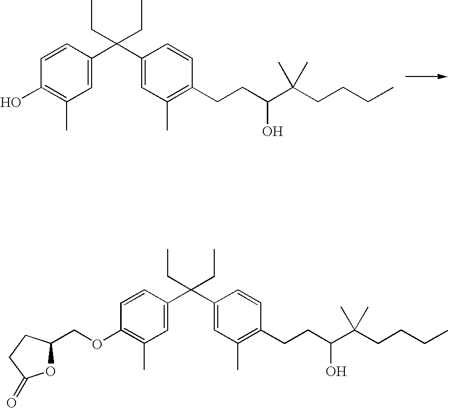

Using the same procedure as described for the preparation of Example 1-(6), the title compound was prepared from 4-{1-ethyl-1-[4-(3-hydroxy-4,4-dimethyl-octyl)-3-methyl-phenyl]-propyl}-2-methyl-phenol (compound prepared in Example 39-(2)). The yield was 58%.

¹H-NMR (300 MHz, CDCl₃): 0.59 (t, 6H), 0.85 (m, 9H), 1.23 (m, 6H), 1.52 (m, 1H), 1.76 (m, 1H), 2.03 (q, 4H), 2.15 (s, 3H), 2.25 (s, 3H), 2.29-2.62 (m, 4H), 2.71-2.88 (m, 2H), 3.31 (d, 1H), 4.11 (m, 2H), 4.87 (m, 1H), 6.66 (d, 1H), 6.92 (m, 4H), 7.02 (d, 1H).

### (4) Preparation of 5-(4-{1-ethyl-1-[4-(3-hydroxy-4,4-dimethyl-octyl)-3-methyl-phenyl]-propyl}-2-methyl-phenoxy)-4(S)-hydroxy-pentanoic acid

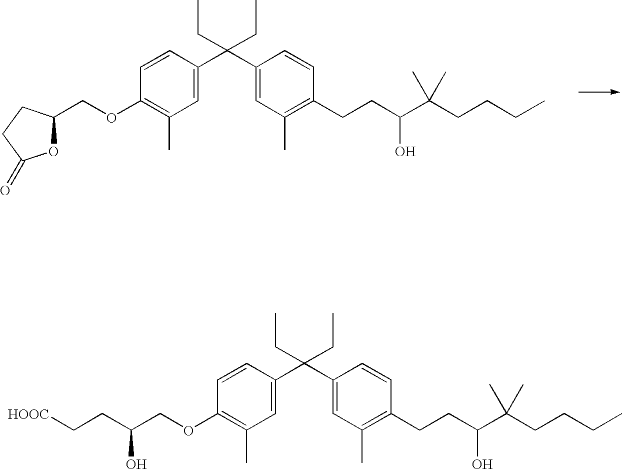

Using the same procedure as described for the preparation of Example 1-(7), the title compound was prepared from (S)-5-(4-{1-ethyl-1-[4-(3-hydroxy-4,4-dimethyl-octyl)-3-methyl-phenyl]-propyl}-2-methyl-phenoxymethyl)-dihydro-furan-2-one (compound prepared in Example 39-(3)). The yield was 92%.

¹H-NMR (300 MHz, CD₃OD as potassium salt): 0.59 (t, 6H), 0.84 (m, 9H), 1.25 (m, 6H), 1.51 (m, 1H), 1.73 (m, 1H), 1.89 (m, 2H), 2.05 (q, 4H), 2.15 (s, 3H), 2.24 (s, 3H), 2.37 (t, 2H), 2.55 (m, 1H), 2.86 (m, 1H), 3.23 (d, 1H), 3.94 (m, 3H), 6.76 (d, 1H), 6.92 (m, 4H), 7.02 (d, 1H); MS (ESI−): 539 ([M−H]⁻).

### Example 40

### Preparation of 5-(4-{1-ethyl-1-[4-(3-hydroxy-4,4-dimethyl-decyl)-3-methyl-phenyl]-propyl}-2-methyl-phenoxy)-4(S)-hydroxy-pentanoic acid

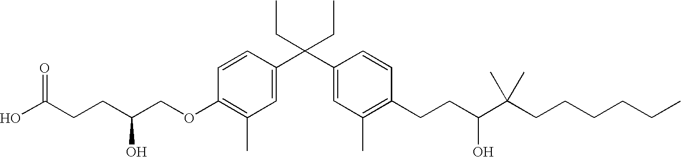

### (1) Preparation of 2,2-dimethyl-octanoic acid ethyl ester

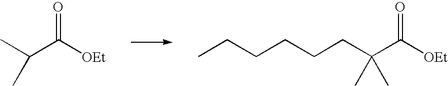

To a solution of Isobutyric acid ethyl ester (4.3 g, 37.02 mmol) in anhydrous THF (37 ml) under nitrogen atmosphere at −78 degrees C. was added LDA (6.7 ml, 10.65 mmol) and the mixture was stirred for 30 min. To the mixture, iodohexane (6 ml, 40.72 mmol.) was added and the mixture was stirred at −78 degrees C. for 5 h. Then the mixture was warmed to room temperature and poured into 1N—HCl and the products were extracted with diethyl ether. The extracts were washed with brine, dried over MgSO₄, filtered, and concentration in vacuo. The obtained residue was purified by distillation (78˜82 degrees C., 12 torr) to give the title compound (6.5 g, 87%).

¹H-NMR (300 MHz, CDCl₃): 0.87 (t, 3H), 1.15 (s, 6H), 1.21-1.40 (m, 10H), 1.50 (m, 2H), 4.10 (q, 2H).

### (2) Preparation of 2,2-dimethyl-octan-1-ol

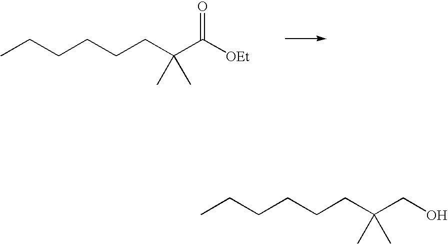

To a solution of 2,2-dimethyl-octanoic acid ethyl ester (compound prepared in Example 40-(1)) (5.79 g, 28.90 mmol) in anhydrous THF (29 ml) at 0 degrees C. under nitrogen atmosphere was added 1N—LiAlH₄/THF (14.5 ml, 14.50 mmol) and the mixture was stirred at 0 degrees C. for 4 h. Then the mixture was warmed to room temperature and quenched with H₂O, 10% NaOH solution and H₂O. The reaction mixture was filtered through celite and the filtrate was washed with brine, dried over MgSO₄, filtered, and concentrated in vacuo. The obtained residue was purified by silica gel chromatography eluting with dichloromethane to give the title compound (3.12 g, 69.0%).

¹H-NMR(300MHz, CDCl₃): 0.86(s, 6H), 0.88(t, 3H), 1.25 (m, 1H), 3.31(s, 2H).

### (3) Preparation of 2,2-dimethyl-octanal

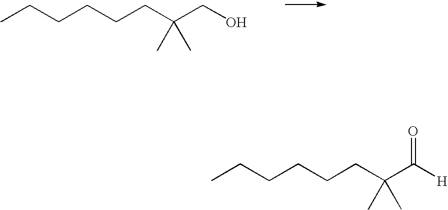

To a solution of 2,2-dimethyl-octan-1-ol (compound prepared in Example 40-(2)) (3.12 g, 19.71 mmol) in anhydrous dichloromethane (27.5 ml) under nitrogen atmosphere was added PCC (6.54 g, 30.35 mmol) and the mixture was stirred at room temperature for 3 h. The mixture was diluted with diethyl ether and filtered through a celite and silicagel and concentrated in vacuo. The obtained residue was purified by silica gel chromatography (hexane/EtOAc=10:1)to give the title compound (2.55 g, 82%).

¹H-NMR (300 MHz, CDCl₃): 0.88 (t, 3H), 1.04 (s, 6H), 1.15-1.29 (m, 9H), 1.43-1.48 (m, 2H), 9.21 (s, 1H).

### (4) Preparation of 4-{1-ethyl-1-[4-(3-hydroxy-4,4-dimethyl-dec-1-ynyl)-3-methyl-phenyl]-propyl}-2-methyl-phenol

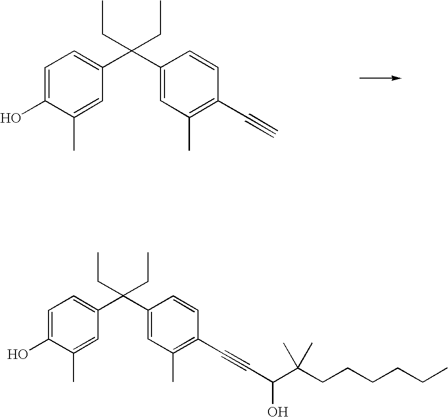

Using the same procedure as described for the preparation of Example 38-(1), the title compound was prepared from 4-[1-ethyl-1-(4-ethynyl-3-methyl-phenyl)-propyl]-2-methyl-phenol (compound prepared in Example 1-(3)) and 2,2-dimethyl-octanal (compound prepared in Example 40-(3)). The yield was 58%.

¹H-NMR (300 MHz, CDCl₃): 0.59 (t, 6H), 0.88 (t, 3H), 1.01 (s,3H), 1.02 (s, 3H), 1.25-1.49 (m, 10H), 1.75 (d, 1H), 2.02 (q, 4H), 2.19 (s, 3H), 2.38 (s, 3H), 4.32 (d, 1H), 4.52 (s, 1H). 6.65 (d, 1H), 6.82-6.86 (m, 2H), 6.93 (dd, 1H), 7.00 (s, 1H), 7.28 (d, 1H).

### (5) Preparation of 4-{1-ethyl-1-[4-(3-hydroxy-4,4-dimethyl-decyl)-3-methyl-phenyl]-propyl}-2-methyl-phenol

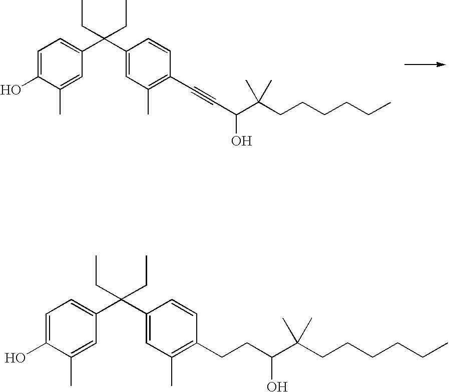

Using the same procedure as described for the preparation of Example 38-(2), the title compound was prepared from 4-{1-ethyl-1-[4-(3-hydroxy-4,4-dimethyl-dec-1-ynyl)-3-methyl-phenyl]-propyl}-2-methyl-phenol (compound prepared in Example 40-(4)). The yield was 81%.

¹H-NMR (300 MHz, CDCl₃): 0.59 (t, 6H), 0.86 (t, 9H), 1.17-1.29 (m, 9H), 1.42-1.60 (m, 2H), 1.72-1.83 (m, 1H), 2.03 (q, 4H), 2.19 (s, 3H), 2.24 (s, 3H), 2.50-2.60 (m, 1 H), 2.82-2.91 (m, 1H), 3.34 (d, 1H), 5.01 (s, 1H), 6.64 (d, 1H), 6.84 (dd, 1H), 6.90-6.92 (m, 2H), 7.02 (s, 1H), 7.26 (d, 1H).

### (6) Preparation of (S)-5-(4-{1-ethyl-1-[4-(3-hydroxy-4,4-dimethyl-decyl)-3-methyl-phenyl]-propyl}-2-methyl-phenoxymethyl)-dihydro-furan-2-one

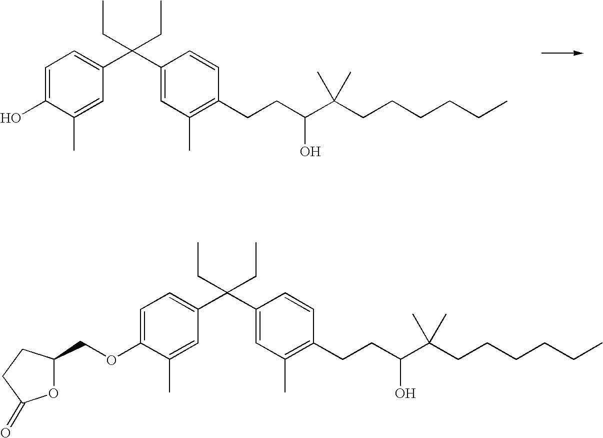

Using the same procedure as described for the preparation of Example 1-(6), the title compound was prepared from 4-{1-ethyl-1-[4-(3-hydroxy-4,4-dimethyl-decyl)-3-methyl-phenyl]-propyl}-2-methyl-phenol (compound prepared in Example 40-(5)). The yield was 67%.

¹H-NMR (300 MHz, CDCl₃): 0.59 (t, 6H), 0.83 (s, 3H), 0.85 (s, 3H), 0.88 (t, 3H), 1.25-1.49 (m, 11H), 1.40-1.52 (m, 1H), 1.78 (m, 1H), 2.03 (q, 4H), 2.15 (s, 3H), 2.25 (s, 3H), 2.29-2.62 (m, 4H), 2.71-2.88 (m, 2H), 3.30 (d, 1H), 4.04-4.19 (m, 2H), 4.87 (m, 1H), 6.66 (d, 1H), 6.92 (m, 4H), 7.02 (d, 1H); MS (ESI+): 573 ([M+Na]⁺).

### (7) Preparation of 5-(4-{1-ethyl-1-[4-(3-hydroxy-4,4-dimethyl-decyl)-3-methyl-phenyl]-propyl}-2-methyl-phenoxy)-4(S)-hydroxy-pentanoic acid

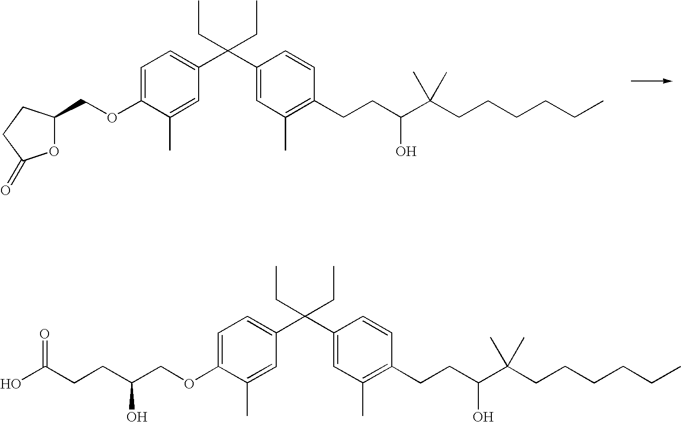

Using the same procedure as described for the preparation of Example 1-(7), the title compound was prepared from (S)-5-(4-{1-ethyl-1-[4-(3-hydroxy-4,4-dimethyl-decyl)-3-methyl-phenyl]-propyl)-2-methyl-phenoxymethyl)-dihydro-furan-2-one (compound prepared in Example 40-(6)). The yield was 95%.

¹H-NMR (300 MHz, CD₃OD as potassium salt): 0.58 (t, 6H), 0.80 (s, 3H), 0.84 (s, 3H), 0.89 (t, 3H), 1.16-1.33 (m, 10H), 1.46-1.52 (m, 1H), 1.68-2.00 (m, 4H), 2.05 (q, 4H), 2.14 (s, 3H), 2.24 (s,3H), 2.37-2.40 (td, 2H), 2.55-2.57 (m, 1H), 2.84-2.85 (m,1H), 3.23 (d, 1H), 3.88-4.01 (m, 3H), 6.75 (d, 1H), 6.84 (s, 1H), 6.84-6.95 (m, 3H), 7.01 (d, 1H); MS (ESI+): 591 ([M+Na]⁺).

### Example 41

### Preparation of 5-(4-{1-ethyl-1-[4-(3-hydroxy-4,4-dimethyl-nonyl)-3-methyl-phenyl]-propyl}-2-methyl-phenoxy)-4(S)-hydroxy-pentanoic acid

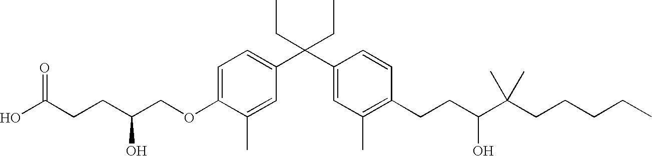

### (1) Preparation of 2,2-dimethyl-heptanoic acid ethyl ester

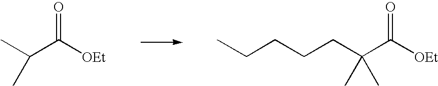

Using the same procedure as described for the preparation of Example 40-(1), the title compound was prepared from Isobutyric acid ethyl ester and iodopentane. The yield was 86%.

¹H-NMR (300 MHz, CDCl₃): 0.87 (t, 3H), 1.16 (s, 6H), 1.21-1.40 (m, 8H), 1.54 (m, 2H), 4.14 (q, 2H).

### (2) Preparation of 2,2-dimethyl-heptan-1-ol

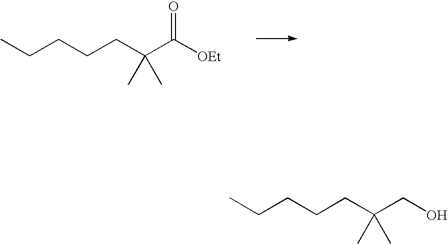

Using the same procedure as described for the preparation of Example 40-(2), the title compound was prepared from 2,2-dimethyl-heptanoic acid ethyl ester (compound prepared in Example 41-(1)). The yield was 89%.

¹H-NMR (300 MHz, CDCl₃): 0.86 (s, 6H), 0.89 (t, 3H), 1.23-1.35 (m, 9H), 3.31 (s, 2H).

### (3) Preparation of 2,2-dimethyl-heptanal

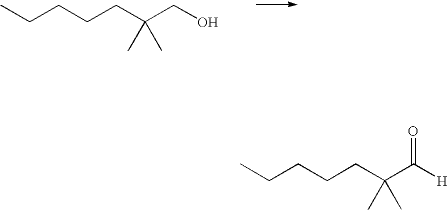

Using the same procedure as described for the preparation of Example 40-(3), the title compound was prepared from 2,2-dimethyl-heptan-1-ol (compound prepared in Example 41-(2)). The yield was 83%.

¹H-NMR (300 MHz, CDCl₃): 0.88 (t, 3H), 1.05 (s, 6H), 1.20-1.31 (m, 6H), 1.42-1.48 (m, 2H), 9.45 (s, 1H).

### (4) Preparation of 4-{1-ethyl-1-[4-(3-hydroxy-4,4-dimethyl-non-1-ynyl)-3-methyl-phenyl]-propyl}-2-methyl-phenol

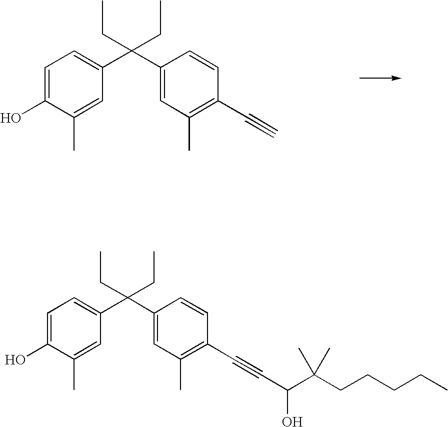

Using the same procedure as described for the preparation of Example 38-(1), the title compound was prepared from 4-[1-ethyl-1-(4-ethynyl-3-methyl-phenyl)-propyl]-2-methyl-phenol (compound prepared in Example 1-(3)) and 2,2-dimethyl-heptanal (compound prepared in Example 41-(3)). The yield was 80%.

¹H-NMR (300 MHz, CDCl₃): 0.59 (t, 6H), 0.86 (t, 3H), 1.01 (s,3H), 1.03 (s, 3H), 1.25-1.49 (m, 8H), 1.75 (m, 1H), 2.02 (q, 4H), 2.19 (s, 3H), 2.38 (s, 3H), 4.33 (d, 1 H), 4.60 (s,1 H). 6.65 (d, 1H), 6.86 (dd, 2H), 6.94 (dd, 1 H), 7.01 (s,1 H), 7.27 (d, 1H).

### (5) Preparation of 4-{1-ethyl-1-[4-(3-hydroxy-4,4-dimethyl-nonyl)-3-methyl-phenyl]-propyl}-2-methyl-phenol

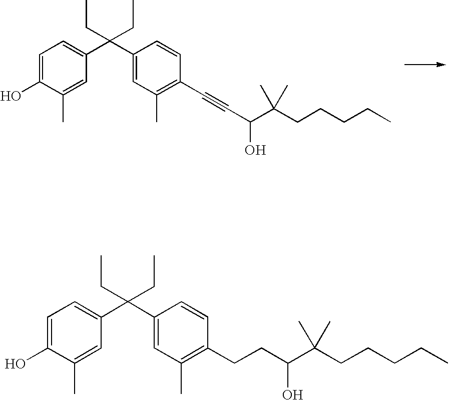

Using the same procedure as described for the preparation of Example 38-(2), the title compound was prepared from 4-{1-ethyl-1-[4-(3-hydroxy-4,4-dimethyl-non-1-ynyl)-3-methyl-phenyl]-propyl}-2-methyl-phenol (compound prepared in Example 41-(4)). The yield was 81%.

¹H-NMR (300 MHz, CDCl₃): 0.59 (t, 6H), 0.86 (t, 9H), 1.17-1.29 (m, 9H), 1.42-1.60 (m, 2H), 1.72-1.83 (m, 1H), 2.03 (q, 4H), 2.19 (s, 3H), 2.24 (s, 3H), 2.50-2.55 (m, 1H), 2.82-2.91 (m, 1H), 3.34 (dd, 1H), 5.01 (s, 1H), 6.64 (d, 1H), 6.84 (dd, 2H), 6.90-6.92 (m, 3H), 7.01 (s, 1H), 7.27 (d, 1H).

### (6) Preparation of (S)-5-(4-{1-ethyl-1-[4-(3-hydroxy-4,4-dimethyl-nonyl )-3-methyl-phenyl]-propyl}-2-methyl-phenoxymethyl)-dihydro-furan-2-one

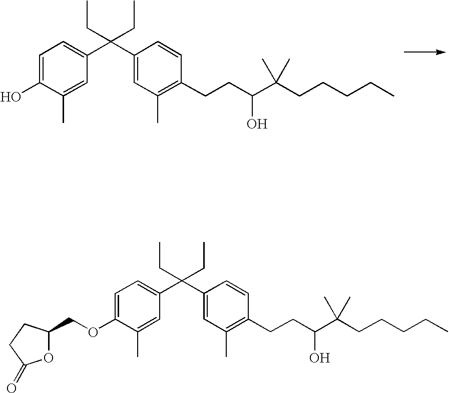

Using the same procedure as described for the preparation of Example 1-(6), the title compound was prepared from 4-{1-ethyl-1-[4-(3-hydroxy-4,4-dimethyl-nonyl)-3-methyl-phenyl]-propyl}-2-methyl-phenol (compound prepared in Example 41-(5)). The yield was 65%.

¹H-NMR (300 MHz, CDCl₃): 0.59 (t, 6H), 0.83 (s, 3H), 0.85 (s, 3H), 0.87 (t, 3H), 1.15-1.33 (m, 10H), 1.42-1.54 (m, 1H), 1.71-1.83 (m, 1H), 2.03 (q, 4H), 2.15 (s, 3H), 2.25 (s, 3H), 2.29-2.61 (m, 4H), 2.71-2.88 (m, 2H), 3.30 (d, 1H), 4.11 (m, 2H), 4.87 (m, 1H), 6.65 (d, 1H), 6.89-6.97 (m, 4H), 7.02 (d, 1H).

### (7) Preparation of 5-(4-{1-ethyl-1-[4-(3-hydroxy-4,4-dimethyl-nonyl)-3-methyl-phenyl]-propyl}-2-methyl-phenoxy)-4(S)-hydroxy-pentanoic acid

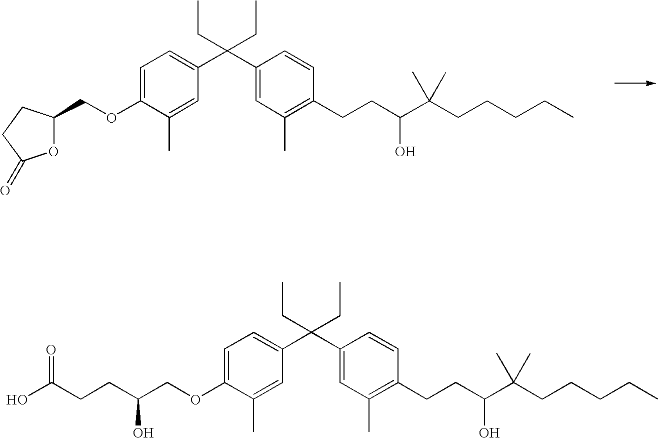

Using the same procedure as described for the preparation of Example 1-(7), the title compound was prepared from (S)-5-(4-{1-ethyl-1-[4-(3-hydroxy-4,4-dimethyl-nonyl)-3-methyl-phenyl]-propyl}-2-methyl-phenoxymethyl)-dihydro-furan-2-one (compound prepared in Example 41-(6)). The yield was 92%.

¹H-NMR (300 MHz, CD₃OD as potassium salt): 0.58 (t, 6H), 0.80 (s, 3H), 0.84 (s, 3H), 0.89 (t, 3H), 1.16-1.33 (m, 8H), 1.46-1.52 (m, 1H), 1.65-2.00 (m, 1H), 2.05 (q, 4H), 2.14 (s, 3H), 2.24 (s,3H), 2.37-2.40 (td, 2H), 2.55-2.57 (m, 1H), 2.84-2.85 (m, 1H), 3.23 (d, 1H), 3.90-3.91 (m, 3H), 6.75 (d, 1H), 6.84 (s, 1H), 6.84-6.97 (m, 3H), 7.01 (d, 1H); MS (ESI−): 553 ([M−H]⁻).

### Example 42

### Preparation of (R)-5-(4-{1-ethyl-1-[4-(3-ethyl-3-hydroxy-pent-1-ynyl)-3-methyl-phenyl]-propyl}-2-methyl-phenoxymethyl)-dihydro-furan-2-one

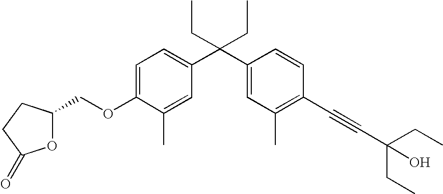

### Preparation of (R)-5-(4-{1-ethyl-1-[4-(3-ethyl-3-hydroxy-pent-1-ynyl)-3-methyl-phenyl]-propyl}-2-methyl-phenoxymethyl)-dihydro-furan-2-one

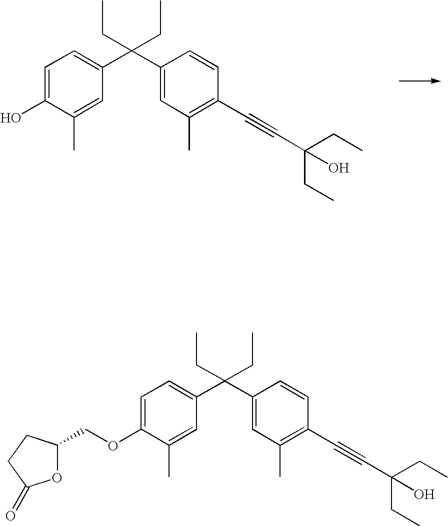

Using the same procedure as described for the preparation of Example 1-(6), the title compound was prepared from 4-{1-ethyl-1-[4-(3-ethyl-3-hydroxy-pent-1-ynyl)-3-methyl-phenyl]-propyl}-2-methyl-phenol (compound prepared in Example. 1-(4)) and Toluene-4-sulfonic acid (R)-5-oxo-tetrahydro-furan-2-ylmethyl ester. The yield was 51%.

¹H-NMR (300 MHz, CDCl₃): 0.58 (t, 6H), 1.11 (t, 6H), 1.76 (q, 4H), 2.02 (q, 4H), 2.14 (s, 3H), 2.36 (s, 3H), 2.22-2.61 (m, 3H), 2.71-2.83 (m, 1H), 4.06 (dd, 1H), 4.14 (dd, 1H), 4.87 (m, 1H), 6.65 (d, 1H), 6.87 (s, 1H), 6.92 (d, 2H), 6.98 (s, 1H), 7.27 (d, 1H); MS (ESI+): 499 ([M+Na]⁺).

### Example 43

### Preparation of 5-(4-{1-ethyl-1-[4-(3-ethyl-3-hydroxy-pent-1-ynyl)-3-methyl-phenyl]-propyl}-2-methyl-phenoxy)-4(R)-hydroxy-pentanoic acid

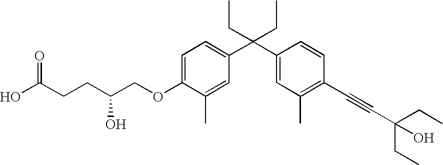

### Preparation of 5-(4-{1-ethyl-1-[4-(3-ethyl-3-hydroxy-pent-1-ynyl)-3-methyl-phenyl]-propyl}-2-methyl-phenoxy)-4(R)-hydroxy-pentanoic acid

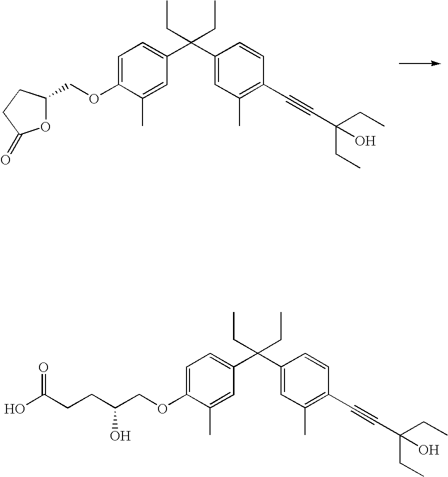

Using the same procedure as described for the preparation of Example 1-(7), the title compound was prepared from (R)-5-(4-{1-ethyl-1-[4-(3-ethyl-3-hydroxy-pent-1-ynyl)-3-methyl-phenyl]-propyl}-2-methyl-phenoxymethyl)-dihydro-furan-2-one (compound prepared in Example 42). The yield was 98%.

¹H-NMR (300 MHz, CD₃OD as potassium salt): 0.61 (t, 6H), 1.11 (t, 6H), 1.70-1.77 (m, 5H), 1.90-2.01 (m, 1H), 2.07 (q, 4H), 2.17 (s, 3H), 2.36 (s, 3H), 2.37 (td, 2H), 3.91-4.00 (m, 3H), 6.79 (d, 1H), 6.85 (s, 1H), 6.95 (d, 2H), 7.01 (s, 1H), 7.24 (d, 1H); MS (ESI−): 493 ([M−H]⁻).

### Example 44

### Preparation of (R)-5-(4-{1-ethyl-1-[4-((E)-3-ethyl-3-hydroxy-pent-1-enyl)-3-methyl-phenyl]-propyl}-2-methyl-phenoxymethyl)-dihydro-furan-2-one

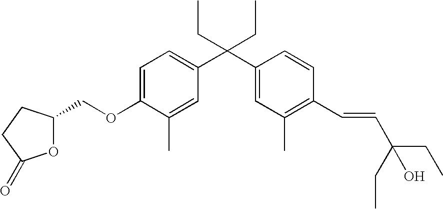

### Preparation of (R)-5-(4-{1-ethyl-1-[4-((E)-3-ethyl-3-hydroxy-pent-1-enyl)-3-methyl-phenyl]-propyl}-2-methyl-phenoxymethyl)-dihydro-furan-2-one

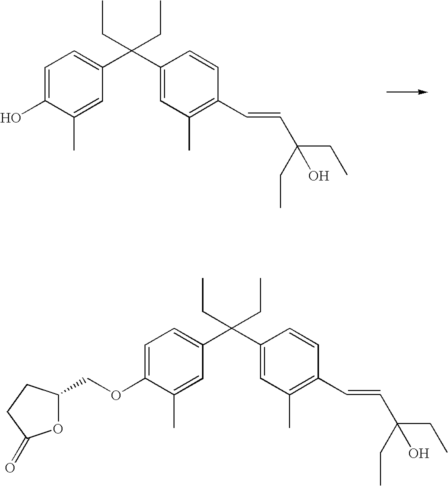

Using the same procedure as described for the preparation of Example 1-(6), the title compound was prepared from 4-{1-ethyl-1-[4-((E)-3-ethyl-3-hydroxy-pent-1-enyl)-3-methyl-phenyl]-propyl}-2-methyl-phenol (compound prepared in Example 1-(5)) and toluene4-sulfonic acid (R)-5-oxo-tetrahydro-furan-2-ylmethyl ester. The yield was 82%.

¹H-NMR (300 MHz, CDCl₃): 0.61 (t, 6H), 0.92 (t, 6H), 1.64 (q, 4H), 2.06 (q, 4H), 2.16 (s, 3H), 2.31 (s, 3H), 2.34-2.62 (m, 3H), 2.72-2.83 (m, 1H), 4.06 (dd, 1H), 4.15 (dd, 1H), 4.87 (m, 1H), 6.01 (d, 1H), 6.66 (d, 1H), 6.74 (d, 1H), 6.91-6.98 (m, 4H), 7.29 (d, 1H); MS (ESI+): 501 ([M+Na]⁺).

### Example 45

### Preparation of 5-(4-{1-ethyl-1-[4-((E)-3-ethyl-3-hydroxy-pent-1-enyl)-3-methyl-phenyl]-propyl}-2-methyl-phenoxy)-4(R)-hydroxy-pentanoic acid

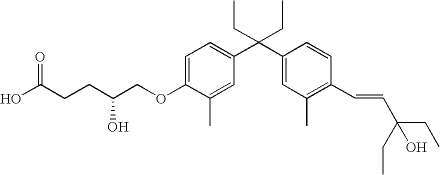

### Preparation of 5-(4-{1-ethyl-1-[4-((E)-3-ethyl-3-hydroxy-pent-1-enyl)-3-methyl-phenyl]-propyl}-2-methyl-phenoxy)-4(R)-hydroxy-pentanoic acid

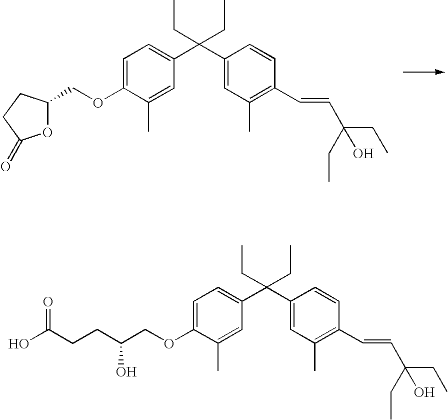

Using the same procedure as described for the preparation of Example 1-(7), the title compound was prepared from (R)-5-(4-{1-ethyl-1-[4-((E)-3-ethyl-3-hydroxy-pent-1-enyl)-3-methyl-phenyl]-propyl}-2-methyl-phenoxymethyl)-dihydro-furan-2-one (compound prepared in Example 44). The yield was 91%.

¹H-NMR (300 MHz, CD₃OD as potassium salt): 0.60 (t, 6H), 0.92 (t, 6H), 1.63 (q, 4H), 1.81-2.01 (m, 3H), 2.06 (q, 4H), 2.16 (s, 3H), 2.28 (s, 3H), 2.38 (td, 2H), 3.89-3.99 (m, 3H), 6.01 (d,1H), 6.74 (d,1H), 6.79 (s, 1H), 6.86 (s,1H), 6.93-6.95 (m, 3H), 7.29 (d,1H); MS (ESI−): 495 ([M−H]⁻).

### Example 46

### Preparation of (R)-5-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one

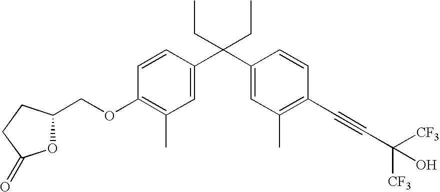

### (1) Preparation of (R)-5-(4-{1-ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one

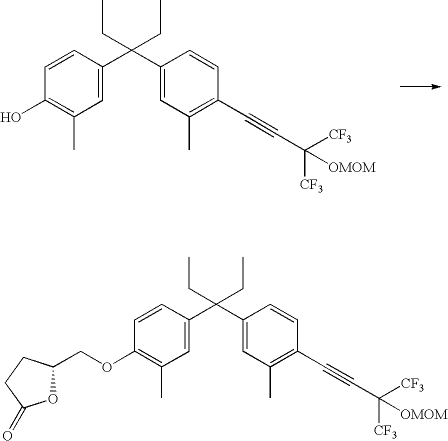

Using the same procedure as described for the preparation of Example 1-(6), the title compound was prepared from 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) and toluene-4-sulfonic acid (R)-5-oxo-tetrahydro-furan-2-ylmethyl ester. The yield was 42%.

### ¹H-NMR (300 MHz, CDCl₃): 0.59 (t, 6H), 2.04 (q, 4H), 2.15 (s, 3H), 2.27-2.50 (m, 2H), 2.39 (s, 3H), 2.56 (m, 1H), 2.77 (m, 1H), 3.48 (s, 3H), 4.12 (m, 2H), 4.88 (m, 1H), 5.15 (s, 2H), 6.67 (d, 1H), 6.85-7.03 (m, 4H), 7.37 (d, 1H).

### (2) Preparation of (R)-5-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one

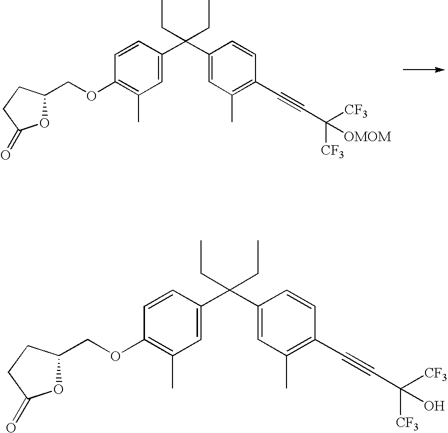

Using the same procedure as described for the preparation of Example 2-(4), the title compound was prepared from (R)-5-(4-{1-ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one (compound prepared in Example 46-(1)). The yield was 54%.

### ¹H-NMR (300 MHz, CDCl₃): 0.59 (t, 6H), 2.04 (q, 4H), 2.14 (s, 3H), 2.27-2.50 (m, 2H), 2.38 (s, 3H), 2.55 (m, 1H), 2.76 (m, 1H), 4.02 (s, 1H), 4.12 (m, 2H), 4.88 (m, 1H), 6.66 (d, 1H), 6.85-7.03 (m, 4H), 7.35 (d,1H); MS (ESI−): 555 ([M−H]⁻).

### Example 47

### Preparation of (R)-5-(4-{1-ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid

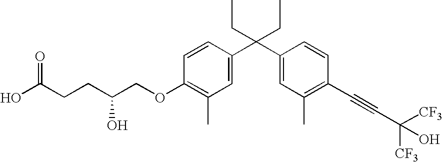

### Preparation of (R)-5-(4-{1-ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid

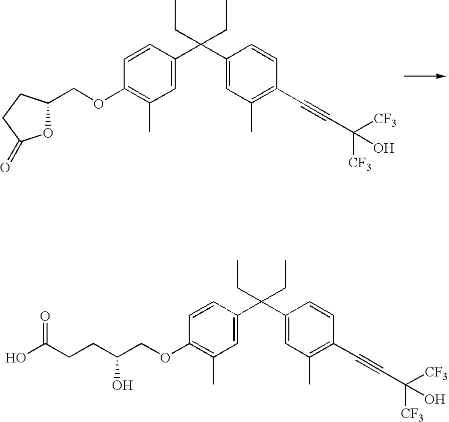

Using the same procedure as described for the preparation of Example 1-(7), the title compound was prepared from (R)-5-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one (compound prepared in Example 46-(2)). The yield was 96%.

¹H-NMR (300 MHz, CD₃OD as potassium salt): 0.59 (t, 6H), 1.80-2.00 (m, 2H), 2.07 (q, 4H), 2.15 (s, 3H), 2.35 (s, 3H), 2.38 (m, 2H), 3.94 (m, 3H), 6.78 (d, 1H), 6.84 (m,. 1H), 6.93-6.98 (m, 3H), 7.28 (d, 1H); MS (ESI−): 573 ([M−H]⁻).

### Example 48

### Preparation of (R)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one

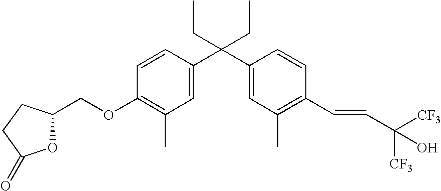

### (1) Preparation of (R)-5-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-dihydro-furan-2-one

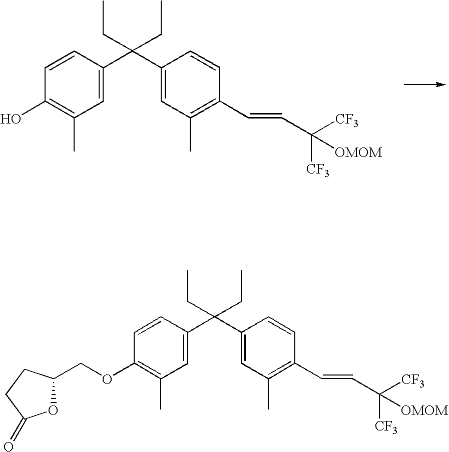

Using the same procedure as described for the preparation of Example 1-(6), the title compound was prepared from 4-{1-ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in Example 30-(1)) and toluene-4-sulfonic acid (R)-5-oxo-tetrahydro-furan-2-ylmethyl ester. The yield was 89%.

¹H-NMR (300 MHz, CDCl₃): 0.61 (t, 6H), 2.05 (q, 4H), 2.15 (s, 3H), 2.32 (s, 3H), 2.25-2.85 (m, 4H), 3.50 (s, 3H), 4.12 (m, 2H), 4.88 (m, 1H), 4.96 (s, 2H), 6.06 (d, 1H), 6.67 (d,1H), 6.96 (m, 4H), 7.34 (m, 2H).

### (2) Preparation of (R)-5-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one

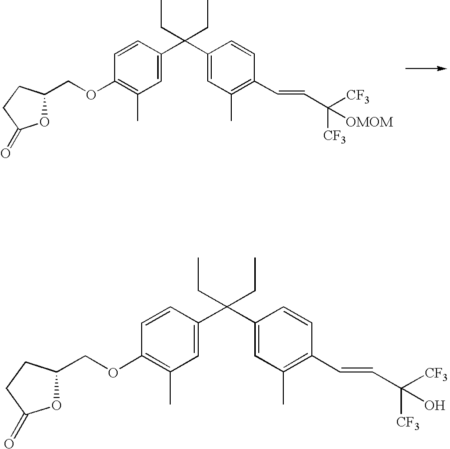

Using the same procedure as described for the preparation of Example 2-(4), the title compound was prepared from (R)-5-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-dihydro-furan-2-one (compound prepared in Example 48-(1)). The yield was 84%.

¹H-NMR (300 MHz, CDCl₃): 0.61 (t, 6H), 2.05 (q, 4H), 2.15 (s, 3H), 2.34 (s, 3H), 2.25-2.83 (m, 4H), 3.24 (s,1H), 4.11 (m, 2H), 4.88 (m,1H), 6.08 (d,1H), 6.67 (d, 1H), 6.95 (m, 4H), 7.35 (m, 2H); MS (ESI−): 557 ([M−H]⁻).

### Example 49

### Preparation of 5-(4-{1-ethyl-1-[3-methyl4-((E)4,4,4-trifluoro-3-hydroxy-3-trifluoromethyl-but-1-enyl)-phenyl]-propyl}-2-methyl-phenoxy)4(R)-hydroxy-pentanoic acid

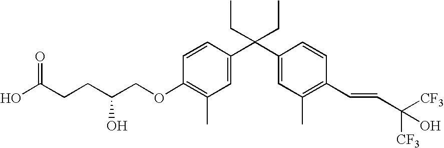

### Preparation of 5-(4-{1-ethyl-1-[3-methyl4-((E)-4,4,4-trifluoro-3-hydroxy-3-trifluoromethyl-but-1-enyl)-phenyl]-propyl}-2-methyl-phenoxy)4(R)-hydroxy-pentanoic acid

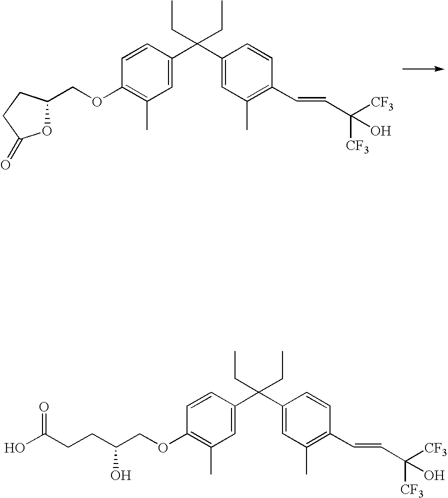

Using the same procedure as described for the preparation of Example 1-(7), the title compound was prepared from (R)-5-(4-{1-ethyl-1-[3-methyl4-((E)4,4,4-trifluoro-3-hydroxy-3-trifluoromethyl-but-1-enyl)-phenyl]-propyl}-2-methyl-phenoxymethyl)-dihydro-furan-2-one (compound prepared in Example 48-(2)). The yield was 93%.

¹H-NMR (300 MHz, CD₃OD as potassium salt): 0.60 (t, 6H), 1.80-2.20 (m, 2H), 2.07 (q, 4H), 2.15 (s, 3H), 2.32 (s, 3H), 2.37 (m, 2H), 3.94 (m, 3H), 6.18 (d, 1H), 6.77 (d, 1H), 6.86 (m, 1H), 6.97 (m, 3H), 7.37 (m, 2H); MS (ESI−): 575 ([M−H]⁻).

### Example 50

### Preparation of 5-(4-{1-ethyl-1-[4-(3-hydroxy-4,4-dimethyl-pentyl)-3-methyl-phenyl]-propyl}-2-methyl-phenoxy)-4(R)-hydroxy-pentanoic acid

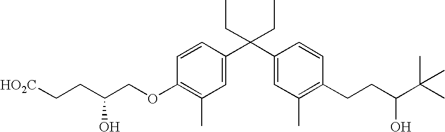

### Preparation of 5-(4-{1-ethyl-1-[4-(3-hydroxy-4,4-dimethyl-pentyl)-3-methyl-phenyl]-propyl}-2-methyl-phenoxy)-4(R)-hydroxy-pentanoic acid

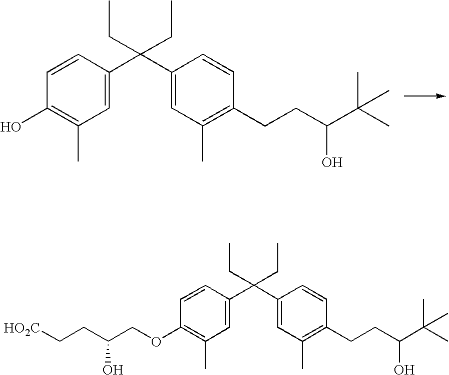

To a solution of 4-{1-ethyl-1-[4-(3-hydroxy-4,4-dimethyl-phenyl)-3-methyl-phenyl]-propyl}-2-methyl-phenol (compound prepared in Example 18) (190 mg, 0.50 mmol) in anhydrous DMF (5 ml) under nitrogen atmosphere were added K₂CO₃ (138.2 mg, 1.0 mmol) and (R)-toluene-4-sulfonic acid 5-oxo-tetrahydrofuran-2-ylmethyl ester (162.2 mg, 0.6 mmol) and the mixture was stirred overnight at 100 degrees C. The mixture was diluted with ethyl acetate, washed with sat. NH₄Cl, water and brine, dried over MgSO₄, filtered and concentrated under reduced pressure. The obtained residue was dissolved in methanol-H₂O (10:1, 5.5 ml). To the solution was added KOH as 1 tablet. After stirring for 30 min at room temperature, the mixture was concentrated under reduced pressure and the obtained residue was purified by silica-gel chromatography (hexane/EtOAc=4:1) to give the title compound (125 mg, 50%) as a solid.

¹H NMR (CDCl₃); 7.03-6.89 (m, 5H), 6.67 (d, 1H), 4.06 (m, 1H), 3.97 (ddd, 1H), 3.85 (ddd, 1H), 3.24 (dt, 1H), 2.86 (m, 1H), 2.64-2.50 (m, 3H), 2.25 (s, 3H), 2.17 (s, 3H), 2.04 (q, 4H), 1.93 (m, 2H), 1.79 (m, 1H), 1.50 (m, 1H), 0.88 (s, 9H), 0.59 (t, 6H); MS (ESI−): 497 ([M−H]⁻).

### Example 51

### Preparation of (S)-4-(4-{1-ethyl-1-[3-methyl-4-(4,4,4-trifluoro-3-hydroxy-3-trifluoromethyl-but-1-ynyl)-phenyl]-propyl}-2-methyl-phenoxy)-3-hydroxy-butyric acid

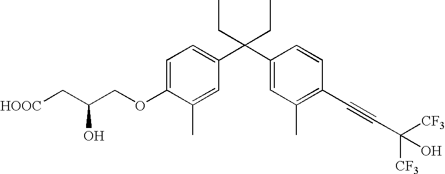

### (1) Preparation of 3(S)-(tert-Butyl-dimethyl-silanyloxy)-4-hydroxy-butyric acid ethyl ester

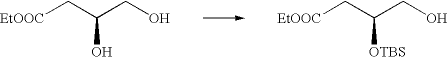

To a stirred solution of 3(S),4-Dihydroxy-butyric acid ethyl ester (Tetrahedron,1992, 48(20), 4067.) (569 mg, 3.8 mmol) in DMF (12 ml) were added TBSCl (1.7 g, 11.2 mmol) and imidazole (1.55 g, 22.8 mmol) at room temperature and the mixture was stirred at room temperature for 12 h. The reaction mixture was poured into water and the products were extracted with ethyl acetate. The extracts were washed with brine, dried over MgSO₄, filtered and concentrated in vacuo. The obtained residue was purified by silica gel chromatography (ethyl acetate/hexane=1/40) to give disilylated compound (1.4 g, 96%). To a stirred solution of the disilylated compound (1.33 g, 3.5 mmol) in MeOH (35 ml) was added PPTs (133 mg, 0.5 mmol) at room temperature and the mixture was stirred at room temperature for 17 h. The reaction mixture was poured into water and extracted with ethyl acetate. The extracts were washed with brine, dried over MgSO₄, filtered and concentrated in vacuo. The obtained residue was purified by silica gel chromatography (ethyl acetate/hexane=115) to give the titled compound (393 mg, 43%).

¹H-NMR (CDCl₃): 0.09 (3H, s), 0.11 (3H, s), 0.90 (9H, s), 1.25(3H, t), 1.92 (1H, dd), 2.55 (2H, d), 3.45-3.70 (2H, m),-4.12 (2H, q), 4.15-4.30 (1H, s).

### (2) Preparation of 3(S)-(tert-Butyl-dimethyl-silanyloxy)-4-(4-{1-ethyl-1-[3-methyl-4-(4,4,4-trifluoro-3-methoxymethoxy-3-trifluoromethyl-but-1-ynyl)-phenyl]-propyl}-2-methyl-phenoxy)-butyric acid ethyl ester

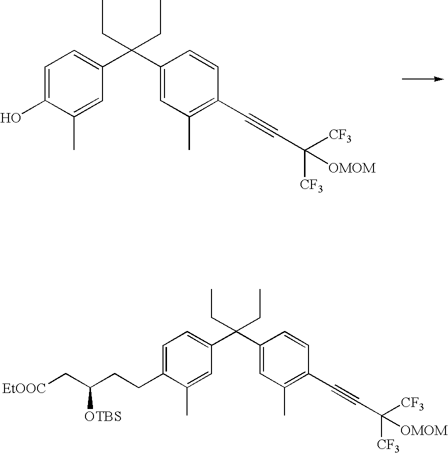

To a solution of 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) (255 mg, 0.502 mmol) and triphenylphosphine (145 mg, 0.552 mmol) in anhydrous Toluene (4 ml) was added dropwise DEAD (0.086 ml, 0.552 mmol) at 0 degrees C. under nitrogen atmosphere, and the mixture was stirred at room temperature. To the reaction mixture, a solution of 3-(tert-Butyl-dimethyl-silanyloxy)-4-hydroxy-butyric acid ethyl ester (compound prepared in Example 51-(1)) (158 mg, 0.602 mmol) in anhydrous Toluene (1 ml) was added. The resulting mixture was stirred at 60 degrees C. for 5 h. After removal of solvent, diethyl ether was added and the resulting mixture was stirred for 1 h. The insoluble solid was filtered off. The filtrate was concentrated in vacuo and the obtained residue was purified by silica gel chromatography (ethyl acetate/hexane=1/14) to give the titled compound (168 mg, 45%).

¹H-NMR (CDCl₃): 0.09 (s, 6H), 0.61 (t, 3H), 0.87 (s, 9H), 1.26 (t, 3H), 2.05 (q, 4H), 2.15 (s, 3H), 2.32 (s, 3H), 2.55 (dd, 1H), 2.70 (dd, 1H), 3.50 (s, 3H), 3.80 (dd, 1H), 3.96 (dd, 1H), 4.10˜4.19 (m, 2H), 4.48˜4.52 (m, 1H), 5.15 (s, 2H), 6.67 (d, 1H), 6.83 (s, 1H), 6.89 (dd, 1H), 7.00 (dd, 1H), 7.06 (s, 1H), 7.36 (d, 1H); MS (ESI+): 747([M+H]⁺).

### (3) Preparation of (S)-4-(4-{1-ethyl-1-[3-methyl-4-(4,4,4-trifluoro-3-hydroxy-3-trifluoromethyl-but-1-ynyl)-phenyl]-propyl}-2-methyl-phenoxy)-3-hydroxy-butyric acid

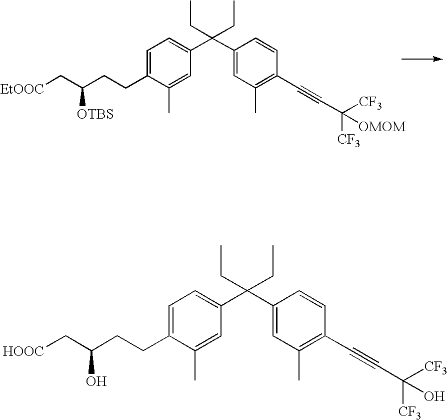

A stirred solution of 3(S)-(tert-Butyl-dimethyl-silanyloxy)4-(4-{1-ethyl-1-[3-methyl-4-(4,4,4-trifluoro-3-methoxymethoxy-3-trifluoromethyl-but-1-ynyl)-phenyl]-propyl}-2-methyl-phenoxy)-butyric acid ethyl ester (compound prepared in Example 51-(2)) (143 mg, 0.19 mmol) in 1,4-dioxane (4 ml), conc. HCl (0.2 ml) was slowly added at 0 degrees C. and the mixture was stirred at 50 degrees C. for 10 h. The reaction mixture was neutralized by careful addition of sat. NaHCO₃ solution and the products were extracted with ethyl acetate. The extracts were washed with brine, dried over MgSO₄, filtered and concentrated in vacuo. The obtained residue was dissolved in MeOH (5 ml). To the mixture, 1N KOH aq. (0.55 ml, 0.55 mmol) was added at room temperature and the mixture was stirred for 5 h. The reaction mixture was concentrated in vacuo and poured into saturated NH₄Cl aq. The products were extracted with ethyl acetate. The extracts were washed with brine, dried over MgSO₄, filtered and concentrated in vacuo. The obtained residue was chromatographed on silica gel (ethyl acetate/hexane=1/15) to give the titled compound (50 mg, 65%) as a white solid.

¹H-NMR (CDCl₃): 0.60 (t, 6H), 2.05 (q, 4H), 2.17 (s, 3H), 2.34 (s, 3H), 2.76 (t, 2H), 3.99 (d, 2H), 4.45 (m, 1H), 6.68 (d, 1H), 6.84 (s, 1H), 6.92 (dd, 1H), 6.97 (d, 1H), 7.04 (s, 1H), 7.34 (d, 1H); MS(ESI−): 559 ([M−H]⁻).

### Example 52

### Preparation of (S)4-(4-{1-ethyl-1-[3-methyl-4-((E)A4,4,4-trifluoro-3-hydroxy-3-trifluoromethyl-but-1-enyl)-phenyl]-propyl}-2-methyl-phenoxy)-3-hydroxy-butyric acid

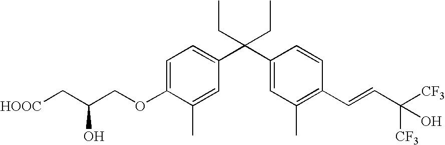

### (1) Preparation of (S)-3-(tert-Butyl-dimethyl-silanyloxy)-4-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxy)-butyric acid ethyl ester

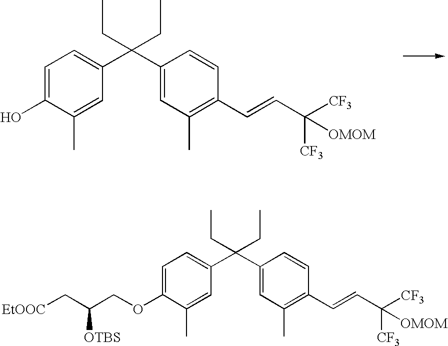

Using the same procedure as described for the preparation of Example 51-(2), the title compound was prepared from 4-{1-ethyl-1-[3-methyl4-((E)-4,4,4-trifluoro-3-methoxymethoxy-3-trifluoromethyl-but-1-enyl)-phenyl]-propyl}-2-methyl-phenol (compound prepared in Example 30-(1)).

¹H-NMR (CDCl₃): 0.09 (s, 6H), 0.61 (t, 3H), 0.87 (s, 9H), 1.26 (t, 3H), 2.05 (q, 4H), 2.15 (s, 3H), 2.32 (s, 3H), 2.55 (dd, 1H), 2.70 (dd, 1H), 3.50 (s, 3H), 3.80 (dd, 1H), 3.96 (dd, 1H), 4.10˜4.19 (m, 2H), 4.48˜4.52 (m, 1H), 4.96 (s, 2H), 6.05 (d, 1H), 6.68 (d, 1H), 6.87˜7.03 (m, 4H), 7.32 (d, 1H), 7.36 (s, 1H).

### (2) Preparation of (S)-4-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid

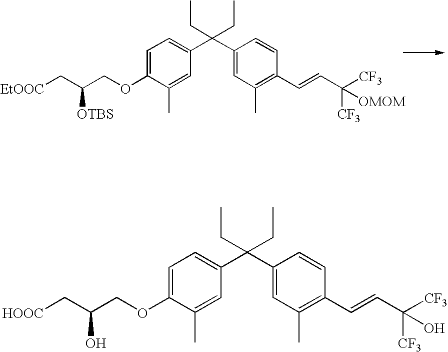

Using the same procedure as described for the preparation of Example 51-(3), the title compound was prepared from (S)-3-(tert-Butyl-dimethyl-silanyloxy)4-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxy)-butyric acid ethyl ester (compound prepared in Example 52-(1)).

¹H-NMR (CDCl₃): 0.61 (t, 6H), 2.05 (q, 4H), 2.17 (s, 3H), 2.34 (s, 3H), 2.76 (t, 2H), 3.99 (d, 2H), 4.45 (m, 1H), 6.09 (d, 1H), 6.69 (d, 1H), 6.90˜7.00 (m, 4H), 7.34 (t, 1H), 7.35 (d, 1H); MS (ESI−): 561 ([M−H]⁻).

### Example 53

### Preparation of (R)-4-(4-{1-ethyl-1-[3-methyl-4-(4,4,4-trifluoro-3-hydroxy-3-trifluoromethyl-but-1-ynyl)-phenyl]-propyl}-2-methyl-phenoxy)-3-hydroxy-butyric acid

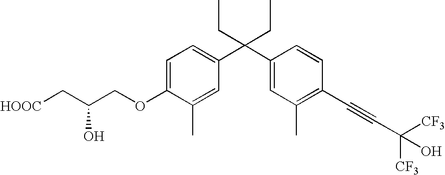

### (1) Preparation of 3(R)-(tert-butyl-dimethyl-silanyloxy)-4-hydroxy-butyric acid ethyl ester

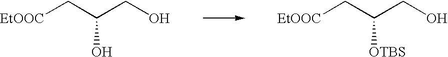

Using the same procedure as described for the preparation of Example 51-(1), the title compound was prepared from 3(S),4-dihydroxy-butyric acid ethyl ester (compound prepared using the same procedure in Tetrahedron, 1992, 48(20), 4067.) from diethyl (R)-malate).

¹H-NMR (CDCl₃): 0.09 (3H, s), 0.11 (3H, s), 0.89 (9H, s), 1.25(3H, t), 1.92 (1H, brt), 2.55 (2H, d), 3.45-3.70 (2H, m), 4.12 (2H, q), 4.15-4.30 (1H, s).

### (2) Preparation of (R)-3-(tert-Butyl-dimethyl-silanyloxy)-4-(4-{1-ethyl-1-[3-methyl-4-(4,4,4-trifluoro-3-methoxymethoxy-3-trifluoromethyl-but-1-ynyl)-phenyl]-propyl}-2-methyl-phenoxy)-butyric acid ethyl ester

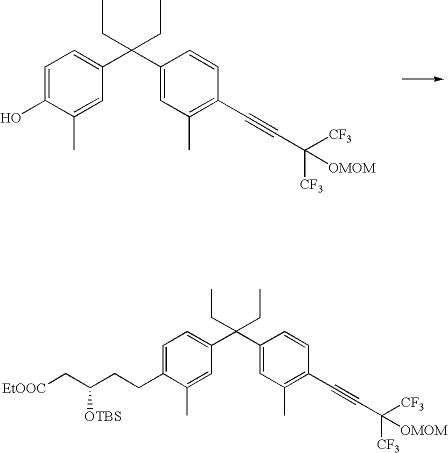

Using the same procedure as described for the preparation of Example 51-(2), the title compound was prepared from 4-{1-ethyl-1-[3-methyl4-(4,4,4-trifluoro-3-methoxymethoxy-3-trifluoromethyl-but-1-ynyl)-phenyl]-propyl}-2-methyl-phenol (compound prepared in Example 2-(2)) and 3(R)-(tert-Butyl-dimethyl-silanyloxy)-4-hydroxy-butyric acid ethyl ester (compound prepared in Example 53-(1)).

¹H-NMR (CDCl₃): 0.09 (s, 6H), 0.61 (t, 3H), 0.87 (s, 9H), 1.26 (t, 3H), 2.05 (q, 4H), 2.15 (s, 3H), 2.32 (s, 3H), 2.55 (dd, 1H), 2.70 (dd, 1H), 3.50 (s, 3H), 3.80 (dd, 1H), 3.96 (dd, 1H), 4.10˜4.19 (m, 2H), 4.48˜4.52 (m, 1H), 5.15 (s, 2H), 6.67 (d, 1H), 6.83 (s, 1H), 6.89 (dd, 1H), 7.00 (dd, 1H), 7.06 (s, 1H), 7.36 (d, 1H).

### (2) Preparation of (R)-4-(4-{1-ethyl-1-[3-methyl-4-(4,4,4-trifluoro-3-hydroxy-3-trifluoromethyl-but-1-ynyl)-phenyl]-propyl}-2-methyl-phenoxy)-3-hydroxy-butyric acid

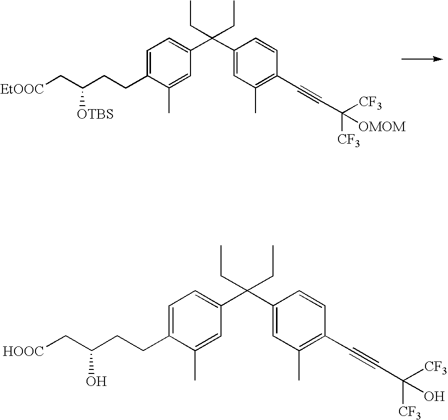

Using the same procedure as described for the preparation of Example 51-(3), the title compound was prepared from (R)-3-(tert-Butyl-dimethyl-silanyloxy)-4-(4-{1-ethyl-1-[3-methyl-4-(4,4,4-trifluoro-3-methoxymethoxy-3-trifluoromethyl-but-1-ynyl)-phenyl]-propyl}-2-methyl-phenoxy)-butyric acid ethyl ester (compound prepared in Example 53-(2)).

¹H-NMR (CDCl₃): 0.59 (t, 6H), 2.03 (q,.4H), 2.15 (s, 3H), 2.38 (s, 3H), 2.76 (t, 2H), 3.99 (d, 2H), 4.45 (m, 1H), 6.68 (d, 1H), 6.84 (d, 1H), 6.92 (dd, 1H), 6.97 (d, 1H), 7.04 (s, 1H), 7.34 (d, 1H); MS (ESI−): 559 ([M−H]⁻).

### Example 54

### Preparation of (R)-4-(4-{1-ethyl-1-[3-methyl4-((E)-4,4,4-trifluoro-3-hydroxy-3-trifluoromethyl-but-1-enyl)-phenyl]-propyl}-2-methyl-phenoxy)-3-hydroxy-butyric acid

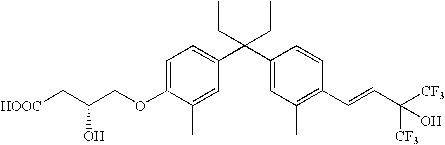

### (1) Preparation of (R)-3-(tert-Butyl-dimethyl-silanyloxy)4-(4-{1-ethyl-1-[3-methyl-4-((E)4,4,4-trifluoro-3-methoxymethoxy-3-trifluoromethyl-but-1-enyl)-phenyl]-propyl}-2-methyl-phenoxy)-butyric acid ethyl ester

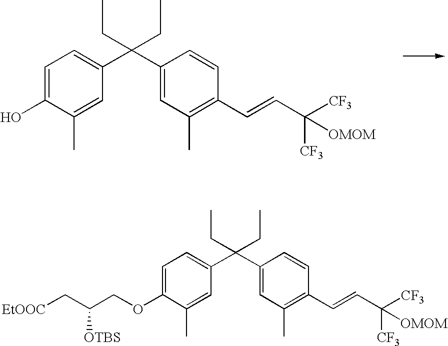

Using the same procedure as described for the preparation of Example 51-(2), the title compound was prepared from 4-{1-ethyl-1-[3-methyl4-((E)-4,4,4-trifluoro-3-methoxymethoxy-3-trifluoromethyl-but-1-enyl)-phenyl]-propyl}-2-methyl-phenol (compound prepared in Example 30-(1)) and 3R-(tert-Butyl-dimethyl-silanyloxy)4-hydroxy-butyric acid ethyl ester (compound prepared in Example 53-(1)).

¹H-NMR (CDCl₃): 0.09 (s, 6H), 0.61 (t, 3H), 0.87 (s, 9H), 1.26 (t, 3H), 2.05 (q, 4H), 2.15 (s, 3H), 2.32 (s, 3H), 2.55 (dd, 1H), 2.70 (dd, 1H), 3.50 (s, 3H), 3.80 (dd, 1H), 3.96 (dd, 1H), 4.10˜4.19 (m, 2H), 4.48˜4.52 (m, 1H), 4.96 (s, 2H), 6.05 (d, 1H), 6.68 (d, 1H), 6.87˜7.03 (m, 4H), 7.32 (d, 1H), 7.36 (s, 1H).

### (2) Preparation of (R)4-(4-{1-ethyl-1-[3-methyl4-((E)-4,4,4-trifluoro-3-hydroxy-3-trifluoromethyl-but-1-enyl)-phenyl]-propyl}-2-methyl-phenoxy)-3-hydroxy-butyric acid

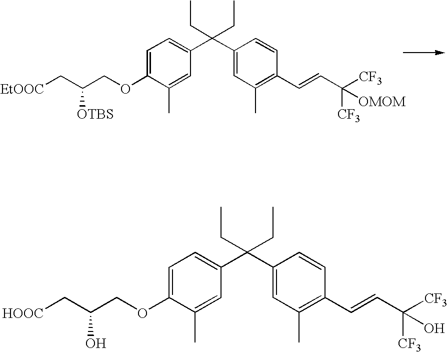

Using the same procedure as described for the preparation of Example 51-(3), the title compound was prepared from (R)-3-(tert-Butyl-dimethyl-silanyloxy)4-(4-{1-ethyl-1-[3-methyl4-((E)4,4,4-trifluoro-3-methoxymethoxy-3-trifluoromethyl-but-1-enyl)-phenyl]-propyl}-2-methyl-phenoxy)-butyric acid ethyl ester (compound prepared in Example 54-(1)).

¹H-NMR (CDCl₃): 0.60 (t, 6H), 2.04 (q, 4H), 2.17 (s, 3H), 2.34 (s, 3H), 2.76 (t, 2H), 3.99 (d, 2H), 4.46 (m, 1H), 6.08 (d, 1H), 6.69 (d, 1H), 6.90-7.00 (m, 4H), 7.26-7.39 (m, 2H); MS(ESI−): 561 ([M−H]⁻).

### Example 55

### Preparation of (S)-6-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one

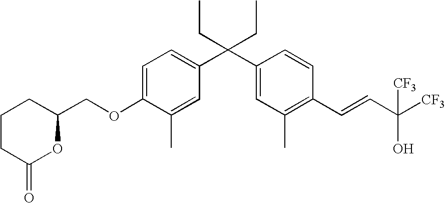

### Preparation of (S)-6-(4-{1-ethyl-I-[3-methyl-4-((E)4,4,4-trifluoro-3-hydroxy-3-trifluoromethyl-but-1-enyl)-phenyl]-propyl}-2-methyl-phenoxymethyl)-tetrahydro-pyran-2-one

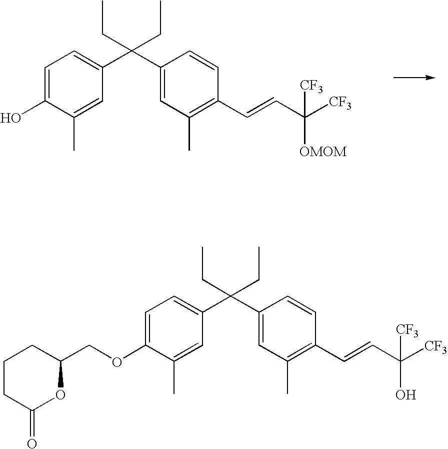

To a solution of 4-{1-ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in Example 30-(1)) (50 mg, 0.384 mmol) in toluene (3 ml) were added triphenylphosphine (151 mg, 0.576 mmol), DEAD (0.09 ml, 0.576 mmol) and (S)-6-Hydroxymethyl-tetrahydro-pyran-2-one (J. Chem. Soc., Perkin Trans. 1, 2000, 20, 3519) (194 mg, 0.384 mmol) and the mixture was stirred at room temperature overnight and at 40 degrees C. for 2 h. The mixture was filtered, and the filtrate was concentrated under reduced pressure. The obtained residue was purified by silica gel chromatography (EtOAc:Hex=1:5) to give (S)-6-(4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxymethyl)-tetrahydro-pyran-2-one (78 mg, 33%). To a solution of (S)-6-(4-{1-Ethyl-i-[3-methyl4-((E)4,4,4-trifluoro-3-methoxymethoxy-3-trifluoromethyl-but-1-enyl)-phenyl]-propyl}-2-methyl-phenoxymethyl)-tetrahydro-pyran-2-one (65 mg, 0.105 mmol) in CH₂Cl₂ (3 ml) at −30 degrees C. was added TMSBr (0.017 ml, 0.126 mmol) and the mixture was stirred at −30 degrees C. for 1 h and at 0 degrees C. for 30 min. The reaction mixture was poured into sat. NaHCO₃ aq. and the products were extracted with CH₂Cl₂. The extracts were dried over sodium sulfate, filtered and concentrated under reduced pressure. The obtained residue was purified by silica gel chromatography (EtOAc:Hex=1:4) to give the title compound (65 mg, 79%).

¹H-NMR (300 MHz, CDCl₃): 0.60 (t, 6H), 1.80-2.12 (m, 8H), 2.15 (s, 3H), 2.33 (s, 3H), 2.45-2.70 (m, 2H), 2.90 (t, 2H), 3.35 (s, 1H), 4.01-4.14 (m,2H), 4.66 (m, 1H), 6.07 (d, 1H), 6.67 (d, 1H), 6.87-7.01 (m, 4H), 7.32-7.39 (m, 2H); MS (ESI−): 571 ([M−H]⁻).

### Example 56

### Preparation of (S)-6-(4-{1-Ethyl-1-[3-methyl-4-((E)4,4,4-trifluoro-3-hydroxy-3-trifluoromethyl-but-1-enyl)-phenyl]-propyl}-2-methyl-phenoxy)-5-hydroxy-hexanoic acid

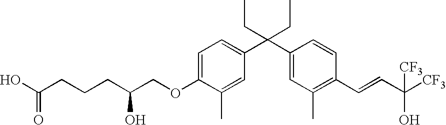

### Preparation of (S)-6-(4-{1-ethyl-1-[3-methyl4-((E)4,4,4-trifluoro-3-hydroxy-3-trifluoromethyl-but-1-enyl)-phenyl]-propyl}-2-methyl-phenoxy)-5-hydroxy-hexanoic acid

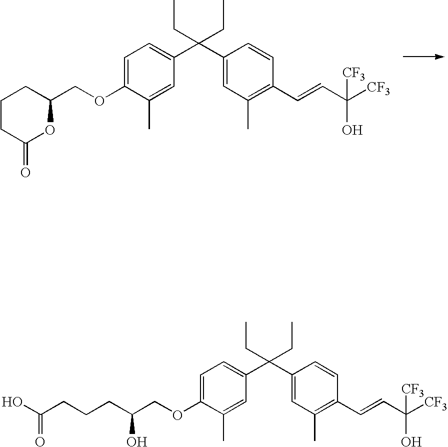

Using the same procedure as described for the preparation of Example 1-(7), the title compound was prepared from (S)-6-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one (compound prepared in Example 55). The yield was 91%.

¹H-NMR (300 MHz, CD₃OD as potassium salt): 0.63 (t, 6H), 1.61-1.84 (m, 4H), 2.10 (q, 4H), 2.18 (s, 3H), 2.24 (t, 2H), 2.35 (s, 3H), 2.45-2.70 (m, 2H), 2.90 (t, 2H), 3.35 (s, 1H), 3.90-4.03 (m, 3H), 6.21 (d, 1H), 6.79 (d, 1H), 6.89-7.03 (m, 4H), 7.37 (d, 1H), 7.44 (d, 1H); MS (ESI−): 589 ([M−H]⁻).

### Example 57

### Preparation of (S)-6-(4-{1-ethyl-1-[3-methyl4-(4,4,4-trifluoro-3-hydroxy-3-trifluoromethyl-but-1-ynyl)-phenyl]-propyl}-2-methyl-phenoxymethyl)-tetrahydro-pyran-2-one

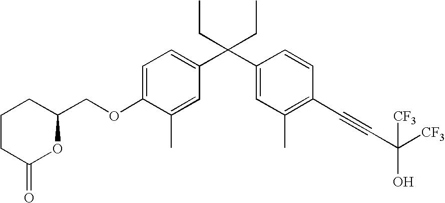

### (1) Preparation of (S)-6-(4-{1-ethyl-1-[3-methyl-4-(4,4,4-trifluoro-3-methoxymethoxy-3-trifluoromethyl-but-1-ynyl)-phenyl]-propyl}-2-methyl-phenoxymethyl)-tetrahydro-pyran-2-one

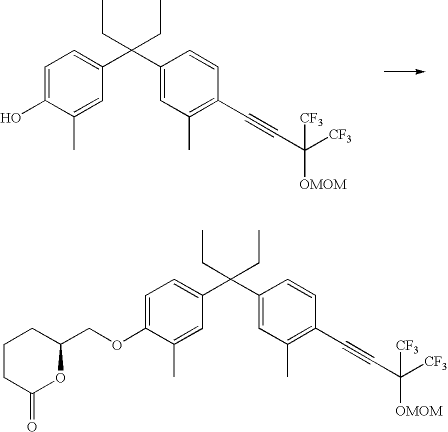

Using the same procedure as described for the preparation of Example 55, the title compound was prepared from 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) and (S)-6-hydroxymethyl-tetrahydro-pyran-2-one (J. Chem. Soc., Perkin Trans. 1, 2000, 20, 3519). The yield was 46%.

¹H-NMR (300 MHz, CDCl₃): 0.59 (t, 6H), 1.80-2.09 (m, 8H), 2.15 (s, 3H), 2.39 (s, 3H), 2.45-2.69 (m, 2H), 3.47 (s, 3H), 4.01-4.14 (m,2H), 4.69 (m, 1H), 5.15 (s, 2h), 6.67 (d, 1H), 6.84-7.04 (m, 4H), 7.36 (d, 1H).

### (2) Preparation of (S)-6-(4-{1-ethyl-1-[3-methyl4-(4,4,4-trifluoro-3-hydroxy-3-trifluoromethyl-but-1-ynyl)-phenyl]-propyl}-2-methyl-phenoxymethyl)-tetrahydro-pyran-2-one

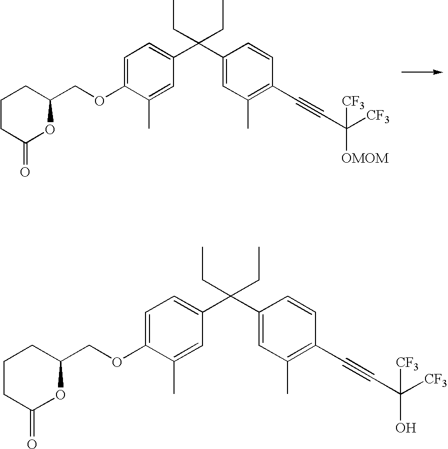

To a solution of (S)-6-(4-{1-ethyl-i-[3-methyl4-(4,4,4-trifluoro-3-methoxymethoxy-3-trifluoromethyl-but-1-ynyl)-phenyl]-propyl}-2-methyl-phenoxymethyl)-tetrahydro-pyran-2-one (compound prepared in Example 57-(1)) (78 mg, 0.127 mmol) in CH₂Cl₂ (2 ml) at 0 degrees C. were added thiophenol (0.020 ml, 0.190 mmol) and BF₃—OEt₂ (0.048 ml, 0.381 mmol) and the mixture was stirred at room temperature for 1 h. The reaction mixture was poured into phosphate buffer and the products were extracted with EtOAc. The extracts were dried over sodium sulfate, filtered and concentrated under reduced pressure. The obtained residue was purified by silica gel chromatography (EtOAc:Hex=1:5) to give the title compound (45 mg, 62%).

¹H-NMR (300 MHz, CDCl₃): 0.59 (t, 6H), 1.80-2.09 (m, 8H), 2.14 (s, 3H), 2.38 (s, 3H), 2.45-2.71 (m, 2H), 3.34 (s, 1H), 4.01-4.14 (m, 2H), 4.67 (m, 1H), 6.66 (d, 1H), 6.84-7.04 (m, 4H), 7.34 (d, 1H); MS (ESI+): 571 ([M+H]⁺).

### Example 58

### Preparation of (S)-6-(4-{1-Ethyl-1-[3-methyl4-(4,4,4-trifluoro-3-hydroxy-3-trifluoromethyl-but-1-ynyl)-phenyl]-propyl}-2-methyl-phenoxy)-5-hydroxy-hexanoic acid

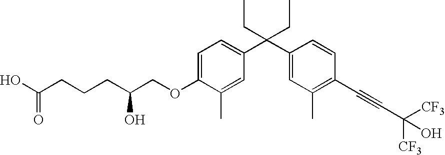

### Preparation of (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid

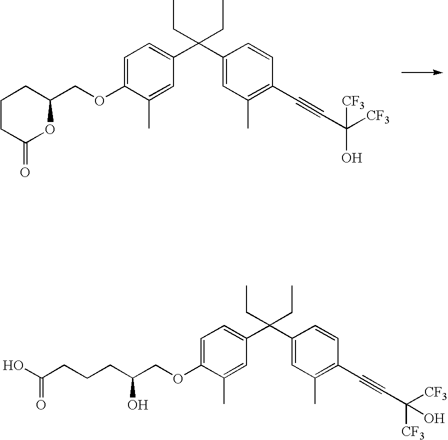

Using the same procedure as described for the preparation of Example 1-(7), the title compound was prepared from (S)-6-(4-{1-Ethyl-1-[3-methyl4-(4,4,4-trifluoro-3-hydroxy-3-trifluoromethyl-but-1-ynyl)-phenyl]-propyl}-2-methyl-phenoxymethyl)-tetrahydro-pyran-2-one (compound prepared in Example 57-(2)). The yield was 99%.

¹H-NMR (300 MHz, CD₃OD as potassium salt): 0.62 (t, 6H), 1.58-1.88 (m, 4H), 2.10 (q, 4H), 2.18 (s, 3H), 2.24 (t, 2H), 2.38 (s, 3H), 3.89-4.02 (m ,3H), 6.80 (d, 1H), 6.87-7.08 (m, 4H), 7.34 (d, 1H); MS (ESI−): 587 ([M−H]⁻).

### Example 59

### Preparation of (R)-6-(4-{1-Ethyl-1-[3-methyl4-(4,4,4-trifluoro-3-hydroxy-3-trifluoromethyl-but-1-ynyl)-phenyl]-propyl}-2-methyl-phenoxymethyl)-tetrahydro-pyran-2-one

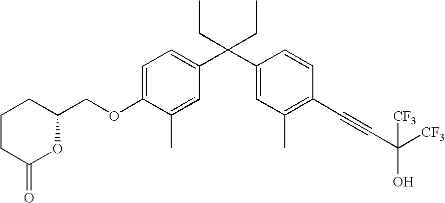

### (1) Preparation of (R)-6-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-tetrahydro-pyran-2-one

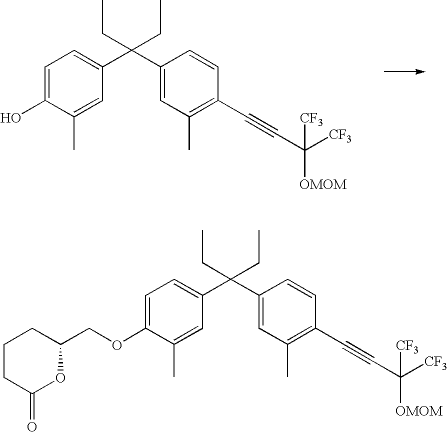

Using the same procedure as described for the preparation of Example 55, the title compound was prepared from 4-(1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) and (R)-6-hydroxymethyl-tetrahydro-pyran-2-one (J. Chem. Soc., Perkin Trans. 1, 2000, 20, 3519). The yield was 46%.

¹H-NMR(CDCl₃): 7.37(d, 1H), 7.04-6.84 (m, 4H), 6.67 (d, 1H), 5.15 (s, 2H), 4.69 (m, 1H), 4.12 (m, 2H), 3.84 (s, 3H), 2.60 (m, 2H), 2.39 (s, 3H), 2.15 (s, 3H), 2.04 (m, 6H), 1.88 (m, 2H), 0.59 (t, 6H).

### (2) Preparation of (R)-6-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one

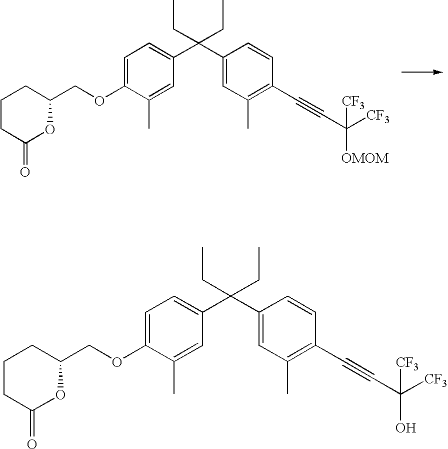

To a solution of (R)-6-(4-{1-ethyl-1-[3-methyl4-(4,4,4-trifluoro-3-methoxymethoxy-3-trifluoromethyl-but-1-ynyl)-phenyl]-propyl}-2-methyl-phenoxymethyl)-tetrahydro-pyran-2-one (compound prepared in Example 59-(1)) (43 mg, 0.07 mmol) in CH₂Cl₂ (3 ml) at −30 degrees C. was added TMSBr (0.014 ml, 0.105 mmol) and the mixture was stirred at −30 degrees C. for 1 h and at 0 degrees C. for 30 min. The reaction mixture was poured into sat. NaHCO₃ aq. and the products were extracted with CH₂Cl₂. The extracts were dried over sodium sulfate, filtered and concentrated under reduced pressure. The obtained residue was purified by silica gel chromatography (CH₂Cl₂: MeOH=30:1) to give the title compound (21 mg, 53%).

¹H-NMR(CDCl₃): 7.36 (d, 1H), 7.04 (s, 1H), 6.99 (s, 1H), 6.91 (dd, 1H), 6.84 (d, 1H), 6.66 (d, 1H), 4.69 (m, 1H), 4.09 (m, 2H), 2.58 (m, 2H), 2.38 (s, 3H), 2.15 (s, 3H), 2.04 (m, 6H), 1.88 (m, 2H), 0.59 (t, 6H); MS(ESI−): 569 ([M−H]⁻).

### Example 60

### Preparation of (R)-6-(4-{1-ethyl-1-[3-methyl4-(4,4,4-trifluoro-3-hydroxy-3-trifluoromethyl-but-1-ynyl)-phenyl]-propyl}-2-methyl-phenoxy)-5-hydroxy-hexanoic acid

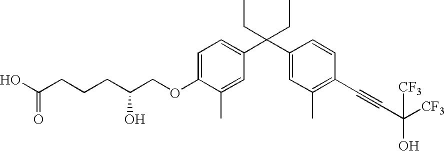

### Preparation of (R)-6-(4-{1-ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid

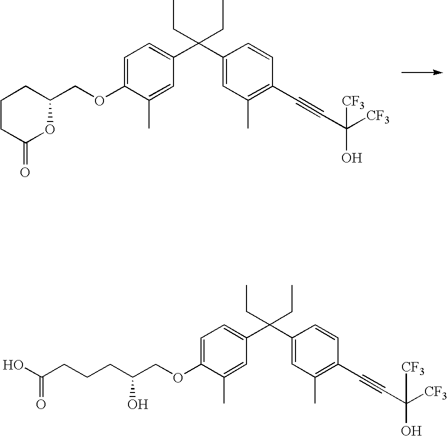

Using the same procedure as described for the preparation of Example 1-(7), the title compound was prepared from (R)-6-(4-{1-ethyl-1-[3-methyl4-(4,4,4-trifluoro-3-hydroxy-3-trifluoromethyl-but-1-ynyl)-phenyl]-propyl}-2-methyl-phenoxymethyl)-tetrahydro-pyran-2-one (compound prepared in Example 59-(2)). The yield was 99%.

¹H-NMR (300 MHz, CD₃OD as potassium salt): 0.62 (t, 6H), 1.58-1.84 (m, 4H), 2.10 (q, 4H), 2.18 (s, 3H), 2.24 (t, 2H), 2.38 (s, 3H), 3.89-3.99 (m,3H), 6.80 (d, 1H), 6.95-7.07 (m, 4H), 7.34 (d, 1H); MS (ESI−): 587 ([M−H]⁻).

### Example 61

### Preparation of (R)-6-(4-{1-ethyl-1-[3-methyl4-((E)4,4,4-trifluoro-3-hydroxy-3-trifluoromethyl-but-1-enyl)-phenyl]-propyl}-2-methyl-phenoxymethyl)-tetrahydro-pyran-2-one

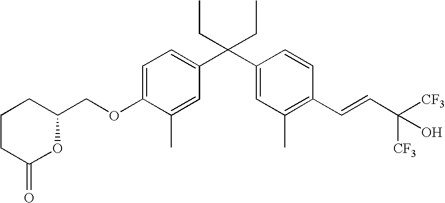

### (1) Preparation of (R)-6-(4-{1-ethyl-1-[3-methyl4-((E)4,4,4-trifluoro-3-methoxymethoxy-3-trifluoromethyl-but-1-enyl)-phenyl]-propyl}-2-methyl-phenoxymethyl)-tetrahydro-pyran-2-one

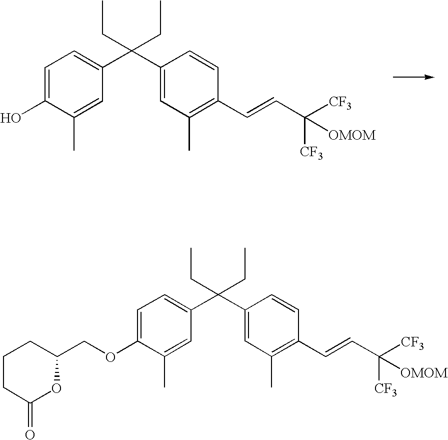

Using the same procedure as described for the preparation of Example 55, the title compound was prepared from 4-{1-ethyl-1-[3-methyl4-((E)4,4,4-trifluoro-3-methoxymethoxy-3-trifluoromethyl-but-1-enyl)-phenyl]-propyl}-2-methyl-phenol (compound prepared in Example 30-(1)) and (R)-6-hydroxymethyl-tetrahydro-pyran-2-one (J. Chem. Soc., Perkin Trans. 1, 2000, 20, 3519). The yield was 35%.

¹H-NMR (CDCl₃): 7.36 (d, 1H), 7.02-6.89 (m, 4H), 6.67 (d, 1H), 6.05 (d, 1H), 4.96 (s, 2H), 4.68 (m, 1H), 4.09 (m, 2H), 3.50 (s, 3H), 2.57 (m, 2H), 2.32 (s,3H), 2.16 (s, 3H), 2.05 (m, 6H), 1.89 (m, 2H), 0.61 (t, 6H).

### (2) Preparation of (R)-6-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one

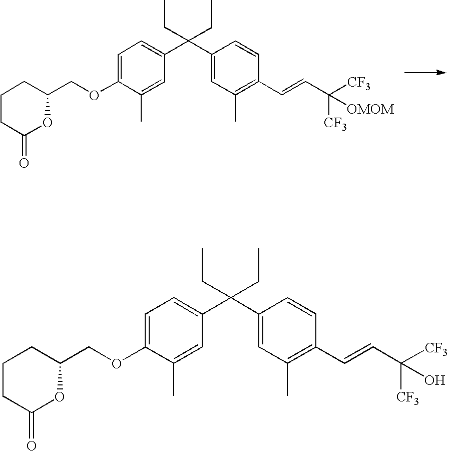

Using the same procedure as described for the preparation of Example 59-(2), the title compound was prepared from (R)-6-(4-{1-ethyl-1-[3-methyl-4-((E)4,4,4-trifluoro-3-methoxymethoxy-3-trifluoromethyl-but-1-enyl)-phenyl]-propyl}-2-methyl-phenoxymethyl)-tetrahydro-pyran-2-one (compound prepared in Example 61-(1)). The yield was 92%.

¹H-NMR(CDCl₃): 7.33 (d, 1H), 7.01-6.88 (m, 4H), 6.66 (d, ₁H), 6.07 (d, 1H), 4.67 (m, 1H), 4.08 (m, 2H), 2.58 (m, 2H), 2.33 (s,3H), 2.15 (s, 3H), 2.04 (t, 6H), 1.88 (m, 2H), 0.61 (t, 6H); MS(ESI−): 571.49 ([M−H]⁻).

### Example 62

### Preparation of (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid

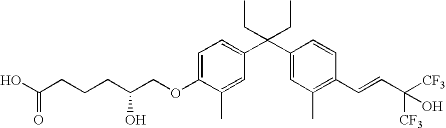

### Preparation of (R)-6-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid

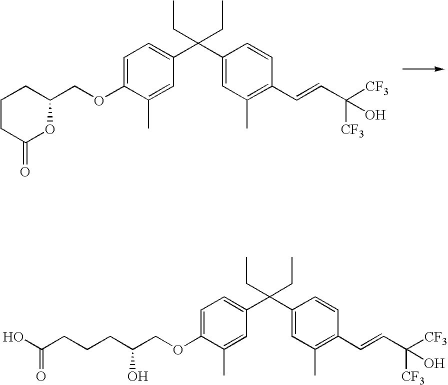

Using the same procedure as described for the preparation of Example 1-(7), the title compound was prepared from (R)-6-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one (compound prepared in Example 61-(2)). The yield was quant.

¹H-NMR (300 MHz, CD₃OD as potassium salt): 8.44 (d, 1H), 8.35 (d, 1H), 7.99 (d, 1H), 7.88 (d, 1H), 7.78 (d, 1H),7.21 (d, 1H), 4.92 (m, 3H), 3.35 (s, 3H), 3.24 (t, 2H), 3.17 (s, 3H), 3.10 (q, 4H), 2.91 (s, 3H), 2.73 (m, 2H), 2.61 (m, 1H), 1.6 (t, 6H); MS (ESI−): 589 ([M−H]⁻).

### Example 63

### Preparation of (S)-3-(4-{1-ethyl-1-[4-(3-ethyl-3-hydroxy-pent-1-ynyl)-3-methyl-phenyl]-propyl}-2-methyl-phenoxy)-propane-1,2-diol

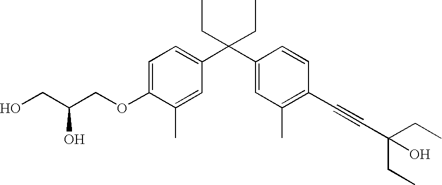

### (1) Preparation of 1-(4-{1-[4-((R)-2,2-dimethyl-[1,3]dioxolan-4-ylmethoxy)-3-methyl-phenyl]-1-ethyl-propyl}-2-methyl-phenyl)-3-ethyl-pent-1-yn-3-ol

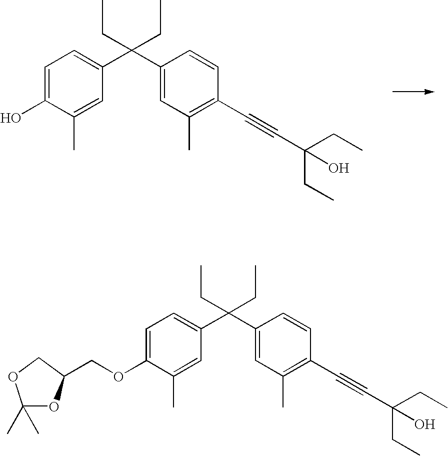

To a solution of 4-{1-ethyl-1-[4-(3-ethyl-3-hydroxy-pent-1-ynyl)-3-methyl-phenyl]-propyl}-2-methyl-phenol (compound prepared in Example 1-(4)) (250 mg, 0.660 mmol) in anhydrous DMF (6.6 ml) were added NaOH (40 mg, 0.99 mmol) and toluene-4-sulfonic acid (S)-2,2-dimethyl-[1,3]dioxolan-4-ylmethyl ester (214 mg, 0.792 mmol) and the mixture was stirred at 60 degrees C. for 11 h. The mixture was poured into saturated NH₄Cl aq. and the products were extracted with EtOAc. The extacts were washed with brine, dried over MgSO₄, filtered and concentrated under reduced pressure. The obtained residue was purified by Silica gel chromatography (EtOAc:n-Hexane=1:10) to give the title compound (177 mg, 56%) as a white solid.

¹H-NMR (300 MHz, CDCl₃): 0.59 (t, 6H), 1.11 (t, 6H), 1.40 (s, 3H), 1.46 (s, 3H), 1.70-1.83 (m, 4H), 2.04 (q, 4H), 2.14 (s, 3H), 2.37 (s, 3H), 3.89-3.99 (m, 2H), 4.04-4.19 (m, 3H), 4.42-4.50 (m, 1H), 6.68 (d, 1H), 6.84 (s, 1H), 6.90 (d, 2H), 6.99 (s, 1H), 7.27 (d, 1H).

### (2) Preparation of (S)-3-(4-{1-ethyl-1-[4-(3-ethyl-3-hydroxy-pent-1-ynyl)-3-methyl-phenyl]-propyl}-2-methyl-phenoxy)-propane-1,2-diol

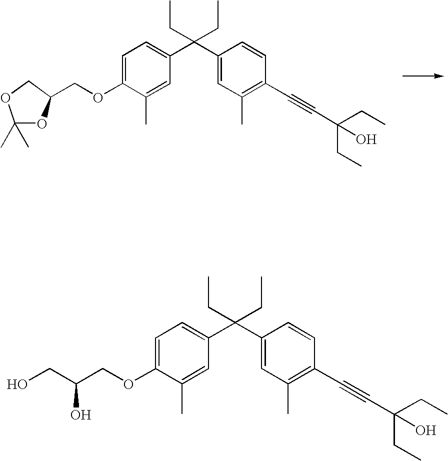

To a solution of 1-(4-{1-[4-((R)-2,2-dimethyl-[1,3]dioxolan4-ylmethoxy)-3-methyl-phenyl]-1-ethyl-propyl}-2-methyl-phenyl)-3-ethyl-pent-1-yn-3-ol (compound prepared in Example 63-(1)) (104 mg, 0.21.1 mmol) in THF:H₂O (4:1, 2.5 ml) was added TFA (1 ml, 12.98 mmol) and the mixture was stirred at 0 degrees C. for 3 h. The mixture was poured into saturated NaHCO₃ aq. and the products were extracted with EtOAc. The extacts were washed with brine, dried over MgSO₄, filtered and concentrated under reduced pressure. The obtained residue was purified by silica gel chromatography (EtOAc:n-Hexane=1:1) to give the title compound (63 mg, 66%).

¹H-NMR (300 MHz, CDCl₃): 0.59 (t, 6H), 1.11 (t, 6H), 1.70-1.81 (m, 4H), 2.00 (m, 1H), 2.04 (q, 4H), 2.07 (s, 3H), 2.37 (s, 3H), 2.52 (d, 1H), 3.82-3.90 (m, 2H), 4.03 (d, 2H), 4.11-4.17 (m, 1H), 6.69 (d, 1H), 6.86 (d, 1H), 6.90 (s, 1H), 6.94 (s, 1H), 6.99 (d, 1H), 7.27 (d, 1H); MS (ESI+): 470 ([M+H]⁺).

### Example 64

### Preparation of (S)-3-(4-{1-ethyl-1-[4-((E)-3-ethyl-3-hydroxy-pent-1-enyl)-3-methyl-phenyl]-propyl}-2-methyl-phenoxy)-propane-1,2-diol

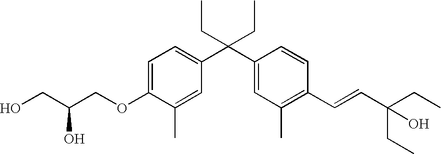

### (1) Preparation of (E)-1-(4-{1-[4-((R)-2,2-dimethyl-[1,3]dioxolan-4-ylmethoxy)-3-methyl-phenyl]-1-ethyl-propyl}-2-methyl-phenyl)-3-ethyl-pent-1-en-3-ol

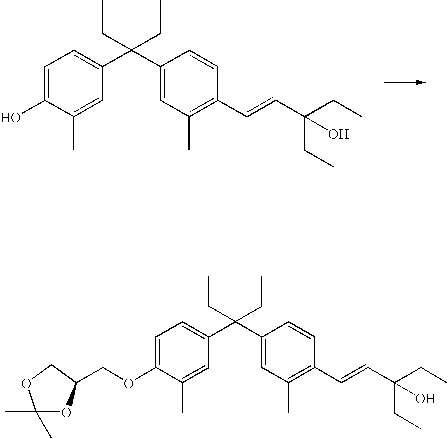

To a solution of 4-{1-ethyl-1-[4-((E)-3-ethyl-3-hydroxy-pent-1-enyl)-3-methyl-phenyl]-propyl}-2-methyl-phenol (compound prepared in Example 1-(5)) (150 mg, 0.394 mmol) in anhydrous DMF (6.6 ml) were added K₂CO₃ (136 mg, 0.985 mmol) and toluene-4-sulfonic acid (S)-2,2-dimethyl-[1,3]dioxolan-4-ylmethyl ester (169 mg, 0.591 mmol) and the mixture was stirred at 100 degrees C. for 14 h. The mixture was poured into saturated NH₄Cl aq. and the products were extracted with EtOAc. The extacts were washed with brine, dried over MgSO₄, filtered and concentrated under reduced pressure. The obtained residue was purified by silica gel chromatography (EtOAc:n-Hexane=1:7) to give the title compound (170 mg, 88%).

¹H-NMR (300 MHz, CDCl₃): 0.61 (t, 6H), 0.92 (t, 6H), 1.40 (s, 3H),1.46 (s, 3H), 1.63 (q, 4H), 2.04 (q, 4H), 2.16 (s, 3H), 2.31 (s, 3H), 3.89-4.16 (m, 4H), 4.48 (m, 1H), 6.00 (d, 1H), 6.68 (d, 1H), 6.74 (s, 1H), 6.91-6.97 (m, 4H), 7.29 (d, 1H).

### (2) Preparation of (S)-3-(4-{1-ethyl-1-[4-((E)-3-ethyl-3-hydroxy-pent-1-enyl)-3-methyl-phenyl]-propyl}-2-methyl-phenoxy)-propane-1,2-diol

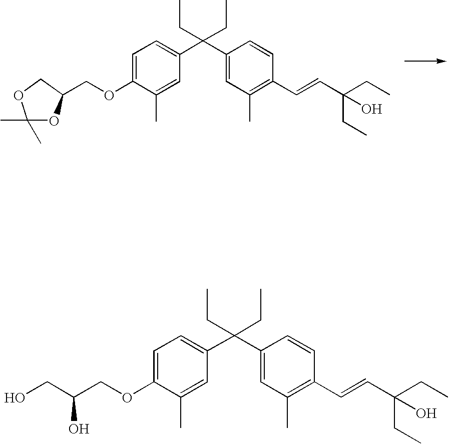

Using the same procedure as described for the preparation of Example 63-(2), the title compound was prepared from (E)-1-(4-{1-[4-((R)-2,2-dimethyl-[1,3]dioxolan-4-ylmethoxy)-3-methyl-phenyl]-1-ethyl-propyl}-2-methyl-phenyl)-3-ethyl-pent-1-en-3-ol (compound prepared in Example 64-(1)). The yield was 38%.

¹H-NMR (300 MHz, CDCl₃): 0.61 (t, 6H), 0.92 (t, 6H), 1.65 (q, 4H), 2.00 (m, 1H), 2.04 (q, 1H), 2.17 (s,3H), 2.31 (s,3H), 2.52 (d, 1H), 3.76-3.86 (m 2H), 4.03-4.04 (m, 2H), 4.08-4.14 (m, 1H), 6.01 (d, 1H), 6.72 (s, 1H), 6.73 (d, 1H), 6.91-6.98 (m, 4H), 7.29 (d, 1H).

### Example 65

### Preparation of (S)-3-(4-{1-Ethyl-1-[4-(3-ethyl-3-hydroxy-pentyl)-3-methyl-phenyl]-propyl}-2-methyl-phenoxy)-propane-1,2-diol

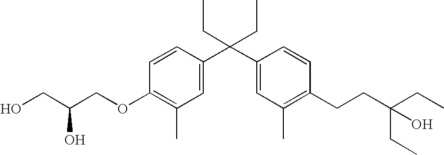

### (1) Preparation of 3-(4-{1-[4-((S)-2,3-dihydroxy-propoxy)-3-methyl-phenyl]-1-ethyl-propyl}-2-methyl-phenyl)-propionic acid methyl ester

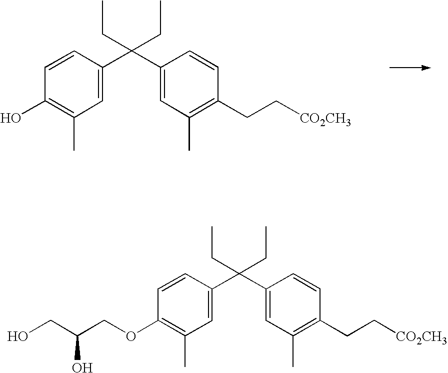

To a solution of 3-{4-[1-ethyl-1-(4-hydroxy-3-methyl-phenyl)-propyl]-2-methyl-phenyl}-propionic acid methyl ester (compound prepared in Example 18-(2)) (320 mg, 0.90 mmol) in anhydrous DMF (9 ml) at room temperature under nitrogen atmosphere were added K₂CO₃ (248 mg, 1.80 mmol) and (S)-toluene-4-sulfonic acid 2,2-dimethyl-[1,3]dioxolan-4-ylmethyl ester (309 mg, 1.08 mmol). After stirring overnight at 100 degrees C., the reaction mixture was diluted with ethyl acetate, washed with sat. NH₄Cl, water and brine, and dried over MgSO₄. The solution was concentrated under reduced pressure and the obtained residue was dissolved in methanol (9 ml). To the solution was added concentrated HCl with 4drops and the mixture was stirred at room temperature for 30 min. The mixture was concentrated under reduced pressure and the obtained residue was purified by silica-gel chromatography (hexane/EtOAc=7:1) to give the title compound (187 mg, 0.43 mmol, 48%).

¹H NMR (CDCl₃): 6.99-6.83 (m, 5H), 6.69 (d, 1H), 4.10 (m, 1H), 4.03 (dd, 2H), 3.82 (m, 2H), 3.67 (s, 3H), 2.89 (dt, 2H), 2.57 (dt, 2H), 2.25 (s, 3H), 2.16 (s, 3H), 2.04 (q, 4H), 0.59 (t, 6H).

### (2) Preparation of (S)-3-(4-{1-ethyl-1-[4-(3-ethyl-3-hydroxy-pentyl)-3-methyl-phenyl]-propyl}-2-methyl-phenoxy)-propane-1,2-diol

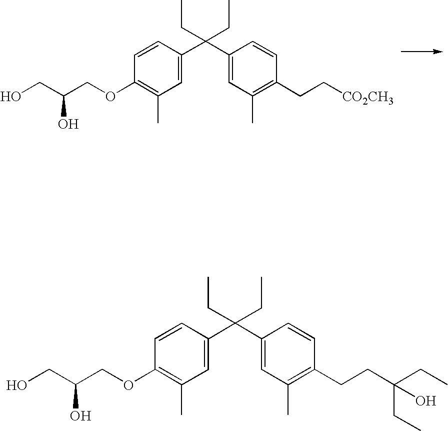

To a solution of 3-(4-{1-[4-((S)-2,3-dihydroxy-propoxy)-3-methyl-phenyl]-1-ethyl-propyl}-2-methyl-phenyl)-propionic acid methyl ester (compound prepared in Example 65-(1)) (187 mg, 0.43 mmol) in anhydrous THF (4 mL) under nitrogen atmosphere at 0 degrees C. was added ethyl magnesium bromide (1.3 ml, 1.3 mmol, 1.0 M in THF) and the mixture was stirred at 0 degrees C. for 2 h. The mixture was poured into saturated NH₄Cl aq. and the products were extracted with EtOAc. The extacts were washed with brine, dried over MgSO₄, filtered and concentrated under reduced pressure. The obtained residue was purified by silica gel chromatography (EtOAc:n-Hexane=1:7) to give the title compound (110 mg, 56%).

¹H NMR (CDCl₃): 7.01-6.89 (m, 5H), 6.69 (d, 1H), 4.10 (m, 1H), 4.02 (dd, 2H), 3.84 (dd, 1H), 3.76 (dd, 1H), 2.56 (m, 2H), 2.25 (s, 3H), 2.16 (s, 3H), 2.04 (q, 4H), 1.65 (m, 2H), 1.55 (q, 4H), 0.90 (t, 6H), 0.59 (t, 6H); MS (ESI−): 455 ([M−H]⁻).

### Example 66

### Preparation of 3-(4-{1-ethyl-1-[4-(3-hydroxy-4,4-dimethyl-pent-1-ynyl)-3-methyl-phenyl]-propyl}-2-methyl-phenoxy)-propane-1,2(S)-diol

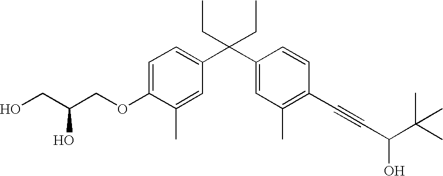

### (1) Preparation of 1-(4-{1-[4-((R)-2,2-dimethyl-[1,3]dioxolan4-ylmethoxy)-3-methyl-phenyl]-1-ethyl-propyl}-2-methyl-phenyl)-4,4-dimethyl-pent-1-yn-3-ol

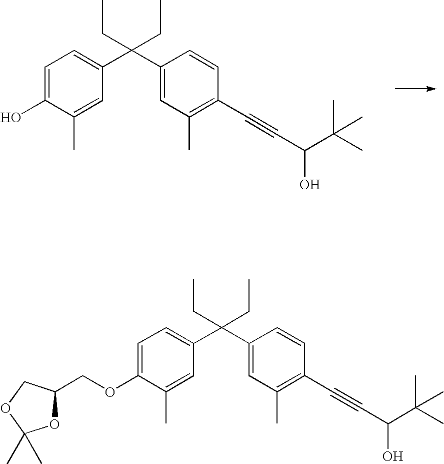

Using the same procedure as described for the preparation of Example 64-(1), the title compound was prepared from 4-{1-Ethyl-1-[4-(3-hydroxy-4,4-dimethyl-pent-1-ynyl)-3-methyl-phenyl]-propyl}-2-methyl-phenol (compound prepared in Example 3). The yield was 74%.

¹H-NMR (300 MHz, CDCl₃): 0.58 (t, 6H), 1.06 (s, 9H),1.40 (s, 3H), 1.45 (s, 3H), 1.76 (d, 1H), 2.03 (q, 4H), 2.14 (s, 3H), 2.37 (s, 3H), 3.89-3.98 (m, 2H), 4.03-4.16 (m, 2H), 4.25 (d, 1H), 4.42-4.50 (m, 1H), 6.68 (d, 1H), 6.85-6.99 (m, 4H), 7.28 (d, 1H).

### (2) Preparation of 3-(4-{1-ethyl-1-[4-(3-hydroxy-4,4-dimethyl-pent-1-ynyl)-3-methyl-phenyl]-propyl}-2-methyl-phenoxy)-propane-1,2(S)-diol

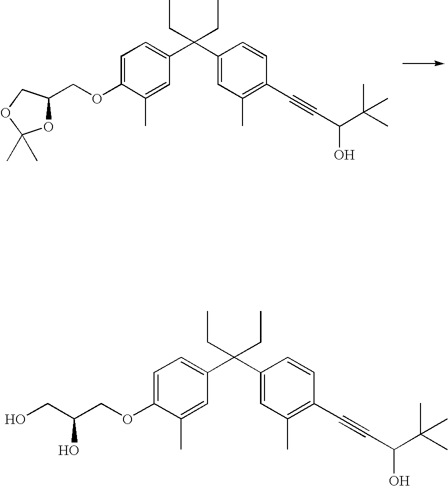

To a solution of 1-(4-{1-[4-((R)-2,2-dimethyl-[1,3]dioxolan-4-ylmethoxy)-3-methyl-phenyl]-1-ethyl-propyl}-2-methyl-phenyl)-4,4-dimethyl-pent-1-yn-3-ol (compound prepared in Example 66-(1)) (134 mg, 0.272 mmol) in MeOH (6 ml) was added conc.HCl (0.23 ml, 2.72 mmol) and the mixture was stirred at room temperature for 1 h. The reaction mixture was neutralized by sat. NaHCO₃ solution, extracted with CH₂Cl₂, dried over MgSO₄, filtered, and concentrated under reduced pressure. The obtained residue was purified by silica gel chromatography (EtOAc:hexane=1:1) to give the title compound (40 mg, 33%).

¹H-NMR (300 MHz, CDCl₃): 0.59 (t, 6H), 1.06 (s, 9H), 1.78 (d, 1H), 1.98-2.07 (m, 5H), 2.15 (s, 3H), 2.37 (s, 3H), 2.54 (d, 1H), 3.73-3.89 (m, 2H), 4.03 (m, 2H), 4.11 (m, 1H), 4.25 (d, 1H), 6.69 (d, 1H), 6.86-6.99 (m, 4H), 7.28 (d, 1H); MS (ESI+): 475 (M+Na)⁺.

### Example 67

### Preparation of 3-(4-{1-ethyl-1-[4-((E)-3-hydroxy-4,4-dimethyl-pent-1-enyl)-3-methyl-phenyl]-propyl}-2-methyl-phenoxy)-propane--1,2(S)-diol

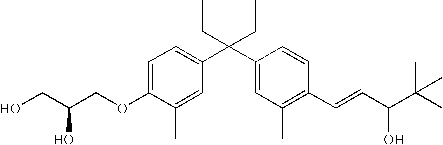

### (1) Preparation of (E)-1-(4-{1-[4-((R)-2,2-dimethyl-[1,3]dioxolan4-ylmethoxy)-3-methyl-phenyl]-1-ethyl-propyl}-2-methyl-phenyl)4,4-dimethyl-pent-1-en-3-ol

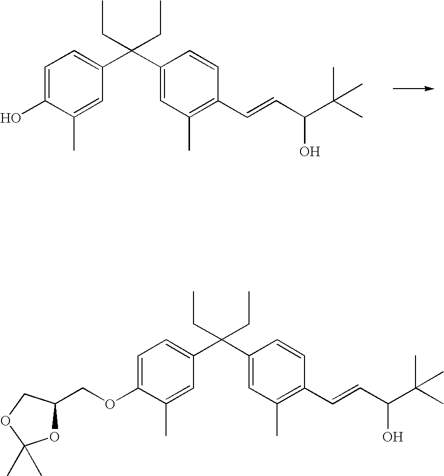

Using the same procedure as described for the preparation of Example 64-(1), the title compound was prepared from 4-{1-ethyl-1-[4-((E)-3-hydroxy-4,4-dimethyl-pent-1-enyl)-3-methyl-phenyl]-propyl}-2-methyl-phenol (compound prepared in Example 14). The yield was 74%.

¹H-NMR (300 MHz, CDCl₃): 0.60 (t, 6H), 0.96 (s, 9H), 1.40 (s, 3H), 1.45 (s, 3H), 2.04 (q, 4H), 2.15 (s, 3H), 2.29 (s, 3H), 3.89-3.98 (m, 3H), 4.04-4.08 (m, 1H), 4.13-4.18 (m, 1H), 4.42-4.49 (m, 1H), 6.12 (dd, 1H), 6.67-6.76 (m, 2H), 6.68-6.96 (m, 4H), 7.31 (d, 1H).

### (2) Preparation of 3-(4-{1-ethyl-1-[4-((E)-3-hydroxy-4,4-dimethyl-pent-1-enyl)-3-methyl-phenyl]-propyl}-2-methyl-phenoxy)-propane-1,2(S)-diol

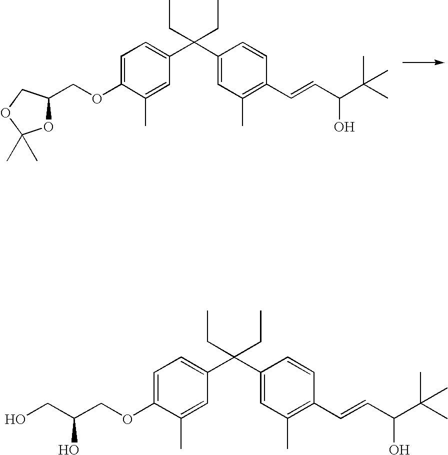

To a solution of (E)-1-(4-{1-[4-((R)-2,2-dimethyl-[1,3]dioxolan4-ylmethoxy)-3-methyl-phenyl]-1-ethyl-propyl}-2-methyl-phenyl)4,4-dimethyl-pent-1-en-3-ol (compound prepared in Example 67-(1)) (201 mg, 0.406 mmol) in THF/H₂O (10/1, 8 ml) was added 10-camphorsulfonic acid (289 mg, 1.21 mmol) and the mixture was stirred at 50 degrees C. for 1 h. The reaction mixture was poured into sat. NaHCO₃ solution, extracted with CH₂Cl₂, dried over MgSO₄, filtered, and concentrated under reduced pressure. The obtained residue was purified by ODS chromatography (MeOH/H₂O) to give the title compound (24.8 mg, 13%).

¹H-NMR (300 MHz, CDCl₃): 0.60 (t, 6H), 0.96 (s, 9H), 1.50 (m, 1H), 1.96-2.08 (m, 5H), 2.16 (s, 3H), 2.29 (s, 3H), 2.53 (d, 1H), 3.74-3.93 (m, 3H), 4.02-4.04 (m, 2H), 4.07-4.15 (m, 1H), 4.42-4.49 (m, 1H), 6.12 (dd, 1H), 6.68-6.76 (m, 2H), 6.90-6.98 (m, 4H), 7.30 (d, 1H); MS (ESI+): 477 ([M+Na]⁺).

### Example 68

### Preparation of 3-(4-{1-Ethyl-1-[3-methyl4-(4,4,4-trifluoro-3-hydroxy-3-trifluoromethyl-but-1-ynyl)-phenyl]-propyl}-2-methyl-phenoxy)-propane-1,2(S)-diol

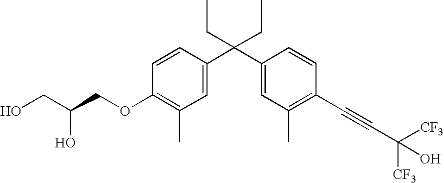

### (1) Preparation of (R)4-{4-[1-Ethyl-1-(4-ethynyl-3-methyl-phenyl)-propyl]-2-methyl-phenoxymethyl}-2,2-dimethyl-[1,3]dioxolane

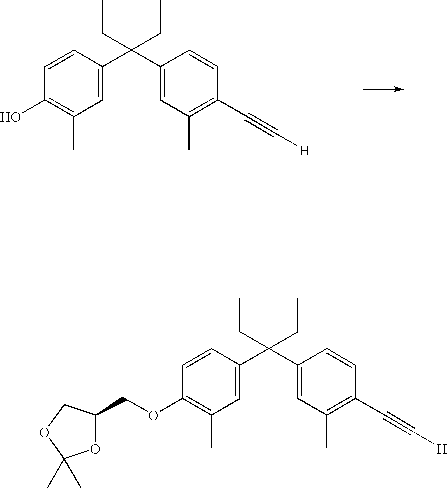

Using the same procedure as described for the preparation of Example 64-(1), the title compound was prepared from 4-[1-ethyl-1-(4-ethynyl-3-methyl-phenyl)-propyl]-2-methyl-phenol (compound prepared in Example 1-(3)). The yield was 43%.

¹H-NMR (300 MHz, CDCl₃): 7.33 (1H, d), 7.01 (1H, d), 6.95 (1H, d), 6.91 (1H, m), 6.86 (1H, d), 6.69 (1H, d), 4.46 (1H, m), 4.11 (2H, m), 3.94 (2H, m), 2.39 (3H, s), 2.15 (3H, s), 2.04 (4H, q), 1.46 (3H, s), 1.40 (3H, s), 0.59 (6H, t).

### (2) Preparation of 4-(4-{1-[4-((R)-2,2-dimethyl-[1,3]dioxolan-4-ylmethoxy)-3-methyl-phenyl]-1-ethyl-propyl}-2-methyl-phenyl)-1,1,1-trifluoro-2-trifluoromethyl-but-3-yn-2-ol

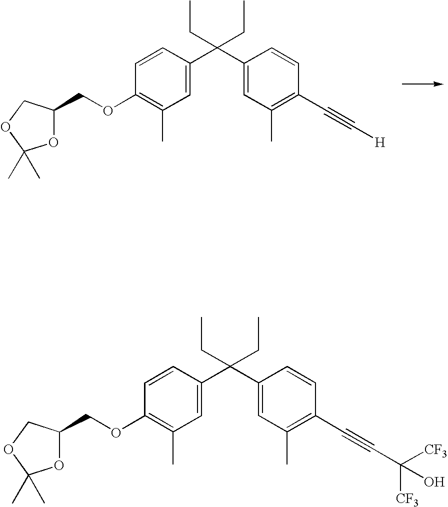

To a solution of (R)-4-{4-[1-ethyl-1-(4-ethynyl-3-methyl-phenyl)-propyl]-2-methyl-phenoxymethyl}-2,2-dimethyl-[1,3]dioxolane (compound prepared in Example 68-(1)) (540 mg, 1.328 mmol) in anhydrous THF (15 ml) at −78 degrees C. under nitrogen atmosphere was added n-BuLi (2.5N, 0.585 ml, 1.462 mmol) dropwise and the mixture was stirred for 20 min. Then to the mixture, hexafluoroacetone was added until the solution was made colorless. The reaction mixture was poured into saturated NH₄Cl aq. and the products were extracted with diethyl ether. The extacts were washed with brine, dried over MgSO₄, filtered and concentrated under reduced pressure. The obtained residue was purified by silica gel chromatography (EtOAc:n-Hexane=1:8) to give the title compound (686 mg, 90%).

¹H-NMR (300 MHz, CDCl₃): 7.32 (1H, d), 7.04 (1H, d), 6.96 (1H, dd), 6.90 (1H, dd), 6.82 (1H, d), 6.67 (1H, d), 4.46 (1H, m), 4.42 (1H, s), 4.10 (2H, m), 3.94 (2H, m), 2.37 (3H, s), 2.13 (3H, s), 2.04 (4H, q), 1.46 (3H, s), 1.40 (3H, s), 0.59 (6H, t).

### (3) Preparation of (S)-3-(4-{1-ethyl-1-[3-methyl4-(4,4,4-trifluoro-3-hydroxy-3-trifluoromethyl-but-1-ynyl)-phenyl]-propyl}-2-methyl-phenoxy)-propane-1,2-diol

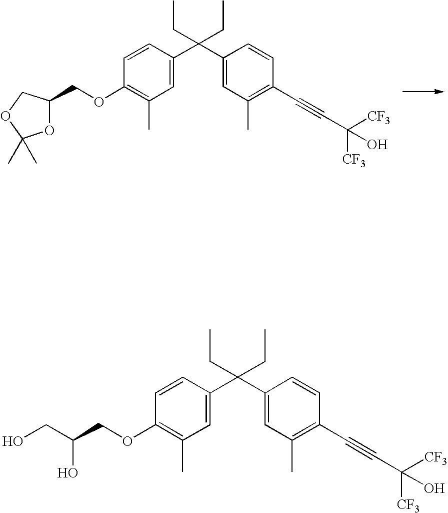

To a solution of 4-(4-{1-[4-((R)-2,2-dimethyl-[1,3]dioxolan-4-ylmethoxy)-3-methyl-phenyl]-1-ethyl-propyl}-2-methyl-phenyl)-1,1,1-trifluoro-2-trifluoromethyl-but-3-yn-2-ol (compound prepared in Example 68-(2)) (98 mg, 0.171 mmol) in THF (6 ml) was added 2N—HCl (2 ml) and the mixture was stirred at room temperature for 10 h. The reaction mixture was poured into water and the products were extracted with diethyl ether. The extracts were washed with brine and dried over MgSO₄, filtered and concentrated under reduced pressure. The obtained residue was purified by silica gel chromatography (n-Hexane:EtOAc=1:1) to give the title compound (83.5 mg, 92%).

¹H-NMR (300 MHz, CDCl₃): 7.34 (1H, d), 7.04 (1H, s), 6.96 (1H, dd), 6.92 (1H, dd), 6.83 (1H, d), 6.68 (1H, d), 4.11 (1H, m), 4.03 (1H, s), 4.02 (2H, d), 3.82 (2H, m), 2.61 (1H, d), 2.37 (3H, s), 2.14(3H, s), 2.04 (4H, q), 1.64 (1H, s), 0.60 (6H, t); MS (ES−): 531.08 ([M−H]⁻).

### Example 69

### Preparation of (S)-3-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol

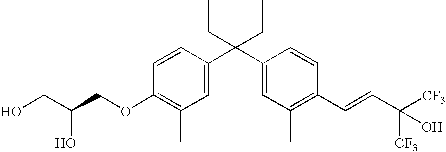

### (1) Preparation of (E)4-(4-{1-[4-((R)-2,2-dimethyl-[1,3]dioxolan4-ylmethoxy)-3-methyl-phenyl]-1-ethyl-propyl}-2-methyl-phenyl)-1,1,1-trifluoro-2-trifluoromethyl-but-3-en-2-ol

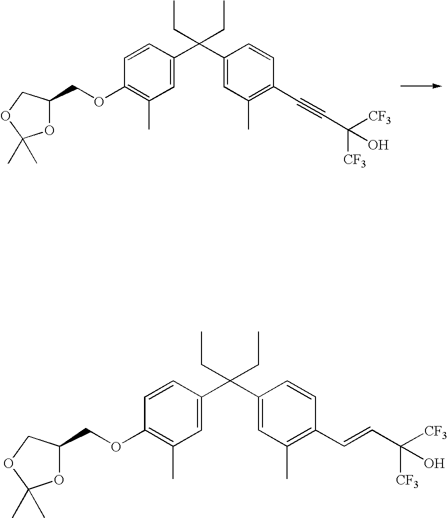

To a solution of 4-(4-{1-[4-((R)-2,2-dimethyl-[1,3]dioxolan4-ylmethoxy)-3-methyl-phenyl]-1-ethyl-propyl}-2-methyl-phenyl)-1,1,1-trifluoro-2-trifluoromethyl-but-3-yn-2-ol (compound prepared in Example 68-(2)) (168 mg, 0.293 mmol) in anhydrous THF (5 ml) was added LiAlH₄ (0.350 ml, 0.350 mmol) at room temperature under N₂ atmosphere and the mixture was stirred for 1 h and refluxed for 1 h. The reaction mixture was poured into saturated NH₄Cl aq. and the products were extracted with diethyl ether. The extacts were washed with brine, dried over MgSO₄, filtered and concentrated under reduced pressure. The obtained residue was purified by silica gel chromatography (EtOAc:n-Hexane=1:8) to give the title compound (154 mg, 91%).

¹H-NMR (300 MHz, CDCl₃): 7.37 (1 H, d), 7.33 (1H, d), 7.01 (1H, d), 6.94 (1H, s), 6.91 (1H, m), 6.88 (1H, d), 6.68 (1H, d), 6.10 (1H, d), 4.46 (1H, m), 4.11 (2H, m), 3.94 (2H, m), 3.33 (1H, s), 2.33 (3H, s),.2.16 (3H, s), 2.05 (4H, q), 1.45 (3H, s), 1.40 (3H, s), 0.60 (6H, t).

### (2) Preparation of (S)-3-(4-{1-ethyl-1-[3-methyl4-((E)4,4,4-trifluoro-3-hydroxy-3-trifluoromethyl-but-1-enyl)-phenyl]-propyl}-2-methyl-phenoxy)-propane-1,2-diol

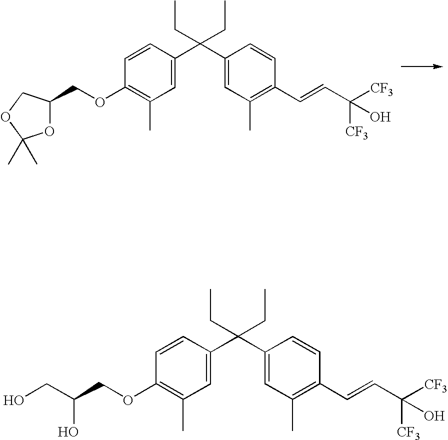

Using the same procedure as described for the preparation of Example 68-(3), the title compound was prepared from (E)4-(4-{1-[4-((R)-2,2-dimethyl-[1,3]dioxolan4-ylmethoxy)-3-methyl-phenyl]-1-ethyl-propyl}-2-methyl-phenyl)-1,1,1-trifluoro-2-trifluoromethyl-but-3-en-2-ol (compound prepared in Example 69-(1)). The yield was 74%.

¹H-NMR (300 MHz, CDCl₃): 7.37 (1H, d), 7.33 (1H, d), 7.00 (1H, d), 6.98 (1H, s), 6.94 (1H, dd), 6.89 (1H, d), 6.70 (1H, d), 6.08 (1H, d), 4.11 (1H, m), 4.04 (1H, s), 4.02 (1H, d), 3.81 (2H, m), 3.36 (1H, s), 2.57 (1H, d), 2.33 (3H, s), 2.16 (3H, s), 2.05(4H, q), 2.04(1H, s), 0.60 (6H, t); MS (ES−): 533 ([M−H]⁻).

### Example 70

### Preparation of (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol

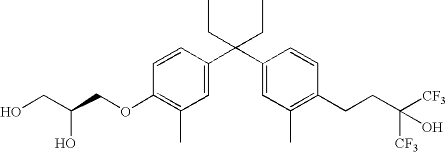

### (1) Preparation of 4-(4-{1-[4-((R)-2,2-dimethyl-[1,3]dioxolan-4-ylmethoxy)-3-methyl-phenyl]-1-ethyl-propyl}-2-methyl-phenyl)-1,1,1-trifluoro-2-trifluoromethyl-butan-2-ol

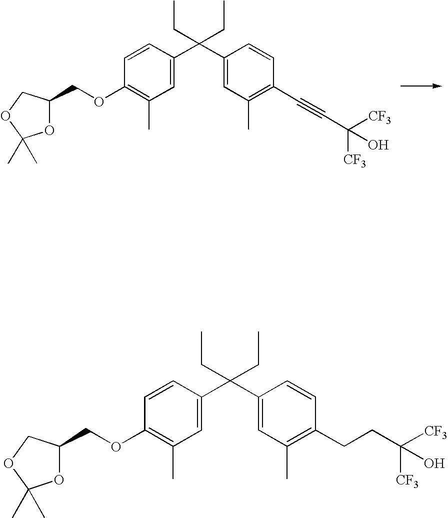

To a solution of 4-(4-{1-[4-((R)-2,2-dimethyl-[1,3]dioxolan-4-ylmethoxy)-3-methyl-phenyl]-1-ethyl-propyl}-2-methyl-phenyl)-1,1,1-trifluoro-2-trifluoromethyl-but-3-yn-2-ol (compound prepared in Example 68-(2)) (110 mg, 0.192 mmol) in MeOH (5 ml) was added Pd—C (20 mg, 10 wt %). The mixture was stirred at room temperature for 20 h under H₂ atmosphere. The mixture was filtered through celite 545, and the filtrate was concentrated under reduced pressure. The obtained residue was purified by silica gel chromatography (EtOAc:n-Hex=1:7) to give the title compound (106 mg, 96%).

¹H-NMR (300 MHz, CDCl₃): 7.02-6.88 (5H, m), 6.68 (1H, d), 4.46 (1H, m), 4.16 (1H, dd), 4.06 (1H, dd), 3.94 (2H, m), 3.12 (1H, s), 2.80 (2H, dd), 2.25 (3H, s), 2.16 (3H, s), 2.15 (2H, m), 2.03 (4H, q), 1.46 (3H, s), 1.40 (3H, s), 0.59 (6H, t).

### (2) Preparation of (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol

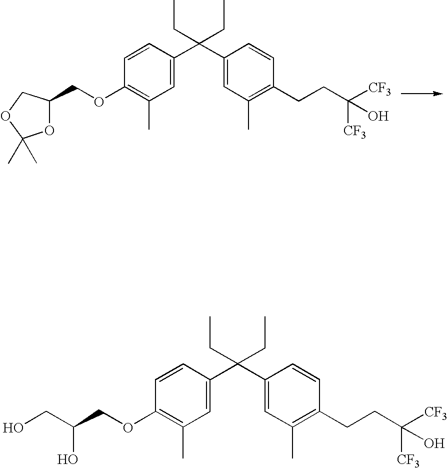

Using the same procedure as described for the preparation of Example 68-(3), the title compound was prepared from 4-(4-{1-[4-((R)-2,2-dimethyl-[1,3]dioxolan-4-ylmethoxy)-3-methyl-phenyl]-1-ethyl-propyl}-2-methyl-phenyl)-1,1,1-trifluoro-2-trifluoromethyl-butan-2-ol (compound prepared in Example 70-(1)). The yield was 73%.

¹H-NMR (300 MHz, CDCl₃): 7.02-6.88 (5H, m), 6.70 (1H, d), 4.11 (1H, m), 4.04 (1H, s), 4.03 (1H, s), 3.82 (2H, m), 3.43 (1H, brd), 2.80 (2H, dd), 2.65 (1H, brd), 2.25 (3H, s), 2.16 (3H, s), 2.15 (2H, m), 2.03 (4H, q), 0.59 (6H, t); MS (ES−): 535 ([M−H]⁻).

### Example 71

### Preparation of 2-(4-{1-ethyl-1-[4-(3-ethyl-3-hydroxy-pent-1-ynyl)-3-methyl-phenyl]-propyl}-2-methyl-phenoxymethyl)-propane-1,3-diol

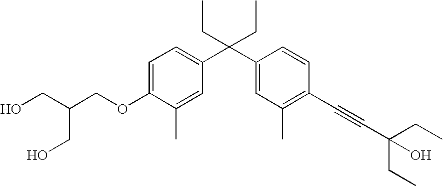

### (1) Preparation of 1-(4-{1-[4-(2,2-dimethyl-[1,3]dioxan-5-ylmethoxy)-3-methyl-phenyl]-1-ethyl-propyl}-2-methyl-phenyl)-3-ethyl-pent-1-yn-3-ol

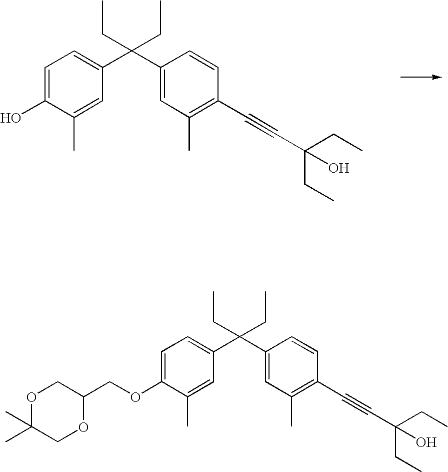

To a solution of 4-{1-ethyl-1-[4-(3-ethyl-3-hydroxy-pent-1-ynyl)-3-methyl-phenyl]-propyl}-2-methyl-phenol (compound prepared in Example 1-(4)) (205 mg, 0.542 mmol) in anhydrous DMF (5.4 ml) were added K₂CO₃ (187 mg, 1.36 mmol) and toluene-4-sulfonic acid 2,2-dimethyl-[1,3]dioxan-5-ylmethyl ester (prepared from following procedure as described in Tetrahedron, 1991, 47, 1001.) (244 mg, 1.355 mmol) and the mixture was stirred at 110 degrees C. for 15 h. The reaction mixture was poured into saturated NH₄Cl aq. and the products were extracted with EtOAc. The extacts were washed with brine, dried over MgSO₄, filtered and concentrated under reduced pressure. The obtained residue was purified by silica gel chromatography (EtOAc:n-Hexane=1:6) to give the title compound (184 mg, 67%).

¹H-NMR (300 MHz, CDCl₃): 0.59 (t, 6H), 1.11 (t, 6H), 1.43 (s, 3H), 1.46 (s, 3H), 1.72-1.85 (m, 4H), 2.04 (q, 4H), 2.14 (s, 3H), 2.13-2.20 (m, 1H), 2.37 (s, 3H), 3.89-4.20 (m, 5H), 6.68 (d, 1H), 6.84 (s, 1H), 6.91-6.94 (m, 2H), 6.99 (s, 1H), 7.27 (d, 1H).

### (2) Preparation of 2-(4-{1-Ethyl-1-[4-(3-ethyl-3-hydroxy-pent-1-ynyl)-3-methyl-phenyl]-propyl}-2-methyl-phenoxymethyl)-propane-1,3-diol

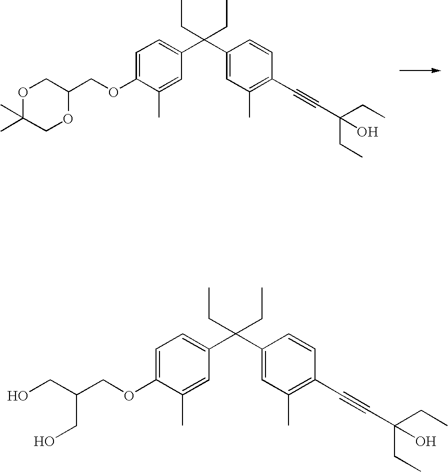

Using the same procedure as described for the preparation of Example 63-(2), the title compound was prepared from 1-(4-{1-[4-(2,2-Dimethyl-[1,3]dioxan-5-ylmethoxy)-3-methyl-phenyl]-1-ethyl-propyl}-2-methyl-phenyl)-3-ethyl-pent-1-yn-3-ol (compound prepared in Example 71-(1)). The yield was 38%.

¹H-NMR (300 MHz, CDCl₃): 0.61 (t, 6H), 0.92 (t, 6H), 1.57-1.70 (m, 4H), 2.04 (q, 4H), 2.15 (s,3H), 2.26 (t, 3H), 2.31 (s,3H), 3.95 (d, 4H), 4.09 (d, 2H), 6.70 (d, 1H), 6.82 (s, 1H), 6.88 (d, 2H), 6.98 (s, 1H), 7.27 (d, 1H); MS (ESI−): 465 ([M−H]⁻).

### Example 72

### Preparation of 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol

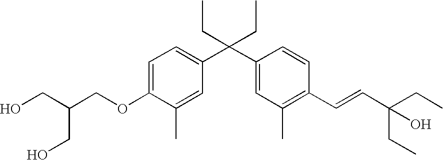

### (1) Preparation of (E)-1-(4-{1-[4-(2,2-dimethyl-[1,3]dioxan-5-ylmethoxy)-3-methyl-phenyl]-1-ethyl-propyl}-2-methyl-phenyl)-3-ethyl-pent-1-en-3-ol

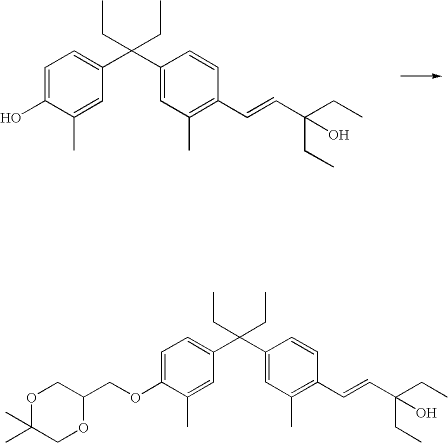

Using the same procedure as described for the preparation of Example 71-(1), the title compound was prepared from 4-{1-ethyl-1-[4-((E)-3-ethyl-3-hydroxy-pent-1-enyl)-3-methyl-phenyl]-propyl}-2-methyl-phenol (compound prepared in Example 1-(5)). The yield was 85%.

¹H-NMR (300 MHz, CDCl₃): 0.61 (t, 6H), 0.92 (t, 6H), 1.43 s, 3H), 1.46 (s, 3H), 1.64 (q, 4H), 2.04 (q, 4H), 2.15 (s, 3H), 2.18 (m, 1H), 2.31 (s, 3H), 3.89-4.20 (m, 6H), 6.01 (d, 1H), 6.68 (d, 1H), 6.74 (d, 1H), 6.90-7.02 (m, 4H), 7.29 (d, 1H).

### (2) Preparation of 2-(4-{1-ethyl-1-[4-((E)-3-ethyl-3-hydroxy-pent-1-enyl)-3-methyl-phenyl]-propyl}-2-methyl-phenoxymethyl)-propane-1,3-diol

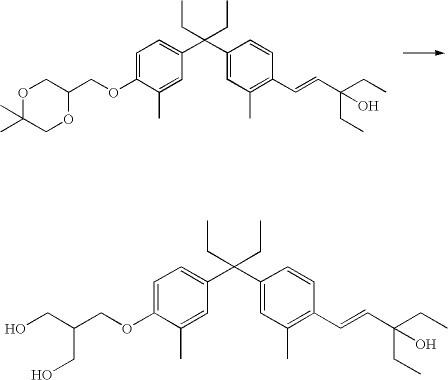

Using the same procedure as described for the preparation of Example 63-(2), the title compound was prepared from (E)-1-(4-{1-[4-(2,2-dimethyl-[1,3]dioxan-5-ylmethoxy)-3-methyl-phenyl]-1-ethyl-propyl}-2-methyl-phenyl)-3-ethyl-pent-1-en-3-ol (compound prepared in Example 72-(1)). The yield was 44%.

¹H-NMR (300 MHz, CDCl₃): 0.61 (t, 6H), 0.92 (t, 6H), 1.57-1.70 (m, 6H), 2.04 (q, 4H), 2.15 (s,3H), 2.26 (m, 3H), 2.31 (s,3H), 3.95 (d, 4H), 4.09 (d, 2H), 6.01 (d, 1H), 6.71 (d, 1H), 6.74 (s, 1H), 6.89 (d, 1H), 6.96-7.02 (m, 3H), 7.32 (d, 1H); MS (ESI−): 467 ([M−H]⁻).

### Example 73

### Preparation of 2-(4-{1-ethyl-1-[4-(3-ethyl-3-hydroxy-pentyl)-3-methyl-phenyl]-propyl}-2-methyl-phenoxymethyl)-propane-1,3-diol

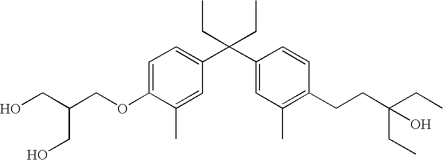

### (1) Preparation of 1-(4-{1-[4-(2,2-dimethyl-[1,3]dioxan-5-ylmethoxy)-3-methyl-phenyl]-1-ethyl-propyl}-2-methyl-phenyl)-3-ethyl-pentan-3-ol

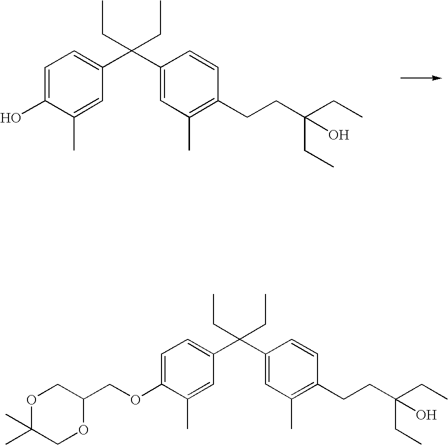

Using the same procedure as described for the preparation of Example 71-(1), the title compound was prepared from 4-{1-ethyl-1-[4-(3-ethyl-3-hydroxy-pentyl)-3-methyl-phenyl]-propyl}-2-methyl-phenol (compound prepared by hydrogenation of 4-{1-ethyl-1-[4-(3-ethyl-3-hydroxy-pent-1-ynyl)-3-methyl-phenyl]-propyl}-2-methyl-phenol (Example 1-(4)). The yield was 73%.

¹H-NMR(300 MHz, CDCl₃): 0.60 (t, 6H), 0.91 (t, 6H), 1.43 (s, 3H), 1.46 (s, 3H), 1.55 (m, 6H), 1.63-1.69 (m, 1H), 2.02 (q, 4H), 2.14 (s, 3H), 2.18 (m, 1H), 2.26 (s, 3H), 2.55 (m, 2H), 3.89-4.16 (m, 6H), 6.68 (d, 1H), 6.90-6.97 (m, 4H), 7.00 (d, 1H); MS (ESI+): 528 ([M+NH₄]⁺)

### (2) Preparation of 2-(4-{1-ethyl-1-[4-(3-ethyl-3-hydroxy-pentyl)-3-methyl-phenyl]-propyl}-2-methyl-phenoxymethyl)-propane-1,3-diol

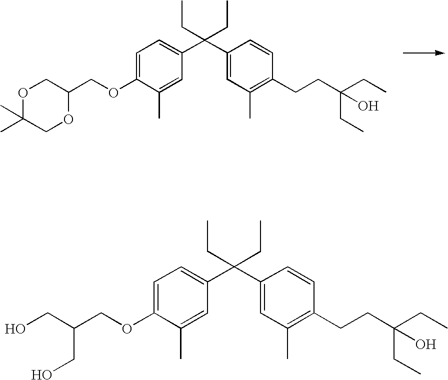

Using the same procedure as described for the preparation of Example 63-(2), the title compound was prepared from 1-(4-{1-[4-(2,2-Dimethyl-[1,3]dioxan-5-ylmethoxy)-3-methyl-phenyl]-1-ethyl-propyl}-2-methyl-phenyl)-3-ethyl-pentan-3-ol (compound prepared in Example 73-(1)). The yield was 73%.

¹H-NMR (300 MHz, CDCl₃): 0.59 (t, 6H), 0.89 (t, 6H), 1.54 (q, 4H), 1.65 (m, 2H), 2.00 (q, 4H), 2.15 (s,3H), 2.25 (m, 1H), 2.31 (s,3H), 2.53-2.59 (m, 2H), 3.93 (d, 4H), 4.07 (d, 2H), 6.70 (d, 1H), 6.91-7.00 (m, 5H); MS (ESI−): 469 ([M−H]⁻).

### Example 74

### Preparation of 2-(4-{1-ethyl-1-[4-(3-hydroxy-4,4-dimethyl-pent-1-ynyl)-3-methyl-phenyl]-propyl}-2-methyl-phenoxymethyl)-propane-1,3-diol

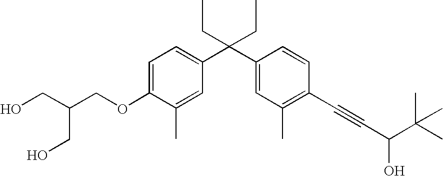

### (1) Preparation of 1-(4-{1-[4-(2,2-dimethyl-[1,3]dioxan-5-ylmethoxy)-3-methyl-phenyl]-1-ethyl-propyl}-2-methyl-phenyl)-4,4-dimethyl-pent-1-yn-3-ol

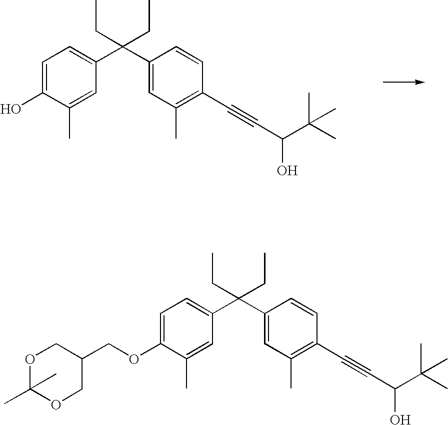

Using the same procedure as described for the preparation of Example 71-(1), the title compound was prepared from 4-{1-ethyl-1-[4-(3-hydroxy-4,4-dimethyl-pent-1-ynyl)-3-methyl-phenyl]-propyl}-2-methyl-phenol (compound prepared in Example 3). The yield was 68%.

¹H-NMR (300 MHz, CDCl₃): 0.59 (t, 6H), 1.06 (s, 9H), 1.43 (s, 3H), 1.46 (s, 3H), 1.76 (d, 1H), 2.10 (q, 4H), 2.13 (s, 3H), 2.16-2.23 (m, 1H), 2.38 (s, 3H), 3.92 (dd, 2H), 4.00 (d, 2H), 4.06 (dd, 2H), 4.25 (d, 2H), 6.68 (d, 1H), 6.84-7.00 (m, 4H), 7.27 (d, 1H); MS (ESI+): 524 ([M+NH₄]⁺).

### (2) Preparation of 2-(4-{1-ethyl-1-[4-(3-hydroxy-4,4-dimethyl-pent-1-ynyl)-3-methyl-phenyl]-propyl}-2-methyl-phenoxymethyl)-propane-1,3-diol

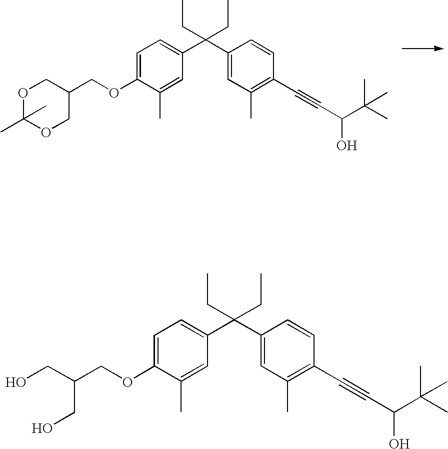

Using the same procedure as described for the preparation of Example 68-(2), the title compound was prepared from 1-(4-{1-[4-(2,2-dimethyl-[1,3]dioxan-5-ylmethoxy)-3-methyl-phenyl]-1-ethyl-propyl}-2-methyl-phenyl)-4,4-dimethyl-pent-1-yn-3-ol (compound prepared in Example 74-(1)). The yield was 57%.

1H-NMR (300 MHz, CDCl₃): 0.59 (t, 6H), 1.06 (s, 9H), 1.79 (d, 1H), 2.10 (q, 4H), 2.14 (s, 3H), 2.25 (m, 1H), 2.38 (s, 3H), 3.93-3.96 (m, 4H), 4.09 (d, 2H), 4.25 (d, 1H), 6.71 (d, 1H), 6.85-6.99 (m, 4H), 6.97-7.05 (m, 4H), 7.27 (d, 1H); MS (ESI−): 465 ([M−H]⁻).

### Example 75

### Preparation of 2-(4-{1-ethyl-1-[4-((E)-3-hydroxy-4,4-dimethyl-pent-1-enyl)-3-methyl-phenyl]-propyl}-2-methyl-phenoxymethyl)-propane-1,3-diol

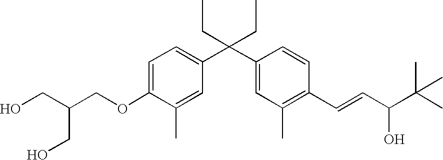

### (1) Preparation of (E)-1-(4-{1-[4-(2,2-dimethyl-[1,3]dioxan-5-ylmethoxy)-3-methyl-phenyl]-1-ethyl-propyl}-2-methyl-phenyl)-4,4-dimethyl-pent-1-en-3-ol

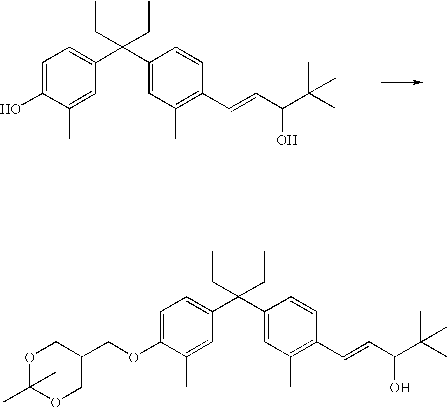

Using the same procedure as described for the preparation of Example 71-(1), the title compound was prepared from 4-{1-ethyl-1-[4-((E)-3-hydroxy-4,4-dimethyl-pent-1-enyl)-3-methyl-phenyl]-propyl}-2-methyl-phenol (compound prepared in Example 14). The yield was 53%.

1H-NMR (300 MHz, CDCl₃): 0.60 (t, 6H), 0.96 (s, 9H), 1.43 (s, 3H), 1.46 (s, 3H), 1.51 (d, 1H), 2.04 (q, 4H), 2.14 (s, 3H), 2.20 (m, 1H), 2.29 (s, 3H), 3.89-4.09 (m, 7H), 6.12 (dd, 1H), 6.68 (d, 1H), 6.73 (d, 1H), 6.90-6.96 (m, 4H), 7.31 (d, 1H); MS (ESI+) 526 ([M+NH₄]⁺).

### (2) Preparation of 2-(4-{1-ethyl-1-[4-((E)-3-hydroxy-4,4-dimethyl-pent-1-enyl)-3-methyl-phenyl]-propyl}-2-methyl-phenoxymethyl)-propane-1,3-diol

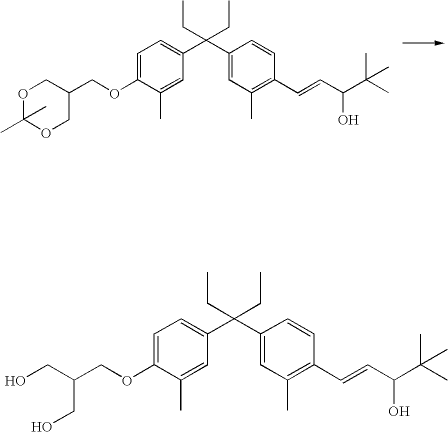

Using the same procedure as described for the preparation of Example 63-(2), the title compound was prepared from (E)-1-(4-{1-[4-(2,2-dimethyl-[1,3]dioxan-5-ylmethoxy)-3-methyl-phenyl]-1-ethyl-propyl}-2-methyl-phenyl)4,4-dimethyl-pent-1-en-3-ol (compound prepared in Example 75-(1)). The yield was 30%.

¹H-NMR (300 MHz, CDCl₃): 0.68 (t, 6H), 1.04 (s, 9H), 1.59 (m,1H), 2.13 (q, 4H), 2.22 (s, 3H), 2.29-2.37 (m, 1H), 2.37 (s, 3H), 3.984.04 (m, 5H), 4.17 (d, 2H), 6.19 (dd, 1H), 6.78-6.84 (m, 2H), 6.97-7.05 (m, 4H), 7.38 (d, 1H); MS (ESI−): 467 ([M−H]⁻).

### Example 76

### Preparation of 2-(4-{1-Ethyl-1-[4-(3-hydroxy-4,4-dimethyl-pentyl)-3-methyl-phenyl]-propyl}-2-methyl-phenoxymethyl)-propane-1,3-diol

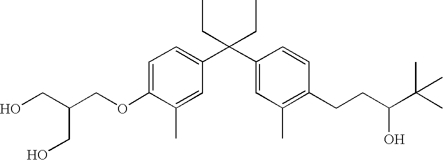

### Preparation of 2-(4-{1-ethyl-1-[4-(3-hydroxy-4,4-dimethyl-pentyl)-3-methyl-phenyl]-propyl}-2-methyl-phenoxymethyl)-propane-1,3-diol

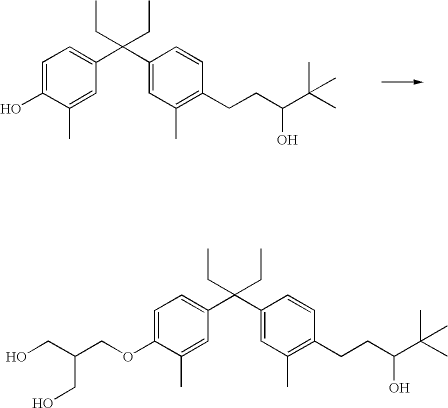

Using the same procedure as described for the preparation of Example 65, the title compound was prepared from 4-{1-Ethyl-1-[4-((E)-3-hydroxy-4,4-dimethyl-pent-1-enyl)-3-methyl-phenyl]-propyl}-2-methyl-phenol (compound prepared in Example 18) and toluene4-sulfonic acid 2,2-dimethyl-[1,3]dioxan-5-ylmethyl ester (prepared from following procedure as described in Tetrahedron, 1991, 47, 1001.). The yield was 36%.

¹H NMR (CDCl₃): 7.03-6.89 (m, 5H), 6.71 (d, 1H), 4.09 (d, 2H), 3.95 (d, 4H), 3.24 (dt, 1H), 2.85 (m, 1H), 2.55 (m, 1H), 2.25 (s, 3H), 2.15 (s, 3H), 2.04 (q, 4H), 1.79 (m,1H), 1.50 (m,1H), 0.89 (s, 9H), 0.59 (t, 6H); MS (ESI−) 469 ([M−H]⁻).

### Example 77

### Preparation of 2-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol

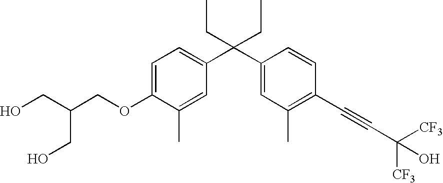

### (1) Preparation of 5-{4-[1-ethyl-1-(4-ethynyl-3-methyl-phenyl)-propyl]-2-methyl-phenoxymethyl}-2,2-dimethyl-[1,3]dioxane

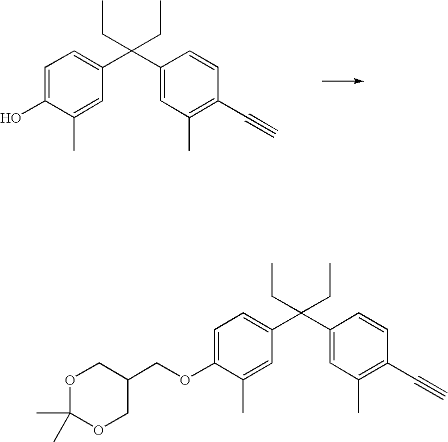

Using the same procedure as described for the preparation of Example 71-(1), the title compound was prepared from 4-[1-ethyl-1-(4-ethynyl-3-methyl-phenyl)-propyl]-2-methyl-phenol (compound prepared in Example 1-(3)). The yield was 73%.

¹H NMR (CDCl₃): 7.33 (d, 1H), 7.00-6.84 (m, 4H), 6.68 (d, 1H), 4.10-3.89 (m, 6H), 3.21 (s, 1H), 2.39 (s, 3H), 2.18-2.02 (m, 1H), 2.00 (s, 3H), 2.03 (q, 4H), 1.15 (s, 6H), 0.59 (t, 3H).

### (2) Preparation of 4-(4-1-[4-(2,2-Dimethyl-[1,3]dioxan-5-ylmethoxy)-3-methyl-phenyl]-1-ethyl-propyl}-2-methyl-phenyl)-1,1,1-trifluoro-2-trifluoromethyl-but-3-yn-2-ol

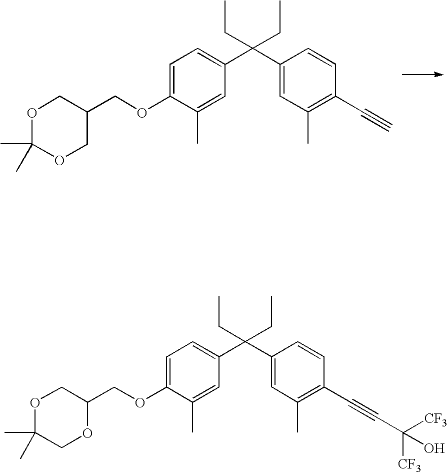

Using the same procedure as described for the preparation of Example 68-(2), the title compound was prepared from 5-{4-[1-Ethyl-1-(4-ethynyl-3-methyl-phenyl)-propyl]-2-methyl-phenoxymethyl}-2,2-dimethyl-[1,3]dioxane (compound prepared in Example 77-(1)). The yield was 69%.

¹H NMR (CDCl₃): 7.33 (d, 1H), 7.00-6.84 (m, 4H), 6.68 (d, 1H), 4.13-3.89 (m, 6H), 3.45 (s, 1H), 2.38 (s, 3H), 2.20-2.16 (m, 1H), 2.08 (s, 3H), 2.03 (q, 4H), 1.46 (s, 3H), 1.43 (s, 3H), 0.59 (t, 3H).

### (3) Preparation of 2-(4-{1-ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol

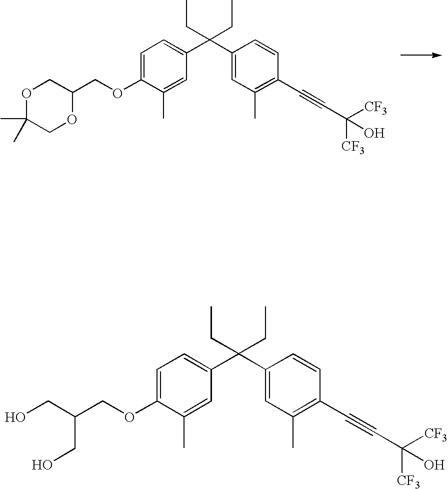

Using the same procedure as described for the preparation of Example 66-(2), the title compound was prepared from 4-(4-{1-[4-(2,2-dimethyl-[1,3]dioxan-5-ylmethoxy)-3-methyl-phenyl]-1-ethyl-propyl}-2-methyl-phenyl)-1,1,1-trifluoro-2-trifluoromethyl-but-3-yn-2-ol (compound prepared in Example 77-(2)). The yield was 69%.

¹H NMR (CDCl₃): 7.34 (d, 1H), 7.00-6.82 (m, 4H), 6.70 (d, 1H), 4.08 (d, 2H), 3.95 (m, 4H), 3.83 (s, 1H), 2.38 (s, 3H), 2.27-2.24 (m, 1H), 2.13 (s, 3H), 2.03 (q, 4H), 0.59 (t, 3H); MS (ESI−) 545 ([M−H]⁻).

### Example 78

### Preparation of 2-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol

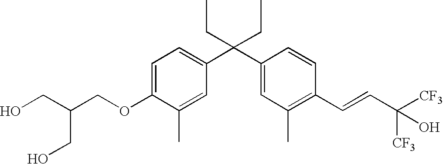

### (1) Preparation of (E)-4-(4-{1-[4-(2,2-dimethyl-[1,3]dioxan-5-ylmethoxy)-3-methyl-phenyl]-1-ethyl-propyl}-2-methyl-phenyl)-1,1,1-trifluoro-2-trifluoromethyl-but-3-en-2-ol

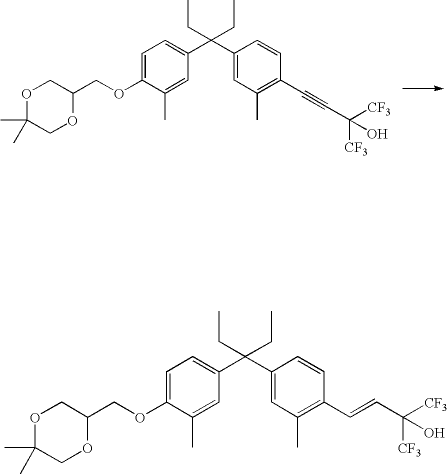

Using the same procedure as described for the preparation of Example 69-(1), the title compound was prepared from 4-(4-{1-[4-(2,2-dimethyl-[1,3]dioxan-5-ylmethoxy)-3-methyl-phenyl]-1-ethyl-propyl}-2-methyl-phenyl)-1,1,1-trifluoro-2-trifluoromethyl-but-3-yn-2-ol (compound prepared in Example 77-(2)). The yield was 39%.

¹H NMR (CDCl₃); 7.39-7.32 (m, 2H), 7.01-6.86 (m, 4H), 6.69 (d, 1H), 6.10 (d, 1H), 4.15-3.89 (m, 6H), 3.11 (s, 1H), 2.33 (s, 3H), 2.20-2.16 (m, 1H), 2.08 (s, 3H), 2.03 (q, 4H), 1.46 (s, 3H), 1.43 (s, 3H), 0.59 (t, 3H).

### (2) Preparation of 2-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol

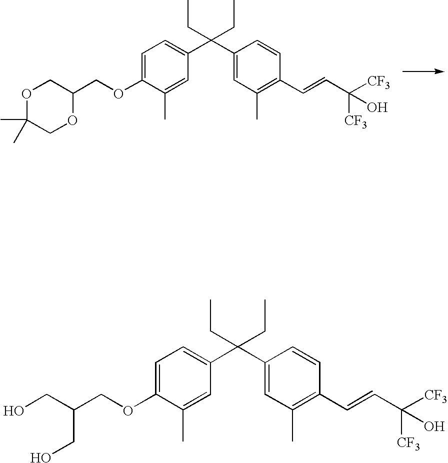

Using the same procedure as described for the preparation of Example 66-(2), the title compound was prepared from (E)-4-(4-{1-[4-(2,2-dimethyl-[1,3]dioxan-5-ylmethoxy)-3-methyl-phenyl]-1-ethyl-propyl}-2-methyl-phenyl)-1,1,1-trifluoro-2-trifluoromethyl-but-3-en-2-ol (compound prepared in Example 78-(1)). The yield was 83%.

¹H NMR (CDCl₃): 7.39-7.32 (m, 2H), 7.02-6.88 (m, 4H), 6.67 (d, 1H), 6.10 (d, 1H), 4.12 (d, 2H), 3.96 (d, 4H), 3.18 (s, 1H), 2.33 (s,.3H), 2.27-2.16 (m, 1H), 2.08 (s, 3H), 2.04 (q, 4H), 0.59 (t, 3H); MS (ESI−): 547 ([M−H]⁻).

### Example 79

### Preparation of 2-(4-{1-ethyl-1-[3-methyl-4-(4,4,4-trifluoro-3-hydroxy-3-trifluoromethyl-butyl)-phenyl]-propyl}-2-methyl-phenoxymethyl)-propane-1,3-diol

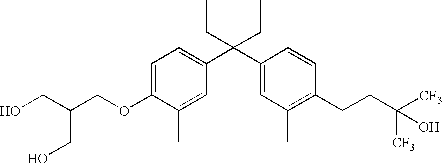

### Preparation of 2-(4-{1-ethyl-1-[3-methyl4-(4,4,4-trifluoro-3-hydroxy-3-trifluoromethyl-butyl)-phenyl]-propyl}-2-methyl-phenoxymethyl)-propane-1,3-diol

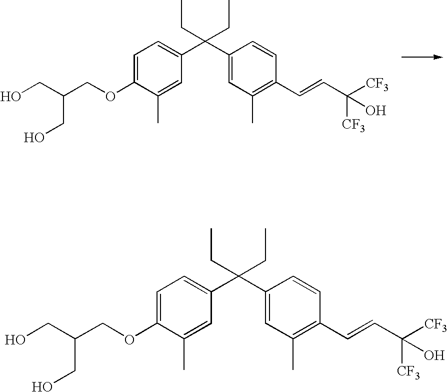

To a solution of 2-(4-{1-ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol (compound prepared in Example 78-(2)) (50 mg, 0.091 mmol) in MeOH (5 ml) was added Pd(OH)₂/C (5 mg, 10 wt %). The mixture was stirred at room temperature for 3 h under H₂ atmosphere. The mixture was filtered through celite 545, and the filtrate was concentrated under reduced pressure. The obtained residue was purified by silica gel chromatography (n-Hexane: CH₂Cl₂: EtOAc=1:1:1) to give the title compound (28.5 mg, 57%).

¹H NMR (CDCl₃): 7.01-6.90 (m, 5H), 6.72 (d, 1H), 6.67 (d, 1H), 6.10 (d, 1H), 4.09 (d, 2H), 3.96 (d, 2H), 3.95 (d, 2H), 3.13 (s, 1H), 2.79 (dd, 2H), 2.25 (s, 3H), 2.18-2.00 (m, 6H), 2.15 (s, 3H), 0.59 (t, 3H); MS (ESI−): 549 ([M−H]⁻).

### Example 80

### Preparation of 5-(4-{1-ethyl-1-[4-(3-hydroxy-4,4-dimethyl-pentyl)-3-methyl-phenyl]-propyl}-2-methyl-phenoxy)-pentanoic acid

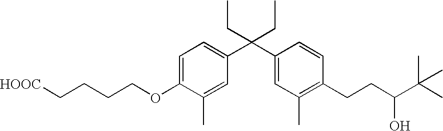

### (1) Preparation of 5-(4-{1-ethyl-1-[4-(3-hydroxy-4,4-dimethyl-pentyl)-3-methyl-phenyl]-propyl}-2-methyl-phenoxy)-pentanoic acid ethyl ester

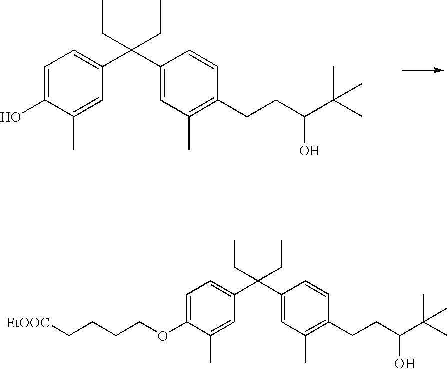

To a solution of 4-{1-ethyl-1-[4-(3-hydroxy-4,4-dimethyl-pentyl)-3-methyl-phenyl]-propyl}-2-methyl-phenol (compound prepared in Example 18) (200 mg, 0.52 mmol) in DMF (5 ml) were added K₂CO₃ (144 mg, 1.04 mmol) and ethyl-5-bromovalerate (0.169 ml, 1.04 mmol) and the mixture was stirred at 70 degrees C. for 13 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:10) to give the title compound (170 mg, 64%).

¹H-NMR (300 MHz, CDCl₃): 0.6 (t, 6H), 0.89 (s, 9H), 1.25 (t, 3H), 1.52 (m, 1H), 1.81 (m, 5H), 2.05 (q, 4H), 2.16 (s,3H), 2.26 (s,3H), 2.39 (m, 2H), 2.56 (m, 1H), 2.85 (m, 1H), 3.26 (m, 1H), 3.94 (t, 2H), 4.12 (q, 2H), 6.66 (d, 1H), 6.90 (m. 4H), 7.02 (d, 1H).

### (2) Preparation of 5-(4-{1-ethyl-1-[4-(3-hydroxy-4,4-dimethyl-pentyl)-3-methyl-phenyl]-propyl}-2-methyl-phenoxy)-pentanoic acid

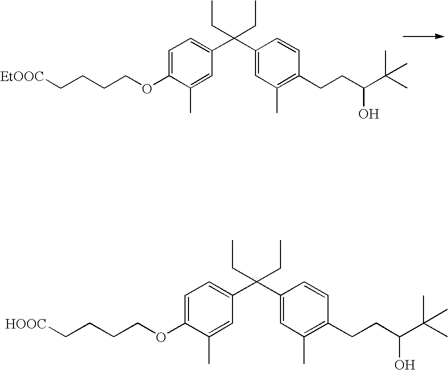

To a stirred solution of 5-(4-{1-ethyl-1-[4-(3-hydroxy-4,4-dimethyl-pentyl)-3-methyl-phenyl]-propyl}-2-methyl-phenoxy)-pentanoic acid ethyl ester (compound prepared in Example 80-(1)) (170 mg, 0.333 mmol) in MeOH (3 ml) was added 1N—KOH (1.0 ml, 1.0 mmol) and the mixture was stirred at room temperature for 5 h. The reaction mixture was poured into 1N—HCl and the products were extracted with EtOAc. The extracts were washed with brine, dried over MgSO₄, filtered and concentrated under reduced pressure. The obtained residue was chromatographed on silica gel with EtOAc-n-Hexane (1:3) to give the title compound (108 mg, 67%).

¹H-NMR (300 MHz, CDCl₃): 0.6 (t, 6H), 0.89 (s, 9H), 1.52 (m, 1H), 1.81 (m, 1H), 1.86 (m, 4H), 2.05 (q, 4H), 2.16 (s,3H), 2.26 (s,3H), 2.46 (m, 2H), 2.56 (m, 1H), 2.85 (m, 1H), 3.26 (m,1H), 3.94 (t, 2H), 6.66 (d, 1H), 6.90 (m, 4H), 7.02 (d, 1H); MS (ESI−): 481 ([M−H]⁻).

### Example 81

### Preparation of 5-(4-{1-Ethyl-1-[4-((E)-3-ethyl-3-hydroxy-pent-1-enyl)-3-methyl-phenyl]-propyl}-2-methyl-phenoxy)-pentanoic acid

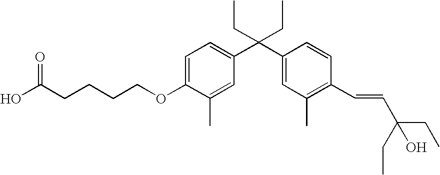

### (1) Preparation of 5-(4-{1-ethyl-1-[4-((E)-3-ethyl-3-hydroxy-pent-1-enyl)-3-methyl-phenyl]-propyl}-2-methyl-phenoxy)-pentanoic acid ethyl ester

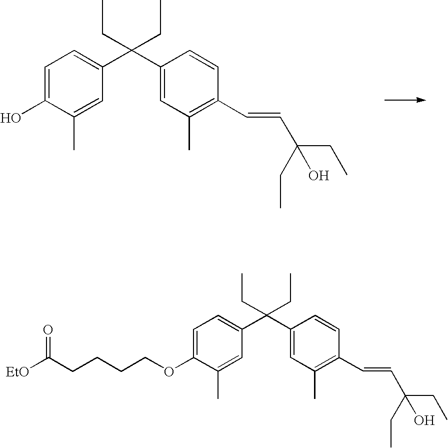

Using the same procedure as described for the preparation of Example 80-(1), the title compound was prepared from 4-{1-ethyl-1-[4-((E)-3-ethyl-3-hydroxy-pent-1-enyl)-3-methyl-phenyl]-propyl}-2-methyl-phenol (compound prepared in Example 1-(5)). The yield was 87%.

¹H-NMR (300 MHz, CDCl₃): 0.61 (t, 6H), 0.92 (t, 6H), 1.25 (t, 3H), 1.64 (q, 4H), 1.84 (m, 4H), 2.04 (q, 4H), 2.16 (s, 3H), 2.31 (s, 3H), 2.39 (t, 2H), 3.95 (t, 2H), 4.14 (q, 2H), 5.99 (d, 1H), 6.66 (d, 1H), 6.74 (d, 1H), 6.91-6.96 (m, 4H), 7.29 (d, 1H); MS (ESI+): 531 ([M+Na]⁺).

### (2) Preparation of 5-(4-{1-ethyl-1-[4-((E)-3-ethyl-3-hydroxy-pent-1-enyl)-3-methyl-phenyl]-propyl}-2-methyl-phenoxy)-pentanoic acid

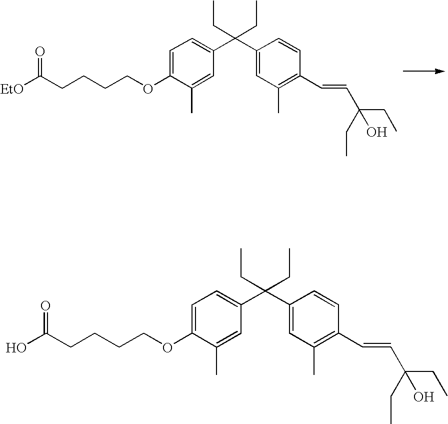

Using the same procedure as described for the preparation of Example 80-(2), the title compound was prepared from 5-(4-{1-ethyl-1-[4-((E)-3-ethyl-3-hydroxy-pent-1-enyl)-3-methyl-phenyl]-propyl}-2-methyl-phenoxy)-pentanoic acid ethyl ester (compound prepared in Example 81-(1)). The yield was 82%.

¹H-NMR (300 MHz, CDCl₃): 0.61 (t, 6H), 0.92 (t, 6H), 1.64 (q, 4H), 1.85 (m, 1H), 2.04 (q, 4H), 2.15 (s, 3H), 2.31 (s, 3H), 2.46 (t, 2H), 3.95 (t, 2H), 5.99 (d, 1H), 6.66 (d, 1H), 6.74 (d,1H), 6.91-6.96 (m, 4H), 7.29 (d,1H) ; MS (ESI−) 479 ([M−H]⁻).

### Example 82

### Preparation of 5-(4-{l-ethyl-i-[4-(3-ethyl-3-hydroxy-pent-1-ynyl)-3-methyl-phenyl]-propyl}-2-methyl-phenoxy)-pentanoic acid

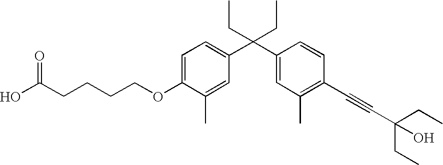

### Preparation of 5-(4-{1-ethyl-1-[4-(3-ethyl-3-hydroxy-pent-1-ynyl)-3-methyl-phenyl]-propyl}-2-methyl-phenoxy)-pentanoic acid

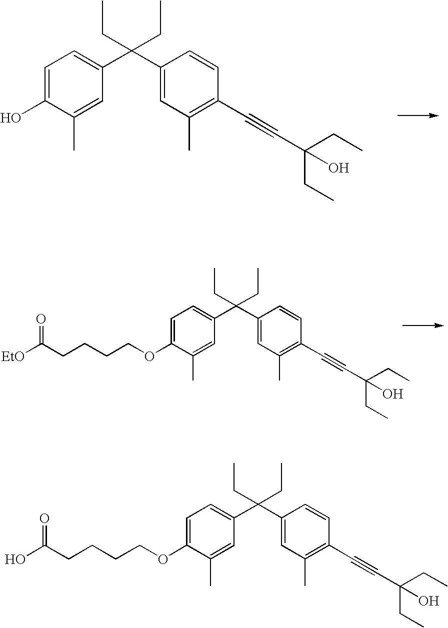

Using the same procedure as described for the preparation of Example 80-(1), 5-(4-{1-ethyl-1-[4-(3-ethyl-3-hydroxy-pent-1-ynyl)-3-methyl-phenyl]-propyl}-methyl-phenoxy)-pentanoic acid ethyl ester was prepared from 4-{1-ethyl-1-[4-(3-ethyl-3-hydroxy-pent-1-ynyl)-3-methyl-phenyl]-propyl}-2-methyl-phenol (compound prepared in Example 1-(4)). The yield was 53%.

Using the same procedure as described for the preparation of Example 80-(2), the title compound was prepared from 5-(4-{1-ethyl-1-[4-(3-ethyl-3-hydroxy-pent-1-ynyl)-3-methyl-phenyl]-propyl}-2-methyl-phenoxy)-pentanoic acid ethyl ester. The yield was 88%.

¹H-NMR (300 MHz, CDCl₃): 0.59 (t, 6H), 1.11 (t, 6H), 1.75-1.87 (m, 8H), 2.05 (q, 4H), 2.37 (s, 3H), 2.39 (s, 3H), 2.46 (td, 2H), 3.95 (t, 2H), 6.65 (d, 1H), 6.84 (s, 1H), 6.91 (td, 2H), 7.00 (s, 1H), 7.27 (d, 1H); MS (ESI−): 477 ([M−H]⁻).

### Example 83

### Preparation of 5-(4-{1-ethyl-1-[3-methyl-4-(4,4,4-trifluoro-3-hydroxy-3-trifluoromethyl-but-1-ynyl)-phenyl]-propyl}-2-methyl-phenoxy)-pentanoic acid

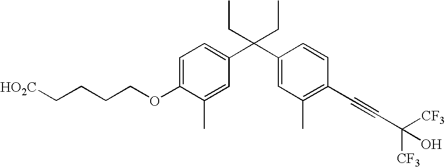

### (1) Preparation of 5-(4-{1-Ethyl-1-[3-methyl4-(4,4,4-trifluoro-3-methoxymethoxy-3-trifluoromethyl-but-1-ynyl)-phenyl]-propyl}-2-methyl-phenoxy)-pentanoic acid ethyl ester

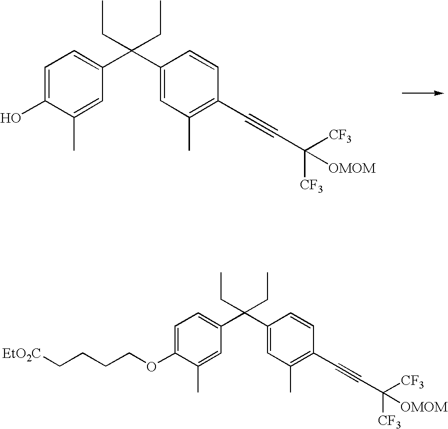

Using the same procedure as described for the preparation of Example 80-(1), the title compound was prepared from 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)). The yield was 67%.

¹H-NMR (300 MHz, CDCl₃): 7.40 (d, 1H), 7.08 (bs, 1H), 7.00 (dd, 1H), 6.90 (dd, 1H), 6.85 (bd, 1H), 6.70 (d, 1H), 5.15.(s, 2H), 4.15 (q, 2H), 3.95 (m, 2H), 3.48 (s, 3H), 2.45-2.30 (m, 5H), 2.15 (s, 3H), 2.05 (q, 4H), 1.90-1.75 (m, 4H), 1.25 (t, 3H), 0.60 (t, 6H).

### (2) Preparation of 5-(4-{1-ethyl-1-[3-methyl4-(4,4,4-trifluoro-3-hydroxy-3-trifluoromethyl-but-1-ynyl)-phenyl]-propyl}-2-methyl-phenoxy)-pentanoic acid ethyl ester

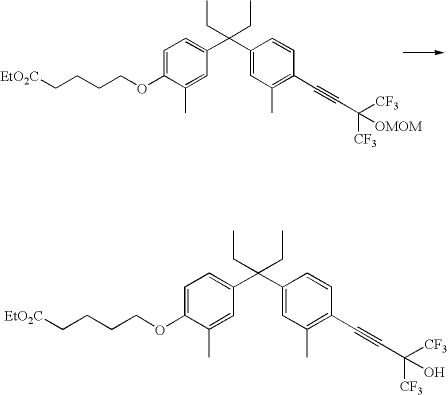

To a stirred solution of 5-(4-{1-ethyl-1-[3-methyl4-(4,4,4-trifluoro-3-methoxymethoxy-3-trifluoromethyl-but-1-ynyl)-phenyl]-propyl}-2-methyl-phenoxy)-pentanoic acid ethyl ester (compound prepared in Example 83-(1)) (147 mg, 0.23 mmol) in i-PrOH (1.2 ml) was added CBr₄(7.60 mg, 10 mol %) under N₂ atmosphere at room temperature and the reaction mixture was refluxed for 6.0 h. The reaction mixture was poured into water and the products were extracted with EtOAc. The extracts were washed with water and brine, dried over anhydrous Na₂SO₄, filtered and evaporated under reduced pressure. The obtained residue was purified by silica gel chromatography (n-Hexane:EtOAc=6:1) to give the title compound (140 mg, 86%) as colorless sticky oil.

¹H-NMR (300 MHz, CDCl₃): 7.40 (d, 1H), 7.08 (bs, 1H), 7.00 (dd, 1H), 6.90 (dd, 1H), 6.85 (bd, 1H), 6.70 (d, 1H), 4.15 (q, 2H), 3.95 (m, 3H), 2.45-2.30 (m, 5H), 2.15 (s, 3H), 2.05 (q, 4H), 1.90-1.75 (m, 4H), 1.25 (t, 3H), 0.60 (t, 6H); MS (ESI−): 585 ([M−H]⁻).

### (3) Preparation of 5-(4-{1-ethyl-1-[3-methyl4-(4,4,4-trifluoro-3-hydroxy-3-trifluoromethyl-but-1-ynyl)-phenyl]-propyl}-2-methyl-phenoxy)-pentanoic acid

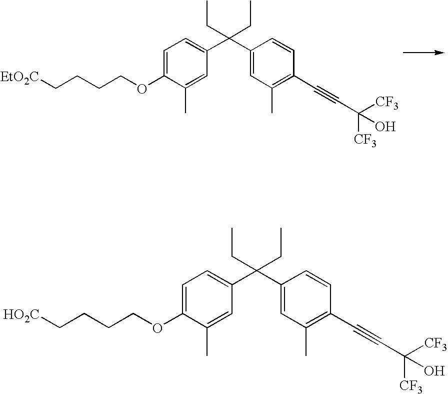

Using the same procedure as described for the preparation of Example 80-(2), the title compound was prepared from 5-(4-{1-ethyl-1-[3-methyl4-(4,4,4-trifluoro-3-hydroxy-3-trifluoromethyl-but-1-ynyl)-phenyl]-propyl}-2-methyl-phenoxy)-pentanoic acid ethyl ester (compound prepared in Example 83-(2)). The yield was 80%.

¹H-NMR(300 MHz, CDCl₃): 7.40 (d, 1H), 7.08 (bs, 1H), 7.00 (dd, 1H), 6.90 (dd, 1H), 6.85 (bd, 1H), 6.70 (d, 1H), 3.95 (m, 2H), 3.80 (m, 1H), 2.50-2.35 (m, 5H), 2.15 (s, 3H), 2.05 (q, 4H), 1.90-1.75 (m, 4H), 0.60 (t, 6H); MS (ESI−): 557 ([M−H]⁻).

### Example 84

### Preparation of 5-(4-{1-Ethyl-1-[3-methyl4-((E)-4,4,4-trifluoro-3-hydroxy-3-trifluoromethyl-but-1-enyl)-phenyl]-propyl}-2-methyl-phenoxy)-pentanoic acid

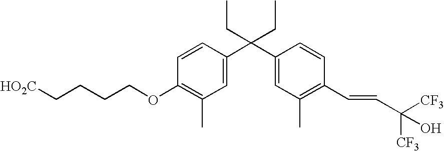

### (1) Preparation of 5-(4-{1-ethyl-1-[3-methyl4-((E)-4,4,4-trifluoro-3-methoxymethoxy-3-trifluoromethyl-but-1-enyl)-phenyl]-propyl}-2-methyl-phenoxy)-pentanoic acid ethyl ester

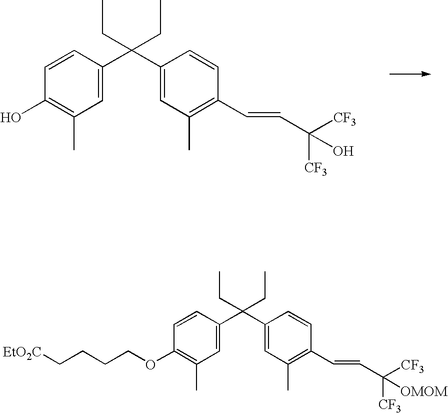

Using the same procedure as described for the preparation of Example 80-(1), the title compound was prepared from 4-{1-ethyl-1-[3-methyl4-((E)-4,4,4-trifluoro-3-methoxymethoxy-3-trifluoromethyl-but-1-enyl)-phenyl]-propyl}-2-methyl-phenol (compound prepared in Example 30-(1)). The yield was 84%.

¹H-NMR (300 MHz, CDCl₃): 0.61 (t, 6H), 1.25 (t, 3H), 1.83 (m, 4H), 2.05 (q, 4H), 2.16 (s, 3H), 2.31 (s, 3H), 2.39 (m, 2H), 3.50 (s, 3H), 3.94 (m, 2H), 4.12 (q, 2H), 4.96 (s, 2H), 6.06 (d, 1H), 6.67 (d, 1H), 6.88-7.03 (m, 4H), 7.35 (m, 2H).

### (2) Preparation of 5-(4-{1-ethyl-1-[3-methyl4-((E)-4,4,4-trifluoro-3-hydroxy-3-trifluoromethyl-but-1-enyl)-phenyl]-propyl}-2-methyl-phenoxy)-pentanoic acid ethyl ester

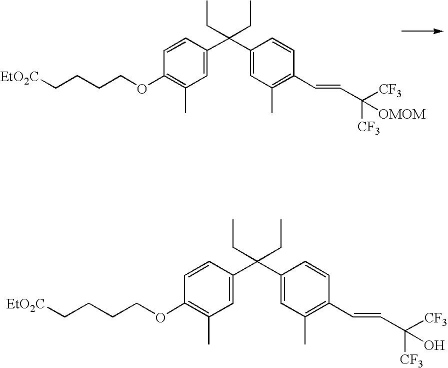

Using the same procedure as described for the preparation of Example 83-(2), the title compound was prepared from 5-(4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-pentanoic acid ethyl ester (compound prepared in Example 84-(I)). The yield was 84%.

¹H-NMR (300 MHz, CDCl₃): 0.61 (t, 6H), 1.25 (t, 3H), 1.83 (m, 4H), 2.05 (q, 4H), 2.15 (s, 3H), 2.34 (s, 3H), 2.39 (m, 2H), 3.18 (s, 1H), 3.94 (m, 2H), 4.12 (q, 2H), 6.08 (d, 1H), 6.67 (d, 1H), 6.87-7.03 (m, 4H), 7.35 (m, 2H).

### (3) Preparation of 5-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid

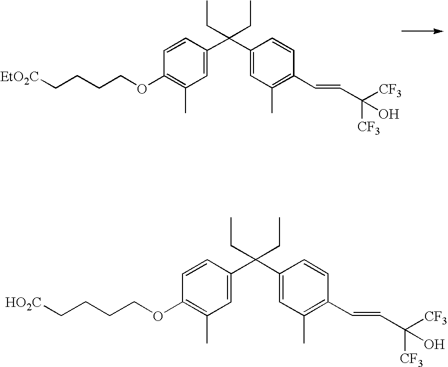

Using the same procedure as described for the preparation of Example 80-(2), the title compound was prepared from 5-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid ethyl ester (compound prepared in Example 84-(2)). The yield was 81%.

¹H-NMR (300 MHz, CDCl₃): 0.61 (t, 6H), 1.26 (t, 3H), 1.85 (m, 4H), 2.05 (q, 4H), 2.15 (s, 3H), 2.33 (s, 3H), 2.46 (m, 2H), 3.95 (m, 2H), 6.08 (d, 1H), 6.67 (d, 1H), 6.88-7.02 (m, 3H), 7.26-7.39 (m, 3H); MS (ESI−): 559 ([M−H]⁻).

### Example 85

### Preparation of 5-(4-{1-Ethyl-1-[4-(I-hydroxy-cyclopentylethynyl)-3-methyl-phenyl]-propyl}-2-methyl-phenoxy)-pentanoic acid

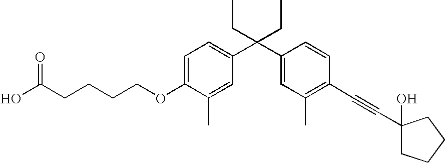

### (1) Preparation of 5-(4-{1-ethyl-1-[4-(1-hydroxy-cyclopentylethynyl)-3-methyl-phenyl]-propyl}-2-methyl-phenoxy)-pentanoic acid ethyl ester

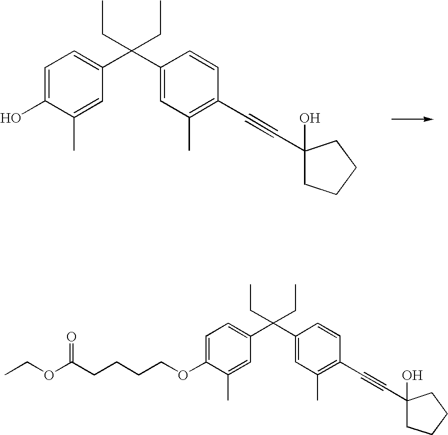

Using the same procedure as described for the preparation of Example 80-(1), the title compound was prepared from 4-{1-ethyl-1-[4-(1-hydroxy-cyclopentylethynyl)-3-methyl-phenyl]-propyl}-2-methyl-phenol (compound prepared in Example 7). The yield was 84%.

¹H-NMR (CDCl₃): 7.27 (d, 1H), 7.0 (s, 1H), 6.92 (m, 2H), 6.84 (dd,1H), 6.65 (d, 1H), 4.12 (q, 2H), 3.94 (m, 2H), 2.39 (m, 2H), 2.36 (s, 3H), 2.14 (s, 3H), 2.03 (m, 6H), 1.84 (m, 8H), 1.25 (m, 6H), 0.59 (t, 6H).

### (2) Preparation of 5-(4-{1-ethyl-1-[4-(1-hydroxy-cyclopentylethynyl)-3-methyl-phenyl]-propyl}-2-methyl-phenoxy)-pentanoic acid

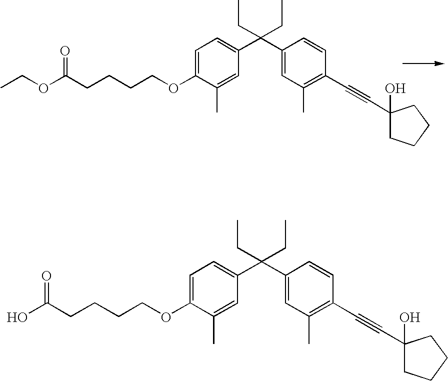

Using the same procedure as described for the preparation of Example 80-(2), the title compound-was prepared from 5-(4-{1-ethyl-1-[4-(1-hydroxy-cyclopentylethynyl)-3-methyl-phenyl]-propyl}-2-methyl-phenoxy)-pentanoic acid ethyl ester (compound prepared in Example 85-(1)). The yield was 31%.

¹H-NMR (CDCl₃): 7.26 (d, 1H), 7.0 (s, 1H), 6.92 (m, 2H), 6.84 (dd, 1H), 6.65 (d, 1H), 3.94 (m, 2H), 2.45 (m, 2H), 2.36 (s, 3H), 2.14 (s, 3H), 2.03 (m, 8H), 1.85 (m, 8H), 0.58 (t, 6H); MS (ESI−): 475 ([M−H]⁻).

### Example 86

### Preparation of 5-[4-(1-ethyl-1-{4-[(E)-2-(1-hydroxy-cyclopentyl)-vinyl]-3-methyl-phenyl}-propyl)-2-methyl-phenoxy]-pentanoic acid

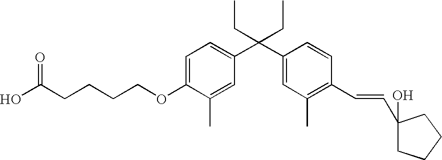

### (1) Preparation of 5-[4-(1-ethyl-1-{4-[(E)-2-(1-hydroxy-cyclopentyl)-vinyl]-3-methyl-phenyl}-propyl)-2-methyl-phenoxy]-pentanoic acid ethyl ester

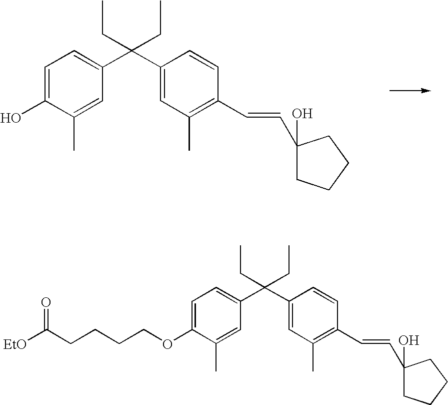

Using the same procedure as described for the preparation of Example 80-(1), the title compound was prepared from 4-(1-ethyl-1-{4-[(E)-2-(1-hydroxy-cyclopentyl)-vinyl]-3-methyl-phenyl}-propyl)-2-methyl-phenol (compound prepared in Example 10). The yield was 51%.

¹H-NMR (300 MHz, CDCl₃): 0.58 (t, 6H), 1.25 (t, 3H), 1.48-1.91 (m, 12H), 2.04 (q, 4H), 2.13 (s, 3H), 2.36 (s, 3H), 2.32 (t, 2H), 3.92 (t, 2H), 4.12 (q, 2H), 6.21 (d, 1H), 6.69 (d, 1H), 6.84 (d, 1H), 6.87-7.00 (m, 4H), 7.35 (d, 1H).

### (2) Preparation of 5-[4-(1-ethyl-1-{4-[(E)-2-(1-hydroxy-cyclopentyl)-vinyl]-3-methyl-phenyl}-propyl)-2-methyl-phenoxy]-pentanoic acid

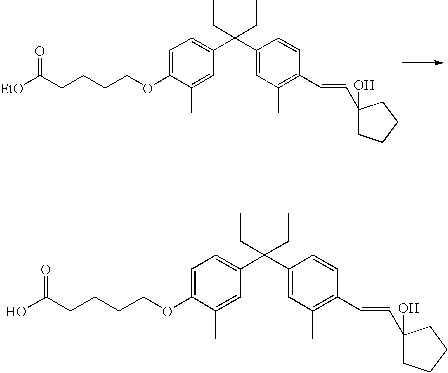

Using the same procedure as described for the preparation of Example 80-(2), the title compound was prepared from 5-[4-(1-ethyl-1-{4-[(E)-2-(1-hydroxy-cyclopentyl)-vinyl]-3-methyl-phenyl}-propyl)-2-methyl-phenoxy]-pentanoic acid ethyl ester (compound prepared in Example 86-(1)). The yield was 79%.

¹H-NMR (300 MHz, CDCl₃): 0.60 (t, 6H), 1.32-1.79 (m, 12H), 2.05 (q, 4H), 2.15 (s, 3H), 2.31 (s, 3H), 2.46 (t, 2H), 3.95 (t, 2H), 6.25 (d, 1H), 6.65 (d, 1H), 6.81-6.95 (m, 5H), 7.32 (d, 1H); MS (ESI−): 477 ([M−H]⁻).

### Example 87

### Preparation of 5-(4-{1-ethyl-i-[4-(1-hydroxy-cyclohexylethynyl)-3-methyl-phenyl]-propyl}-2-methyl-phenoxy)-pentanoic acid

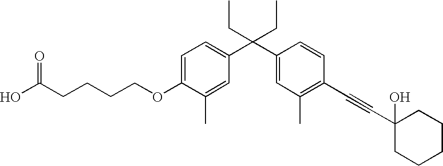

### (1) Preparation of 5-(4-{1-ethyl-1-[4-(1-hydroxy-cyclohexylethynyl)-3-methyl-phenyl]-propyl}-2-methyl-phenoxy)-pentanoic acid ethyl ester

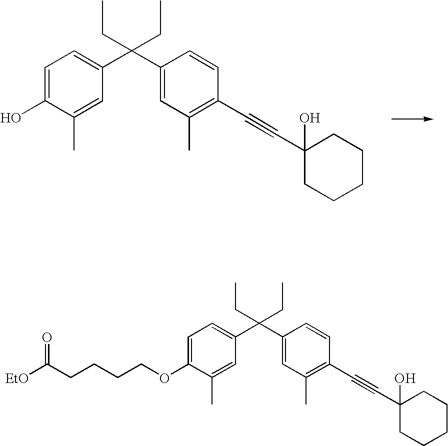

Using the same procedure as described for the preparation of Example 80-(1), the title compound was prepared from 4-{1-ethyl-1-[4-(1-hydroxy-cyclohexylethynyl)-3-methyl-phenyl]-propyl}-2-methyl-phenol (compound prepared in Example 8). The yield was 51%.

¹H-NMR (300 MHz, CDCl₃): 0.59 (t, 6H), 1.25 (t, 3H), 1.52-1.79 (m, 8H), 1.85 (q, 4H), 2.03 (m, 6H), 2.15 (s, 3H), 2.38 (s, 3H), 2.38 (t, 2H), 3.95 (t, 2H), 6.66 (d, 1H), 6.84 (s, 1H), 6.92 (td, 2H), 7.00 (s, 1H), 7.27 (d, 1H).

### (2) Preparation of 5-(4-{1-ethyl-1-[4-(1-hydroxy-cyclohexylethynyl)-3-methyl-phenyl]-propyl}-2-methyl-phenoxy)-pentanoic acid

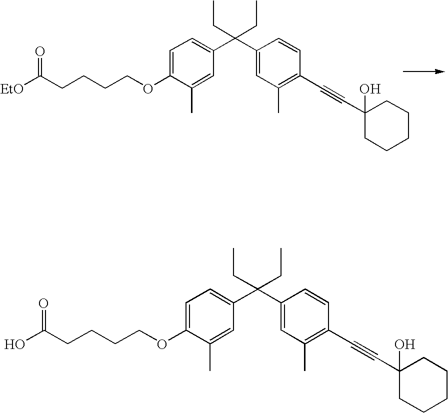

Using the same procedure as described for the preparation of Example 80-(2), the title compound was prepared from 5-(4-{1-Ethyl-1-[4-(1-hydroxy-cyclohexylethynyl)-3-methyl-phenyl]-propyl}-2-methyl-phenoxy)-pentanoic acid ethyl ester (compound prepared in Example 87-(1)). The yield was 80%.

¹H-NMR (300 MHz, CDCl₃): 0.59 (t, 6H), 1.25 (m, 1H), 1.52-1.79 (m, 7H), 1.85 (m, 4H), 2.03 (q, 6H), 2.14 (s, 3H), 2.38 (s, 3H), 2.46 (t, 2H), 3.95 (t, 2H), 6.66 (d, 1H), 6.84 (s, 1H), 6.92 (td, 2H), 7.00 (s, 1H), 7.27 (d, 1H); MS (ESI−): 489 ([M−H]⁻).

### Example 88

### Preparation of 5-[4-(1-ethyl-1-{4-[(E)-2-(1-hydroxy-cyclohexyl)-vinyl]-3-methyl-phenyl}-propyl)-2-methyl-phenoxy]-pentanoic acid

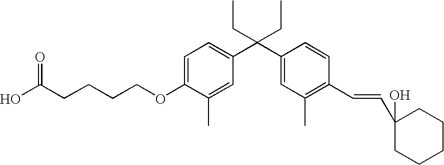

### (1) Preparation of 5-[4-(1-ethyl-1-{4-[(E)-2-(1-hydroxy-cyclohexyl)-vinyl]-3-methyl-phenyl}-propyl)-2-methyl-phenoxy]-pentanoic acid ethyl ester

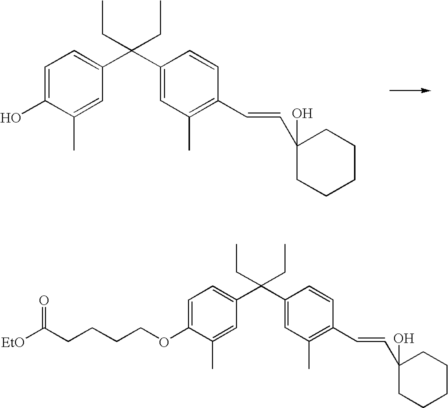

Using the same procedure as described for the preparation of Example 80-(1), the title compound was prepared from 4-(1-Ethyl-1-{4-[(E)-2-(1-hydroxy-cyclohexyl)-vinyl]-3-methyl-phenyl}-propyl)-2-methyl-phenol (compound prepared in Example 11). The yield was 64%.

¹H-NMR (300 MHz, CDCl₃): 0.59 (t, 6H), 1.25 (t, 3H), 1.31-1.79 (m, 10H), 1.85 (m, 4H), 2.03 (q, 4H), 2.14 (s, 3H), 2.38 (s, 3H), 2.38 (t, 2H), 3.99 (t, 2H), 4.18 (q, 2H), 6.21 (d, 1H), 6.69 (d, 1H), 6.84 (d, 1H), 6.87-7.00 (m, 4H), 7.35 (d, 1H).

### (2) Preparation of 5-[4-(1-ethyl-1-(4-[(E)-2-(1-hydroxy-cyclohexyl)-vinyl]-3-methyl-phenyl}-propyl)-2-methyl-phenoxy]-pentanoic acid

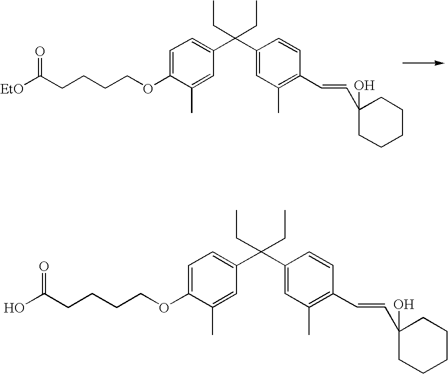

Using the same procedure as described for the preparation of Example 80-(2), the title compound was prepared from 5-[4-(1-ethyl-1-{4-[(E)-2-(1-hydroxy-cyclohexyl)-vinyl]-3-methyl-phenyl}-propyl)-2-methyl-phenoxy]-pentanoic acid ethyl ester (compound prepared in Example 88-(1)). The yield was 83%.

¹H-NMR (300 MHz, CDCl₃): 0.59 (t, 6H), 1.32 (m, 1H), 152-1.79 (m, 9H), 1.85 (m, 4H), 2.03 (q, 4H), 2.14 (s, 3H), 2.38 (s, 3H), 2.46 (t, 2H), 3.95 (t, 2H), 6.20 (d, 1H), 6.69 (d, 1H), 6.81 (d, 1H), 6.87-7.00 (m, 4H), 7.31 (d, 1H); MS (ESI−): 491 ([M−H]⁻).

### Example 89

### Preparation of 6-(4-{1-ethyl-1-[4-(3-ethyl-3-hydroxy-pent-1-ynyl)-3-methyl-phenyl]-propyl}-2-methyl-phenoxy)-hexanoic acid

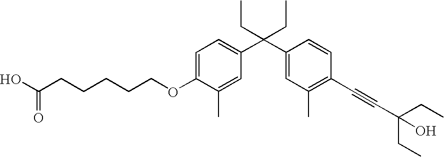

### (1) Preparation of 6-(4-{1-ethyl-1-[4-(3-ethyl-3-hydroxy-pent-1-ynyl)-3-methyl-phenyl]-propyl}-2-methyl-phenoxy)-hexanoic acid ethyl ester

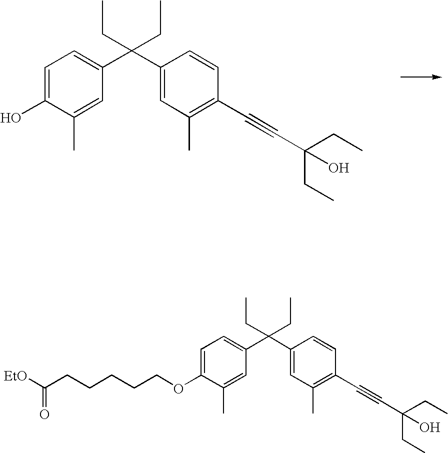

Using the same procedure as described for the preparation of Example 80-(1), the title compound was prepared from 4-{1-ethyl-1-[4-(3-ethyl-3-hydroxy-pent-1-ynyl)-3-methyl-phenyl]-propyl}-2-methyl-phenol (compound prepared in Example 1-(4)) and ethyl-6-bromohexanoate. The yield was 64%.

¹H-NMR (300 MHz, CDCl₃): 0.59 (t, 6H), 1.11 (t, 6H), 1.25 (t, 3H), 1.44-1.57 (m, 2H), 1.64-1.85 (m, 8H), 1.98-2.07 (m, 4H), 2.14 (s, 3H), 2.33 (t, 2H), 2.37 (s, 3H), 3.92 (t, 2H), 4.13 (q, 2H), 6.60 (d, 1H, J=8.4 Hz), 6.83-6.95 (m, 3H), 7.00 (s, 1H), 7.27 (d, 1H, J=8.0 Hz).

### (2) Preparation of 6-(4-{1-ethyl-1-[4-(3-ethyl-3-hydroxy-pent-1-ynyl)-3-methyl-phenyl]-propyl}-2-methyl-phenoxy)-hexanoic acid

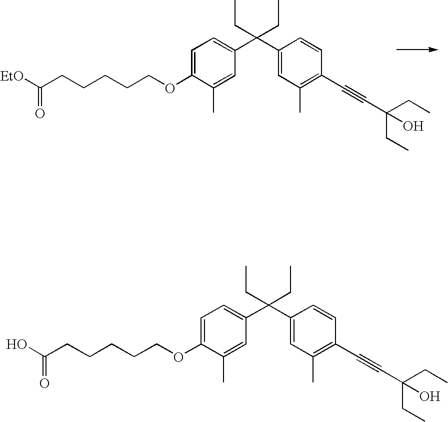

Using the same procedure as described for the preparation of Example 80-(2), the title compound was prepared from 6-(4-{1-ethyl-1-[4-(3-ethyl-3-hydroxy-pent-1-ynyl)-3-methyl-phenyl]-propyl}-2-methyl-phenoxy)-hexanoic acid ethyl ester (compound prepared in Example 89-(1)). The yield was 85%.

¹H-NMR (300 MHz, CDCl₃): 0.59 (t, 6H), 1.11 (t, 6H), 1.49-1.59 (m, 2H), 1.67-1.85 (m, 8H), 2.03 (q, 4H), 2.14 (s, 3H), 2.36-2.42 (m, 5H), 3.92 (t, 2H), 6.66 (d, 1H, J=8.4 Hz), 6.84-6.95 (m, 3H), 7.90 (s, 1H), 7.26 (d, 1H, J=8.0 Hz); MS (ESI−): 491 ([M−H]⁻).

### Example 90

### Preparation of 6-(4-{1-ethyl-1-[4-((E)-3-ethyl-3-hydroxy-pent-1-enyl)-3-methyl-phenyl]-propyl}-2-methyl-phenoxy)-hexanoic acid

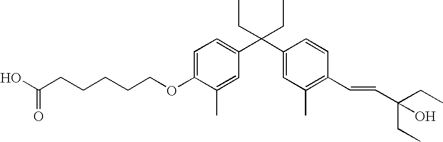

### (1) Preparation of 6-(4-{1-ethyl-1-[4-((E)-3-ethyl-3-hydroxy-pent-1-enyl)-3-methyl-phenyl]-propyl}-2-methyl-phenoxy)-hexanoic acid ethyl ester

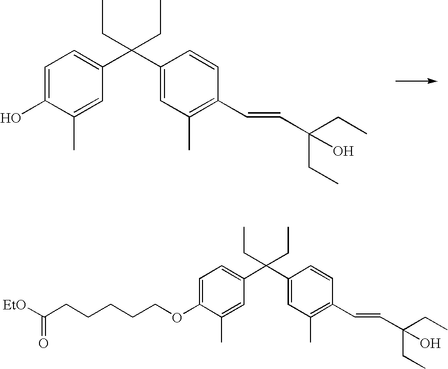

Using the same procedure as described for the preparation of Example 80-(1), the title compound was prepared from 4-{1-ethyl-1-[4-((E)-3-ethyl-3-hydroxy-pent-1-enyl)-3-methyl-phenyl]-propyl}-2-methyl-phenol (compound prepared in Example 1-(5)) and ethyl-6-bromohexanoate. The yield was 75%.

¹H-NMR (300 MHz, CDCl₃): 0.61 (t, 6H), 0.91 (t, 6H),1.25 (t, 3H), 1.47-1.85 (m, 10H), 2.05 (q, 4H), 2.15 (s,3H), 2.30 (s,3H), 2.39 (t, 2H), 3.95 (t 2H), 4.13 (q, 2H), 6.00 (d, 1H, J=16.0 Hz), 6.66 (d, 1H, J=8.4 Hz), 6.74 (d, 1H, J=16.0 Hz), 6.90-6.96 (m, 4H), 7.29 (d, 1H, J=8.7 Hz).

### (2) Preparation of 6-(4-{1-ethyl-1-[4-((E)-3-ethyl-3-hydroxy-pent-1-enyl)-3-methyl-phenyl]-propyl}-2-methyl-phenoxy)-hexanoic acid

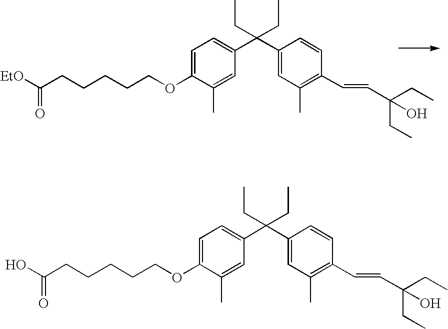

Using the same procedure as described for the preparation of Example 80-(2), the title compound was prepared from 6-(4-{1-Ethyl-1-[4-((E)-3-ethyl-3-hydroxy-pent-1-enyl)-3-methyl-phenyl]-propyl}-2-methyl-phenoxy)-hexanoic acid ethyl ester (compound prepared in Example 90-(1)). The yield was 75%.

¹H-NMR (300 MHz, CDCl₃): 0.61 (t, 6H), 0.91 (t, 6H), 1.49-1.85 (m, 10H), 2.04 (q, 4H), 2.15 (s,3H), 2.30 (s,3H), 2.39 (t, 2H), 3.93 (t 2H), 6.00 (d, 1H, J=16.0 Hz), 6.66 (d, 1H, J=8.4 Hz), 6.74 (d, 1H, J=16.0 Hz), 6.90-6.96 (m, 4H), 7.29 (d, 1H, J=8.7 Hz); MS (ESI−): 493 ([M−H]⁻).

### Example 91

### Preparation of 6-(4-{1-ethyl-1-[3-methyl4-(4,4,4-trifluoro-3-hydroxy-3-trifluoromethyl-but-1-ynyl)-phenyl]-propyl}-2-methyl-phenoxy)-hexanoic acid

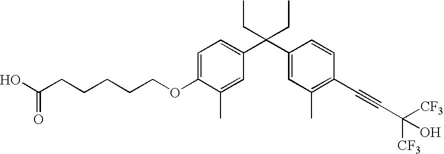

### (1) Preparation of 6-(4-{1-ethyl-1-[3-methyl4-(4,4,4-trifluoro-3-methoxymethoxy-3-trifluoromethyl-but-1-ynyl)-phenyl]-propyl}-2-methyl-phenoxy)-hexanoic acid ethyl ester

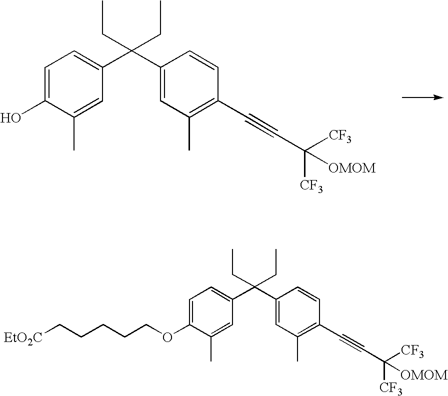

Using the same procedure as described for the preparation of Example 80-(1), the title compound was prepared from 4-{1-ethyl-1-[3-methyl4-(4,4,4-trifluoro-3-methoxymethoxy-3-trifluoromethyl-but-1-ynyl)-phenyl]-propyl}-2-methyl-phenol (compound prepared in Example 2-(2))and ethyl-6-bromohexanoate. The yield was 80%.

¹H-NMR (300 MHz, CDCl₃): 7.40 (d, 1H), 7.08 (bs, 1H), 7.00 (dd, 1H), 6.90 (dd, 1H), 6.85 (bd, 1H), 6.70 (d, 1H), 5.15 (s, 2H), 4.15 (q, 2H), 3.95 (m, 2H), 3.48 (s, 3H), 2.45-2.30 (m, 5H), 2.15 (s, 3H), 2.05 (q, 4H), 1.90-1.65 (m, 4H), 1.60-1.45 (m, 2H), 1.25 (t, 3H), 0.60 (t, 6H).

### (2) Preparation of 6-(4-{1-ethyl-1-[3-methyl-4-(4,4,4-trifluoro-3-hydroxy-3-trifluoromethyl-but-1-ynyl)-phenyl]-propyl}-2-methyl-phenoxy)-hexanoic acid ethyl ester

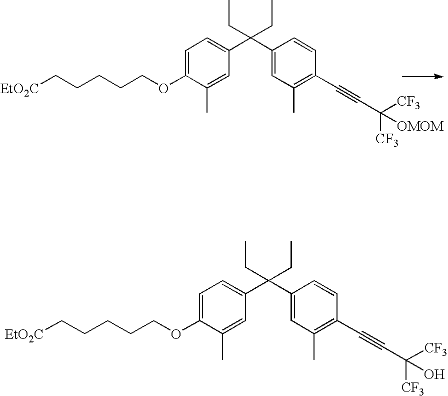

Using the same procedure as described for the preparation of Example 83-(2), the title compound was prepared from 6-(4-{1-ethyl-1-[3-methyl-4-(4,4,4-trifluoro-3-methoxymethoxy-3-trifluoromethyl-but-1-ynyl)-phenyl]-propyl}-2-methyl-phenoxy)-hexanoic acid ethyl ester (compound prepared in Example 91-(1)). The yield was 90%.

¹H-NMR (300 MHz, CDCl₃): 7.40 (d, 1H), 7.08 (bs,1H), 7.00 (dd, 1H), 6.90 (dd, 1H), 6.85 (bd, 1H), 6.70 (d, 1H), 4.15 (q, 2H), 3.95 (m, 3H), 2.45-2.30 (m, 5H), 2.15 (s, 3H), 2.05 (q, 4H), 1.90-1.75 (m, 4H), 1.30-1.20 (m, 5H), 0.60 (t, 6H).

### (3) Preparation of 6-(4-{1-ethyl-1-[3-methyl-4-(4,4,4-trifluoro-3-hydroxy-3-trifluoromethyl-but-1-ynyl)-phenyl]-propyl}-2-methyl-phenoxy)-hexanoic acid

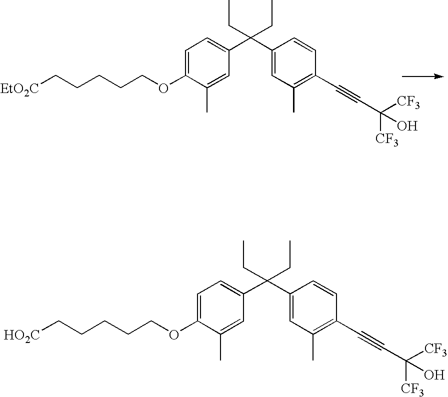

Using the same procedure as described for the preparation of Example 80-(2), the title compound was prepared from 6-(4-{1-ethyl-1-[3-methyl-4-(4,4,4-trifluoro-3-hydroxy-3-trifluoromethyl-but-1-ynyl)-phenyl]-propyl}-2-methyl-phenoxy)-hexanoic acid ethyl ester (compound prepared in Example 91-(2)). The yield was 72%.

¹H-NMR (300 MHz, CDCl₃): 7.40 (d, 1H), 7.08 (bs, 1H), 7.00 (dd, 1H), 6.90 (dd, 1H), 6.85 (bd, 1H), 6.70 (d, 1H), 3.95 (m, 2H), 3.80 (m, 1H), 2.50-2.35 (m, 5H), 2.15 (s, 3H), 2.05 (q, 4H), 1.90-1.70 (m, 4H), 1.65-1.50 (m, 2H), 0.60 (t, 6H); MS (ESI⁻): 571 ([M−H]⁻).

### Example 92

### Preparation of 6-(4-{1-Ethyl-1-[3-methyl-4-((E)4,4,4-trifluoro-3-hydroxy-3-trifluoromethyl-but-1-enyl)-phenyl]-propyl}-2-methyl-phenoxy)-hexanoic acid

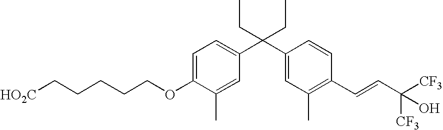

### (1) Preparation of 6-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxy)-hexanoic acid ethyl ester

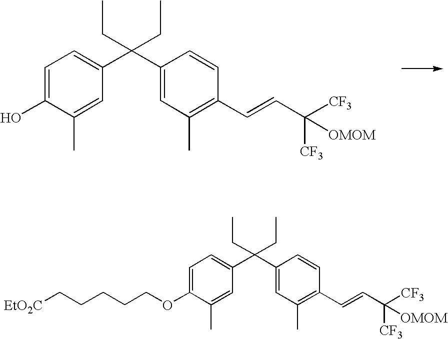

To a stirred solution of 4-{1-ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in Example 30-(1)) (143 mg, 0.28 mmol) and ethyl-6-bromohexanoate (0.15 ml, 0.85 mmol) in anhydrous DMF (1.4 ml) was added K₂CO₃ (116 mg, 0.84 mmol) under N₂ atmosphere at room temperature and the mixture was stirred at 110 degrees C. for 5 h. The reaction mixture was poured into water and the products were extracted with AcOEt. The extracts were washed with water and brine, dried over Na₂SO₄, filtered and concentrated under reduced pressure. The obtained residue was chromatographed on silica gel with n-Hexane:EtOAc (5:1) to give the title compound (160 mg, 88%) as colorless sticky oil.

¹H-NMR (300 MHz, CDCl₃): 7.50 (m, 2H), 7.15 (m, 2H), 7.00-6.85 (m, 2H), 6.70 (d, 1H), 6.08 (d, 1H), 4.98 (s, 2H), 4.15 (q, 2H), 3.95 (t, 2H), 3.50 (s, 3H), 2.40-2.35 (m, 6H), 2.15-2.00 (m, 4H), 1.90-1.50 (m, 8H), 1.25 (t, 3H), 0.60 (t, 6H).

### (2) Preparation of 6-(4-{1-ethyl-1-[3-methyl4-((E)-4,4,4-trifluoro-3-hydroxy-3-trifluoromethyl-but-1-enyl)-phenyl]-propyl}-2-methyl-phenoxy)-hexanoic acid ethyl ester

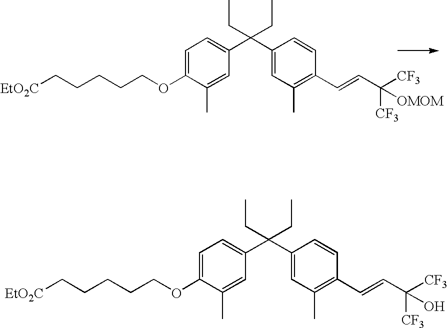

To a stirred solution of 6-(4-{1-ethyl-1-[3-methyl-4-((E)-4,4,4-trifluoro-3-methoxymethoxy-3-trifluoromethyl-but-1-enyl)-phenyl]-propyl}-2-methyl-phenoxy)-hexanoic acid ethyl ester (compound prepared in Example 92-(1)) (160 mg, 0.25 mmol) in of EtOH (2.0 ml) was added CBr₄ (13.6 mg, 10 mol %) under N₂ atmosphere at room temperature and the mixture was stirred at reflux temperature for 2.5 h. The reaction mixture was cooled to room temperature, poured into water and the products were extracted with EtOAc. The extracts were washed with brine, dried over Na₂SO₄, filtered and concentrated under reduced pressure. The obtained residue was chromatographed on silica gel with EtOAc-n-Hexane (1:10) to give the title compound (122 mg, 73%) as colorless sticky oil.

¹H-NMR (300 MHz, CDCl₃): 7.35 (m, 2H), 7.05 (m, 2H), 6.95-6.85 (m, 2H), 6.68 (d, 1H), 6.10 (d, 1H), 4.15 (q, 2H), 3.95 (t, 2H), 3.20 (bs, 1H), 2.40-2.30 (m, 5H), 2.15 (s, 3H), 2.05 (q, 4H), 1.85-1.65 (m, 4H), 1.60-1.45 (m, 2H), 1.25 (t, 3H), 0.60 (t, 6H).

### (3) Preparation of 6-(4-{1-ethyl-1-[3-methyl4-((E)-4,4,4-trifluoro-3-hydroxy-3-trifluoromethyl-but-1-enyl)-phenyl]-propyl}-2-methyl-phenoxy)-hexanoic acid

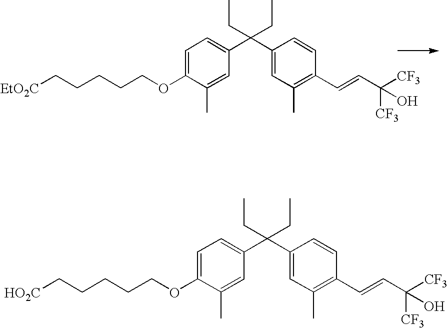

To a stirred solution of 6-(4-{1-ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid ethyl ester (compound prepared in Example 92-(2)) (122 mg, 0.20 mmol) in EtOH/Water (4/1 v/v, 2.5 ml) was added KOH (23.0 mg, 0.41 mmol) and the mixture was stirred at room temperature for 4 h. The reaction mixture was poured into 0.1N HCl and the products were extracted with EtOAc. The extracts were washed with brine, dried over Na₂SO₄, filtered and concentrated under reduced pressure. The obtained residue was chromatographed on silica gel with EtOAc-n-Hexane (1:2) to give the title compound (90 mg, 78%) as colorless sticky oil.

¹H-NMR (300 MHz, CDCl₃): 7.35 (m, 2H), 7.05 (m, 2H), 6.95-6.85 (m, 2H), 6.68 (d, 1H), 6.10 (d, 1H), 3.95 (t, 2H), 2.40-2.30 (m, 5H), 2.15 (s, 3H), 2.05 (q, 4H), 1.85-1.65 (m, 4H), 1.60-1.45 (m, 2H), 0.60 (t, 6H); MS (ESI⁻): 573 ([M−H]⁻).

### Example 93

### Preparation of 6-(4-{1-ethyl-1-[4-(3-hydroxy-4,4-dimethyl-pentyl)-3-methyl-phenyl]-propyl}-2-methyl-phenoxy)-hexanoic acid

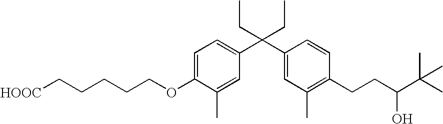

### (1) Preparation of 6-(4-{1-ethyl-1-[4-(3-hydroxy-4,4-dimethyl-pentyl)-3-methyl-phenyl]-propyl}-2-methyl-phenoxy)-hexanoic acid ethyl ester

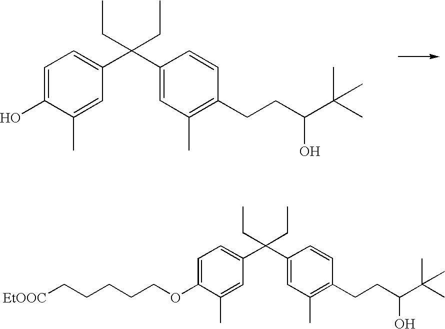

To a solution of 4-{1-ethyl-1-[4-(3-hydroxy-4,4-dimethyl-pentyl)-3-methyl-phenyl]-propyl}-2-methyl-phenol (compound prepared in Example 18) (200 mg, 0.52 mmol) in DMF (5 ml) were added K₂CO₃ (144 mg, 1.04 mmol) and 6-bromohexanoic acid ethyl ester (0.184 ml, 1.04 mmol) and the mixture was stirred at 50 degrees C. for 13 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:10) to give the title compound (214 mg, 78%).

¹H-NMR (300 MHz, CDCl₃): 0.6 (t, 6H), 0.89 (s, 9H), 1.29 (m, 3H), 1.52 (m, 3H), 1.81 (m, 5H), 2.05 (q, 4H), 2.16 (s,3H), 2.26 (s,3H), 2.35 (t, 2H), 2.56 (m, 1H), 2.85 (m, 1H), 3.26 (m, 1H), 3.92 (t, 2H), 4.12 (q, 2H), 6.66 (d, 1H), 6.90 (m, 4H), 7.02 (d, 1H).

### (2) Preparation of 6-(4-{1-ethyl-1-[4-(3-hydroxy-4,4-dimethyl-pentyl)-3-methyl-phenyl]-propyl}-2-methyl-phenoxy)-hexanoic acid

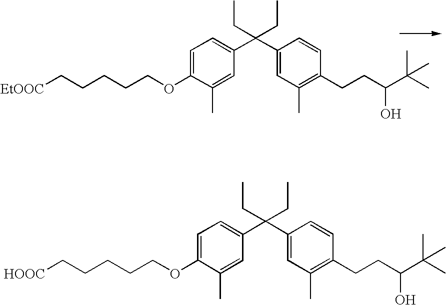

To a stirred solution of 6-(4-{1-ethyl-1-[4-(3-hydroxy-4,4-dimethyl-pentyl)-3-methyl-phenyl]-propyl}-2-methyl-phenoxy)-hexanoic acid ethyl ester (compound prepared in Example 93-(1)) (214 mg, 0.408 mmol) in MeOH (3 ml) was added 1N—KOH (1.22 ml, 1.22 mmol) and the mixture was stirred at room temperature for 5 h. The reaction mixture was poured into 1 N-HCl and the products were extracted with EtOAc. The extracts were washed with brine, dried over MgSO₄, filtered and concentrated under reduced pressure. The obtained residue was chromatographed on silica gel with EtOAc-n-Hexane (1:3) to give the title compound (144 mg, 71%).

¹H-NMR (300 MHz, CDCl₃): 0.6 (t, 6H), 0.89 (s, 9H), 1.52 (m, 3H), 1.81 (m, 5H), 2.05 (q, 4H), 2.16 (s,3H), 2.26 (s,3H), 2.39 (t, 2H), 2.56 (m, 1H), 2.85 (m, 1H), 3.26 (m,1H), 3.92 (t, 2H), 6.66 (d, 1H), 6.90 (m, 4H), 7.02 (d, 1H); MS (ESI⁻): 495 ([M−H]⁻).

### Example 94

### Preparation of 6-(4-{1-ethyl-1-[4-(1-hydroxy-cyclopentylethynyl)-3-methyl-phenyl]-propyl}-2-methyl-phenoxy)-hexanoic acid

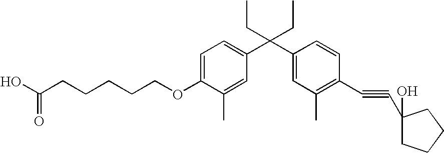

### (1) Preparation of 6-(4-{1-ethyl-1-[4-(1-hydroxy-cyclopentylethynyl)-3-methyl-phenyl]-propyl}-2-methyl-phenoxy)-hexanoic acid ethyl ester

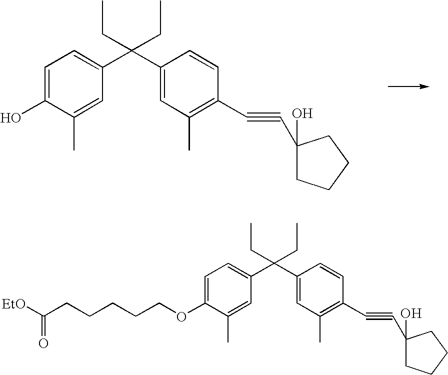

To a solution of 4-{1-ethyl-1-[4-(1-hydroxy-cyclopentylethynyl)-3-methyl-phenyl]-propyl}-2-methyl-phenol (compound prepared in Example 7) (114 mg, 0.303 mmol) in DMF (5 ml) were added K₂CO₃ (126 mg, 0.908 mmol) and 6-bromo-hexanoic acid ethyl ester (135 mg, 0.606 mmol) and the mixture was stirred at 90 degrees C. for 6 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:6) to give the title compound (71 mg, 45%).

¹H-NMR (300 MHz, CDCl₃): 0.59 (t, 6H), 1.25 (t, 3H), 1.31-1.79 (m, 10H), 1.85 (m, 4H), 2.03 (q, 4H), 2.14 (s, 3H), 2.30 (t, 2H), 2.36 (s, 3H), 3.99 (t, 2H), 4.18 (q, 2H), 6.68 (d, 1H), 6.84 (m, 2H), 6.91 (d, 1H), 7.00 (s, 1H), 7.27 (d, 1H).

### (2) Preparation of 6-(4-1-ethyl-1-[4-(1-hydroxy-cyclopentylethynyl)-3-methyl-phenyl]-propyl}-2-methyl-phenoxy)-hexanoic acid

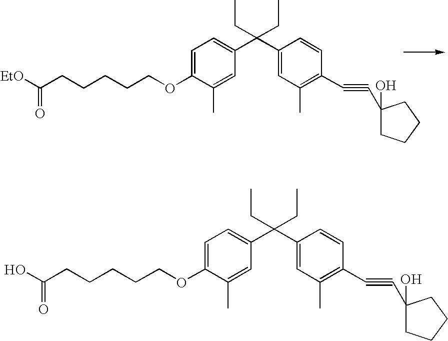

To a stirred solution of 6-(4-{1-ethyl-1-[4-(1-hydroxy-cyclopentylethynyl)-3-methyl-phenyl]-propyl}-2-methyl-phenoxy)-hexanoic acid ethyl ester (compound prepared in Example 94-(1)) (71 mg, 0.137 mmol) in MeOH (1.4 ml) was added 1N—KOH (0.41 ml, 0.411 mmol) and the mixture was stirred at room temperature for 18 h. The reaction mixture was poured into 1 N—HCl and the products were extracted with EtOAc. The extracts were washed with brine, dried over MgSO₄, filtered and concentrated under reduced pressure. The obtained residue was chromatographed on silica gel with EtOAc-n-Hexane (1:2) to give the title compound (56.6 mg, 84%).

¹H-NMR (300 MHz, CDCl₃): 0.60 (t, 6H), 1.32-1.79 (m, 14H), 2.03 (q, 4H), 2.14 (s, 3H), 2.37 (s, 3H), 2.40 (t, 2H), 3.91 (t, 2H), 6.68 (d, 1H), 6.84 (m, 2), 6.91 (d, 1H), 7.00 (s, 1H), 7.27 (d, 1H); MS (ESI−): 489 ([M−H]⁻).

### Example 95

### Preparation of 6-[4-(1-ethyl-1-{4-[(E)-2-(1-hydroxy-cyclopentyl)-vinyl]-3-methyl-phenyl}-propyl)-2-methyl-phenoxy]-hexanoic acid

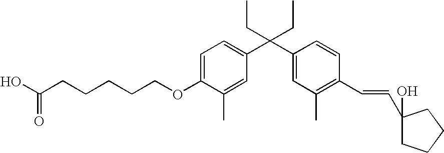

### (1) Preparation of 6-[4-(1-ethyl-1-{4-[(E)-2-(1-hydroxy-cyclopentyl)-vinyl]-3-methyl-phenyl}-propyl)-2-methyl-phenoxy]-hexanoic acid ethyl ester

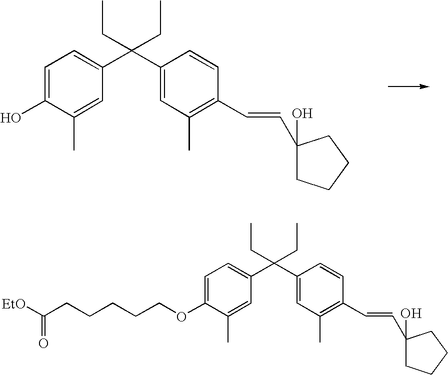

To a solution of 4-(1-ethyl-1-{4-[(E)-2-(1-hydroxy-cyclopentyl)-vinyl]-3-methyl-phenyl}-propyl)-2-methyl-phenol (compound prepared in Example 10) (110 mg, 0.291 mmol) in DMF (5 ml) were added K₂CO₃ (120 mg, 0.872 mmol) and 6-bromo-hexanoic acid ethyl ester (130 mg, 0.582 mmol) and the mixture was stirred at 90 degrees C. for 6 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:7) to give the title compound (73 mg, 48%).

¹H-NMR (300 MHz, CDCl₃): 0.58 (t, 6H), 1.25 (t, 3H), 1.39-1.88 (m, 14H), 2.03 (q, 4H), 2.13 (s, 3H), 2.30 (t, 2H), 2.38 (s, 3H), 3.92 (t, 2H), 4.12 (q, 2H), 6.21 (d, 1H), 6.69 (d, 1H), 6.84 (d, 1H), 6.87-7.00 (m, 4H), 7.35 (d, 1H).

### (2) Preparation of 6-[4-(1-ethyl-1-{4-[(E)-2-(1-hydroxy-cyclopentyl)-vinyl]-3-methyl-phenyl}-propyl)-2-methyl-phenoxy]-hexanoic acid

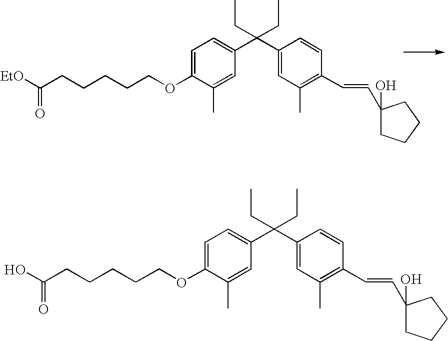

To a stirred solution of 6-[4-(1-ethyl-1-{4-[(E)-2-(1-hydroxy-cyclopentyl)-vinyl]-3-methyl-phenyl}-propyl)-2-methyl-phenoxy]-hexanoic acid ethyl ester (compound prepared in Example 95-(1)) (73 mg, 0.140 mmol) in MeOH (1.4 ml) was added 1N—KOH (0.42 ml, 0.420 mmol) and the mixture was stirred at room temperature for 18 h. The reaction mixture was poured into 1N—HCl and the products were extracted with EtOAc. The extracts were washed with brine, dried over MgSO₄, filtered and concentrated under reduced pressure. The obtained residue was chromatographed on silica gel with EtOAc-n-Hexane (1:2) to give the title compound (60 mg, 87%).

¹H-NMR (300 MHz, CDCl₃): 0.60 (t, 6H), 1.32-1.79 (m, 14H), 2.03 (q, 4H), 2.14 (s, 3H), 2.31 (s, 3H), 2.40 (t, 2H), 3.93 (t, 2H), 6.25 (d, 1H), 6.65 (d, 1H), 6.81-6.96 (m, 5H), 7.31 (d, 1H); MS(ESI−) : 491 ([M−H]⁻).

### Example 96

### Preparation of 6-(4-{1-ethyl-I -[4-(1-hydroxy-cyclohexylethynyl)-3-methyl-phenyl]-propyl}-2-methyl-phenoxy)-hexanoic acid

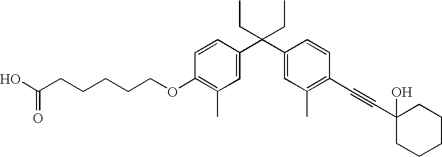

### (1) Preparation of 6-(4-{1-ethyl-1-[4-(1-hydroxy-cyclohexylethynyl)-3-methyl-phenyl]-propyl}-2-methyl-phenoxy)-hexanoic acid ethyl ester

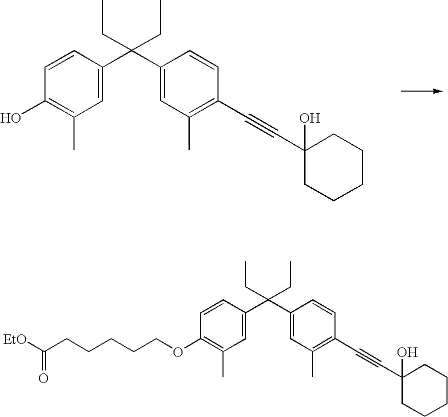

To a solution of 4-{1-ethyl-1-[4-(1-hydroxy-cyclohexylethynyl)-3-methyl-phenyl]-propyl}-2-methyl-phenol (compound prepared in Example 8) (110 mg, 0.28 mmol) in DMF (3 ml) were added K₂CO₃ (116 mg, 0.84 mmol) and 6-bromo-hexanoic acid ethyl ester (0.1 ml, 0.56 mmol) and the mixture was stirred at 90 degrees C. for 13 h. The reaction mixture was cooled to room temperature, poured into saturated NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:6) to give the title compound (133 mg, 89%).

¹H-NMR (300 MHz, CDCl₃): 0.59 (t, 6H),1.25 (t, 3H), 1.49-1.82 (m, 16H), 2.03 (q, 4H), 2.14 (s, 3H), 2.33 (t, 2H), 2.38 (s, 3H), 3.92 (t, 2H), 4.12 (q, 2H), 6.66 (d, 1H), 6.84-7.01 (m, 4H), 7.27 (d, 1H).

### (2) Preparation of 6-(4-{1-ethyl-1-[4-(1-hydroxy-cyclohexylethynyl)-3-methyl-phenyl]-propyl}-2-methyl-phenoxy)-hexanoic acid

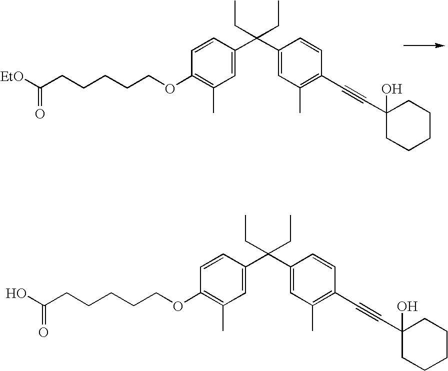

To a stirred solution of 6-(4-(1-ethyl-1-[4-(1-hydroxy-cyclohexylethynyl)-3-methyl-phenyl]-propyl}-2-methyl-phenoxy)-hexanoic acid ethyl ester (compound prepared in Example 96-(1)) (133 mg, 0.25 mmol) in MeOH (2 ml) was added 1N—KOH (0.75 ml, 0.75 mmol) and the mixture was stirred at room temperature for 13 h. The reaction mixture was poured into saturated NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:2) to give the title compound (95 mg, 75%).

¹H-NMR (300 MHz, CDCl₃): 0.59 (t, 6H), 1.43-1.82 (m, 16H), 2.03 (q, 4H), 2.14 (s, 3H), 2.38 (s, 3H), 2.40 (t, 2H), 3.93 (t, 2H), 6.66 (d, 1H), 6.84-7.01 (m, 4H), 7.27 (d, 1H); MS (ESI−) : 503 ([M−H]⁻).

### Example 97

### Preparation of 6-[4-(1-ethyl-1-{4-[(E)-2-(1-hydroxy-cyclohexyl)-vinyl]-3-methyl-phenyl}-propyl)-2-methyl-phenoxy]-hexanoic acid

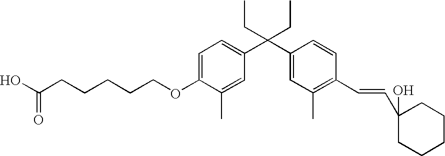

### (1) Preparation of 6-[4-(1-ethyl-1-{4-[(E)-2-(1-hydroxy-cyclohexyl)-vinyl]-3-methyl-phenyl}-propyl)-2-methyl-phenoxy]-hexanoic acid ethyl ester

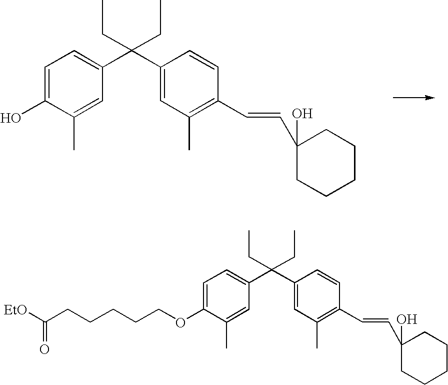

To a solution of 4-(1-ethyl-1-{4-[(E)-2-(1-hydroxy-cyclohexyl)-vinyl]-3-methyl-phenyl}-propyl)-2-methyl-phenol (compound prepared in Example 11) (96.6 mg, 0.246 mmol) in DMF (3 ml) were added K₂CO₃ (102 mg, 0.738 mmol) and 6-bromo-hexanoic acid ethyl ester (110 mg, 0.492 mmol) and the mixture was stirred at 90 degrees C. for 12 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:6) to give the title compound (112 mg, 85%).

¹H-NMR (300 MHz, CDCl₃): 0.59 (t, 6H), 1.25 (t, 3H), 1.31-1.79 (m, 12H), 1.85 (m, 4H), 2.03 (q, 4H), 2.14 (s, 3H), 2.30 (s, 3H), 2.38 (t, 2H), 3.92 (t, 2H), 4.3 (q, 2H), 6.19 (d, 1H), 6.65 (d, 1H), 6.82 (d, 1H), 6.89-7.00 (m, 4H), 7.32 (d, 1H); MS (ESI+): 557 ([M+Na]⁺).

### (2) Preparation of 6-[4-(1-ethyl-1-{4-[(E)-2-(l-hydroxy-cyclohexyl)-vinyl]-3-methyl-phenyl}-propyl)-2-methyl-phenoxy]-hexanoic acid

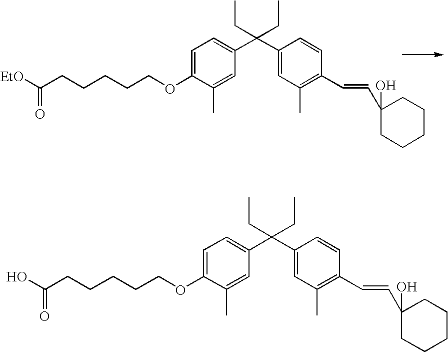

To a stirred solution of 6-[4-(1-ethyl-1-{4-[(E)-2-(1-hydroxy-cyclohexyl)-vinyl]-3-methyl-phenyl}-propyl)-2-methyl-phenoxy]-hexanoic acid ethyl ester (compound prepared in Example 97-(1)) (112 mg, 0.209 mmol) in MeOH (2.1 ml) was added 1N—KOH (0.63 ml, 0.63 mmol) and the mixture was stirred at room temperature for 12 h. The reaction mixture was poured into saturated NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:2) to give the title compound (76 mg, 71%).

¹H-NMR (300 MHz, CDCl₃): 0.60 (t, 6H), 1.32-1.79 (m, 16H), 2.03 (q, 4H), 2.14 (s, 3H), 2.30 (s, 3H), 2.40 (t, 2H), 3.93 (t, 2H), 6.19 (d, 1H), 6.65 (d, 1H), 6.80 (d, 1H), 6.9-6.95 (m, 4H), 7.30 (d, 1H); MS (ESI−): 505 ([M−H]⁻).

### Example 98

### Preparation of 7-(4-{1-ethyl-1-[4-(3-hydroxy-4,4-dimethyl-pentyl)-3-methyl-phenyl]-propyl}-2-methyl-phenoxy)-heptanoic acid

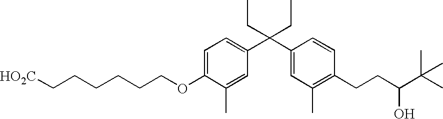

### (1) Preparation of 7-(4-1-ethyl-1-[4-(3-hydroxy-4,4-dimethyl-pentyl)-3-methyl-phenyl]-propyl}-2-methyl-phenoxy)-heptanoic acid ethyl ester

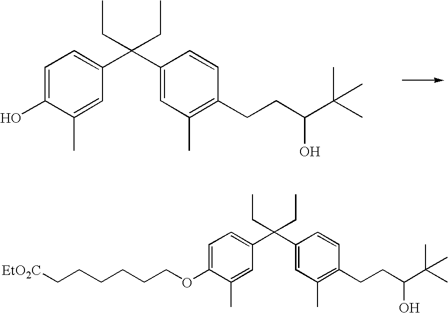

To a solution of 4-{1-ethyl-1-[4-(3-hydroxy-4,4-dimethyl-pentyl)-3-methyl-phenyl]-propyl}-2-methyl-phenol (compound prepared in Example 18) (200 mg, 0.52 mmol) in DMF (5 ml) were added K₂CO₃ (144 mg, 1.04 mmol) and 7-bromoheptanoic acid ethyl ester (0.202 ml, 1.04 mmol) and the mixture was stirred at 50 degrees C. for 13 h. The reaction mixture was cooled to room temperature, poured into water and the products were extracted with EtOAc. The extracts were washed with brine, dried over MgSO₄, filtered and concentrated under reduced pressure. The obtained residue was chromatographed on silica gel with EtOAc-n-Hexane (1:10) to give the title compound (231 mg, 83%).

¹H-NMR (300 MHz, CDCl₃): 0.6 (t, 6H), 0.89 (s, 9H), 1.29 (m, 3H), 1.45 (m, 5H), 1.69 (m, 2H), 1.81 (m, 3H), 2.05 (q, 4H), 2.16 (s,3H), 2.26 (s,3H), 2.31 (t, 2H), 2.56 (m, 1H), 2.85 (m, 1H), 3.26 (m, 1H), 3.92 (t, 2H), 4.12 (q, 2H), 6.66 (d, 1H), 6.90 (m, 4H), 7.02 (d, 1H).

### (2) Preparation of 7-(4-{1-ethyl-1-[4-(3-hydroxy-4,4-dimethyl-pentyl)-3-methyl-phenyl]-propyl}-2-methyl-phenoxy)-heptanoic acid

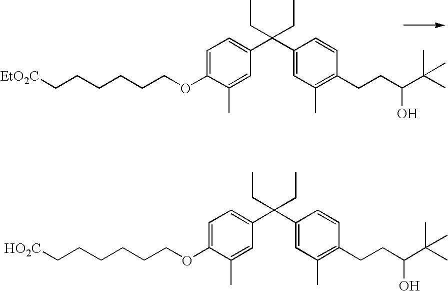

To a stirred solution of 7-(4-{1-ethyl-1-[4-(3-hydroxy-4,4-dimethyl-pentyl)-3-methyl-phenyl]-propyl}-2-methyl-phenoxy)-heptanoic acid ethyl ester (compound prepared in Example 98-(1)) (231 mg, 0.429 mmol) in MeOH (3 ml) was added 1N—KOH (1.29 ml, 1.29 mmol) and the mixture was stirred at room temperature for 5 h. The reaction mixture was poured into 1N—HCl and the products were extracted with EtOAc. The extracts were washed with brine, dried over MgSO₄, filtered and concentrated under reduced pressure. The obtained residue was chromatographed on silica gel with EtOAc-n-Hexane (1:3) to give the title compound (153 mg, 70%).

¹H-NMR (300 MHz, CDCl₃): 0.6 (t, 6H), 0.89 (s, 9H), 1.45 (m, 5H), 1.69 (m, 2H), 1.81 (m, 3H), 2.05 (q, 4H), 2.16 (s,3H), 2.26 (s,3H), 2.38 (t, 2H), 2.56 (m, 1H), 2.85 (m, 1H), 3.26 (m, 1H), 3.91 (t, 2H), 6.66 (d, 1H), 6.90 (m, 4H), 7.02 (d, 1H); MS (ESI−): 509 ([M−H]⁻).

### Example 99

### Preparation of 1-(4-{1-[4-(2-amino-ethoxy)-3-methyl-phenyl]-1-ethyl-propyl}-2-methyl-phenyl)4,4-dimethyl-pentan-3-ol

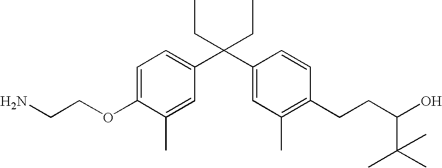

### Preparation of 1-(4-{1-[4-(2-amino-ethoxy)-3-methyl-phenyl]-1-ethyl-propyl}-2-methyl-phenyl)-4,4-dimethyl-pentan-3-ol

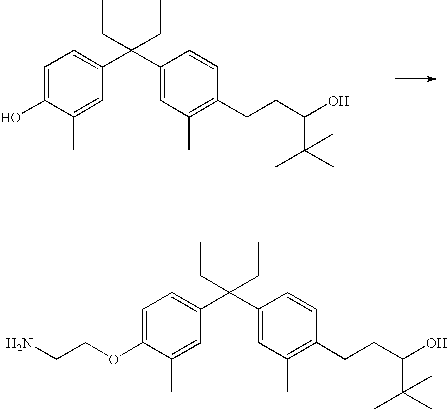

To a solution of 4-{1-ethyl-1-[4-(3-hydroxy-4,4-dimethyl-pentyl)-3-methyl-phenyl]-propyl}-2-methyl-phenol (compound prepared in Example 18) (100 mg, 0.261 mmol) in THF-DMF(5:1, 0.45 ml) was added NaH (46 mg, 1.17 mmol) at 0 degrees C. and the mixture was stirred at room temperature for 15 min. Then bromoethylamine HBr salt (79 mg, 0.391 mmol) was added and the mixture was stirred at 55 degrees C. for 1.5 h. The reaction mixture was cooled to 0 degrees C., neutralized to pH7 with sat. NH₄Cl aq. The reaction mixture was poured into water and the products were extracted with EtOAc. The extracts were washed with brine, dried over Na₂SO₄, filtered and concentrated under reduced pressure. The obtained residue was chromatographed on amino functionalized silica gel with EtOAc-n-Hexane (3:1) to give the title compound (55 mg, 50%).

¹H-NMR (300 MHz, CDCl₃): 0.59 (t, 6H), 0.89 (s, 9H), 1.43-1.57 (m, 1H), 1.74-1.84 (m, 1H), 2.03 (q, 4H), 2.17(s, 3H), 2.25 (s, 3H), 2.50-2.60 (m, 1H), 2.81-2.91 (m, 1H), 3.07 (t, 2H), 3.24 (d, 1H), 3.96 (t, 2H), 6.68 (d, 1H), 6.90-6.96 (m, 4H), 7.01 (d, 1H); MS (ESI+) :426 ([M+H]⁺).

### Example 100

### Preparation of 1-(4-{1-[4-(3-amino-propoxy)-3-methyl-phenyl]-1-ethyl-propyl}-2-methyl-phenyl)-4,4-dimethyl-pentan-3-ol

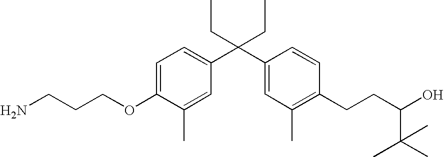

### (1) Preparation of 2-[3-(4-{1-ethyl-1-[4-(3-hydroxy-4,4-dimethyl-pentyl)-3-methyl-phenyl]-propyl}-2-methyl-phenoxy)-propyl]-isoindole-1,3-dione

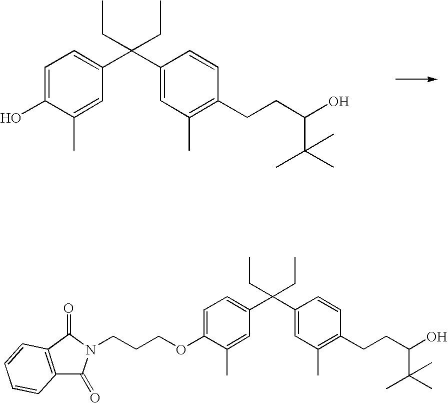

To a solution of 4-{1-ethyl-1-[4-(3-hydroxy-4,4-dimethyl-pentyl)-3-methyl-phenyl]-propyl}-2-methyl-phenol (compound prepared in Example 18) (340 mg, 0.915 mmol) in acetonitrile (9 ml) were added K₂CO₃ (253 mg, 1.83 mmol) and N-(3-bromopropyl)phthalimide (491 mg, 1.83 mmol) and the mixture was stirred at reflux temperature for 5 h. The reaction mixture was cooled to room temperature, concentrated under reduced pressure. The residue was diluted with EtOAc and washed with water and brine, dried over Na₂SO₄, filtered and concentrated under reduced pressure. The obtained residue was chromatographed on silica gel with EtOAc-n-Hexane (1:6) to give the title compound (460 mg, 88%).

¹H-NMR (300 MHz, CDCl₃): 0.58 (t, 6H), 0.89 (s, 9H), 1.37 (d, 1H), 1.42-1.53 (m, 1H), 1.78-1.84 (m,1H), 2.04 (q, 4H), 2.11(s, 3H), 2.14-2.21 (m, 2H), 2.25 (s, 3H), 2.50-2.60 (m, 1H), 2.77-2.92 (m, 1H), 3.21-3.27 (dd, 1H), 3.91 (t, 2H), 4.00 (t, 2H), 6.66 (d, 1H), 6.88-6.92 (m, 4H), 7.00 (d, 1H), 7.68-7.71 (m, 2H), 7.82-7.85 (m, 2H); MS (ESI+): 587 ([M+NH₄]⁺).

### (2) Preparation of 1-(4-{1-[4-(3-amino-propoxy)-3-methyl-phenyl]-1-ethyl-propyl}-2-methyl-phenyl)-4,4-dimethyl-pentan-3-ol

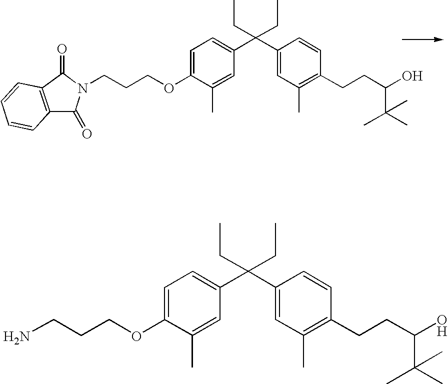

To a solution of 2-[3-(4-{1-ethyl-1-[4-(3-hydroxy-4,4-dimethyl-pentyl)-3-methyl-phenyl]-propyl}-2-methyl-phenoxy)-propyl]-isoindole-1,3-dione (compound prepared in Example 100-(1)) (460 mg, 0.807 mmol) in EtOH (6 ml) was added hydrazine hydrate(0.4 ml, 8.07 mmol) and the mixture was stirred at reflux temperature for 1 h. The reaction mixture was cooled to room temperature, concentrated under reduced pressure. The obtained residue Was diluted with EtOAc, and filtered. The filtrate was concentrated, and purified by amino functionalized silica gel chromatography (EtOAc-n-Hexane=1:1 to EtOAc) to give the title compound (264 mg, 74%).

¹H-NMR (300 MHz, CDCl₃): 0.59 (t, 6H), 0.89 (s, 9H), 1.43-1.57 (m, 1H), 1.74-1.84 (m, 1H), 1.92 (q, 2H), 2.03 (q, 4H), 2.15 (s, 3H), 2.25 (s, 3H), 2.49-2.62 (m, 1H), 2.81-2.89 (m, 1H), 2.92 (t, 2H), 3.23 (dd, 1H), 4.01 (t, 2H), 6.68 (d, 1H), 6.90-6.96 (m, 4H), 7.01 (d, 1H); MS (ESI+): 440 ([M+H]⁺).

### Example 101

### Preparation of 1-(4-{1-[4-(4-amino-butoxy)-3-methyl-phenyl]-1-ethyl-propyl}-2-methyl-phenyl)4,4-dimethyl-pentan-3-ol

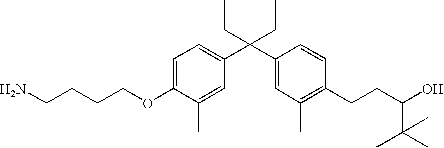

### (1) Preparation of 2-[4-(4-{1-ethyl-1-[4-(3-hydroxy-4,4-dimethyl-pentyl)-3-methyl-phenyl]-propyl}-2-methyl-phenoxy)-butyl]-isoindole-1,3-dione

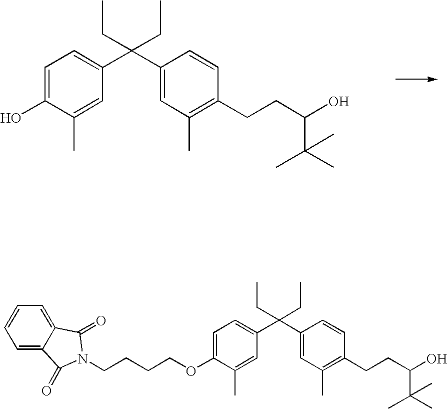

Using the same procedure as described for the preparation of Example 100-(1), the title compound was prepared from 4-{1-Ethyl-1-[4-(3-hydroxy-4,4-dimethyl-pentyl)-3-methyl-phenyl]-propyl}-2-methyl-phenol (compound prepared in Example 18) and N-(4-bromobutyl)phthalimide. The yield was 97%.

¹H-NMR (300 MHz, CDCl₃): 0.58 (t, 6H), 0.88 (s, 9H), 1.39 (d, 1H), 1.44-1.55 (m, 1H), 1.74-1.96 (m, 5H), 2.03 (q, 4H), 2.14 (s, 3H), 2.25 (s, 3H), 2.49-2.62 (m, 1H), 2.79-2.92 (m, 1H), 3.23 (dd, 1H), 3.77 (t, 2H), 3.96 (t, 2H), 6.64 (d, 1H), 6.87-6.95 (m, 4H), 7.00 (d, 1H), 7.65-7.75 (m, 2H), 7.78-7.87 (m, 2H); MS (ESI+): 601 ([M+NH₄]⁺).

### (2) Preparation of 1-(4-{1-[4-(4-amino-butoxy)-3-methyl-phenyl]-1-ethyl-propyl}-2-methyl-phenyl)-4,4-dimethyl-pentan-3-ol

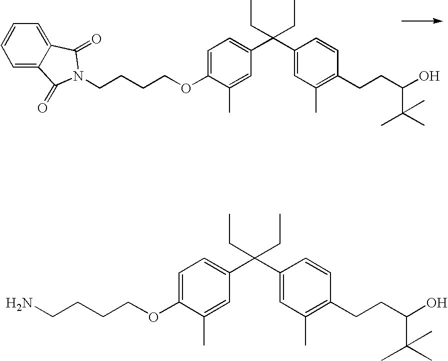

Using the same procedure as described for the preparation of Example 100-(2), the title compound was prepared from 2-[4-(4-{1-ethyl-1-[4-(3-hydroxy-4,4-dimethyl-pentyl)-3-methyl-phenyl]-propyl}-2-methyl-phenoxy)-butyl]-isoindole-1,3-dione (compound prepared in Example 101-(1)). The yield was 87%.

¹H-NMR (300 MHz, CDCl₃): 0.59 (t, 6H), 0.89 (s, 9H), 1.44-1.59 (m, 1H), 1.59-1.70 (m, 2H), 1.72-1.88 (m, 3H), 2.03 (q, 4H), 2.15 (s, 3H), 2.25 (s, 3H), 2.48-2.62 (m, 1H), 2.75 (t, 2H), 2.80-2.92 (m,1H), 3.24 (dd,1H), 3.94 (t, 2H), 6.64 (d, 1H), 6.88-6.96 (m, 4H), 7.01 (d, 1H); MS (ESI+): 454 ([M+H]⁺).

### Example 102

### Preparation of 1-(4-{1-[4-(5-amino-pentyloxy)-3-methyl-phenyl]-1-ethyl-propyl}-2-methyl-phenyl)-4,4-dimethyl-pentan-3-ol

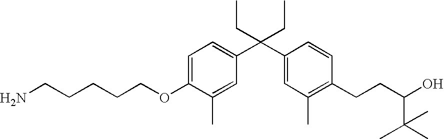

### (1)Preparation of 2-[5-(4-{1-ethyl-1-[4-(3-hydroxy-4,4-dimethyl-pentyl)-3-methyl-phenyl]-propyl}-2-methyl-phenoxy)-pentyl]-isoindole-1,3-dione

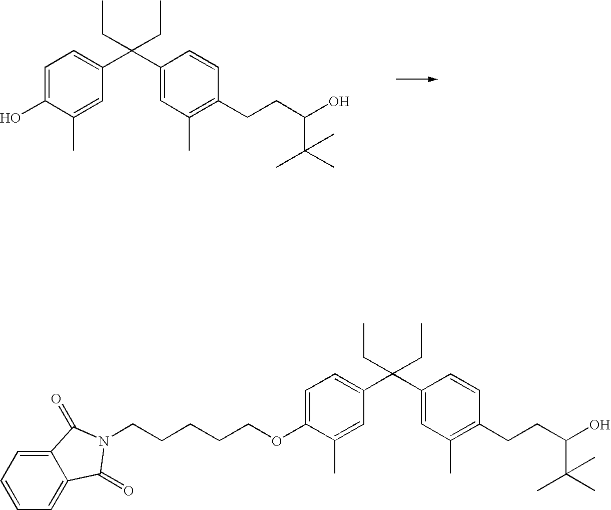

Using the same procedure as described for the preparation of Example 100-(1), the title compound was prepared from 4-{1-ethyl-1-[4-(3-hydroxy-4,4-dimethyl-pentyl)-3-methyl-phenyl]-propyl}-2-methyl-phenol (compound prepared in Example 18) and N-(5-bromopentyl)phthalimide. The yield was 99%.

¹H-NMR (300 MHz, CDCl₃): 0.59 (t, 6H), 0.89 (s, 9H), 1.38 (d, 1H), 1.45-1.58 (m, 3H), 1.71-1.87 (m, 5H), 2.06 (q, 4H), 2.11 (s, 3H), 2.25 (s, 3H), 2.50-2.61 (m, 1H), 2.80-2.90 (m, 1H), 3.24 (dd, 1H), 3.72 (t, 2H), 3.91 (t, 2H), 6.64 (d, 1H), 6.85-6.95 (m, 4H), 7.01 (d, 1H), 7.66-7.72 (m, 2H), 7.78-7.88 (m, 2H); MS (ESI+): 615 ([M+NH₄]⁺).

### (2) Preparation of 1-(4-{1-[4-(5-amino-pentyloxy)-3-methyl-phenyl]-1-ethyl-propyl}-2-methyl-phenyl)4,4-dimethyl-pentan-3-ol

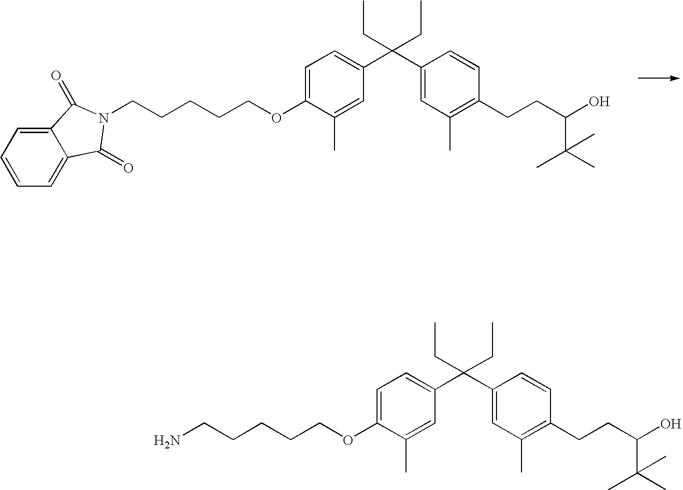

Using the same procedure as described for the preparation of Example 100-(2), the title compound was prepared from 2-[5-(4-{1-ethyl-1-[4-(3-hydroxy-4,4-dimethyl-pentyl)-3-methyl-phenyl]-propyl}-2-methyl-phenoxy)-pentyl]-isoindole-1,3-dione (compound prepared in Example 102-(1)). The yield was 87%.

¹H-NMR (300 MHz, CDCl₃): 0.59 (t, 6H), 0.89 (s, 9H), 1.45-1.56 (m, 5H), 1.74˜1.84 (m, 3H), 2.03 (q, 4H), 2.15 (s, 3H), 2.25 (s, 3H), 2.50-2.60 (m, 1H), 2.72 (t, 2H), 2.80-2.90 (m, 1H), 3.24 (dd, 1H), 3.92 (t, 2H), 6.67 (d, 1H), 6.85-6.96 (m, 4H), 7.02 (d, 1H); MS (ESI+): 468 ([M+H]⁺).

### Example 103

### Preparation of 1-(4-{1-[4-(6-amino-hexyloxy)-3-methyl-phenyl]-1-ethyl-propyl}-2-methyl-phenyl)-4,4-dimethyl-pentan-3-ol

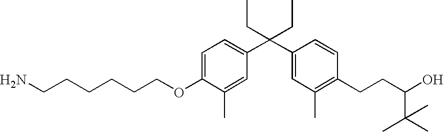

### (1) Preparation of 2-[6-(4-{1-ethyl-1-[4-(3-hydroxy-4,4-dimethyl-pentyl)-3-methyl-phenyl]-propyl}-2-methyl-phenoxy)-hexyl]-isoindole-1,3-dione

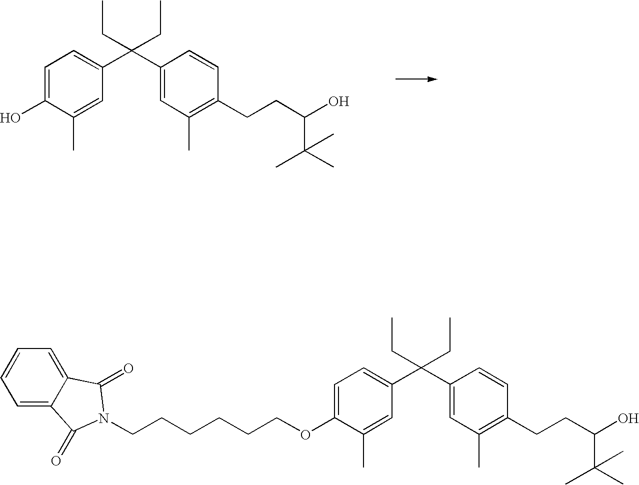

Using the same procedure as described for the preparation of Example 100-(1), the title compound was prepared from 4-{1-ethyl-1-[4-(3-hydroxy-4,4-dimethyl-pentyl)-3-methyl-phenyl]-propyl}-2-methyl-phenol (compound prepared in Example 18) and N-(6-bromohexyl)phthalimide. The yield was 99%.

¹H-NMR (300 MHz, CDCl₃): 0.59 (t, 6H), 0.89 (s, 9H), 1.36-1.56 (m, 5H), 1.65-1.85 (m, 5H), 2.03 (q, 4H), 2.13 (s, 3H), 2.25 (s, 3H), 2.50-2.60 (m, 1H), 2.81-2.90 (m, 1H), 3.24 (dd, 1H), 3.69 (t, 2H), 3.90 (t, 2H), 6.65 (d, 1H), 6.89-6.93 (m, 4H), 7.01 (d, 1H), 7.65-7.74 (m, 2H), 7.78-7.87 (m, 2H); MS (ESI+): 629 ([M+NH₄]⁺).

### (2) Preparation of 1-(4-{1-[4-(6-Amino-hexyloxy)-3-methyl-phenyl]-1-ethyl-propyl}-2-methyl-phenyl)4,4-dimethyl-pentan-3-ol

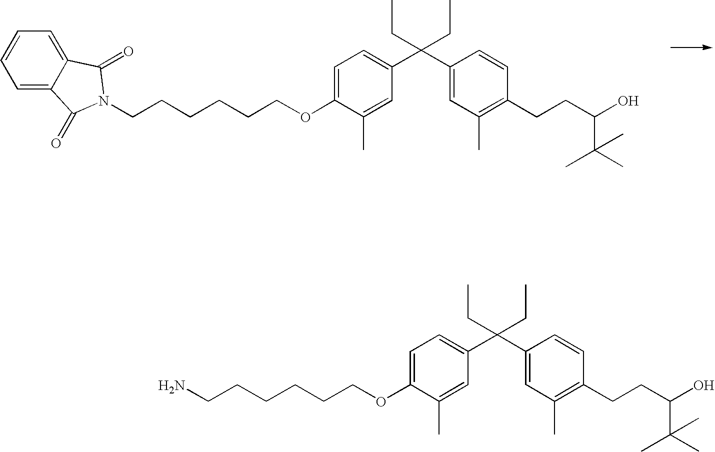

Using the same procedure as described for the preparation of Example 100-(2), the title compound was prepared from 2-[6-(4-{1-ethyl-1-[4-(3-hydroxy-4,4-dimethyl-pentyl)-3-methyl-phenyl]-propyl}-2-methyl-phenoxy)-hexyl]-isoindole-1,3-dione (compound prepared in Example 103-(1)). The yield was 60%.

¹H-NMR (300 MHz, CDCl₃): 0.59 (t, 6H), 0.89 (s, 9H), 1.35-1.56 (m, 7H), 1.74-1.84 (m, 3H), 2.03 (q, 4H), 2.15 (s, 3H), 2.25 (s, 3H), 2.50-2.60 (m, 1H), 2.70 (t, 2H), 2.81-2.91 (m, 1H), 3.24 (dd, 1H), 3.92 (t, 2H), 6.67 (d, 1H), 6.90-6.95 (m, 4H), 7.01 (d, 1H); MS (ESI+): 482 ([M+H]⁺).

### Example 104

### Preparation of 3-(4-{1-ethyl-1-[4-(3-ethyl-3-hydroxy-pent-1-ynyl)-3-methyl-phenyl]-propyl}-2-methyl-phenyl)-propionic acid

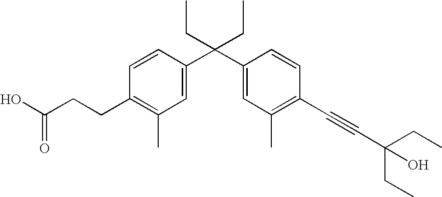

### (1) Preparation of 3-ethyl-1-trimethylsilanyl-pent-1-yn-3-ol

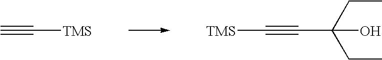

To a solution of TMS-acetylene (1.55g, 15.78mmol) in THF (20 ml) under nitrogen atmosphere at −40 degrees C. were added n-BuLi (7.5 mL, 18.93 mmol, 2.5M in hexane) and 3-pentanone (2.0 mL, 18.93 mmol) and the mixture was stirred for 3 h. The reaction mixture was diluted with diethyl ether, washed with sat. NH₄Cl aq., water and brine, dried over MgSO₄, filtered and concentrated under reduced pressure. The obtained residue was chromatographed on silica gel with EtOAc-n-Hexane (1:15) to give the title compound (2.8 g, 96%).

¹H NMR (CDCl₃): 1.86 (br, OH), 1.65 (m, 4H), 1.02 (t, 6H), 0.16 (s, 9H).

### (2) Preparation of 3-ethyl-pent-1-yn-3-ol

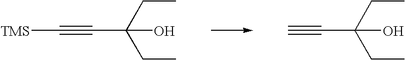

To a solution of 3-ethyl-1-trimethylsilanyl-pent-1-yn-3-ol (compound prepared in Example 104-(1)) (920 mg, 4.99 mmol) in diethyl ether (10 ml) under nitrogen atmosphere at 0 degrees C. was added TBAF (7.48 mL, 7.48 mmol, 1.0M in THF) and the mixture was stirred for 2 h. The reaction mixture was diluted with diethyl ether, and washed with sat. NH₄Cl aq., H₂O and brine, and dried over MgSO₄, filtered and concentrated under reduced pressure. The obtained residue was chromatographed on silica gel with EtOAc-n-Hexane (1:10) to give the title compound (200 mg, 35%).

¹H NMR (CDCl₃): 2.43 (s, 1H), 1.88 (br, OH), 1.69 (m, 4H), 1.04 (t, 6H).

### (3) Preparation of 3-{4-[1-ethyl-1-(3-methyl-4-trifluoromethanesulfonyloxy-phenyl)-propyl]-2-methyl-phenyl}-propionic acid methyl ester

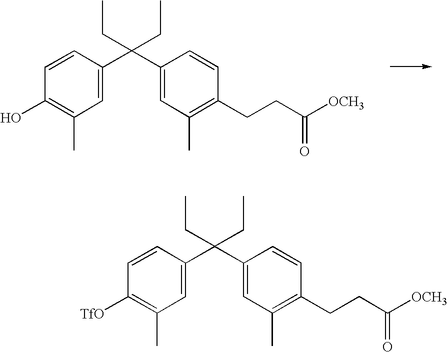

To a solution of 3-{4-[1-ethyl-1-(4-hydroxy-3-methyl-phenyl)-propyl]-2-methyl-phenyl}-propionic acid methyl ester (compound prepared in Example 18-(2)) (1.81 g, 5.1 mmol) in anhydrous CH₂Cl₂ (30 ml) under nitrogen atmosphere at room temperature were added trifluoromethanesulfonic anhydride (1.3 ml, 7.65 mmol) and pyridine (1.3 ml, 15.3 mmol). After stirring 1.5 h at room temperature, the mixture was poured into sat. NH₄Cl aq. and the products were extracted with CH₂Cl₂. The extracts were washed with brine, dried over MgSO₄, filtered and concentrated under reduced pressure. The obtained residue was chromatographed on amino functionalized silica gel with EtOAc-n-Hexane (1:10) to give the title compound (2.0 g, 80%).

¹H NMR (CDCl₃): 7.11-6.99 (m, 4H), 6.88 (m, 2H), 3.67 (s, 3H), 2.90 (dt, 2H), 2.58 (dt, 2H), 2.31 (s, 3H), 2.26 (s, 3H), 2.04 (q, 4H), 0.59 (t, 6H).

### (4) Preparation of 3-(4-{1-ethyl-1-[4-(3-ethyl-3-hydroxy-pent-1-ynyl)-3-methyl-phenyl]-propyl}-2-methyl-phenyl)-propionic acid methyl ester

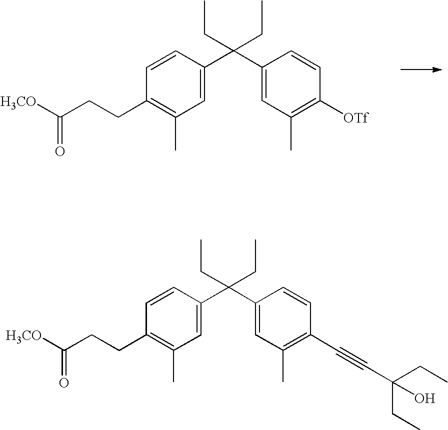

To a solution of 3-{4-[1-ethyl-1-(3-methyl-4-trifluoromethanesulfonyloxy-phenyl)-propyl]-2-methyl-phenyl}-propionic acid methyl ester (compound prepared in Example 104-(3)) (100 mg, 0.2 mmol) in anhydrous CH₃CN (2 m) under nitrogen atmosphere were added Pd(PPh₃)₄ (46 mg, 0.04 mmol), Cul (7.6 mg, 0.04 mmol), triethylamine (0.083 ml, 0.6 mmol) and 3-ethyl-pent-1-yn-3-ol (compound prepared in Example 104-(2)) (200 mg, 1.78 mmol). After stirring overnight at 100 degrees C., the mixture was poured into sat. NH₄Cl aq. and the products were extracted with diethyl ether. The extracts were washed with brine, dried over MgSO₄, filtered and concentrated under reduced pressure. The obtained residue was chromatographed on amino functionalized silica gel with EtOAc-n-Hexane (1:15) to give the title compound (40 mg, 44%).

¹H NMR (CDCl₃): 7.01-6.88 (m, 6H), 3.67 (s, 3H), 2.89 (dt, 2H), 2.57 (dt, 2H), 2.37 (s, 3H), 2.24 (s, 3H), 2.04 (q, 4H), 1.69 (q, 4H), 1.10 (t, 6H), 0.58 (t, 6H).

### (5) Preparation of 3-(4-{1-ethyl-1-[4-(3-ethyl-3-hydroxy-pent-1-ynyl)-3-methyl-phenyl]-propyl}-2-methyl-phenyl)-propionic acid

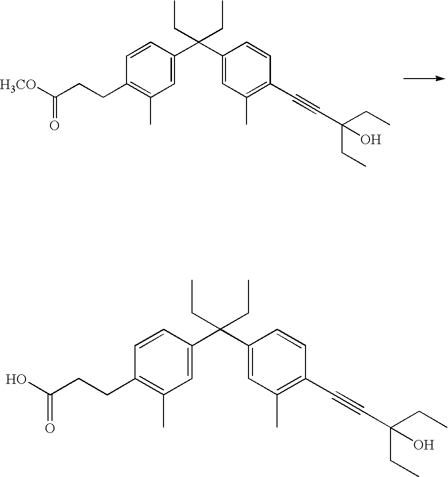

To a solution of 3-(4-{1-ethyl-1-[4-(3-ethyl-3-hydroxy-pent-1-ynyl)-3-methyl-phenyl]-propyl}-2-methyl-phenyl)-propionic acid methyl ester (compound prepared in Example 104-(4)) (40 mg, 0.089 mmol) in MeOH—H₂O (10:1, 1.1 ml) was added KOH as 0.5 tablets and the mixture was stirred at room temperature for 3 h. The reaction mixture was poured into sat. NH₄Cl aq. and the products were extracted with diethyl ether. The extracts were washed with water and brine, dried over MgSO₄, filtered and concentrated under reduced pressure. The obtained residue was chromatographed on amino functionalized silica gel with CH₂Cl₂/MeOH (20:1) to give the title compound (19 mg, 49%).

¹H NMR (CDCl₃): 7.37 (d, 1H), 6.98 (m, 2H), 6.90 (m, 3H), 2.89 (dt, 2H), 2.60 (dt, 2H), 2.36 (s, 3H), 2.22 (s, 3H), 2.04 (q, 4H), 1.75 (m, 4H), 1.10 (t, 6H), 0.58 (t, 6H); MS (ESI−): 433 ([M−H]⁼³¹ ).

### Example 105

### Preparation of 3-(4-{1-ethyl-1-[4-((E)-3-ethyl-3-hydroxy-pent-1-enyl)-3-methyl-phenyl]-propyl}-2-methyl-phenyl)-propionic acid

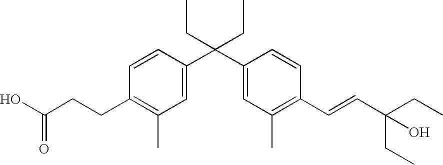

### (1) Preparation of 3-(4-{1-ethyl-1-[4-((E)-3-ethyl-3-hydroxy-pent-1-enyl)-3-methyl-phenyl]-propyl}-2-methyl-phenyl)-propionic acid methyl ester

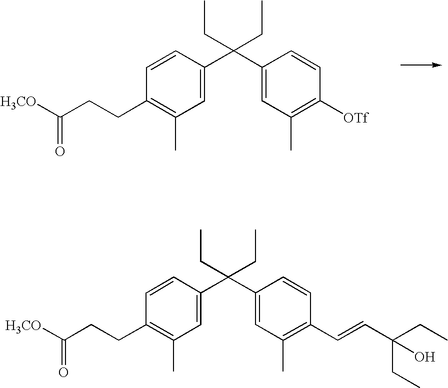

To a solution of 3-{4-[1-ethyl-1-(3-methyl4-trifluoromethanesulfonyloxy-phenyl)-propyl]-2-methyl-phenyl}-propionic acid methyl ester (compound prepared in Example 104-(3)) (170 mg, 0.35 mmol) in anhydrous DMF (4 ml) under nitrogen atmosphere were added triethylamine (0.058 ml, 0.42 mmol), 3-ethyl-pent-1-en-3-ol (1.0 g, 8.75 mmol), 1,3-bis(diphenylphosphine)propane (36 mg, 0.08 mmol) and palladium acetate (15.6 mg, 0.07 mmol). After stirring overnight at 100 degrees C., the reaction mixture was poured into water and the products were extracted with diethyl ether. The extracts were washed with water and brine, dried over Na₂SO₄, filtered and concentrated under reduced pressure. The obtained residue was chromatographed on amino functionalized silica gel with EtOAc-n-Hexane (1:10) to give the title compound (10 mg, 5%).

¹H NMR (CDCl₃); 6.95 (m, 6H), 6.74 (d, 1H), 6.00 (d, 1H), 3.67 (s, 3H), 2.89 (dt, 2H), 2.57 (dt, 2H), 2.30 (s, 3H), 2.25 (s, 3H), 2.04 (q, 4H), 1.649 (q, 4H), 0.91 (t, 6H), 0.60 (t, 6H).

### (2) Preparation of 3-(4-{1-ethyl-1-[4-((E)-3-ethyl-3-hydroxy-pent-1-enyl)-3-methyl-phenyl]-propyl}-2-methyl-phenyl)-propionic acid

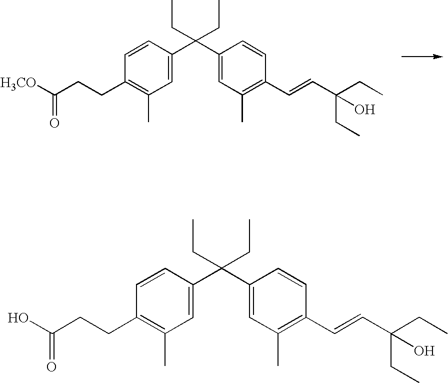

To a solution of 3-(4-{1-ethyl-1-[4-((E)-3-ethyl-3-hydroxy-pent-1-enyl)-3-methyl-phenyl]-propyl}-2-methyl-phenyl)-propionic acid methyl ester (compound prepared in Example 105-(1)) (10 mg, 0.022 mmol) in MeOH—H₂O (10:1, 1.1 ml) was added KOH as 0.5 tablets and the mixture was stirred at room temperature for 3 h. The reaction mixture was poured into sat. NH₄Cl aq. and the products were extracted with diethyl ether. The extracts were washed with water and brine, dried over MgSO₄, filtered and concentrated under reduced pressure. The obtained residue was chromatographed on amino functionalized silica gel with CH₂Cl₂/MeOH (20:1) to give the title compound (7.6 mg, 79%).

¹H NMR (CDCl₃): 6.95 (m, 6H), 6.74 (d, 1H), 6.00 (d, 1H), 2.90 (t, 2H), 2.62 (t, 2H), 2.30 (s, 3H), 2.25 (s, 3H), 2.04 (q, 4H), 1.63 (q, 4H), 0.91 (t, 6H), 0.60 (t, 6H); MS (ESI−): 435([M−H]⁻).

### Example 106

### Preparation of 3-(4-{1-ethyl-1-[3-methyl4-(4,4,4-trifluoro-3-hydroxy-3-trifluoromethyl-but-1-ynyl)-phenyl]-propyl}-2-methyl-phenyl)-propionic acid

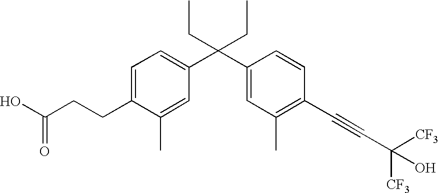

### (1) Preparation of Trifluoro-methanesulfonic acid 4-{1-ethyl-1-[3-methyl-4-(4,4,4-trifluoro-3-methoxymethoxy-3-trifluoromethyl-but-1-ynyl)-phenyl]-propyl}-2-methyl-phenyl ester

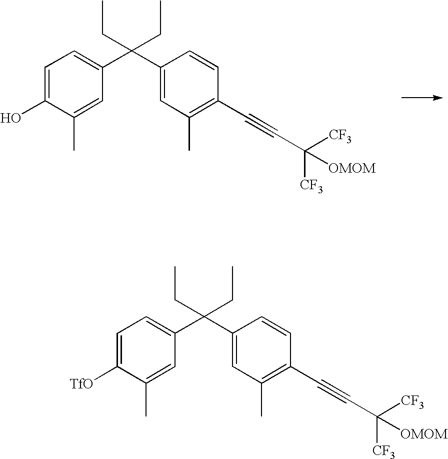

To a solution of 4-{1-ethyl-1-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) (400 mg, 0.8 mmol) in dichloromethane (4 ml), Tf₂O (0.15 ml, 0.89 mmol) and pyridine (0.13 ml, 1.6 mmol) were added at 0 degrees C. and the mixture was stirred at 0 degrees C. for 2 h. The reaction mixture was poured into water and the products were extracted with EtOAc. The extracts were washed with brine, dried over MgSO₄, filtered and concentrated under reduced pressure. The obtained residue was chromatographed on silica gel with EtOAc-n-Hexane (1:20) to give the title compound (410 mg, 81%)

¹H-NMR (CDCl₃): 0.60 (t, 6H, J=7.3 Hz), 2.07 (q, 4H, J=7.3 Hz), 2.32 (s, 3H), 2.41 (s, 3H), 3.48 (s, 3H), 5.15 (s, 2H), 6.90-7.05 (m, 4H), 7.11 (d, 1H, J=9.2 Hz), 7.40 (d, 1H, J=8.1 Hz).

### (2) Preparation of (E)-3-(4-{1-ethyl-1-[3-methyl-4-(4,4,4-trifluoro-3-methoxymethoxy-3-trifluoromethyl-but-1-ynyl)-phenyl]-propyl}-2-methyl-phenyl)-acrylic acid methyl ester

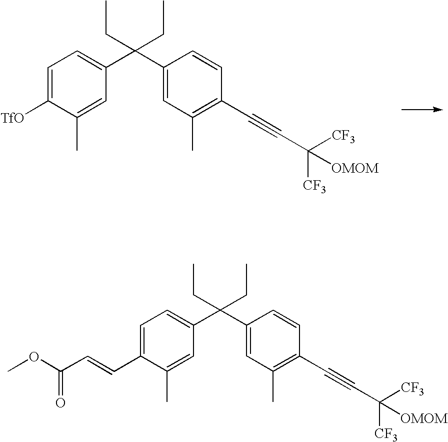

To a solution of Trifluoro-methanesulfonic acid 4-{1-ethyl-1-[3-methyl-4-(4,4,4-trifluoro-3-methoxymethoxy-3-trifluoromethyl-but-1-ynyl)-phenyl]-propyl}-2-methyl-phenyl ester (compound prepared in Example 106-(1)) (136 mg, 0.214 mmol) in DMF (2 ml) were added Pd(OAc)₂ (10 mg, 0.0429 mmol), dppp (22 mg, 0.0535 mmol), Et₃N (0.036 ml, 0.214 mmol) and ethylacrylae (0.029 ml, 0.321 mmol) and the mixture was stirred at 110 degrees C. for 5 h. The reaction mixture was cooled to room temperature, poured into water and the products were extracted with EtOAc. The extracts were washed with brine, dried over Na₂SO₄, filtered and concentrated under reduced pressure. The obtained residue was chromatographed on silica gel with EtOAc-n-Hexane (1:15) to give the title compound (53 mg, 43%)

¹H-NMR (300 MHz, CDCl₃): 0.60 (t, 6H), 2.07 (q, 4H), 2.37 (s, 3H), 2.38 (s, 3H), 3.46 (s, 3H), 3.79 (s, 3H), 5.13 (s, 2H), 6.31 (d, 1H), 6.94-7.03 (m, 4H), 7.36-7.44 (m, 2H), 7.92 (d, 1H).

### (3) Preparation of 3-(4-{1-ethyl-1-[3-methyl-4-(4,4,4-trifluoro-3-methoxymethoxy-3-trifluoromethyl-but-1-ynyl)-phenyl]-propyl}-2-methyl-phenyl)-propionic acid methyl ester

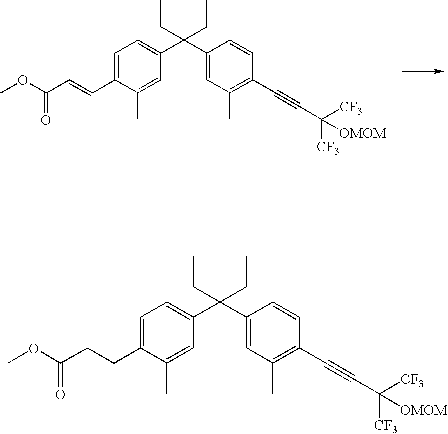

To a solution of (E)-3-(4-(1-ethyl-1-[3-methyl-4-(4,4,4-trifluoro-3-methoxymethoxy-3-trifluoromethyl-but-1-ynyl)-phenyl]-propyl}-2-methyl-phenyl)-acrylic acid methyl ester (compound prepared in Example 106-(2)) (36 mg, 0.0631 mmol) in CH₂Cl₂ (2 ml) and MeOH (4 ml) at 0 degrees C. were added NiCl₂-6H₂O (30 mg, 0.126 mmol), and NaBH₄ (51 mg, 1.33 mmol) and the mixture was stirred at room temperature for 30 min. The reaction mixture was poured into sat. NH₄Cl aq. and the products were extracted with EtOAc. The extracts were washed with brine, dried over Na₂SO₄, filtered and concentrated under reduced pressure. The obtained residue was chromatographed on silica gel with EtOAc-n-Hexane (1:25) to give the title compound (30 mg, 84%).

¹H-NMR (300 MHz, CDCl₃): 0.52 (t, 6H), 1.99 (q, 4H), 2.17 (s, 3H), 2.32 (s, 3H), 2.50 (t, 2H), 2.82 (t, 2H), 3.40 (s, 3H), 3.60 (s, 3H), 5.13 (s, 2H), 6.79-6.98 (m, 5H), 7.29 (d, 1H); MS (ESI+): 573 ([M+H]⁺).

### (4) Preparation of 3-(4-{1-ethyl-1-[3-methyl-4-(4,4,4-trifluoro-3-hydroxy-3-trifluoromethyl-but-1-ynyl)-phenyl]-propyl}-2-methyl-phenyl)-propionic acid methyl ester

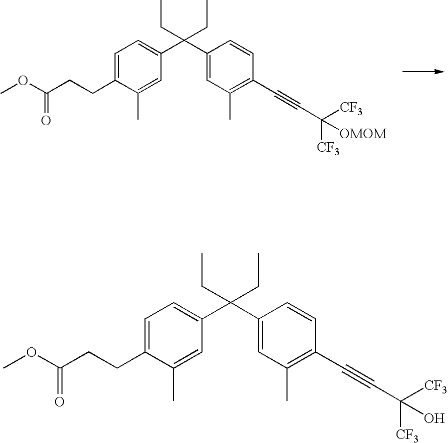

To a solution of 3-(4-{1-ethyl-1-[3-methyl-4-(4,4,4-trifluoro-3-methoxymethoxy-3-trifluoromethyl-but-1-ynyl)-phenyl]-propyl}-2-methyl-phenyl)-propionic acid methyl ester (compound prepared in Example 106-(3)) (35 mg, 0.0611 mmol) in i-PrOH (0.8 ml) were added CBr₄ (2 mg, 0.0061 mmol) and the mixture was refluxed overnight. The reaction mixture was cooled to room temperature and concentrated under reduced pressure. The obtained residue was chromatographed on silica gel with EtOAc-n-Hexane (1:8) to give the title compound (28 mg, 86%).

¹H-NMR (300 MHz, CDCl₃): 0.58 (t, 6H), 2.05 (q, 4H), 2.24 (s, 3H), 2.38 (s, 3H), 2.52 (t, 2H), 2.89 (m, 2H), 3.67 (s, 3H), 6.85-7.05 (m, 5H), 7.34 (d, 1H).

### (5) Preparation of 3-(4-{1-ethyl-1-[3-methyl-4-(4,4,4-trifluoro-3-hydroxy-3-trifluoromethyl-but-1-ynyl)-phenyl]-propyl}-2-methyl-phenyl)-propionic acid

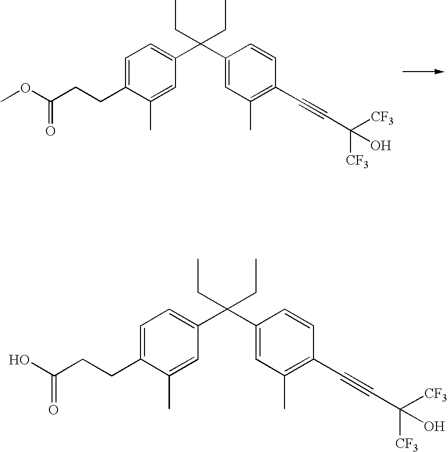

To a solution of 3-(4-{1-ethyl-1-[3-methyl-4-(4,4,4-trifluoro-3-hydroxy-3-trifluoromethyl-but-1-ynyl)-phenyl]-propyl}-2-methyl-phenyl)-propionic acid methyl ester (compound prepared in Example 106-(4)) (30 mg, 0.0539 mmol) in MeOH (1 ml) was added 1N—KOH(0.2 ml, 0.2 mmol) and the mixture was stirred at room temperature overnight. The reaction mixture was poured into sat. NH₄Cl aq. and the products were extracted with EtOAc. The extracts were washed with brine, dried over Na₂SO₄, filtered and concentrated under reduced pressure. The obtained residue was chromatographed on silica gel with CH₂Cl₂-MeOH (20:1) to give the title compound (22 mg, 75%).

¹H-NMR (300 MHz, CDCl₃): 0.59 (t, 6H), 2.05 (q, 4H), 2.24 (s, 3H), 2.38 (s, 3H), 2.61 (t, 2H), 2.90 (t, 2H), 3.67 (s, 3H), 6.87-7.05 (m, 5H), 7.34 (d, 1H); MS (ESI−): 513 ([M−H]⁻).

### Example 107

### Preparation of 3-(4-{1-ethyl-1-[3-methyl-4-((E)-4,4,4-trifluoro-3-hydroxy-3-trifluoromethyl-but-1-enyl)-phenyl]-propyl}-2-methyl-phenyl)-propionic acid

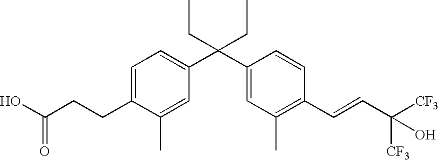

### (1) Preparation of trifluoro-methanesulfonic acid 4-{1-ethyl-1-[3-methyl-4-((E)-4,4,4-trifluoro-3-methoxymethoxy-3-trifluoromethyl-but-1-enyl)-phenyl]-propyl}-2-methyl-phenyl ester

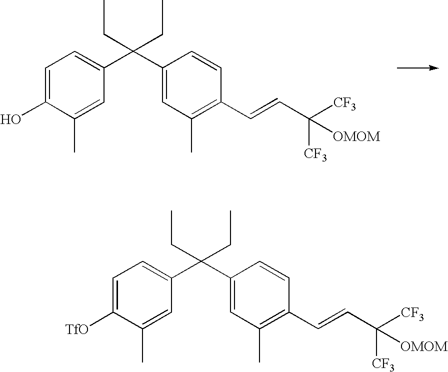

To a stirred solution of 4-{1-ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in Example 30-(1)) (394 mg, 0.78 mmol) in 7.80 ml of CH₂Cl₂ were added Tf₂O (0.197 ml, 1.17 mmol) and Et₃N at −30 degrees C. under N₂ atmosphere. After stirred for 1 h, the reaction mixture was quenched with 30 ml of sat. NaHCO₃ aq. and the products were extracted with CH₂Cl₂ The extracts were washed with brine, dried over anhydrous Na₂SO₄, filtered and evaporated under reduced pressure. The obtained residue was purified by silica gel chromatography (n-Hexane:EtOAc=5:1) to give the title compound (440 mg, 88.7%) as colorless oil.

¹H-NMR (300 MHz, CDCl₃): 7.40-7.30 (m, 2H), 7.15-6.95 (m, 5H), 6.10 (d, 1H), 5.00 (s, 2H), 3.50 (s, 3H), 2.35 (s, 6H), 2.10 (q, 4H), 0.60 (t, 6H).

### (2) Preparation of (E)-3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-acrylic acid ethyl ester

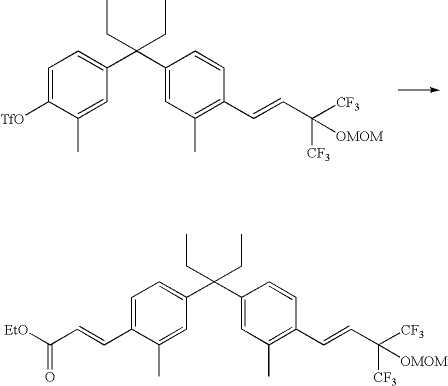

To a stirred solution of trifluoro-methanesulfonic acid 4-{1-ethyl-1-[3-methyl-4-((E)-4,4,4-trifluoro-3-methoxymethoxy-3-trifluoromethyl-but-1-enyl)-phenyl]-propyl}-2-methyl-phenyl ester (compound prepared in Example 107-(1)) (234 mg, 0.37 mmol) in DMF (3.7 ml) were added Pd(OAc)₂ (16.6 mg, 20 mol %), dppp (38.2 mg, 25 mol %), ethylacrylate (0.40 ml, 3.70 mmol) and Et₃N (0.103 ml, 0.74 mmol) at room temperature under N₂ atmosphere. Then the reaction mixture was stirred at 110 degrees C. for 16 h. The reaction mixture was cooled to room temperature, poured into water and the products were extracted with EtOAc. The extracts were dried over anhydrous Na₂SO₄, filtered and evaporated under reduced pressure. The obtained residue was purified by silica gel chromatography (n-Hexane:EtOAc=10:1) to give the title compound (138 mg, 63.6%) as colorless sticky oil.

¹H-NMR(300 MHz, CDCl₃): 7.95 (d, 1H), 7.50-7.35 (m, 3H), 7.05-6.95 (m, 4H), 6.35 (d, 1H), 6.08 (d, 1H), 5.00 (s, 2H), 4.25 (q, 2H), 3.50 (s, 3H), 2.40 (s, 3H), 2.35 (s, 3H), 2.10 (q, 4H), 1.35 (t, 3H), 0.60 (t, 6H).

### (3) Preparation of 3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-propionic acid ethyl ester

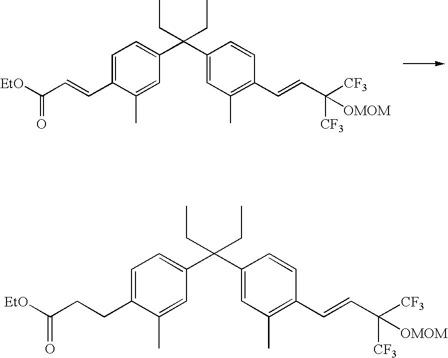

To a stirred solution of (E)-3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-acrylic acid ethyl ester (compound prepared in Example 107-(2)) (138 mg, 0.24 mmol) in MeOH/CH₂Cl₂ (2/1 v/v, 30 ml) was added NiCl₂ 6H₂O (114 mg, 0.48 mmol) and NaBH₄ at 0 degrees C. under N₂ atmosphere and the mixture was stirred for 1 h. The reaction mixture was poured into sat. NH₄Cl aq. and the products were extracted with EtOAc The extracts were dried over anhydrous Na₂SO₄, filtered and evaporated under reduced pressure. The obtained residue was purified by silica gel chromatography (n-Hexane:EtOAc=10:1) to give the title compound (116 mg, 82.3%) as colorless sticky oil.

¹H-NMR (300 MHz, CDCl₃): 7.40-7.30 (m, 2H), 7.05-6.85 (m, 5H), 6.08 (d, 1H), 5.00 (s, 2H), 4.15 (q, 2H), 3.50 (s, 3H), 2.90 (t, 2H), 2.55 (t, 2H), 2.35 (s, 3H), 2.30 (s, 3H), 2.10 (q, 4H), 1.35 (t, 3H), 0.60 (t, 6H).

### (4) Preparation of Preparation of 3-(4-{1-ethyl-1-[3-methyl-4-((E)4,4,4-trifluoro-3-hydroxy-3-trifluoromethyl-but-1-enyl)-phenyl]-propyl}-2-methyl-phenyl)-propionic acid

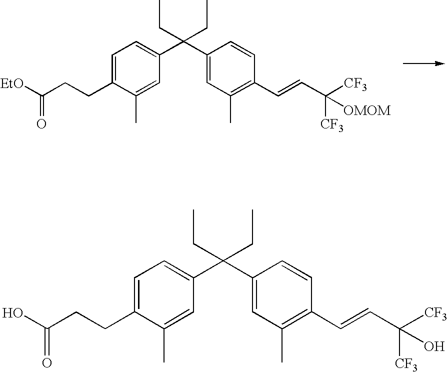

To a stirred solution of 3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-propionic acid ethyl ester (compound prepared in Example 107-(3)) (116 mg, 0.20 mmol) in EtOH (2 ml) was added CBr₄ (6.60 mg, 10 mol %) under N₂ atmosphere at room temperature. The reaction mixture was refluxed for 4.5 h. The reaction mixture was poured into water and the products were extracted with EtOAc. The extracts were washed with water and brine, dried over Na₂SO₄, filtered and concentrated under reduced pressure. The obtained residue was chromatographed on silica gel with EtOAc-n-Hexane (1:10) to give 3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-propionic acid ethyl ester (84.0 mg, 78.5%) as colorless sticky oil.

To a stirred solution of 3-(4-{1-ethyl-1-[3-methyl-4-((E)-4,4,4-trifluoro-3-methoxymethoxy-3-trifluoromethyl-but-1-enyl)-phenyl]-propyl}-2-methyl-phenyl)-propionic acid ethyl ester (84.0 mg, 0.15 mmol) in EtOH/Water (4/1 v/v, 1.9 ml) was added KOH (16.8 mg, 0.30 mmol) at room temperature and the reaction mixture was stirred for4 h. The reaction mixture was poured into 0.1N—HCl and the products were extracted with EtOAc. The extracts were washed with water and brine, dried over anhydrous Na₂SO₄, filtered and evaporated under reduced pressure. The obtained residue was purified by silica gel chromatography (n-Hexane:EtOAc=1:1) to give the title compound (69.0 mg, 89.0%) as colorless sticky oil.

¹H-NMR (300 MHz, CDCl₃): 7.40-7.30 (m, 2H), 7.05-6.85 (m, 5H), 6.10 (d, 1H), 2.90 (t, 2H), 2.65 (t, 2H), 2.35 (s, 3H), 2.25 (s, 3H), 2.15-2.00 (m, 5H), 0.60 (t, 6H); MS (ESI−): 515 ([M−H]⁻).

### Example 108

### Preparation of 3-(4-{1-ethyl-1-[4-(3-hydroxy-4,4-dimethyl-pentyl)-3-methyl-phenyl]-propyl}-2-methyl-phenyl)-propionic acid

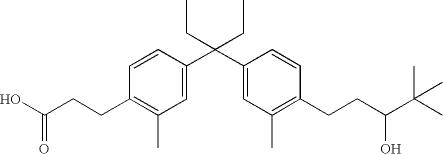

### (1) Preparation of trifluoro-methanesulfonic acid 4-{1-[4-(4,4-dimethyl-3-oxo-pentyl)-3-methyl-phenyl]-1-ethyl-propyl}-2-methyl-phenyl ester

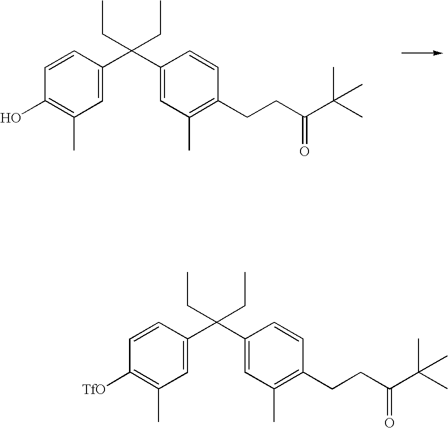

To a solution of 1-{4-[1-ethyl-1-(4-hydroxy-3-methyl-phenyl)-propyl]-2-methyl-phenyl}-4,4-dimethyl-pentan-3-one (compound prepared in Example 18-(3)) (410 mg, 1.07 mmol) in anhydrous CH₂Cl₂ (10 ml) under nitrogen atmosphere were added trifluoromethanesulfonic anhydride (0.26 ml, 1.6 mmol) and pyridine (0.26 ml, 3.21 mmol) and the mixture was stirred at room temperature for 1 h. The reaction mixture was poured into sat. NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:7) to give the title compound (470 mg, 85%).

¹H NMR (CDCl₃): 7.10-6.97 (m, 4H), 6.88 (m, 2H), 2.82 (m, 2H), 2.72 (m, 2H), 2.31 (s, 3H), 2.25 (s, 3H), 2.04 (q, 4H), 1.10 (s, 9H), 0.59 (t, 6H).

### (2) Preparation of (E)-3-(4-{1-[4-(4,4-dimethyl-3-oxo-pentyl)-3-methyl-phenyl]-1-ethyl-propyl}-2-methyl-phenyl)-acrylic acid methyl ester

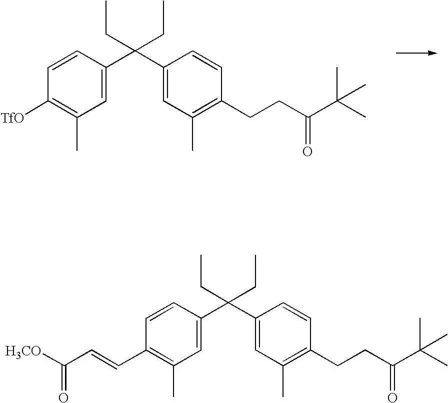

To a solution of trifluoro-methanesulfonic acid 4-{1-[4-(4,4-dimethyl-3-oxo-pentyl)-3-methyl-phenyl]-1-ethyl-propyl}-2-methyl-phenyl ester (compound prepared in Example 108-(1)) (470 mg, 0.91 mmol) in anhydrous DMF (10 ml) under nitrogen atmosphere were added triethylamine (0.15 ml, 1.08 mmol), methylacrylate (0.12 ml, 1.3 mmol), 1,3-bis(diphenylphosphine)propane (93 mg, 0.22 mmol) and palladium acetate (40 mg, 0.18 mmol). The reaction mixture was stirred at 100 degrees C. for 1.5 h and poured into sat. NH₄Cl aq. and the products were extracted with ethyl acetate. The extracts were washed with brine, dried over MgSO₄, filtered and concentrated under reduced pressure. The obtained residue was chromatographed on silica gel with EtOAc-n-Hexane (1:7) to give the title compound (230 mg, 56%).

¹H NMR (CDCl₃): 7.95 (d, 1H), 7.42 (d, 1H), 7.04-6.96 (m, 3H), 6.90 (m, 2H), 6.32 (d, 1H), 3.79 (s, 3H), 2.82 (m, 2H), 2.71 (m, 2H), 2.38 (s, 3H), 2.24 (s, 3H), 2.06 (q, 4H), 1.09 (s, 9H), 0.60 (t, 6H).

### (3) Preparation of (E)-3-(4-{1-ethyl-1-[4-(3-hydroxy-4,4-dimethyl-pentyl)-3-methyl-phenyl]-propyl}-2-methyl-phenyl)-acrylic acid methyl ester

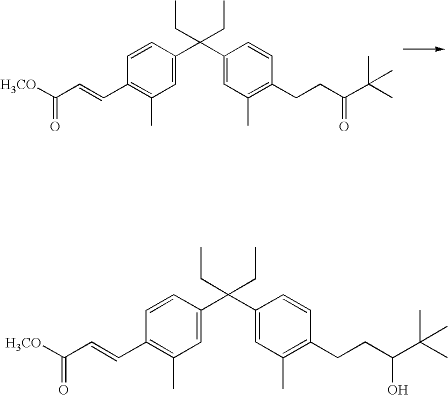

To a solution of (E)-3-(4-{1-[4-(4,4-dimethyl-3-oxo-pentyl)-3-methyl-phenyl]-1-ethyl-propyl}-2-methyl-phenyl)-acrylic acid methyl ester (compound prepared in Example 108-(2)) (220 mg, 0.49 mmol) in methanol (5 ml) under nitrogen atmosphere was added sodium borohydride (37 mg, 0.98 mmol) and the mixture was stirred at room temperature for 1.5 h. The reaction mixture was poured into water and the products were extracted with ethyl acetate. The extracts were washed with brine, dried over MgSO₄, filtered and concentrated under reduced pressure. The obtained residue was chromatographed on silica gel with EtOAc-n-Hexane (1:7) to give the title compound (160 mg, 72%).

¹H NMR (CDCl₃): 7.95 (d, 1H), 7.43 (d, 1H), 7.04-6.98 (m, 3H), 6.91 (m, 2H), 6.31 (d, 1H), 3.79 (s, 3H), 3.24 (m, 1H), 2.86 (m, 1H), 2.56 (m, 1H), 2.39 (s, 3H), 2.25 (s, 3H), 2.08 (q, 4H), 1.79 (m, 1H), 1.48 (m, 1H), 0.89 (s, 9H), 0.61 (t, 6H).

### (4) Preparation of 3-(4-{1-ethyl-1-[4-(3-hydroxy-4,4-dimethyl-pentyl)-3-methyl-phenyl]-propyl}-2-methyl-phenyl)-propionic acid

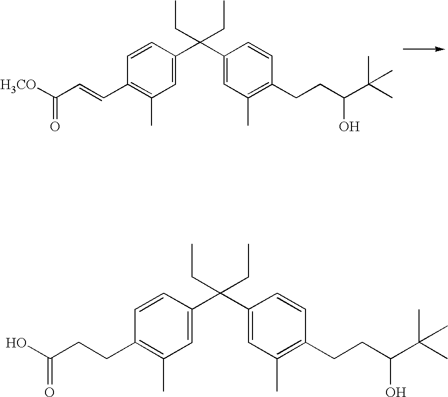

To a solution of (E)-3-(4-{1-ethyl-1-[4-(3-hydroxy-4,4-dimethyl-pentyl)-3-methyl-phenyl]-propyl}-2-methyl-phenyl)-acrylic acid methyl ester (compound prepared in Example 108-(3)) (160 mg, 0.35 mmol) in methanol (3 ml) was added 10% palladium carbon (10 mg) and the mixture was stirred overnight at room temperature under hydrogen atmosphere. The reaction mixture was filtered through celite and the filtrate was concentrated under reduced pressure. The obtained residue was dissolved in methanol (3 ml)/H₂O (0.1 ml). To the mixture was added KOH as 1 tablet and the mixture was stirred at room temperature for 30 min. The reaction mixture was poured into 0.1 N—HCl and the products were extracted with ethyl acetate. The extracts were washed with brine, dried over MgSO₄, filtered and concentrated under reduced pressure. The obtained residue was chromatographed on silica gel with EtOAc-n-Hexane (1:3) to give the title compound (110 mg, 71% for two steps) as a solid.

¹H NMR (CDCl₃): 7.03-6.89 (m, 6H), 3.25 (dt, 1H), 2.86 (m,. 3H), 2.58 (m, 3H), 2.25 (s, 6H), 2.04 (q, 4H), 1.79 (m, 1H), 1.50 (m, 1H), 0.88 (s, 9H), 0.59 (t, 6H), MS (ESI−) : 437 ([M−H]⁻).

### Example 109

### Preparation of (E)-3-ethyl-1-[4-(1-ethyl-1-{3-methyl-4-[4-(1H-tetrazol-5yl)-butoxy]-phenyl}-propyl)-2-methyl-phenyl]-pent-1-en-3-ol

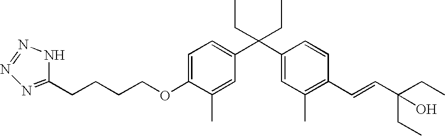

### (1) Preparation of 5-(4-{1-ethyl-1-[4-((E)-3-ethyl-3-hydroxy-pent-1-enyl)-3-methyl-phenyl]-propyl}-2-methyl-phenoxy)-pentanenitrile

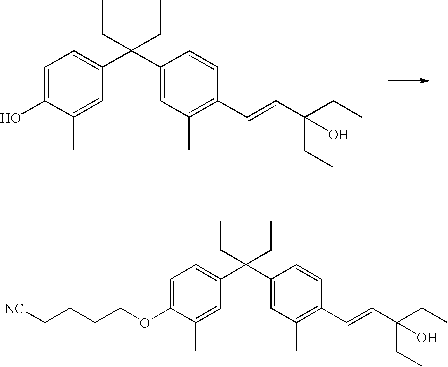

To a stirred solution of 4-{1-ethyl-1-[4-((E)-3-ethyl-3-hydroxy-pent-1-enyl)-3-methyl-phenyl]-propyl}-2-methyl-phenol (compound prepared in Example 1-(5)) (274 mg, 0.72 mmol) and 5-Bromovaleronitrile (350 mg, 2.16 mmol) in 7.20 ml of anhydrous DMF was added K₂CO₃ (299 mg, 2.16 mmol) under N₂ atmosphere at room temperature. The reaction mixture was stirred at 100 degrees C. for 12 h. The reaction mixture was cooled to room temperature, poured into water and the products were extracted with EtOAc. The extracts were washed with brine, dried over Na₂SO₄, filtered and concentrated under reduced pressure. The obtained residue was chromatographed on silica gel with EtOAc-n-Hexane (1:6) to give the title compound (184 mg, 55%) as colorless oil.

¹H-NMR (300 MHz, CDCl₃): 7.30 (d, 1H), 7.00-6.85 (m, 4H), 6.75 (d, 1H), 6.65 (d, 1H), 6.00 (d, 1H), 4.00 (t, 2H), 2.50 (t, 2H), 2.35 (s, 3H), 2.25 (s, 3H), 2.15 (s, 3H), 2.10-1.85 (m, 8H), 1.70-1.55 (m, 4H), 0.90 (t, 6H), 0.60 (t, 6H).

### (2) Preparation of (E)-3-ethyl-1-[4-(1-ethyl-1-{3-methyl-4-[4-(1H-tetrazol-5-yl)-butoxy]-phenyl}-propyl)-2-methyl-phenyl]-pent-1-en-3-ol

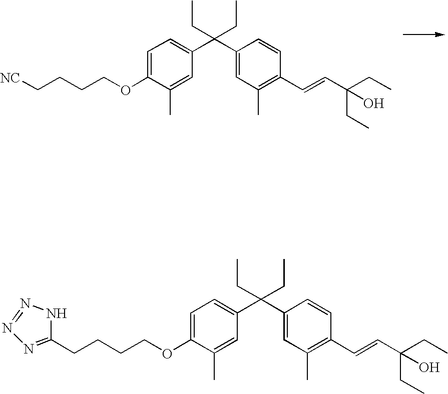

To a solution of 5-(4-{1-ethyl-1-[4-((E)-3-ethyl-3-hydroxy-pent-1-enyl)-3-methyl-phenyl]-propyl}-2-methyl-phenoxy)-pentanenitrile (compound prepared in Example 109-(1)) (46.5 mg, 0.10 mmol) in anhydrous toluene (1.2 ml) was added Me₃SnN₃ (61.7 mg, 0.30 mmol) and the mixture was stirred at 110 degrees C. for 12 h. The reaction mixture was cooled to room temperature, poured into water and the products were extracted with EtOAc. The extracts were washed with water and brine, dried over Na₂SO₄, filtered and concentrated under reduced pressure. The obtained residue was chromatographed on silica gel with CH₂Cl₂: MeOH (50:1) to give the title compound (13 mg, 26%) as amorphous.

¹H-NMR(300 MHz, CDCl₃): 7.30 (d, 1H), 7.00-6.85 (m, 4H), 6.75 (d, 1H), 6.65 (d, 1H), 6.00 (d, 1H), 3.98 (t, 2H), 3.10 (t, 2H), 2.35 (s, 3H), 2.25 (s, 3H), 2.15 (s, 3H), 2.10-1.95 (m, 8H), 1.90-1.80 (m, 2H), 1.70-1.55 (m, 4H), 0.90 (t, 6H), 0.60 (t, 6H); MS (ESI−): 503 ([M−H]⁺).

### Example 110

### Preparation of 1-[4-(1-ethyl-1-{3-methyl-4-[4-(1 H-tetrazol-5-yl)-butoxy]-phenyl}-propyl)-2-methyl-phenylethynyl]-cyclopentanol

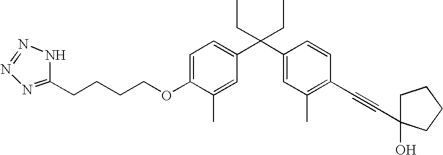

### (1)Preparation of 5-(4-{1-ethyl-1-[4-(1-hydroxy-cyclopentylethynyl)-3-methyl-phenyl]-propyl}-2-methyl-phenoxy)-pentanenitrile

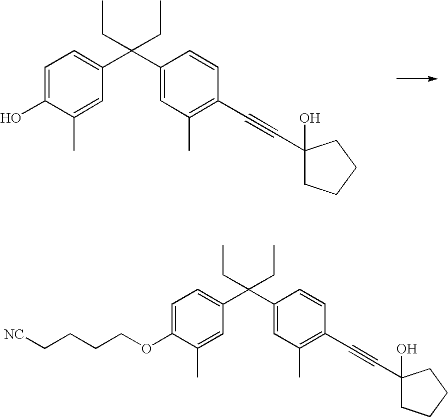

Using the same procedure as described for the preparation of Example 109-(1), the title compound was prepared from 4-{1-Ethyl-1-[4-(1-hydroxy-cyclopentylethynyl)-3-methyl-phenyl]-propyl}-2-methyl-phenol (compound prepared in Example 7). The yield was 55%.

¹H-NMR (300 MHz, CDCl₃): 7.30 (d, 1H), 7.00 (bs, 1H), 6.95-6.85 (m, 2H), 6.85 (bs, 1H), 6.65 (d, 1H), 3.98 (t, 2H), 2.50 (t, 2H), 2.38 (s, 3H), 2.15 (s, 3H), 2.10-1.70 (m, 17H), 0.60 (t, 6H).

### (2) Preparation of 1-[4-(1-ethyl-1-{3-methyl4-[4-(1H-tetrazol-5-yl)-butoxy]-phenyl}-propyl)-2-methyl-phenylethynyl]-cyclopentanol

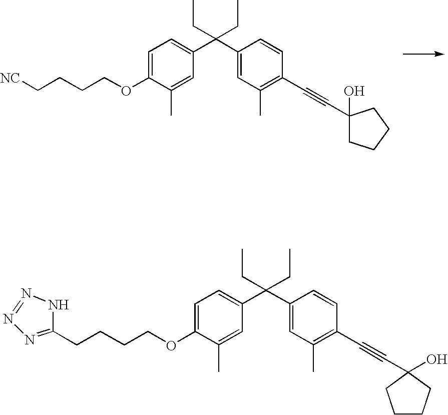

Using the same procedure as described for the preparation of Example 109-(2), the title compound was prepared from 5-(4-{1-Ethyl-1-[4-(1-hydroxy-cyclopentylethynyl)-3-methyl-phenyl]-propyl}-2-methyl-phenoxy)-pentanenitrile (compound prepared in Example 110-(1)). The yield was 44%.

¹H-NMR (300 MHz, CDCl₃): 7.25 (d, 1H), 7.00 (bs, 1H), 6.95-6.85 (m, 2H), 6.85 (bs, 1H), 6.65 (d, 1H), 3.98 (t, 2H), 3.15 (t, 2H), 2.35 (s, 3H), 2.15 (s, 3H), 2.10-1.70 (m, 17H), 0.60 (t, 6H); MS (ESI−) :499 ([M−H]⁻).

### Example 111

### Preparation of 1-{(E)-2-[4-(1-ethyl-1-{3-methyl-4-[4-(1H-tetrazol-5-yl)-butoxy]-phenyl}-propyl)-2-methyl-phenyl]-vinyl}-cyclopentanol

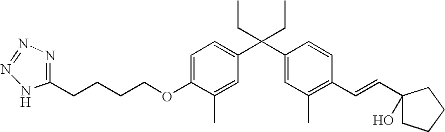

### (1) Preparation of 5-[4-(1-ethyl-1-{4-[(E)-2-(1-hydroxy-cyclopentyl)-vinyl]-3-methyl-phenyl}-propyl)-2-methyl-phenoxy]-pentanenitrile

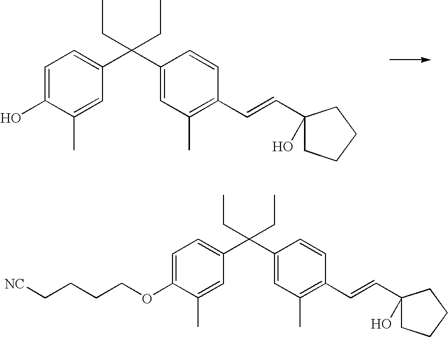

Using the same procedure as described for the preparation of Example 109-(1), the title compound was prepared from 4-(1-Ethyl-1-{4-[(E)-2-(1-hydroxy-cyclopentyl)-vinyl]-3-methyl-phenyl}-propyl)-2-methyl-phenol (compound prepared in Example 10). The yield was 96%.

¹H-NMR (300 MHz, CDCl₃): 0.60 (t, 6H), 1.69-1.97 (m, 8H), 2.00-2.07 (m, 6H), 2.14 (s, 3H), 2.30 (s, 3H), 2.46 (t, 2H), 3.97 (t, 2H), 6.25 (d, 1H), 6.66 (d, 1H), 6.83 (d, 1H), 6.90-6.95 (m, 4H), 7.32 (d, 1H).

### (2) Preparation of 1-{(E)-2-[4-(1-ethyl-1-{3-methyl-4-[4-(1H-tetrazol-5-yl)-butoxy]-phenyl}-propyl)-2-methyl-phenyl]-vinyl}-cyclopentanol

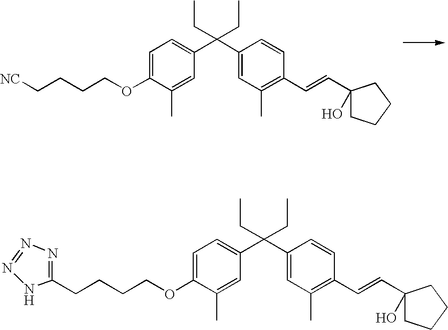

Using the same procedure as described for the preparation of Example 109-(2), the title compound was prepared from 5-[4-(1-ethyl-1-{4-[(E)-2-(1-hydroxy-cyclopentyl)-vinyl]-3-methyl-phenyl}-propyl)-2-methyl-phenoxy]-pentanenitrile (compound prepared in Example 111-(1)). The yield was 4%.

¹H-NMR (300 MHz, CDCl₃): 0.60 (t, 6H), 1.69-1.95 (m, 8H), 2.00-2.09 (m, 6H), 2.15 (s, 3H), 2.30 (s, 3H), 3.10 (t, 2H), 4.00 (t, 2H), 6.25 (d, 1H), 6.68 (d, 1H), 6.83 (d, 1H), 6.90-6.95 (m, 4H), 7.32 (d, 1H); MS (ESI−): 501 ([M−H]⁻).

### Example 112

### Preparation of 1-[4-(1-ethyl-1-{3-methyl4-[4-(1 H-tetrazol-5-yl)-butoxy]-phenyl}-propyl)-2-methyl-phenylethynyl]-cyclohexanol

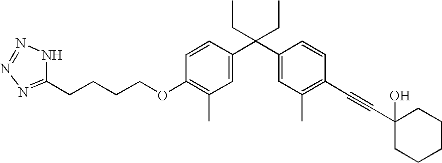

### (1) Preparation of 5-(4-{1-Ethyl-1-[4-(1-hydroxy-cyclohexylethynyl)-3-methyl-phenyl]-propyl}-2-methyl-phenoxy)-pentanenitrile

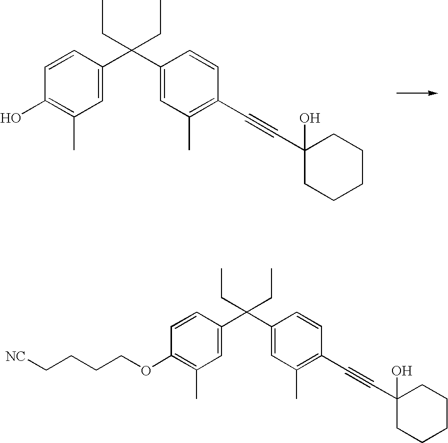

Using the same procedure as described for the preparation of Example 109-(1), the title compound was prepared from 4-{1-ethyl-1-[4-(1-hydroxy-cyclohexylethynyl)-3-methyl-phenyl]-propyl)-2-methyl-phenol (compound prepared in Example 8). The yield was 70%.

¹H-NMR (300 MHz, CDCl₃): 0.59 (t, 6H), 1.57-2.00 (m, 14H), 2.03 (q, 4H), 2.14 (s, 3H), 2.38 (s, 3H), 2.50 (t, 2H), 3.98 (t, 2H), 6.66 (d, 1H), 6.85-7.00 (m, 4H), 7.27 (d, 1H).

### (2) Preparation of 1-[4-(1-ethyl-1-{3-methyl4-[4-(1H-tetrazol-5-yl)-butoxy]-phenyl}-propyl)-2-methyl-phenylethynyl]-cyclohexanol

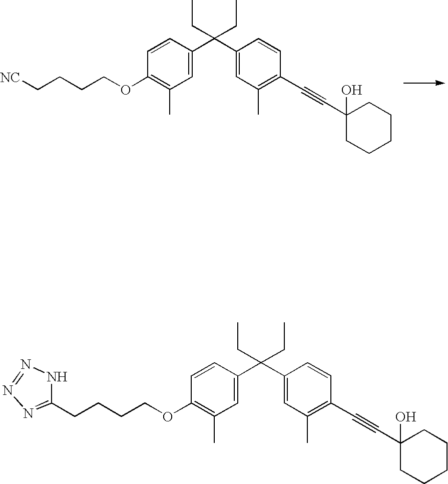

Using the same procedure as described for the preparation of Example 109-(2), the title compound was prepared from 5-(4-{1-Ethyl-1-[4-(1-hydroxy-cyclohexylethynyl)-3-methyl-phenyl]-propyl}-2-methyl-phenoxy)-pentanenitrile (compound prepared in Example 112-(1)). The yield was 26%.

### ¹H-NMR (300 MHz, CDCl₃): 0.59 (t, 6H), 1.57-2.00 (m, 14H), 2.03 (q, 4H), 2.14 (s, 3H), 2.37 (s, 3H), 3.10 (t, 2H), 3.99 (t, 2H), 6.67 (d, 1H), 6.86-7.00 (m, 4H), 7.26 (d, 1H); MS (ESI+): 537 ([M+Na]⁺).

### Example 113

### Preparation of 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclohexanol

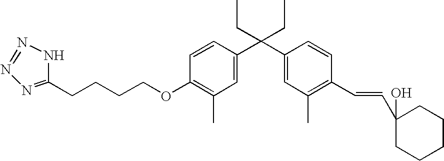

### (1) Preparation of 5-[4-(1-ethyl-1-{4-[(E)-2-(1-hydroxy-cyclohexyl)-vinyl]-3-methyl-phenyl}-propyl)-2-methyl-phenoxy]-pentanenitrile

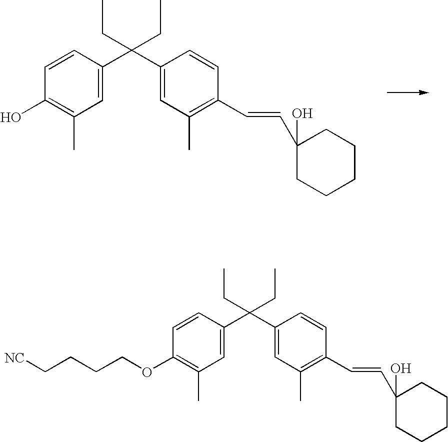

Using the same procedure as described for the preparation of Example 109-(1), the title compound was prepared from 4-(1-ethyl-1-{4-[(E)-2-(1-hydroxy-cyclohexyl)-vinyl]-3-methyl-phenyl}-propyl)-2-methyl-phenol (compound prepared in Example 11). The yield was 73%.

¹H-NMR (300 MHz, CDCl₃): 0.60 (t, 6H); 1.57-2.00 (m, 14H), 2.04 (q, 4H), 2.14 (s, 3H), 2.30 (s, 3H), 2.44 (m, 2H), 3.98 (t, 2H), 6.20 (d, 1H), 6.66 (d, 1H), 6.81 (d, 1H), 6.90-6.96 (m, 4H), 7.32 (d, 1H).

### (2) Preparation of 1-{(E)-2-[4-(1-ethyl-1-{3-methyl4-[4-(1H-tetrazol-5-yl)-butoxy]-phenyl}-propyl)-2-methyl-phenyl]-vinyl}-cyclohexanol

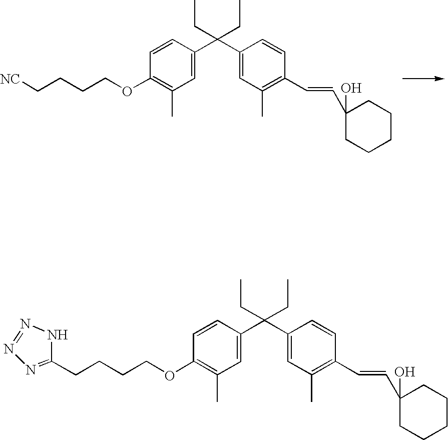

Using the same procedure as described for the preparation of Example 109-(2), the title compound was prepared from 5-[4-(1-Ethyl-1-{4-[(E)-2-(1-hydroxy-cyclohexyl)-vinyl]-3-methyl-phenyl}-propyl)-2-methyl-phenoxy]-pentanenitrile (compound prepared in Example 113-(1)). The yield was 24%.

¹H-NMR (300 MHz, CDCl₃): 0.60 (t, 6H), 1.57-2.00 (m, 14H), 2.04 (q, 4H), 2.14 (s, 3H), 2.29 (s, 3H), 3.10 (t, 2H), 3.98 (t, 2H), 6.20 (d, 1H), 6.67 (d, 1H), 6.81 (d,1 H), 6.90-6.96 (m, 4H), 7.31 (d,1 H); MS (ESI−): 515 ([M−H]⁻).

### Example 114

### Preparation of 4-[4-(1-ethyl-1-{3-methyl4-[4-(1 H-tetrazol-5-yl)-butoxy]-phenyl}-propyl)-2-methyl-phenyl]-1,1,1-trifluoro-2-trifluoromethyl-but-3-yn-2-ol

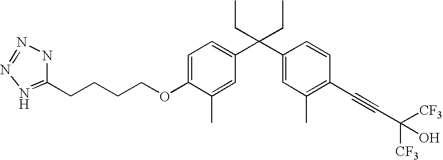

### (1) Preparation of 5-[4-(4-{1-ethyl-1-[3-methyl4-(4,4,4-trifluoro-3-methoxymethoxy-3-trifluoromethyl-but-1-ynyl)-phenyl]-propyl}-2-methyl-phenoxy)-butyl]-1H-tetrazole

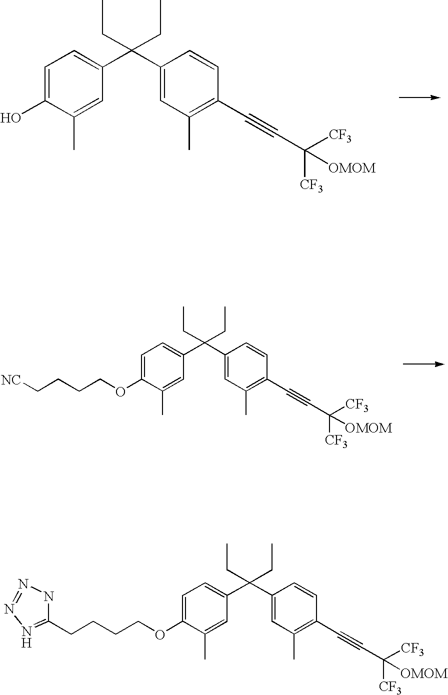

Using the same procedure as described for the preparation of Example 109-(1), 5-(4-{1-Ethyl-1-[3-methyl4-(4,4,4-trifluoro-3-methoxymethoxy-3-trifluoromethyl-but-1-ynyl)-phenyl]-propyl}-2-methyl-phenoxy)-pentanenitrile was prepared from 4-{1-Ethyl-1-[3-methyl4-(4,4,4-trifluoro-3-methoxymethoxy-3-trifluoromethyl-but-1-ynyl)-phenyl]-propyl}-2-methyl-phenol (compound prepared in Example 2-(2)). The yield was 99%.

Using the same procedure as described for the preparation of Example 109-(2), the title compound was prepared from 5-(4-{1-Ethyl-1-[3-methyl4-(4,4,4-trifluoro-3-methoxymethoxy-3-trifluoromethyl-but-1-ynyl)-phenyl]-propyl}-2-methyl-phenoxy)-pentanenitrile. The yield was 66%.

¹H-NMR (300 MHz, CDCl₃): 7.40 (d, 1 H), 7.05 (bs, 1 H), 7.00-6.85 (m, 2H), 6.82 (bs, 1H), 6.65 (d, 1H), 5.15 (s, 2H), 4.00 (t, 2H), 3.50 (s, 3H), 3.15 (t, 2H), 2.40 (s, 3H), 2.15 (s, 3H), 2.15-2.00 (m, 6H), 2.00-1.80 (m, 2H), 0.60 (t, 6H).

### (2) Preparation of 4-[4-(1-ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-yn-2-ol

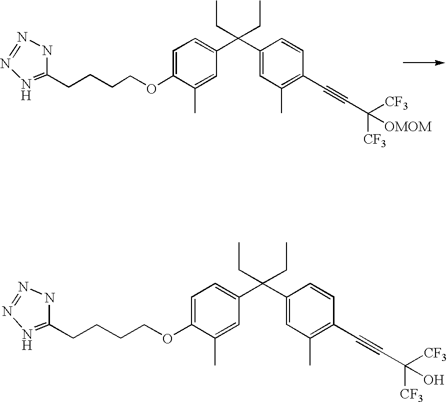

To a solution of 5-[4-(4-{1-ethyl-1-[3-methyl4-(4,4,4-trifluoro-3-methoxymethoxy-3-trifluoromethyl-but-1-ynyl)-phenyl]-propyl}-2-methyl-phenoxy)-butyl]-1H-tetrazole (compound prepared in Example 114-(1)) (21.0 mg, 0.03 mmol) in MeOH (0.34 ml) was added pTsOH (9.70 mg, 0.05 mmol) and the mixture was stirred at 60 degrees C. for 12 h. The reaction mixture was concentrated under reduced pressure and the obtained residue was purified by silica gel chromatography (CH₂Cl₂:MeOH═30:1) to give the title compound (16.0 mg, 80.8%) as colorless sticky oil

¹H-NMR (300 MHz, CDCl₃): 7.30 (d, 1H), 7.05 (bs, 1H), 7.00-6.80 (m, 3H), 6.65 (d, 1H), 3.98 (t, 2H), 3.10 (t, 2H), 2.35 (s, 3H), 2.15 (s, 3H), 2.15-2.00 (m, 6H), 2.00-1.80 (m, 2H), 0.60 (t, 6H); MS (ESI−): 581 ([M−H]⁻).

### Example 115

### Preparation of (E)4-[4-(1-Ethyl-1-{3-methyl4-[4-(1 H-tetrazol-5-yl)-butoxy]-phenyl}-propyl)-2-methyl-phenyl]-1,1,1-trifluoro-2-trifluoromethyl-but-3-en-2-ol

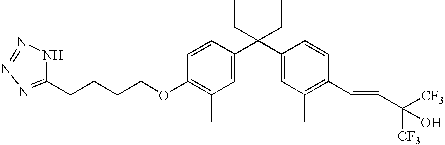

### (1) Preparation of 5-(4-{1-Ethyl-1-[3-methyl4-((E)4,4,4-trifluoro-3-methoxymethoxy-3-trifluoromethyl-but-1-enyl)-phenyl]-propyl}-2-methyl-phenoxy)-pentanenitrile

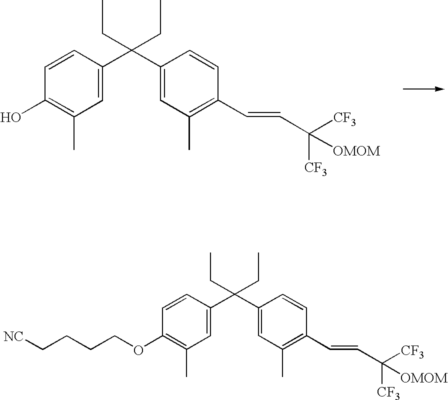

Using the same procedure as described for the preparation of Example 109-(1), the title compound was prepared from 4-{1-Ethyl-1-[3-methyl-4-((E)4,4,4-trifluoro-3-methoxymethoxy-3-trifluoromethyl-but-1-enyl)-phenyl]-propyl}-2-methyl-phenol (compound prepared in Example 30-(1)). The yield was 90%.

¹H-NMR (300 MHz, CDCl₃): 0.61 (t, 6H), 1.83-2.04 (m, 4H), 2.05 (q, 4H), 2.15 (s, 3H), 2.31 (s, 3H), 2.41 (t, 2H), 3.50 (s, 3H), 3.98 (t, 2H), 4.97 (s, 2H), 6.06 (d, 1H), 6.66 (d,1H), 6.89-7.02 (m, 4H), 7.30-7.39 (m, 2H).

### (2) Preparation of 5-[4-(4-{1-Ethyl-1-[3-methyl4-((E)4,4,4-trifluoro-3-methoxymethoxy-3-trifluoromethyl-but-1-enyl)-phenyl]-propyl}-2-methyl-phenoxy)-butyl]-1H-tetrazole

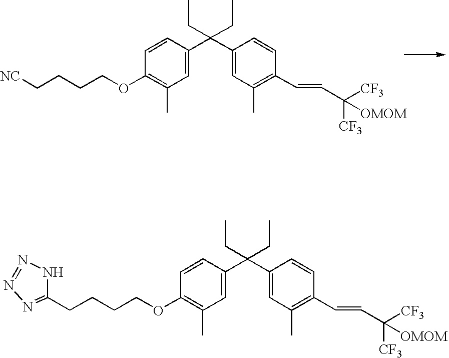

Using the same procedure as described for the preparation of Example 109-(2), the title compound was prepared from 5-(4-{1-ethyl-1-[3-methyl4-((E)-4,4,4-trifluoro-3-methoxymethoxy-3-trifluoromethyl-but-1-enyl)-phenyl]-propyl}-2-methyl-phenoxy)-pentanenitrile (compound prepared in Example 115-(1)). The yield was 60%.

¹H-NMR (300 MHz, CDCl₃): 0.61 (t, 6H), 1.83-2.04 (m, 4H), 2.05 (q, 4H), 2.15 (s, 3H), 2.32 (s, 3H), 3.11 (m, 2H), 3.50 (s, 3H), 4.00 (t, 2H), 4.96 (s, 2H), 6.06 (d, 1H), 6.69 (d,1 H), 6.89-7.02 (m, 4H), 7.30-7.39 (m, 2H).

### (3) Preparation of (E)-4-[4-(1-ethyl-1-{3-methyl-4-[4-(1 H-tetrazol-5-yl)-butoxy]-phenyl}-propyl)-2-methyl-phenyl]-1,1,1-trifluoro-2-trifluoromethyl-but-3-en-2-ol

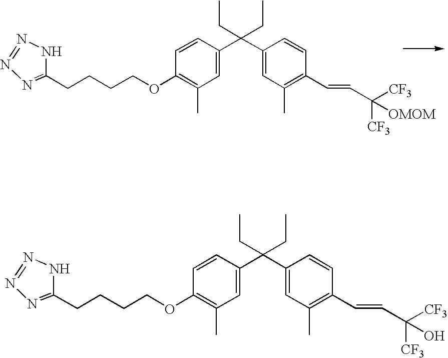

Using the same procedure as described for the preparation of Example 114-(2), the title compound was prepared from 5-[4-(4-{1-ethyl-1-[3-methyl4-((E)-4,4,4-trifluoro-3-methoxymethoxy-3-trifluoromethyl-but-1-enyl)-phenyl]-propyl}-2-methyl-phenoxy)-butyl]-1 H-tetrazole (compound prepared in Example 115-(2)). The yield was 61%.

¹H-NMR (300 MHz, CDCl₃): 0.60 (t, 6H), 1.83-2.04 (m, 4H), 2.05 (q, 4H), 2.14 (s, 3H), 2.32 (s, 3H), 3.12 (t, 2H), 4.00 (t, 2H), 6.08.(d, 1H), 6.68 (d, 1H), 6.89-7.00 (m, 4H), 7.30-7.39 (m, 2H); MS (ESI−): 583 ([M−H]⁻).

### Example 116

### Preparation of 1-[4-(1-ethyl-1-{3-methyl-4-[4-(1H-tetrazol-5-yl)-butoxy]-phenyl}-propyl)-2-methyl-phenyl]-4,4-dimethyl-pentan-3-ol

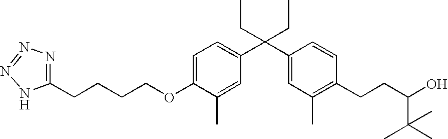

### (1) Preparation of 5-(4-{1-ethyl-1-[4-(3-hydroxy-4,4-dimethyl-pentyl)-3-methyl-phenyl]-propyl}-2-methyl-phenoxy)-pentanenitrile

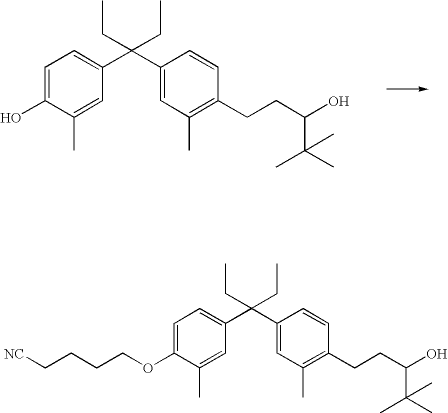

Using the same procedure as described for the preparation of Example 109-(1), the title compound was prepared from 4-{1-ethyl-1-[4-(3-hydroxy4,4-dimethyl-pentyl)-3-methyl-phenyl]-propyl}-2-methyl-phenol (compound prepared in Example 18). The yield was 98%.

¹H-NMR (300 MHz, CDCl₃): 0.59 (t, 6H), 0.89 (s, 9H), 1.38 (m, 1H), 1.43-1.53 (m, 1 H), 1.74-1.97 (m, 5H), 2.04 (q, 4H), 2.15 (s, 3H), 2.25 (s, 3H), 2.46 (d, 2H), 2.52-2.60 (m,1 H), 2.81-2.91 (m, 1H), 3.24 (d,1 H), 3.97 (t, 2H), 6.65 (d,1H), 6.90-6.96 (m, 4H), 7.01 (d,1H).

### (2) Preparation of 1-[4-(1-ethyl-1-{3-methyl4-[4-(1 H-tetrazol-5-yl)-butoxy]-phenyl}-propyl)-2-methyl-phenyl]-4,4-dimethyl-pentan-3-ol

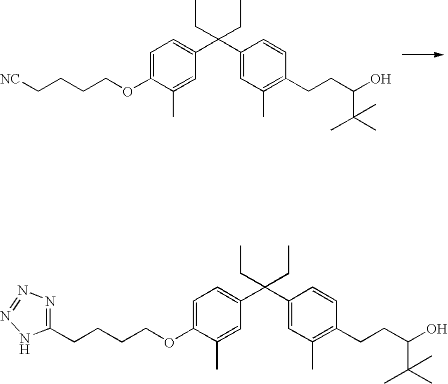

Using the same procedure as described for the preparation of Example 109-(2), the title compound was prepared from 5-(4-{1-ethyl-1-[4-(3-hydroxy4,4-dimethyl-pentyl)-3-methyl-phenyl]-propyl}-2-methyl-phenoxy)-pentanenitrile (compound prepared in Example 116-(1)). The yield was 65%.

¹H-NMR (300 MHz, CDCl₃): 0.59 (t, 6H),-0.89 (s, 9H), 1.38 (m,₁ H), 1.44-1.58 (m, 1H), 1.76-1.91 (m, 3H), 1.99-2.08 (m, 6H), 2.13 (s, 3H), 2.25 (s, 3H), 2.52-2.62 (m,1 H), 2.80-2.89 (m,1 H), 3.08 (t, 2H), 3.26 (dd,1 H), 3.98 (t, 2H), 6.65 (d, 1H), 6.89-6.95 (m, 4H), 7.01 (d, 1H); MS (ESI−): 505 ([M−H]⁻).

### Example 117

### Preparation of 1-[4-(1-ethyl-1-{3-methyl-4-[5-(1H-tetrazol-5-yl)-pentyloxy]-phenyl}-propyl)-2-methyl-phenyl]-4,4-dimethyl-pentan-3-ol

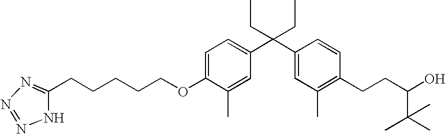

### (1) Preparation of 6-(4l1-ethyl-I -[4-(3-hydroxy4,4-dimethyl-pentyl)-3-methyl-phenyl]-propyl}-2-methyl-phenoxy)-hexanenitrile

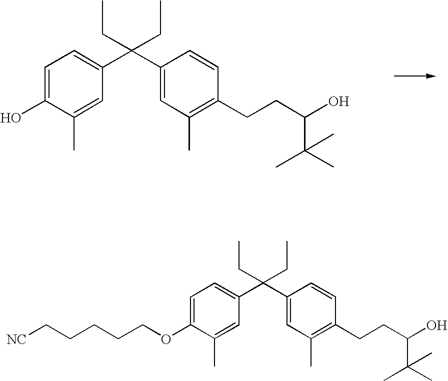

Using the same procedure as described for the preparation of Example 109-(1), the title compound was prepared from 4-{1-ethyl-1-[4-(3-hydroxy4,4-dimethyl-pentyl)-3-methyl-phenyl]-propyl}-2-methyl-phenol (compound prepared in Example 18) and 6-bromohexanenitrile. The yield was 89%.

¹H-NMR (300 MHz, CDCl₃): 0.59 (t, 6H), 0.89 (s, 9H), 1.41-1.87(m, 8H), 2.04 (q, 4H), 2.15 (s, 3H), 2.25 (s, 3H), 2.38 (t, 2H), 2.50-2.60 (m, 1H), 2.81-2.91 (m, 1H), 3.24 (dd, 1H), 3.94 (t, 2H), 6.65 (d, 1H), 6.89-6.95 (m, 4H), 7.01 (d, 1H).

### (2) Preparation of 1-[4-(1-Ethyl-1-{3-methyl4-[5-(1H-tetrazol-5-yl)-pentyloxy]-phenyl}-propyl)-2-methyl-phenyl]4,4-dimethyl-pentan-3-ol

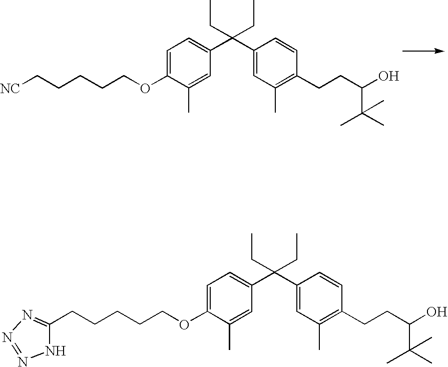

Using the same procedure as described for the preparation of Example 109-(2), the title compound was prepared from 6-(4-{1-Ethyl-1-[4-(3-hydroxy4,4-dimethyl-pentyl)-3-methyl-phenyl]-propyl}-2-methyl-phenoxy)-hexanenitrile (compound prepared in Example 117-(1)). The yield was 52%.

¹H-NMR (300 MHz, CDCl₃): 0.59 (t, 6H), 0.89 (s, 9H), 1.44-1.62 (m, 3H), 1.76-1.94 (m, 5H), 2.03 (q, 4H), 2.11 (s, 3H), 2.24 (s, 3H), 2.52-2.62 (m, 1H), 2.79-2.89 (m,1H), 2.99 (t, 2H), 3.26 (dd,1H), 3.92 (t, 2H), 6.63 (d,1H), 6.90-6.93 (m, 4H), 7.01 (d,1H); MS (ESI−): 519 ([M−H]⁻).

### Example 118

### Preparation of 1-(4-{1-[4-(5-amino-pentyloxy)-3-methyl-phenyl]-1-ethyl-propyl}-2-methyl-phenyl)-3-ethyl-pent-1-yn-3-ol

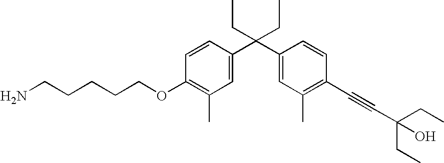

### (1) Preparation of 2-[5-(4-{1-ethyl-1-[4-(3-ethyl-3-hydroxy-pent-1-ynyl)-3-methyl-phenyl]-propyl}-2-methyl-phenoxy)-pentyl]-isoindole-1,3-dione

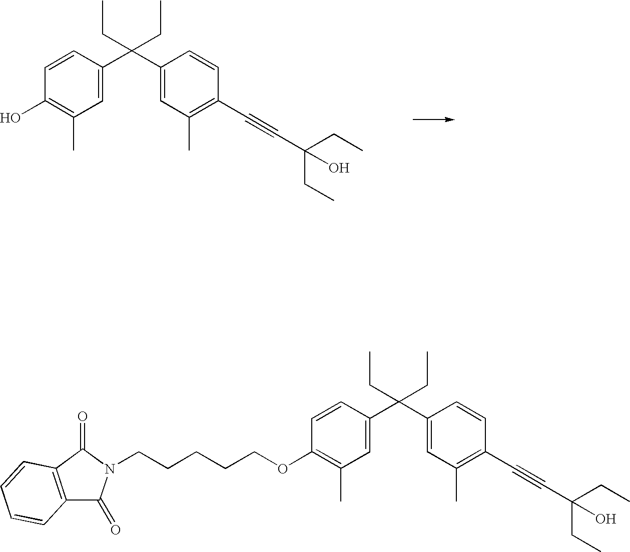

Using the same procedure as described for the preparation of Example 102-(1), the title compound was prepared from 4-{1-ethyl-1-[4-(3-ethyl-3-hydroxy-pent-1-ynyl)-3-methyl-phenyl]-propyl}-2-methyl-phenol (compound prepared in Example 1-(4)). The yield was 57%.

¹H NMR (CDCl₃): 7.84 (m, 2H), 7.70 (m, 2H), 7.26 (m, 1H), 7.00 (m, 1H), 6.91 (m, 2H), 6.82 (m,1H), 6.64 (d,1H), 3.91 (t, 2H), 3.70 (t, 2H), 2.36 (s, 3H), 2.09 (s, 3H), 2.04 (q, 4H), 1.91 (m, 4H), 1.75 (m, 4H), 1.25 (t, 2H), 1.10 (t, 6H), 0.58 (t, 6H).

### (2) Preparation of 1-(4-{1-[4-(5-amino-pentyloxy)-3-methyl-phenyl]-1-ethyl-propyl}-2-methyl-phenyl)-3-ethyl-pent-1-yn-3-ol

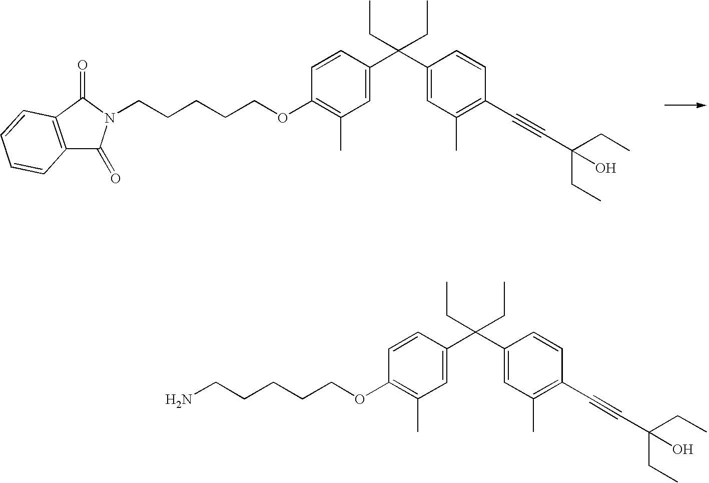

To a solution of 2-[5-(4-{l-ethyl-1-[4-(3-ethyl-3-hydroxy-pent-1-ynyl)-3-methyl-phenyl]-propyl}-2-methyl-phenoxy)-pentyl]-isoindole-1,3-dione (compound prepared in Example 118-(1)) (90 mg, 0.15 mmol) in EtOH (1 ml) under nitrogen atmosphere was added methylamine (0.3 ml, 40 wt % in water) and the mixture was stirred at room temperature for 2 h. The reaction mixture was concentrated under reduced pressure. The obtained residue was chromatographed on silica gel with CH₂Cl₂:MeOH (5:1) to give the title compound (14 mg, 20%).

¹H NMR (CDCl₃): 7.27 (m,₁H), 7.00 (m,1H), 6.90 (m, 2H), 6.84 (m,1H), 6.65 (d, 1H), 3.92 (t, 2H), 2.74 (m, 2H), 2.56 (br, NH2), 2.37 (s, 3H), 2.13 (s, 3H), 2.04 (q, 4H), 1.75 (m, 6H), 1.10 (t, 6H), 0.58 (t, 6H); MS (ESI+):464 ([M+H]⁺).

### Example 119

### Preparation of (E)-1-(4-{1-[4-(5-Amino-pentyloxy)-3-methyl-phenyl]-1-ethyl-propyl}-2-methyl-phenyl )-3-ethyl-pent-1-en-3-ol

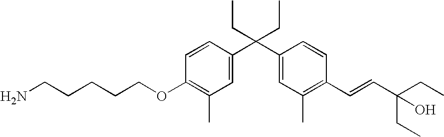

### (1)Preparation of 2-[5-(4-{1-Ethyl-1-[4-((E)-3-ethyl-3-hydroxy-pent-1-enyl)-3-methyl-phenyl]-propyl}-2-methyl-phenoxy)-pentyl]-isoindole-1,3-dione

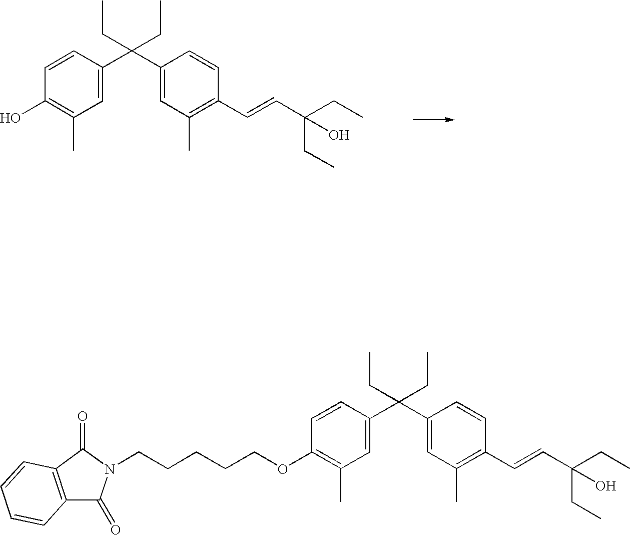

Using the same procedure as described for the preparation of Example 102-(1), the title compound was prepared from 4-{l-Ethyl-1-[4-((E)-3-ethyl-3-hydroxy-pent-1-enyl)-3-methyl-phenyl]-propyl}-2-methyl-phenol (compound prepared in Example 1-(5)). The yield was 34%.

¹H NMR (CDCl₃): 7.84 (m, 2H), 7.70 (m, 2H), 6.93 (m, 5H), 6.74 (d,1H), 6.64 (d, 1H), 6.00 (d, 1H), 3.91 (t, 2H), 3.72 (t, 2H), 2.30 (s, 3H), 2.10 (s, 3H), 2.04 (q, 4H), 1.79 (m, 4H), 1.64 (q, 4H), 1.25 (t, 2H), 0.91 (t, 6H), 0.60 (t, 6H).

### (2) Preparation of (E)-1-(4-{1-[4-(5-Amino-pentyloxy)-3-methyl-phenyl]-1-ethyl-propyl}-2-methyl-phenyl)-3-ethyl-pent-1-en-3-ol

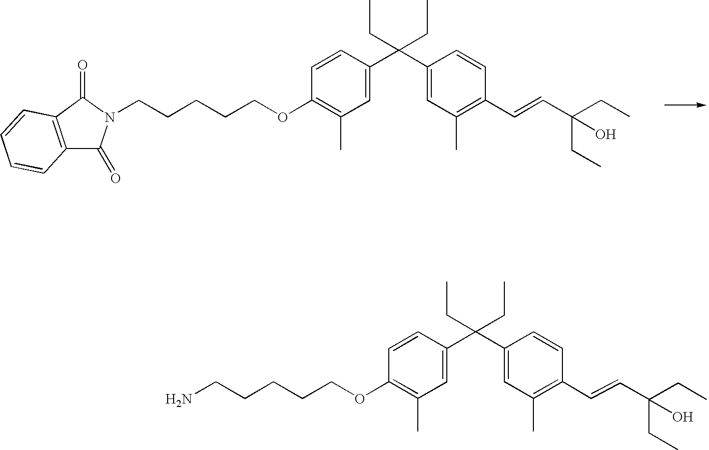

Using the same procedure as described for the preparation of Example 11 8-(2), the title compound was prepared from 2-[5-(4-{1-Ethyl-1-[4-((E)-3-ethyl-3-hydroxy-pent-1-enyl)-3-methyl-phenyl]-propyl}-2-methyl-phenoxy)-pentyl]-isoindole-1,3-dione (compound prepared in Example 119-(1)). The yield was 24%.

¹H NMR (CDCl₃): 7.29 (m,1H), 6.91 (m, 4H), 6.74 (d,1H), 6.63 (d, 1H), 6.00 (d, 1H), 3.87 (t, 2H), 3.75 (br, NH2), 2.74 (m, 2H), 2.29 (s, 3H), 2.13 (s, 3H), 2.04 (q, 4H), 1.74 (m, 2H), 1.66-1.46 (m, 8H), 0.91 (t, 6H), 0.60 (t, 6H); MS (ESI+) 466 ([M+H]⁺).

### Example 120

### Preparation of 1-(4-{1-[4-(5-Amino-pentyloxy)-3-methyl-phenyl]-1-ethyl-propyl}-2-methyl-phenylethynyl)-cyclopentanol

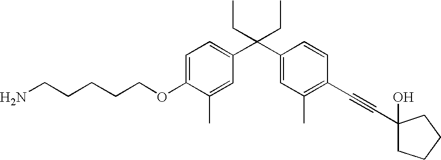

### (1) Preparation of 2-[5-(4-{1-Ethyl-1-[4-(1-hydroxy-cyclopentylethynyl)-3-methyl-phenyl]-propyl}-2-methyl-phenoxy)-pentyl]-isoindole-1,3-dione

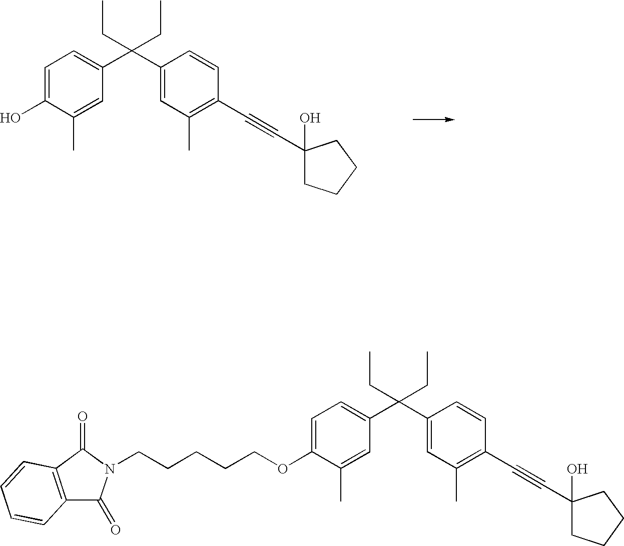

Using the same procedure as described for the preparation of Example 162-(1), the title compound was prepared from 4-{1-Ethyl-1-[4-(1-hydroxy-cyclopentylethynyl)-3-methyl-phenyl]-propyl}-2-methyl-phenol (compound prepared in Example 7). The yield was 68%.

¹H-NMR (300 MHz, CDCl₃): 0.58 (t, 6H), 1.47-1.58 (m, 4H), 1.72-1.88 (m, 6H), 1.98-2.05 (q, 8H), 2.09 (s, 3H), 2.35 (s, 3H), 3.72 (t, 2H), 3.91 (t, 2H), 6.64 (d, 1H), 6.81-7.00 (m, 5H), 7.68-7.71 (m, 2H), 7.82-7.85 (m, 2H).

### (2) Preparation of 1-(4-{1-[4-(5-Amino-pentyloxy)-3-methyl-phenyl]-1-ethyl-propyl}-2-methyl-phenylethynyl)-cyclopentanol

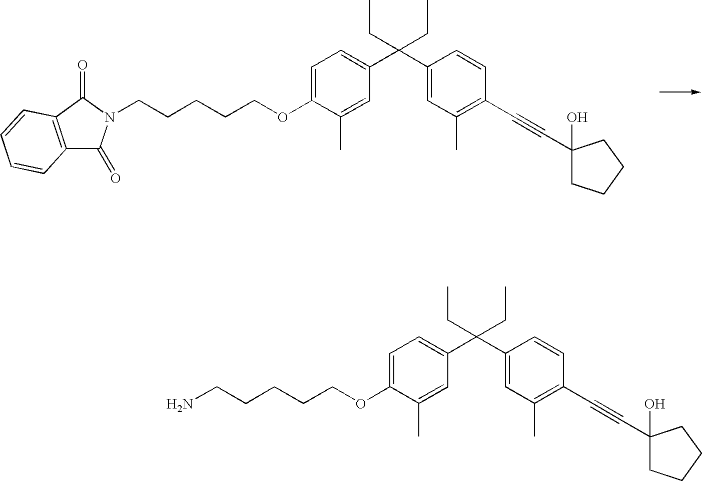

Using the same procedure as described for the preparation of Example 100-(2), the title compound was prepared from 2-[5-(4-{1-Ethyl-1-[4-(1-hydroxy-cyclopentylethynyl )-3-methyl-phenyl]-propyl}-2-methyl-phenoxy)-pentyl]-isoindole-1,3-dione (compound prepared in Example 120-(1)). The yield was 74%.

¹H-NMR (300 MHz, CDCl₃): 0.58 (t, 6H), 1.47-1.58 (m, 4H), 1.72-1.92 (m, 6H), 1.98-2.07 (q, 8H), 2.14 (s, 3H), 2.36 (s, 3H), 2.72 (t, 2H), 2.92 (t, 2H), 6.66 (d, 1H), 6.83-7.00 (m, 5H); MS (ESI+): 462 ([M+H]⁺).

### Example 121

### Preparation of 1-[(E)-2-(4-{1-[4-(5-Amino-pentyloxy)-3-methyl-phenyl]-1-ethyl-propyl)-2-methyl-phenyl )-vinyl]-cyclopentanol

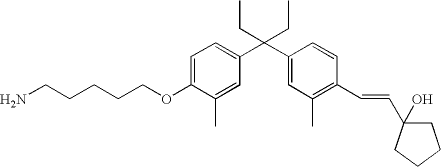

### (1) Preparation of 2-{5-[4-(1-Ethyl-1-{4-[(E)-2-(1-hydroxy-cyclopentyl)-vinyl]-3-methyl-phenyl}-propyl)-2-methyl-phenoxy]-pentyl}-isoindole-1,3-dione

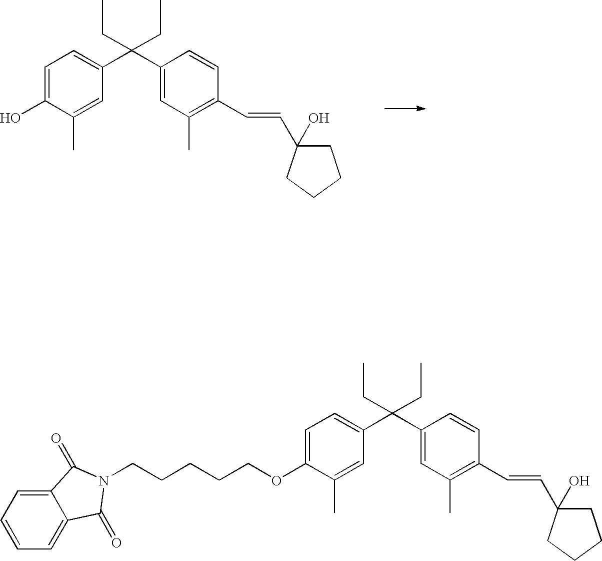

Using the same procedure as described for the preparation of Example 102-(1), the title compound was prepared from 4-(1-Ethyl-1-{4-[(E)-2-(1-hydroxy-cyclopentyl)-vinyl]-3-methyl-phenyl}-propyl)-2-methyl-phenol (compound prepared in Example 10). The yield was 63%.

¹H-NMR (300 MHz, CDCl₃): 0.59 (t, 6H), 1.47-1.88 (m, 14H), 2.02 (q, 4H), 2.10 (s, 3H), 2.38 (s, 3H), 3.72 (t, 2H), 3.92 (t, 2H), 6.66 (d, 1H), 6.82-7.00 (m, 4H), 7.27 (d,1H).

### (2) Preparation of 1-[(E)-2-(4-{1-[4-(5-Amino-pentyloxy)-3-methyl-phenyl]-1-ethyl-propyl}-2-methyl-phenyl)-vinyl]-cyclopentanol

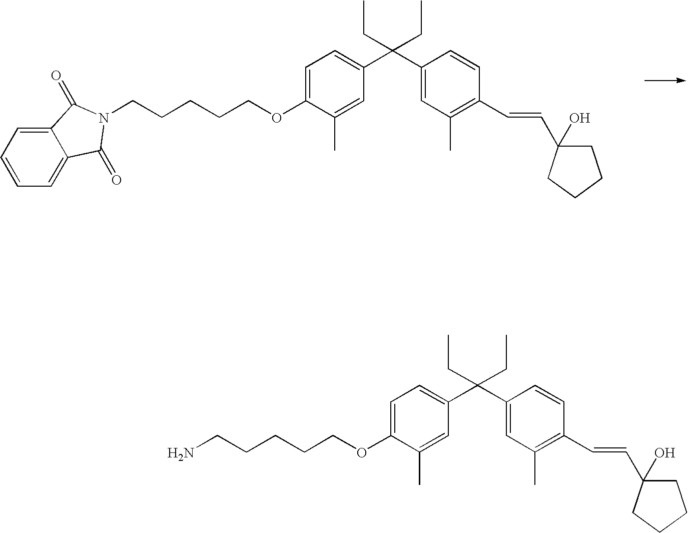

Using the same procedure as described for the preparation of Example 11 8-(2), the title compound was prepared from 2-{5-[4-(1-Ethyl-1-{4-[(E)-2-(1-hydroxy-cyclopentyl)-vinyl]-3-methyl-phenyl}-propyl)-2-methyl-phenoxy]-pentyl}-isoindole-1,3-dione (compound prepared in Example 121-(1)). The yield was 49%.

¹H-NMR (300 MHz, CDCl₃): 0.60 (t, 6H), 1.52 (m 4H), 1.70-1.99 (m, 10H), 2.04 (q, 4H), 2.15 (s, 3H), 2.31 (s, 3H), 2.72 (t, 2H), 3.93 (d, 2H), 6.25 (d, 1H), 6.67 (d, 1H), 6.68-6.96 (m, 5H), 7.31-7.34 (m, 1H); MS (ESI+): 464 ([M+H]⁺).

### Example 122

### Preparation of 1-(4-{1-[4-(5-Amino-pentyloxy)-3-methyl-phenyl]-1-ethyl-propyl}-2-methyl-phenylethynyl)-cyclohexanol

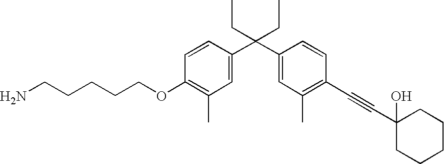

### (1) Preparation of 2-[5-(4-{1-Ethyl-1-[4-(1-hydroxy-cyclohexylethynyl)-3-methyl-phenyl]-propyl}-2-methyl-phenoxy)-pentyl]-isoindole-1,3-dione

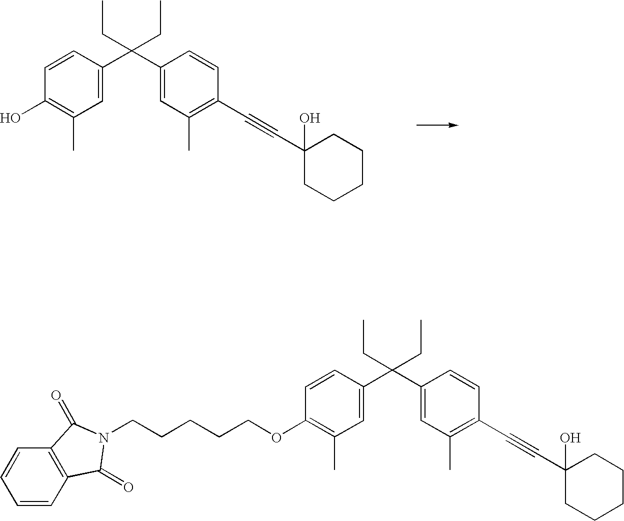

Using the same procedure as described for the preparation of Example 102-(1), the title compound was prepared from 4-{1-Ethyl-1-[4-(1-hydroxy-cyclohexylethynyl)-3-methyl-phenyl]-propyl}-2-methyl-phenol (compound prepared in Example 8). The yield was 83%.

¹H-NMR (300 MHz, CDCl₃): 0.59 (t, 6H), 1.47-1.88 (m, 16H), 2.02 (q, 4H), 2.10 (s, 3H), 2.38 (s, 3H), 3.72 (t, 2H), 3.92 (t, 2H), 6.65 (d, 1H), 6.82-7.00 (m, 4H), 7.27 (d,1H), 7.71 (m, 2H), 7.84 (m, 2H).

### (2) Preparation of I-(4-11-[4-(5-Amino-pentyloxy)-3-methyl-phenyl]-1-ethyl-propyl}-2-methyl-phenylethynyl)-cyclohexanol

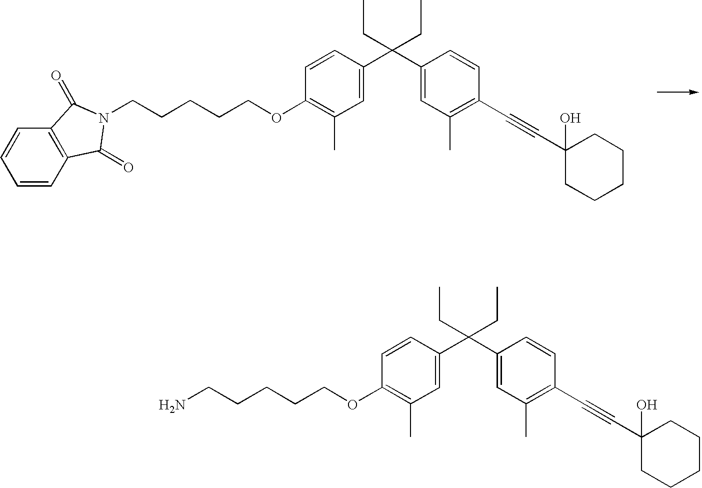

Using the same procedure as described for the preparation of Example 11 8-(2), the title compound was prepared from 2-[5-(4-{1-Ethyl-1-[4-(1-hydroxy-cyclohexylethynyl)-3-methyl-phenyl]-propyl}-2-methyl-phenoxy)-pentyl]-isoindole-1,3-dione (compound prepared in Example 122-(1)). The yield was 34%.

¹H-NMR (300 MHz, CDCl₃): 0.59 (t, 6H), 1.47-1.88 (m, 16H), 2.03 (q, 4H), 2.14 (s, 3H), 2.38 (s, 3H), 3.73 (m, 2H), 3.92 (t, 2H), 6.66 (d, 1H), 6.82-7.00 (m, 4H), 7.27 (d, 1H); MS (ESI+): 476 ([M+H]⁺).

### Example 123

### Preparation of 1-[(E)-2-(4-{1-[4-(5-Amino-pentyloxy)-3-methyl-phenyl]-1-ethyl-propyl}-2-methyl-phenyl)-vinyl]-cyclohexanol

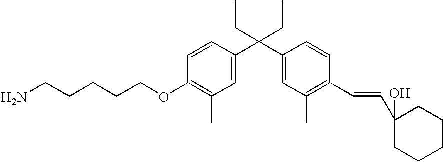

### (1) Preparation of 2-{5-[4-(1-Ethyl-1-{4-[(E)-2-(1-hydroxy-cyclohexyl)-vinyl]-3-methyl-phenyl}-propyl)-2-methyl-phenoxy]-pentyl}-isoindole-1,3-dione

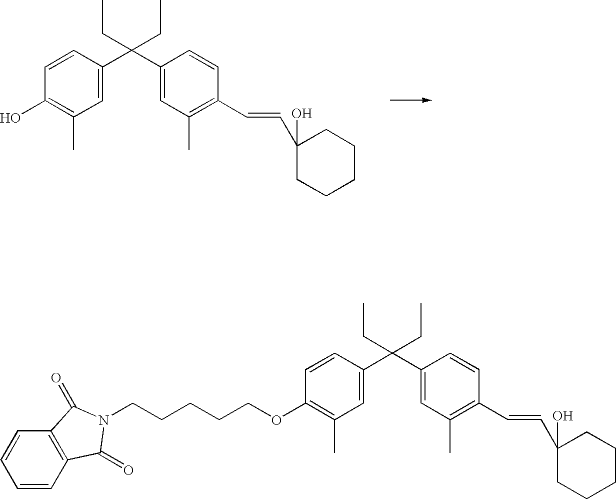

Using the same procedure as described for the preparation of Example 102-(1), the title compound was prepared from 4-(l-Ethyl-1-{4-[(E)-2-(l-hydroxy-cyclohexyl)-vinyl]-3-methyl-phenyl}-propyl)-2-methyl-phenol (compound prepared in Example 11). The yield was 89%.

¹H-NMR (300 MHz, CDCl₃): 0.60 (t, 6H), 1.32-1.85 (m, 16H), 2.03 (q, 4H), 2.10 (s, 3H), 2.30 (s, 3H), 3.72 (t, 2H), 3.91 (t, 2H), 6.20 (d,1H), 6.65 (d,1H), 6.78 -6.95 (m, 5H), 7.31 (d, 1H), 7.71 (m, 2H), 7.84 (m, 2H).

### (2) Preparation of 1-[(E)-2-(4-{1-[4-(5-Amino-pentyloxy)-3-methyl-phenyl]-1-ethyl-propyl}-2-methyl-phenyl)-vinyl]-cyclohexanol

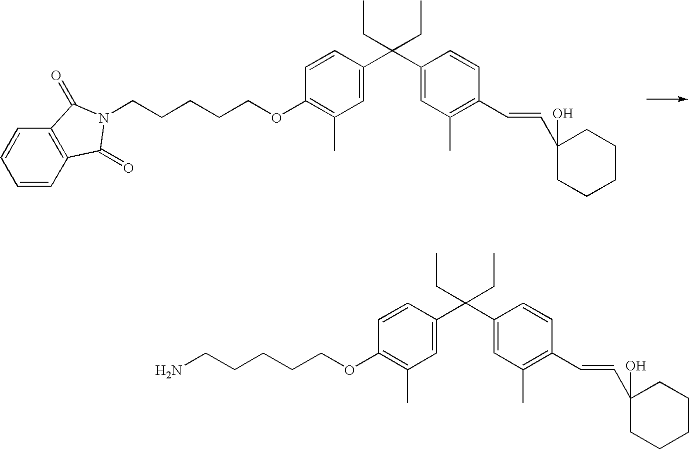

Using the same procedure as described for the preparation of Example 118-(2), the title compound was prepared from 2-{5-[4-(1-Ethyl-1-{4-[(E)-2-(1-hydroxy-cyclohexyl)-vinyl]-3-methyl-phenyl}-propyl)-2-methyl-phenoxy]-pentyl5-isoindole-1,3-dione (compound prepared in Example 123-(1)). The yield was 34%.

¹H-NMR (300 MHz, CDCl₃): 0.60 (t, 6H), 1.32-1.85 (m, 16H), 2.04 (q, 4H), 2.15 (s, 3H), 2.30 (s, 3H), 3.73 (m, 2H), 3.93 (t, 2H), 6.20 (d,1H), 6.66 (d,1H), 6.81 (d,1H), 6.89-6.95 (m, 4H), 7.32 (d,1H); MS (ESI+): 478 ([M+H]⁺).

### Example 124

### Preparation of 4-(4-{1-[4-(5-Amino-pentyloxy)-3-methyl-phenyl]-1-ethyl-propyl}-2-methyl-phenyl)-1,1,1-trifluoro-2-trifluoromethyl-but-3-yn-2-ol

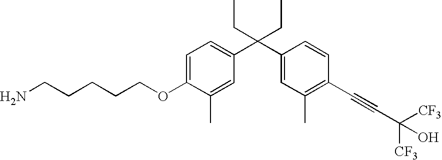

### (1) Preparation of 2-[5-(4-{1-Ethyl-1-[3-methyl4-(4,4,4-trifluoro-3-methoxymethoxy-3-trifluoromethyl-but-1-ynyl)-phenyl]-propyl}-2-methyl-phenoxy)-pentyl]-isoindole-1,3-dione

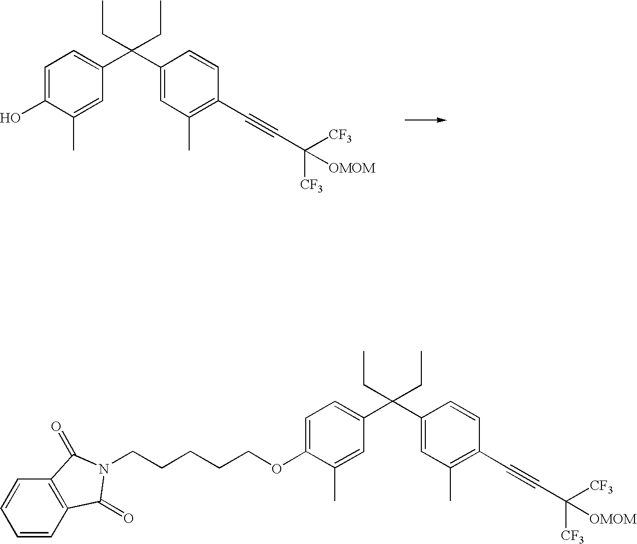

Using the same procedure as described for the preparation of Example 102-(1), the title compound was prepared from 4-{1-Ethyl-1-[3-methyl4-(4,4,4-trifluoro-3-methoxymethoxy-3-trifluoromethyl-but-1-ynyl)-phenyl]-propyl}-2-methyl-phenol (compound prepared in Example 2-(2)). The yield was 86%.

¹H-NMR (300 MHz, CDCl₃): 7.85 (m, 2H), 7.70 (m, 2H), 7.40 (d, 1H), 7.10-6.95 (m, 2H), 6.90-6.80 (m, 2H), 6.65 (d,1H), 5.15 (s, 2H), 3.95 (t, 2H), 3.75 (t, 2H), 3.50 (s, 3H), 2.40 (s, 3H), 2.10 (s, 3H), 2.05 (q, 4H), 1.90-1.70 (m, 4H), 1.60 -1.50 (m, 2H), 0.60 (t, 6H).

### (2) Preparation of 2-[5-(4-{1-Ethyl-1-[3-methyl4-(4,4,4-trifluoro-3-hydroxy-3-trifluoromethyl-but-1-ynyl)-phenyl]-propyl}-2-methyl-phenoxy)-pentyl]-isoindole-1,3-dione

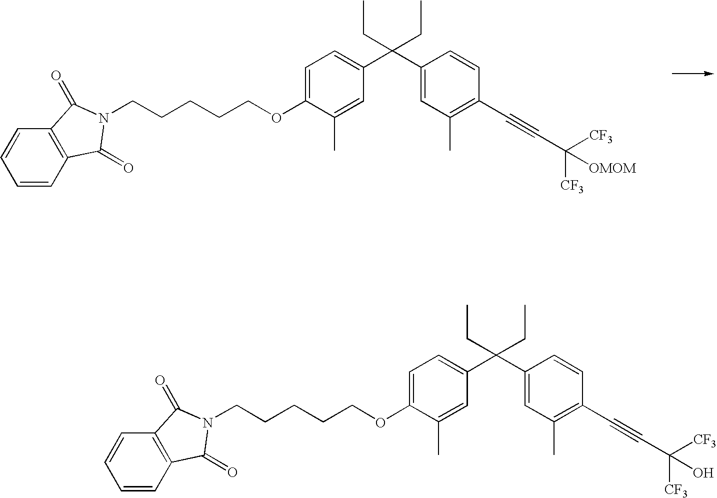

To a solution of 2-[5-(4-{1-Ethyl-1-[3-methyl4-(4,4,4-trifluoro-3-methoxymethoxy-3-trifluoromethyl-but-1-ynyl)-phenyl]-propyl}-2-methyl-phenoxy)-pentyl]-isoindole-1,3-dione (compound prepared in Example 124-(1)) (72.0 mg, 0.01 mmol) in MeOH/CH₂Cl₂(4/1 v/v, 2 ml) was added pTsOH(30.0 mg, 0.015 mmol) and the mixture was stirred at 60 degrees C. for 12 h. The reaction mixture was concentrated under reduced pressure and the obtained residue was purified by silica gel chromatography (n-Hexane:EtOAc=6:1) to give the title compound (61.5 mg, 91%) as colorless sticky oil.

¹H-NMR (300 MHz, CDCl₃): 7.85 (m, 2H), 7.70 (m, 2H), 7.40 (d, 1H), 7.10-6.95 (m, 2H), 6.90-6.80 (m, 2H), 6.65 (d, 1H), 4.10 (bs, 1H), 3.95 (t, 2H), 3.75 (t, 2H), 2.35 (s, 3H), 2.10 (s, 3H), 2.05 (q, 4H), 1.90-1.70 (m, 4H), 1.60-1.50 (m, 2H), 0.60 (t, 6H).

### (3) Preparation of 4-(4-{1-[4-(5-Amino-pentyloxy)-3-methyl-phenyl]-1-ethyl-propyl}-2-methyl-phenyl)-1,1,1-trifluoro-2-trifluoromethyl-but-3-yn-2-ol

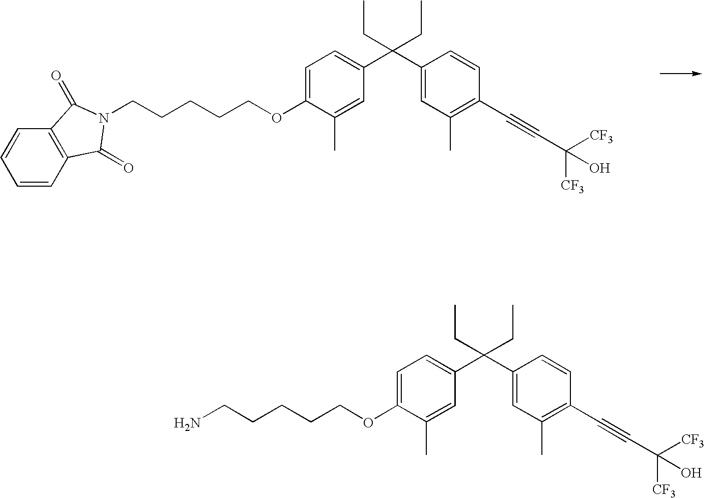

Using the same procedure as described for the preparation of Example 118-(2), the title compound was prepared from 2-[5-(4-{1-Ethyl-1-[3-methyl4-(4,4,4-trifluoro-3-hydroxy-3-trifluoromethyl-but-1-ynyl)-phenyl]-propyl}-2-methyl-phenoxy)-pentyl]-isoindole-1,3-dione (compound prepared in Example 124-(2)). The yield was 22%.

¹H-NMR (300 MHz, CDCl₃): 7.30 (d,1H), 7.05 (bs,1H), 7.00-6.80 (m, 3H), 6.65 (d, 1H), 4.70-4.50 (bs, 2H), 3.75 (t, 2H), 2.70 (t, 2H), 2.35 (s, 3H), 2.10 (s, 3H), 2.05 (q, 4H), 1.75-1.40 (m, 6H), 0.60 (t, 6H); MS (ESI−): 542 ([M−H]⁻).

### Example 125

### Preparation of (E)4-(4-{1-[4-(5-Amino-pentyloxy)-3-methyl-phenyl]-1-ethyl-propyl}-2-methyl-phenyl )-1,1,1-trifluoro-2-trifluoromethyl-but-3-en-2-ol

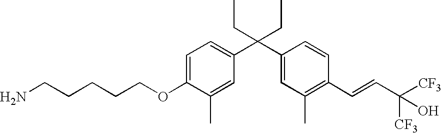

### (1) Preparation of 2-[5-(4-{1-Ethyl-1-[3-methyl4-((E)4,4,4-trifluoro-3-methoxymethoxy-3-trifluoromethyl-but-1-enyl)-phenyl]-propyl}-2-methyl-phenoxy)-pentyl]-isoindole-1,3-dione

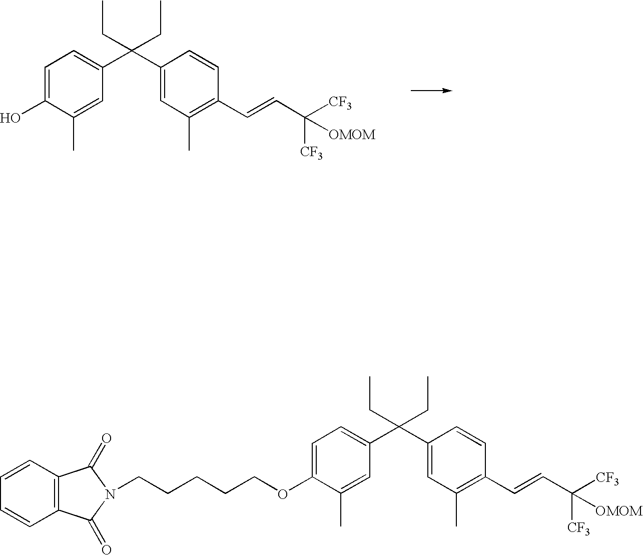

Using the same procedure as described for the preparation of Example 100-(1), the title compound was prepared from 4-{1-Ethyl-1-[3-methyl4-((E)-4,4,4-trifluoro-3-methoxymethoxy-3-trifluoromethyl-but-1-enyl)-phenyl]-propyl}-2-methyl-phenol (compound prepared in Example 30-(1)). The yield was 80%.

¹H-NMR (300 MHz, CDCl₃): 0.60 (t, 6H), 1.47-1.58 (m, 2H), 1.72-1.88 (m, 4H), 2.04 (q, 4H), 2.11 (s, 3H), 2.31 (s, 3H), 3.49 (s, 3H), 3.72 (t, 2H), 3.91 (t, 2H), 4.96 (s, 2H), 6.05 (d,1H), 6.65 (d,1H), 6.85-7.02 (m, 4H), 7.31-7.36 (m, 2H), 7.69-7.71 (m, 2H), 7.82-7.85 (m, 2H).

### (2) Preparation of 2-[5-(4-{1-Ethyl-1-[3-methyl4-((E)4,4,4-trifluoro-3-hydroxy-3-trifluoromethyl-but-1-enyl)-phenyl]-propyl}-2-methyl-phenoxy)-pentyl]-isoindole-1,3-dione

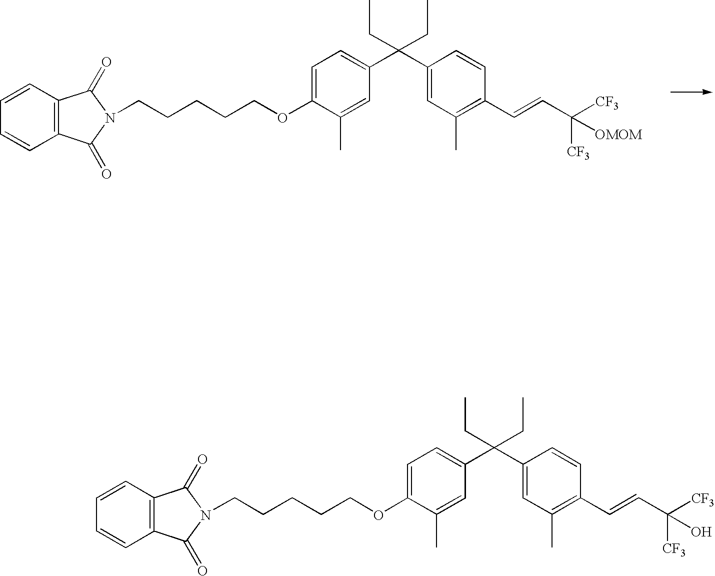

Using the same procedure as described for the preparation of Example 55 (deprotection procedure of MOM group with TMSBr), the title compound was prepared from 2-[5-(4-{1-Ethyl-1-[3-methyl4-((E)4,4,4-trifluoro-3-methoxymethoxy-3-trifluoromethyl-but-1-enyl)-phenyl]-propyl}-2-methyl-phenoxy)-pentyl]-isoindole-1,3-dione (compound prepared in Example 125-(1)). The yield was 95%.

¹H-NMR (300 MHz, CDCl₃): 0.59 (t, 6H), 1.47-1.58 (m, 2H), 1.70-1.88 (m, 4H), 2.03 (q, 4H), 2.10 (s, 3H), 2.32 (s, 3H), 3.71 (t, 2H), 3.90 (t, 2H), 6.07 (d, 1H), 6.64 (d,1H), 6.85-7.00 (m, 4H), 7.31-7.39 (m, 2H), 7.67-7.70 (m, 2H), 7.81-7.84 (m, 2H).

### (3) Preparation of (E)4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol

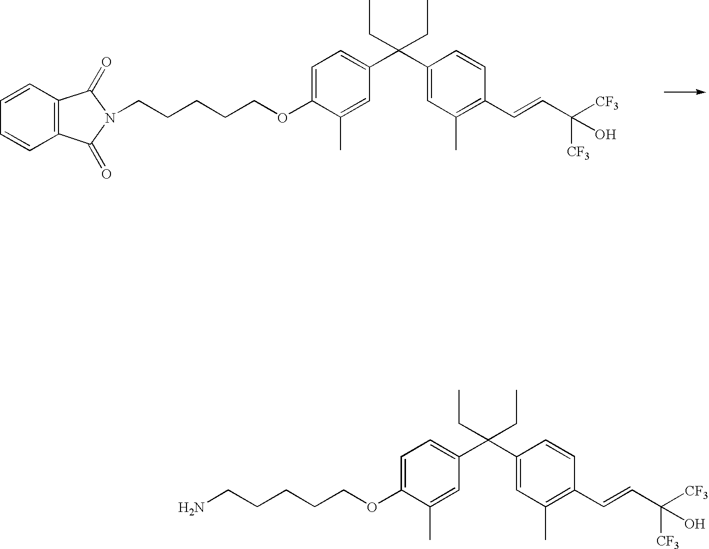

Using the same procedure as described for the preparation of Example 118-(2), the title compound was prepared from 2-[5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentyl]-isoindole-1,3-dione (compound prepared in Example 125-(2)). The yield was 32%.

¹H-NMR (300 MHz, CDCl₃): 0.60 (t, 6H), 1.47-1.58 (m, 4H), 1.73-1.81 (m, 2H), 2.04 (q, 4H), 2.13 (s, 3H), 2.31 (s, 3H), 3.71 (t, 2H), 3.90 (t, 2H), 6.08 (d, 1H), 6.64 (d,1H), 6.86-7.02 (m, 4H), 7.31-7.38 (m, 2H); MS (ESI+): 546 ([M+H]⁺).

### Example 126

### Preparation of 5(R)-(4-{1-Ethyl-1-[4-(3-hydroxy-4,4-dimethyl-pentyl)-3-methyl-phenyl]-propyl}-2-methyl-phenoxymethyl)-pyrrolidin-2-one

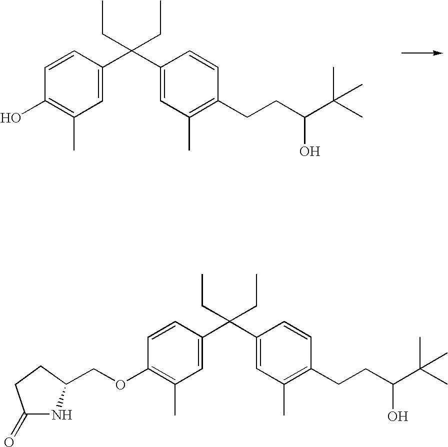

To a solution of 4-{1-Ethyl-1-[4-(3-hydroxy-4,4-dimethyl-phenyl)-3-methyl-phenyl]-propyl}-2-methyl-phenol (compound prepared in Example 18) (300 mg, 0.78 mmol) in anhydrous CH₃CN (7 ml) under nitrogen atmosphere were added K₂CO₃ (161 mg, 1.17 mmol) and (R)-toluene-4-sulfonic acid 5-oxo-pyrrolidin-2-ylmethyl ester (315 mg, 1.17 mmol) and the mixture was stirred at 100 degrees C. overnight. The reaction mixture was cooled to room temperature, poured into sat. NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:1) to give the title compound (200 mg, 53%).

¹H NMR (CDCl₃): 7.02 (d,1H), 6.93 (m, 4H), 6.63 (d,1H), 5.94 (s, NH), 4.08 (m, 1H), 3.96 (dd,1H), 3.80 (dd,1H), 3.23 (dt,1H), 2.86 (m, 1H), 2.55 (m,1H), 2.38 (m, 2H), 2.25 (s, 3H), 2.14 (s, 3H), 2.04 (q, 4H), 1.93 (m, 1H), 1.79 (m, 1H), 1.48 (m, 2H), 0.88 (s, 9H), 0.59 (t, 6H); MS (ESI+) 502 ([M+Na]⁺).

### Example 127

### Preparation of 5(S)-(4-{1-Ethyl-1-[4-(3-hydroxy4,4-dimethyl-pentyl)-3-methyl-phenyl]-propyl}-2-methyl-phenoxymethyl)-pyrrolidin-2-one

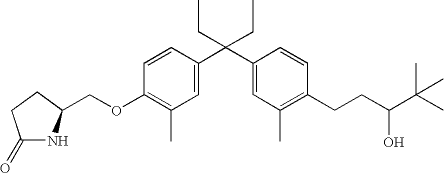

### Preparation of 5(S)-(4-{1-Ethyl-1-[4-(3-hydroxy4,4-dimethyl-pentyl)-3-methyl-phenyl]-propyl}-2-methyl-phenoxymethyl)-pyrrolidin-2-one

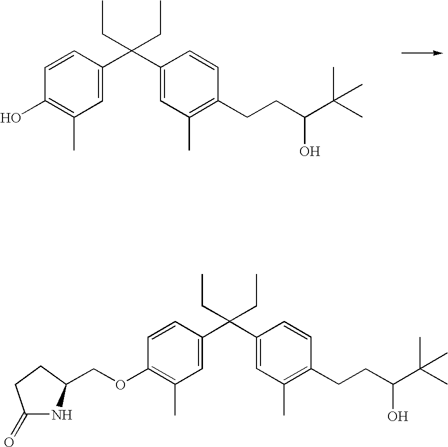

Using the same procedure as described for the preparation of Example 126, the title compound was prepared from 4-{1-Ethyl-1-[4-(3-hydroxy4,4-dimethyl-phenyl)-3-methyl-phenyl]-propyl}-2-methyl-phenol (compound prepared in Example 18) and (S)-toluene4-sulfonic acid 5-oxo-pyrrolidin-2-ylmethyl ester. The yield was 53%.

¹H NMR (CDCl₃): 7.02 (d,1H), 6.93 (m, 4H), 6.63 (d,1H), 5.94 (s, NH), 4.08 (m, 1H), 3.96 (dd,1H), 3.80 (dd,1H), 3.23 (dt,1H), 2.86 (m,1H), 2.55 (m, 1H), 2.38 (m, 2H), 2.25 (s, 3H:1), 2.14 (s, 3H), 2.04 (q, 4H), 1.93 (m, 1H), 1.79 (m, 1H), 1.48 (m, 2H), 0.88 (s, 9H), 0.59 (t, 6H); MS (ESI+): 502 ([M+Na]⁺).

### Example 128

### Preparation of 4(R)-Amino-5-(4-{1-ethyl-1-[4-(3-hydroxy4,4-dimethyl-pentyl)-3-methyl-phenyl]-propyl}-2-methyl-phenoxy)-pentanoic acid

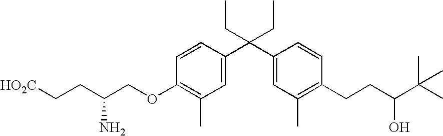

### Preparation of 4(R)-Amino-5-(4-{1-ethyl-1-[4-(3-hydroxy-4,4-dimethyl-pentyl)-3-methyl-phenyl]-propyl}-2-methyl-phenoxy)-pentanoic acid

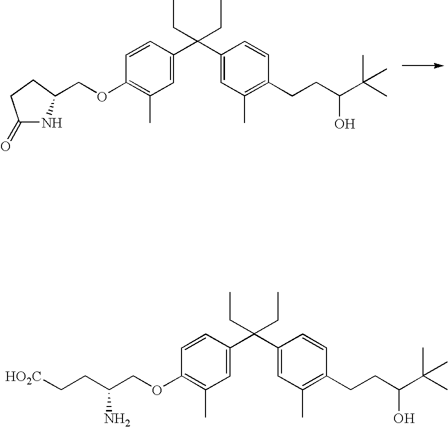

5(S)-(4-{1-Ethyl-I -[4-(3-hydroxy-4,4-dimethyl-pentyl)-3-methyl-phenyl]-propyl}-2-methyl-phenoxymethyl)-pyrrolidin-2-one (compound prepared in Example 126) (100 mg, 0.21 mmol) was dissolved in concentrated HCl (0.5 ml, 36wt % in water) and the mixture was stirred at reflux temperature overnight. The reaction mixture was concentrated under reduced pressure and the obtained residue was purified by ODS chromatography (CH₃OH/H₂O (0.1% TFA)=80/20) to give the title compound (25 mg, 24%).

¹H NMR (CD₃OD): 7.10-6.95 (m, 5H), 6.90 (d, 1H), 4.28 (dd, 1H), 4.14 (dd, 1H), 3.76 (m, 1H), 3.22 (dt, 1H), 2.93 (m, 1H), 2.60 (m, 3H), 2.30 (s, 3H), 2.27 (s, 3H), 2.14 (m, 6H), 1.82 (m,1H), 1.55 (m,1H), 0.94 (s, 9H), 0.65 (t, 6H); MS (ESI+): 498 ([M+H]⁺).

### Example 129

### Preparation of 4(S)-Amino-5-(4-{1-ethyl-1-[4-(3-hydroxy-4,4-dimethyl-pentyl)-3-methyl-phenyl]-propyl}-2-methyl-phenoxy)-pentanoic acid

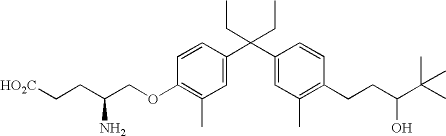

### Preparation of 4(S)-Amino-5-(4-{1-ethyl-1-[4-(3-hydroxy-4,4-dimethyl-pentyl)-3-methyl-phenyl]-propyl}-2-methyl-phenoxy)-pentanoic acid

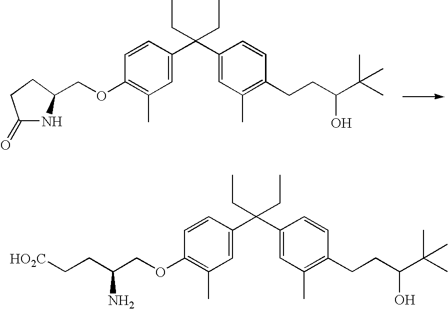

Using the same procedure as described for the preparation of Example 128, the title compound was prepared from 5(S)-(4-{1-Ethyl-1-[4-(3-hydroxy-4,4-dimethyl-pentyl)-3-methyl-phenyl]-propyl}-2-methyl-phenoxymethyl)-pyrrolidin-2-one (compound prepared in Example 127). The yield was 17%.

¹H NMR (CD₃OD): 7.10-6.95 (m, 5H), 6.90 (d, 1H), 4.28 (dd, 1H), 4.14 (dd, 1H), 3.76 (m,1H), 3.22 (dt,1H), 2.93 (m,1H), 2.60 (m, 3H), 2.30 (s, 3H), 2.27 (s, 3H), 2.14 (m, 6H), 1.82 (m, 1H), 1.55 (m, 1H), 0.94 (s, 9H), 0.65 (t, 6H); MS (ESI+): 498 ([M+H]⁺).

### Example 130

### Preparation of 1-(4-{1-[4-((S)-2-Amino-3-hydroxy-propoxy)-3-methyl-phenyl]-1-ethyl-propyl}-2-methyl-phenyl)4,4-dimethyl-pentan-3-ol

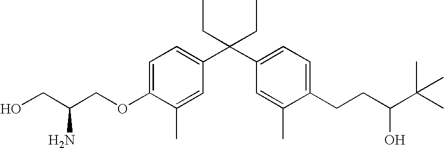

### (1) Preparation of (S)-2,2-Dimethyl4-(toluene4-sulfonyloxymethyl)-oxazolidine-3-carboxylic acid tert-butyl ester

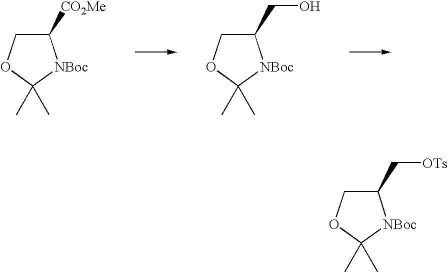

To a solution of (S)-2,2-Dimethyl-oxazolidine-3,4-dicarboxylic acid 3-tert-butyl ester 4-methyl ester (435 mg, 1.678 mmol) in THF (5 ml) under nitrogen atmosphere was added 1N-LiAlH₄ (5 ml, 5.033 mmol) at degrees C. and the mixture was stirred at room temperature for 1 h. The mixture was quenched with water and 10% NaOH solution, filtered through celite. The filtrate was diluted with EtOAc and washed with brine, dried over MgSO₄, filtered, and concentration in vacuo. The obtained residue was purified by flash chromatography eluting with 5:1Hexane/Ethyl acetate to give the (R)-4-Hydroxymethyl-2,2-dimethyl-oxazolidine-3-carboxylic acid tert-butyl ester (383 mg, 99%) as a white solid. To a solution of (R)-4-Hydroxymethyl-2,2-dimethyl-oxazolidine-3-carboxylic acid tert-butyl ester (383 mg, 1.656 mmol) in anhydrous pyridine (4 ml) under nitrogen atmosphere was added tosyl chloride (631 mg, 3.312 mmol) at 0 degrees C. and the mixture was stirred for 24 h. The reaction mixture was concentrated under reduced pressure and diluted with EtOAc, washed with brine, dried over MgSO4, filtered, and concentrated. Recrystallization from EtOAc-n-hexane to give the title compound (554 mg, 87%).

¹H-NMR (300 MHz, CDCl₃): 1.40 (s, 9H), 1.54 (s, 3H), 2.45 (s, 3H), 3.78-4.20 (m, 5H), 7.40 (d, 2H), 7.81 (d, 2H); MS (ESI+): 408 ([M+Na]⁺).

### (2) Preparation of 4(R)-(4-{1-Ethyl-1-[4-(3-hydroxy-4,4-dimethyl-pentyl)-3-methyl-phenyl]-propyl}-2-methyl-phenoxymethyl)-2,2-dimethyl-oxazolidine-3-carboxylic acid tert-butyl ester

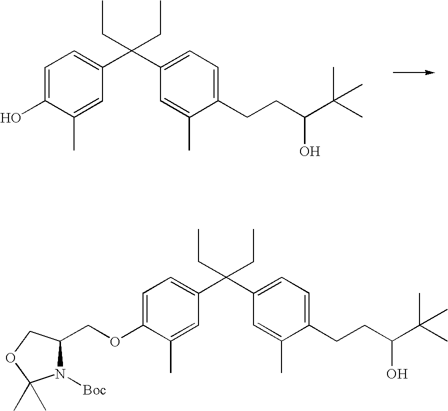

To a solution of 4-{1-Ethyl-1-[4-(3-hydroxy-4,4-dimethyl-pentyl)-3-methyl-phenyl]-propyl}-2-methyl-phenol (compound prepared in Example 18) (110 mg, 0.288 mmol) in anhydrous DMF (3 ml) under nitrogen atmosphere were added Cs₂CO₃ (281 mg, 0.864 mmol) and (S)-2,2-Dimethyl4-(toluene-4-sulfonyloxymethyl)-oxazolidine-3-carboxylic acid tert-butyl ester (compound prepared in Example 130-(1)) (167 mg, 0.432 mmol) and the mixture was stirred at 110 degrees C. for 3 h. The reaction mixture was cooled to room temperature, poured into sat. NH₄Cl aq. and the products were extracted with EtOAc. The extracts were washed with brine, dried over MgSO₄, filtered and concentrated under reduced pressure. The obtained residue was chromatographed on silica gel with EtOAc-n-Hexane (1:9) to give the title compound (169 mg, 77%) as a white solid.

¹H-NMR (300 MHz, CDCl₃): 0.60 (t, 6H), 0.89 (s, 9H), 1.43-1.84 (m, 5H), 2.04 (q, 4H), 2.16 (s, 3H), 2.25 (s,3H), 2.50-2.61 (m, 1H), 2.81-2.91 (m, 1H), 3.27 (dd, 1H), 3.78-3.93 (m,1H), 3.934.02 (m, 1H), 6.82 (d,1H), 6.90-6.97 (m, 4H), 7.01 (d, 1H).

### (3) Preparation of 1-(4-{1-[4-((S)-2-Amino-3-hydroxy-propoxy)-3-methyl-phenyl]-1-ethyl-propyl}-2-methyl-phenyl)4,4-dimethyl-pentan-3-ol

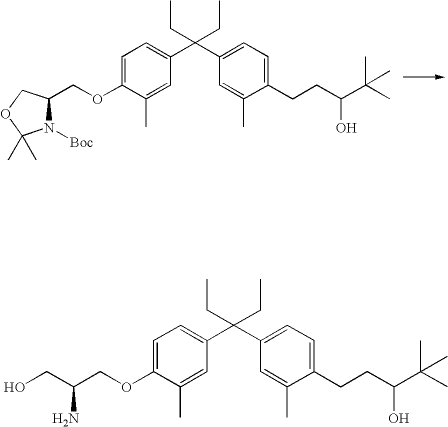

To a solution of 4(R)-(4-{1-Ethyl-1-[4-(3-hydroxy-4,4-dimethyl-pentyl)-3-methyl-phenyl]-propyl}-2-methyl-phenoxymethyl)-2,2-dimethyl-oxazolidine-3-carboxylic acid tert-butyl ester (compound prepared in Example 130-(2)) (148 mg, 0.248 mmol) in 1,4-dioxane (10 ml) was added conc.HCl (2 ml) at 0 degrees C. and the mixture was stirred at room temperature for 4 h. The mixture was neutralized by sat. NaHCO₃ solution, diluted with EtOAc, washed with brine, dried over MgSO₄, filtered, and concentration in vacuo. The obtained residue was chromatographed on amino functionalized silica gel with EtOAc to give the title compound (30 mg, 26.5%).

¹H-NMR (300 MHz, CDCl₃): 0.59 (t, 6H), 0.89 (s, 9H), 1.55 (m,1H), 1.75-1.84 (m, 1H), 2.04 (q, 4H), 2.16 (s, 3H), 2.25 (s,3H), 2.55-2.57 (m, 1H), 2.84-2.85 (m, 1H), 3.23 (d,1H), 3.25-3.40 (m,1H), 3.60 (dd,1H), 3.75 (dd,1H), 3.91-3.98 (m, 2H), 6.70 (d,1H), 6.90-6.97 (m, 4H), 7.02 (d,1H); MS (ESI+): 456 ([M+H]⁺).

### Example 131

### Preparation of 1-(4-{1-[4-((R)-2-Amino-3-hydroxy-propoxy)-3-methyl-phenyl]-1-ethyl-propyl}-2-methyl-phenyl)4,4-dimethyl-pentan-3-ol

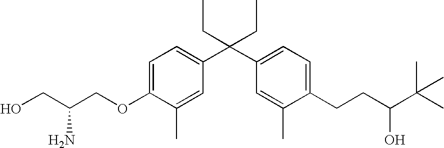

### (1) Preparation of (R)-2,2-Dimethyl-4-(toluene4-sulfonyloxymethyl)-oxazolidine-3-carboxylic acid tert-butyl ester

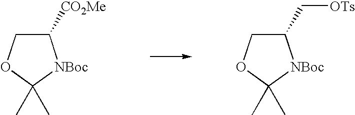

Using the same procedure as described for the preparation of Example 130-(1), the title compound was prepared from (R)-2,2-Dimethyl-oxazolidine-3,4-dicarboxylic acid 3-tert-butyl ester 4-methyl ester. The yield was 49%.

¹H-NMR (300 MHz, CDCl₃): 1.40 (m, 9H), 1.51 (s, 3H), 2.49 (s, 3H), 3.71-4.20 (m, 5H), 7.40 (d, 2H), 7.81 (d, 2H); MS (ESI+): 408 ([M+H]⁺).

### (2) Preparation of 4(S)-(4-{1-Ethyl-1-[4-(3-hydroxy4,4-dimethyl-pentyl )-3-methyl-phenyl]-propyl}-2-methyl-phenoxymethyl)-2,2-dimethyl-oxazolidine-3-carboxylic acid tert-butyl ester

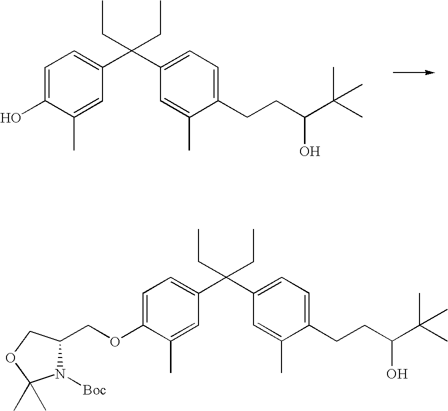

Using the same procedure as described for the preparation of Example 130-(2), the title compound was prepared from 4-{1-Ethyl-1-[4-(3-hydroxy4,4-dimethyl-pentyl)-3-methyl-phenyl]-propyl}-2-methyl-phenol (compound prepared in Example 18). The yield was 78%.

¹H-NMR (300 MHz, CDCl₃): 0.60 (t, 6H), 0.89 (s, 9H), 1.43-1.84 (m, 5H), 2.04 (q, 4H), 2.16 (s, 3H), 2.25 (s,3H), 2.50-2.61 (m, 1H), 2.81-2.91 (m, 1H), 3.27 (dd, 1H), 3.78-3.93 (m,1H), 3.934.02 (m,1H), 6.82 (d,1H), 6.90-6.97 (m, 4H), 7.01 (d, 1H).

### (3) Preparation of 1-(4-{1-[4-((R)-2-Amino-3-hydroxy-propoxy)-3-methyl-phenyl]-1-ethyl-propyl}-2-methyl-phenyl)-4,4-dimethyl-pentan-3-ol

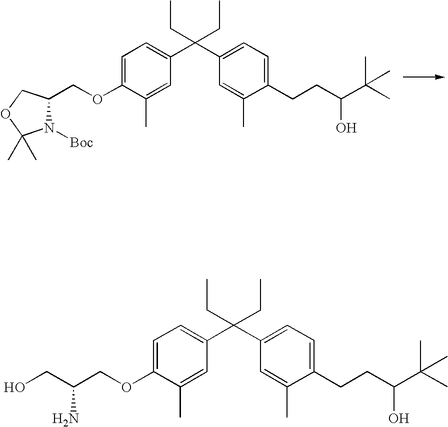

Using the same procedure as described for the preparation of Example 130-(3), the title compound was prepared from 4(S)-(4-{1-Ethyl-1-[4-(3-hydroxy4,4-dimethyl-pentyl)-3-methyl-phenyl]-propyl}-2-methyl-phenoxymethyl)-2,2-dimethyl-oxazolidine-3-carboxylic acid tert-butyl ester (compound prepared in Example 131-(2)). The yield was 59%.

¹H-NMR (300 MHz, CDCl₃): 0.60 (t, 6H), 0.89 (s, 9H), 1.43-1.57 (m, 1H), 1.68-1.84 (m, 4H), 2.04 (q, 4H), 2.16 (s, 3H), 2.25 (s,3H), 2.51-2.61 (m, 1H), 2.81-2.91 (m,1H), 3.22 (dd,1H), 3.25-3.40 (m,1H), 3.61 (dd,1H), 3.75 (dd,1H), 3.91-3.98 (m, 2H), 6.68 (d,1H), 6.90-6.97 (m, 4H), 7.01 (d,1H); MS (ESI+) 456 ([M+H]⁺).

### Example 132

### Preparation of 1 (R)-(4-{1-[4-((R)-2-Amino4-hydroxy-butoxy)-3-methyl-phenyl]-1-ethyl-propyl}-2-methyl-phenyl)4,4-dimethyl-pentan-3-ol

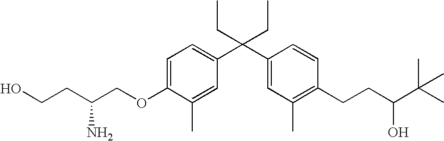

### (1) Preparation of (R)-1-(tert-Butyl-dimethyl-silanyl)4-hydroxymethyl-azetidin-2-one

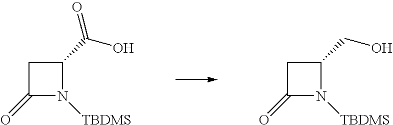

To a solution of (R)-1-(tert-Butyl-dimethyl-silanyl)-4-oxo-azetidine-2-carboxylic acid (800 mg, 3.49 mmol) in CH₂Cl₂ (30 ml) were added BnOH (0.4 ml, 3.84 mmol), EDC (803 mg, 4.19 mmol), and DMAP (catalytic amount) and the mixture was stirred at room temperature overnight. The reaction mixture was poured into water and the products were extracted with EtOAc. The extracts were washed with brine, dried over MgSO₄, filtered and concentrated under reduced pressure. The obtained, residue was chromatographed on silica gel with EtOAc-n-Hexane (1:40) to give (R)-1-(tert-Butyl-dimethyl-silanyl)-4-oxo-azetidine-2-carboxylic acid benzyl ester (954 mg, 86%).

To a solution of CaCl₂ (994 mg, 8.96 mmol) in EtOH (6 ml) and THF (12 ml) was added NaBH₄ (567 mg, 14.9 mmol) at room temperature. To the mixture, (R)-1-(tert-Butyl-dimethyl-silanyl)-4-oxo-azetidine-2-carboxylic acid benzyl ester (954 mg, 2.97 mmol) was added and the mixture was stirred at 0 degrees C. for 3 h. The reaction mixture was poured into water and the products were extracted with EtOAc. The extracts were washed with brine, dried over MgSO₄, filtered and concentrated under reduced pressure. The obtained residue was chromatographed on silica gel with EtOAc-n-Hexane (1:5) to give the title compound (150 mg, 23%).

¹H-NMR (300 MHz, CDCl₃): 0.23 (s, 3H), 0.26 (s, 3H), 0.94 (s, 9H), 1.74-1.84 (m, 1H), 2.84 (dd,1H), 3.08 (dd, 1H), 3.63-3.84 (m, 3H).

### (2) Preparation of 1-(tert-Butyl-dimethyl-silanyl)4(R)-(4-{1-ethyl-1-[4-(3-hydroxy-4,4-dimethyl-pentyl)-3-methyl-phenyl]-propyl}-2-methyl-phenoxymethyl)-azetidin-2-one

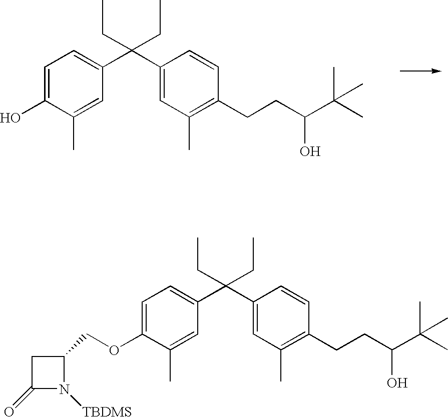

To a solution of 4-{1-Ethyl-1-[4-(3-hydroxy-4,4-dimethyl-pentyl)-3-methyl-phenyl]-propyl}-2-methyl-phenol (compound prepared in Example 18) (140 mg, 0.65 mmol) were added triphenylphosphine (256 mg, 0.975 mmol), DEAD (0.15 ml, 0.975 mmol), and (R)-1-(tert-Butyl-dimethyl-silanyl)-4-hydroxymethyl-azetidin-2-one (compound prepared in Example 132-(1)) (274 mg, 0.715 mmol) and the mixture was stirred at room temperature for 2 days. The mixture was filtered and concentrated under reduced pressure. The obtained residue was chromatographed on silica gel with EtOAc-n-Hexane (1:7) to give the title compound (186 mg, 49%).

¹H-NMR (300 MHz, CDCl₃): 0.23 (s, 3H), 0.26 (s, 3H), 0.59 (t, 6H), 0.89 (s, 9H), 0.94 (s, 9H), 1.39 (d, 1H), 1.48-1.57 (m, 1H), 1.74-1.84 (m, 1H), 2.03 (q, 4H), 2.12 (s, 3H), 2.25 (s, 3H), 2.49-2.69 (m,1H), 2.98 (m,1H), 3.31-3.27 (m, 2H), 3.89-3.95 (m, 1H), 4.08-4.22 (m, 2H), 6.63 (d, 1H), 6.89-6.97 (m, 4H), 7/01 (d, 1H); MS (ESI+): 602 ([M+Na]⁺).

### (3) Preparation of 2(R)-(4-{1-Ethyl-1-[4-(3-hydroxy-4,4-dimethyl-pentyl)-3-methyl-phenyl]-propyl}-2-methyl-phenoxymethyl)-4-oxo-azetidine-1-carboxylic acid tert-butyl ester

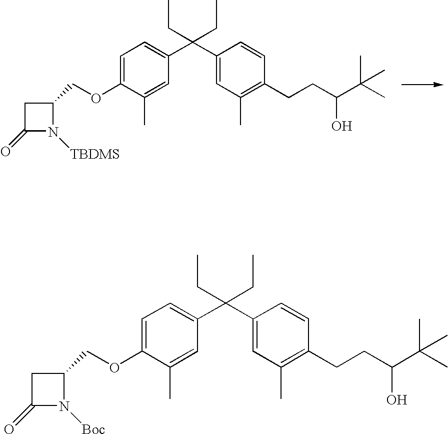

To a solution of 1-(tert-Butyl-dimethyl-silanyl)-4(R)-(4-{1-ethyl-1-[4-(3-hydroxy-4,4-dimethyl-pentyl)-3-methyl-phenyl]-propyl}-2-methyl-phenoxymethyl)-azetidin-2-one (compound prepared in Example 132-(2)) (184 mg, 0.317 mmol) in THF (3 ml) was added 1 M TBAF in THF (0.48 ml, 0.48 mmol) and the mixture was stirred at room temperature for 2 h. The reaction mixture was poured into water and the products were extracted with EtOAc. The extracts were washed with brine, dried over Na₂SO₄, filtered and concentrated under reduced pressure. The obtained residue was dissolved in CH₂Cl₂ (3 ml). To the mixture, DMAP (catalytic amount), triethylamine (0.13 ml, 0.96 mmol), and (Boc)₂0 (95 mg, 0.42 mmol) were added and the mixture was stirred at room temperature overnight. The reaction mixture was poured into water and the products were extracted with EtOAc. The extracts were washed with brine, dried over Na₂SO₄, filtered and concentrated under reduced pressure. The obtained residue was chromatographed on silica gel with EtOAc-n-Hexane (1:4) to give the title compound (127 mg, 71%).

¹H-NMR (300 MHz, CDCl₃): 0.59 (t, 6H), 0.89 (s, 9H), 1.47 (s, 9H), 1.48-1.57 (m, 1H), 1.74-1.84 (m, 1H), 2.03 (q, 4H), 2.14 (s, 3H), 2.25 (s, 3H), 2.50-2.60 (m, 1H), 2.81-2.91 (m, 1H), 3.12 (t, 2H), 3.24 (d, 1H), 4.194.29 (m, 2H), 4.38 (dd, 1H), 6.68 (d,1H), 6.89-6.97 (m, 4H), 7.01 (d,1H).

### (4) Preparation of [1(R)-(4-{1-Ethyl-1-[4-(3-hydroxy-4,4-dimethyl-pentyl)-3-methyl-phenyl]-propyl}-2-methyl-phenoxymethyl)-3-hydroxy-propyl]-carbamic acid tert-butyl ester

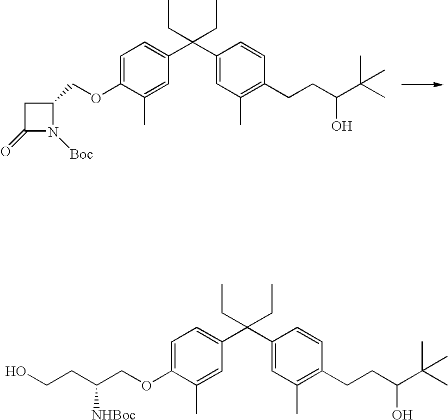

To a solution of 2(R)-(4-{1-Ethyl-1-[4-(3-hydroxy4,4-dimethyl-pentyl)-3-methyl-phenyl]-propyl}-2-methyl-phenoxymethyl)4-oxo-azetidine-1-carboxylic acid tert-butyl ester (compound prepared in Example 132-(3)) (127 mg, 0.224 mmol) in THF (2 ml) was added 1 M LAH in THF (0.33 ml, 0.33 mmol) and the mixture was stirred at 0 degrees C. for 30 min. The reaction mixture was quenched with water and 10% NaOH, dried over Na₂SO₄, filtered and concentrated under reduced pressure. The obtained residue was chromatographed on silica gel with EtOAc-n-Hexane (1:3) to give the title compound (114 mg, 87%).

¹H-NMR (300 MHz, CDCl₃): 0.59 (t, 6H), 0.89 (s, 9H), 1.45 (s, 9H), 1.48-1.57 (m, 1H), 1.73-1.89 (m, 3H), 2.03 (q, 4H), 2.16 (s, 3H), 2.25 (s, 3H), 2.50-2.60 (m, 1H), 2.81-2.91 (m,1H), 3.21-3.26 (m,1H), 3.36 (m,1H), 3.71 (m, 2H), 3.91-3.95 (dd,1H), 4.084.20 (m, 2H), 4.38 (dd,1H), 6.66 (d, 1H), 6.88-6.98 (m, 4H), 7.01 (d, 1H).

### (5) Preparation of 1((R(4-{1-[4-((R)-2-Amino-4-hydroxy-butoxy)-3-methyl-phenyl]-1-ethyl-propyl}-2-methyl-phenyl)4,4-dimethyl-pentan-3-ol

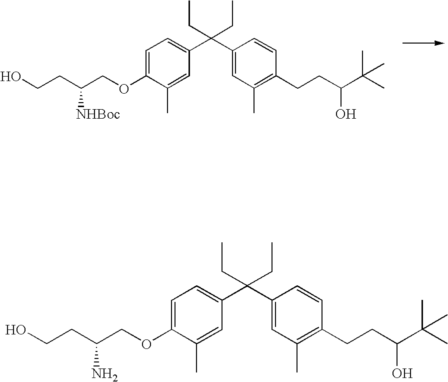

To a solution of [1 (R)-(4-{1-Ethyl-1-[4-(3-hydroxy4,4-dimethyl-pentyl)-3-methyl-phenyl]-propyl}-2-methyl-phenoxymethyl)-3-hydroxy-propyl]-carbamic acid tert-butyl ester (compound prepared in Example 132-(4)) (111 mg, 0.195 mmol) in dioxane (2.5 ml) was added conc. HCl (1.1 ml) and the mixture was stirred at room temperature for 1 h. The reaction mixture was poured into sat. NaHCO₃ aq. and the products were extracted with EtOAc. The extracts were washed with brine, dried over Na₂SO₄, filtered and concentrated under reduced pressure. The obtained residue was chromatographed on silica gel with CH₂Cl₂—MeOH (10:1) to give the title compound (50 mg, 54%).

¹H-NMR (300 MHz, CDCl₃): 0.59 (t, 6H), 0.89 (s, 9H), 1.43-1.57 (m, 1H), 1.70-1.89 (m, 3H), 2.03 (q, 4H), 2.17 (s, 3H), 2.25 (s, 3H), 2.50-2.60 (m, 1H), 2.81-2.91 (m,1H), 3.24 (d,1H), 3.31-3.40 (m,1H), 3.74 (m, 1H), 3.85-3.91 (m,2), 6.66 (d,1H), 6.88-6.98 (m, 4H), 7.01 (d,1H); MS (ESI+): 470 ([M+H]⁺).

### Example 133

### Preparation of 1 (S)-(4-{1-[4-((S)-2-Amino4-hydroxy-butoxy)-3-methyl-phenyl]-1-ethyl-propyl}-2-methyl-phenyl)4,4-dimethyl-pentan-3-ol

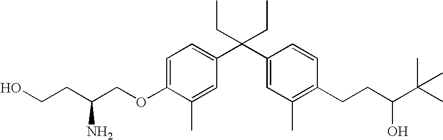

### (1) Preparation of (S)-1-(tert-Butyl-dimethyl-silanyl)4-hydroxymethyl-azetidin-2-one

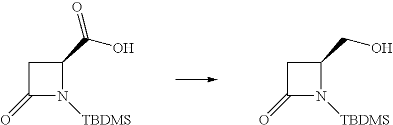

Using the same procedure as described for the preparation of Example 132-(1), the title compound was prepared from (S)-1-(tert-Butyl-dimethyl-silanyl)4-oxo-azetidine-2-carboxylic acid. The yield was 88%.

¹H-NMR (300 MHz, CDCl₃): 0.23 (s, 3H), 0.26 (s, 3H), 0.94 (s, 9H), 1.74-1.84 (m, 1H), 2.84 (dd,1H), 3.08 (dd,1H), 3.63-3.84 (m, 3H).

### (2) Preparation of 1-(tert-Butyl-dimethyl-silanyl)-4(S)-(4-{1-ethyl-1-[4-(3-hydroxy-4,4-dimethyl-pentyl)-3-methyl-phenyl]-propyl}-2-methyl-phenoxymethyl)-azetidin-2-one

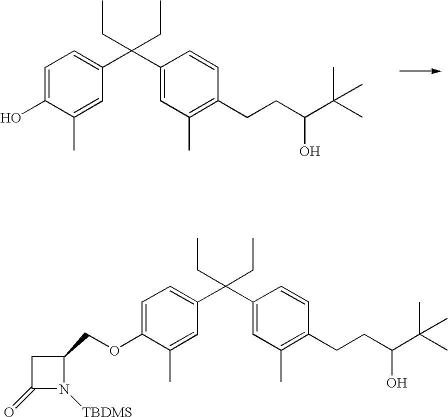

Using the same procedure as described for the preparation of Example 132-(2), the title compound was prepared from 4-{I-Ethyl-1-[4-(3-hydroxy-4,4-dimethyl-pentyl)-3-methyl-phenyl]-propyl}-2-methyl-phenol (compound prepared in Example 18) and (S)-1-(tert-Butyl-dimethyl-silanyl)-4-hydroxymethyl-azetidin-2-one (compound prepared in Example 133-(1)). The yield was 19%.

¹H-NMR (300 MHz, CDCl₃): 0.59 (t, 6H), 0.89 (s, 9H), 1.39 (d, 1H), 1.48-1.57 (m, 1H), 1.57 (s, 15H), 1.74-1.84 (m, 1H), 2.04 (q, 4H), 2.15 (s, 3H), 2.25 (s, 3H), 2.50-2.60 (m, 1H), 2.81-2.91 (m, 2H), 3.11-3.18 (dd, 1H), 3.21-3.27 (dd, 1H), 3.93-3.98 (dd, 1H), 4.02-4.07 (m, 1H), 4.11-4.16 (m, 1H), 6.66 (d, 1H), 6.89-6.97 (m, 4H), 7.01 (d, 1H); MS (ESI+): 602 ([M+Na]⁺).

### (3) Preparation of 2(S)-(4-{i-Ethyl-1-[4-(3-hydroxy-4,4-dimethyl-pentyl)-3-methyl-phenyl]-propyl}-2-methyl-phenoxymethyl)-4-oxo-azetidine-1-carboxylic acid tert-butyl ester

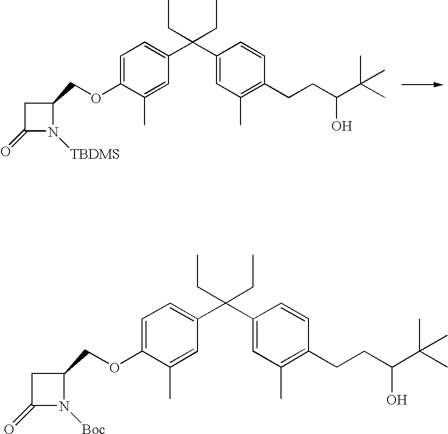

Using the same procedure as described for the preparation of Example 132-(3), the title compound was prepared from 1-(tert-Butyl-dimethyl-silanyl)-4(S)-(4-{1-ethyl-1-[4-(3-hydroxy-4,4-dimethyl-pentyl)-3-methyl-phenyl]-propyl}-2-methyl-phenoxymethyl)-azetidin-2-one (compound prepared in Example 133-(2)). The yield was 67%

¹H-NMR (300 MHz, CDCl₃): 0.59 (t, 6H), 0.89 (s, 9H), 1.47 (s, 9H), 1.48-1.57 (m, 1H), 1.72-1.84 (m, 1H), 2.02 (q, 4H), 2.14 (s, 3H), 2.25 (s, 3H), 2.50-2.60 (m, 1H), 2.81-2.91 (m, 1H), 3.12 (t, 2H), 3.22 (d, 1H), 4.19-4.29 (m, 2H), 4.38 (dd, 1H), 6.68 (d,1H), 6.89-6.94 (m, 4H), 7.01 (d, 1H).

### (4) Preparation of [1 (S)-(4-{1-Ethyl-1-[4-(3-hydroxy4,4-dimethyl-pentyl)-3-methyl-phenyl]-propyl}-2-methyl-phenoxymethyl)-3-hydroxy-propyl]-carbamic acid tert-butyl ester

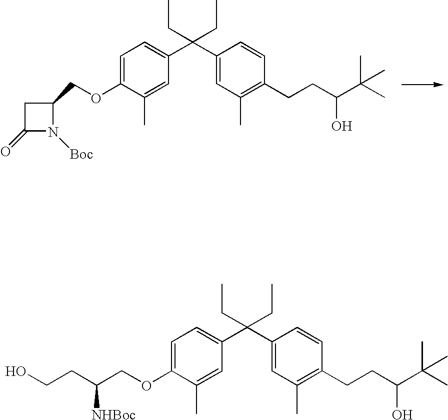

Using the same procedure as described for the preparation of Example 132-(4), the title compound was prepared from 2(S)-(4-{1-Ethyl-1-[4-(3-hydroxy-4,4-dimethyl-pentyl)-3-methyl-phenyl]-propyl}-2-methyl-phenoxymethyl)4-oxo-azetidine-1-carboxylic acid tert-butyl ester (compound prepared in Example 133-(3)). The yield was 84%

¹H-NMR (300 MHz, CDCl₃): 0.59 (t, 6H), 0.89 (s, 9H), 1.45 (s, 9H), 1.48-1.57 (m, 1H), 1.73-1.89 (m, 3H), 2.03 (q, 4H), 2.16 (s, 3H), 2.25 (s, 3H), 2.50-2.60 (m, 1H), 2.81-2.91 (m,1H), 3.22 (d,1H), 3.39 (m,1H), 3.71 (m, 2H), 3.91-3.95 (dd,1H), 4.08-4.20 (m, 2H), 4.38 (dd, 1H), 6.66 (d, 1H), 6.88-6.98 (m, 4H), 7.01 (d, 1H).

### (5) Preparation of 1 (S)-(4-{1-[4-((S)-2-Amino-4-hydroxy-butoxy)-3-methyl-phenyl]-1-ethyl-propyl}-2-methyl-phenyl)-4,4-dimethyl-pentan-3-ol

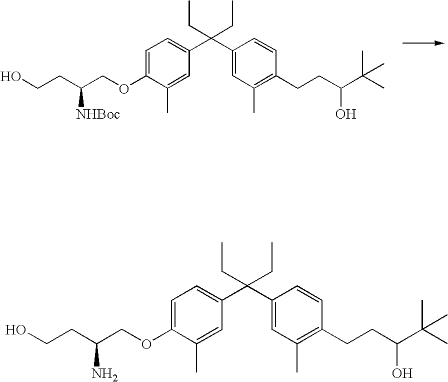

Using the same procedure as described for the preparation of Example 132-(5), the title compound was prepared from [1 (S)-(4-{1-Ethyl-1-[4-(3-hydroxy-4,4-dimethyl-pentyl)-3-methyl-phenyl]-propyl}-2-methyl-phenoxymethyl)-3-hydroxy-propyl]-carbamic acid tert-butyl ester (compound prepared in Example 133-(4)). The yield was 42%

¹H-NMR (300 MHz, CDCl₃): 0.59 (t, 6H),.0.89 (s, 9H), 1.45-1.54 (m,1H), 1.73-1.83 (m, 3H), 2.03 (q, 4H), 2.18 (s, 3H), 2.24 (s, 3H), 2.28-2.37 (m, 2H), 2.50-2.60 (m, 1H), 2.81-2.91 (m,1H), 3.24 (m, 1H), 3.84-4.03 (m, 2H), 4.24-4.29 (m, 1H), 4.45-4.51 (m,1H), 6.69 (d, 1H), 6.88-6.95 (m, 4H), 7.01 (d, 1H); MS (ESI+): 470 ([M+H]⁺).

### Example 134

### Preparation of 4-[2-(4-{(1-Ethyl-1-[4-(3-hydroxy-4,4-dimethyl-pentyl)-3-methyl-phenyl]-propyl}-2-methyl-phenyl)-ethanesulfonyl]-butyric acid

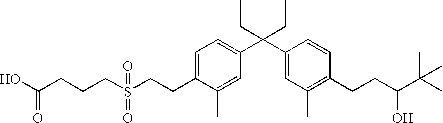

### (1) Preparation of Trifluoro-methanesulfonic acid 4-{1-[4-(4,4-dimethyl-3-oxo-pentyl)-3-methyl-phenyl]-1-ethyl-propyl}-2-methyl-phenyl ester

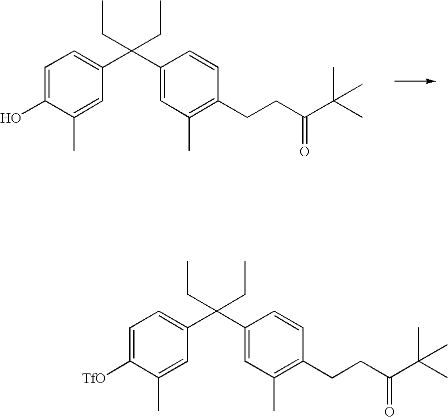

To a solution of 1-{4-[1-Ethyl-1-(4-hydroxy-3-methyl-phenyl)-propyl]-2-methyl-phenyl}-4,4-dimethyl-pentan-3-one (compound prepared in Example 18-(3)) (6.4 g, 16.8 mmol) in CH₂Cl₂ (100 ml) were added Tf₂O (4.2 ml, 25.2 mmol) and pyridine (4.1 ml, 50.4 mmol) at 0 degrees C. under N₂ atmosphere and the mixture was stirred at room temperature for 5 h. The reaction mixture was poured into sat. NaHCO₃ and the products were extracted with CH₂Cl₂. The extracts were washed with brine, dried over MgSO₄, filtered and concentrated under reduced pressure. The obtained residue was chromatographed on silica gel with n-Hexane: EtOAc (50:1) to give the title compound (7.25 g, 84%).

¹H-NMR (300 MHz, CDCl₃): 0.60 (6H, t), 1.08 (9H, s), 2.05 (4H, q), 2.27 (3H, s), 2.31 (3H, s), 2.69-2.76 (2H, m), 2.80-2.88 (2H, m), 6.84-6.89 (2H, m), 6.96-7.10 (4H, m).

### (2) Preparation of 1-{4-[1-(4-Allyl-3-methyl-phenyl)-1-ethyl-propyl]-2-methyl-phenyl}-4,4-dimethyl-pentan-3-one

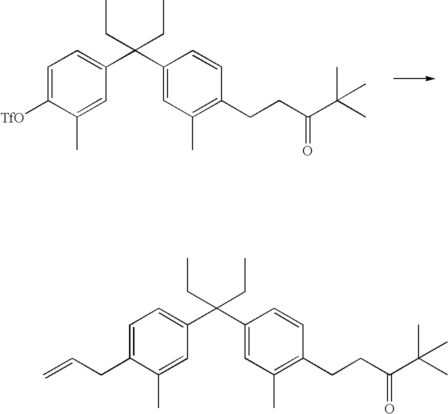

To a solution of Trifluoro-methanesulfonic acid 4-{1-[4-(4,4-dimethyl-3-oxo-pentyl)-3-methyl-phenyl]-1-ethyl-propyl}-2-methyl-phenyl ester (compound prepared in Example 134-(1)) (7.25 g,14.15 mmol) in DMF (70 ml) under argon atmosphere were added allyltributyltin (17.55 ml, 56.59 mmol), PdCl₂(PPh₃)₂ (993 mg, 1.415 mmol), PPh₃ (742 mg, 2.830 mmol) and LiCl (5.04 g,119 mmol) and the mixture was stirred at 140 degrees C. for 3 h. The mixture was quenched with sat. NH₄Cl aq. and filtered through celite. The filtrate was poured into water and the products were extracted with diethyleter. The extracts were washed with brine, dried over MgSO₄, filtered and concentrated under reduced pressure. The obtained residue was chromatographed on silica gel with n-Hexane:EtOAc (50:1) to give the title compound (4.79 g, 84%).

¹H-NMR (300 MHz, CDCl₃): 0.59 (6H, t), 1.10 (9H, s), 2.06 (4H, q), 2.22 (3H, s), 2.26 (3H, s), 2.69-2.78 (2H, m), 2.80-2.87 (2H, m), 3.33 (2H, d), 4.95-5.05 (2H, m), 5.88-6.02 (1H, m), 6.88-7.00 (6H, m).

### (3) Preparation of 1-{4-[1-(4-Allyl-3-methyl-phenyl)-1-ethyl-propyl]-2-methyl-phenyl}-4,4-dimethyl-pentan-3-ol

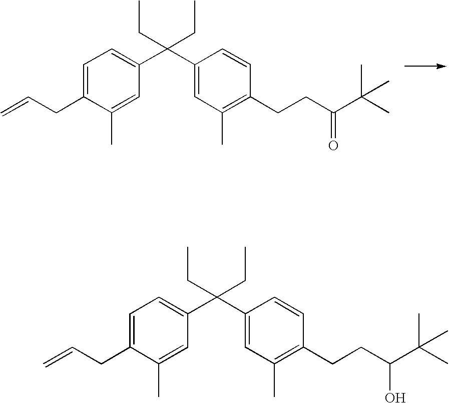

To a solution of 1-{4-[1-(4-Allyl-3-methyl-phenyl)-1-ethyl-propyl]-2-methyl-phenyl}4,4-dimethyl-pentan-3-one (compound prepared in Example 134-(2)) (4.79 g, 11.84 mmol) in EtOH (100 ml) was added NaBH₄ (537 mg, 14.21 mmol) at 0 degrees C. and the mixture was stirred at room tempareture for 3 h. The reaction mixture was quenched with water and poured into sat. NH₄Cl aq and the products were extracted with EtOAc. The extracts were washed with brine, dried over MgSO₄, filtered and concentrated under reduced pressure. The obtained residue was chromatographed on silica gel with n-Hexane:EtOAc (20:1) to give the title compound (2.7 g, 56%).

¹H-NMR (300 MHz, CDCl₃): 0.60 (6H, t), 0.90 (9H, s), 1.45-1.59 (1H, m), 1.74-1.85 (1H, m), 2.07 (4H, q), 2.23 (3H, s), 2.28 (3H, s), 2.51-2.60 (1H, m), 2.80-2.90 (1H, m), 3.22-3.29 (1H, m), 3.32 (2H, d), 4.85-5.08 (2H, m), 5.90-6.02 (1H, m), 6.89-7.03 (6H, m).

### (4) Preparation of [1-(2-{4-[1-(4-Allyl-3-methyl-phenyl)-1-ethyl-propyl]-2-methyl-phenyl}-ethyl)-2,2-dimethyl-propoxy]-triethyl-silane

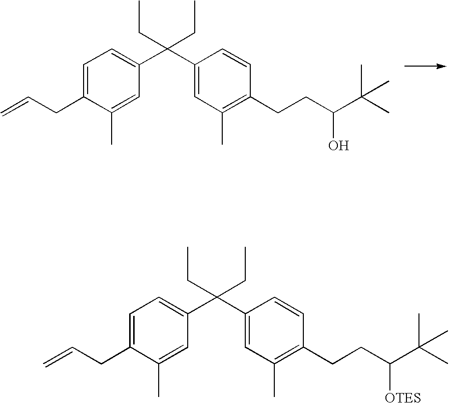

To a stirred solution of 1-{4-[1-(4-Allyl-3-methyl-phenyl)-1-ethyl-propyl]-2-methyl-phenyl}-4,4-dimethyl-pentan-3-ol (compound prepared in Example 134-(3)) (1.40 g, 3.44 mmol) in anhydrous-DMF (11.5 ml) were added Et₃SiCl (0.69 ml, 4.12 mmol) and imidazole (585 mg, 8.59 mmol) at 0 degrees C. under N₂ atmosphere and the mixture was stirred at room temperature for 16 h. The reaction mixture was poured into water and the products were extracted with EtOAc. The extracts were washed with 1 N HCl, sat. NaHCO₃, and brine, dried over Na₂SO₄, filtered and concentrated under reduced pressure. The obtained residue was chromatographed on silica gel with n-Hexane:EtOAc (30:1) to give the title compound (1.79 g, 100%) as colorless sticky oil.

¹H-NMR (300 MHz, CDCl₃): 7.05-6.85 (m, 6H), 6.00-5.90 (m, 1H), 5.10-4.90 (m, 2H), 3.40-3.30 (m, 3H), 2.85-2.70 (m,1H), 2.50-2.40 (m,1H), 2.25 (s, 3H), 2.20 (s, 3H), 2.05 (q, 4H), 1.85-1.70 (m, 1H), 1.55-1.40 (m, 1H), 1.05-0.90 (m, 15H), 0.70-0.50 (m, 15H).

### (5) Preparation of 2-(4-{1-[4-(4,4-Dimethyl-3-triethylsilanyloxy-pentyl)-3-methyl-phenyl]-1-ethyl-propyl}-2-methyl-phenyl)-ethanol

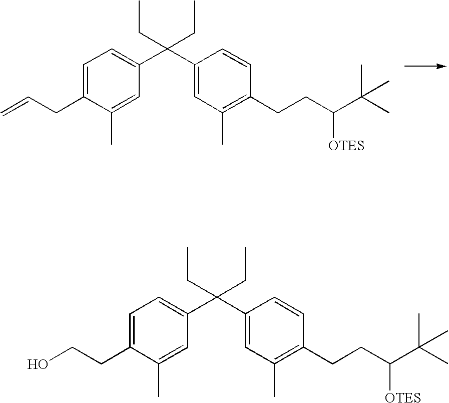

To a stirred solution of [1-(2-{4-[1-(4-Allyl-3-methyl-phenyl)-1-ethyl-propyl]-2-methyl-phenyl}-ethyl)-2,2-dimethyl-propoxy]-triethyl-silane (compound prepared in Example 134-(4)) (1.79 g, 3.44 mmol) in Acetone/tBuOH/Water (4/1/1 v/v, 96 ml) were added NMO (1.01 g, 8.62 mmol) and OsO₄ (2.5% w/w in tBuOH, 4.30 ml, 10 mol %) at room temp. under N₂ atmosphere. After stirring for 12.0 h, the reaction mixture was quenched with sat. NaHCO₃ aq. and stirred for additionally 20 min. The reaction mixture was extracted with EtOAc. Resulted organic layer was washed with brine and dried over anhydrous Na₂SO₄, filtered, and evaporated under reduced pressure. The obtained residue was dissolved in EtOH/Water (1/1 v/v, 82 ml). To the solution, NaIO₄ (866 mg, 4.05 mmol) and NaBH₄ (460 mg, 12.2 mmol) was added and the mixture was stirred at 0 degrees C. for 30 min. The reaction mixture was poured into sat. NH₄Cl aq. and the products were extracted with EtOAc. The extracts were washed with brine, dried over Na₂SO₄, filtered and concentrated under reduced pressure. The obtained residue was chromatographed on silica gel with n-Hexane:EtOAc (10:1) to give the title compound (1.33 g, 74%) as colorless sticky oil.

¹H-NMR (300 MHz, CDCl₃): 7.05-6.85 (m, 6H), 3.90-3.80 (m, 2H), 3.40 (dd,1H) 2.90-2.70 (m, 3H), 2.50-2.40 (m, 1H), 2.30 (s, 3H), 2.25 (s, 3H), 2.05 (q, 4H), 1.85-1.70 (m,1H), 1.55-1.40 (m,1H), 1.00 (t, 9H), 0.90 (t, 9H), 0.60 (t, 6H).

### (6) Preparation of {1-[2-(4.-{1-[4-(2-Bromo-ethyl)-3-methyl-phenyl]-1-ethyl-propyl}-2-methyl-phenyl)-ethyl]-2,2-dimethyl-propoxy}-triethyl-silane

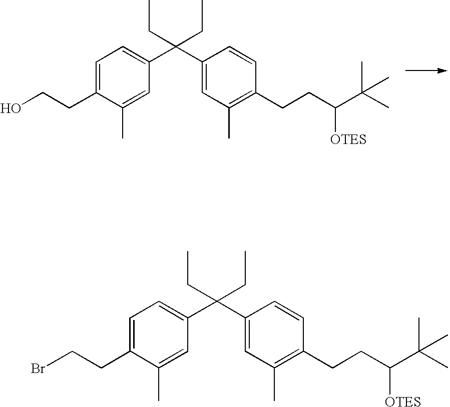

To a stirred solution of 2-(4-{1-[4-(4,4-Dimethyl-3-triethylsilanyloxy-pentyl)-3-methyl-phenyl]-1-ethyl-propyl}-2-methyl-phenyl)-ethanol (compound prepared in Example 134-(5)) (1.33 g, 2.54 mmol) in CH₂Cl₂ (8.5 ml) were added CBr₄ (1.05 g, 3.18 mmol) and PPh₃ (999 mg, 3.80 mmol) at 0 degrees C. under N₂ atmosphere. After stirring for 30 min., the reaction mixture was evaporated under reduced pressure and the obtained residue was purified by silica gel column chromatography (n-Hexane:Acetone=20:1) to give the title compound (829 mg, 54%) as colorless sticky oil.

¹H-NMR (300 MHz, CDCl₃): 7.05-6.85 (m, 6H), 3.50 (t, 3H), 3.40 (dd, 1H), 3.15 (t, 2H), 2.85-2.70 (m,1H), 2.50-2.35 (m,1H), 2.25 (s, 3H), 2.20 (s, 3H), 2.05 (q, 4H), 1.85-1.70 (m, 1H), 1.55-1.40 (m, 1H), 1.00 (t, 9H), 0.90 (t, 9H), 0.60 (t,6H).

### (7) Preparation of 1-(4-{1-[4-(2-Bromo-ethyl)-3-methyl-phenyl]-1-ethyl-propyl}-2-methyl-phenyl-4,4-dimethyl-pentan-3-ol

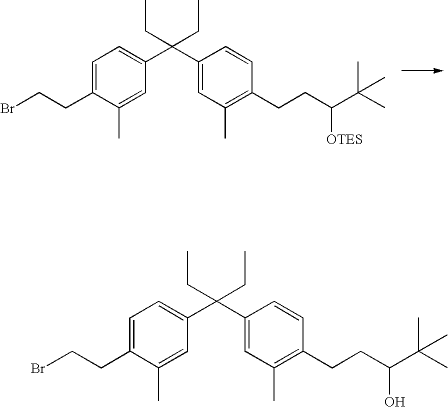

To a stirred solution of {1-[2-(4-{1-[4-(2-Bromo-ethyl)-3-methyl-phenyl]-1-ethyl-propyl}-2-methyl-phenyl)-ethyl]-2,2-dimethyl-propoxy}-triethyl-silane (compound prepared in Example 134-(6)) (1.00 g, 1.70 mmol) in CH₃CN (17 ml)was added 1 N HCl (1.7 ml) at room temperature. After stirring for 3.0 h, the reaction mixture was poured into water and the products were extracted with EtOAc. The extracts were washed with sat. NaHCO₃ aq., dried over Na₂SO₄, filtered and concentrated under reduced pressure. The obtained residue was chromatographed on silica gel with n-Hexane:EtOAc (7:1) to give the title, compound (735 mg, 91%) as colorless sticky oil.

¹H-NMR (300 MHz, CDCl₃): 7.05-6.85 (m, 6H), 3.50 (t, 3H), 3.25 (m, 1H), 3.15 (t, 2H), 2.90-2.80 (m, 1H), 2.60-2.50 (m, 1H), 2.25 (s, 6H), 2.05 (q, 4H), 1.85-1.70 (m, 1H), 1.55-1.40 (m, 1H), 0.90 (t, 9H), 0.60 (t, 6H).

### (8) Preparation of 4-Mercapto-butyric acid methyl ester

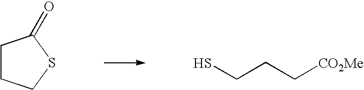

To a stirred solution of Dihydro-thiophen-2-one (2.06 g, 20.2 mmol) in 70.0 mL of MeOH was added Et₃N (4.67 ml, 33.5 mmol) and refluxed for 24 h. The reaction mixture was evaporated under reduced pressure to give the title compound (2.50 g, 92.3%).

¹H-NMR (300 MHz, CDCl₃): 3.70 (s, 3H), 2.60 (m, 2H), 2.48 (t, 2H), 1.95 (m, 2H).

### (9) Preparation of 4-[2-(4-{1-Ethyl-1-[4-(3-hydroxy4,4-dimethyl-pentyl)-3-phenyl]-propyl}-2-methyl-phenyl)-ethylsulfanyl]-butyric acid ethyl ester

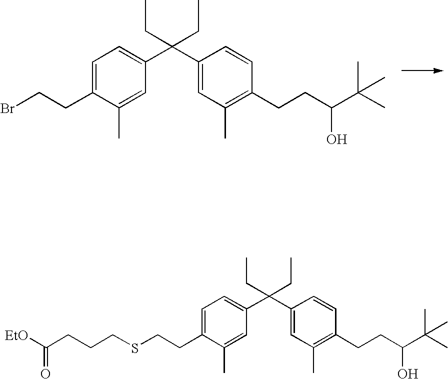

To a stirred solution of 1-(4-{1-[4-(2-Bromo-ethyl)-3-methyl-phenyl]-1-ethyl-propyl}-2-methyl-phenyl)-4,4-dimethyl-pentan-3-ol (compound prepared in Example 134-(7)) (360 mg, 0.76 mmol) and 4-Mercaptb-butyric acid methyl ester (compound prepared in Example 134-(8)) (204 mg, 1.52 mmol) in 3.80 ml of anhydrous DMF was added i-Pr₂EtN (0.20 ml, 1.15 mmol) under N₂ atmosphere at room temperature. The reaction mixture was stirred for 18 h at room temperature. The reaction mixture was concentrated under reduced pressure and the obtained residue was purified by silica gel chromatography (n-Hexane:EtOAc =10:1) to give the title compound (48.5 mg, 12%) as colorless sticky oil.

¹H-NMR (300 MHz, CDCl₃): 7.05-6.85 (m, 6H), 3.70 (s, 3H), 3.25 (d, 1H), 2.90-2.50 (m, 10H), 2.25 (s, 6H), 2.05 (q, 4H), 1.85-1.75 (m,1H), 1.55-1.40 (m,1H), 0.90 (s, 9H), 0.60 (t, 6H).

### (10) Preparation of 4-[2-(4-{1-Ethyl-I -[4-(3-hydroxy-4,4-dimethyl-pentyl)-3-methyl-phenyl]-propyl}-2-methyl-phenyl)-ethanesulfonyl]-butyric acid ethyl ester

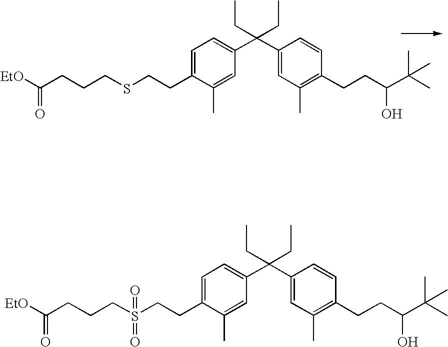

To a stirred solution of 4-[2-(4-{1-Ethyl-1-[4-(3-hydroxy-4,4-dimethyl-pentyl)-3-methyl-phenyl]-propyl}-2-methyl-phenyl)-ethylsulfanyl]-butyric acid ethyl ester (compound prepared in Example 134-(9)) (100 mg, 0.19 mmol) in 2.00 ml of CH₂Cl₂ was added mCPBA (144 mg, 0.48 mmol) at 0 degrees C. and gradually warmed up to room temperature. The reaction mixture was stirred for 3.0 h at room temperature. The reaction mixture was poured into Na₂S₂O₃ aq. and the products were extracted with CH₂Cl₂. The extracts were washed with brine, dried over Na₂SO₄, filtered and concentrated under reduced pressure. The obtained residue was chromatographed on silica gel with n-Hexane:EtOAc (4:1) to give the title compound (103 mg, 99%) as colorless sticky oil.

¹H-NMR (300 MHz, CDCl₃): 7.05-6.85 (m, 6H), 3.70 (s, 3H), 3.30-3.00 (m, 7H), 2.95-2.80 (m, 1H), 2.65-2.50 (m, 3H), 2.30 (s, 3H), 2.25 (s, 3H), 2.20-2.05 (m, 6H), 1.85-1.75 (m, 1H), 1.60-1.45 (m, 1H), 0.90 (s, 9H), 0.60 (t, 6H).

### (11) Preparation of 4-[2-(4-{1-Ethyl-1-[4-(3-hydroxy-4,4-dimethyl-pentyl)-3-methyl-phenyl]-propyl}-2-methyl-phenyl)-ethanesulfonyl]-butyric acid

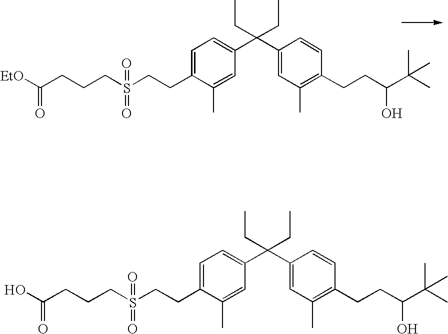

To a stirred solution of 4-[2-(4-{1-Ethyl-1-[4-(3-hydroxy-4,4-dimethyl-pentyl)-3-methyl-phenyl]-propyl}-2-methyl-phenyl)-ethanesulfonyl]-butyric acid ethyl ester (compound prepared in Example 134-(10)) (103 mg, 0.18 mmol) in 2.50 ml of Dioxane/Water (4/1 v/v) was added NaOH (14.4 mg, 0.36 mmol) at room temperature. The reaction mixture was stirred for 2.5 h at room temperature. The reaction mixture was poured into 1 N HCl and extracted with EtOAc. The extracts were washed with brine, dried over Na₂SO₄, filtered and concentrated under reduced pressure. The obtained residue was chromatographed on silica gel with n-Hexane:EtOAc (1:1) to give the title compound (44.1 mg, 45%) as colorless sticky oil.

¹H-NMR (300 MHz, CDCl₃): 7.05-6.85 (m, 6H), 3.30-3.00 (m, 7H), 2.90-2.75 (m, 1H), 2.60-2.40 (m, 3H), 2.20 (s, 6H), 2.20-2.05 (m, 6H), 1.85-1.70 (m, 1H), 1.55-1.40 (m, 1H), 0.90 (s, 9H), 0.60 (t, 6H); MS (ESI−): 543 ([M−H]⁻).

### Example 135

### Preparation of 3-[2-(4-{1-Ethyl-1-[4-(3-hydroxy-4,4-dimethyl-pentyl)-3-methyl-phenyl]-propyl}-2-methyl-phenyl)-ethanesulfonyl]-propionic acid

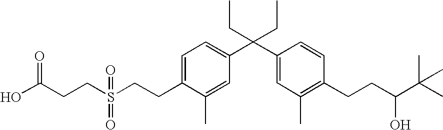

### (1) Preparation of 3-[2-(4-{1-Ethyl-I -[4-(3-hydroxy-4,4-dimethyl-pentyl)-3-methyl-phenyl]-propyl}-2-methyl-phenyl)-ethylsulfanyl]-propionic acid methyl ester

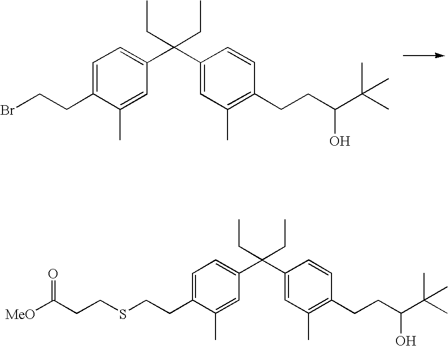

Using the same procedure as described for the preparation of Example 134-(9), the title compound was prepared from 1-(4-{1-[4-(2-Bromo-ethyl)-3-methyl-phenyl]-1-ethyl-propyl}-2-methyl-phenyl)-4,4-dimethyl-pentan-3-ol (compound prepared in Example 134-(7)) and Methyl 3-mercaptopropionate. The yield was 36%.

¹H-NMR (300 MHz, CDCl₃): 7.05-6.85 (m, 6H), 3.70 (s, 3H), 3.25 (d, 1H), 2.90-2.50 (m, 1 OH), 2.25 (s, 6H), 2.05 (q, 4H), 1.85-1.70 (m, 1H), 1.55-1.40 (m, 1H), 0.90 (t, 9H), 0.60 (t, 6H); MS (ESI+) 535 ([M+Na]⁺).

### (2) Preparation of 3-[2-(4-{1-Ethyl-1-[4-(3-hydroxy-4,4-dimethyl-pentyl)-3-methyl-phenyl]-propyl}-2-methyl-phenyl)-ethanesulfonyl]-propionic acid methyl ester

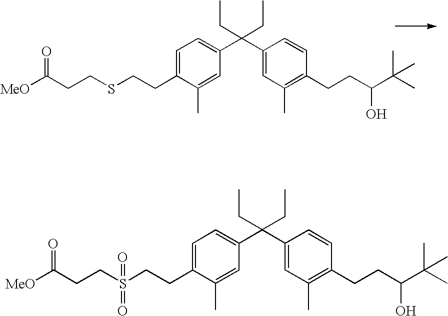

Using the same procedure as described for the preparation of Example 134-(10), the title compound was prepared from 3-[2-(4-{1-Ethyl-1-[4-(3-hydroxy-4,4-dimethyl-pentyl)-3-methyl-phenyl]-propyl}-2-methyl-phenyl)-ethylsulfanyl]-propionic acid methyl ester (compound prepared in Example 135-(1)). The yield was 91%.

¹H-NMR (300 MHz, CDCl₃): 7.05-6.85 (m, 6H), 3.70 (s, 3H), 3.30-3.10 (m, 7H), 2.90-2.75 (m, 3H), 2.65-2.50 (m,1H), 2.30 (s, 3H), 2.25 (s, 3H), 2.05 (q, 4H), 1.85-1.70 (m, 1H), 1.55-1.40 (m, 1H), 0.90 (t, 9H), 0.60 (t, 6H); MS (ESI+): 567 ([M+Na]⁺).

### (3) Preparation of 3-[2-(4-{1-Ethyl-1-[4-(3-hydroxy-4,4-dimethyl-pentyl)-3-methyl-phenyl]-propyl}-2-methyl-phenyl)-ethanesulfonyl]-propionic acid

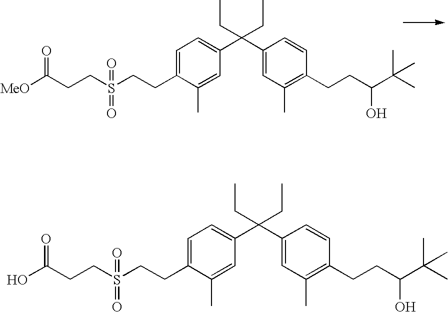

Using the same procedure as described for the preparation of Example 134-(11), the title compound was prepared from 3-[2-(4-{1-Ethyl-1-[4-(3-hydroxy-4,4-dimethyl-pentyl)-3-methyl-phenyl]-propyl}-2-methyl-phenyl)-ethanesulfonyl]-propionic acid methyl ester (compound prepared in Example 135-(2)). The yield was 19%.

¹H-NMR (300 MHz, CDCl₃); 7.05-6.85 (m, 6H), 3.30-3.15 (m, 6H), 3.10-3.00 (m, 2H), 2.90-2.75 (m, 3H), 2.65-2.50 (m, 1H), 2.25 (s, 6H), 2.05 (m, 4H), 1.85-1.70 (m, 1H), 1.55-1.40 (m, 1H), 0.90 (t, 9H), 0.60 (t, 6H); MS (ESI−): 529 ([M−H]⁻).

### Example 136

### Preparation of N-tert-Butyl-2-(4-{1-[4-((S)-2,3-dihydroxy-propoxy)-3-methyl-phenyl]-1-ethyl-propyl}-2-methyl-phenyl)-acetamide

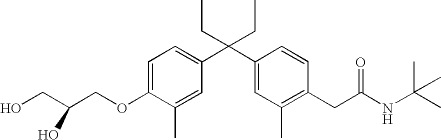

### (1) Preparation of 4-{1-[4-((R)-2,2-Dimethyl-[1,3]dioxolan4-ylmethoxy)-3-methyl-phenyl]-1-ethyl-propyl}-2-methyl-phenol

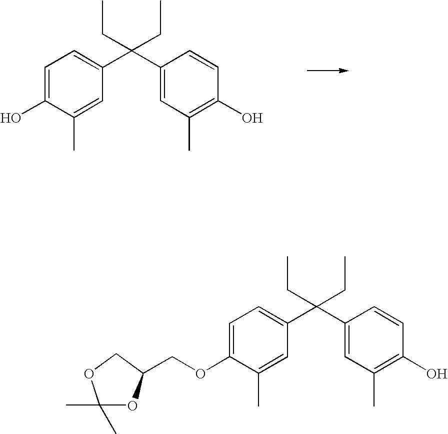

To a solution of 3,3-bis[4-hydroxy-3-methylphenyl]pentane (1 g, 3.52 mmol) in DMSO (9 ml) was added t-BuOK (379 mg, 3.38 mmol) at room temperature under N₂ atmosphere. After 5 min., (S)-(+)-2,2-dimethyl 1,3-dioxolan4-yl-methyl-p-toluenesulfonate (796 mg, 2.78 mmol) was added and the reaction mixture was stirred at 60 degrees C. for 12 h. Then the reaction mixture was poured into sat. NH₄Cl and the products were extracted with EtOAc. The extracts were washed with brine, dried over Na₂SO₄, filtered and concentrated under reduced pressure. The obtained residue was chromatographed on silica gel with n-Hexane:Acetone (20:1) to give the title compound (472 mg, 42.6%) as a white amorphous.

¹H-NMR (300 MHz, CDCl₃): 7.00-6.80 (m, 4H), 6.80-6.60 (m, 2H), 4.90 (s,1H), 4.55-4.45 (m,1H), 4.20-3.90 (m, 4H), 2.10, 2.20(S, 6H), 2.10-1.90 (m, 4H), 1.49 (s, 3H), 1.48 (s, 3H), 0.60 (t, 6H).

### (2) Preparation of Trifluoro-methanesulfonic acid 4-{1-[4-((R)-2,2-dimethyl-[1,3]dioxolan4-ylmethoxy)-3-methyl-phenyl]-1-ethyl-propyl}-2-methyl-phenyl ester

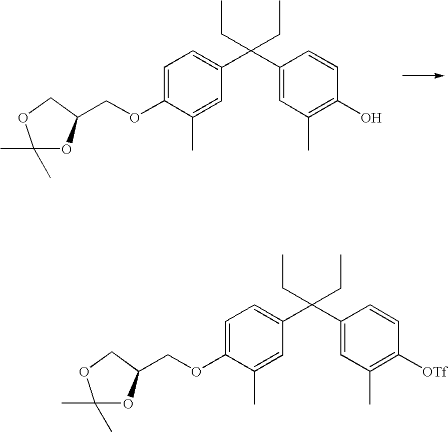

To a stirred solution of 4-{1-[4-((R)-2,2-Dimethyl-[1,3]dioxolan4-ylmethoxy)-3-methyl-phenyl]-1-ethyl-propyl}-2-methyl-phenol (compound prepared in Example 136-(1)) (18.6 g, 46.7 mmol) in CH₂Cl₂ (460 ml) were added Tf₂O (11.8 ml, 70.1 mmol) and pyridine (7.6 ml, 93.4 mmol) and the mixture was stirred at 0 degrees C. under N₂ atmosphere for 1 h. Then the reaction mixture was poured into sat. NaHCO₃ and the products were extracted with CH₂Cl₂. The extracts were washed with brine, dried over Na₂SO₄, filtered and concentrated under reduced pressure. The obtained residue was chromatographed on silica gel with n-Hexane:Acetone (10:1) to give the title compound (14.5 g, 85%) as yellow oil.

¹H-NMR (300 MHz, CDCl₃): 7.00-7.15 (m, 3H), 6.80-7.00 (m, 2H), 6.70 (d, 1H), 4.45-4.55 (m, 1H), 4.15 (m, 1H), 4.05 (m, 1H), 3.904.00 (m, 1H), 2.35 (s, 3H), 2.15 (s, 3H), 2.05 (q, 4H), 1.50 (s, 3H), 1.40 (s, 3H), 0.60 (t, 6H).

### (3) Preparation of (R)4-{4-[1-(4-Allyl-3-methyl-phenyl)-1-ethyl-propyl]-2-methyl-phenoxymethyl}-2,2-dimethyl-[1,3]dioxolane

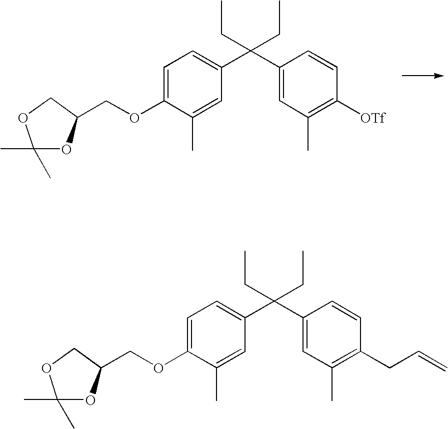

To a stirred solution of Trifluoro-methanesulfonic acid 4-{1-[4-((R)-2,2-dimethyl-[1,3]dioxolan4-ylmethoxy)-3-methyl-phenyl]-1-ethyl-propyl}-2-methyl-phenyl ester (compound prepared in Example 136-(2)) (8.52 g, 16.1 mmol) in DMF (80 ml) were added Pd₂Cl₂(PPh₃)₂ (1.13 g, 10 mol%), PPh₃ (842 mg, 20 mol %) and LiCl (5.72 g, 135 mmol) at room temperature under N₂ atmosphere. After stirred for 10 min., allyltributyltin (19.9 ml, 64.2 mmol) was added to the reaction mixture and stirred at 130 degrees C. for 6 h. The reaction mixture was cooled to room temperature, poured into 1 N HCl and the products were extracted with diethylether. The extracts were washed with brine, dried over Na₂SO₄, filtered and concentrated under reduced pressure. The obtained residue was chromatographed on silica gel with n-Hexane:EtOAc (20:1) to give the title compound (6.08 g, 90%) as colorless oil.

¹H-NMR (300 MHz, CDCl₃): 7.05-6.90 (m, 5H), 6.70 (d, 1H), 6.05-5.85 (m, 1H), 5.05-4.90 (m, 2H), 4.50 (m, 1H), 4.15 (m, 1H), 4.05 (m, 1H), 4.00-3.85 (m, 2H), 3.35 (d, 2H), 2.20 (s, 3H), 2.15 (s, 3H), 2.05 (q, 4H), 1.50 (s, 3H), 1.40 (s, 3H), 0.60 (t, 6H).

### (4) Preparation of 2-(4-{1-[4-((R)-2,2-Dimethyl-[1,3]dioxolan4-ylmethoxy)-3-methyl-phenyl]-1-ethyl-propyl}-2-methyl-phenyl)-ethanol

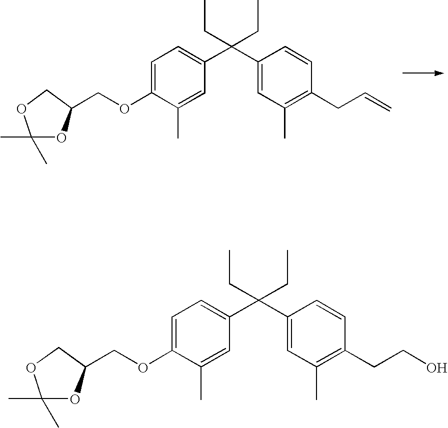

To a stirred solution of (R)4-{4-[1-(4-Allyl-3-methyl-phenyl)-1-ethyl-propyl]-2-methyl-phenoxymethyl}-2,2-dimethyl-[1,3]dioxolane (compound prepared in Example 136-(3)) (5.14 g, 12.2 mmol) in 336 mi of Acetone/t-BuOH/Water (4/1/1 v/v) were added NMO (3.57 g, 30.5 mmol) and OsO₀₄ (2.5% w/w in t-BuOH, 15.2 ml, 10 mol %) at room temperature under N₂ atmosphere and the mixture was stirred for 12 h. The mixture was poured into sat. NaHCO₃ and the products were extracted with EtOAc. The extracts were washed with brine, dried over Na₂SO₄, filtered and concentrated under reduced pressure to give 6.18 g of crude diol compound. The obtained crude diol compound (6.18 g) was dissolved in 274 ml of EtOH/Water (1/1 v/v). Then NaIO₄ (2.89 g, 13.5 mmol) was added to the reaction mixture at 0 degrees C. and followed the addition of NaBH₄ (1.54 g, 13.5 mmol) portion wise. The reaction mixture was stirred for 30 min and poured into sat. NH₄Cl and the products were extracted with EtOAc. The extracts were washed with brine, dried over Na₂SO₄, filtered and concentrated under reduced pressure. The obtained residue was chromatographed on silica gel with n-Hexane:EtOAc (5:1) to give the title compound (3.46 g, 67%) as yellowish oil.

¹H-NMR (300 MHz, CDCl₃): 7.05-6.90 (m, 5H), 6.70 (d, 1H), 4.45 (m, 1H), 4.15 (m, 1H), 4.05 (m, 1H), 4.00-3.85 (m, 2H), 3.70 (t, 2H), 2.85 (t, 2H), 2.30 (s, 3H) 2.15 (s, 3H), 2.05 (q, 4H), 1.50 (s, 3H), 1.40 (s, 3H), 0.60 (t, 6H).

### (5) Preparation of (4-{1-[4-((R)-2,2-Dimethyl-[1,3]dioxolan-4-ylmethoxy)-3-methyl-phenyl]-1-ethyl-propyl}-2-methyl-phenyl)-acetic acid

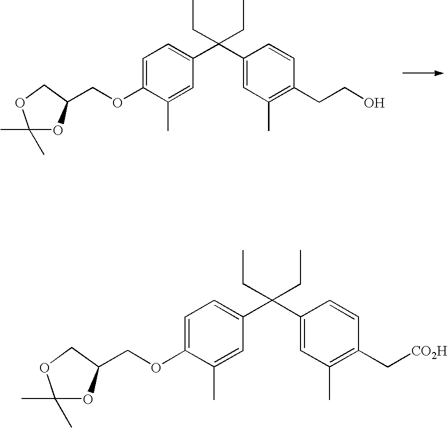

To a stirred solution of 2-(4-{1-[4-((R)-2,2-Dimethyl-[1,3]dioxolan4-ylmethoxy)-3-methyl-phenyl]-1-ethyl-propyl}-2-methyl-phenyl)-ethanol (compound prepared in Example 136-(4)) (1.34 g, 3.14 mmol) in CH₃CN (15 ml) and NaH₂PO₄ buffer solution (0.67M, 11.4 ml) were added TEMPO (34.3 mg, 0.22 mmol), NaOCl₂ (710 mg, 6.28 mmol) in water (3 ml) and NaOCl (0.00387 ml, 2 mol %). The reaction mixture was stirred for 4.0 h at 35 degrees C. The mixture was poured into sat. NaHCO₃ and the products were extracted with EtOAc. The extracts were washed with brine, dried over Na₂SO₄, filtered and concentrated under reduced pressure. The obtained residue was chromatographed on silica gel with n-Hexane:EtOAc (5:1) to give the title compound (1.21 g, 95%) as colorless sticky oil.

¹H-NMR (300 MHz, CDCl₃): 7.05 (m, 1H), 7.00-6.90 (m, 4H), 6.70 (d, 1H), 4.45 (m,1H), 4.15 (m,1H), 4.05 (m,1H), 4.00-3.90 (m, 2H), 3.60 (s, 2H), 2.25 (s, 3H), 2.15 (s, 3H), 2.05 (q, 4H), 1.45 (s, 3H), 1.40 (s, 3H), 0.60 (t, 6H).

### (6) Preparation of N-tert-Butyl-2-(4-{1-[4-((R)-2,2-dimethyl-[1,3]dioxolan-4-ylmethoxy)-3-methyl-phenyl]-1-ethyl-propyl}-2-methyl-phenyl)-acetamide

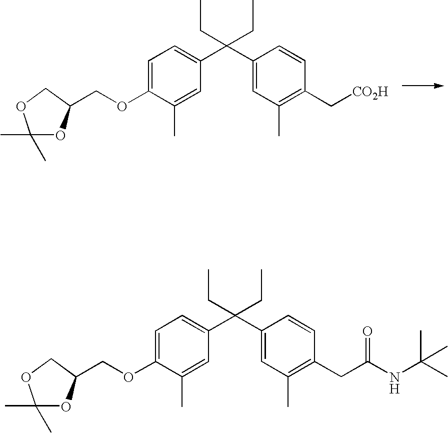

To a stirred solution of (4-{1-[4-((R)-2,2-Dimethyl-[1,3]dioxolan-4-ylmethoxy)-3-methyl-phenyl]-1-ethyl-propyl}-2-methyl-phenyl)-acetic acid (compound prepared in Example 136-(5)) (530 mg, 1.20 mmol) in anhydrous DMF (9.3 ml) were added t-BuNH₂ (263 mg, 3.60 mmol), EDC (345 mg, 1.80 mmol), HOBT (243 mg, 1.80 mmol) and i-Pr₂EtN (0.31 ml, 3.60 mmol) at 0 degrees C. and the mixture was stirred at room temperature overnight. The reaction mixture was poured into water and the products were extracted with EtOAc. The extracts were washed with 1 N HCl, sat. NaHCO₃ aq. and brine, dried over Na₂SO₄, filtered and concentrated under reduced pressure. The obtained residue was chromatographed on silica gel with n-Hexane:Acetone (4:1) to give the title compound (525 mg, 88%) as a white amorphous.

¹H-NMR (300 MHz, CDCl₃): 7.05-6.90 (m, 4H), 6.85 (m,1H), 6.70 (d,1H), 5.05 (bs,1H), 4.50 (m,1H), 4.15 (m,1H), 4.05 (m, 1H), 4.00-3.90 (m, 2H), 3.50 (s, 2H), 2.25 (s, 3H), 2.15 (s, 3H), 2.05 (q, 4H), 1.45 (s, 3H), 1.40 (s, 3H), 1.25 (s, 9H), 0.60 (t, 6H); MS (ESI+): 496 ([M+H]⁺).

### (7) Preparation of N-tert-Butyl-2-(4-{1-[4-((S)-2,3-dihydroxy-propoxy)-3-methyl-phenyl]-1-ethyl-propyl}-2-methyl-phenyl)-acetamide

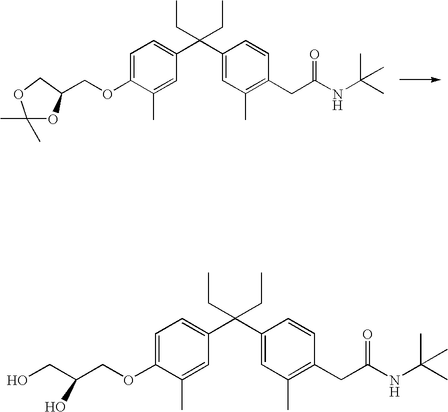

To a stirred solution of N-tert-Butyl-2-(4-{1-[4-((R)-2,2-dimethyl-[1,3]dioxolan-4-ylmethoxy)-3-methyl-phenyl]-1-ethyl-propyl}-2-methyl-phenyl)-acetamide (compound prepared in Example 136-(6)) (525 mg, 1.06 mmol) in THF/H₂O (5/1 v/v, 4.9 ml) was added TFA (0.82 ml, 10.6 mmmol) at 0 degrees C. under N₂ atmosphere and the mixture was stirred at room temperature for 5 h. The mixture was poured into sat. NaHCO₃ aq. and the products were extracted with CH₂Cl₂. The extracts were washed with brine, dried over Na₂SO₄, filtered and concentrated under reduced pressure. The obtained residue was chromatographed on silica gel with n-Hexane:EtOAc (1:1) to give the title compound (233 mg, 48%) as colorless sticky oil.

¹H-NMR (300 MHz, CDCl₃): 7.05-6.90 (m, 4H), 6.85 (m, 1H), 6.70 (d, 1H), 5.05 (bs, 1H), 4.15 (m, 1H), 4.05 (m, 2H), 4.00-3.90 (m, 2H), 3.50 (s, 2H), 2.60 (d, 1H), 2.25 (s, 3H), 2.15 (s, 3H), 2.05 (m, 5H), 1.25 (s, 9H), 0.60 (t, 6H); MS (ESI+): 456 ([M+H]⁺).

### Example 137

### Preparation of (4-{1-[4-((S)-2,3-Dihydroxy-propoxy)-3-methyl-phenyl]-1-ethyl-propyl}-2-methyl-phenyl)-acetic acid tert-butyl ester

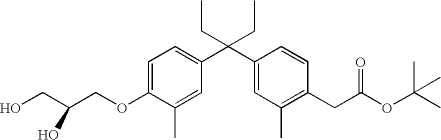

### (1) Preparation of (4-{1-[4-((R)-2,2-Dimethyl-[1,3]dioxolan-4-ylmethoxy)-3 -methyl-phenyl]-1-ethyl-propyl}-2-methyl-phenyl)-acetic acid tert-butyl ester

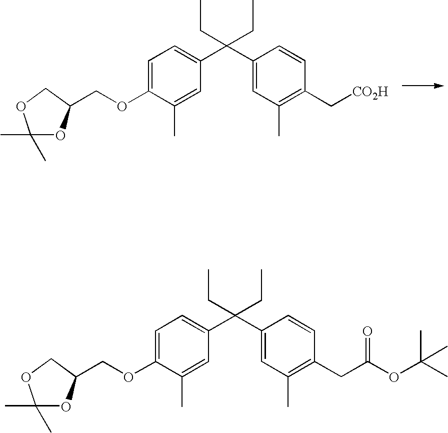

To a stirred solution of (4-{1-[4-((R)-2,2-Dimethyl-[1,3]dioxolan-4-ylmethoxy)-3-methyl-phenyl]-1-ethyl-propyl}-2-methyl-phenyl)-acetic acid (compound prepared in Example 136-(5)) (408 mg, 0.93 mmol) in CH₂Cl₂ (9,3 ml) were added t-BuOH (207 mg, 2.79 mmol), DCC (230 mg, 1.11 mmol) and DMAP (11.4 mg, 10 mol %) at 0 degrees C. and the mixture was stirred at room temperature overnight. The reaction mixture was poured into water and the products were extracted with EtOAc. The extracts were washed with 1 N HCl, sat. NaHCO₃ aq. and brine, dried over Na₂SO₄, filtered and concentrated under reduced pressure. The obtained residue was chromatographed on silica gel with n-Hexane:Acetone (5:1) to give the title compound (217 mg, 47%) as a white amorphous.

¹H-NMR(300 MHz, CDCl₃): 7.05-6.85 (m, 6H), 6.65 (d,1H), 4.50 (m,1H), 4.20 (m, 1H), 4.05 (m, 1H), 4.00-3.90 (m, 2H), 3.50 (s, 2H), 2.25-(s, 3H), 2.15 (s, 3H), 2.05 (q, 4H), 1.45 (s, 3H), 1.40 (s, 12H), 0.60 (t, 6H); MS (ESI+): 514 ([M+NH₄]⁺).

### (2) Preparation of (4-{1-[4-((S)-2,3-Dihydroxy-propoxy)-3-methyl-phenyl]-1-ethyl-propyl}-2-methyl-phenyl)-acetic acid tert-butyl ester

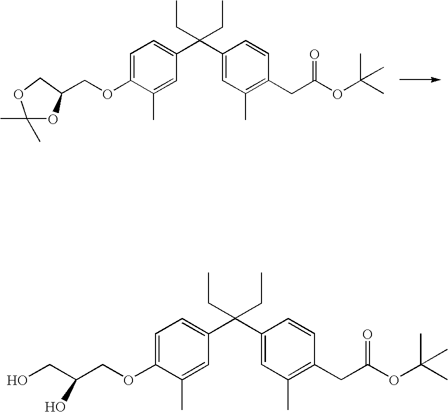

Using the same procedure as described for the preparation of Example 136-(7), the title compound was prepared from (4-{1-[4-((R)-2,2-Dimethyl-[1,3]dioxolan-4-ylmethoxy)-3-methyl-phenyl]-1-ethyl-propyl}-2-methyl-phenyl)-acetic acid tert-butyl ester (compound prepared in Example 137-(1)). The yield was 75%.

¹H-NMR (300 MHz, CDCl₃): 7.05 (m, 1H), 7.00-6.90 (m, 4H), 6.65 (d, 1H), 4.50 (m, 1H), 4.15 (m, 1H), 4.05 (m, 1H), 3.90-3.70 (m, 2H), 3.50 (s, 2H), 2.75 (d, 1H), 2.25 (s, 3H), 2.15 (s,.3H), 2.05 (m, 5H), 1.40 (s, 9H), 0.60 (t, 6H); MS (ESI+): 474 ([M+NH₄]⁺).

### Example 138

### Preparation of 3-[4-(1-Ethyl-1-{3-methyl-4-[2-(2-methyl-propane-2-sulfinyl)-ethyl]-phenyl}-propyl)-2-methyl-phenoxy]-propane-1,2(S)-diol

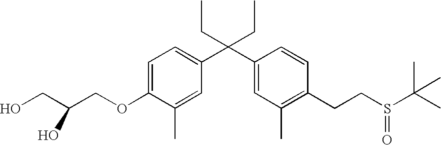

### (1) Preparation of (R)-4-(4-{1-[4-(2-Bromo-ethyl)-3-methyl-phenyl]-1-ethyl-propyl}-2-methyl-phenoxymethyl)-2,2-dimethyl-[1,3]dioxolane

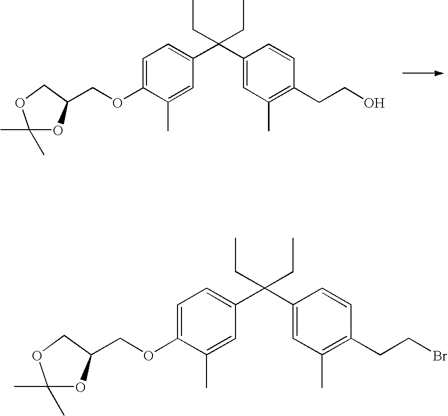

To a stirred solution of 2-(4-{1-[4-((R)-2,2-Dimethyl-[1,3]dioxolan-4-ylmethoxy)-3-methyl-phenyl]-1-ethyl-propyl}-2-methyl-phenyl)-ethanol (compound prepared in Example 136-(4)) (2.51 g, 5.88 mmol) in 20 ml of CH₂Cl₂ were added CBr₄ (2.44 g, 7.36 mmol) and PPh₃ (2.31 g, 8.81 mmol) at 0 degrees C. under N₂ atmosphere. After stirred for 30 min., the reaction mixture was evaporated under reduced pressure and the obtained residue was purified by silica gel chromatography (n-Hexane:Acetone=10:1) to give the title compound (2.76 g, 96%) as yellowish oil.

¹H-NMR (300 MHz, CDCl₃): 7.05-6.90 (m, 5H), 6.70 (d, 1H), 4.45 (m, 1H), 4.15 (m,1H), 4.05 (m,1H), 4.00-3.85 (m, 2H), 3.50 (t, 2H), 3.10 (t, 2H), 2.25 (s, 3H), 2.15 (s, 3H), 2.05 (q, 4H), 1.48 (s, 3H), 1.40 (s, 3H), 0.60 (t, 6H).

### (2) Preparation of (R)-4-(4-{1-[4-(2-tert-Butylsulfanyl-ethyl)-3-methyl-phenyl]-1-ethyl-propyl}-2-methyl-phenoxymethyl)-2,2-dimethyl-[1,3]dioxolane

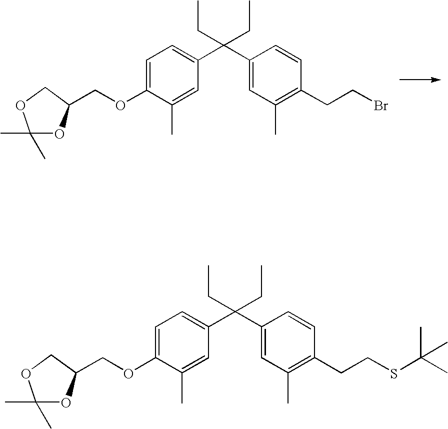

To a stirred solution of (R)-4-(4-{1-[4-(2-Bromo-ethyl)-3-methyl-phenyl]-1-ethyl-propyl}-2-methyl-phenoxymethyl)-2,2-dimethyl-[1,3]dioxolane (compound prepared in Example 138-(1)) (1.33 g, 2.72 mmol) in 27.0 ml of acetone were added t-BuSH (0.46 ml, 4.08 mmol) and KOH (275 mg, 4.90 mmol) in 27.0 ml of EtOH. The reaction mixture was refluxed for 5.0 h. The mixture was poured into water and the products were extracted with EtOAc. The extracts were washed with brine, dried over Na₂SO₄, filtered and concentrated under reduced pressure. The obtained residue was chromatographed on silica gel with n-Hexane:EtOAc (10:1) to give the title compound (962 mg, 71%) as yellowish sticky oil.

¹H-NMR (300 MHz, CDCl₃): 7.05 (m,1H), 7.00-6.85 (m, 4H), 6.70 (d,1H), 4.50 (m, 1H), 4.15 (m, 1H), 4.05 (m, 1H), 4.00-3.90 (m, 2H), 2.90-2.65 (m, 4H), 2.25 (s, 3H), 2.15 (s, 3H), 2.05 (q, 4H), 1.45 (s, 3H), 1.40 (s, 3H), 1.35 (s, 9H), 0.60 (t, 6H).

### (3) Preparation of 4(R)[4-(1-Ethyl-1-{3-methyl-4-[2-(2-methyl-propane-2-sulfinyl)-ethyl]-phenyl}-propyl)-2-methyl-phenoxymethyl]-2,2-dimethyl-[1,3]dioxolane

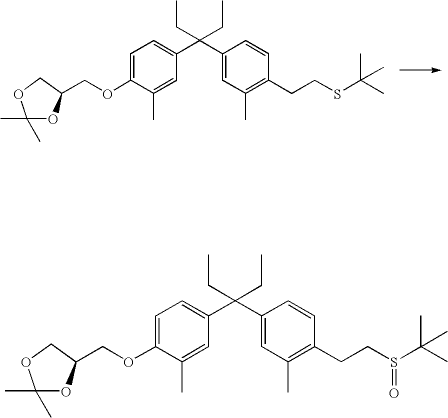

To a solution of (R)-4-(4-{1-[4-(2-tert-Butylsulfanyl-ethyl)-3-methyl-phenyl]-1-ethyl-propyl}-2-methyl-phenoxymethyl)-2,2-dimethyl-[1,3]dioxolane (compound prepared in Example 138-(2)) (379 mg, 0.76 mmol) in EtOH/H₂O (1/2 v/v, 0.76 ml) at 0 degrees C. was added NaIO₄ (163 mg, 0.76 mmol) and the mixture was stirred for 5 h. The mixture was poured into water and the products were extracted with CH₂Cl₂. The extracts were washed with brine, dried over Na₂SO₄, filtered and concentrated under reduced pressure. The obtained residue was chromatographed on silica gel with CH₂Cl₂:MeOH (30:1) to give the title compound (366 mg, 94%) as a white amorphous.

¹H-NMR (300 MHz, CDCl₃): 7.05 (m, 1H), 7.00-6.85 (m, 4H), 6.70 (d, 1H), 4.45 (m, 1H), 4.15 (m, 1H), 4.05 (m, 1H), 4.00-3.90 (m, 2H), 3.20 (m, 1H), 3.00 (m, 1H), 2.70 (m, 2H), 2.30 (s, 3H), 2.15 (s, 3H), 2.05 (q, 4H), 1.45 (s, 3H), 1.40 (s, 3H), 1.25 (s, 9H), 0.60 (t, 6H) M.S.(ESI⁺): [M+H]⁺=515

### (4) Preparation of 3-[4-(1-Ethyl-1-{3-methyl-4-[2-(2-methyl-propane-2-sulfinyl)-ethyl]-phenyl}-propyl)-2-methyl-phenoxy]-propane-1,2(S)-diol

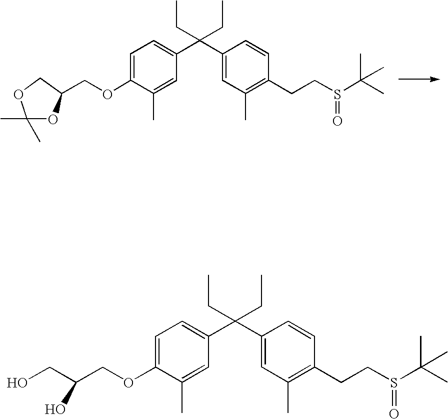

To a stirred solution of 4(R)-[4-(1-Ethyl-1-{3-methyl-4-[2-(2-methyl-propane-2-sulfinyl)-ethyl]-phenyl}-propyl)-2-methyl-phenoxymethyl]-2,2-dimethyl-[1,3]dioxolane (compound prepared in Example 138-(3)) (366 mg, 0.71 mmol) in THF/H₂O (5/1 v/v, 3.2 ml) was added TFA (0.55 ml, 7.10 mmmol) at 0 degrees C. under N₂ atmosphere and the reaction mixture was stirred at room temperature for 5.0 h. The mixture was poured into sat. NaHCO₃ aq. and the products were extracted with CH₂Cl₂. The extracts were washed with brine, dried over Na₂SO₄, filtered and concentrated under reduced pressure. The obtained residue was chromatographed on silica gel with n-Hexane:EtOAc (1:1) to give the title compound (109 mg, 32%) as colorless sticky oil.

¹H-NMR (300 MHz, CDCl₃): 7.05(m., 1H), 7.00-6.85 (m, 4H), 6.70 (d, 1H), 4.15 (m, 1H), 4.05 (m, 2H), 3.90-3.70 (m, 2H), 3.20 (m, 1H), 3.00 (m, 1H), 2.65 (m, 2H), 2.30 (s, 3H), 2.20 (s, 3H), 2.05 (q, 4H), 1.25 (s, 9H), 0.60 (t, 6H); MS (ESI+): 475 ([M+H]⁺).

### Example 139

### Preparation of 3-[4-(1-Ethyl-1-{3-methyl4-[2-(2-methyl-propane-2-sulfonyl)-ethyl]-phenyl}-propyl)-2-methyl-phenoxy]-propane-1,2(S)-diol

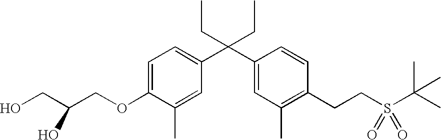

### (1) Preparation of (R)4-[4-(1-Ethyl-1-{3-methyl-4-[2-(2-methyl-propane-2-sulfonyl)-ethyl]-phenyl}-propyl)-2-methyl-phenoxymethyl]-2,2-dimethyl-[1,3]dioxolane

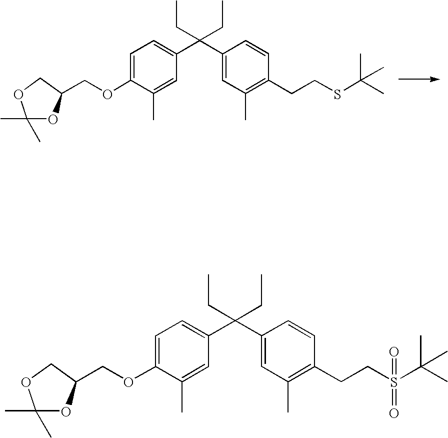

To a stirred solution of (R)-4-(4-{1-[4-(2-tert-Butylsulfanyl-ethyl)-3-methyl-phenyl]-1-ethyl-propyl}-2-methyl-phenoxymethyl)-2,2-dimethyl-[1,3]dioxolane (compound prepared in Example 138-(2)) (503 mg, 1.00 mmol) in CH₂Cl₂ (5 ml) was added mCPBA (757 mg, 2.5 mmol) at 0 degrees C. and the reaction mixture was stirred for 12 h at room temperature. The mixture was poured into Na₂S₂O₃ aq. and the products were extracted with CH₂Cl₂. The extracts were washed with brine, dried over Na₂SO₄, filtered and concentrated under reduced pressure. The obtained residue was chromatographed on silica gel with n-Hexane:EtOAc (3:1) to give the title compound (320 mg, 60%) as a white amorphous.

¹H-NMR (300 MHz, CDCl₃): 7.05 (m, 1H), 7.00-6.85 (m, 4H), 6.70 (d, 1H), 4.45 (m, 1H), 4.15 (m, 1H), 4.05 (m, 1H), 4.00-3.90 (m, 2H), 3.20-3.00 (m, 4H), 2.30 (s, 3H), 2.15 (s, 3H), 2.05 (q, 4H), 1.50 (s, 3H), 1.48 (s, 9H), 1.40 (s, 3H), 0.60 (t, 6H); MS (ESI+): 548 ([M+NH₄]⁺).

### (2) Preparation of (S)-3-[4-(1-Ethyl-1-{3-methyl-4-[2-(2-methyl-propane-2-sulfonyl)-ethyl]-phenyl}-propyl)-2-methyl-phenoxy]-propane-1,2-diol

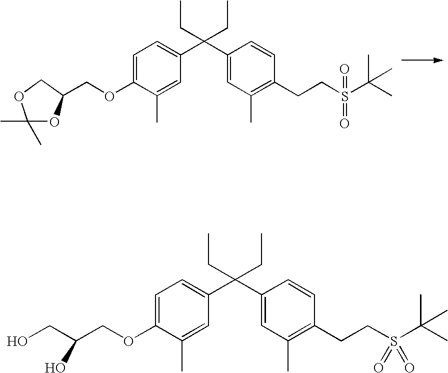

Using the same procedure as described for the preparation of Example 138-(4), the title compound was prepared from (R)-4-[4-(1-Ethyl-1-{3-methyl-4-[2-(2-methyl-propane-2-sulfonyl)-ethyl]-phenyl}-propyl)-2-methyl-phenoxymethyl]-2,2-dimethyl-[1,3]dioxolane (compound prepared in Example 139-(1)). The yield was 56%.

¹H-NMR (300 MHz, CDCl₃): 7.05 (m, 1H), 7.00-6.85 (m, 4H), 6.70 (d, 1H), 4.15 (m,1H), 4.05 (m, 2H), 3.90-3.70 (m, 2H), 3.20-3.00 (m, 4H), 2.55 (d,1H), 2.30 (s, 3H), 2.20 (s, 3H), 2.05 (m, 5H), 1.50 (s, 9H), 0.60 (t, 6H); MS (ESI+): 508 ([M+NH₄]⁺).

### Example 140

### Preparation of 5-(4-{1-Ethyl-1-[4-(3-hydroxy-4,4-dimethyl-pentyl)-3-methyl-phenyl]-propyl}-2-methyl-phenoxy)4(R)-hydroxy-pentanoic acid sodium salt

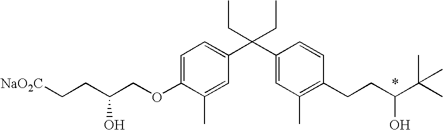

### (1) Preparation of 5(R)-(4-{1-Ethyl-1-[4-(3-hydroxy-4,4-dimethyl-pentyl)-3-methyl-phenyl]-propyl}-2-methyl-phenoxymethyl)-dihydro-furan-2-one

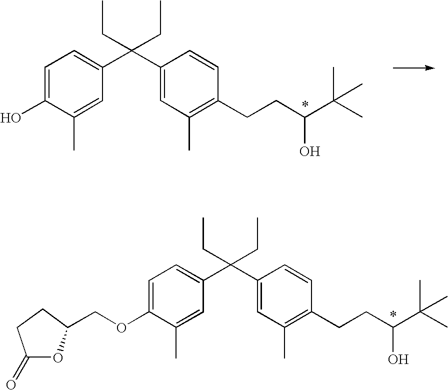

To a stirred solution of 4-{1-Ethyl-1-[4-(3-hydroxy-4,4-dimethyl-pentyl)-3-methyl-phenyl]-propyl}-2-methyl-phenol (compound prepared in Example 19-(2)) (702 mg, 1.83 mmol) in N,N-dimethylacetamide (18 ml) were added K₂CO₃ (633 mg, 4.58 mmol) and toluene-4-sulfonic acid (R)-5-oxo-tetrahydro-furan-2-ylmethyl ester (743 mg, 2.75 mmol) and the mixture was stirred at 130 degrees C. for 3 h. The reaction mixture was cooled to room temperature and poured into aqueous NH₄Cl. The products were extracted with ethyl acetate and hexane. The extracts were washed with brine, dried over MgSO₄, filtered and concentrated in vacuo. The obtained residue was chromatographed on silica gel (ethyl acetate/hexane=20/80 to 35/65) to give the title compound (755 mg, 86%).

¹H-NMR: 0.59 (t, 6H), 0.89 (s, 9H), 1.38 (d, 1H), 1.44-1.55 (m, 1H), 1.74-1.84 (m, 1H), 2.03 (q, 4H), 2.15 (s, 3H), 2.25 (s, 3H), 2.27-2.62 (m, 4H), 2.72-2.91 (m, 2H), 3.21-3.27 (m,1H), 4.044.19 (m, 2H), 4.84-4.91 (m,1H), 6.66 (d, 1H), 6.89-6.97 (m, 4H), 7.02 (d, 1H).

### (2) 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt

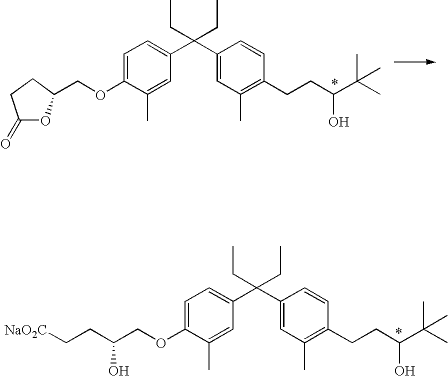

To a solution of 5-(4-{1-Ethyl-1-[4-(3-hydroxy-4,4-dimethyl-pentyl)-3-methyl-phenyl]-propyl}-2-methyl-phenoxymethyl)-dihydro-furan-2-one (compound prepared in Example 140-(1)) (547 mg, 1.14 mmol) in methanol (4.5 ml) and tetrahydrofuran (4.5 ml) was added 1 N KOH solution (4.55 ml) and the mixture was stirred for 1 h at room temperature. The reaction mixture was concentrated in vacuo and poured into aqueous KHSO₄ and the products were extracted with AcOEt. The extracts were washed with H₂O, dried over MgSO₄, filtered and concentrated in vacuo. The obtained residue (524 mg) was dissolved with MeOH (2 ml) and 1.0M solution of NaOMe in MeOH (1.027 ml, 1.051 mmol) was added. The mixture was stirred for 2 min. and concentrated in vacuo to give the title compound (545 mg, 92%).

¹H-NMR (CD₃OD): 0.58 (t, 6H), 0.86 (s, 9H), 1.43-1.54 (m, 1H), 1.73-1.99 (m, 3H), 2.04 (q, 4H), 2.14 (s, 3H), 2.23 (s, 3H), 2.36 (dt, 2H), 2.48-2.58 (m, 1H), 2.81-2.91 (m, 1H), 3.15 (dd, 1H), 3.88-3.97 (m, 3H), 6.75 (d, 1H), 6.83-7.02 (m, 5H); MS (ESI−): 497 ([M−H]⁻).

### Example 141

### Preparation of 5-(4-{1-Ethyl-1-[4-(3-hydroxy4,4-dimethyl-pentyl)-3-methyl-phenyl]-propyl}-2-methyl-phenoxy)4(R)-hydroxy-pentanoic acid sodium salt

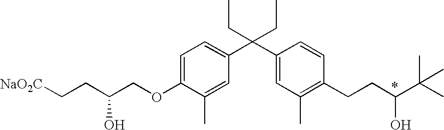

### (1) Preparation of 5(R)-(4-{1-Ethyl-1-[4-(3-hydroxy-4,4-dimethyl-pentyl)-3-methyl-phenyl]-propyl}-2-methyl-phenoxymethyl)-dihydro-furan-2-one

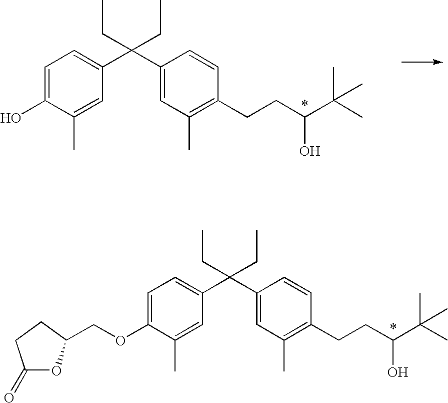

To a stirred solution of 4-{1-Ethyl-1-[4-(3-hydroxy-4,4-dimethyl-pentyl)-3-methyl-phenyl]-propyl}-2-methyl-phenol (compound prepared in Example 20) (670 mg, 1.75 mmol) in N,N-dimethylacetamide (17 ml) were added K₂CO₃ (605 mg, 4.38 mmol) and toluene-4-sulfonic acid (R)-5-oxo-tetrahydro-furan-2-ylmethyl ester (710 mg, 2.63 mmol) and the mixture was stirred at 130 degrees C. for 3 h. The reaction mixture was cooled to room temperature and poured into aqueous NH₄Cl. The products were extracted with ethyl acetate and hexane. The extracts were washed with brine, dried over MgSO₄, filtered and concentrated in vacuo. The obtained residue was chromatographed on silica gel (ethyl acetate/hexane =20/80 to 35/65) to give the title compound (330 mg, 39%).

¹H-NMR: 0.59 (t, 6H), 0.89 (s, 9H), 1.38 (d,1H), 1.44-1.55 (m, 1H), 1.74-1.84 (m, 1H), 2.03 (q, 4H), 2.15 (s, 3H), 2.25 (s, 3H), 2.27-2.62 (m, 4H), 2.72-2.91 (m, 2H), 3.21-3.27 (m, 1H), 4.044.19 (m, 2H), 4.84-4.91 (m, 1H), 6.66 (d, 1H), 6.89 -6.97 (m, 4H), 7.01 (d, 1H).

### (2) 5-(4-{1-Ethyl-1-[4-(3-hydroxy4,4-dimethyl-pentyl)-3-methyl-phenyl]-propyl}-2-methyl-phenoxy)4(R)-hydroxy-pentanoic acid sodium salt

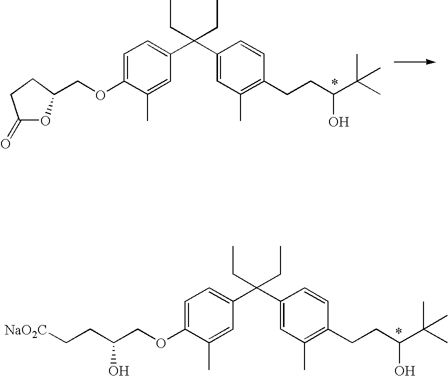

To a solution of 5-(4-{1-Ethyl-1-[4-(3-hydroxy-4,4-dimethyl-pentyl)-3-methyl-phenyl]-propyl}-2-methyl-phenoxymethyl)-dihydro-furan-2-one (compound prepared in Example 141(1)) (306 mg, 0.636 mmol) in methanol (2.5 ml) and tetrahydrofuran (2.5 ml) was added 1 N KOH solution (2.5.5 ml) and the mixture was stirred for 1 h at room temperature. The reaction mixture was concentrated in vacuo and poured into aqueous KHSO₄ and the products were extracted with AcOEt. The extracts were washed with H₂O, dried over MgSO₄, filtered and concentrated in vacuo. The obtained residue (316 mg) was dissolved with MeOH (2 ml) and 1.0M solution of NaOMe in MeOH (0.621 ml, 0.635 mmol) was added. The mixture was stirred for 2 min. and concentrated in vacuo to give the title compound (330 mg, 100%).

¹H-NMR (CD₃OD): 0.58 (t, 6H), 0.86 (s, 9H), 1.43-1.54 (m, 1H), 1.73-1.99 (m, 3H), 2.04 (q, 4H), 2.14 (s, 3H), 2.23 (s, 3H), 2.36 (dt, 2H), 2.48-2.58 (m, 1H), 2.81-2.91 (m, 1H), 3.15 (dd, 1H), 3.88-3.97 (m, 3H), 6.74 (d, 1H), 6.83-7.02 (m, 5H); MS (ESI−): 497 ([M−H]⁻).

### Example 142

### Preparation of 5-(2-Chloro4-{1-[3-chloro4-(3-hydroxy4,4-dimethyl-pentyl)-phenyl]-1-ethyl-propyl}-phenoxy)-4(R)-hydroxy-pentanoic acid sodium salt

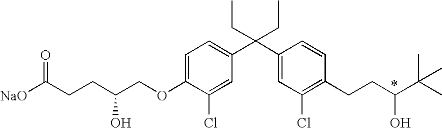

### (1) Preparation of 5(R)-(2-Chloro4-{1-[3-chloro4-(3-hydroxy4,4-dimethyl-pentyl)-phenyl]-1-ethyl-propyl}-phenoxymethyl )-dihydro-furan-2-one

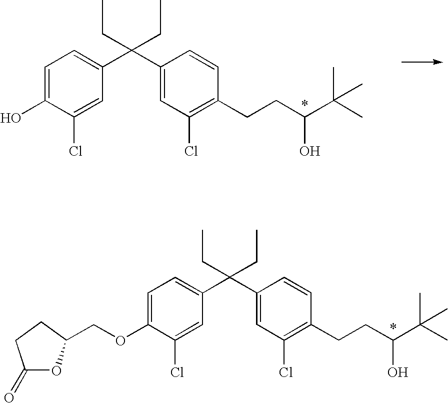

To a stirred solution of 2-Chloro-4-{1-[3-chloro-4-(3-hydroxy-4,4-dimethyl-pent-1-ynyl)-phenyl]-1-ethyl-propyl}-phenol (compound prepared in Example 16-(6)) (6 mg, 0.014 mmol) in N,N-dimethylacetamide (0.14 ml) were added K₂CO₃ (4.9 mg, 0.035 mmol) and toluene-4-sulfonic acid (R)-5-oxo-tetrahydro-furan-2-ylmethyl ester (7.6 mg, 0.028 mmol) and the mixture was stirred at 130 degrees C. for 1.5 h. The reaction mixture was cooled to room temperature and poured into aqueous NH₄Cl. The products were extracted with ethyl acetate and hexane. The extracts were washed with brine, dried over MgSO₄, filtered and concentrated in vacuo. The obtained residue was purified by preparative TLC (ethyl acetate/hexane=2/3) to give the title compound (6.0 mg, 81%).

¹H-NMR: 0.61 (t, 6H), 0.89 (s, 9H), 1.42-1.59 (m, 2H), 1.81-1.92 (m,1H), 2.02 (q, 4H), 2.35-2.75 (m, 4H), 2.90-3.02 (m, 2H), 3.21-3.31 (m,1H), 4.07-4.27 (m, 2H), 4.86-4.91 (m, 1H), 6.79 (d, 1H), 6.91 (dd, 1H), 6.98 (dd, 1H), 7.11-7.19 (m, 3H).

### (2) Preparation of 5-(2-Chloro-4-{1-[3-chloro4-(3-hydroxy-4,4-dimethyl-pentyl)-phenyl]-1-ethyl-propyl}-phenoxy)-4(R)-hydroxy-pentanoic acid sodium salt

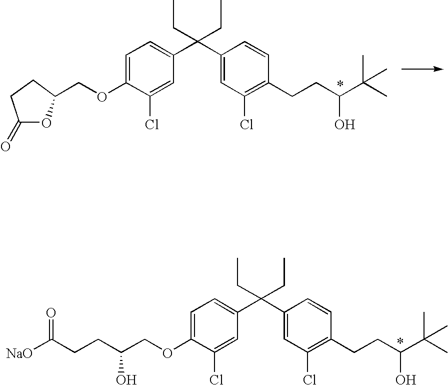

To a solution of (5R)-(2-Chloro-4-{1-[3-chloro-4-(3-hydroxy-4,4-dimethyl-pentyl)-phenyl]-1-ethyl-propyl}-phenoxymethyl)-dihydro-furan-2-one (compound prepared in Example 142-(1)) (6.0 mg, 0.012 mmol) in methanol (0.5 ml) and tetrahydrofuran (0.5 ml) was added 1 N KOH solution (0.5 ml) and the mixture was stirred for 1 h at room temperature. The reaction mixture was concentrated in vacuo and poured into aqueous KHSO₄ and the products were extracted with AcOEt. The extracts were washed with H₂O, dried over MgSO₄, filtered and concentrated in vacuo. The obtained residue (6.1 mg) was dissolved with MeOH (0.5 ml) and 1.0M solution of NaOMe in MeOH (0.0111 ml, 0.0114 mmol) was added. The mixture was stirred for 2 min. and concentrated in vacuo to give the title compound (6.2 mg, 96%).

¹H-NMR (CD₃OD): 0.62 (t, 6H), 0.86 (s, 9H), 1.28-1.98 (m, 4H), 2.06 (q, 4H), 2.37 (t, 2H), 2.64-2.74 (m, 1H), 2.92-3.01 (m, 1H), 3.12-3.15 (m, 1H), 3.97 (s, 3H), 6.96-7.20 (m, 6H); MS (ESI−): 537 ([M−H]⁻).

### Example 143

### Preparation of 5-(2-Chloro-4-{1-[3-chloro-4-(3-hydroxy4,4-dimethyl-pentyl)-phenyl]-1-ethyl-propyl}-phenoxy)-4(R)-hydroxy-pentanoic acid sodium salt

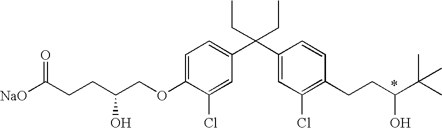

### (1) Preparation of 5(R)-(2-Chloro-4-{1-[3-chloro4-(3-hydroxy4,4-dimethyl-pentyl)-phenyl]-1-ethyl-propyl}-phenoxymethyl)-dihydro-furan-2-one

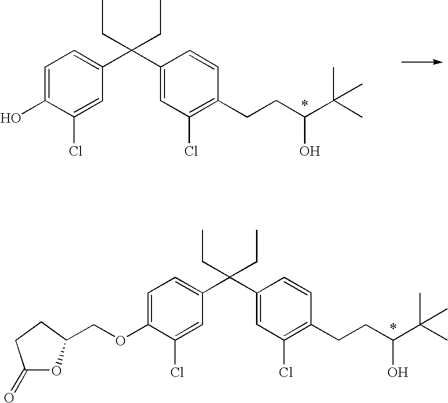

To a stirred solution of 2-Chloro-4-{1-[3-chloro4-(3-hydroxy4,4-dimethyl-pent-1-ynyl)-phenyl]-1-ethyl-propyl}-phenol (compound prepared in Example 17) (6 mg, 0.014 mmol) in N,N-dimethylacetamide (0.14 ml) were added K₂CO₃ (4.9 mg, 0.035 mmol) and toluene-4-sulfonic acid (R)-5-oxo-tetrahydro-furan-2-ylmethyl ester (7.6 mg, 0.028 mmol) and the mixture was stirred at 130 degrees C. for 1.5 h. The reaction mixture was cooled to room temperature and poured into H₂O. The products were extracted with ethyl acetate and hexane. The extracts were washed with brine, dried over MgSO₄, filtered and concentrated in vacuo. The obtained residue was purified by preparative TLC (ethyl acetate/hexane=2/3) to give the title compound (6.0 mg, 81%).

¹H-NMR: 0.61 (t, 6H), 0.89 (s, 9H), 1.42-1.59 (m, 2H), 1.82-1.91 (m, 1H), 2.02 (q, 4H), 2.35-2.76 (m, 4H), 2.90-3.02 (m, 2H), 3.21-3.30 (m, 1H), 4.07-4.27 (m, 2H), 4.86-4.91 (m, 1H), 6.80 (d, 1H), 6.91 (dd, 1H), 6.98 (dd, 1H), 7.11-7.17 (m, 3H).

### (2) 5-(2-Chloro-4-{1-[3-chloro-4-(3-hydroxy4,4-dimethyl-pentyl)-phenyl]-1-ethyl-propyl}-phenoxy)-4(R)-hydroxy-pentanoic acid sodium salt

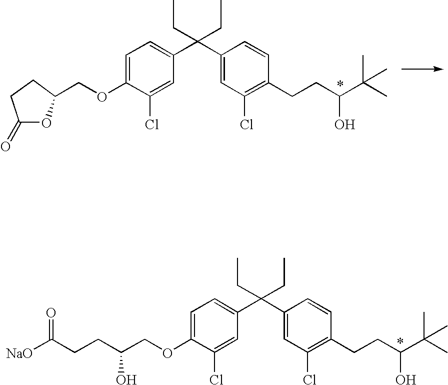

To a solution of (5R)-(2-Chloro-4-{1-[3-chloro-4-(3-hydroxy-4,4-dimethyl-pentyl)-phenyl]-1-ethyl-propyl}-phenoxymethyl)-dihydro-furan-2-one(compound prepared in Example 143-(1)) (6.0 mg, 0.012 mmol) in methanol (0.5 ml) and tetrahydrofuran (0.5 ml) was added 1 N KOH solution (0.5 ml) and the mixture was stirred for 1 h at room temperature. The reaction mixture was concentrated in vacuo and poured into aqueous KHSO₄ and the products were extracted with AcOEt. The extracts were washed with H₂O, dried over MgSO₄, filtered and concentrated in vacuo. The obtained residue (6.3 mg) was dissolved with MeOH (0.5 ml) and 1.0M solution of NaOMe in MeOH (0.0114 ml, 0.0117 mmol) was added. The mixture was stirred for 2 min. and concentrated in vacuo to give the title compound (6.6 mg, 96%).

¹H-NMR (CD₃OD): 0.62 (t, 6H), 0.86 (s, 9H), 1.28-2.01 (m,.4H), 2.06 (q, 4H), 2.37 (t, 2H), 2.63-2.74 (m, 1H), 2.92-3.01 (m, 1H), 3.13 (dd, 1H), 3.97 (s, 3H), 6.96-7.20 (m, 6H); MS (ESI−): 537 ([M−H]⁻).

### Example 144

### Preparation of (R)-5-(4-{1-Ethyl-1-[4-((E)-3-ethyl-3-hydroxy-pent-1-enyl)-3-methyl-phenyl]-propyl}-2-methyl-phenoxymethyl)-pyrrolidin-2-one

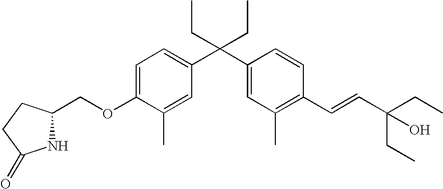

### Preparation of (R)-5-(4-{1-Ethyl-1-[4-((E)-3-ethyl-3-hydroxy-pent-1-enyl)-3-methyl-phenyl]-propyl}-2-methyl-phenoxymethyl)-pyrrolidin-2-one

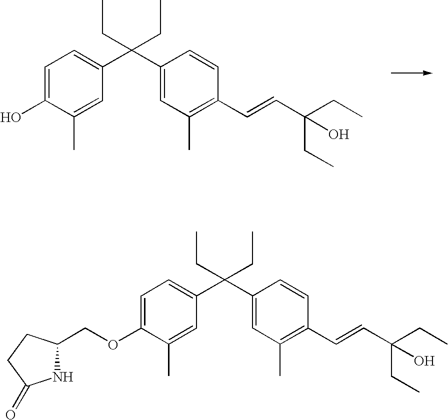

To a solution of 4-{1-Ethyl-1-[4-((E)-3-ethyl-3-hydroxy-pent-1-enyl)-3-methyl-phenyl]-propyl}-2-methyl-phenol (compound prepared in Example 1-(5)) (300 mg, 0.788 mmol) in MeCN (5 ml), toluene-4-sulfonic acid (R)-5-oxo-pyrrolidin-2-ylmethyl ester (425 mg, 1.577 mmol) and K₂CO₃ (545 mg, 3.942 mmol) were added at room temperature and the mixture was stirred at 108 degrees C. for 24 h. To the mixture, ethyl acetate was added and the mixture was washed with brine, dried over MgSO₄, filtered and concentrated in vacuo. The obtained residue was chromatographed on silica gel (n-hexane:EtOAc=100:0 to 0:100) to give the title compound (159.2 mg, 42.3%).

¹H-NM R (CDCl₃): 0.61 (t, 6H, J=7.2 Hz), 0.92 (t, 6H, J=7.5 Hz), 1.64-1.60 (q, 4H, J=7.5 Hz), 1.86-2.00 (m, 1H), 2.05 (q,-4H, J=7.2 Hz), 2.15 (s, 3H), 2.31 (s, 3H), 4.07-4.13 (m, 3H), 3.31 (dd, 2H, J=7.5, 9.3 Hz), 3.97 (dd, 1H, J=3.8, 9.3 Hz), 4.07-4.13 (m, 1H), 6.01 (d, 1H, J=15.9 Hz), 6.64 (d, 1H, J=8.4 Hz), 6.74 (d, 1H, J=15.9 Hz), 6.90-6.96 (m, 4H), 7.29 (d,1H, J=8.7 Hz); MS (ESI+): 478 ([M+H]⁺).

### Example 145

### Preparation of (S)-5-(4-{1-Ethyl-1-[4-((E)-3-ethyl-3-hydroxy-pent-1-enyl)-3-methyl-phenyl]-propyl}-2-methyl-phenoxymethyl )-pyrrolidin-2-one

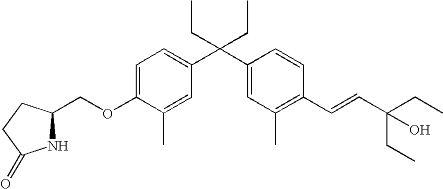

### Preparation of (S)-5-(4-{1-Ethyl-1-[4-((E)-3-ethyl-3-hydroxy-pent-1-enyl)-3-methyl-phenyl]-propyl}-2-methyl-phenoxymethyl)-pyrrolidin-2-one

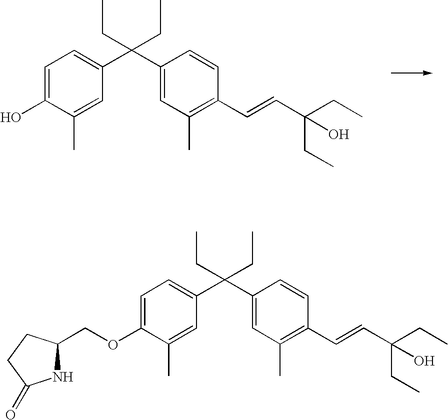

To a solution of 4-{1-ethyl-1-[4-((E)-3-ethyl-3-hydroxy-pent-1-enyl)-3-methyl-phenyl]-propyl}-2-methyl-phenol (compound prepared in Example 1-(5)) (300 mg, 0.788 mmol) in MeCN (5 ml), toluene-4-sulfonic acid (S)-5-oxo-pyrrolidin-2-ylmethyl ester (425 mg, 1.577 mmol) and K₂CO₃ (545 mg, 3.942 mmol) were added at room temperature and the mixture was stirred at 108 degrees C. for 24 h. To the mixture, ethyl acetate was added and the mixture was washed with brine, dried over magnesium sulfate, concentrated in vacuo. The obtained residue was chromatographed on silica gel (n-hexane:EtOAc=100:0 to 0:100) to give the title compound (152.6 mg, 40.5%).

¹H-NMR (chloroform-d): 0.61 (t, 6H, J=7.2 Hz), 0.92 (t, 6H, J=7.5 Hz), 1.64 (q, 4H, J=7.5 Hz), 1.67 (s, 1H), 1.88-1.99 (m, 1H), 2.05 (q, 4H, J=7.2 Hz), 2.15 (s, 3H), 2.31 (s, 3H), 2.27-2.52 (m, 2H), 3.81 (dd,1H, J=7.5, 9.3 Hz), 3.97 (dd,1H, J=3.8, 9.3 Hz), 4.06-4.13 (m, 1H), 5.99 (brs, 1H), 6.01 (d, 1H, J=1 5.9 Hz), 6.64 (d, 1H, J=8.1 Hz), 6.74 (d,1H), 6.89-6.98 (m, 4H), 7.29 (d,1H); MS (ESI+): 478 ([M+H]⁺).

### Example 146

### Preparation of 3-(4-{1-Ethyl-1-[4-((E)-3-ethyl-3-hydroxy-pent-1-enyl)-3-methyl-phenyl]-propyl}-2-methyl-phenoxymethyl)-benzoic acid

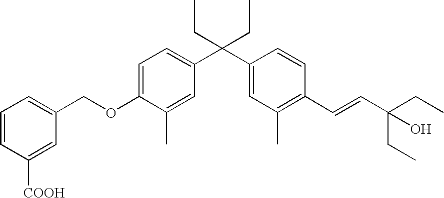

### Preparation of 3-(4-{1-Ethyl-1-[4-((E)-3-ethyl-3-hydroxy-pent-1-enyl)-3-methyl-phenyl]-propyl}-2-methyl-phenoxymethyl)-benzoic acid

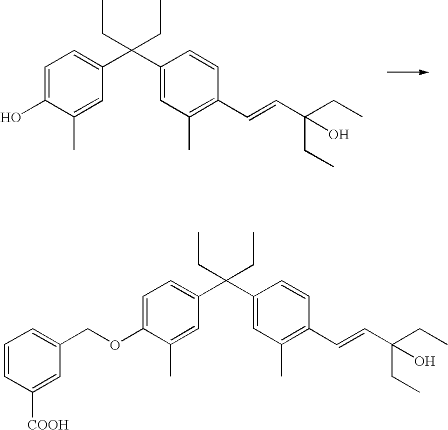

To a solution of compound prepared in Example 1-(5) (4-{1-Ethyl-1-[4-(3-ethyl-3-hydroxy-(1E)-pent-1-enyl)-3-methyl-phenyl]-propyl}-2-methyl-phenol ) (20 mg, 0.0526 mmol) in DMF (0.4 ml), K₂CO₃ (21.8 mg, 0.158 mmol ) and 3-bromomethyl-benzoic acid methyl ester (14.5 mg, 0.0632 mmol) were added at room temperature and the mixture was stirred at 120 degrees C. for 16 h. To the mixture, ethyl acetate was added and the mixture was washed with H₂O and brine, dried over MgSO₄, concentrated in vacuo. The obtained residue was dissolved in MeOH (0.5 ml), and 1 N NaOH (0.2 ml) was added at room temperature and the mixture was stirred at 45 degrees C. for 11 h. The mixture was evaporated in vacuo and the obtained residue was chromatographed on silica gel (n-hexane:EtOAc=100:0 to 0:100) and re-purified by preparative TLC (CHCl₃:MeOH=8:3 saturated with H₂O) to give the title compound (4.7 mg, 17%).

¹H-NMR: 0.62 (t, 6H),;0.92 (t, 6H), 1.62 (q, 4H), 2.03 (q, 4H), 2.20 (s, 3H), 2.34 (s, 3H), 5.06 (s, 2H), 6.01 (d, 1H), 6.68 (d, 1H), 6.78 (s, 1H), 7.50 (t, 1H), 7.68 (d, 1H), 8.02 (d, 1H), 8.12 (s, 1H); MS (ESI+): 532 ([M+NH₄]⁺).

### Example 147

### Preparation of 4-(4-{I-Ethyl-1-[4-((E)-3-ethyl-3-hydroxy-pent-1-enyl)-3-methyl-phenyl]-propyl}-2-methyl-phenoxymethyl)-benzoic acid

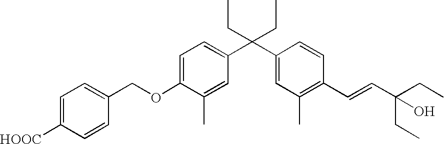

### (1) Preparation of 4-(4-{1-Ethyl-I -[4-((E)-3-ethyl-3-hydroxy-pent-1-enyl)-3-methyl-phenyl]-propyl}-2-methyl-phenoxymethyl)-benzoic acid methyl ester

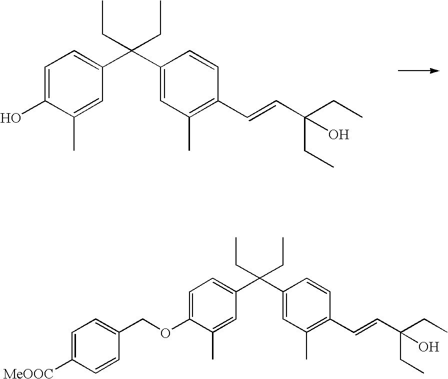

To a solution of compound prepared in Example 1-(5) (20 mg, 0.0526 mmol) (4-{1-Ethyl-1-[4-(3-ethyl-3-hydroxy-(1 E)-pent-1-enyl)-3-methyl-phenyl]-propyl}-2-methyl-phenol) in DMF (0.5 ml), K₂CO₃ (29 mg, 0.21 mmol ) and 4-bromomethyl-benzoic acid methyl ester (14.4 mg, 0.0631 mmol) were added at room temperature and the mixture was stirred for 16 h. To the mixture, ethyl acetate was added and the mixture was washed with H₂O, dried over MgSO₄, filtered and concentrated in vacuo. The obtained residue was purified by prerparative TLC (ethyl acetate/hexane=1/1) to give the title compound (25.3 mg, 91%).

¹H-NMR: 0.61 (t, 6H), 0.92 (t, 6H), 1.64 (q, 4H), 2.03 (q, 4H), 2.24 (s, 3H), 2.31 (s, 3H), 3.93 (s, 3H), 5.09 (s, 2H), 6.00 (d,1H), 6.71-6.77 (m, 2H), 6.92-6.965 (m, 4H), 7.50 (d, 2H), 8.04 (d, 2H); MS (ESI+): 511 ([M−OH]⁺).

### (2) Preparation of 3-(4-{1-Ethyl-1-[4-((E)-3-ethyl-3-hydroxy-pent-1-enyl)-3-methyl-phenyl]-propyl}-2-methyl-phenoxymethyl)-benzoic acid

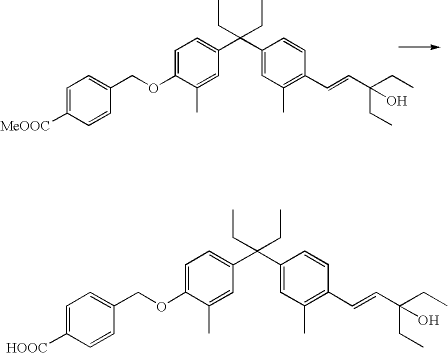

To a solution of 4-(4-{1-Ethyl-1-[4-((E)-3-ethyl-3-hydroxy-pent-1-enyl)-3-methyl-phenyl]-propyl}-2-methyl-phenoxymethyl)-benzoic acid methyl ester (compound prepared in Example 147-(1)) (25.3 mg, 0.0479 mmol) in EtOH (2 ml), 1 NaOH (0.1 ml) was added at room temperature, and the-mixture was stirred at 40 degrees C. for 16 h and concentrated in vacuo. The obtained residue was purified by preparative TLC (CHCl3:MeOH=8:3 saturated with H₂O) to give the title compound (19.0 mg, 77%).

¹H-NMR: 0.61 (t, 6H), 0.92 (t, 6H), 1.66 (q, 4H), 2.05 (q, 4H), 2.05 (s, 3H), 2.31 (s, 3H), 5.12 (s, 2H), 6.00 (d, 1H), 6.70-6.80 (m, 2H), 7.54 (d, 2H), 8.11 (d, 2H); MS (ESI+): 497([M−OH]⁺).

### Example 148

### Preparation of (E)-3-Ethyl-1-(4-{1-ethyl-1-[3-methyl-4-(pyridin-3-ylmethoxy)-phenyl]-propyl}-2-methyl-phenyl)-pent-1-en-3-ol

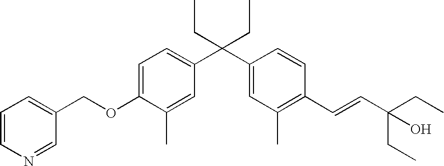

### Preparation of (E)-3-Ethyl-1-(4-{1-ethyl-1-[3-methyl-4-(pyridin-3-ylmethoxy)-phenyl]-propyl}-2-methyl-phenyl)-pent-1-en-3-ol

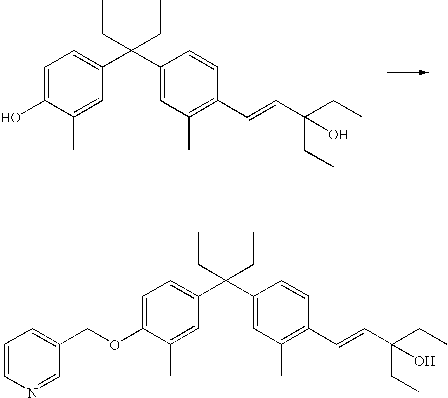

To a solution of compound prepared in Example 1-(5) (4-{1-Ethyl-1-[4-(3-ethyl-3-hydroxy-(1 E)-pent-1-enyl)-3-methyl-phenyl]-propyl}-2-methyl-phenol) (20 mg, 0.0526 mmol) in DMF (0.5 ml), K₂CO₃ (60 mg, 0.435 mmol) and 3-bromomethyl-pyridine hydrochloride (25.0 mg, 0.099 mmol) were added and the mixture was stirred at 110 degrees C. for 80 h. To the mixture, ethyl acetate was added and the mixture was washed with H₂O and brine, dried over MgSO₄, concentrated in vacuo. The obtained residue was purified by prerparative TLC (ethyl acetate:hexane=1:3) to give the title compound (11.7 mg, 47%).

¹H-NMR: 0.61 (t, 6H), 0.92 (t, 6H), 1.63 (q, 4H), 2.04 (q, 4H), 2.22 (s, 3H), 2.32 (s, 3H), 5.06 (s, 2H), 6.01 (d,1H), 6.72-6.88 (m, 2H), 7.24-7.37 (m, 3H), 7.80 (d, 1H), 8.57 (d,1H), 8.60 (s,1H); MS (ESI+): 454 ([M−OH]⁺).

### Example 149

### Preparation of (E)-3-Ethyl-1-(4-{1-ethyl-1-[3-methyl-4-(pyridin4-ylmethoxy)-phenyl]-propyl}-2-methyl-phenyl)-pent-1-en-3-ol

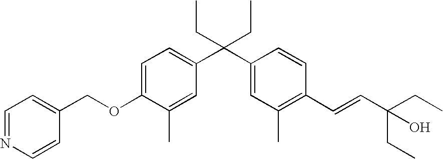

### Preparation of (E)-3-Ethyl-1-(4-{1-ethyl-1-[3-methyl4-(pyridin4-ylmethoxy)-phenyl]-propyl}-2-methyl-phenyl)-pent-1-en-3-ol

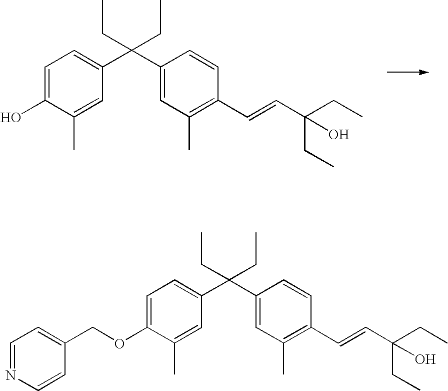

To a solution of compound prepared in Example 1-(5) (4-{1-Ethyl-1-[4-(3-ethyl-3-hydroxy-(1 E)-pent-1-enyl)-3-methyl-phenyl]-propyl}-2-methyl-phenol ) (20 mg, 0.0526 mmol) in DMF (0.5 ml), K₂CO₃ (29 mg, 0.210 mmol) and 4-chloromethyl-pyridine hydrochloride (10.3 mg, 0.063 mmol) were added and the mixture was stirred at room temperature for 16 h. To the mixture, ethyl acetate was added and the mixture was washed with H₂O and brine, dried over MgSO₄, filtered and concentrated in vacuo. The obtained residue was purified by prerparative TLC (ethyl acetate:hexane=1:3) to give the title compound (17.8 mg, 72%).

¹H-NMR: 0.61 (t, 6H), 0.92 (t, 6H), 1.64 (q, 4H), 2.05 (q, 4H), 2.26 (s, 3H), 2.31 (s, 3H), 5.06 (s, 2H), 6.01 (d, 1H), 6.70-6.80 (m, 2H), 6.92-6.97 (m, 4H), 7.25 (d, 1H), 7.38 (d, 2H), 8.62 (d, 2H); MS (ESI+): 454 ([M−H]⁺).

### Example 150

### Preparation of 4-(4-{1-Ethyl-1-[4-((E)-3-ethyl-3-hydroxy-pent-1-enyl)-3-methyl-phenyl]-propyl}-2-methyl-phenoxy)-benzoic acid methyl ester

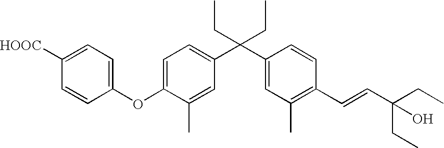

### (1) Preparation of 4-(4-{1-Ethyl-1-[4-((E)-3-ethyl-3-hydroxy-pent-1-enyl)-3-methyl-phenyl]-propyl)}-2-methyl-phenoxy)-benzoic acid methyl ester

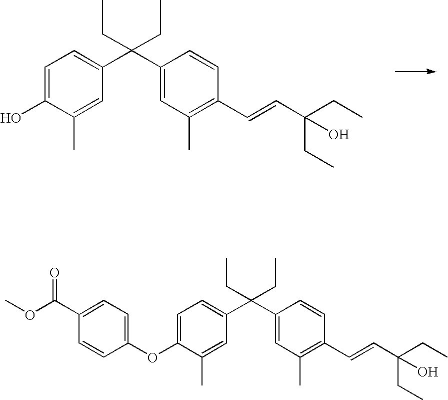

To a solution of compound prepared in Example 1-(5) (4-{1-Ethyl-1-[4-(3-ethyl-3-hydroxy-(1 E)-pent-1-enyl)-3-methyl-phenyl]-propyl}-2-methyl-phenol) (20 mg, 0.0526 mmol) in DMF (0.5 ml), K₂CO₃ (21.8 mg, 0.158 mmol ) and 4-fluoro-benzoic acid methyl ester (13.3 mg, 0.0789 mmol) were added and the mixture was stirred at 110 degrees C. for 16 h. To the mixture, ethyl acetate was added and the mixture was washed with H₂O and brine, dried over MgSO₄, filtered and concentrated in vacuo. The obtained residue was purified by prerparative TLC (ethyl acetate:hexane=1:3) to give the title compound (19.0 mg, 70%).

¹H-NMR: 0.64 (t, 6H), 0.92 (t, 6H), 1.62 (q, 4H), 2.05-2.11 (m, 7H), 2.33 (s, 3H), 3.88 (s, 3H), 6.02 (d, 1H), 6.73-7.34 (m, 9H), 7.96 (d, 2H); MS (ESI+): 515 ([M+H]⁺).

### (2) Preparation of 4-(4-{1-Ethyl-1-[4-((E)-3-ethyl-3-hydroxy-pent-1-enyl)-3-methyl-phenyl]-propyl}-2-methyl-phenoxy)-benzoic acid

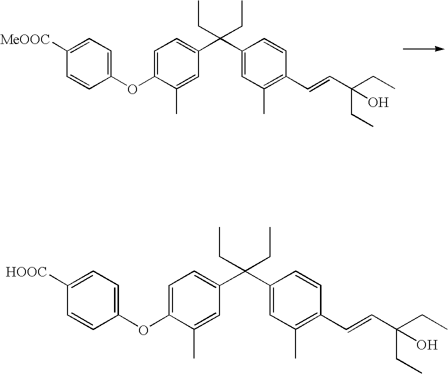

To a solution of 4-(4-{1-Ethyl-1-[4-((E)-3-ethyl-3-hydroxy-pent-1-enyl)-3-methyl-phenyl]-propyl}-2-methyl-phenoxy)-benzoic acid methyl ester (compound prepared in Example 150-(1)) (17 mg, 0.0331 mmol) in EtOH (2 ml), 1N NaOH (0.1 ml) was added, and the mixture was stirred at 50 degrees C. for 16 h and concentrated in vacuo. The obtained residue was purified by preparative TLC (CHCl₃:MeOH=8:3 saturated with H₂O and ethyl acetate:hexane=1:1 saturated with H₂O) to give the title compound (3.2 mg, 19%).

¹H-NMR: 0.65 (t, 6H), 0.93 (t, 6H), 1.66 (q, 4H), 2.05-2.34 (m, 7H), 2.34 (s, 3H), 6.03 (d,1H), 6.73-7.35 (m, 9H), 8.02 (d, 2H); MS (ESI+): 518 ([M+NH₄]⁺).

### Example 151

### Preparation of (E)-(4-{1-Ethyl-1-[4-(3-ethyl-3-hydroxy-pent-1-enyl)-3-methyl-phenyl]-propyl}-2-methyl-phenoxy)-acetic acid

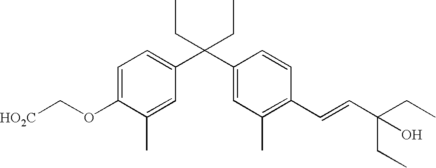

### (1) Preparation of (E)-(4-{1-Ethyl-1-[4-(3-ethyl-3-hydroxy-pent-1-enyl)-3-methyl-phenyl]-propyl}-2-methyl-phenoxy)-acetic acid methyl ester

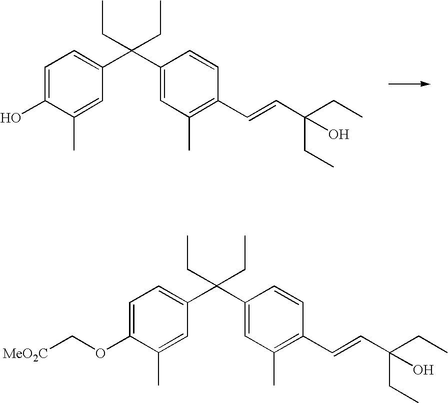

To a stirred solution of 4-{1-Ethyl-1-[4-(3-ethyl-3-hydroxy-pent-1-enyl)-3-methyl-phenyl]-propyl}-2-methyl-phenol (compound prepared in Example 1-(5)) (700 mg, 1.84 mmol) in acetone (18 ml) were added K₂CO₃ (763 mg, 5.52 mmol) and bromoaceticacid methyl ester (563 mg, 3.68 mmol) at room temperature and the mixture was refluxed for 4. h. The reaction mixture was concentrated and poured into H₂O and the products were extracted with ethyl acetate. The extracts were washed with brine, dried over MgSO₄, filtered and concentrated in vacuo. The obtained residue was chromatographed on silica gel (ethyl acetate/hexane=0/100 to 5/95) to give the title compound (892 mg, quant.).

¹H-NMR: 0.61 (t, 6H), 0.92 (t, 6H), 1.64 (q, 4H), 2.05 (q, 4H), 2.23 (s, 3H), 2.31 (s, 3H), 3.80 (s, 3H), 4.62 (s, 2H), 6.02 (d,1H), 6.57 (d,1H), 6.75 (d, 1H), 6.92-6.94 (m, 4H) 7.30 (d, 1H).

### (2) Preparation of (E)-(4-{l-Ethyl-1-[4-(3-ethyl-3-hydroxy-pent-1-enyl)-3-methyl-phenyl]-propyl}-2-methyl-phenoxy)-acetic acid

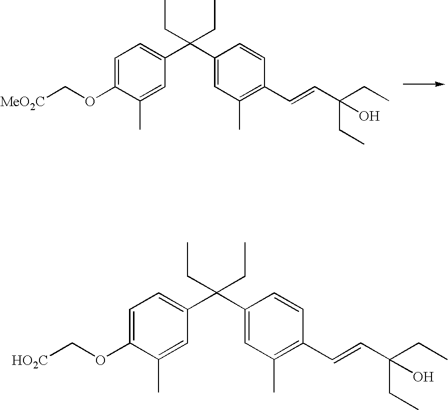

To a solution of (4-{1-Ethyl-1-[4-(3-ethyl-3-hydroxy-pent-1-enyl)-3-methyl-phenyl]-propyl}-2-methyl-phenoxy)-acetic acid methyl ester (compound prepared in Example 151-(1)) (892 mg, 1.97 mmol) in methanol (3 ml) and tetrahydrofuran (2 ml) was added 1 N KOH solution (3 ml) and the mixture was stirred for 2 h at room temperature. The reaction mixture was poured into aqueous KHSO₄, and the products were extracted with AcOEt. The extracts were washed with brine, dried over MgSO₄, filtered and concentrated in vacuo to give the title compound (807 mg, 100.%).

¹H-NMR: 0.60 (t, 6H), 0.92 (t, 6H), 1.64 (q, 4H), 2.05 (q, 4H), 2.23 (s, 3H), 2.31 (s, 3H), 4.65 (s, 2H), 6.01 (d, 1H), 6.62 (d, 1H), 6.75 (d,1H), 6.92-6.97 (m, 4H) 7.29 (d,1H).

### Example 152

### Preparation of (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol

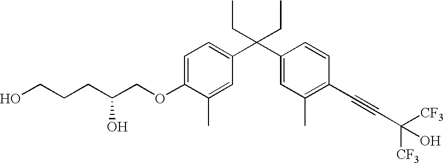

### Preparation of (R)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-pentane-1,4-diol

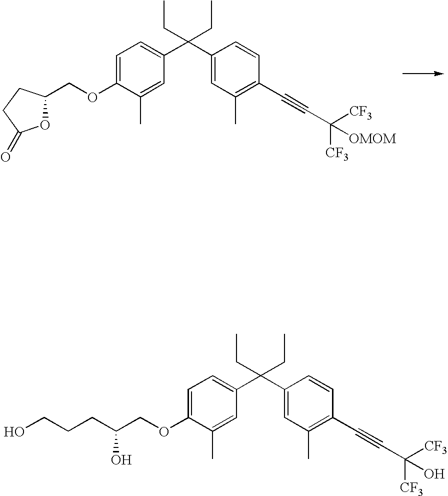

To a solution of (R)-5-(4-{1-ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one (compound prepared in Example 46-(1)) (39 mg, 0.065 mmol) in Et₂O (1 ml), LAH (7 mg, 0.195 mmol) was added and the mixture was stirred at room temperature for 18 h. To the mixture, ethyl acetate and brine were added and the organic layer was filtered through celite, and the filtrate was concentrated in vacuo. To the solution of the residue (7.3 mg) in i-PrOH (0.1 ml), CBr₄ (0.33 mg 0.001 mmol) was added and the mixture was stirred at 85 degrees C. for 3.5 h. The mixture was purified by silica gel chromatography (n-hexane/ethyl acetate=1/1) to give the title compound (7.2 mg, 91.7%).

¹H-NMR (chloroform-d): 0.59 (t, 6H, J=7.2 Hz), 1.67-1.83 (m, 4H), 2.04 (q, 4H, J=7.2 Hz), 2.14 (s, 3H), 2.37 (s, 3H), 2.83 (brs, 1H), 3.66-3.77 (m, 2H), 3.81 (dd, 1H, J=7.4, 9.2 Hz), 3.92 (dd, 1H, J=3.5, 9.2 Hz), 3.86-4.09 (m, 1H), 4.72 (brs, 1H), 6.65 (d,1H, J=8.6 Hz), 6.83-7.04 (m, 5H), 7.33 (d,1H, J=8.0 Hz); MS(ESI+): 583([M+Na]⁺).

### Example 153

### Preparation of (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol

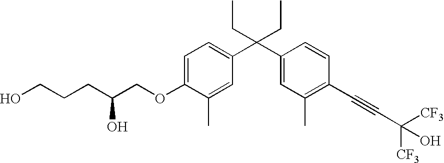

### Preparation of (S)-5-(4-{1-Ethyl-1-[3-methyl4-(4,4,4-trifluoro-3-hydroxy-3-trifluoromethyl-but-1-ynyl)-phenyl]-propyl}-2-methyl-phenoxy)-pentane-1,4-diol

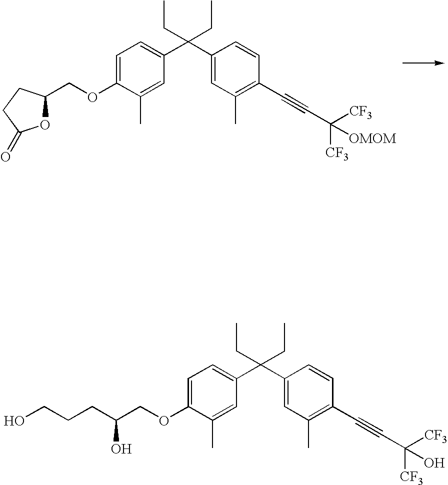

To a solution of (S)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one (compound prepared in Example 2-(3)) (38 mg, 0.063 mmol) in Et₂O (1 ml), LAH (7.2 mg 0.19 mmol) was added and the mixture was stirred at room temperature for 18 h. To the mixture, ethyl acetate and brine were added and the organic layer was filtered through celite, and the filtrate was concentrated in vacuo. To the solution of the residue (7.3 mg) in i-PrOH (0.1 ml), CBr₄ (0.33 mg 0.001 mmol) was added and the mixture was stirred at 85 degrees C. for 3.5 h. The mixture was purified by silica gel chromatography (n-hexane/ethyl acetate=1/1) to give the title compound (5 mg, 74.4%).

¹H-NMR (chloroform-d): 0.59 (t, 6H, J=7.3 Hz), 1.53-1.80 (m, 4H), 2.05 (q, 4H, J=7.3 Hz), 2.15 (s, 3H), 2.37 (s, 3H), 2.80 (brs, 1H), 3.73 (dd, 2H, J=5.0, 10.7 Hz), 3.82 (dd, 1H, J=7.4, 9.3 Hz), 3.93 (dd, 1H, J=3.6, 9.3 Hz), 4.014.10 (m, 1H), 4.31 (brs,1H), 6.66 (d,1H, J=8.5 Hz), 6.83-7.04 (m, 4H), 7.34 (d,1H, J=8.1 Hz); MS (ESI+): 583 ([M+Na]⁺).

### Example 154

### Preparation of (R)-5-(4-{1-Ethyl-1-[3-methyl4-((E)-4,4,4-trifluoro-3-hydroxy-3-trifluoromethyl-but-1-enyl)-phenyl]-propyl}-2-methyl-phenoxy)-pentane-1,4-diol

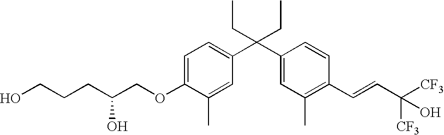

### Example 155

### Preparation of (E)4-[4-(1-Ethyl-1-{3-methyl4-[(R)-1-(tetrahydro-furan-2-yl)methoxy]-phenyl}-propyl)-2-methyl-phenyl]-11,11-trifluoro-2-trifluoromethyl-but-3-en-2-ol

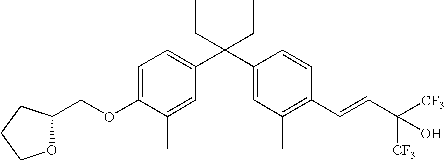

### Preparation of (R)-5-(4-{1-Ethyl-1-[3-methyl4-((E)4,4,4-trifluoro-3-hydroxy-3-trifluoromethyl-but-1-enyl)-phenyl]-propyl}-2-methyl-phenoxy)-pentane-1,4-diol and (E)-4-[4-(1-Ethyl-1-{3-methyl4-[(R)-1-(tetrahydro-furan-2-yl)methoxy]-phenyl}-propyl)-2-methyl-phenyl]-1,1,1-trifluoro-2-trifluoromethyl-but-3-en-2-ol

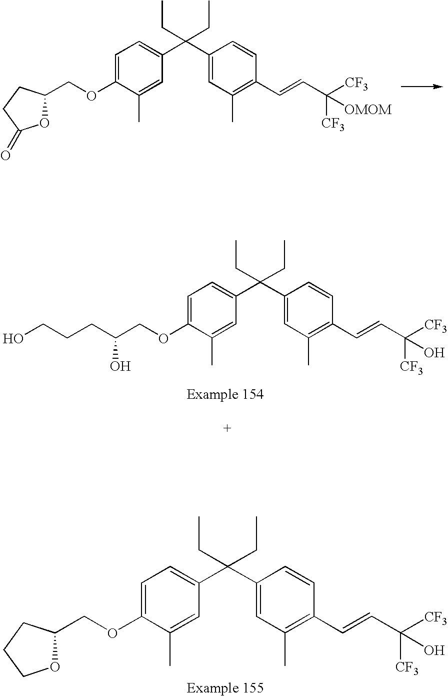

To a solution of (R)-5-(4-{1-Ethyl-1-[3-methyl4-((E)-4,4,4-trifluoro-3-methoxymethoxy-3-trifluoromethyl-but-1-enyl)-phenyl]-propyl}-2-methyl-phenoxymethyl)-dihydro-furan-2-one (compound prepared in Example 48-(1)) (39.9 mg, 0.066 mmol) in Et₂O (1 ml), LAH (7.5 mg, 0.199 mmol) was added and the mixture was stirred at room temperature for 18 h. To the mixture, ethyl acetate and brine were added and the organic layer was filtered through celite, and the filtrate was concentrated in vacuo. To the residue (25.6 mg), conc.HCl in MeOH (1 ml) was added and the mixture was stirred at 85 degrees C. for 3.5 h. The mixture was chromatographed on silica gel (n-hexane/ethyl acetate=1/1) to give the title compounds (example 154 (15 mg, 63.2%) and example 155 (3.5 mg, 15.2%)).

### Example 154

¹H-NMR (chloroform-d): 0.61 (t, 6H, J=7.2 Hz), 1.58-1.83 (m, 4H), 2.06 (q, 4H, J=7.2 Hz), 2.17 (s, 3H), 2.33 (s, 3H), 2.61 (brs, 1H), 3.72 (dd, 2H, J=5.1, 10.5 Hz), 3.84 (dd, 1H, J=7.2, 9.3 Hz), 3.95 (dd,1H, J=3.5, 9.3 Hz), 3.93 (brs,1H), 4.01-4.11 (m, 1H), 6.08 (d, 1H, J=15.9 Hz), 6.68 (d, 1H, J=8.4 Hz), 6.87-7.06 (m, 4H), 7.33 (d, 1H, J=5.7 Hz), 7.37 (d, 1H, J=1 3.2 Hz); MS (ESI+): 585 ([M+Na]⁺).

### Example 155

¹H-NMR (chloroform-d): 0.60 (t, 6H, J=7.2 Hz), 1.80-2.12 (m, 4H), 2.05 (q, 4H, J=7.2 Hz), 2.17 (s, 3H), 2.33 (s, 3H), 3.19 (brs, 1H), 3.79-4.00 (m, 4H), 4.24-4.33 (m,1H), 6.08 (d,1H, J=16.2 Hz), 6.68 (d,1H, J=8.7 Hz), 6.87-7.02 (m, 4H), 7.33 (d, 1H, J=5.1 Hz), 7.37 (d, 1H, J=12.9 Hz); MS (ESI+): 562 ([M+NH₄]⁺).

### Example 156

### Preparation of (S)-5-(4-{1-Ethyl-1-[3-methyl4-((E)4,4,4-trifluoro-3-hydroxy-3-trifluoromethyl-but-1-enyl)-phenyl]-propyl}-2-methyl-phenoxy)-pentane-1,4-diol

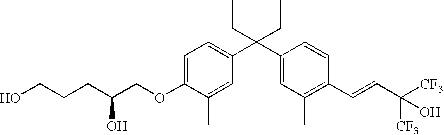

### Example 157

### Preparation of (E)4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol

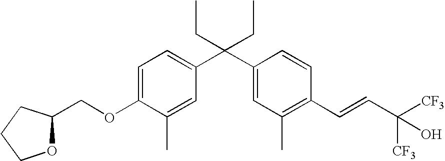

### Preparation of (S)-5-(4-{1-Ethyl-1-[3-methyl4-((E)4,4,4-trifluoro-3-hydroxy-3 -trifluoromethyl-but-1-enyl)-phenyl]-propyl}-2-methyl-phenoxy)-pentane-1,4-diol and (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol

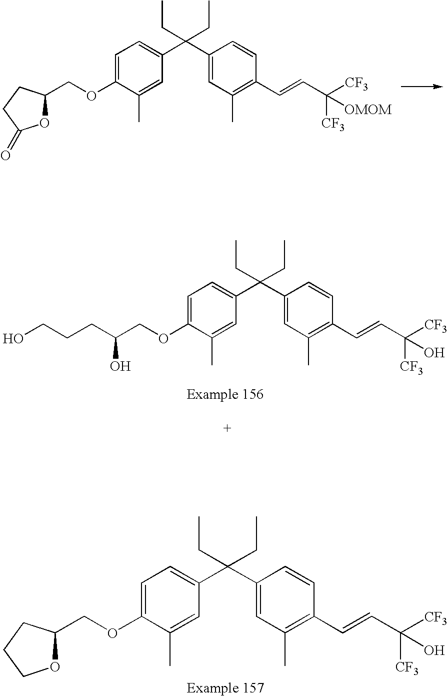

To a solution of (S)-5-(4-{1-Ethyl-1-[3-methyl4-((E)4,4,4-trifluoro-3-methoxymethoxy-3-trifluoromethyl-but-1-enyl)-phenyl]-propyl}-2-methyl-phenoxymethyl)-dihydro-furan-2-one (compound prepared in Example 30-(2)) (40.1 mg, 0.067 mmol) in Et₂O (1 ml), LAH (7.6 mg, 0.200 mmol)-was added and stirred at room temperature for 18 h. To the mixture, ethyl acetate and brine were added and the organic layer was filtered through celite, and the filtrate was concentrated in vacuo. To the residue (20.7 mg), conc.HCl in MeOH (1 ml) was added and the mixture was stirred at 85 degrees C. for 3.5 h. To the same residue (13.4 mg) in iPrOH (0.2 ml), CBr₄ (0.7 mg, 0.002 mmol) was added and the mixture was stirred at 85 degrees C. for 3.5 h. The mixture was chromatographed on silica gel (n-hexane/ethyl acetate=1/1) to give the title compounds (example 156 (21.8 mg, 68.9%) and example 157. (1 mg, 5.4%)).

### Example 156

¹H-NMR (chloroform-d): 0.61 (t, 6H, J=7.3 Hz), 1.61-1.83 (m, 4H), 2.06 (q, 4H, J=7;3 Hz), 2.17 (s, 3H), 2.33 (s, 3H), 2.54 (brs, 1H), 2.89 (brs, 1H), 3.77-3.67 (m, 2H), 3.84 (dd,1H, J=7.4, 9.3 Hz), 0.00 (dd,1H, J=3.6, 9.3 Hz), 4.01-4.09 (m,1H), 4.25 (brs, 1H), 6.08 (d, 1H, J=15.8 Hz), 6.68 (d,1H, J=8.5 Hz), 6.89-7.01 (m, 4H), 7.37 (d, 1H, J=15.8 Hz), 7.34 (d, 1H, J=7.9 Hz); MS (ESI+): 585([M+Na]⁺).

### Example 157

¹H-NMR (chloroform-d): 0.61 (t, 6H), 1.83-2.16 (m, 4H), 2.05 (q, 4H, J=7.3 Hz), 2.17 (s, 3H), 2.33 (s, 3H), 3.20 (brs, 1H), 3.79-4.01 (m, 4H), 4.25-4.33 (m, 1H), 6.08 (d, 1H, J=15.3 Hz), 6.68 (d, 1H, J=8.7 Hz), 6.87-6.97 (m, 2H), 6.99-7.03 (m, 2H), 7.33 (d, 1H, J=7.8 Hz), 7.36 (d, 1H, J=15.3 Hz); MS (ESI+): 567([M+Na]⁺).

### Example 158

### Preparation of (R)-5-(4-{1-Ethyl-1-[4-((E)-3-ethyl-3-hydroxy-pent-1-enyl)-3-methyl-phenyl]-propyl}-2-methyl-phenoxy)-pentane-1,4-diol

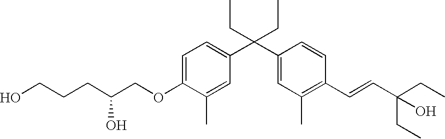

### Preparation of (R)-5-(4-{1-Ethyl-1-[4-((E)-3-ethyl-3-hydroxy-pent-1-enyl)-3-methyl-phenyl]-propyl}-2-methyl-phenoxy)-pentane-1,4-diol

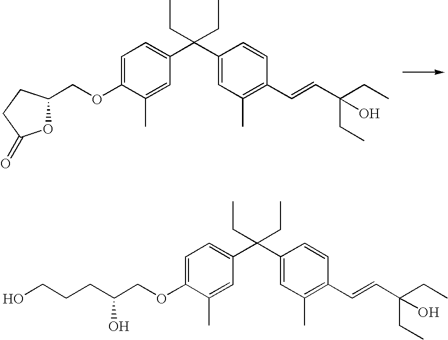

To a solution of (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one (compound prepared in Example 44) (55.7 mg, 0.116 mmol) in Et₂O (3 ml), LAH (13 mg, 0.349 mmol) was added and the mixture was stirred at room temperature for 18 h. To the mixture, ethyl acetate and brine were added and the organic layer was filtered through celite, and the filtrate was concentrated in vacuo. The obtained residue was chromatographed on silica gel (n-hexane/ethyl acetate=1/3 to ethyl acetate) to give the title compound (18.7 mg, 33.3%).

¹H-NMR (chloroform-d): 0.61 (t, 6H, J=7.4 Hz), 0.92 (t, 6H, J=7.8 Hz), 0.00 (q, 4H, J=7.8 Hz), 1.70-1.81 (m, 4H), 2.05 (q, 4H, J=7.4 Hz), 2.18 (s, 3H), 2.31(s, 3H), 2.81 (brs, 1H), 3.65 (brs,1H), 3.68-3.78 (m, 2H), 3.83 (dd, ₁H, J=7.4, 9.3 Hz), 3.95 (dd,1H, J=3.6, 9.3 Hz), 4.02-4.11 (m,1H), 6.01 (d,1H, J=15.9 Hz), 6.68 (d,1H, J=8.4 Hz), 6.74 (d,1H, J=15.9 Hz), 6.91-6.97 (m, 4H), 7.30 (d,1H, J=8.7 Hz); MS (ESI+): 500 ([M+NH₄]⁺).

### Example 159

### Preparation of (E)-3-Ethyl-1-[4-(1-ethyl-1-{3-methyl-4-[(R)-1-(tetrahydro-furan-2-yl)methoxy]-phenyl}-propyl)-2-methyl-phenyl]-pent-1-en-3-ol

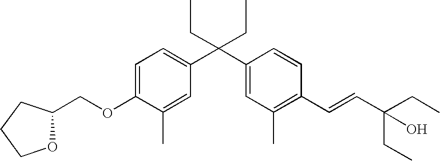

### Preparation of (E)-3-Ethyl-1-[4-(1-ethyl-1-{3-methyl-4-[(R)-1-(tetrahydro-furan-2-yl)methoxy]-phenyl}-propyl)-2-methyl-phenyl]-pent-1-en-3-ol

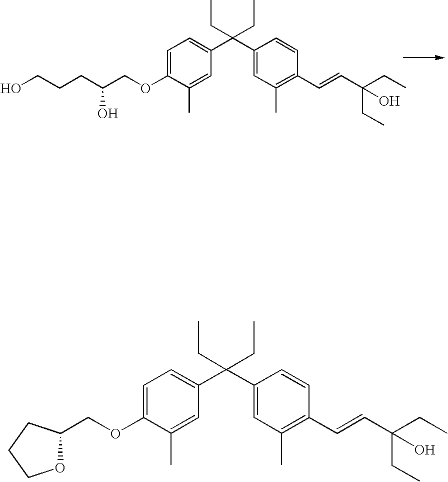

To a solution of (R)-5-(4-{1-Ethyl-1-[4-((E)-3-ethyl-3-hydroxy-pent-1-enyl)-3-methyl-phenyl]-propyl}-2-methyl-phenoxy)-pentane-1,4-diol (compound prepared in Example 158) (30 mg, 0.062 mmol) in THF (0.5 ml), PPh₃ (48.91 mg, 0.186 mmol), phthalimide (27.43 mg, 0.186 mmol), DEAD (32.47 mg, 0.186 mmol) were added and the mixture was stirred at room temperature for 4 h. The mixture was concentrated in vacuo, and the obtained residue was purified by preparative TLC (n-hexane/ethyl acetate=3/1 and 1/1) to give the title compound (22.1 mg, 76.5%).

¹H-NMR (chloroform-d): 0.58 (t, 6H, J=7.5 Hz), 0.92 (t,-6H, J=7.2 Hz), 1.64 (q, 4H, J=7.2 Hz), 1.80-2.15 (m, 4H), 2.05 (q, 4H, J=7.5 Hz), 2.18 (s, 3H), 2.31 (s, 3H), 3.79-4.02 (m, 4H), 4.24-4.34 (m,1H), 6.01 (d, 1H, J=15.9 Hz), 6.68 (d,1H, J=8.7 Hz), 6.75 (d,1H, J=16.2 Hz), 6.87-6.98 (m, 4H), 7.29 (d,1H, J=8.7 Hz); MS (ESI+): 482 ([M+NH₄]⁺).

### Example 160

### Preparation of (S)-5-(4-{1-Ethyl-1-[4-((E)-3-ethyl-3-hydroxy-pent-1-enyl)-3-methyl-phenyl)-propyl}-2-methyl-phenoxy)-pentane-1,4-diol

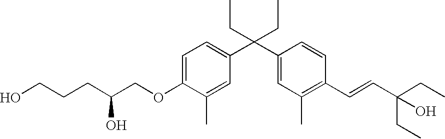

### Preparation of (S)-5-(4-{1-Ethyl-1-[4-((E)-3-ethyl-3-hydroxy-pent-1-enyl)-3-methyl-phenyl]-propyl}-2-methyl-phenoxy)-pentane-1,4-diol

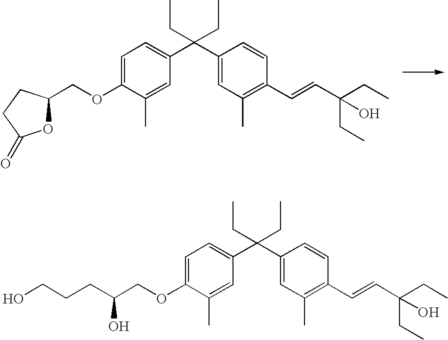

To a solution of (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one (compound prepared in Example 1-(6)) (59.2 mg, 0.124 mmol) in Et₂O (3 ml), LAH (14 mg, 0.371 mmol) was added and the mixture was stirred at room temperature for 18 h. To the mixture, ethyl acetate and brine were added and the organic layer was filtered through celite, and the filtrate was concentrated in vacuo The obtained residue was purified by preparative TLC (n-hexane/ethyl acetate=1/1 and 1/3) to give the title compound (28.8 mg, 48.2%).

¹H-NMR (chloroform-d): 0.61 (t, 6H, J=7.3 Hz), 0.92 (t, 6H, J=7.6 Hz), 1.47 (brs, 1H), 1.64 (q, 4H, J=7.6 Hz), 1.72-1.82 (m, 4H), 2.05 (q, 4H, J=7.3 Hz), 2.18 (s, 3H), 2.31 (s, 3H), 2.81 (brs,1H), 3.70-3.75 (m, 2H), 3.85 (dd, 1H, J=7.5, 9.2 Hz), 3.95 (dd,1H, J=3.5, 9.2 Hz), 4.02-4.09 (m, 1H), 6.01 (d,1H, J=16.0 Hz), 6.68 (d, 1H, J=8.3 Hz), 6.74 (d, 1H, J=16.0 Hz), 6.90-6.97 (m, 4H), 7.30 (d, 1H, J=8.8 Hz); MS (ESI+): 500 ([M+NH₄]⁺).

### Example 161

### Preparation of 4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenol

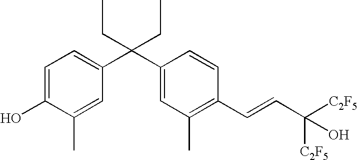

### (1) Preparation of (E)-3-{4-[1-Ethyl-1-(4-hydroxy-3-methyl-phenyl)-propyl]-2-methyl-phenyl}-acrylic acid methyl ester

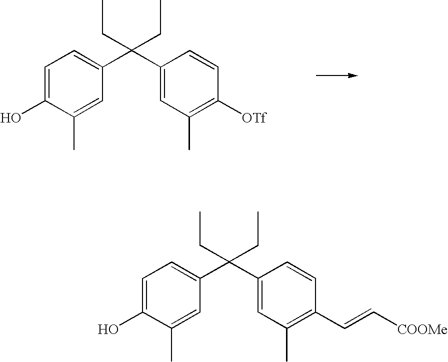

To a solution of compound prepared in Example 1-(1) (trifluoro-methanesulfonic acid 4-[1-ethyl-1-(4-hydroxy-3-methyl-phenyl)-propyl]-2-methyl-phenyl ester), (200 mg, 0.481 mmol) in DMF (2 ml), NaHCO₃ (162 mg, 1.926 mmol), LiBr (29.3 mg, 0.337 mmol), dppp (20 mg, 0.048 mmol), PdCl₂(PPh₃)₂ (33.7 mg, 0.048 mmol) and acrylic acid methyl ester (0.347 ml, 3.85 mmol) were added at room temperature and the mixture was stirred at 140 degrees C. for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=100:0 to 50:50) to give the title compound (15 mg, 9%).

¹H-NMR: 0.61 (t, 6H), 2.03 (q, 4H), 2.20 (s, 3H), 2.39 (s, 3H), 3.80 (s, 3H), 4.67 (s,1H), 6.32 (d,1H), 6.66 (d,1H), 7.42 (d ,1H), 7.96 (d,1H).

### (2) Preparation of 4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenol

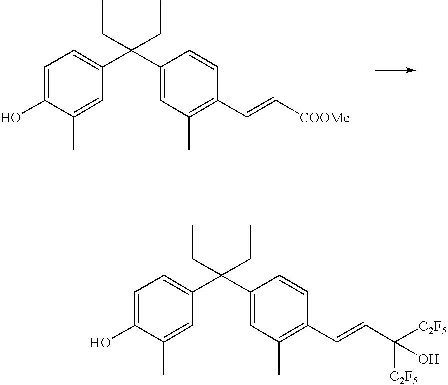

To a solution of C₂F₅1 (0.426 mmol) in diethyl ether (0.5 ml) at −78 degrees C., 1.5M MeLi in diethyl ether (0.284 ml, 0.426 mmol) was added, and then (E)-3-{4-[1-Ethyl-1-(4-hydroxy-3-methyl-phenyl)-propyl]-2-methyl-phenyl}-acrylic acid methyl ester (compound prepared in Example 161-(1)) (15 mg, 0.0426 mmol) in diethyl ether (0.5 ml) were added. The mixture was stirred at −78 degrees C. for 10 min. To the mixture, ethyl acetate was added and the mixture was washed with H₂O, dried over MgSO₄, filtered and concentrated in vacuo. The obtained residue was chromatographed on silica gel (n-hexane:ethyl acetate=100:0 to 50:50) to give the title compound (5 mg, 21%).

¹H-NMR: 0.61 (t, 6H), 2.03 (q, 4H), 2.20 (s, 3H), 2.30 (s, 3H), 3.35 (s, 1H), 5.98 (d, 1H), 6.66 (d, 1H), 6.82-6.90 (m, 2H), 6.95-7.00 (m, 2H), 7.35 (d, 1H); MS (ESI+): 561 ([M+H]⁺).

### Example 162

### Preparation of (S)-5-(4-{1-Ethyl-1-[3-methyl4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one

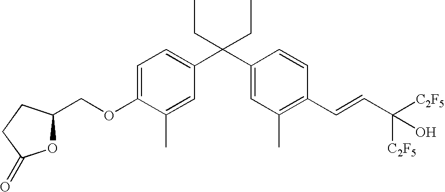

### Preparation of (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one

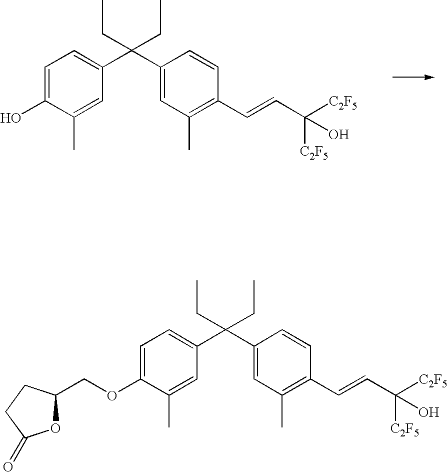

To a solution of compound prepared in Example 161-(2) (4-{1-ethyl-1-[3-methyl-4-((E)-4,4,5,5,5-pentafluoro-3-hydroxy-3-pentafluoroethyl-pent-1-enyl)-phenyl]-propyl}-2-methyl-phenol) (34 mg, 0.0607 mmol) in DMF (0.5 ml), K₂CO₃ (25.3 mg, 0.183 mmol) and (S)-(+)-dihydro-5-(p-tolenesulfonylmethyl)-2(3H)-furanone (18 mg, 0.0668 mmol) were added at room temperature and stirred at 100 degrees C. for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=2:1) to give the title compound (13.7 mg, 34%).

¹H-NMR: 0.61 (t, 6H), 2.05 (q, 4H), 2.20 (s, 3H), 2.34 (s, 3H), 2.55-2.63 (m, 2H), 3.90-3.98 (m, 1H), 4:17-4.22 (m, 1H), 4.634.73 (m ,1H), 5.89 (d, 1H), 6.65 (d, 1H), 6.82-6.90 (m, 2H), 6.97-7.02 (m, 2H), 7.35 (d, 1H); MS (ESI+): 676 ([M+NH₄]⁺).

### Example 163

### Preparation of (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxy)-4-hydroxy-pentanoic acid

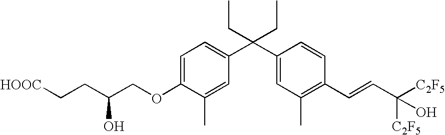

### Preparation of (S)-5-(4-{1-Ethyl-1-[3-methyl-4-((E)A4,4,5,5,5-pentafluoro-3-hydroxy-3-pentafluoroethyl-pent-1-enyl)-phenyl]-propyl}-2-methyl-phenoxy)-4-hydroxy-pentanoic acid

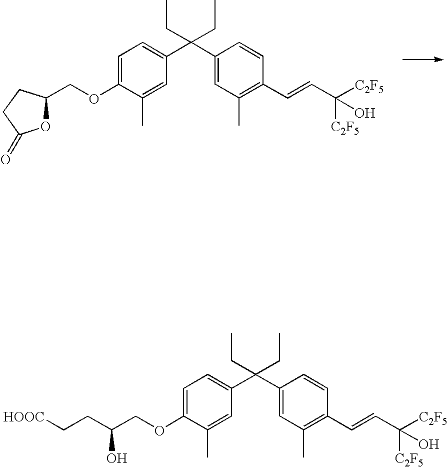

To a solution of compound prepared in Example 162 ((S)-5-(4-{1-Ethyl-1-[3-methyl4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one) (8.1 mg, 0.0123 mmol) in MeOH (1 ml),1 N NaOH (0.1 ml) were added and the mixture was stirred at room temperature for 1 h. The mixture was concentrated in vacuo and purified by preparative TLC (CHCl₃:MeOH=8:3 saturated with H₂O) to give the title compound (4.4 mg, 54%).

¹H-NMR: 0.61 (t, 6H), 2.04 (q, 4H), 2.20 (s, 3H), 2.30 (s, 3H), 2.59 (t, 2H), 3.83-3.98 (m, 3H), 5.92 (d,1H), 6.66 (d, 1H), 6.83-6.89 (m, 2H), 6.97-7.02 (m, 2H); MS (ESI+): 694 ([M+NH₄]⁺).

### Example 164

### Preparation of 4-{1-Ethyl-1-[4-((E)-3-ethyl-3-hydroxy-pent-1-enyl)-3-methyl-phenyl]-propyl}-phenol

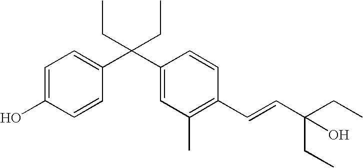

### (1) Preparation of 3-(4-Bromo-3-methyl-phenyl)-pentan-3-ol

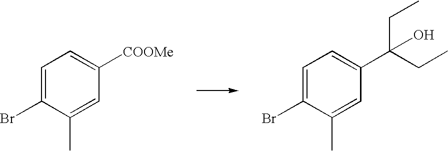

To a solution of 4-bromo-3-methyl-benzoic acid methyl ester (3.0 g, 13.1 mmol) in THF (300 ml), 0.89M EtMgBr in THF (39.2 ml, 34.9 mmol) were added at 0 degrees C. for 5 min and stirred at room temperature for 16 h. To the mixture, saturated NH₄Cl solution and ethyl acetate was added and the organic layer was washed with saturated NH₄Cl solution, dried over MgSO₄, concentrated in vacuo. The obtained residue was chromatographed on silica gel (n-hexane only to ethyl acetate/n-hexane=1/4) to give the title compound (2.71 g, 81%).

¹H-NMR: 0.75 (t, 6H), 1.74-1.82 (m, 4H), 2.40 (s, 3H), 7.00 (dd,1H), 7.45 (d, 1H); MS (ESI+): 239 ([M−H]⁺).

### (2) Preparation of 4-[l-(4-Bromo-3-methyl-phenyl)-1-ethyl-propyl]-phenol

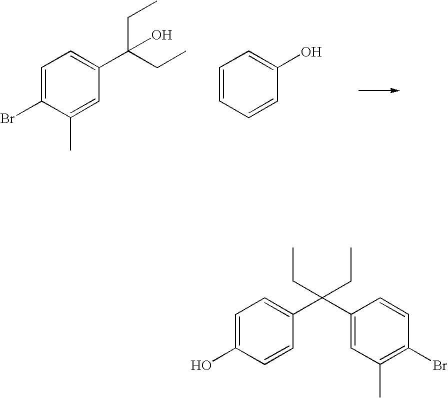

To a mixture of 3-(4-bromo-3-methyl-phenyl)-pentan-3-ol (150 mg, 0.586 mmol) and phenol (82 mg, 0.88 mmol), trifluoromethanesulfonic acid (0.1 ml) were added at room temperature and the mixture was stirred at room temperature for 16 h. The mixture applied onto silica and chromatographed (n-hexane to ethyl acetate/n-hexane=1/1) to give the title compound as mixture of phenol.

¹H-NMR: 0.60 (t, 6H), 2.03 (q, 4H), 2.32 (s, 3H), 6.72 (s,1H), 6.83-7.38 (m, 6H); MS (ESI+): 333 ([M+H]⁺).

### (3) Preparation of (E)-1-{4-[1-Ethyl-1-(4-hydroxy-phenyl)-propyl]-2-methyl-phenyl}-pent-1-en-3-one

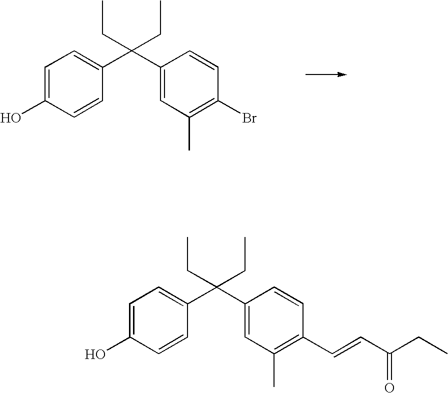

To a solution of 4-[l-(4-Bromo-3-methyl-phenyl)-1-ethyl-propyl]-phenol prepared in Example 164-(2) in DMF (3 ml), NaHCO₃ (454 mg, 5.4 mmol), LiBr (40 mg, 0.46 mmol), dppp (58 mg, 0.14 mmol), PdCl₂(PPh₃)₂ (98 mg, 0.14 mmol) and pent-1-en-3-one (1.08 ml, 1.08 mmol) were added at room temperature and the mixture was stirred at 130 degrees C. for 16 h. To the mixture, ethyl acetate was added and the organic layer was washed with H₂O, dried over MgSO₄, concentrated in vacuo. The obtained residue was purified by preparative TLC (n-hexane:ethyl acetate=1 :3) to give the title compound (353 mg).

¹H-NMR: 0.61 (t, 6H), 1.17 (t, 3H), 2.04 (q, 4H), 2.37 (s, 3H), 2.69 (q, 2H), 6.63 (d, 1H), 6.75 (d, 1H), 6.98-7.05 (m, 2H), 7.45 (d, 1H), 7.82 (d, 1H); MS (ESI+): 337 ([M+H]⁺).

### (4) Preparation of 4-{1-Ethyl-1-[4-((E)-3-ethyl-3-hydroxy-pent-1-enyl)-3-methyl-phenyl]-propyl}-phenol

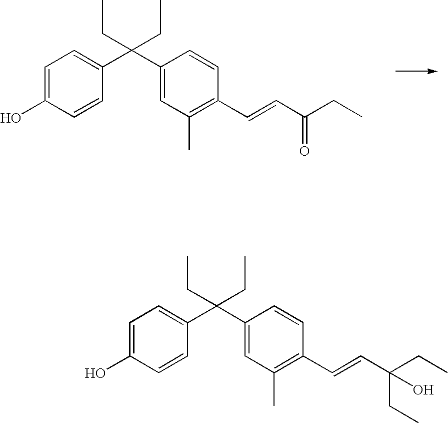

To a solution of (E)-1-{4-[1-ethyl-1-(4-hydroxy-phenyl)-propyl]-2-methyl-phenyl}-pent-1-en-3-one (350 mg, 1.04 mmol) in THF (2 ml), 0.5 M EtLi in THF (1 0.4 ml, 5.21 mmol) was added at 0 degree and the mixture was stirred at room temperature for 1 h. To the mixture, saturated NH₄Cl was added, flirted through celite and concentrated in vacuo. The residue was purified by preparative TLC (ethyl acetate/n-hexane=1/2) to give the title compound (145 mg, 38%).

¹H-NMR: 0.60 (t, 6H), 0.92 (t, 6H), 1.61 (q, 4H), 2.03 (q, 4H), 2.30 (s, 3H), 5.99 (d,1H), 6.70-7.28 (m, 8H); MS (ESI+): 349 ([M−H]⁺).

### Example 165

### Preparation of (S)-5-(4-{1-Ethyl-1-[4-((E)-3-ethyl-3-hydroxy-pent-1-enyl)-3-methyl-phenyl]-propyl}-phenoxy)4-hydroxy-pentanoic acid

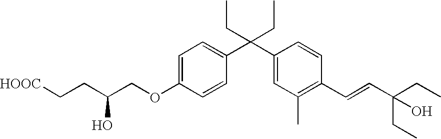

### (1) Preparation of (S)-5-(4-{1-Ethyl-1-[4-((E)-3-ethyl-3-hydroxy-pent-1-enyl)-3-methyl-phenyl]-propyl}-phenoxymethyl)-dihydro-furan-2-one

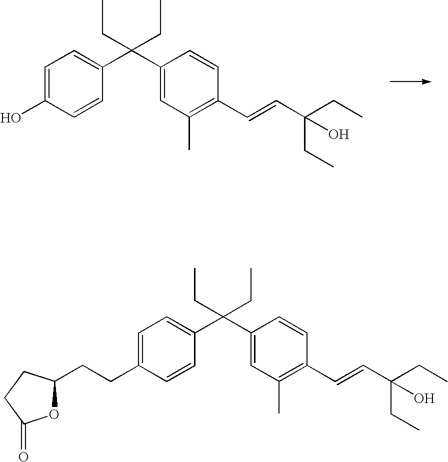

To a solution of 4-{1-ethyl-1-[4-((E)-3-ethyl-3-hydroxy-pent-1-enyl)-3-methyl-phenyl]-propyl}-phenol (compound prepared in Example 164-(4)) (135 mg, 0.369 mmol) in DMF (2 ml), K₂CO₃ (153 mg, 1.11 mmol) and (S)-(+)-dihydro-5-(p-tolenesulfonylmethyl)-2(3H)-furanone (150 mg, 0.554 mmol) were added at room temperature and the mixture was stirred at 100 degrees C. for 16 h. To the mixture, ethyl acetate was added and the mixture was washed with H₂O, dried over MgSO₄, concentrated in vacuo. The obtained residue was chromatographed on silica gel (n-hexane to n-hexane:ethyl acetate=1:1) to give the title compound (117 mg, 68%).

¹H-NMR: 0.62 (t, 6H), 0.91 (t, 6H),. 1.62-1.65 (q, 4H), 2.30 (s, 3H), 4.05-4.17 (m, 2H), 4.82-4.89 (m, 1H), 6.01 (d,1H), 6.70-6.82 (m, 3H), 7.08 (d,1H), 7.29 (d, 1H); MS (ESI+): 465 ([M+H]⁺).

### (2) Preparation of (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid

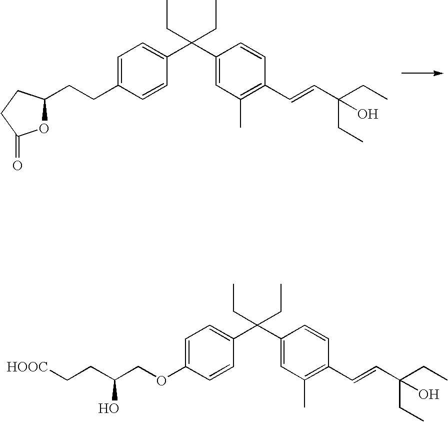

To a solution of (S)-5-(4-{l-Ethyl-1-[4-((E)-3-ethyl-3-hydroxy-pent-1-enyl)-3-methyl-phenyl]-propyl}-phenoxymethyl)-dihydro-furan-2-one (compound prepared in Example 165-(1)) (117 mg, 0.252 mmol) in MeOH (3 ml), 1N NaOH (0.6 ml) were added at room temperature and the mixture was stirred at room temperature for 1 h. The mixture was concentrated in vacuo, and purified by preparative TLC (CHCl₃:MeOH=8:3 saturated with H₂O) to give the title compound (59 mg, 49%).

¹H-NMR: 0.61 (t, 6H), 0.91 (t, 6H), 1.63 (q, 4H), 2.02 (q, 4H), 2.17 (s, 3H), 2.66 (t, 2H), 3.79-3.89 (m, 1H), 3.93-4.08 (m, 2H), 6.00 (d, 1H), 6.72-7.31 (m, 8H); MS (ESI+) 500 ([M+NH₄]⁺).

### Example 166

### Preparation of (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one

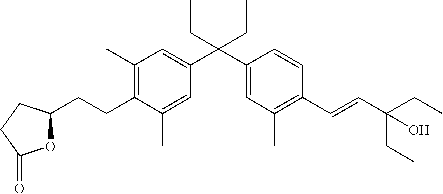

### (1) Preparation of 4-[1-(4-Bromo-3-methyl-phenyl)-1-ethyl-propyl]-2,6-dimethyl-phenol

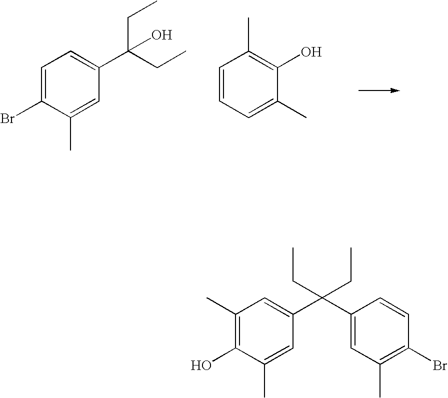

To a solution of 3-(4-Bromo-3-methyl-phenyl)-pentan-3-ol (compound prepared in Example 164-(1)) (200 mg, 0.780 mmol) and 2,6-dimethyl-phenol (285 mg, 2.34 mmol) in toluene (0.2 ml), AlCl₃ (52 mg, 0.390 mmol) was added and the mixture was stirred at room temperature for 60 h. The mixture applied onto silica and chromatographed (n-hexane:CH₂Cl₂=30:70 to 0:100) to give the title compound as mixture of 2,6-dimethyl-phenol (356 mg).

¹H-NMR: 0.59 (t, 6H, —CH₂—CH₃), 2.03 (q, 4H, —CH₂CH₃); MS (ESI+): 361 ([M+H]⁺).

### (2) Preparation of (E)-1-{4-[1-Ethyl-1-(4-hydroxy-3,5-dimethyl-phenyl)-propyl]-2-methyl-phenyl}-pent-1-en-3-one

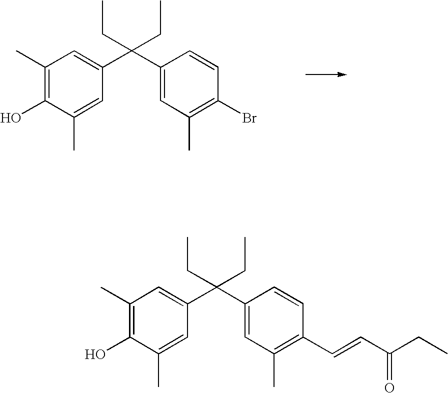

To a solution of 4-[l-(4-bromo-3-methyl-phenyl)-1-ethyl-propyl]-2,6-dimethyl-phenol prepared in Example 166-(1) (110 mg) in DMF (1 ml), NaHCO₃ (81 mg, 0.96 mmol), LiBr (14.7 mg, 0.17 mmol), dppp (10 mg, 0.024 mmol), PdCl₂(PPh₃)₂ (16.9 mg, 0.024 mmol) and pent-1-en-3-one (0.20 ml, 0.20 mmol) were added at room temperature and the mixture was stirred at 140 degrees C. for 16 h. To the mixture, ethyl acetate was added and the mixture was washed with H₂O, dried over MgSO₄, filtered and concentrated in vacuo. The obtained residue was chromatographed on silica gel (CH₂Cl₂:MeOH=100:0 to 10:1) to give the title compound (85 mg).

¹H-NMR: 0.60 (t, 6H), 1.17 (t, 3H), 2.04 (q, 4H), 2.19 (s, 6H), 2.67 (q, 2H), 4.56 (s,1H), 6.65 (d,1H), 6.73 (s,1H), 6.98-7.03 (m, 2H), 7.46 (d,1H), 7.80 (d, 1H); MS (ESI+): 365 ([M+H]⁺).

### (3) Preparation of 4-{1-Ethyl-1-[4-((E)-3-ethyl-3-hydroxy-pent-1-enyl)-3-methyl-phenyl]-propyl}-2,6-dimethyl-phenol

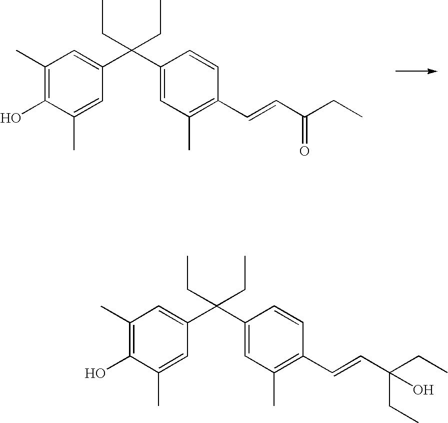

To a solution of (E)-1-{4-[1-Ethyl-1-(4-hydroxy-3,5-dimethyl-phenyl)-propyl]-2-methyl-phenyl}-pent-1-en-3-one (compound prepared in Example 166-(2)) (85 mg, 0.233 mmol) in THF (2 ml), 0.89 M EtMgBr in THF (1.63 ml, 1.35 mmol) was added at 0 degrees C. and the mixture was stirred at room temperature for 1 h. To the mixture, saturated NH₄Cl was added, the mixture was flirted through celite and concentrated in vacuo. The obtained residue was purified by preparative TLC (ethyl acetate/n-hexane=1/4) to give the title compound (17 mg, 19%).

¹H-NMR: 0.60 (t, 6H), 0.92 (t, 6H), 1.61 (q, 4H), 2.02 (q, 4H), 2.19 (s, 6H), 2.38 (s, 3H), 4.47 (s,1H), 6.00 (d,1H), 6.73-7.30 (m, 6H); MS (ESI+): 395 ([M+H]⁺).

### (4) Preparation of (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one

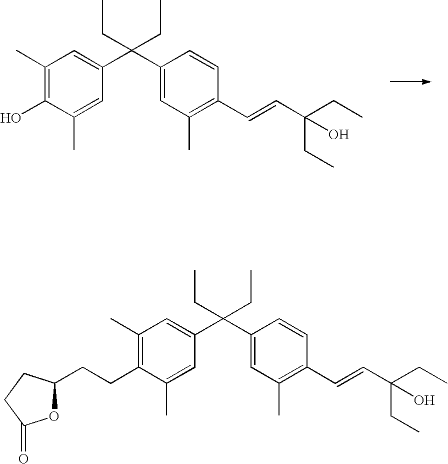

To a solution of 4-{1-Ethyl-1-[4-((E)-3-ethyl-3-hydroxy-pent-1-enyl)-3-methyl-phenyl]-propyl}-2,6-dimethyl-phenol (compound prepared in Example 166-(3)) (16 mg, 0.0406 mmol) in DMF (0.3 ml), K₂CO₃ (16.9 mg, 0.122 mmol) and (S)-(+)-dihydro-5-(p-tolenesulfonylmethyl)-2(3H)-furanone (21.9 mg, 0.0812 mmol) were added at room temperature and the mixture was stirred at 100 degrees C. for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=2:1) to give the title compound (16 mg, 80%).

¹H-NMR: 0.60 (t, 6H), 0.92 (t, 6H), 1.65 (q, 4H), 2.05 (q, 4H), 2.21 (s, 6H), 3.90-4.01 (m, 2H), 4.78-4.82 (m,1H), 6.02 (d,1H), 6.75-7.30 (m, 5H); MS (ESI+): 510 ([M+NH₄]⁺).

### Example 167

### Preparation of (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid

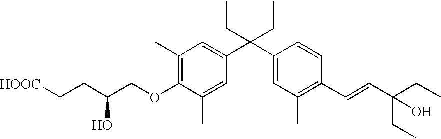

### Preparation of (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid

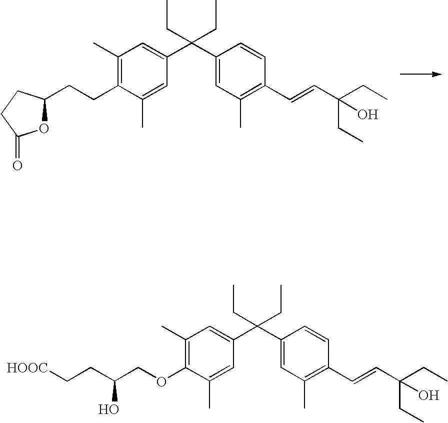

To a solution of (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one (compound prepared in Example 166-(4)) (11 mg, 0.022 mmol) in MeOH (0.3 ml), 1 N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (4.7 mg, 41%).

¹H-NMR: 0.60 (t, 6H), 0.92 (t, 6H), 1.64 (q, 4H), 2.02 (q, 4H), 2.21 (s, 6H), 2.34 (s, 3H), 2.61 (t, 2H), 3.66-3.79 (m, 2H), 4.01-4.10 (m, 1H), 6.02 (d, 1H), 6.72-7.34(m, 6H); MS (ESI+): 528 ([M+NH₄]⁺).

### Example 168

### Preparation of 4-{I-Ethyl-1-[4-((E)-3-ethyl-3-hydroxy-pent-1-enyl)-3-propyl-phenyl]-propyl}-2-methyl-phenol

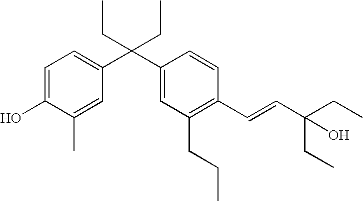

### (1) Preparation of 1-(4-Hydroxy-3-propyl-phenyl)-propan-1-one

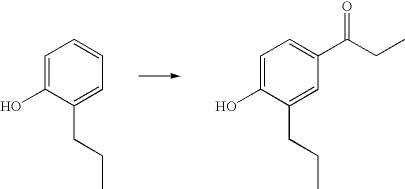

To a mixture of 2-propyl-phenol (2.0 g, 14.7 mmol) and propionyl chloride (1.36 g, 14.7 mmol), AlCl₃ (2.15 g, 16.2 mmol) was added and the mixture was stirred at room temperature for 1 h. The mixture was applied onto silica and chromatographed (ethyl acetate:n-hexane=0:100 to 50:50) to give the title compound (0.30 g, 11%).

¹H-NMR: 0.97 (t, 3H), 1.22 (t, 3H), 1.64 (q, 2H), 2.63 (t, 2H), 2.96 (q, 2H), 6.85 (d, 1H), 7.72-7.79 (m, 2H); MS (ESI+): 193 ([M+H]⁺).

### (2) Preparation of 4-(1-Ethyl-1-hydroxy-propyl)-2-propyl-phenol

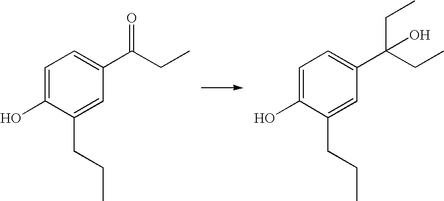

To a solution of 4-(1-ethyl-1-hydroxy-propyl)-2-propyl-phenol (compound prepared in Example 168-(1)) (0.30 g, 1.56 mmol) in THF (5 ml), 3M EtMgBr in diethyl ether (2.6 ml, 7.80 mmol) was added at 0 degrees C. and the mixture was stirred at room temperature for 16 h. To the mixture, H₂O and ethyl acetate were added and the organic layer was washed with H₂O, dried over MgSO₄, concentrated in vacuo. The obtained residue was chromatographed on silica gel (ethyl acetate:n-hexane=0:100 to 50:50) to give the title compound (0.32 g, 94%).

¹H-NMR: 0.76 (t, 6H), 0.96 (t, 3H), 1.77-1.83 (m, 4H), 2.58 (t, 2H), 4.68-4.71 (m, 1H), 6.70 (d, 1H), 7.02-7.07 (m, 2H).

### (3) Preparation of Trifluoro-methanesulfonic acid 4-(1-ethyl-1-hydroxy-propyl)-2-propyl-phenyl ester

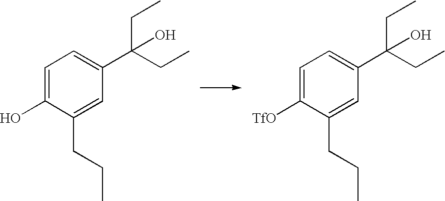

To a solution of 4-(1-ethyl-1-hydroxy-propyl)-2-propyl-phenol (compound prepared in Example 168-(2)) (315 mg, 1.42 mmol) in CH₂Cl₂ (5 ml), pyridine (0.172 ml, 2.13 mmol) and trifluoromethanesulfonic anhydride (0.239 ml, 1.42 mmol) were added at 0 degrees C. and the mixture was stirred at 0 degrees C. for 2 h. The mixture was applied onto silica and chromatographed (ethyl acetate:n-hexane=0:100 to 50:50) to give the title compound (0.21 g, 42%).

¹H-NMR: 0.67 (t, 6H), 0.97 (t, 3H), 1.78-1.83 (m, 4H), 2.65 (t, 2H), 7.16-7.32 (m, 3H).

### (4) Preparation of Trifluoro-methanesulfonic acid 4-[l -ethyl-1-(4-hydroxy-3-methyl-phenyl)-propyl]-2-propyl-phenyl ester

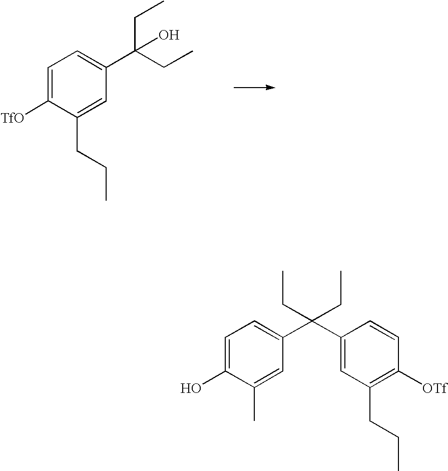

To a mixture of 2-methyl-phenol (84 mg, 0.78 mmol) and trifluoro-methanesulfonic acid 4-(1-ethyl-1-hydroxy-propyl)-2-propyl-phenyl ester (compound prepared in Example 168-(3)) (138 mg, 0.39 mmol) in tolunene (0.3 ml), trifluoromethanesulfonic acid (0.040 ml) was added and the mixture was stirred at room temperature for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=100:0 to 50:50) to give the mixture of the title compound containing 2-methyl-phenol (192 mg).

¹H-NMR: 0.60 (t, 6H, —CH₂CH₃), 0.90 (t, 3H, —CH₂CH₂CH₃),1.65 (q, 2H, —CH₂CH₂CH₃), 2.02 (q, 4H, —CH₂CH₃), 2.12 (s, 3H, Ph-CH₃), 2.61 (t, 2H, —CH₂CH₂CH₃); MS (ESI+): 445 ([M+H]⁺).

### (5) Preparation of (E)-1-{4-[1-Ethyl-1-(4-hydroxy-3-methyl-phenyl)-propyl]-2-propyl-phenyl}-pent-1-en-3-one

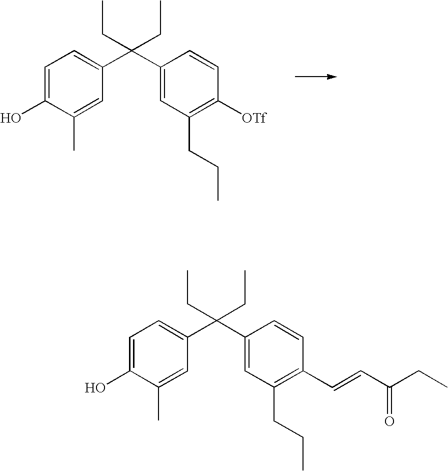

To a solution of compound prepared in Example 168-(4) (trifluoro-methanesulfonic acid 4-[1-ethyl-1-(4-hydroxy-3-methyl-phenyl)-propyl]-2-propyl-phenyl ester) (compound prepared in Example 168-(4)) (192 mg) in DMF (3 ml), NaHCO₃ (131 mg, 1.56 mmol), LiBr (23.4 mg, 0.273 mmol), dppp (16.1 mg, 0.039 mmol), PdCl₂(PPh₃)₂ (27.4 mg, 0.039 mmol) and pent-1-en-3-one (0.381 ml, 3.12 mmol) were added at room temperature and the mixture was stirred at 130 degrees C. for 64 h. To the mixture, ethyl acetate was added and the mixture was washed with H₂O, dried over MgSO₄, filtered and concentrated in vacuo. The obtained residue was purified by preparative TLC (n-hexane:ethyl acetate=3:1) to give the title compound (74 mg).

¹H-NMR: 0.64 (t, 6H), 0.90 (t, 3H), 1.17 (t, 3H), 1.52 (q, 2H), 2.04 (q, 4H), 2.18 (s, 3H), 2.69 (q, 2H), 4.93 (s, 1H), 6.62-6.66 (m, 2H), 6.82-6.86 (m,1H), 7.45 (d, 1H), 7.85 (s, 1H) MS (ESI+): 379 ([M+H]⁺).

### (6) Preparation of 4-{1-Ethyl-1-[4-((E)-3-ethyl-3-hydroxy-pent-1-enyl)-3-propyl-phenyl]-propyl}-2-methyl-phenol

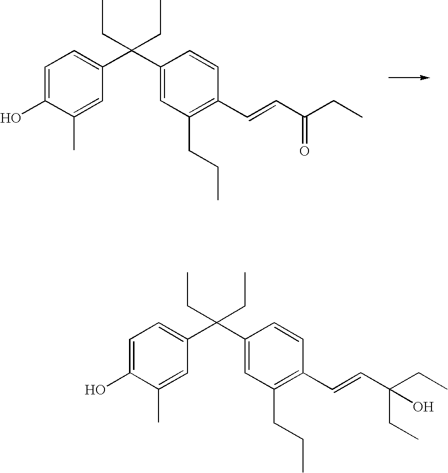

To a solution of (E)-1-{4-[1-Ethyl-1-(4-hydroxy-3-methyl-phenyl)-propyl]-2-propyl-phenyl}-pent-1-en-3-one (compound prepared in Example 168-(5)) (70 mg, 0.185 mmol) in THF (2 ml), 0.5 M EtLi in THF (1.85 ml, 0.93 mmol) was added at 0 degree and the mixture was stirred at room temperature for 16 h. To the mixture, saturated NH₄Cl was added, the products were extracted with ethyl acetate and the extracts were washed with H₂O, dried over MgSO₄, filtered and concentrated in vacuo. The obtained residue was purified by preparative TLC (n-hexane:ethyl acetate=5:1) to give the title compound (34 mg, 45%).

¹H-NMR: 0.61 (t, 6H), 0.92 (t, 6H), 1.62 (q, 4H), 2.04 (q, 4H), 2.20 (s, 3H), 2.61 (t, 2H), 4.78 (s, 1H), 6.00 (d, 1H), 6.62 (d, 1H), 6.73-6.95 (m, 1H), 7.28 (d, 1H); MS (ESI+): 391 ([M−H]⁺).

### Example 169

### Preparation of (S)-5-(4-{l -Ethyl-1-[4-((E)-3-ethyl-3-hydroxy-pent-1-enyl)-3-propyl-phenyl]-propyl}-2-methyl-phenoxymethyl)-dihydro-furan-2-one

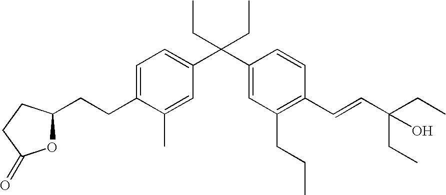

### Preparation of (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one

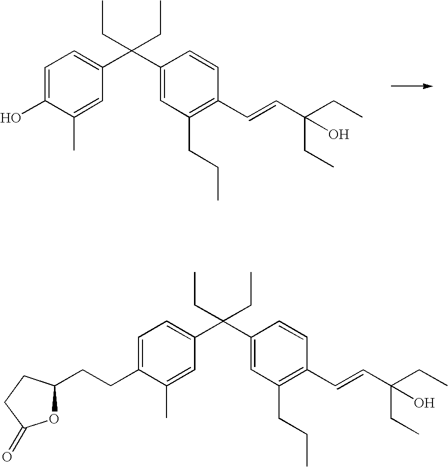

To a solution of 4-{1-Ethyl-1-[4-((E)-3-ethyl-3-hydroxy-pent-1-enyl)-3-propyl-phenyl]-propyl}-2-methyl-phenol (compound prepared in Example 168-(5)) (27 mg, 0.066 mmol) in DMF (0.5 ml), K₂CO₃ (27.3 mg, 0.198 mmol) and (S)-(+)-dihydro-5-(p-toluenesulfonylmethyl)-2(3H)-furanone (26.8 mg, 0.0991 mmol) were added at room temperature and the mixture was stirred at 100 degrees C. for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=2:1) to give the title compound (20 mg, 60%).

¹H-NMR: 0.61 (t, 6H), 0.91 (t, 6H), 1.59 (q, 4H), 2.05 (q, 4H), 2.15 (s, 3H), 2.55 -2.63 (m, 2H), 4.024.20 (m, 2H), 4.83-4.93 (m, 1H), 6.00 (d,1H), 6.63 (d,1H), 6.78 (d, 1H), 7.25 (d, 1H); MS (ESI+): 524 ([M+NH₄]⁺).

### Example 170

### Preparation of (S)-5-(4-{1-Ethyl-1-[4-((E)-3-ethyl-3-hydroxy-pent-1-enyl)-3-propyl-phenyl]-propyl}-2-methyl-phenoxy)4-hydroxy-pentanoic acid

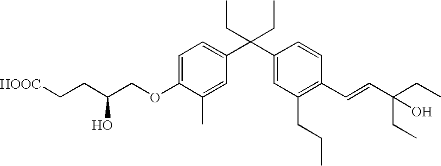

### Preparation of (S)-5-(4-{l-Ethyl-1-[4-((E)-3-ethyl-3-hydroxy-pent-1-enyl)-3-propyl-phenyl]-propyl}-2-methyl-phenoxy)4-hydroxy-pentanoic acid

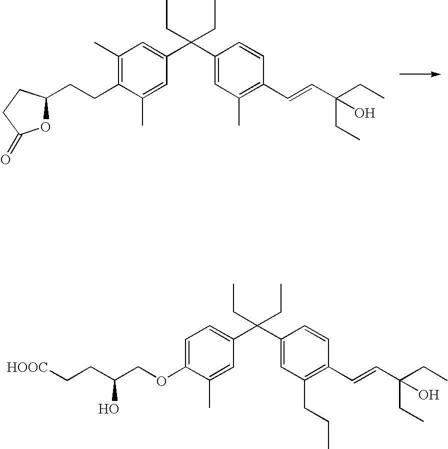

To a solution of (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one (compound prepared in Example 169) (16.8 mg, 0.033 mmol) in THF (1 ml) and MeOH (0.1 ml), 1 N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (12.4 mg, 72%).

¹H-NMR: 0.61 (t, 6H), 0.91 (t, 6H), 1.60 (q, 4H), 2.02 (q, 4H), 2.15 (s, 3H), 3.80-4.15 (m, 3H), 5.98 (d,1H), 6.66 (d, 1H), 6.78 (d, 1H), 6.87-6.93 (m, 2H); MS (ESI+):.507 ([M−H]⁺).

### Example 171

### Preparation of (S)-5-{4-[1-(4-tert-Butylsulfanylmethyl-3-methyl-phenyl)-1-ethyl-propyl]-2-methyl-phenoxymethyl}-dihydro-furan-2-one

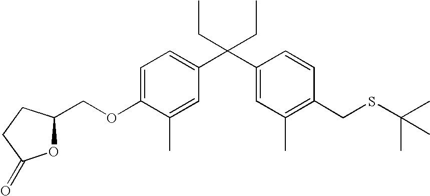

### (1) Preparation of 4-[1-Ethyl-1-(4-hydroxy-3-methyl-phenyl)-propyl]-2-methyl-benzoic acid methyl ester

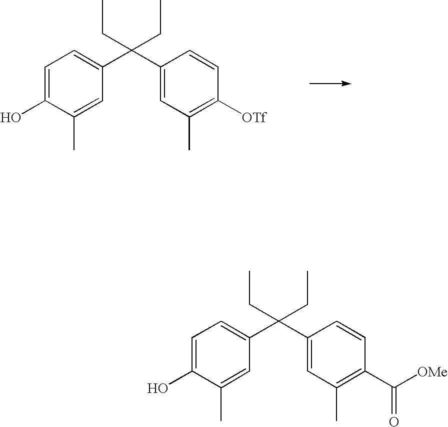

To a solution of trifluoro-methanesulfonic acid 4-[1-ethyl-1-(4-hydroxy-3-methyl-phenyl)-propyl]-2-methyl-phenyl ester (compound prepared in Example 1-(1)) (1480 mg, 3.554 mmol) in DMF (25 ml) and MeOH (25 ml), Pd(OAc)₂ (240 mg, 1.069 mmol), dppp (440 mg, 1.067 mmol), and Et₃N (1.3 ml, 9.321 mmol) were added at room temperature and the mixture was stirred at 100 degrees C. for 16 h under CO gas. To the mixture, ethyl acetate was added and the mixture was washed with saturated ammonium chloride solution and brine, dried over magnesium sulfate, filtered and concentrated in vacuo. The obtained residue was chromatographed on silica gel (n-hexane/ethyl acetate=10/1 to 3/1) to give the title compound (363 mg, 31.3%).

¹H-NMR (chloroform-d): 0.61 (t, 6H, J=7.2 Hz), 2.07 (q, 4H, J=7.2 Hz), 2.19 (s, 3H), 2.56 (s, 3H), 3.87 (s, 3H), 4.88 (brs,1H), 6.66 (d,1H, J=8.1 Hz), 6.82-6.86 (m, 2H), 7.03-7.06 (m, 2H), 7.80 (d,1H, J=9.0 Hz); MS (ESI+): 349 ([M+Na]⁺).

### 5 (2) Preparation of 4-{1-Ethyl-1-[3-methyl-4-(tetrahydro-pyran-2-yloxy)-phenyl]-propyl}-2-methyl-benzoic acid methyl ester

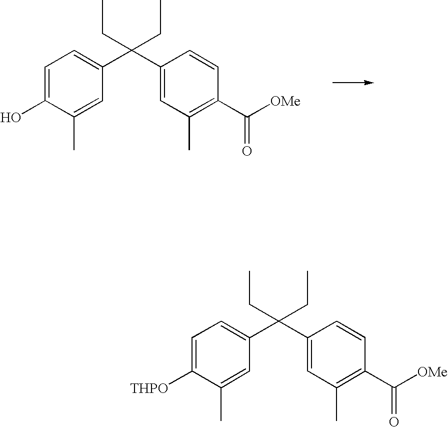

To a solution of 4-[l -Ethyl-1-(4-hydroxy-3-methyl-phenyl)-propyl]-2-methyl-benzoic acid methyl ester (compound prepared in Example 171-(1)) (220 mg, 0.674 mmol) in dichloromethane (1 ml), DHP (0.075 ml, 0.809 mmol) and PPTs (17 mg, 0.068 mmol) were added and the mixture was stirred at room temperature for 25.3 h. To the mixture, ethyl acetate was added and the mixture was washed with brine, dried over magnesium sulfate, filtered and concentrated in vacuo. The obtained residue was chromatographed on silica gel (n-hexane/ethyl acetate=10/1 to 3/1) to give the title compound (243 mg, 87.8%)

¹H-NMR (chloroform-d): 0.63 (t, 6H, J=7.3 Hz), 1.55-1.76 (m, 3H), 1.85-1.88 (m, 2H), 1.95-2.13 (m, 1H), 2.09 (q, 4H, J=7.3 Hz), 2.21 (s, 3H), 2.58 (s, 3H),3.59-3.63 (m, 1H), 3.87 (s, 3H), 3.91-3.98 (m, 1H), 5.39 (t, 1H, J=3.2 Hz), 6.88-7.13 (m, 5H), 7.82 (d, 1H, J=8.8 Hz); MS (ESI+): 433 ([M+Na]⁺).

### (3) Preparation of (4-{1-Ethyl-1-[3-methyl-4-(tetrahydro-pyran-2-yloxy)-phenyl]-propyl}-2-methyl-phenyl)-methanol

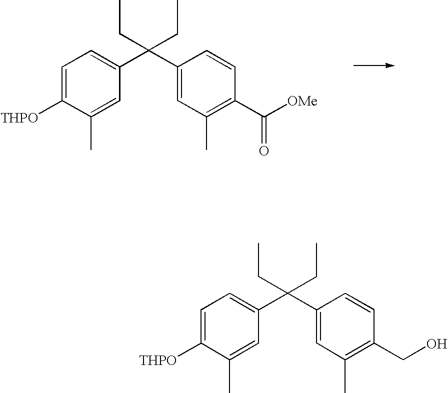

To a solution of 4-{1-Ethyl-1-[3-methyl-4-(tetrahydro-pyran-2-yloxy)-phenyl]-propyl}-2-methyl-benzoic acid methyl ester (compound prepared in Example 171-(2)) (177 mg, 0.431 mmol) in THF (2 ml), LAH (20 mg, 0.527 mmol) was added and the mixture was stirred at room temperature for 18 h. To the mixture, ethyl acetate and brine were added and the organic layer was filtered through celite, concentrated in vacuo. The obtained residue was chromatographed on silica gel (n-hexane/ethyl acetate=1/1) to give the title compound (165 mg, quant.).

1H-NMR (chloroform-d): 0.61 (t, 6H, J=7.3 Hz), 1.58-1.74 (m, 4H), 1.84-1.89 (m, 2H), 2.06 (q, 4H, J=7.3 Hz), 2.20 (s, 3H), 2.30 (s, 3H), 3.58-3.64 (m, 1H), 3.90-3.98 (m, 1H), 4.66 (s, 2H), 5.37 (t,1H, J=3.3 Hz), 6.89-7.03 (m, 5H), 7.20 (d, 1H, J=7.9 Hz).

### (4) Preparation of 2-{4-[1-(4-Chloromethyl-3-methyl-phenyl)-1-ethyl-propyl]-2-methyl-phenoxy}-tetrahydro-pyran

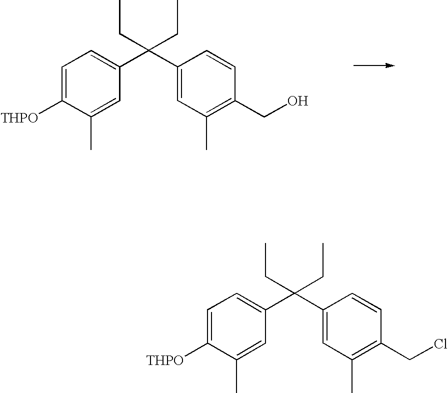

To a solution of (4-{1-Ethyl-1-[3-methyl4-(tetrahydro-pyran-2-yloxy)-phenyl]-propyl}-2-methyl-phenyl)-methanol (compound prepared in Example 171-(3)) (178.3 mg, 0.466 mmol) in CH₂Cl₂ (3 ml), MsCl (0.1 ml, 1.292 mmol), Et₃N (325 ml, 2.33 mmol) and DMAP (6 mg, 0.049 mmol) were added at 0 degrees C. and the mixture was stirred at 0 degrees C. for 17 h. To the mixture, ethyl acetate was added and the mixture was washed with saturated sodium hydrogen carbonate solution and brine, dried over magnesium sulfate, filtered and concentrated in vacuo. To the solution of the residue in DMF (1 ml), LiCl (20 mg, 0.472 mmol) was added and the mixture was stirred at room temperature for 2.5 h. To the mixture, ethyl acetate was added and the mixturer was washed with saturated ammonium chloride solution and brine, dried over magnesium sulfate, filtered and concentrated in vacuo. The obtained residue was chromatographed on silica gel (n-hexane/ethyl acetate=5/1) to give the title compound (137.9 mg, 73.8%).

¹H-NMR (chloroform-d): 0.61 (t, 6H, J=7.3 Hz), 1.57-1.75 (m, 4H), 1.85-1.90 (m, 2H), 0.00 (q, 4H, J=7.3 Hz), 2.21 (s, 3H), 2.37 (s, 3H), 3.58-3.65 (m,1H), 3.90-3.98 (m, 1H), 4.60 (s, 2H), 5.38 (t, 1H, J=3.3 Hz), 6.89-7.00 (m, 5H), 7.18 (d, 1H, J=8.8 Hz); MS (ESI+): 418 ([M+NH₄]⁺).

### (5) Preparation of 2-{4-[1-(4-tert-Butylsulfanylmethyl-3-methyl-phenyl)-1-ethyl-propyl]-2-methyl-phenoxy}-tetrahydro-pyran

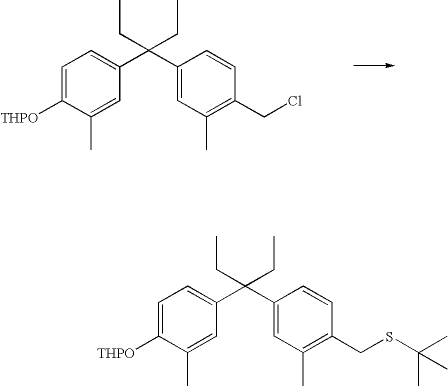

To a solution of 2-{4-[1-(4-Chloromethyl-3-methyl-phenyl)-1-ethyl-propyl]-2-methyl-phenoxy}-tetrahydro-pyran (compound prepared in Example 171-(4)) (96.2 mg, 0.24 mmol) in Acetone (3 ml), t-BuSH (334 mg, 3.71 mmol), KOH (212 mg, 3.79 mmol) in EtOH (6 ml) was added at room temperature and the mixture was stirred at 71 degrees C. for 1 h. To the mixture, ethyl acetate was added and the mixture was washed with saturated sodium hydrogen carbonate solution and brine, dried over magnesium sulfate, filtered and concentrated in vacuo. The obtained residue was chromatographed on silica gel (n-hexane/ethyl acetate=7/1 to 3/1) to give the title compound (106.2 mg, 97.4%).

¹H-NMR (chloroform-d): 0.60 (t, 6H, J=7.3 Hz), 1.40 (s, 9H), 1.55-1.75 (m, 4H), 1.84-1.90 (m, 2H), 2.03 (q, 4H, J=7.3 Hz), 2.20 (s, 3H), 2.36 (s, 3H), 3.55-3.65 (m, 1H), 3.73 (s, 2H), 3.91-3.98 (m, 1H), 5.37 (t, 1H, J=3.2 Hz), 6.89-6.99 (m, 5H), 7.13 (d, 2H, J=8.5 Hz); MS (ESI+): 472([M+NH₄]⁺).

### (6) Preparation of (S)-5-{4-[1-(4-tert-Butylsulfanylmethyl-3-methyl-phenyl)-1-ethyl-propyl]-2-methyl-phenoxymethyl}-dihydro-furan-2-one

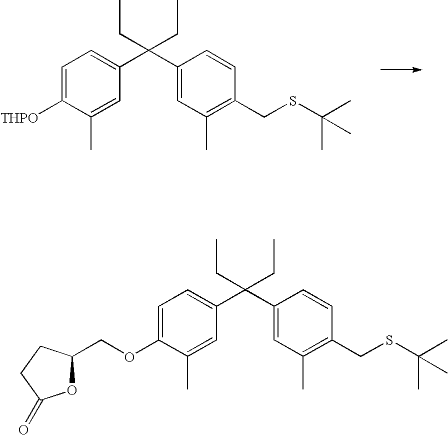

To a solution of 2-{4-[1-(4-tert-Butylsulfanylmethyl-3-methyl-phenyl)-1-ethyl-propyl]-2-methyl-phenoxy}-tetrahydro-pyran (compound prepared in Example 171-(5)) (106.2 mg, 0.234 mmol) in MeOH (3 ml), Montmorillinite K-10 (60 mg) was added and the mixture was stirred at room temperature for 17.5 h. To the mixture, ethyl acetate and brine were added and the organic layer was filtered through celite, concentrated in vacuo. To the solution of the residue in DMF (3 ml), toluene4-sulfonic acid (S)-5-oxo-tetrahydro-furan-2-ylmethyl ester (140 mg, 0.515 mmol), K₂CO₃ (75 mg, 0.538 mmol) were added at room temperature and the mixture was stirred at 83 degrees C. for 8 h. To the mixture, ethyl acetate was added and the mixture was washed with brine, dried over magnesium sulfate, filtered and concentrated in vacuo. The obtained residue was chromatographed on silica gel (n-hexane/ethyl acetate=3/1) to give the title compound (71.2 mg, 64.9%) as colorless oil.

¹H-NMR (chloroform-d): 0.58 (t, 6H, J=7.3 Hz), 1.39 (s, 9H), 2.02 (q, 4H, J=7.3 Hz), 2.15 (s, 3H), 2.35 (s, 3H), 2.27-2.62 (m, 3H), 2.77 (ddd, 1H, J=7.3, 10.2, 17.6 Hz), 3.72 (s, 2H), 4.06 (dd, 1H, J=3.5, 10.4 Hz), 4.16 (dd, 1H, J=3.5, 10.4 Hz), 4.84-4.91 (m, 1H), 6.65 (d, 1H, J=8.2 Hz), 6.90-6.95 (m, 4H), 7.12 (d, 1H, J=8.5 Hz); MS (ESI+):486 ([M+NH₄]⁺).

### Example 172

### Preparation of (S)-5-{4-[1-(4-tert-Butylsulfanylmethyl-3-methyl-phenyl)-1-ethyl-propyl]-2-methyl-phenoxy}4-hydroxy-pentanoic acid

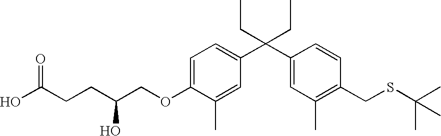

### Preparation of (S)-5-{4-[1-(4-tert-Butylsulfanylmethyl-3-methyl-phenyl)-1-ethyl-propyl]-2-methyl-phenoxy}4-hydroxy-pentanoic acid

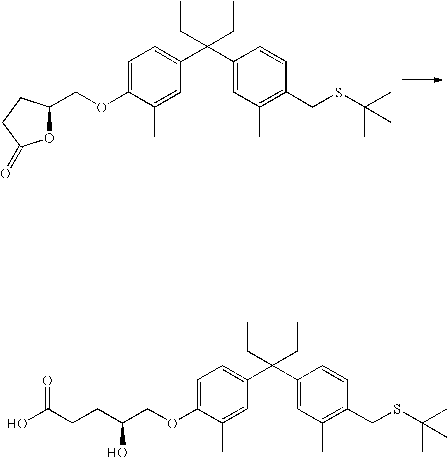

To a solution of (S)-5-{4-[1-(4-tert-Butylsulfanylmethyl-3-methyl-phenyl)-1-ethyl-propyl]-2-methyl-phenoxymethyl}-dihydro-furan-2-one (compound prepared in Example 171) (10 mg, 0.021 mmol) in MeOH (1 ml),1 N KOH aqueous solution (0.12 ml, 0.12 mmol) was added and stirred at room temperature for 12 h. The mixture was concentrated in vacuo and purified by preparative TLC (chroloform/methanol=8/3, saturated by water) to give the title compound (5.5 mg, 53%).

¹H-NMR (chloroform-d): 0.60 (t, 3H, J=7.5 Hz), 1.39 (s, 9H), 1.88-2.12 (m, 2H), 2.04 (q, 4H, J=7.5 Hz), 2.18 (s, 3H), 2.36 (s, 3H), 2.63 (brt, 2H, J=7.2 Hz), 3.73 (s, 2H), 3.86 (dd, 1H, J=6.8, 9.2 Hz), 3.99 (dd, 1H, J=3.5, 9.2 Hz), 4.05-4.13 (m, 1H), 6.68 (d, 1H, J=8.3 Hz), 6.92-6.96 (m, 4H), 7.14 (d, 1H, J=8.5 Hz); MS (ESI+): 504 ([M+NH₄]⁺).

### Example 173

### Preparation of 5(S)-(4-{1-Ethyl-1-[3-methyl4-(2-methyl-propane-2-sulfinylmethyl)-phenyl]-propyl}-2-methyl-phenoxymethyl)-dihydro-furan-2-one

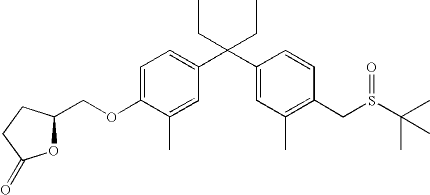

### Preparation of 5(S)-(4-{1-Ethyl-1-[3-methyl4-(2-methyl-propane-2-sulfinylmethyl)-phenyl]-propyl}-2-methyl-phenoxymethyl)-dihydro-furan-2-one

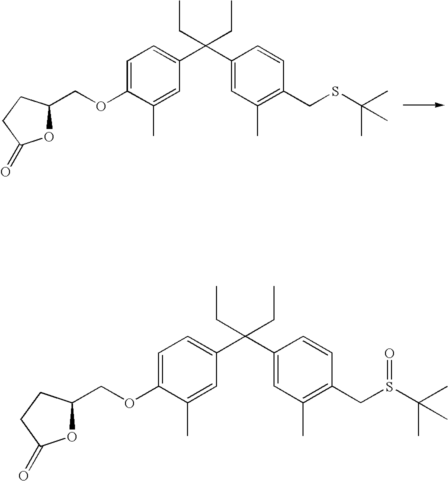

To a solution of (S)-5-{4-[I-(4-tert-Butylsulfanylmethyl-3-methyl-phenyl)-1-ethyl-propyl]-2-methyl-phenoxymethyl}-dihydro-furan-2-one (compound prepared in Example 171) (20 mg, 0.043 mmol) in CH₂Cl₂ (6 ml), mCPBA (>65%) (10.75 mg, 0.041 mmol) in CH₂Cl₂ (2 ml) was added slowly at -78 degrees C. and the mixture was stirred at −78 degrees C. for 7 min. The mixture was concentrated in vacuo and purified by preparative TLC (n-hexane/ethyl acetate=1/99) to give the title compound (18.7 mg, 90.4%).

¹H-NMR (chloroform-d): 0.59 (t, 6H, J=7.3 Hz), 1.34 (s, 9H), 2.04 (q, 4H, J=7.3 Hz), 2.16 (s, 3H), 2.30 (s, 3H), 2.26-2.62 (m, 3H), 2.77 (ddd, 1H, J=7.1, 10.3, 17.2 Hz), 3.59 (d, 1H, J=12.9 Hz), 3.88 (d, 1H, J=12.7 Hz), 4.04-4.19 (m, 2H), 4.85-4.91 (m,1H), 6.66 (d,1H, J=8.3 Hz), 6.91-7.00 (m, 4H), 7.15 (d,1H, J=7.9 Hz); MS (ESI+): 485 ([M+H]⁺).

### Example 174

### Preparation of 5-(4-{1-Ethyl-1-[3-methyl-4-(2-methyl-propane-2-sulfinylmethyl)-phenyl]-propyl}-2-methyl-phenoxy)4(S)-hydroxy-pentanoic acid

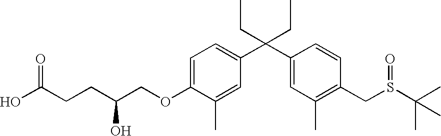

### Preparation of 5-(4-{1-Ethyl-1-[3-methyl-4-(2-methyl-propane-2-sulfinylmethyl)-phenyl]-propyl}-2-methyl-phenoxy)-4(S)-hydroxy-pentanoic acid

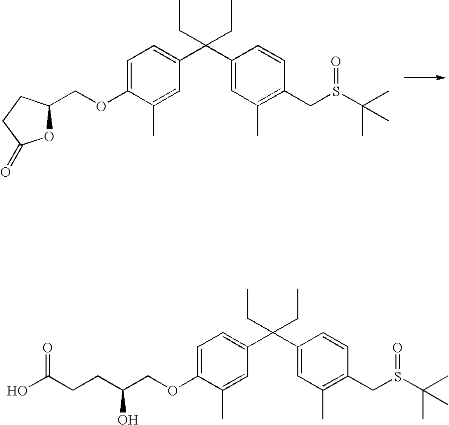

To a solution of 5(S)-(4-{1-Ethyl-1-[3-methyl-4-(2-methyl-propane-2-sulfinylmethyl)-phenyl]-propyl}-2-methyl-phenoxymethyl)-dihydro-furan-2-one (compound prepared in Example 173) (10 mg, 0.021 mmol) in MeOH (1 ml),1 N KOH aqueous solution (0.12 ml, 0.124 mmol) was added and the mixture was stirred at room temperature for 12 h. The mixture was concentrated in vacuo and purified by preparative TLC (chroloform/methanol=8/3, saturated by water) to give the title compound (2.3 mg, 22.2%).

¹H-NMR (chloroform-d): 0.60 (t, 6H, J=7.3 Hz), 1.34 (s, 9H), 1.85-1.98 (m, 2H), 2.05 (q, 4H, J=7.3 Hz), 2.18 (s, 3H), 2.31 (s, 3H), 2.59 (t, 2H, J=7.2 Hz), 3.60 (d, 1H, J=13.0 Hz), 3.89 (d, 1H, J=13.2 Hz), 3.83-4.00 (m, 2H), 4.03-4.09 (m,1H), 6.68 (d, 1H, J=8.8 Hz), 6.91-6.98 (m, 4H), 7.15 (d, 1H, J=8.3 Hz); MS (ESI+): 503 ([M+H]⁺).

### Example 175

### Preparation of (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one

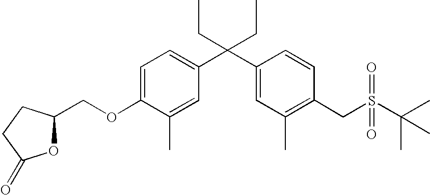

### Preparation of (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one

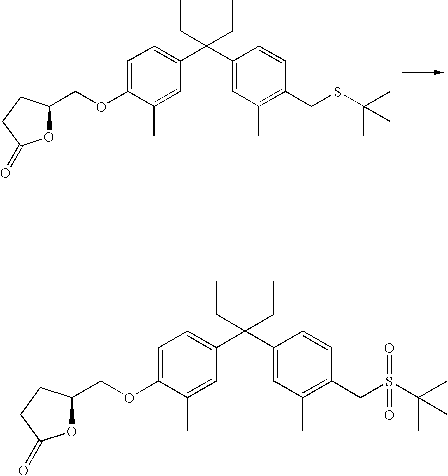

To a solution of (S)-5-{4-[1-(4-tert-Butylsulfanylmethyl-3-methyl-phenyl)-1-ethyl-propyl]-2-methyl-phenoxymethyl}-dihydro-furan-2-one (compound prepared in Example 171) (20 mg, 0.043 mmol) in CH₂Cl₂ (6 ml), mCPBA (>65%) (20 mg 0.085 mmol) in CH₂Cl₂ (2 ml) was added slowly at 0 degrees C. and the mixture was stirred at 0 degrees C. for 0.5 h. The mixture was concentrated in vacuo and purified by preparative TLC (n-hexane/ethyl acetate=3/1) to give the title compound (20.3 mg, 95%).

¹H-NMR (chloroform-d): 0.59 (t, 6H, J=7.4 Hz), 1.49 (s, 9H), 2.04 (q, 4H, J=7.4 Hz), 2.16 (s, 3H), 2.37 (s, 3H), 2.26-2.62 (m, 3H), 2.77 (ddd, 1H, J=7.0, 10.2, 17.2 Hz), 4.04-4.20 (m, 2H), 4.20 (s, 2H), 4.85-4.91 (m,1H), 6.66 (d,1H, J=8.2 Hz), 6.92-7.01 (m, 4H), 7.23 (d, 1H, J=8.5 Hz); MS (ESI+): 518 ([M+NH₄]⁺).

### Example 176

### Preparation of (S)-5-(4-{1-Ethyl-1-[3-methyl-4-(2-methyl-propane-2-sulfonylmethyl)-phenyl]-propyl}-2-methyl-phenoxy)-4-hydroxy-pentanoic acid

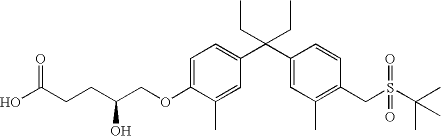

### Preparation of (S)-5-(4-{1-Ethyl-1-[3-methyl-4-(2-methyl-propane-2-sulfonylmethyl)-phenyl]-propyl}-2-methyl-phenoxy)4-hydroxy-pentanoic acid

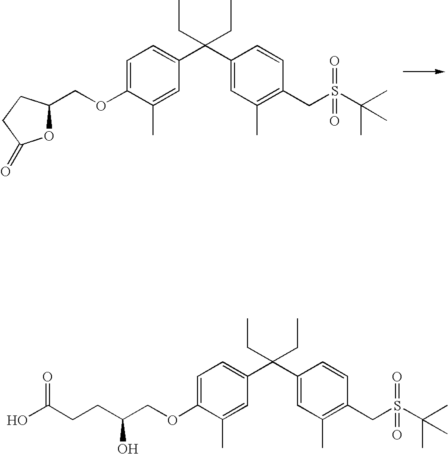

To a solution of (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one (compound prepared in Example 175) (10 mg, 0.02 mmol) in MeOH (1 ml), 1 N KOH aqueous solution (0.12 ml, 0.12 mmol was added and the mixture was stirred at room temperature for 12 h. The mixture was concentrated in vacuo and purified by preparative TLC (chroloform/methanol=8/3, saturated by water) to give the title compound (4.3 mg, 41.5%).

¹H-NMR (chloroform-d): 0.60 (t, 6H, J=7.3 Hz), 1.49 (s, 9H), 1.83-2.14 (m, 2H), 0.00 (q, 4H, J=7.3 Hz), 2.17 (s, 3H), 2.37 (s, 3H), 2.62 (t, 2H, J=7.5 Hz), 3.85 (dd, 1H, J=6.8, 9.3 Hz), 3.97 (dd,1H, J=3.6, 9.3 Hz), 4.04-4.11 (m,1H), 4.20 (s, 2H), 6.68 (d,1H, J=8.3 Hz), 6.91-7.02 (m, 4H), 7.23 (d,1H, J=8.5 Hz); MS (ESI+): 536 ([M+NH₄]⁺).

### Example 177

### Preparation of 5(S)-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one

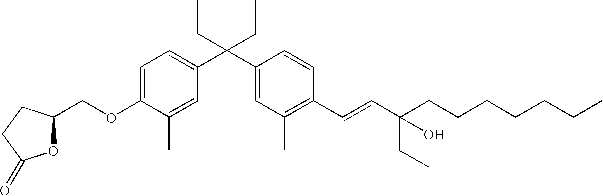

### (1) Preparation of (E)-1-{4-[1-Ethyl-1-(4-hydroxy-3-methyl-phenyl)-propyl]-2-methyl-phenyl}-pent-1-en-3-one

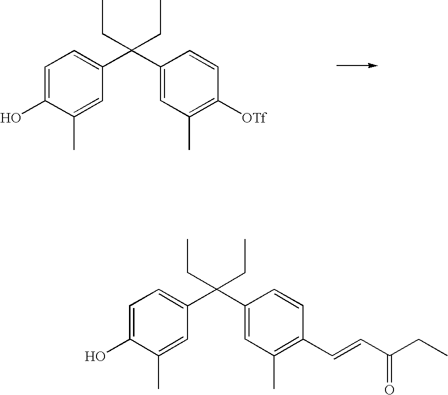

To a solution of compound prepared in Example 1-(1) (trifluoro-methanesulfonic acid 4-[1-ethyl-1-(4-hydroxy-3-methyl-phenyl)-propyl]-2-methyl-phenyl ester) (100 mg, 0.24 mmol) in DMF (1 ml), NaHCO₃ (80.6 mg, 0.96 mmol), LiBr (14.6 mg, 0.17 mmol), dppp (9.9 mg, 0.024 mmol), PdCl₂(PPh₃)₂ (16.8 mg, 0.024 mmol) and acrylic acid methyl ester (0.20 ml, 2.0 mmol) were added at room temperature and the mixture was stirred at 150 degrees C. for 5 h. To the mixture, ethyl acetate was added and the mixture was washed with H₂O, dried over MgSO₄, filtered and concentrated in vacuo. The obtained residue was chromatographed on silica gel (n-hexane:ethyl acetate=1 00:0 to 70:30) to give the title compound (33 mg, 39%).

¹H-NMR: 0.61 (t, 6H), 1.13 (t, 3H), 2.02 (q, 4H), 2.15 (s, 3H), 2.37 (s, 3H), 3.75 (s,1H), 6.58 (m, 2H), 6.78-6.83 (m, 2H), 6.97-7.02 (m, 2H), 7.45 (d,1H), 7.81 (d, 1H); MS (ESI+): 351 ([M+H]⁺).

### (2) Preparation of 4-{1-Ethyl-1-[4-((E)-3-ethyl-3-hydroxy-dec-1-enyl)-3-methyl-phenyl]-propyl}-2-methyl-phenol

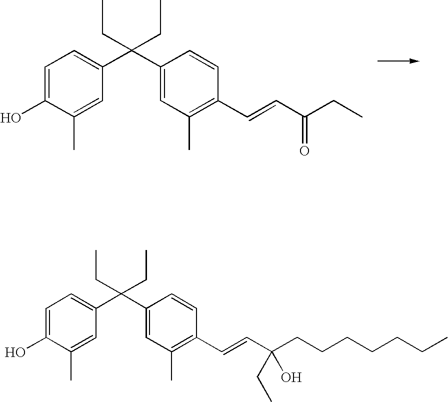

To a solution of (E)-1-{4-[1-ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 1M heptyl magnesium bromide in THF (0.80 ml, 0.80 mmol) was added at 0 degrees C. and the mixture was stirred at 0 degrees C. for 30 min and at room temperature for 2 h. To the mixture, saturated NH₄Cl solution was added and the mixture was filtrated through celite, concentrated in vacuo. The obtained residue was chromatographed on silica gel (n-hexane:ethyl acetate=100:0 to 50:50) to give the title compound (17 mg, 19%).

¹H-NMR: 0.61 (t, 6H), 0.83-0.94 (m, 6H), 2.02 (q, 4H), 2.19 (s, 3H), 2.29 (s,3H), 4.73 (s, 1H), 6.02 (d, 1H), 6.62-7.31 (m, 7H); MS (ESI+): 433 ([M−H]⁺).

### (3) Preparation of 5(S)-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one

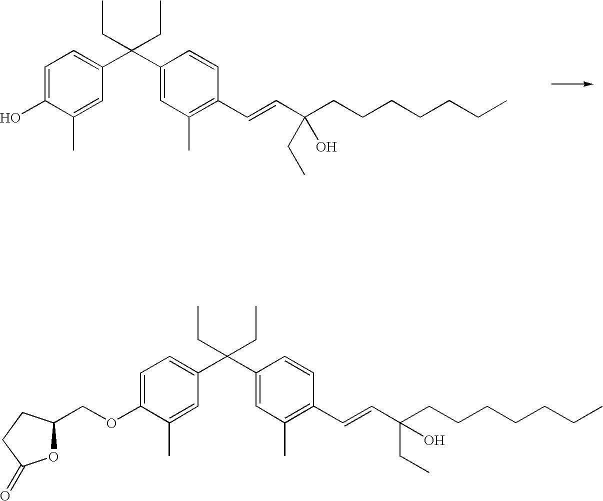

To a solution of 4-{1-Ethyl-1-[4-((E)-3-ethyl-3-hydroxy-dec-1-enyl)-3-methyl-phenyl]-propyl}-2-methyl-phenol (compound prepared in Example 177-(2)) (17 mg, 0.0378 mmol) in DMF (0.3 ml), K₂CO₃ (15.7 mg, 0.113 mmol) and (S)-(+)-dihydro-5-(p-toluenesulfonylmethyl)-2(3H)-furanone (20.4 mg, 0.0756 mmol) were added at room temperature and the mixture was stirred at 110 degrees C. for 16 h. To the mixture, ethyl acetate was added and the mixture was washed with H₂O, dried over MgSO₄, filtered and concentrated in vacuo. The obtained residue was chromatographed on silica gel (n-hexane:ethyl acetate=100:0 to 5:1) to give the title compound (10.5 mg, 51%).

¹H-NMR: 0.60 (t, 6H), 0.82-0.92 (m, 6H), 2.02 (q, 4H), 2.17 (s, 3H), 2.32 (s, 3H), 4.02-4.18 (m, 2H), 4.84-4.91 (m, 1H), 6.01 (d, 1H), 6.63-6.75 (m, 2H), 6.91-6.98 (m, 4H), 7.29 (d,1H); MS (ESI+):566 ([M+NH₄]⁺).

### Example 178

### Preparation of 5(S)-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one

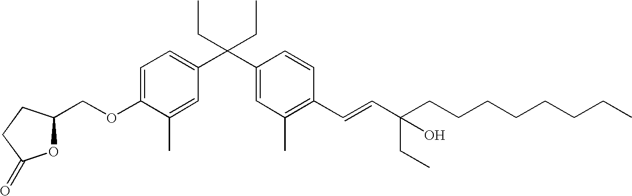

### (1) Preparation of 4-{1-Ethyl-1-[4-((E)-3-ethyl-3-hydroxy-undec-1-enyl)-3-methyl-phenyl]-propyl}-2-methyl-phenol

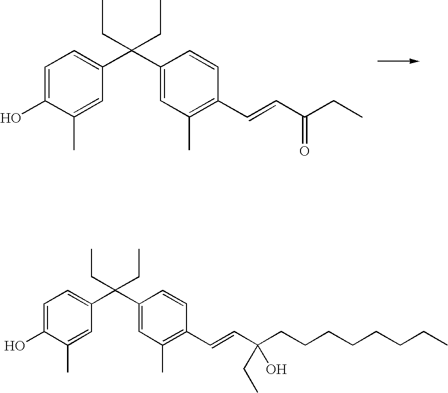

To a solution of (E)-1-{4-[1-ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 2M octyl magnesium bromide in THF (0.40 ml, 0.80 mmol) were added at 0 degrees C. and the mixture was stirred at 0 degrees C. for 30 min and at room temperature for 2 h. To the mixture, saturated NH₄Cl solution was added and the mixture was filtrated through celite and concentrated in vacuo. The obtained residue was chromatographed on silica gel (n-hexane:ethyl acetate=100:0 to 50:50) to give the title compound (22 mg, 24%).

¹H-NMR: 0.60 (t, 6H), 0.80-0.90 (m, 6H), 2.02 (q, 4H), 2.19 (s, 3H), 2.29 (s, 3H), 6.02 (d,1H), 6.61-7.30 (m, 7H); MS (ESI+): 447 ([M−OH]⁺).

### (2) Preparation of 5(S)-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one

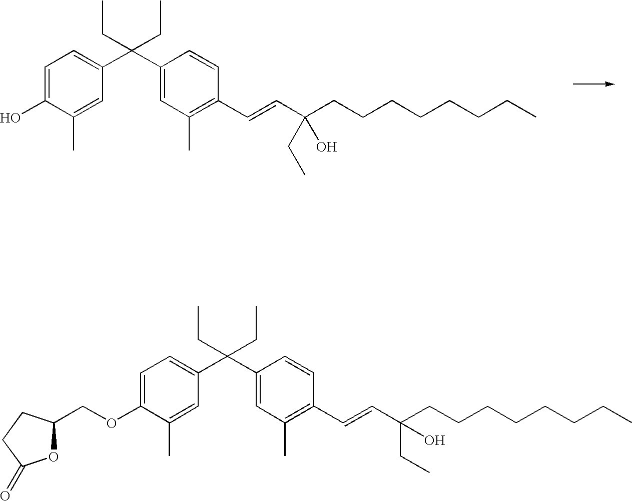

To a solution of 4-{1-Ethyl-1-[4-((E)-3-ethyl-3-hydroxy-undec-1-enyl)-3-methyl-phenyl]-propyl}-2-methyl-phenol (compound prepared in Example 178-(1)) (22 mg, 0.0474 mmol) in DMF (0.3 ml), K₂CO₃ (19.7 mg, 0.144 mmol) and (S)-(+)-dihydro-5-(p-toluenesulfonylmethyl)-2(3H)-furanone (25.6 mg, 0.0948 mmol) were added at room temperature and the mixture was stirred at 110 degrees C. for 16 h. To the mixture, ethyl acetate was added and the mixture was washed with H₂O, dried over MgSO₄, concentrated in vacuo. The obtained residue was chromatographed on silica gel (n-hexane:ethyl acetate=100:0 to 5:1) to give the title compound (9.9 mg, 37%).

¹H-NMR: 0.61 (t, 6H), 0.84-0.93 (m, 6H), 2.04 (q, 4H), 2.15 (s, 3H), 2.32 (s, 3H), 4.02-4.18 (m, 2H), 4.844.91 (m,1H), 6.01 (d,1H), 6.63-6.75 (m ,2H), 6.91-6.98 (m, 4H), 7.29 (d, 1H); MS (ESI+): 580 ([M+NH₄]⁺).

### Example 179

### Preparation of 5-(4-{1-Ethyl-1-[4-((E)-3-ethyl-3-hydroxy-hex-1-enyl)-3-methyl-phenyl]-propyl}-2-methyl-phenoxy)4(S)-hydroxy-pentanoic acid

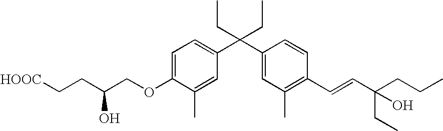

### (1) Preparation of 4-{1-Ethyl-1-[4-(3-ethyl-3-hydroxy-hex-1-enyl)-3-methyl-phenyl]-propyl}-2-methyl-phenol

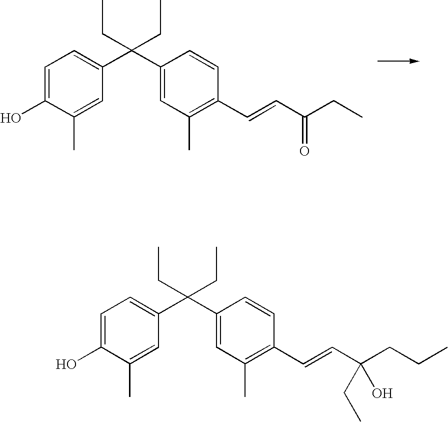

To a solution of (E)-1-{4-[1-Ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 0.93M n-propyl magnesium bromide in THF (1.08 ml, 1.00 mmol) were added at 0 degrees C. and the mixture was stirred at 0 degrees C. for 30 min and at room temperature for 2 h. To the mixture, saturated NH₄Cl solution was added and the mixture was filtrated through celite, concentrated in vacuo. The obtained residue was chromatographed on silica gel (n-hexane:ethyl acetate=100:0 to 50:50) to give the title compound (37 mg, 47%).

¹H-NMR: 0.57 (t, 6H), 0.83-0.92 (m, 6H), 2.00 (q, 4H), 4.98 (s, 1H), 6.01 (d, 1H), 6.61-7.30 (m, 7H).

### (2) Preparation of 5(S)-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one

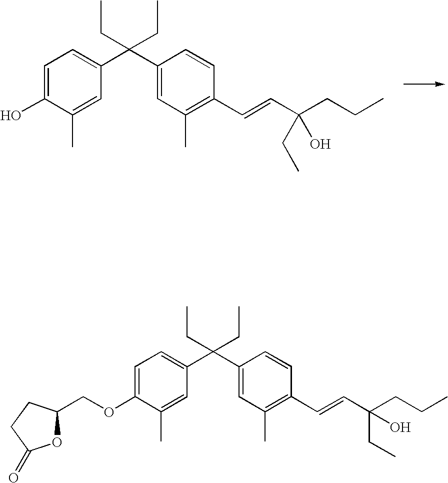

To a solution of 4-{I-Ethyl-1-[4-((E)-3-ethyl-3-hydroxy-undec-1-enyl)-3-methyl-phenyl]-propyl}-2-methyl-phenol compound prepared in Example 179-(1)) (18 mg, 0.0457 mmol) in DMF (0.4 ml), K₂CO₃ (16.6 mg, 0.120 mmol) and (S)-(+)-dihydro-5-(p-toluenesulfonylmethyl)-2(3H)-furanone (24.3 mg, 0.090 mmol) were added at room temperature and the mixture was stirred at 100 degrees C. for 60 h. To the mixture, ethyl acetate was added and the mixture was washed with H₂O, dried over MgSO₄, filtered and concentrated in vacuo. The obtained residue was chromatographed on silica gel (n-hexane:ethylacetate=l00:0 to 5:1) to give the title compound (13 mg, 58%).

¹H-NMR: 0.60 (t, 6H), 0.89-0.95 (m, 6H), 2.04 (q, 4H), 2.16 (s, 3H), 2.30 (s, 3H), 4.01-4.18 (m, 2H), 4.83-4.90 (m, 1H), 6.00 (d, 1H), 6.63-6.75 (m, 2H), 6.89-6.97 (m, 4H), 7.29 (d,1H); MS (ESI+) 475 ([M−OH]⁺).

### (3) Preparation of 5-(4-{1-Ethyl-1-[4-((E)-3-ethyl-3-hydroxy-hex-1-enyl)-3-methyl-phenyl]-propyl}-2-methyl-phenoxy)-4(S)-hydroxy-pentanoic acid

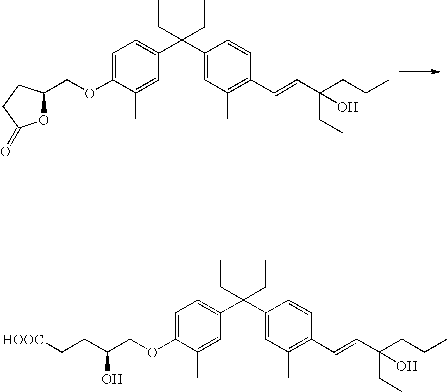

To a solution of 5(S)-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one (compound prepared in Example 179-(2.)) (13 mg, 0.026 mmol) in MeOH (1 ml), 1N NaOH (0.1 ml) was added at room temperature and the mixture was stirred at room temperature for 1 h. The mixture was concentrated in vacuo and purified by preparative TLC (CHCl₃:MeOH=8:3 saturated with H₂O) to give the title compound (2.0 mg, 15%).

¹H-NMR: 0.60 (t, 6H), 0.87-0.92 (m, 6H), 2.01 (q, 4H), 2.18 (s, 3H), 2.30 (s, 3H), 2.59 (t, 2H), 3.80-4.07 (m, 3H), 6.01 (d, I H), 6.64-6.75 (m, 2H), 6.88-6.97 (m, 4H), 7.25 (d, 1H); MS (ESI+): 528 ([M+NH₄]⁺).

### Example 180

### Preparation of 5-(4-{1-Ethyl-1-[4-((E)-3-ethyl-3-hydroxy-hept-1-enyl)-3-methyl-phenyl]-propyl}-2-methyl-phenoxy)4(S)-hydroxy-pentanoic acid

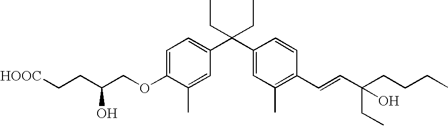

### (1) Preparation of 4-{1-Ethyl-1-[4-((E)-3-ethyl-3-hydroxy-hept-1-enyl)-3-methyl-phenyl]-propyl}-2-methyl-phenol

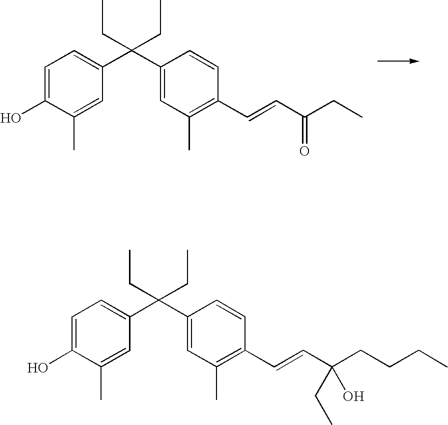

To a solution of (E)-1-{4-[1-Ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 1.58M n-butyl lithium in n-hexane (0.632 ml, 1.00 mmol) were added at 0 degrees C. and the mixture was stirred at 0 degrees C. for 30 min and at room temperature for 2 h. To the mixture, saturated NH₄Cl solution was added and the mixture was filtrated through celite, concentrated in vacuo. The obtained residue was chromatographed on silica gel (n-hexane:ethyl acetate=100:0 to 50:50) to give the title compound (51 mg, 62%).

¹H-NMR: 0.61 (t, 6H), 0.82-0.92 (m, 6H), 2.01 (q, 4H), 2.18 (s, 3H), 2.30 (s, 3H), 4.95 (s, 1H), 6.00 (d, 1H), 6.62-7.30 (m, 7H); MS (ESI+): 409 ([M+H]⁺).

### (2) Preparation of 5(S)-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one

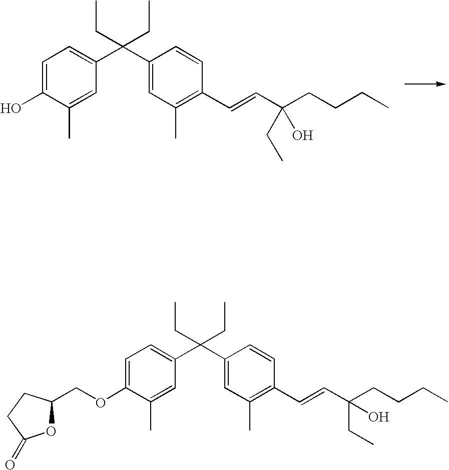

To a solution of 4-{I-Ethyl-1-[4-((E)-3-ethyl-3-hydroxy-hept-1-enyl)-3-methyl-phenyl]-propyl}-2-methyl-phenol (compound prepared in Example 180-(1)) (18 mg, 0.0441 mmol) in DMF (0.4 ml), K₂CO₃ (16.6 mg, 0.120 mmol) and (S)-(+)-dihydro-5-(p-toluenesulfonylmethyl)-2(3H)-furanone (24.3 mg, 0.090 mmol) were added at room temperature and the mixture was stirred at 100 degrees C. for 60 h. To the mixture, ethyl acetate was added and the mixture was washed with H₂O, dried over MgSO₄, filtered and concentrated in vacuo. The obtained residue was chromatographed on silica gel (n-hexane:ethylacetate=100:0 to 5:1) to give the title compound (20 mg, 90%).

¹H-NMR: 0.60 (t, 6H), 0.89-0.92 (m, 6H), 2.02 (q, 4H), 2.17 (s, 3H), 2.28 (s, 3H), 4.02-4.18 (m, 2H), 4.82-4.90 (m, 1H), 6.01 (d, 1H), 6.64-6.75 (m, 2H), 6.89-6.95 (m, 4H), 7.25 (d,1H); MS (ESI+): 489 ([M−OH]⁺).

### (3) Preparation of 5-(4-{1-Ethyl-1-[4-((E)-3-ethyl-3-hydroxy-hept-1-enyl)-3-methyl-phenyl]-propyl}-2-methyl-phenoxy)-4(S)-hydroxy-pentanoic acid

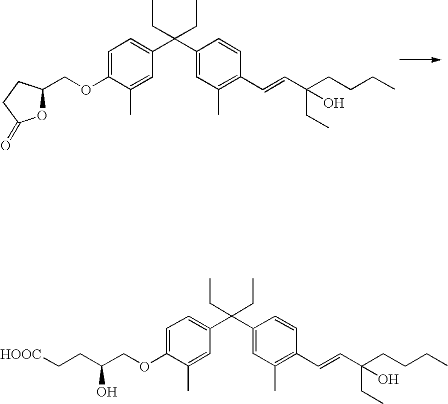

To a solution of 5(S)-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one (compound prepared in Example 180-(2)) (20 mg, 0.040 mmol) in MeOH (1 ml), 1 N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (7.1 mg, 34%).

¹H-NMR: 0.61 (t, 6H), 0.87-0.92 (m, 6H), 2.01 (q, 4H), 2.17 (s, 3H), 2.30 (s, 3H), 2.61 (t, 2H), 3.80-4.10 (m, 3H), 6.01 (d,1H), 6.63-6.75 (m, 2H), 6.83-6.96 (m, 4H), 7.26 (d,1H); MS (ESI+): 542 ([M+NH₄]⁺).

### Example 181

### Preparation of 5-(4-{1-Ethyl-1-[4-((E)-3-ethyl-3-hydroxy-oct-1-enyl)-3-methyl-phenyl]-propyl)-2-methyl-phenoxy)-4(S)-hydroxy-pentanoic acid

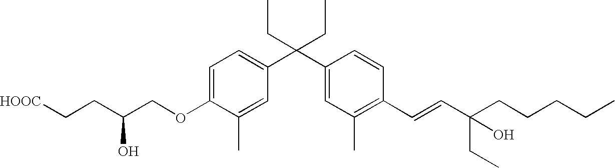

### (1) Preparation of 4-{1-Ethyl-1-[4-((E)-3-ethyl-3-hydroxy-oct-1-enyl)-3-methyl-phenyl]-propyl}-2-methyl-phenol

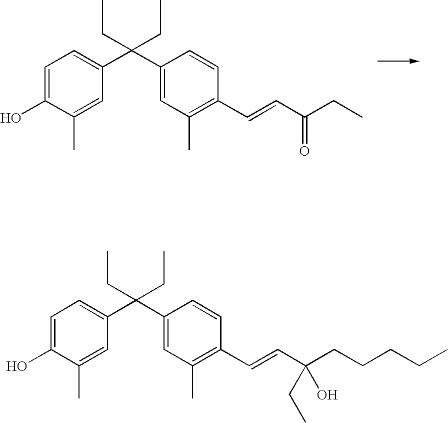

To a solution of (E)-1-{4-[1-Ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 2M pentyl magnesium bromide in THF (0.500 ml, 1.00 mmol) was added at 0 degrees C. and the mixture was stirred at 0 degrees C. for 30 min and at room temperature for 2 h. To the mixture, saturated NH₄Cl solution was added and the mixture was filtrated through celite, concentrated in vacuo. The obtained residue was chromatographed on silica gel (n-hexane:ethyl acetate=100:0 to 50:50) to give the title compound (23 mg, 27%).

¹H-NMR: 0.60 (t, 6H), 0.80-0.90 (m, 6H), 2.00 (q, 4H), 2.17 (s, 3H), 2.28 (s, 3H), 4.88 (s,1H), 6.00 (d,1H), 6.60-7.28 (m, 7H); MS (ESI+): 423 ([M+H]⁺).

### (2) Preparation of 5((S(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one

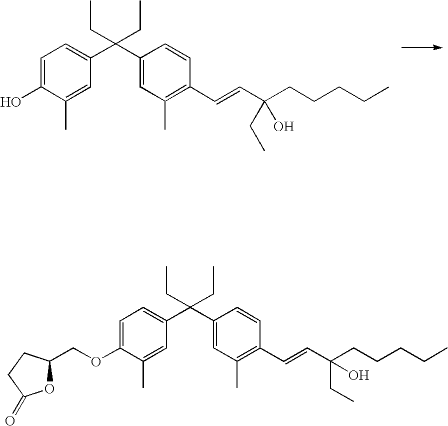

To a solution of 4-{1-Ethyl-1-[4-((E)-3-ethyl-3-hydroxy-oct-1-enyl)-3-methylphenyl]-propyl)-2-methyl-phenol (compound preprared in Example 181-(1)) (18 mg, 0.0427 mmol) in DMF (0.4 ml), K₂CO₃ (16.6 mg, 0.120 mmol) and (S)-(+)-dihydro-5-(p-toluenesulfonylmethyl)-2(3H)-furanone (24.3 mg, 0.090 mmol) were added at room temperature and the mixture was stirred at 1 00 degrees C. for 60 h. To the mixture, ethyl acetate was added and the mixture was washed with H₂O, dried over MgSO4, filtered and concentrated in vacuo. The obtained residue was chromatographed on silica gel (n-hexane:ethylacetate=100:0 to 5:1) to give the title compound (22 mg, 99%).

¹H-NMR: 0.61 (t, 6H), 0.83-0.92 (m, 6H), 2.03 (q, 4H), 2.12 (s, 3H), 2.30 (s, 3H), 4.01-4.18 (m,2H), 4.83-4.90 (m, 1H), 5.98 (d, 1H), 6.60-6.72 (m, 2H), 6.82-6.95 (m, 4H), 7.25 (d, 1H); MS (ESI+): 503 ([M−OH]⁺).

### (3) Preparation of 5-(4-{1-Ethyl-1-[4-((E)-3-ethyl-3-hydroxy-oct-1-enyl)-3-methyl-phenyl]-propyl}-2-methyl-phenoxy)4(S)-hydroxy-pentanoic acid

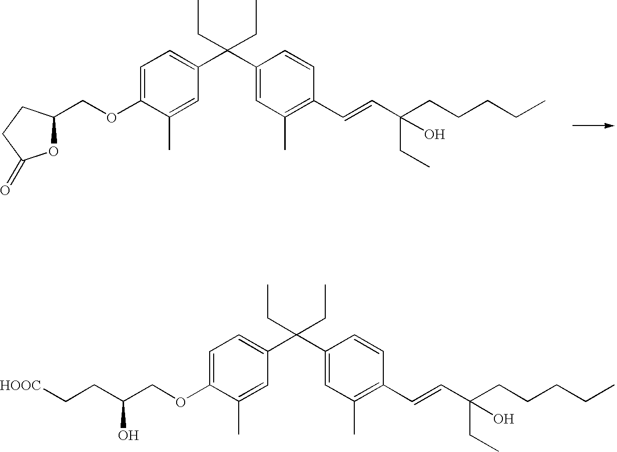

To a solution of (S)-5-(4-{l-Ethyl-1-[4-((E)-3-ethyl-3-hydroxy-oct-1-enyl)-3-methyl-phenyl]-propyl}-2-methyl-phenoxymethyl)-dihydro-furan-2-one (compound prepared in Example 181-(2)) (22 mg, 0.042 mmol) in MeOH (1 ml), 1 N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (1.8 mg, 8%).

¹H-NMR: 0.60 (t, 6H), 0.82-0.90 (m, 6H), 2.03 (q, 4H), 2.18 (s, 3H), 2.30 (s, 3H), 2.59 (t, 2H), 3.80-4.13 (m, 3H), 6.00 (d,1H), 6.65-6.75 (m, 2H), 6.90-7.00 (m, 4H), 7.26 (d,1H); MS (ESI+): 556 ([M+NH₄]⁺).

### Example 182

### Preparation of 5-(4-{1-Ethyl-1-[4-((E)-3-ethyl-3-hydroxy-non-1-enyl)-3-methyl-phenyl]-propyl}-2-methyl-phenoxy)-4(S)-hydroxy-pentanoic acid

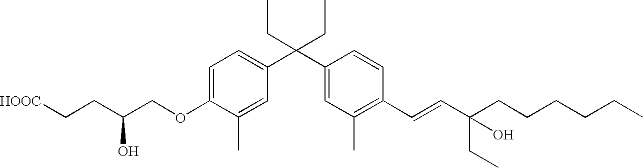

### (1) Preparation of 4-{1-Ethyl-1-[4-((E)-3-ethyl-3-hydroxy-non-1-enyl)-3-methyl-phenyl]-propyl}-2-methyl-phenol

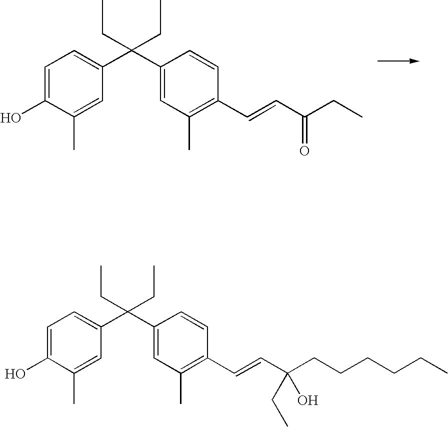

To a solution of (E)-1-{4-[1-Ethyl-1-(4-hydroxy-3-methyl-phenyl)-propyl]-2-methyl-phenyl}-pent-1-en-3-one (compound prepared in Example 177-(1)) (70 mg, 0.200 mmol) in THF (2 ml), 2M hexyl magnesium bromide in THF (0.500 ml, 1.00 mmol) were added at 0 degrees C. and the mixture was stirred at 0 degrees C. for 30 min and at room temperature for 2 h. To the mixture, saturated NH₄Cl solution was added and the mixture was filtrated through celite, concentrated in vacuo. The obtained residue was chromatographed on silica gel (n-hexane:ethyl acetate=100:0 to 50:50) to give the title compound (15 mg, 17%).

¹H-NMR: 0.62 (t, 6H), 0.82-0.90 (m, 6H), 2.00 (q, 4H), 2.17 (s, 3H), 2.28 (s, 3H), 4.70 (s,1H), 6.00 (d,1H), 6.60-7.28 (m, 7H); MS (ESI+): 437 ([M+H]⁺).

### (2) Preparation of 5(S)-(4-{1-Ethyl-1-[4-((E)-3-ethyl-3-hydroxy-non-1-enyl)-3-methyl-phenyl]-propyl}-2-methyl-phenoxymethyl)-dihydro-furan-2-one

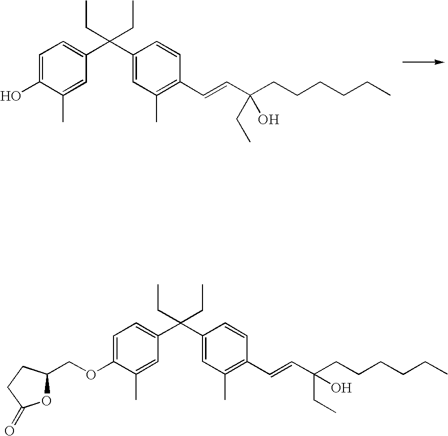

To a solution of 4-{1-Ethyl-1-[4-((E)-3-ethyl-3-hydroxy-non-1-enyl)-3-methyl-phenyl]-propyl}-2-methyl-phenol (compound prepared in Example 182-(1)) (18 mg, 0.0413 mmol) in OMF (0.4 ml), K₂CO₃ (16.6 mg, 0.120 mmol) and (S)-(+)-dihydro-5-(p-toluenesulfonylmethyl)-2(3H)-furanone (24.3 mg, 0.090 mmol) were added at room temperature and the mixture was stirred at 100 degrees C. for 60 h. To the mixture, ethyl acetate was added and the mixture was washed with H₂O, dried over MgSO₄, filtered and concentrated in vacuo. The obtained residue was chromatographed on silica gel (n-hexane:ethylacetate=100:0 to 5:1) to give the title compound (17 mg, 77%).

¹H-NMR: 0.62 (t, 6H), 0.82-0.92 (m, 6H), 2.05 (q, 4H), 2.15 (s, 3H), 2.30 (s, 3H), 4.01-4.18 (m, 2H), 4.82-4.90 (m,1H), 6.00 (d,1H), 6.61-6.72 (m, 2H), 6.88-6.95 (m, 4H), 7.26 (d,1H); MS (ESI+): 517 ([M−OH]⁺).

### (3) Preparation of 5-(4-{1-Ethyl-1-[4-((E)-3-ethyl-3-hydroxy-non-1-enyl)-3-methyl-phenyl]-propyl}-2-methyl-phenoxy)-4(S)-hydroxy-pentanoic acid

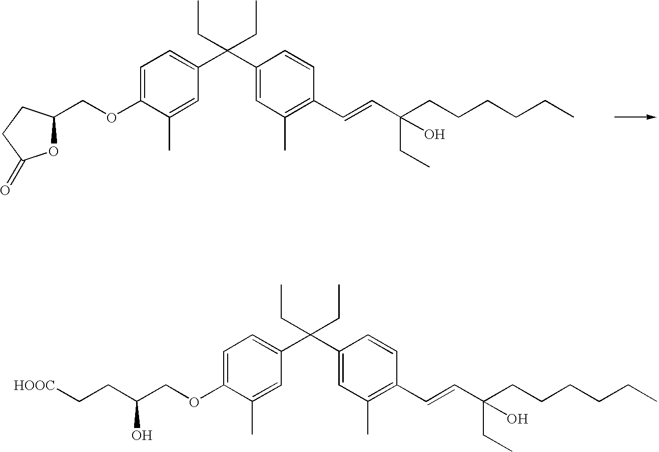

To a solution of 5(S)-(4-{1-Ethyl-1-[4-((E)-3-ethyl-3-hydroxy-non-1-enyl)-3-methyl-phenyl]-propyl}-2-methyl-phenoxymethyl )-dihydro-furan-2-one (compound prepared in Example 182-(2)) (17 mg, 0.032 mmol) in MeOH (1 ml), 1 N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (6.1 mg, 35%).

¹H-NMR: 0.62 (t, 6H), 0.84-0.94 (m, 6H), 2.03 (q, 4H), 2.18 (s, 3H), 2.30 (s, 3H), 2.61 (t, 2H), 3.80-4.10 (m, 3H), 6.01 (d,1H), 6.65-6.77 (m, 2H), 6.90-6.97 (m, 4H), 7.26 (d, 1H); MS (ESI+): 570 ([M+NH₄]⁺).

### Example 183

### Preparation of 5-(4-{1-Ethyl-1-[4-((E)-3-ethyl-3-hydroxy-dec-1-enyl)-3-methyl-phenyl]-propyl}-2-methyl-phenoxy)-4(S)-hydroxy-pentanoic acid

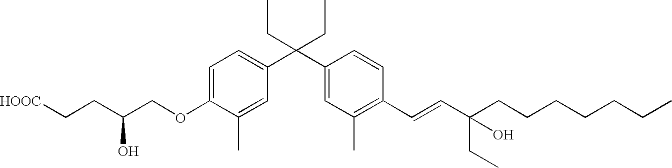

### Preparation of 5-(4-{1-Ethyl-1-[4-((E)-3-ethyl-3-hydroxy-dec-1-enyl)-3-methyl-phenyl]-propyl}-2-methyl-phenoxy)-4(S)-hydroxy-pentanoic acid

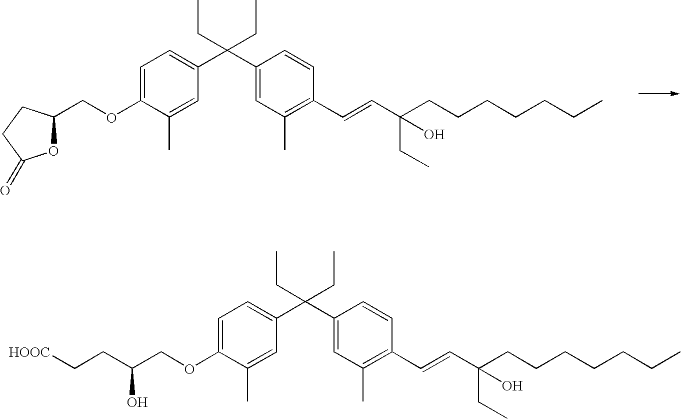

To a solution of compound prepared in Example 177-(3) ((S)-5-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one) (6.3 mg, 0.011 mmol) in MeOH (1 ml), 1N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (4.9 mg, 79%).

¹H-NMR: 0.60 (t, 6H), 0.86-0.92 (m, 6H), 2.01, (q, 4H), 2.12 (s, 3H), 2.30 (s, 3H), 2.57 (t, 2H), 3.80-4.08 (m, 3H), 6.00 (d, 1 H), 6.63-6.78 (m, 2H), 6.88-6.93 (m, 4H); MS (ESI+): 584 ([M+NH₄]⁺).

### Example 184

### Preparation of 5-(4-{1-Ethyl-1-[4-((E)-3-ethyl-3-hydroxy-undec-1-enyl)-3-methyl-phenyl]-propyl}-2-methyl-phenoxy)-4(S)-hydroxy-pentanoic acid

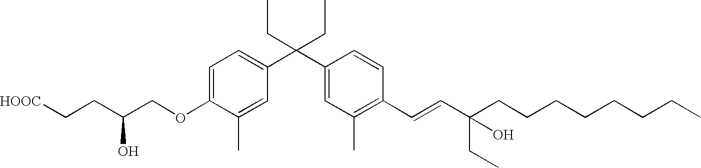

### Preparation of 5-(4-{1-Ethyl-1-[4-((E)-3-ethyl-3-hydroxy-undec-1-enyl)-3-methyl-phenyl]-propyl}-2-methyl-phenoxy)-4(S)-hydroxy-pentanoic acid

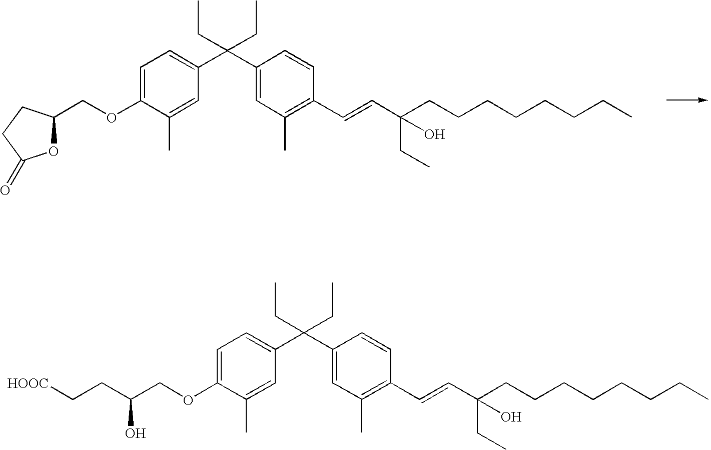

To a solution of 5(S)-(4-{1-Ethyl-1-[4-((E)-3-ethyl-3-hydroxy-undec-1-enyl)-3-methyl-phenyl]-propyl}-2-methyl-phenoxy)-4-hydroxy-pentanoic acid (compound prepared in Example 178-(2)) (7.0 mg, 0.012 mmol) in MeOH (1 ml), 1N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (3.7 mg, 55%).

¹H-NMR: 0.60 (t, 6H), 0.84-0.92 (m, 6H), 2.01 (q, 4H), 2.15 (s, 3H), 2.30 (s, 3H), 2.58 (t, 2H), 3.80-4.07 (m, 3H), 6.00 (d, 1H), 6.62-6.72 (m, 2H), 6.85-6.93 (m, 4H); MS (ESI+): 598 ([M+NH₄]⁺).

### Example 185

### Preparation of 5-(4-{1-Ethyl-1-[4-((E)-3-ethyl-3-hydroxy-oct-1-en-4-ynyl)-3-methyl-phenyl]-propyl}-2-methyl-phenoxy)-4(S)-hydroxy-pentanoic acid

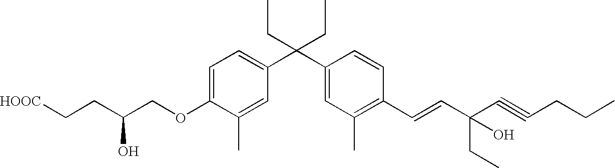

### (1) Preparation of 4-{1-Ethyl-1-[4-((E)-3-ethyl-3-hydroxy-oct-1-en-4-ynyl)-3-methyl-phenyl]-propyl}-2-methyl-phenol

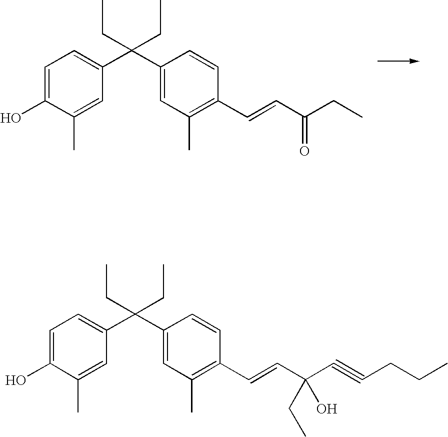

To a solution of 1-pentyne (0.0986 ml, 1.00 mmol) in THF (1 ml), 1.56M n-butyl lithium in n-hexane (0.646 ml, 1.02 mmol) was added at 0 degrees C. and the mixture was stirred at room temperature for 30 min. To the mixture, a solution of 4-{1-Ethyl-1-[4-((E)-3-ethyl-3-hydroxy-oct-1-en-4-ynyl)-3-methyl-phenyl]-propyl}-2-methyl-phenol (compound prepared in Example 177-(1)) (70 mg, 0.20 mmol) in THF (1 ml) was added at room temperature and the mixture was stirred at room temperature for 1 h. To the mixture, saturated NH₄Cl solution was added and the mixture was filtrated through celite, concentrated in vacuo. The obtained residue-was chromatographed on silica gel (n-hexane:ethyl acetate=100:0 to 50:50) to give the title compound (70 mg, 84%).

¹H-NMR: 0.59 (t, 6H), 2.00 (q, 4H), 2.19 (s, 3H), 2.24 (t, 2H), 2.30 (s, 3H), 4.67 (s, 1 H), 6.05 (d, 1 H), 6.63 (d, 1 H), 6.82-6.95 (m, 4H), 7.08 (d, 1 H), 7.36 (d, 1H); MS (ESI+): 419 ([M+H]⁺).

### (2) Preparation of 5(S)-(4-{1-Ethyl-1-[4-((E)-3-ethyl-3-hydroxy-oct-1-en-4-ynyl)-3-methyl-phenyl]-propyl}-2-methyl-phenoxymethyl)-dihydro-furan-2-one

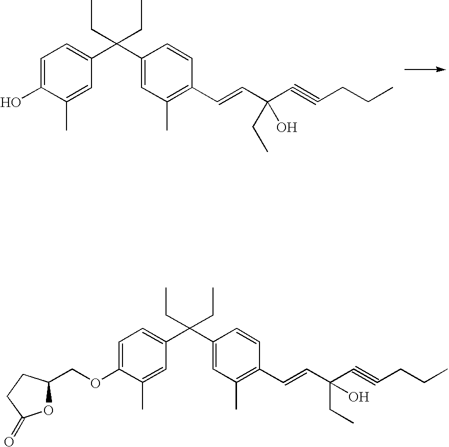

To a solution of 4-{1-Ethyl-1-[4-((E)-3-ethyl-3-hydroxy-oct-1-en-4-ynyl)-3-methyl-phenyl]-propyl}-2-methyl-phenol (compound prepared in Example 185-(1)) (15 mg, 0.0359 mmol) in DMF (0.4 ml), K₂CO₃ (16.6 mg, 0.120 mmol) and (S)-(+)-dihydro-5-(p-toluenesulfonylmethyl)-2(3H)-furanone (24.3 mg, 0.090 mmol) were added at room temperature and the mixture was stirred at 100 degrees C. for 60 h. To the mixture, ethyl acetate was added and the mixture was washed with H₂O, dried over MgSO₄, filtered and concentrated in vacuo. The obtained residue was chromatographed on silica gel (n-hexane:ethyl acetate=100:0 to 5:1) to give the title compound (18 mg, 97%).

¹H-NMR: 0.59 (t, 6H), 0.97-1.04 (m, 6H), 2.03 (q, 4H), 2.16 (s, 3H), 2.27 (t, 2H), 2.32 (s, 3H), 4.01-4.18 (m, 2H), 4.82-4.90 (m, 1H), 6.03 (d, 1H), 6.62 (d, 1H), 6.90-6.97 (m, 4H), 7.03 (d, 1 H), 7.37 (d, 1 H) MS (ESI+): 499 ([M−OH]⁺)

### (3) Preparation of 5-(4-{1-Ethyl-1-[4-((E)-3-ethyl-3-hydroxy-oct-1-en-4-ynyl)-3-methyl-phenyl]-propyl)-2-methyl-phenoxy)-4(S)-hydroxy-pentanoic acid

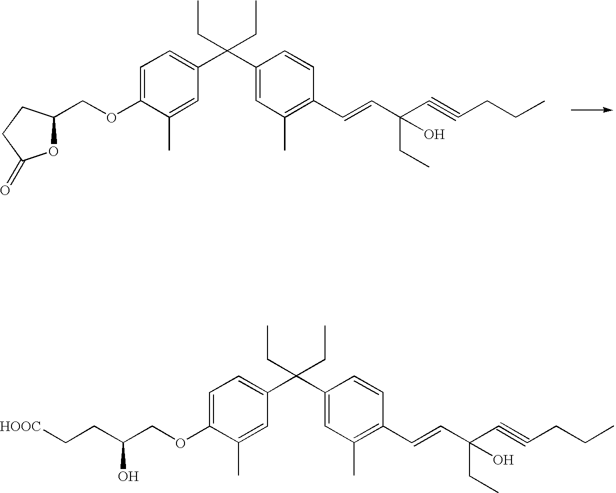

To a solution of 5(S)-(4-{1-Ethyl-1-[4-((E)-3-ethyl-3-hydroxy-oct-1-en-4-ynyl)-3-methyl-phenyl]-propyl}-2-methyl-phenoxymethyl )-dihydro-furan-2-one (compound prepared in Example 185-(2)) (18 mg, 0.035 mmol) in MeOH (1 ml), 1N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (5.2 mg, 29%).

¹H-NMR: 0.60 (t, 6H), 0.97-1.03 (m, 6H), 1.52-1.60 (m, 4H), 2.02 (q, 4H), 2.17 (s, 3H), 2.28 (t, 2H), 2.30 (s, 3H), 2.62 (t, 2H), 3.82-4.08 (m, 3H), 6.08 (d, 1H), 6.68 (d, 1H), 6.88-6.92 (m, 4H), 7.08 (d, 1H), 7.32 (d, 1H); MS (ESI+): 552 ([M+NH₄]⁺).

### Example 186

### Preparation of 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid

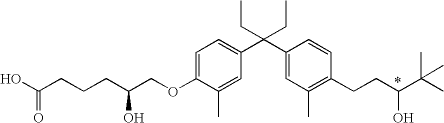

### (1) Preparation of 4-(1-{4-[3-(tert-Butyl-dimethyl-silanyloxy)-4,4-dimethyl-pentyl]-3-methyl-phenyl}-1-ethyl-propyl)-2-methyl-phenol

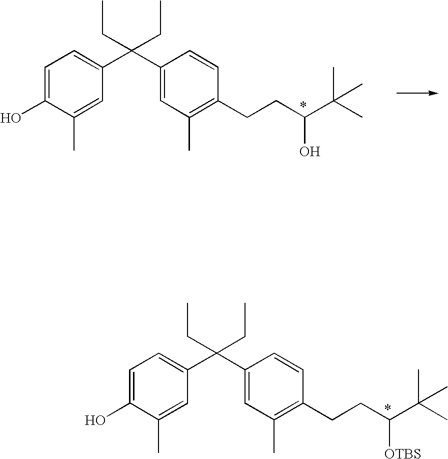

To a solution of 4-{1-Ethyl-1-[4-(3-hydroxy-4,4-dimethyl-pentyl)-3-methyl-phenyl]-propyl}-2-methyl-phenol (compound prepared in Example 20) (5.04 g, 13.2 mmol) in CH₂Cl₂ (40 ml) and lutidine (4.76 ml, 40.9 mmol), Trifluoromethanesulfonic acid tert-butyldimethylsilyl ester (0.51 ml, 3.09 mmol) was added at 0 degrees C. and the mixture was stirred for 0.5 h at 0 degrees C. The reaction mixture was poured into H₂O and the products were extracted with ethyl acetate and hexane. The extracts were washed with saturated aqueous NH₄Cl, dried over MgSO₄, filtered and concentrated in vacuo. The obtained residue was chromatographed on silica gel (ethyl acetate/hexane=0/100 to 10/90) to give the disilylated compound (7.32 g). This disilylated compound was dissolved with THF (113 ml) and 1 M TBAF in THF solution (12.6 ml, 12.6 mmol) was added at 0 degrees C. and the mixture was stirred for 5 min at 0 degrees C. The reaction mixture was poured into brine and the products were extracted with ethyl acetate and hexane. The extracts were washed with brine twice, dried over MgSO₄, filtered and concentrated in vacuo. The obtained residue was chromatographed on silica gel (ethyl acetate/hexane=10/90 to 20/80) to give the title compound (5.66 g, 86%).

¹H-NMR: 0.07 (s, 3H), 0.10 (s, 3H), 0.60 (t, 6H), 0.88 (s, 9H), 0.93 (s, 9H), 1.51-1.63 (m, 1 H), 1.73-1.84 (m, 1 H), 2.03 (q, 4H), 2.20 (s, 3H), 2.23 (s, 3H), 2.36-2.46 (m, 1H), 2.71-2.81 (m, 1H), 3.34 (dd, 1H), 4.50 (s, 1H), 6.64 (d, 1H), 6.85-6.99 (m, 5H).

### (2) Preparation of 6(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 186-(1)) (55.7 mg, 0.112 mmol), PPh₃ (88.2 mg, 0.336 mmol), (S)-6-Hydroxymethyl-tetrahydro-pyran-2-one (J. Chem. Soc., Perkin Trans. 1, 2000, 20, 3519.) (43.7 mg, 0.336 mmol) in THF (0.2 ml), DEAD (58.6 mg, 0.336 mmol) was added slowly and the mixture was stirred at room temperature for 15 h. The mixture was concentrated in vacuo and purified by preparative TLC (n-hexane/ethyl acetate=1/1 and 3/1) to give the title compound (5.9 mg, 8.6%).

¹H-NMR (chloroform-d): 0.07 (s, 3H), 0.11 (s, 3H), 0.60 (t, 6H, J=7.3 Hz), 0.88 (s, 9H), 0.94 (s, 9H), 1.72-2.13 (m, 6H), 2.04 (q, 4H, J=7.3 Hz), 2.16 (s, 3H), 2.24 (s, 3H), 2.36-2.81 (m, 4H), 3.34 (dd, 1H, J=3.2 Hz), 4.04 (dd, 1H, J=5.6, 9.8 Hz), 4.13 (dd, 1H, J=4.3, 9.8 Hz), 4.64-4.71 (m, 1H), 6.67 (d, 1H, J=8.3 Hz), 6.89-7.00 (m, 5H).

### (3) Preparation of 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid

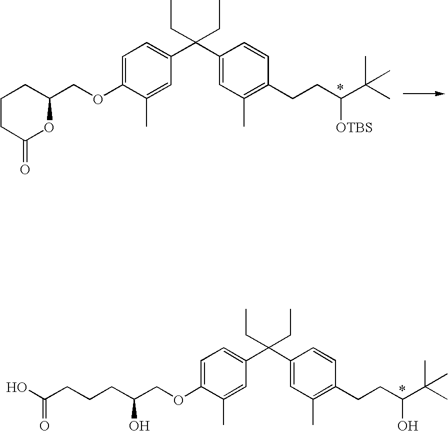

To a solution of 6(S)-[4-(1-{4-[3-(tert-Butyl-dimethyl-silanyloxy)-4,4-dimethyl-pentyl]-3-methyl-phenyl-1-ethyl-propyl)-2-methyl-phenoxymethyl]-tetrahydro-pyran-2-one (compound prepared in Example 186-(2)) (5.9 mg, 0.01 mmol) in THF (0.1 ml), 1.0M TBAF (0.24 ml, 0.24 mmol) was added and the mixture was stirred at 70 degrees C. for 7 h. To the mixture, ethyl acetate was added and the mixture was washed with diluted potassium hydrogen sulfate aq., saturated sodium hydrogen carbonate solution, and brine, dried over magnesium sulfate, filtered and concentrated in vacuo. The obtained residue was chromatographed on silica gel (dichrolomethane/methanol=15/1) to give the title compound (2.6 mg, 52.3%).

¹H-NMR (methanol-d₄): 0.58 (t, 6H, J=7.3 Hz), 0.82 (s, 9H), 1.45-1.84 (m, 6H), 0.00 (q, 4H, J=7.3 Hz), 2.14 (s, 3H), 2.23 (s, 3H), 2.34 (t, 2H, J=7.2 Hz), 2.48-2.58 (m, 1H), 2.81-2.91 (m, 1H), 3.15 (dd, 1H, J=1.5, 10.5 Hz), 3.87-3.92 (m, 3H), 6.74 (d, 1H, J=8.5 Hz), 6.84-6.96 (m, 4H), 7.00 (d, 1H, J=7.9 Hz); MS (ESI+): 535 ([M+Na]⁺).

### Example 187

### Preparation of 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid

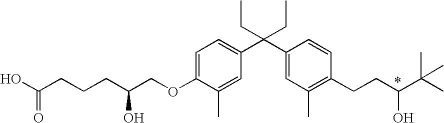

### (1) Preparation of 4-(1-{4-[3-(tert-Butyl-dimethyl-silanyloxy)-4,4-dimethyl-pentyl]-3-methyl-phenyl}-1-ethyl-propyl)-2-methyl-phenol

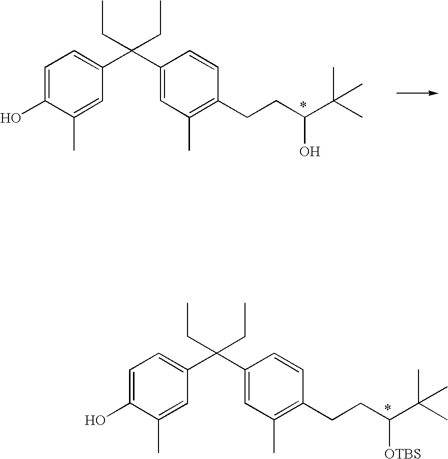

Using the same procedure as described for the preparation of Example 186-(1), the title compound was prepared from 4-{1-Ethyl-1-[4-(3-hydroxy-4,4-dimethyl-pentyl)-3-methyl-phenyl]-propyl}-2-methyl-phenol (compound prepared in Example 19).

¹H-NMR: 0.07 (s, 3H), 0.10 (s, 3H), 0.60 (t, 6H), 0.88 (s, 9H), 0.93 (s, 9H); 1.51-1.63 (m, 1H), 1.73-1.84 (m, 1H), 2.03 (q, 4H), 2.20 (s, 3H), 2.23 (s, 3H), 2.36-2.46 (m, 1H), 2.71-2.81 (m, 1H), 3.34 (dd, 1H), 4.50 (s, 1H), 6.64 (d, 1H), 6.85-6.99 (m, 5H).

### (2) Preparation of 6(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one

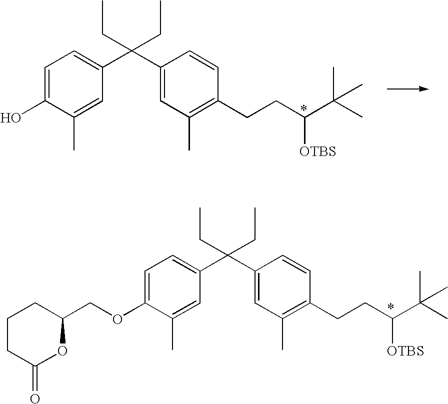

To a solution of 4-(1-{4-[(S)-3-(tert-Butyl-dimethyl-silanyloxy)-4,4-dimethyl-pentyl]-3-methyl-phenyl}-1-ethyl-propyl)-2-methyl-phenol (compound prepared in Example 187-(1)) (45.1 mg, 0.091 mmol), PPh₃ (71.4 mg, 0.272 mmol) and (S)-6-Hydroxymethyl-tetrahydro-pyran-2-one (J. Chem. Soc., Perkin Trans. 1, 2000, 20, 3519.) (35.4 mg, 0.272 mmol) in THF (0.15 ml), DEAD (47.4 mg, 0.272 mmol) was added slowly and the mixture was stirred at room temperature for 15 h. The mixture was concentrated in vacuo and purified by preparative TLC (n-hexane/ethyl acetate=3/1) to give the title compound (10 mg, 18.1%).

¹H-NMR (chloroform-d): 0.07 (s, 3H), 0.11 (s, 3H), 0.60 (t, 6H, J=7.3 Hz), 0.88 (s, 9H), 0.94 (s, 9H), 1.73-2.13 (m, 6H), 0.00 (q, 4H, J=7.3 Hz), 2.36-2.81 (m, 4H), 3.35 (dd, 1H, J=3.3, 7.1 Hz), 4.04 (dd, 1H, J=5.6, 9.8 Hz), 4.13 (dd, 1H, J=4.3, 9.8 Hz), 4.66-4.71 (m, 1H), 6.67 (d, 1H, J=8.2 Hz), 6.89-6.93 (m, 4H), 6.98 (d, 1H, J=8.5 Hz).

### (3) Preparation of 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid

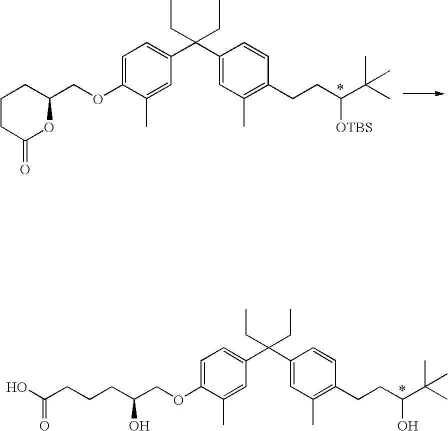

To a solution of 6(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one (compound prepared in Example 187-(2)) (10 mg, 0.016 mmol) in THF (0.2 ml), 1.0M TBAF in THF (0.4 ml, 0.4 mmol) was added and the mixture was stirred at 70 degrees C. for 4 h. To the mixture, ethyl acetate was added and the mixture was washed with diluted potassium hydrogen sulfate aq., saturated sodium hydrogen carbonate solution and brine, dried over magnesium sulfate, filtered and concentrated in vacuo. The obtained residue was purified by preparative TLC (dichrolomethane/methanol=15/1) to give the title compound (3.3 mg, 39.2%).

¹H-NMR (methanol-d₄): 0.58 (t, 6H, J=7.3 Hz), 0.86 (s, 9H), 1.41-1.84 (m, 6H), 2.05 (q, 4H, J=7.3 Hz), 2.14 (s, 3H), 2.28 (s, 3H), 2.35 (t, 2H, J=7.0 Hz), 6.74 (m, 1H), 2.81-2.91 (m, 1H), 3.15 (dd, 1H, J=1.6, 10.6 Hz), 3.87-3.93 (m, 3H), 6.74 (d, 1H, J=8.5 Hz), 6.84-6.96 (m, 4H), 7.00 (d, 1H, J=8.0 Hz); MS (ESI+): 535 ([M+Na]⁺).

### Example 188

### Preparation of 6-(4-{1-Ethyl-1-[4-(3-hydroxy-4,4-dimethyl-pentyl)-3-methyl-phenyl]-propyl}-2-methyl-phenoxy)-5(R)-hydroxy-hexanoic acid

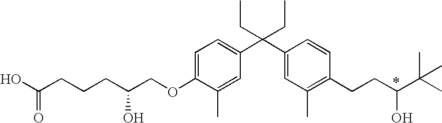

### (1) Preparation of 6(R)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one

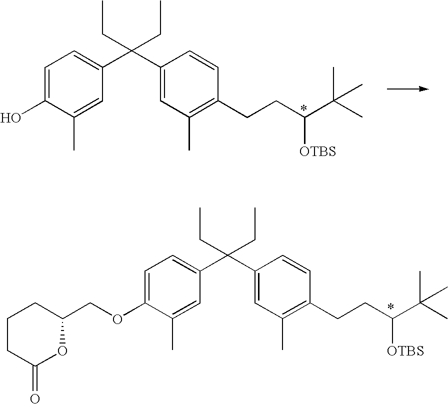

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 187-(1)) (40 mg, 0.081 mmol), PPh₃ (63.3 mg, 0.242 mmol) and (R)-6-Hydroxymethyl-tetrahydro-pyran-2-one (J. Chem. Soc., Perkin Trans. 1, 2000, 20, 3519.) (31.4 mg, 0.242 mmol) in THF (0.1 ml), DEAD (42.1 mg, 0.242 mmol) was added and the mixture was stirred at room temperature for 37 h. The mixture was concentrated in vacuo and purified by preparative TLC (n-hexane/ethyl acetate=3/1 and 2/1) to give the title compound (13.5 mg, 27.4%).

¹H-NMR (chloroform-d): 0.07 (s, 3H), 0.11 (s, 3H), 0.60 (t, 6H), 0.89 (s, 9H), 0.94 (s, 9H), 1.73-2.21 (m, 6H), 2.04 (q, 4H, J=7.5 Hz), 2.16 (s, 3H), 2.24 (s, 3H), 2.36-2.81 (m, 4H), 3.35 (dd, 1H, J=3.2, 7.1 Hz), 4.04 (dd, 1H, J=5.4, 9.9 Hz), 4.13 (dd, 1H, J=4.4, 9.8 Hz), 4.64-4.72 (m, 1H), 6.67 (d, 1H, J=8.1 Hz), 6.89-6.94 (m, 4H), 6.98 (d, 1H, J=8.7 Hz).

### (2) Preparation of 6-(4-{1-Ethyl-1-[4-(3-hydroxy-4,4-dimethyl-pentyl)-3-methyl-phenyl]-propyl}-2-methyl-phenoxy)-5(R)-hydroxy-hexanoic acid

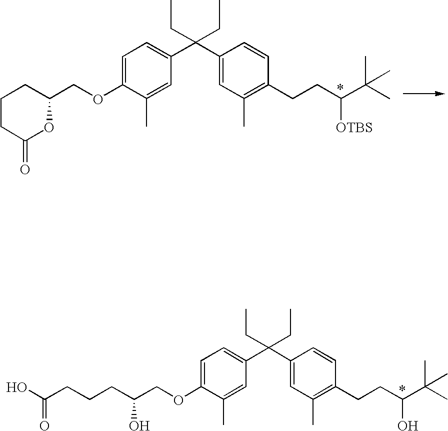

To a solution of 6(R)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one (compound prepared in Example 188-(1)) (16.5 mg, 0.033 mmol) in THF (0.2 ml) and MeOH (0.2 ml), 1N KOH aq (0.2 ml, 0.2 mmol) was added and the mixture was stirred at 65 degrees C. for 1.5 h. To the mixture, ethyl acetate was added and the mixture was washed with diluted potassium hydrogen sulfate aq., water, saturated sodium hydrogen carbonate solution and brine, dried over magnesium sulfate, filtered and concentrated in vacuo. The obtained residue was purified by preparative TLC (dichrolomethane/methanol=10/1) to give the title compound (8.4 mg, 49.1%).

¹H-NMR (methanol-d₄): 0.57 (t, 6H, J=7.3 Hz), 0.86 (s, 9H), 1.40-1.90 (m, 6H), 2.04 (q, 4H, J=7.3 Hz), 2.13 (s, 3H), 2.23 (s, 3H), 2.36 (brt, 2H), 2.47-2.58 (m, 1H), 2.81-2.91 (m, 1H), 3.15 (dd, 1H, J=1.5, 10.4 Hz), 3.86-3.98 (m, 3H), 6.73 (d, 1H, J=8.4 Hz), 6.84-6.96 (m, 4H), 7.00 (d, 1H, J=8.1 Hz); MS (ESI+): 530 ([M+NH₄]⁺).

### Example 189

### Preparation of 6-(4-{1-Ethyl-1-[4-(3-hydroxy-4,4-dimethyl-pentyl)-3-methyl-phenyl]-propyl}-2-methyl-phenoxy)-5(R)-hydroxy-hexanoic acid

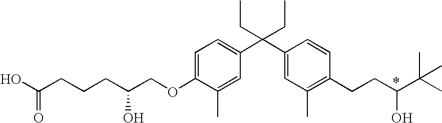

### (1) Preparation of 6(R)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one

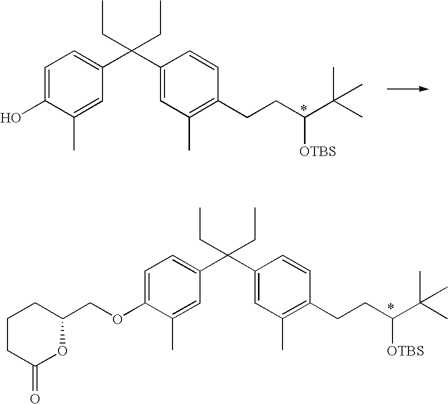

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 186-(1)) (40 mg, 0.081 mmol) PPh₃ (63 mg, 0.242 mmol), (R)-6-Hydroxymethyl-tetrahydro-pyran-2-one (J. Chem. Soc., Perkin Trans. 1, 2000, 20, 3519.) (31 mg, 0.242 mmol) in THF (0.1 ml), DEAD (42 mg, 0.242 mmol) was added and the mixture was stirred at room temperature for 37 h. The mixture was concentrated in vacuo and purified by preparative TLC (n-hexane/ethyl acetate=3/1 and 2/1) to give the title compound (13.5 mg, 27.5%).

¹H-NMR (chloroform-d): 0.07 (s, 3H), 0.10 (s, 3H), 0.60 (t, 6H), 0.88 (s, 9H), 0.94 (s, 9H), 1.73-2.16 (m, 6H), 2.04 (q, 4H, J=7.5 Hz), 2.16 (s, 3H), 2.24 (s, 3H), 2.36-2.81 (m, 4H), 3.35 (dd, 1H, J=3.3, 7.1 Hz), 4.04 (dd, 1H, J=5.6, 9.8 Hz), 4.13 (dd, 1H, J=4.3, 9.9 Hz), 4.64-4.72 (m, 1H), 6.67 (d, 1H, J=8.2 Hz), 6.89-6.94 (m, 4H), 6.98 (d, 1H, J=8.5 Hz).

### (2) Preparation of 6-(4-{1-Ethyl-1-[4-(3-hydroxy-4,4-dimethyl-pentyl)-3-methyl-phenyl]-propyl}-2-methyl-phenoxy)-5(R)-hydroxy-hexanoic acid

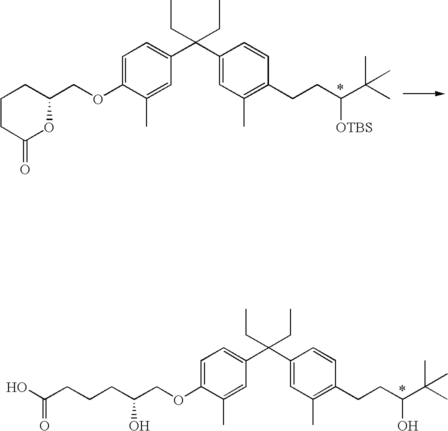

To a solution of 6(R)-[4-(1-{4-[(R)-3-(tert-Butyl-dimethyl-silanyloxy)-4,4-dimethyl-pentyl]-3-methyl-phenyl}-1-ethyl-propyl)-2-methyl-phenoxymethyl]-tetrahydro-pyran-2-one (compound prepared in Example 189-(1)) (16.5 mg, 0.027 mmol) in THF (0.2 ml) and MeOH (0.2 ml), 1N KOH aq (0.2 ml, 0.2 mmol) was added at room temperature and the mixture was stirred at 65 degrees C. for 1.5 h. To the mixture, ethyl acetate was added and the mixture was washed with diluted potassium hydrogen sulfate aq., water, saturated sodium hydrogen carbonate solution and brine, dried over magnesium sulfate, filtered and concentrated in vacuo. The obtained residue was purified by preparative TLC (dichrolomethane/methanol=10/1, twice) to give the title compound (6.9 mg, 49.7%).

¹H-NMR (methanol-d4): 0.58 (t, 6H, J=7.3 Hz), 0.86 (s, 9H), 1.38-1.84 (m, 6H), 2.05 (q, 4H, J=7.3 Hz), 2.14 (s, 3H), 2.23 (s, 3H), 2.34 (t, 2H, J=7.0 Hz), 2.47-2.58 (m, 1H), 2.81-2.91 (m, 1H), 3.15 (dd, 1H, J=1.5, 10.5 Hz), 3.87-3.98 (m, 3H), 6.73 (d, 1H, J=8.6 Hz), 6.84-6.96 (m, 4H), 7.00 (d, 1H, J=7.9 Hz); MS (ESI+): 513 ([M+H]⁺).

### Example 190

### Preparation of 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one

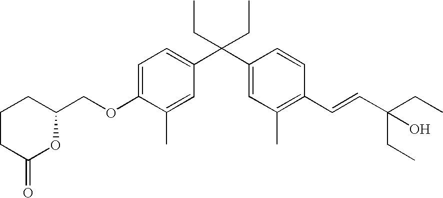

### Preparation of 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one

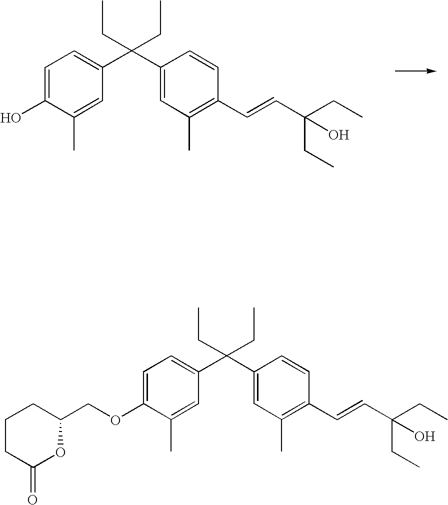

To a solution of 4-{1-Ethyl-1-[4-((E)-3-ethyl-3-hydroxy-pent-1-enyl)-3-methyl-phenyl]-propyl}-2-methyl-phenol (compound prepared in Example 1-(5)) (30 mg, 0.079 mmol), PPh₃ (62.0 mg, 0.236 mmol), (R)-6-Hydroxymethyl-tetrahydro-pyran-2-one (J. Chem. Soc., Perkin Trans. 1, 2000, 20, 3519.) (30.8 mg, 0.236 mmol) in THF (0.1 ml), DEAD (41.2 mg, 0.236 mmol) was added slowly and the mixture was stirred at room temperature for 37 h. The mixture was concentrated in vacuo and purified by preparative TLC (n-hexane/ethyl acetate=1/3, 3/1, 3 times, 1/1, dichloromethane/methanol=15/1) to give the title compound (8.5 mg, 21.9%).

¹H-NMR (chloroform-d): 0.61 (t, 6H, J=7.3 Hz), 0.92.(t, 6H, J=7.5 Hz), 1.64 (q, 4H, J=7.6 Hz), 1.82-1.95 (m, 4H), 2.05 (q, 4H, J=7.3 Hz), 2.16 (s, 3H), 2.31 (s, 3H), 2.46-2.71 (m, 2H), 4.04 (dd, 1H, J=5.5, 9.9 Hz), 4.13 (dd, 1H, J=4.3, 9.9 Hz), 4.65-4.72 (m, 1H), 6.01 (d, 1H, J=16.1 Hz), 6.67 (d, 1H, J=8.5 Hz), 6.74 (d, 1H, J=16.0 Hz), 6.92-6.97 (m, 4H), 7.29 (d, 1H, J=8.6 Hz); MS (ESI+) : 510 ([M+NH₄]⁺).

### Example 191

### Preparation of 6-(4-{1-Ethyl-1-[4-((E)-3-ethyl-3-hydroxy-pent-1-enyl)-3-methyl-phenyl]-propyl}-2-methyl-phenoxy)-5(R)-hydroxy-hexanoic acid

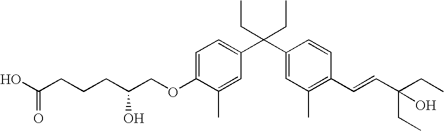

### Preparation of 6-(4-{1-Ethyl-1-[4-((E)-3-ethyl-3-hydroxy-pent-1-enyl)-3-methyl-phenyl]-propyl}-2-methyl-phenoxy)-5(R)-hydroxy-hexanoic acid

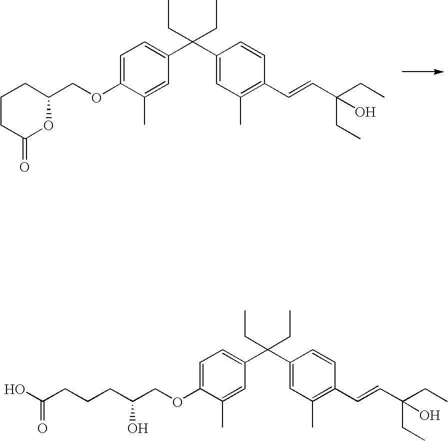

To a solution of 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one (compound prepared in Example 190) (5.9 mg, 0.012 mmol) in THF (0.1 ml) and MeOH (0.1 ml), 1N KOH aq (0.1 ml, 0.1 mmol) was added at room temperature and the mixture was stirred at 70 degrees C. for 4 h. To the mixture, ethyl acetate was added and the mixture was washed with diluted potassium hydrogen sulfate aq. and brine, dried over magnesium sulfate, filtered and concentrated in vacuo. The obtained residue was purified by preparative TLC (dichrolomethane/methanol=15/1, twice) to give the title compound (4.9 mg, 80.1%).

¹H-NMR (methanol-d₄): 0.59 (t, 6H, J=8.1 Hz), 0.91 (t, 6H, J=8.3 Hz), 1.62 (q, 4H, J=8.3 Hz), 1.52-1.82 (m, 4H), 2.06 (q, 4H, J=8.1 Hz), 2.14 (s, 3H), 2.21 (t, 2H, J=8.0 Hz), 2.27 (s, 3H), 3.82-3.98 (m, 3H), 5.98 (d, 1H, J=17.8 Hz), 6.75 (d, 1H, J=17.9 Hz), 6.75 (d, 1H, J=9.3 Hz), 6.91-6.96 (m, 3H), 7.27 (d, 1H, J=9.2 Hz); MS (ESI+): 493 ([M−OH]⁺).

### Example 192

### Preparation of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid

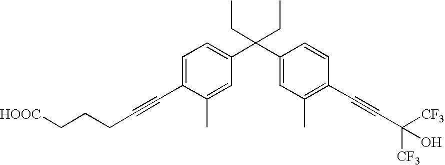

### (1) Preparation of Trifluoro-methanesulfonic acid 4-{1-ethyl-1-[3-methyl-4-(4,4,4-trifluoro-3-methoxymethoxy-3-trifluoromethyl-but-1-ynyl)-phenyl]-propyl)-2-methyl-phenyl ester

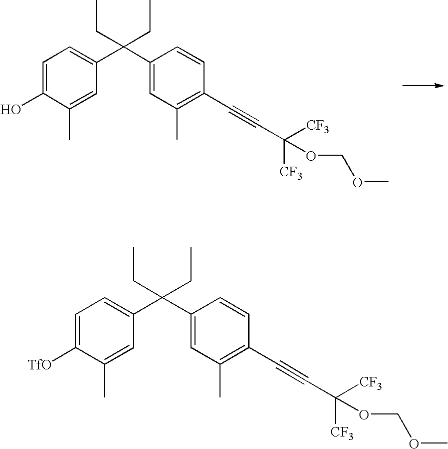

To a solution of 4-{i-Ethyl-i-[3-methyl-4-(4,4,4-trifluoro-3-methoxymethoxy-3-trifluoromethyl-but-1-ynyl)-phenyl]-propyl}-2-methyl-phenol (compound prepared in Example 2-(2)) (200 mg, 0.398 mmol) in CH₂Cl₂ (2 ml), pyridine (63.8 mg, 0.796 mmol) and trifluoromethanesulfonic anhydride (0.0737 ml, 0.438 mmol) were added and the mixture was stirred at room temperature for 16 h. The mixture was chromatographed on silica gel (ethyl acetate/n-hexane=0/100 to 1/5) to give the title compound (197 mg, 78%).

¹H-NMR: 0.60 (t, 6H), 2.06 (q, 4H), 2.32 (s, 3H), 2.41 (s, 3H), 3.48 (s, 3H), 5.15 (s, 2H), 6.94-7.05 (m, 4H), 7.12 (d, 1H), 7.30 (d, 1H).

### (2) Preparation of Hex-5-ynoic acid methyl ester

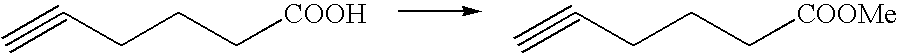

To a solution of hex-5-ynoic acid (2.0 g, 17.9 mmol) in MeOH (4 ml), conc. H₂SO₄ (4 drops) was added at room temperature and the mixture was stirred at 60 degrees C. for 60 h. To the mixture, ethyl acetate was added and the mixture was washed with saturated NaHCO₃, dried over MgSO₄ filtered and concentrated in vacuo to give the title compound (2.0 g, 89%).

¹H-NMR: 1.80-1.90 (m, 2H), 1.97-1.99 (m, 1H), 2.23-2.30 (m, 2H), 2.48 (t, 2H), 3.68 (s, 3H)

### (3) Preparation of 6-(4-{1-Ethyl-1-[3-methyl-4-(4,4,4-trifluoro-3-methoxymethoxy-3-trifluoromethyl-but-1-ynyl)-phenyl]-propyl}-2-methyl-phenyl)-hex-5-ynoic acid methyl ester

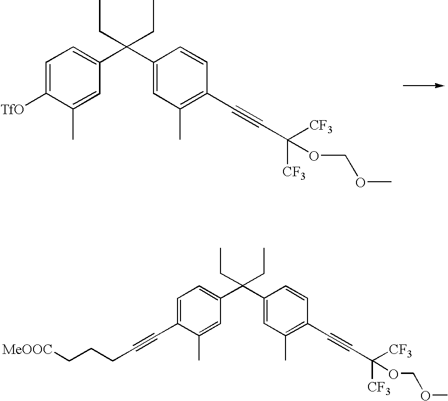

To a solution of trifluoro-methanesulfonic acid 4-{1-ethyl-1-[3-methyl-4-(4,4,4-trifluoro-3-methoxymethoxy-3-trifluoromethyl-but-1-ynyl)-phenyl]-propyl}-2-methyl-phenyl ester (compound prepared in Example 192-(1)) (23 mg, 0.0363 mmol) in DMF (0.5 ml), Et₃N (0.0253 ml, 0.182 mmol), Cul (3.4 mg, 0.0181 mmol), PdCl₂(PPh₃)₂ (12.7 mg, 0.0181 mmol) and hex-5-ynoic acid methyl ester (compound prepared in Example 192-(2)) (13.7 mg, 0.109 mmol) were added at room temperature and the mixture was stirred at 160 degrees C. for 30 min. To the mixture, ethyl acetate was added and the mixture was washed with H₂O, dried over MgSO₄, filtered and concentrated in vacuo. The obtained residue was purified by preparative TLC (n-hexane:ethyl acetate=5:1) to give the title compound (8.5 mg, 38%).

¹H-NMR: 0.59 (t, 6H), 1.91 (t, 2H), 2.08 (q, 4H), 2.34 (s, 3H), 2.39 (s, 3H), 2.53 (t, 2H), 3.48 (s, 2H), 3.68 (s, 3H), 5.15 (s, 2H), 6.87-7.38 (m, 6H).

### (4) Preparation of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid methyl ester

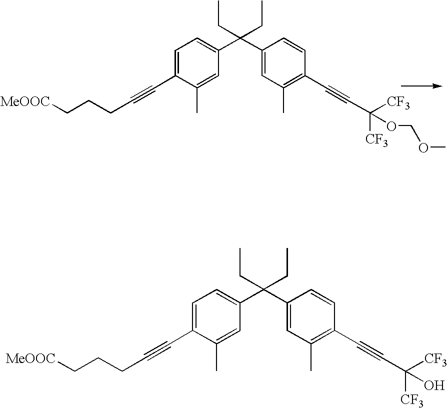

6-(4-{1-ethyl-1-[3-methyl-4-(4,4,4-trifluoro-3-methoxymethoxy-3-trifluoromethyl-but-1-ynyl)-phenyl]-propyl}-2-methyl-phenyl)-hex-5-ynoic acid methyl ester (compound prepared in Example 192-(3)) (8.5 mg, 0.0139 mmol) was dissolved in 4N HCl/dioxane (0.5 ml) at room temperature, and the mixture was stirred at room temperature for 16 h. The mixture was concentrated in vacuo and purified by preparative TLC (n-hexane:ethyl acetate=5:1) to give the title compound (6.3 mg, 80%).

¹H-NMR: 0.60 (t, 6H), 1.92 (t, 2H), 2.04 (q, 4H), 2.35 (s, 3H), 2.37 (s, 3H), 2.52 (t, 2H), 3.70 (s, 3H), 6.83-7.39 (m, 6H); MS (ESI+): 584 ([M+NH₄]⁺).

### (5) Preparation of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid

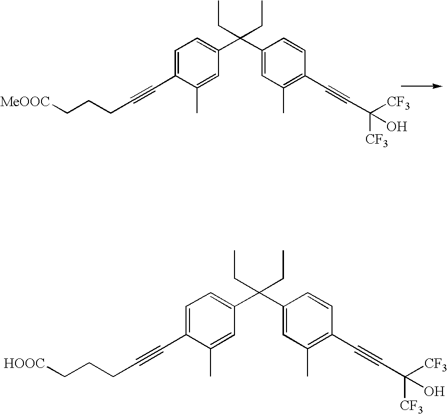

To a solution of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid methyl ester (compound prepared in Example 192-(4)) (6.3 mg, 0.0111 mmol) in MeOH (0.5 ml), 1N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (2.4 mg, 39%).

¹H-NMR: 0.61 (t, 6H), 1.90-1.99 (m, 2H), 2.06 (q, 4H), 2.35 (s, 3H), 2.37 (s, 3H), 2.52-2.60 (m, 2H), 6.85-7.02 (m, 4H), 7.35 (d, 1H); MS (ESI+): 570 ([M+NH₄]⁺).

### Example 193

### Preparation of 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid

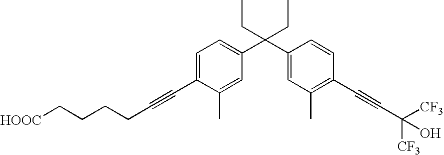

### (1) Preparation of-Hept-6-ynoic acid methyl ester

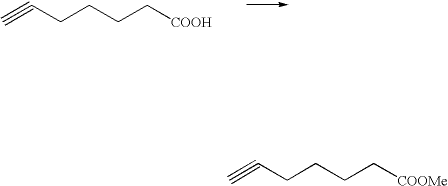

To a solution of hept-6-ynoic acid (2.0 g, 15.9 mmol) in MeOH (4 ml), conc. H₂SO₄ (4 drops) was added at room temperature and stirred at 60 degrees C. for 60 h. To the mixture, ethyl acetate was added and the mixture was washed with saturated NaHCO₃, dried over MgSO₄ and concentrated in vacuo to give the title compound (2.0 g, 90%).

¹H-NMR: 1.52-1.61 (m, 2H), 1.68-1.80 (m, 2H), 1.95-1.97 (m, 1H), 2.18-2.23 (m, 2H), 2.35 (t, 2H), 3.68 (s, 3H).

### (2) Preparation of 7-(4-{1-Ethyl-1-[3-methyl-4-(4,4,4-trifluoro-3-methoxymethoxy-3-trifluoromethyl-but-1-ynyl)-phenyl]-propyl}-2-methyl-phenyl)-hept-6-ynoic acid methyl ester

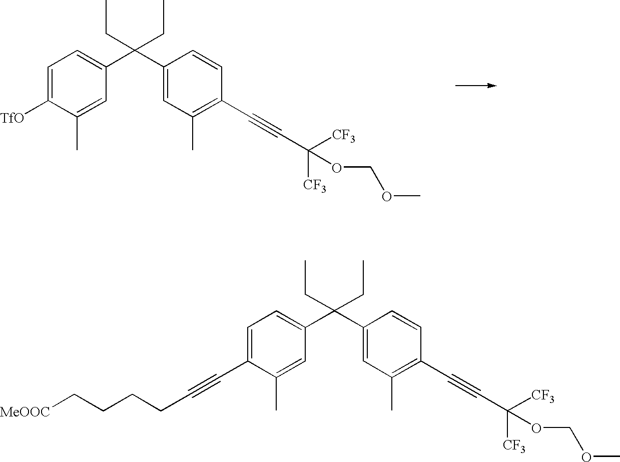

To a solution of trifluoro-methanesulfonic acid 4-{1-ethyl-1-[3-methyl-4-(4,4,4-trifluoro-3-methoxymethoxy-3-trifluoromethyl-but-1-ynyl)-phenyl]-propyl}-2-methyl-phenyl ester (compound prepared in Example 192-(1)) (23 mg, 0.0363 mmol) in DMF (0.5 ml), Et₃N (0.0253 ml, 0.182 mmol), Cul (3.4 mg, 0.0181 mmol), PdCl₂(PPh₃)₂ (12.7 mg, 0.0181 mmol) and hept-6-ynoic acid methyl ester (15.3 mg, 0.109 mmol) were added at room temperature and the mixture was stirred at 160 degrees C. for 30 min. To the mixture, ethyl acetate was added and the mixture was washed with H₂O, dried over MgSO₄, concentrated in vacuo and purified by preparative TLC (n-hexane:ethyl acetate=5:1) to give the title compound (4.0 mg, 18%).

¹H-NMR: 0.59 (t, 6H), 2.08 (q, 4H), 2.35 (s, 3H), 2.38 (s, 3H), 2.47 (t, 2H), 3.47 (s, 2H), 3.67 (s, 3H), 5.15 (s, 2H), 6.87-7.38 (m, 6H).

### (3) Preparation of 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid methyl ester

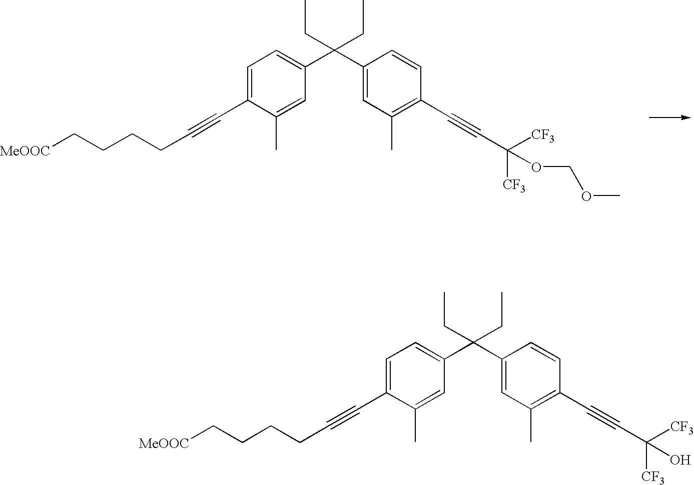

7-(4-{l -Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid methyl ester (compound prepared in Example 193-(2)) (4.0 mg, 0.0064 mmol) was dissolved in 4N HCl/dioxane (0.5 ml) at room temperature, the mixture was stirred at room temperature for 16 h. The mixture was concentrated in vacuo and purified by preparative TLC (n-hexane:ethyl acetate=5:1) to give the title compound (2.6 mg, 70%).

¹H-NMR: 0.60 (t, 6H), 1.85-1.91 (m, 2H), 2.05 (q, 4H), 2.35 (s, 3H), 2.37 (s, 3H), 2.47 (t, 2H), 3.67 (s, 3H), 6.83-7.37 (m, 6H); MS (ESI+): 598 ([M+NH₄]⁺).

### (4) Preparation of 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid

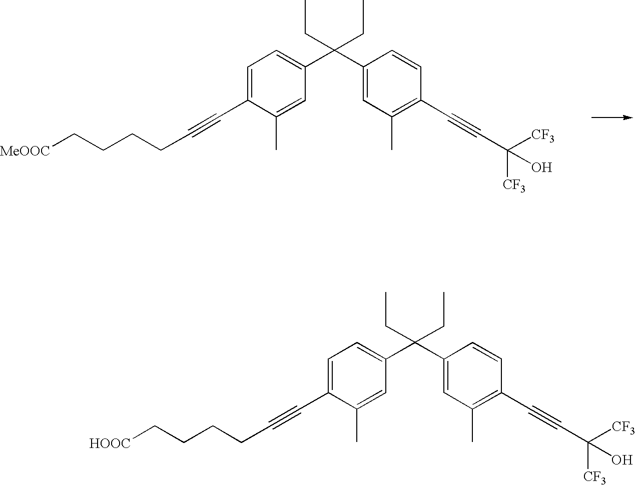

To a solution of 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid methyl ester (2.6 mg, 0.0045 mmol) in MeOH (0.5 ml), 1N NaOH (0.1 ml) was added and the mixture was stirred at room temperature for 1 h. The mixture was concentrated in vacuo and purified by preparative TLC (CHCl₃:MeOH=8:3 saturated with H₂O) to give the title compound (1.0 mg, 40%).

¹H-NMR: 0.59 (t, 6H), 1.79-1.89 (m, 2H), 2.06 (q, 4H), 2.35 (s, 3H), 2.37 (s, 3H), 2.42-2.50 (m, 2H), 6.85-7.35 (m, 6H); MS (ESI+): 584 ([M+NH₄]⁺).

### Example 194

### Preparation of 5-(4-{1-Ethyl-1-[4-(3-hydroxy-4,4-dimethyl-pentyl)-3-methyl-phenyl]-propyl}-2-furan-2-carboxylic acid

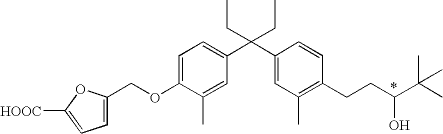

### (1) Preparation of 5-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-furan-2-carboxylic acid ethyl ester

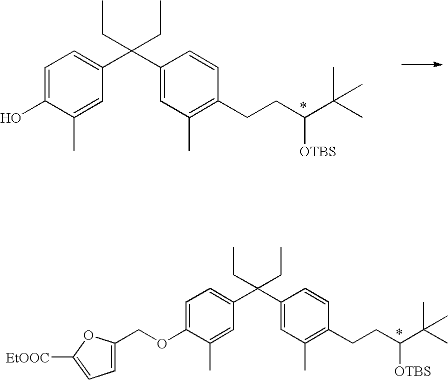

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 187-(1)) (60 mg, 0.12 mmol) in DMF (0.6 ml), ethyl 5-(chloromethyl)-2-furan carboxylate (27 mg, 0.14 mmol), and K₂CO₃ (20 mg, 0.14 mmol) were added at room temperature and stirred at 80 degrees C. for 8 h. To the mixture, ethyl acetate was added and the mixture was washed with saturated NH₄Cl solution and water, dried over MgSO₄, filtered and concentrated in vacuo. The obtained residue was chromatographed on silica gel (ethyl acetate/hexane=1/4) to give the title compound (60 mg, 93%) as colorless oil.

¹H-NMR (CDCl₃): 0.06 (s, 3H), 0.1 (s, 3H), 0.6 (t, 6H, J=7.3 Hz), 0.88 (s, 9H), 0.93 (s, 9H), 1.38 (t, 3H, J=7.2 Hz), 1.52-1.64 (m, 1H), 1.71-1.82 (m, 1H), 2.04 (q, 4H, J=7.3 Hz), 2.17 (s, 3H), 2.23 (s, 3H), 2.41 (dt, 1H, J=4.7, 13.2 Hz), 2.76 (dt, 1H, J=5.4, 12.8 Hz), 3.34 (dd, 1H, J=3.3, 7.3 Hz), 4.37 (q, 2H, J=7.2 Hz), 6.49 (d, 1H, J=3.5 Hz), 6.73 (d, 1H, J=9.1 Hz), 6.9-7.0 (m, 5H), 7.15 (d, 1H, J=3.5 Hz).

### (2) Preparation of 5-(4-{1-Ethyl-1-[4-(3-hydroxy-4,4-dimethyl-pentyl)-3-methyl-phenyl]-propyl}-2-methyl -phenoxymethyl)-furan-2-carboxylic acid

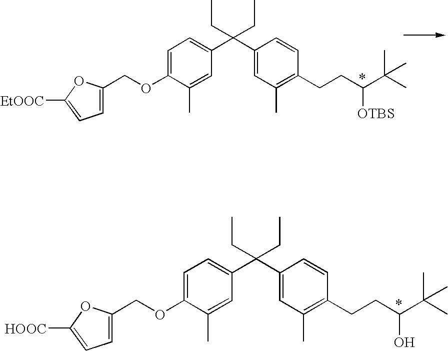

To a solution of 5-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-furan-2-carboxylic acid ethyl ester (compound prepared in Example 194-(1)) (60 mg, 0.11 mmol) in THF (1 ml), TBAF (1.0 M in THF) (1 ml, 1 mmol) was added at room temperature and refluxed for 5 h. To the mixture, ethyl acetate was added and the mixture was washed with 30% NaH₂PO₄ aq. and brine, dried over MgSO₄, filtered and concentrated in vacuo. The obtained residue was chromatographed on silica gel (MeOH/CH₂Cl₂=1/10) to give the title compound (25 mg, 45%) as colorless oil.

¹H-NMR (CDCl₃): 0.59 (t, 6H, J=7.3 Hz), 0.89 (s, 9H), 1.43-1.58 (m, 1H), 1.74-1.82 (m, 1H), 2.03 (q, 4H, J=7.3 Hz), 2.16 (s, 3H), 2.25 (s, 3H), 2.5-2.61 (m, 1H), 2.81-2.92 (m, 1H), 3.25 (dd, 1H, J=1.6, 10.4 Hz), 4.06 (brs, 1H), 5.04 (s, 2H), 6.5-6.49 (d, 1H, J=3.5 Hz), 6.73-6.70 (d, 1H, J=5.5 Hz), 6.89-6.95 (m, 4H), 7.02 (d, 1H, J=8.6 Hz), 7.25 (d, 1H, J=4.3 Hz); MS (ESI+): 529 ([M+Na]⁺).

### Example 195

### Preparation of Preparation of (E)-N-(2-Amino-ethyl)-2-(4-{I -ethyl-1-[4-(3-ethyl-3-hydroxy-pent-1-enyl)-3-methyl-phenyl]-propyl}-2-methyl-phenoxy)-acetamide

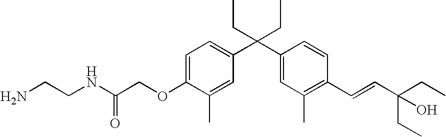

### (1) Preparation of (E)-{2-[2-(4-{1-Ethyl-1-[4-(3-ethyl-3-hydroxy-pent-1-enyl)-3-methyl-phenyl]-propyl}-2-methyl-phenoxy)-acetylamino]-ethyl}-carbamic acid 9H-fluoren-9-ylmethyl ester

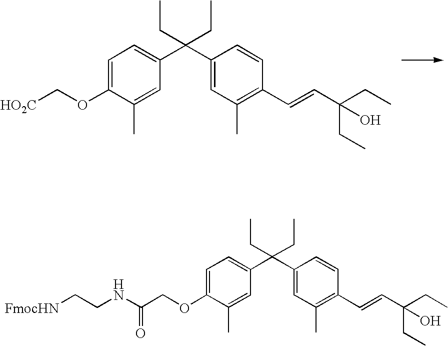

To a solution of (4-{1-Ethyl-1-[4-(3-ethyl-3-hydroxy-pent-1-enyl)-3-methyl-phenyl]-propyl)-2-methyl-phenoxy)-acetic acid (compound prepared in Example 151-(2)) (74.4 mg, 0.17 mmol) in CH₂Cl₂ (1.7 ml) and diisopropylethylamine (0.118 ml, 0.68 mmol) was added (2-Amino-ethyl)-carbamic acid 9H-fluoren-9-ylmethyl ester (108 mg, 0.34 mmol), WSCI hydrochloride (65 mg, 0.34 mmol) and HOBt monohydrate (26 mg, 0.17 mmol), and the mixture was stirred for 18 h at room temperature. The reaction mixture was poured into H₂O and extracted with CH₂Cl₂. The organic phase was concentrated in vacuo. The resulting residue was chromatographed on silica gel (ethyl acetate/hexane=60/40 to 100/0) to give the title compound (92.2 mg, 77%).

¹H-NMR: 0.59 (t, 6H), 0.91 (t, 6H), 1.63 (dq, 4H), 2.03 (q, 4H), 2.21 (s, 3H), 2.30 (s, 3H), 3.30-3.55 (m, 4H), 4.19 (t, 1H), 4.38 (d, 2H), 4.47 (s, 2H), 5.16 (br, 1H), 6.00 (d, 1H), 6.62 (d, 1H), 6.73 (d, 1H), 6.90-7.75 (m, 14H).

### (2) Preparation of (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide

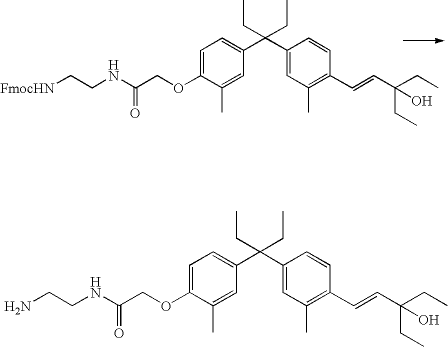

To a solution of {2-[2-(4-{1-Ethyl-1-[4-(3-ethyl-3-hydroxy-pent-1-enyl)-3-methyl-phenyl]-propyl}-2-methyl-phenoxy)-acetylamino]-ethyl}-carbamic acid 9H-fluoren-9-ylmethyl ester (92 mg, 0.13 mmol) in DMF (1.3 ml), diethylamine (0.134 ml, 1.3 mmol) was added and the mixture was stirred for 3 h at room temperature. The reaction mixture was poured into water and the products were extracted with AcOEt. The extracts were washed with H₂O twice, dried over MgSO₄, filtered and concentrated in vacuo. The obtained residue was chromatographed on silica gel (ethyl acetate/EtOH=10/1) to give the title compound (41.1 mg, 65%).

¹H-NMR: 0.61 (t, 6H), 0.92 (t, 6H), 1.64 (dq, 4H), 2.05 (q, 4H), 2.23 (s, 3H), 2.31 (s, 3H), 2.87 (t, 2H), 3.40 (q, 2H), 4.49 (s, 2H), 6.01 (d, 1H), 6.66 (d, 1H), 6.74 (d, 1H), 6.90-7.75 (m, 6H).

### Example 196

### Preparation of Preparation of (E)-2-(4-{1-Ethyl-1-[4-(3-ethyl-3-hydroxy-pent-1-enyl)-3-methyl-phenyl]-propyl}-2-methyl-phenoxy)-N-(2-hydroxy-ethyl)-acetamide

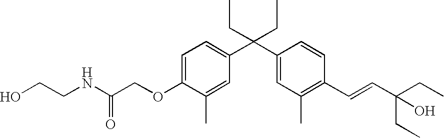

### Preparation of (E)-2-(4-{1-Ethyl-1-[4-(3-ethyl-3-hydroxy-pent-1-enyl)-3-methyl-phenyl]-propyl}-2-methyl-phenoxy)-N-(2-hydroxy-ethyl)-acetamide

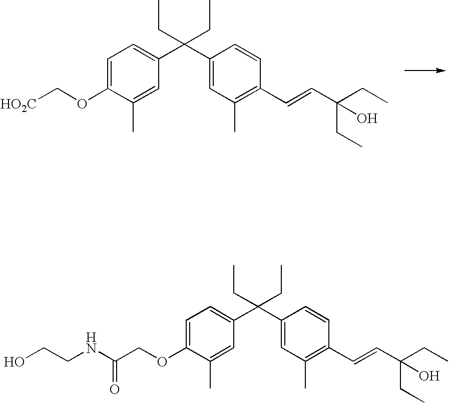

To a solution of (4-{1-Ethyl-1-[4-(3-ethyl-3-hydroxy-pent-1-enyl)-3-methyl-phenyl]-propyl}-2-methyl-phenoxy)-acetic acid (compound prepared in Example 151-(2)) (78 mg, 0.178 mmol) in CH₂Cl₂ (1.8 ml) and triethylamine (0.074 ml, 0.534 mmol) was added ethanolamine (0.032 ml, 0.534 mmol), WSCI hydrochloride (102 mg, 0.534 mmol) and HOBt monohydrate (27 mg, 0.178 mmol), and the mixture was stirred for 2 h at room temperature. To the reaction mixture, H₂O was added and separated. The organic phase was concentrated in vacuo. The resulting residue was chromatographed on silica gel (ethyl acetate/hexane=60/40 to 100/0) to give the title compound (21.5 mg, 25%).

¹H-NMR: 0.61 (t, 6H), 0.92 (t, 6H), 1.64 (dq, 4H), 2.05 (q, 4H), 2.22 (s, 3H), 2.31 (s, 3H), 3.54 (q, 2H), 3.76-3.79 (m, 2H), 4.50 (s, 2H), 6.01 (d, 1H), 6.65-7.31 (m, 8H); MS (ESI+): 464 ([M−OH]⁺).

### Example 197

### Preparation of [2-(4-{1-Ethyl-1-[4-((E)-3-ethyl-3-hydroxy-pent-1-enyl)-3-methyl-phenyl]-propyl}-2-methyl-phenoxy)-acetylamino]-acetic acid

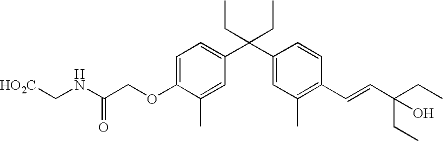

### Preparation of [2-(4-{1-Ethyl-1-[4-((E)-3-ethyl-3-hydroxy-pent-1-enyl)-3-methyl-phenyl]-propyl}-2-methyl-phenoxy)-acetylamino]-acetic acid

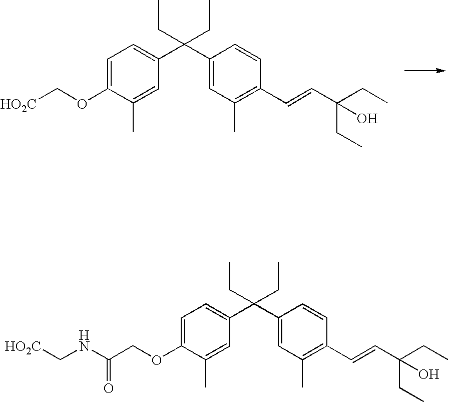

To a solution of (4-{1-Ethyl-1-[4-(3-ethyl-3-hydroxy-pent-1-enyl)-3-methyl-phenyl]-propyl}-2-methyl-phenoxy)-acetic acid (compound prepared in Example 151-(2)) ( (172 mg, 0.39 mmol) in CH₂Cl₂ (3.9 ml) and triethylamine (0.231 ml, 1.66 mmol) was added H-Gly-OMe hydrochloride (98 mg, 0.78 mmol), WSCI hydrochloride (150 mg, 0.78 mmol) and HOBt monohydrate (60 mg, 0.39 mmol), stirred for 18 h at room temperature. The reaction mixture was concentrated in vacuo, separated with ethyl acetate and aqueous KHSO₄, the organic phase was washed with aqueous NaHCO₃ and brine, dried over MgSO₄, concentrated in vacuo to obtain the residue (174 mg). Then, 96.8 mg of the residue was dissolved with THF (1 ml) and MeOH (1 ml), 1N aqueous KOH (1 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo, and chromatographed on silica gel (MeOH/CHCl₃=0/100 to 27/73) to give the title compound (87 mg, 81%).

¹H-NMR (CD₃OD): 0.59 (t, 6H), 0.91 (t, 6H), 1.62 (q, 4H), 2.06 (q, 4H), 2.22 (s, 3H), 2.26 (s, 3H), 3.84 (s, 2H), 4.49 (s, 2H), 5.98 (d, 1H), 6.71-6.99 (m, 7.27 (d, 1H); MS (EI): 477 ([M−H₂O]⁺).

### Example 198

### Preparation of (E)-5-(4-{1-Ethyl-1-[4-(3-ethyl-3-hydroxy-pent-1-enyl)-3-methyl-phenyl]-propyl)-2-methyl-phenoxy)-4-oxo-pentanoic acid

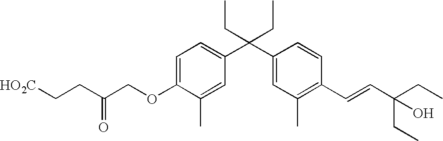

### (1) Preparation of (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid methyl ester

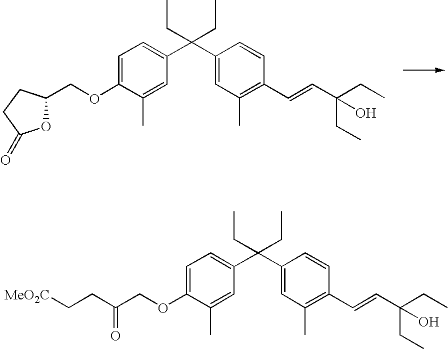

To a solution of 5-(4-{1-Ethyl-1-[4-(3-ethyl-3-hydroxy-pent-1-enyl)-3-methyl-phenyl]-propyl)-2-methyl-phenoxymethyl)-dihydro-furan-2-one (compound prepared in Example 44) (194 mg, 0.41 mmol) was dissolved with THF (1.5 ml) and MeOH (1.5 ml). 1N aqueous KOH (1.5 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo. The resulting residue was dissolved with CH₂Cl₂ (0.8 ml), tetrapropylammonium perruthenate (14.4 mg, 0.041 mmol), MS4A (200 mg), and N-methylmorphorine-N-oxide (96 mg, 0.82 mmol) was added and stirred for 2 h at room temperature. The reaction mixture was concentrated in vacuo and treated with short pad of silica gel (ethyl acetate/EtOH=10/1). The resulting black residue was dissolved with Et₂O (2 ml) and MeOH (1 ml). Trimethylsilyldiazomethane (2M in hexane, ca. 0.2 ml), was added and stirred for 2 min. at room temperature. The reaction mixture was concentrated in vacuo and chromatographed (ethyl acetate/hexane=15/85 to 30/70) to give the title compound (65 mg, 32%).

¹H-NMR: 0.61 (t, 6H), 0.91 (t, 6H), 1.64 (q, 4H), 2.05 (q, 4H), 2.24 (s, 3H), 2.31 (s, 3H), 2.66 (t, 2H), 2.99 (t, 2H), 3.68 (s, 3H), 4.55 (s, 2H), 6.01 (d, 1H), 6.56 (d, 1H), 6.74 (d, 1H), 6.92-6.96 (m, 4H), 7.30 (d, 1H).

### (2) Preparation of (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid

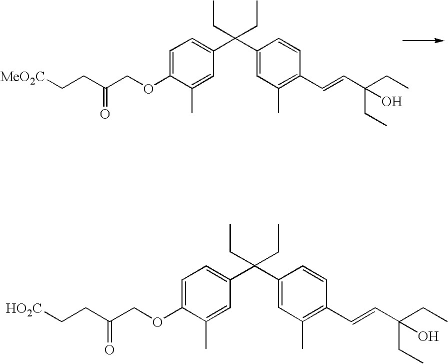

To a solution of 5-(4-{1-Ethyl-1-[4-(3-ethyl-3-hydroxy-pent-1-enyl)-3-methyl-phenyl]-propyl}-2-methyl-phenoxy)-4-oxo-pentanoic acid methyl ester (compound prepared in Example 198-(1)) (65 mg, 0.41 mmol) was dissolved with THF (1 ml) and MeOH (1 ml). 1N. aqueous KOH (1 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo to give the title compound (63 mg, 100%).

¹H-NMR(CD₃OD): 0.61 (t, 6H), 0.91 (t, 6H), 1.62 (q, 4H), 2.06 (q, 4H), 2.19 (s, 3H), 2.26 (s, 3H), 2.59-2.63 (m, 2H), 2.84-2.88 (m, 2H), 5.98 (dd, 1H), 6.64 (d, 1H), 6.74 (d, 1H), 6.89-6.95 (m, 4H), 7.27 (d, 1H); MS (El): 476 ([M−H₂O]⁺).

### Example 199

### Preparation of (E)-3-[2-(4-{1-Ethyl-1-[4-(3-ethyl-3-hydroxy-pent-1-enyl)-3-methyl-phenyl]-propyl}-2-methyl-phenoxy)-acetylamino]-propionic acid

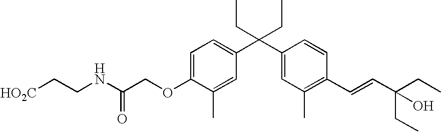

### Preparation of (E)-3-[2-(4-{1-Ethyl-1-[4-(3-ethyl-3-hydroxy-pent-1-enyl)-3-methyl-phenyl]-propyl}-2-methyl-phenoxy)-acetylamino]-propionic acid

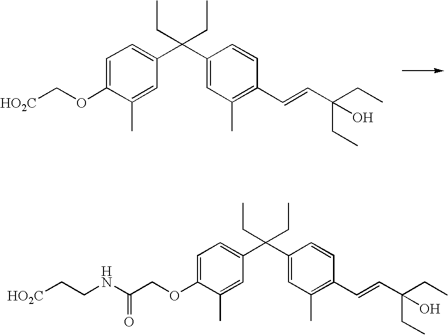

To a solution of (4-{1-Ethyl-1-[4-(3-ethyl-3-hydroxy-pent-1-enyl)-3-methyl-phenyl]-propyl}-2-methyl-phenoxy)-acetic acid (compound prepared in Example 151-(2)) ( (166 mg, 0.38 mmol) in CH₂Cl₂ (3.8 ml).and triethylamine (0.212 ml, 1.52 mmol) was added H-beta-Ala-OMe hydrochloride (106 mg, 0.76 mmol), WSCI hydrochloride (146 mg, 0.76 mmol) and HOBt monohydrate (58 mg, 0.38 mmol), and the mixture was stirred for 18 h at room temperature. The reaction mixture was concentrated in vacuo, separated with ethyl acetate and aqueous KHSO₄, the organic phase was washed with aqueous NaHCO₃ and brine, dried over MgSO₄, concentrated in vacuo to obtain the residue (162 mg). Then, 103.6 mg of the residue was dissolved with THF (1 mL) and MeOH (1 ml). 1N aqueous KOH (1 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo, and chromatographed on silica gel (MeOH/CHCl₃=0/100 to 27/73) to give the title compound (78 mg, 63%).

¹H-NMR: 0.60 (t, 6H), 0.91 (t, 6H), 1.63 (dq, 4H), 2.04 (q, 4H), 2.20 (s, 3H), 2.30 (s, 3H), 2.64 (t, 2H), 3.64 (q, 2H), 4.45 (s, 2H), 6.00 (d, 1H), 6.63 (d, 1H), 6.37 (d, 1H), 6.91-7.30 (m, 5H); MS (EI): 491 ([M−H₂O]⁺).

### Example 200

### Preparation of (2S, 3S)-2-[2-(4-{1-Ethyl-1-[4-(3-hydroxy-4,4-dimethyl-pentyl)-methyl-phenyl]-propyl}-2-methyl-phenoxy)-acetylamino]-3-methyl-pentanoic acid

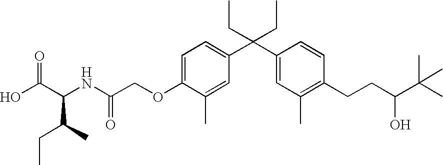

### (1) Preparation of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid methyl ester

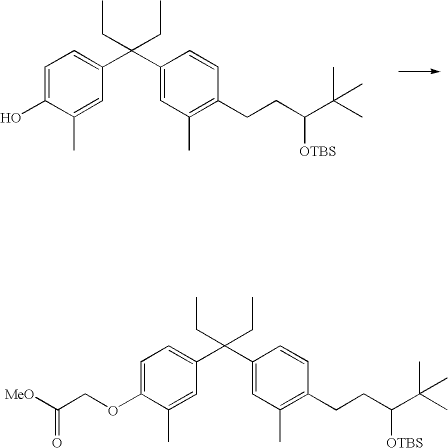

To a solution of compound prepared in Example 186-(1) (4-(1-{4-[3-(tert-Butyl-dimethyl-silanyloxy)-4,4-dimethyl-pentyl]-3-methyl-phenyl}-1-ethyl-propyl)-2-methyl-phenol) (450 mg, 0.91 mmol) in acetone (10 ml), K₂CO₃ (377 mg, 2.73 mmol) and bromoacetic acid methyl ester (278 mg, 1.82 mg) were added at room temperature and the mixture was stirred at 45 degrees C. for 68 h. The mixture was applied onto silica and chromatographed (n-hexane only to n-hexane:ethyl acetate=5:1) to give the title compound (436 mg, 84%).

¹H-NMR: 0.07 (s, 3H), 0.11 (s, 3H), 0.60 (t, 6H), 0.89 (s, 9H), 0.94 (s, 9H), 2.03 (s, 4H), 2.24 (s, 6H), 2.35-2.46 (m, 1H), 2.70-2.82 (m, 1H), 3.33-3.38 (m, 1H), 3.80 (s, 3H), 4.62 (s, 2H), 6.59 (d, 1H), 6.88-7.00 (m, 5H).

### (2) Preparation of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid

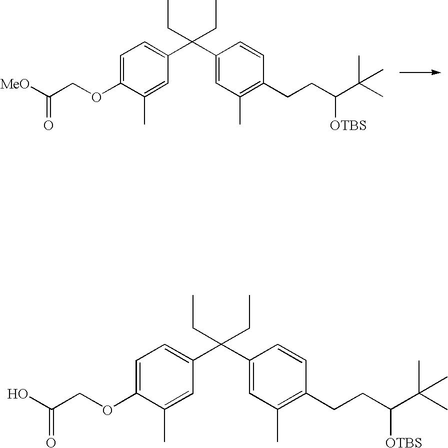

To a solution of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid methyl ester (436 mg, 0.767 mmol) in EtOH (3 ml), 1N NaOH (1 ml) was added at room temperature and the mixture was stirred at room temperature for 16 h. To the mixture, 1% HCl was added to adjust pH at 6 and extracted with ethyl acetate, washed with brine, dried over MgSO₄ and concentrated in vacuo to give the title compound (424 mg, 100%).

¹H-NMR: 0.07 (s, 3H), 0.10 (s, 3H), 0.60 (t, 6H), 0.88 (s, 9H), 0.94 (s, 9H), 2.03 (s, 4H), 2.23 (s, 6H), 2.35-2.47 (m, 1H), 2.70-2.82 (m, 1H), 3.33-3.38 (m, 1H), 4.61 (s, 2H), 6.62 (d, 1H), 6.88-7.02 (m, 5H).

### (3) Preparation of (2S,3S)-2-[2-(4-{1-Ethyl-1-[4-(3-hydroxy-4,4-dimethyl-pentyl)-3-methyl-phenyl]-propyl}-2-methyl-phenoxy)-acetylamino]-3-methyl-pentanoic acid

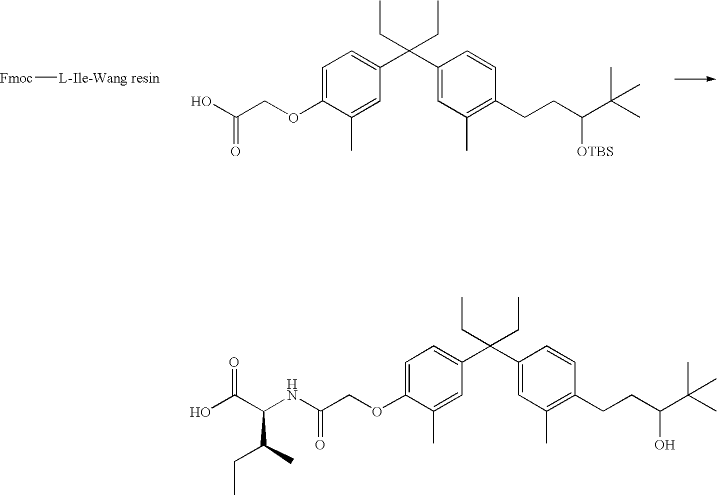

Fmoc-L-Ile-Wang resin (0.57 mmol/g as amino acid loading, 35 mg, 0.020 mmol amino acid loading) was treated with 20% (v/v) piperidine in DMF (0.5 ml) two times to deprotect Fmoc group and washed with DMF (1 ml, 5 times). To the resin, solutions of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid (16.0 mg, 0.029 mmol) in DMF (0.2 ml), benzotriazole-1-yl-oxy-tris(dimethylamino)phosphonium hexafluorophosphate (17.7 mg, 0.040 mmol) in DMF (0.2 ml) and N,N-diisopropyl ethyl amine (0.0105 ml, 0.060 mmol) in DMF (0.1 ml) were added at room temperature, agitated at room temperature for 16 h, filtrated, washed with DMF (2 ml, 3 times), i-PrOH (2 ml, 3 times), THF (2 ml, 3 times), MeOH (2 ml, 3 times) and CH₂Cl₂ (2 ml, 3 times) and dried in vacuo. To the resin, 20% TFA in CH₂Cl₂ (v/v) (1 ml) was added at room temperature, agitated at room temperature for 16 h, filtrated, washed with CH₂Cl₂ (1 ml, 2 times) and concentrated in vacuo. The residue was purified by preparative reverse phase HPLC (CAPCELLPAK C18 MG2 20 mm(ID)-50 mm column, gradient elution 50 to 90% B/A over 10 min, and 100% B over 2.5 min (solvent A=10 mM ammonium acetate H₂O, solvent B=MeOH, UV detection at 220 and 277 nm) to give the titled compound (3.4 mg, 31%).

¹H-NMR: 0.59 (t, 6H), 0.89 (s, 9H), 0.89-0.97 (m, 6H), 2.04 (q, 4H), 2.23 (s, 3H), 2,25 (s, 3H), 3.25 (d, 1H), 4.51 (s, 2H), 4.60-4.68 (m, 1H), 5.30 (s, 2H), 6.65 (d, 1H), 6.88-7.05 (m, 4H), 7.12 (d, 1H); MS (ESI+): 571 ([M+NH₄]⁺).

### Example 201-202

Examples 201 to 202 were prepared according to same procedure as Example 200-(3) from corresponding Fmoc-amino acid-Wang resin listed in below table.

| Example | | | ¹H-NMR in | | yield | yield |
|---|---|---|---|---|---|---|
| # | compound | structure | CD₃OD | MS | (mg) | (%) |
| 201 | 2(S)-12-(4-{1- Ethyl-1-[4-(3- hydroxy-4,4- dimethyl- pentyl)-3- methyl-phenyl]- propyl}-2- methyl- phenoxy)- acetylamino}- succinic acid | 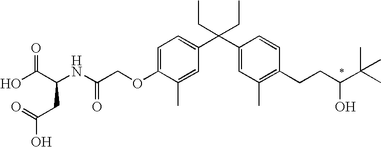 | 0.58 (t, 6H), 0.89 (t, 9H), 2.04 (q, 4H), 2.25 (s, 6H), 3.15 (d, 1H), 4.50 (s, 2H), 4.63 (t, | | | |
| # 1H), 6.72 (d, 1H), 6.87-7.04 (m, 1H) | 573 ([M + NH₄]⁺) | 1.7 | 15 | | | |
| 202 | 3-[2-(4-{1-Ethyl- 1-[4-(3-hydroxy- 4,4-dimethyl- pentyl)-3- methyl-phenyl]- propyl}-2- methyl- phenoxy)- acetylamino]- propionic acid | 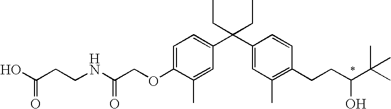 | 0.59 (t, 6H), 0.89 (s, 9H), 2.03 (q, 4H), 2.20 (s, 3H), 2.25 (s, 3H), 2.61 (t, 2H), 3.22 (d, 1H), 3.62 (q, 2H), | | | |
| # 4.46 (s, 2H), 6.71 (d, 1H), 6.87-7.04 (m, 5H) | 529 ([M + NH₄]⁺) | 3.7 | 36 | | | |

Starting Fmoc-amino acid-Wang resin

| | | amino acid | |
|---|---|---|---|
| | | loading on | |
| | | resin | reacted resin |
| Example # | Fmoc-amino acid-Wang resin | (mmol/g) | (mg) |
| 201 | Fmoc-L-Asp(OtBu)-Wang resin | 0.59 | 33.9 |
| 202 | Fmoc-beta-Ala-Wang resin | 0.52 | 38.5 |

### Example 203

### Preparation of [2-(4-{1-Ethyl-1-[4-(3-hydroxy-4,4-dimethyl-pentyl)-3-methyl-phenyl]-propyl}-2-methyl-phenoxy)-acetylamino]-acetic acid

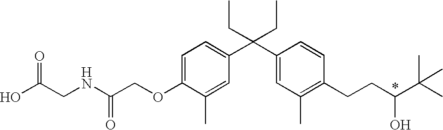

### Preparation of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid methyl ester

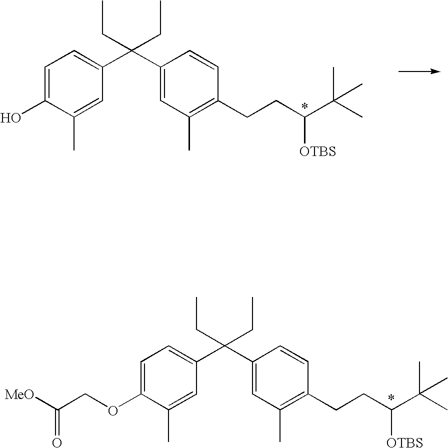

To a solution of compound prepared in Example 187-(1) (4-(1-{4-[3-(tert-Butyl-dimethyl-silanyloxy)-4,4-dimethyl-pentyl]-3-methyl-phenyl}-1-ethyl-propyl)-2-methyl-phenol) (1.00 g, 2.02 mmol) in acetone (20 ml), K₂CO₃ (836 mg, 6.06 mmol) and bromoacetic acid methyl ester (618 mg, 4.04 mmol) was added at room temperature and stirred at 45 degrees C. for 68 h. The mixture was applied onto silica and chromatographed (n-hexane only to n-hexane:ethyl acetate=5:1) to give the title compound (720 mg, 63%).

¹H-NMR: 0.06 (s, 3H), 0.11 (s, 3H), 0.59 (t, 6H), 0.88 (s, 9H), 0.93 (s, 9H), 2.03 (s, 4H), 2.23 (s, 6H), 2.35-2.46 (m, 1H), 2.70-2.82 (m, 1H), 3.34 (dd, 1H), 3.80 (s, 3H), 4.61 (s, 2H), 6.57 (d, 1H), 6.90-6.99 (m, 5H).

### (2) Preparation of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid

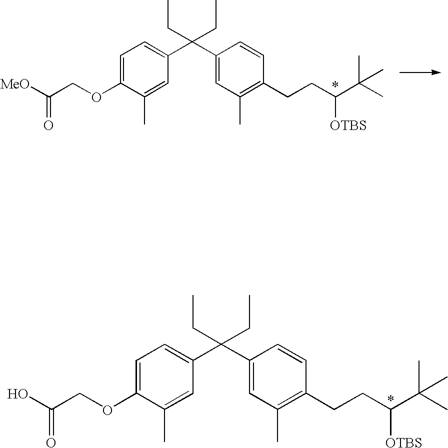

To a solution of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid methyl ester prepared in Example 203-(1) (720 mg, 1.27 mmol) in MeOH (5 ml) and THF (5 ml), 1N NaOH (1 ml) was added at room temperature and the mixture was stirred at room temperature for 16 h. To the mixture, 1% HCl was added to adjust pH at 6 and extracted with ethyl acetate, washed with brine, dried over MgSO₄ and concentrated in vacuo to give the title compound (690 mg, 100%).

¹H-NMR: 0.07 (s, 3H), 0.10 (s, 3H), 0.60 (t, 6H), 0.88 (s, 9H), 0.93 (s, 9H), 2.05 (s, 4H), 2.23 (s, 6H), 2.35-2.46 (m, 1H), 2.70-2.82 (m, 1H), 3.34 (dd, 1H), 4.65 (s, 2H), 6.61 (d, 1H), 6.88-7.02 (m, 5H).

### (3) Preparation of [2-(4-{1-Ethyl-1-[4-(3-hydroxy-4,4-dimethyl-pentyl)-3-methyl-phenyl]-propyl}-2-methyl-phenoxy)-acetylamino]-acetic acid

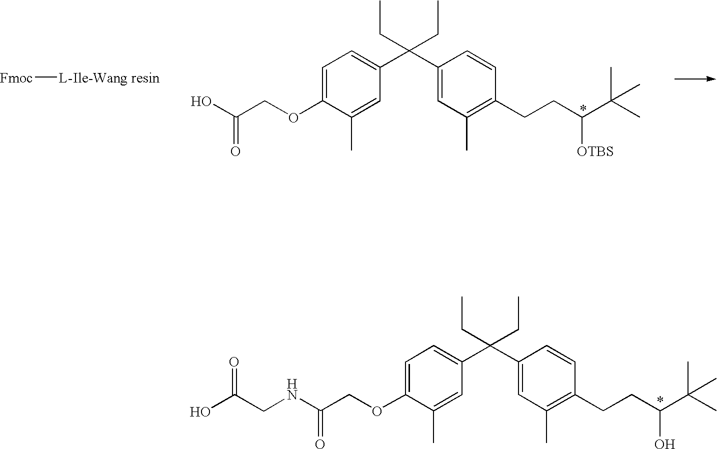

Fmoc-Gly-Wang resin (0.73 mmol/g as amino acid loading, 27.4 mg, 0.020 mmol amino acid loading) was treated with 20% (v/v) piperidine in DMF (0.5 ml) two times to deprotect Fmoc group and washed with DMF (1 ml, 5 times). To the resin, solutions of [4-(1-{4-[3-(tert-Butyl-dimethyl-silanyloxy)-4,4-dimethyl-pentyl]-3-methyl-phenyl}-1-ethyl-propyl)-2-methyl-phenoxy]-acetic acid (16.0 mg, 0.029 mmol) in DMF (0.2 ml), benzotriazole-1-yl-oxy-tris(dimethylamino)phosphonium hexafluorophosphate (17.7 mg, 0.040 mmol) in DMF (0.2 ml) and N,N-diisopropyl ethyl amine (0.0105 ml, 0.060 mmol) in DMF (0.1 ml) were added at room temperature, agitated at room temperature for 16 h, filtrated, washed with DMF (2 ml, 3 times), i-PrOH (2 ml, 3 times), THF (2 ml, 3 times), MeOH (2 ml, 3 times) and CH₂Cl₂ (2 ml, 3 times) and dried in vacuo. To the resin, 20% TFA in CH₂Cl₂ (v/v) (1 ml) was added at room temperature, agitated at room temperature for 16 h, filtrated, washed with CH₂Cl₂ (1 ml, 2 times) and concentrated in vacuo. The residue was purified by preparative reverse phase HPLC (CAPCELLPAK C18 MG2 20 mm(ID)-50 mm column, gradient elution 50 to 90% B/A over 10 min, and 100% B over 2.5 min (solvent A=10 mM ammonium acetate H₂O, solvent B=MeOH, UV detection at 220 and 277 nm) to give the titled compound (2.6 mg, 26%).

¹H-NMR: 0.59 (s, 6H), 0.87 (s, 9H), 2.07 (q, 4H), 2.24 (S, 6H), 3.15 (d, 1H), 3.91 (d, 2H), 4.52 (s, 2H), 6.77 (d, 1H), 6.87-7.04 (m, 5H); MS (ESI+): 515 ([M+NH₄]⁺).

### Example 204-243

Examples 204 to 243 were prepared according to same procedure as Example 203-(3) from corresponding Fmoc-amino acid-Wang resin listed in below table.

| Ex- | | | | | | |
|---|---|---|---|---|---|---|
| am | | | | | | |
| ple | | | ¹H-NMR in | MS(positive | yield | yield |
| # | compound | structure | CD3OD | mode) | (mg) | (%) |
| 204 | 3-(2-(4-(1-Ethyl-1- [4-(3-hydroxy-4,4- dimethyl-pentyl)-3- methyl-phenyl]- propyl}-2-methyl- phenoxy)- acetylamino}- propionic acid | 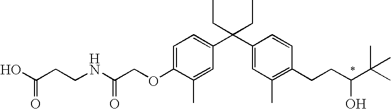 | 0.59 (s, 6H), 0.87 (s, 9H), 2.07 (q, 4H), 2.19 (s, 3H), 2.22 (s, 3H), 2.43 (t, 2H), 3.15 (d, 1H), 3.52 (t, 2H), 4.47 (s, 2H), | | | |
| # 6.72 (d, 1H), 6.88-7.05 (m, 5H) | 529 [M + NH₄]⁺ | 1.0 | 10 | | | |
| 205 | 4-[2-(4-{1-Ethyl-1- [4-(3-hydroxy-4,4- dimethyl-pentyl)-3- methyl-phenyl]- phenoxy)- acetylainino}- butync acid | 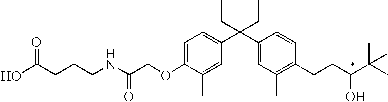 | 0.63 (t, 6H), 0.91 (s, 9H), 2.08 (q, 4H), 2.26 (s, 3H), 2.28 (s, 3H), 3.19 (d, 1H), 4.52 (s, 2H), 6.75 (d, 1H), 6.90-7.07 (m, 5H) | | | |
| # 543 [M + NH₄]⁺ | 2.1 | 20 | | | | |
| 206 | 2(S)-[2-(4-{1-Ethyl- 1-{4-(3-hydroxy- 4,4-dimethyl- pentyl)-3-methyl- methyl-phenoxy)- acetylamino}- propionic acid | 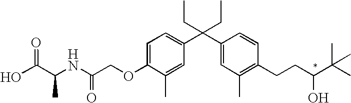 | 0.59 (t, 6H), 0.87 (s, 9H), 1.42 (d, 3H), 2.04 (q, 4H), 2.22 (s, 3H), 2.24 (s, 3H), 3.18 (d, 1H), 4.28 (q, 1H), 4.48 (s, 2H), 6.75 (d, 1H), | | | |
| # 6.89-7.05 (m, 5H) | 529 [M + NH₄]⁺ | 1.2 | 12 | | | |
| 207 | 2(R)-(2-(4-(1-Ethyl- 1-[4-(3-hydroxy- 4,4-dimethyl- pentyl)-3-methyl- phenyl}-propyl}-2- methyl-phenoxy)- acetylamino]- propionic acid | 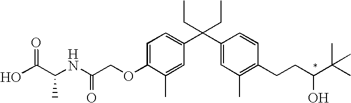 | 0.62 (t, 6H), 0.91 (s, 9H), 1.48 (d, 3H), 2.10 (q, 4H), 2.25 (s, 3H), 2.26 (s, 3H), 3.19 (d, 1H), 4.52 (q, 1H), | | | |
| # 4.56 (s, 2H), 6.80 (d, 1H), 6.92-7.05 (m, 5H) | 529 [M + NH₄]⁺ | 1.7 | 17 | | | |
| 208 | 2(S)-(2-(4-{1-Ethyl- 1-(4-(3-hydroxy- 4,4-dimethyl- pentyl)-3-methyl- phenyl}-propyl}-2- methyl-phenoxy)- acetylamino}- butyric acid | 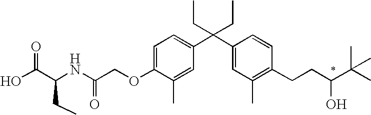 | 0.59 (t, 6H), 0.84 (t, 3H), 0.87 (s, 9H), 2.06 (q, 4H), 2.24 (s, 3H), 2.25 (s, 3H), 3.17 (d, 1H), 4.34 (t, 1H), | | | |
| # 4.52 (s, 2H), 6.77 (d, 1H), 6.89-7.04 (m, 5H) | 543 [M + NH₄]⁺ | 1.0 | 10 | | | |
| 209 | 2(S)-[2-(4-{1-Ethyl- 1-[4-(3-hydroxy- 4,4-dimethyl- pentyl)-3-methyl- methyl-phenoxy)- acetylamino]- pentanoic acid | 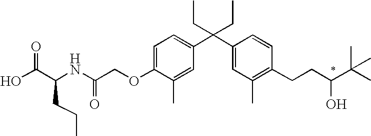 | 0.59 (t, 6H), 0.86 (s, 9H), 0.87 (t, 3H), 2.05 (q, 4H), 3.17 (s, 1H), 4.35 (d, 1H), 4.52 (d, 2H), 6.75 (d, 1H), 6.88-7.04 (m, 5H) | | | |
| # 557 [M + NH₄]⁺ | 2.2 | 20 | | | | |
| 210 | 2(R)-[2-{4-{1-Ethyl- 1-[4-(3-hydroxy- 4,4-dimethyl- pentyl)-3-methyl- phenyl]-propyl}-2- methyl-phenoxy)- acetylamino}- pentanoic acid | 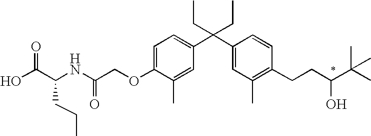 | 0.59 (t, 6H), 0.87 (s, 9H), 0.88 (t, 3H), 2.07 (q, 4H), 2.25 (s, 3H), 2.24 (s, 3H), 3.15 (s, 1H), | | | |
| # 4.34 (t, 1H), 4.50 (d, 2H), 6.74 (d, 1H), 6.88-7.04 (m, 5H) | 557 [M + NH₄]⁺ | 0.6 | 6 | | | |
| 211 | 2(S)-[2-(4-(1-Ethyl- 1-(4-(3-hydroxy- 4,4-dimethyl- pentyl)-3-methyl- phenyl]-propyl}-2- methyl-phenoxy)- acetylamino]- hexanoic acid | 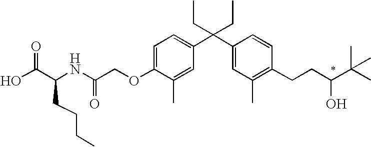 | 0.63 (t, 6H), 0.91 (s, 9H), 0.92 (t, 3H), 2.11 (q, 4H), 2.28 (s, 6H), 3.20 (d, 1H), 4.45 (dd, 1H), 4.57 (d, 2H), 6.78 (d, 1H), | | | |
| # 6.92-7.07 (m, 5H) | 571 [M + NH₄]⁺ | 1.2 | 11 | | | |
| 212 | 2(R)-(2-(4-{1-Ethyl- 1-[4-(3-hydroxy- 4,4-dimethyl- pentyl)-3-methyl- phenyl]-propyl}-2- methyl-phenoxy)- acetylamino}- hexanoic acid | 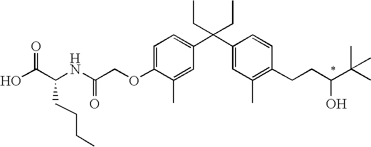 | 0.59 (t, 6H), 0.87 (s, 9H), 0.87 (t, 2H), 2.06 (q, 4H), 2.24 (s, 3H), 2.25 (s, 3H), 3.15 (d, 1H), 4.35 (t, 1H), 4.51 (d, 2H), | | | |
| # 6.74 (d, 1H), 6.88-7.05 (m, 5H) | 571 [M + NH₄]⁺ | 0.4 | 4 | | | |
| 213 | 2(S)-[2-(4-(1-Ethyl- 1-(4-(3-hydroxy- 4,4-dimethyl- pentyl)-3-methyl- phenyl]-propyl}-2- methyl-phenoxy)- acetylamino}-3- methyl-butyric acid | 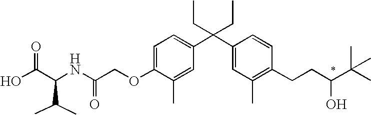 | 0.57 (t, 6H), 0.86-0.90 (m, 6H), 0.88 (s, 9H), 2.05 (q, 4H), 2.22 (s, 3H), 2.23 (s, 3H), 3.14 (d, | | | |
| # 1H), 4.34 (d, 1H), 4.54 (s, 2H), 6.75 (d, 1H), 6.90-7.02 (m, 5H) | 557 [M + NH₄]⁺ | 1.3 | 12 | | | |
| 214 | 2(R)-[2-(4-{1-Ethyl- 1-[4-(3-hydroxy- 4,4-dimethyl- pentyl)-3-methyl- phenyl]-propyl}-2- methyl-phenoxy)- acetylamino]-3- methyl-butyric acid | 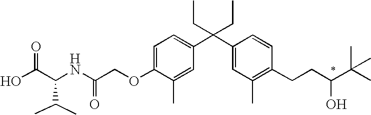 | 0.59 (t, 6H) 0.81-0.91 (m, 6H), 2.06 (q, 4H), 2.24 (s, 3H), 2.26 (s, 3H), 3.16 (d, 1H), 4.31 (d, 1H), | | | |
| # 4.55 (d, 1H), 6.76 (d, 1H), 6.89-7.03 (m, 5H) | 557 [M + NH₄]⁺ | 0.4 | 4 | | | |
| 215 | (2S,3S)-2-[2-(4-(1- Ethyl-1-[4-(3- hydroxy-4,4- dimethyl-pentyl)-3- methyl-phenyl]- propyl}-2-methyl- phenoxy)- acetylamino]-3- methyl-pentanoic acid | 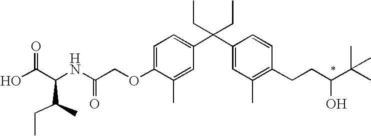 | 0.63 (t, 6H), 0.90-0.97 (m, 6H), 2.08 (q, 4H), 2.28 (s, 3H), 3.20 (s, 1H), 4.49 (d, 1H), | | | |
| # 4.61 (s, 2H), 6.79 (d, 1H), 6.93-7.07 (m, 5H) | 571 [M + NH₄]⁺ | 0.7 | 6 | | | |
| 216 | (2R,3R)-2-[2-(4-{1- Ethyl-1-{4-(3- hydroxy-4,4- dimethyl-pentyl)-3- methyl-phenyl]- propyl}-2-methyl- phenoxy)- acetylamino}-3- methyl-pentanoic acid | 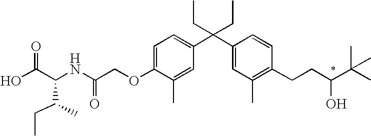 | 0.59 (t, 6H), 0.86-0.90 (m, 15H), 2.06 (q, 4H), 2.24 (s, 3H), 2.25 (s, 3H), 3,16(d, 1H), 4.57 | | | |
| # (d, 2H), 6.75 (d, 1H), 6.89-7.03 (m, 5H) | 571 [M + NH₄]⁺ | 0.4 | 4 | | | |
| 217 | 2(S)-[2-{4-{1-Ethyl- 1-[4-(3-hydroxy- 4,4-dimethyl- pentyl)-3-methyl- phenyl]-propyl}-2- methyl-phenoxy)- acetylamino]-4- methyl-pentanoic acid | 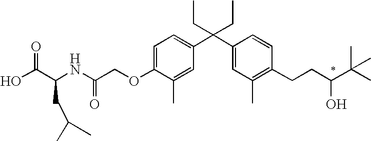 | 0.59 (t, 6H), 0.87 (s, 9H), 0.86-0.90 (m, 6H), 2.06 (q, 4H), 2.24 (s, 6H), 3.15 (d, | | | |
| # 1H), 4.40 (t, 1H), 6.73 (d, 1H), 6.90-7.03 (m, 5H) | 571 [M + NH₄]⁺ | 0.6 | 5 | | | |
| 218 | 2(R)-[2-(4-{1-Ethyl- 1-(4-(3-hydroxy- 4,4-dimethyl- pentyl)-3-methyl- phenyl]-propyl}-2- methyl-phenoxy)- acetylamino]-4- methyl-pentanoic acid | 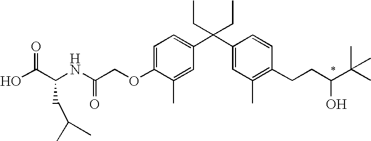 | 0.59 (t, 6H), 0.87 (s, 9H), 0.86-0.92 (m, 6H), 2.06 (s, 6H), 3.18 (d, 1H), 4.41 (t, 1H), 4.43-4.58 | | | |
| # (m, 2H), 6.73 (d, 1H), 6.89-7.03 (m, 5H) | 571 [M + NH₄]⁺ | 2.0 | 18 | | | |
| 219 | (S)-Cyclohexyl-(2- (4-1-ethyl-1-{4-(3- hydroxy-4,4- dimethyl-pentyl)-3- methyl-phenyl]- propyl]-2-methyl- phenoxy)- acetylamino]-acetic acid | 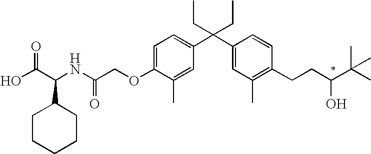 | 0.59 (t, 6H), 0.87 (s, 9H), 2.06 (q, 4H), 2.24 (s, 3H), 2.25 (s, 3H), 3.15 (d, 1H), 4.31 (d, 1H), | | | |
| # 4.52-4.59 (m, 2H), 6.74(d, 1H), 6.89-7.01 (m, 5H) | 597 [M + NH₄]⁺ | 1.5 | 13 | | | |
| 220 | (S)-3-Cyclohexyl-2- [2-(4-{1-ethyl-1-{4- (3-hydroxy-4,4- dimethyl-pentyl)-3- methyl-phenyl]- propyl}-2-methyl- phenoxy)- acetylamino]- propionic acid | 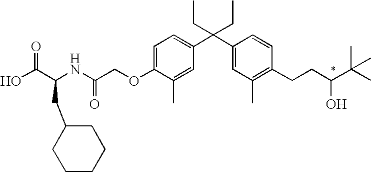 | 0.59 (t, 6H), 0.87 (s, 9H), 2.06 (q, 4H), 2.24 (s, 6H), 3.16 (d, 1H), 4.48-4.60 (m, 3H), 6.72 (d, 1H), | | | |
| # 6.92-7.03 (m, 5H) | 611 [M + NH₄]⁺ | 0.6 | 5 | | | |
| 221 | (S)-1-[2-(4-(1-Ethyl- 1-[4-(3-hydroxy- 4,4-dimethyl- pentyl)-3-methyl- phenyl]-propyl}-2- methyl-phenoxy)- acetyl]-pyrrolidine- 2-carboxylic acid | 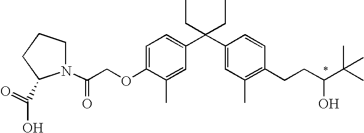 | 0.58 (t, 6H), 0.87 (s, 9H), 2.06 (q, 4H), 2.24 (s, 3H), 3.16 (d, 1H), | | | |
| # 4.48-4.60 (m, 3H), 6.72 (d, 1H), 6.92-7.03 (m, 5H) | 555 [M + NH₄]⁺ | 0.4 | 4 | | | |
| 222 | (S)-[2-(4-{1-Ethyl-1- [4-(3-hydroxy-4,4- dimethyl-pentyl)-3- methyl-phenyl}- propyl}-2-methyl- phenoxy)- acetylamino}- phenyl-acetic acid | 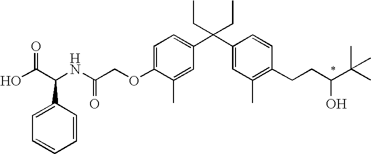 | 0.60 (t, 6H), 0.87 (s, 9H), 2.05 (q, 4H), 2.24 (s, 3H), 2.28 (s, 3H), 3.18 (d, 1H), 4.53-4.60 (m, 2H), | | | |
| # 5.35 (s, 1H), 6.72 (d, 1H), 6.90-7.04 (m, 5H), 7.26-7.38 (m, 5H) | 591 [M + NH₄]⁺ | 0.6 | 5 | | | |
| 223 | 2(S)-(2-(4-(1-Ethyl- 1-(4-(3-hydroxy- 4,4-dimethyl- pentyl)-3-methyl- phenyl]-propyl}-2- methyl-phenoxy)- acetylamino]-3- phenyl-propionic acid | 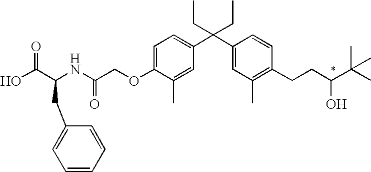 | 0.59 (t, 6H), 0.87 (s, 9H), 2.06-2.10 (m, 7H), 2.22 (s, 3H), 4.39-4.53 (m, 2H), 4.70 1H), 6.59 (d, 1H), | | | |
| # 6.90-7.17 (m, 10H) | 605 [M + NH₄]⁺ | 0.9 | 8 | | | |
| 224 | 2(R)-{2-(4-{1-Ethyl- 1-(4-(3-hydroxy- 4,4-dimethyl- pentyl)-3-methyl- phenyl]-propyl}-2- methyl-phenoxy)- acetylamino]-3- phenyl-propionic acid | 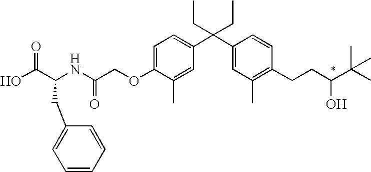 | 0.59 (t, 6H), 0.86 (s, 9H), 2.05-2.10 (m, 7H), 2.22 (s, 3H), 4.36-4.51 (m, 2H), 4.65 | | | |
| # (t, 1H), 6.60 (d, 1H), 6.90-7.16 (m, 10H) | 605 [M + NH₄]⁺ | 3.6 | 31 | | | |
| 225 | 2(S)-[2-(4-{1-Ethyl- 1-{4-(3-hydroxy- 4,4-dimethyl- pentyl)-3-methyl- phenyl]-propyl}-2- methyl-phenoxy)- acetylamino]-3-(4- hydroxy-phenyl)- propionic acid | 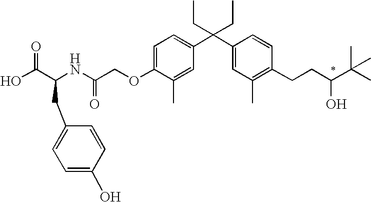 | 0.60 (t, 6H), 1.29 (s, 9H), 2.05-2.11 (m, 7H), 2.22 (s, 3H), 3.17 (d, 1H), 4.40-4.53 | | | |
| # (t, 1H), 6.60-7.03 (m, 10H) | 621 [M + NH₄]⁺ | 2.2 | 18 | | | |
| 226 | 2(R)-[2-(4-{1-Ethyl- 1-(4-(3-hydroxy- 4,4-dimethyl- pentyl)-3-methyl- phenyl]-propyl}-2- methyl-phenoxy)- acetylamino]-3-(4- hydroxy-phenyl)- propionic acid | 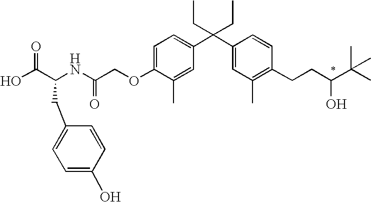 | 0.60 (t, 6H), 0.87 (s, 9H), 2.06-2.11 (m, 7H), 2.21 (s, | | | |
| # 3H), 3.15 (d, 1H), 4.37-4.52 (m, 2H), 4.62 (t, 1H), 6.59-7.03 (m, 10H) | 621 [M + NH₄]⁺ | 2.9 | 24 | | | |
| 227 | (2S,3R)-2-(2-(4-{1- Ethyl-1-[4-(3- hydroxy-4,4- dimethyl-pentyl)-3- methyl-phenyl]- propyl]-2-methyl- phenoxy)- acetylamino]-3- hydroxy-butyric acid | 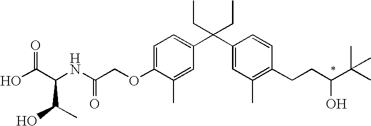 | 0.63 (t, 6H), 0.91 (s, 9H), 1.13 (d, 3H), 2.12 (q, 4H), 2.27 (s, 3H), 2.29 (s, 3H), | | | |
| # 3.19 (d, 1H), 4.27-4.34 (m, 2H), 4.59 (s, 2H), 6.78-7.07 (m, 6H) | 559 [M + NH₄]⁺ | 1.3 | 12 | | | |
| 228 | (2R,3S)-2-[2-C4-{1- Ethyl-1-[4-(3- hydroxy-4,4- dimethyl-pentyl)-3- methyl-phenyl]- propyl}-2-methyl- phenoxy)- acetylamino]-3- hydroxy-butyric acid | 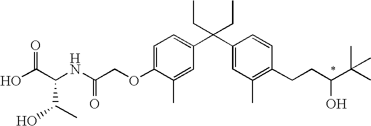 | 0.59 (t ,6H), 0.87 (s, 9H), 1.10 (d, 3H), 2.04 (q, 4H), | | | |
| # 2.24 (s, 3H), 2.25 (d, 3H), 3.15 (d, 1H), 4.28-4.59 (m, 4H), 6.78-7.03 (m, 6H) | 559 [M + NH₄]⁺ | 0.7 | 6 | | | |
| 229 | 2(S)-[2-(4-{1-Ethyl- 1-[4-(3-hydroxy- 4.4-dimethyl- pentyl)-3-methyl- phenyl}-propyl]-2- methyl-phenoxy}- acetylamino]-3- hydroxy-propionic acid | 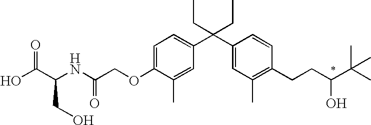 | 0.63 (t, 6H), 0.91 (s, 9H), 2.11 (s, 4H), 2.28 (s, 3H), 2.31 (s, 3H), 3.19 (d, 1H), | | | |
| # 3.94 (d, 2H), 4.37 (t, 1H), 4.55 (s, 3H), 6.82-7.07 (m, 6H) | 545 [M + NH₄]⁺ | 1.7 | 16 | | | |
| 230 | 2(R)-[2-(4-{1-Ethyl- 1-[4-(3-hydroxy- 4,4-dimethyl- pentyl)-3-methyl- phenyl]-propyl}-2- methyl-phenoxy)- acetylamino]-3- hydroxy-propionic acid | 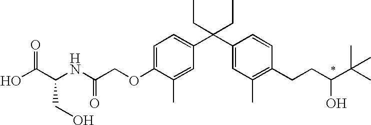 | 0.59 (t, 6H), 0.87 (s, 9H), 2.07 (q, 4H), 3.18 (d, 1H), 3.90-3.93 (m, | | | |
| # 2H), 4.37 (t, 1H), 4.50-4.55 (m, 2H), 6.79 (d, 1H), 6.90-7.03 (m, 5H) | 545 [M + NH₄]⁺ | 2.0 | 19 | | | |
| 231 | (2S,4R)-1-[2-(4-{1- Ethyl-1-[4-(3- hydroxy-4,4- dimethyl-pentyl)-3- methyl-phenyl]- propyl}-2-methyl- phenoxy)-acetyl]-4- hydroxy-pyrrolidine- 2-carboxylic acid | 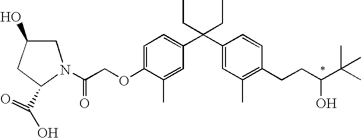 | 0.62 (t, 6H), 0.91 (s, 9H), 2.08 (q, 4H), 2.22 (s, 3H), 2.27 (s, 3H), | | | |
| # 3.19 (d, 1H), 3.69-3.72(m, 1H), 4.43-4.75 (m, 3H), 6.77-7.07 (m, 6H) | 571 [M + NH₄]⁺ | 1.6 | 14 | | | |
| 232 | (S)-5-Amino-2-[2- (4-(1-ethyl-1-[4-(3- hydroxy-4,4- dimethyl-pentyl)-3- methyl-phenyl]- propyl]-2-methyl- phenoxy)- acetylamino]- pentanoic acid | 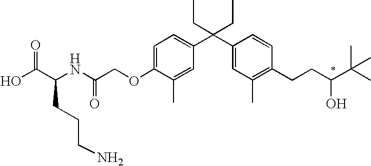 | 0.63 (t, 6H), 0.91 (s, 9H), 2.10 (q, 4H), 2.28 (s, 3H), 2.30 (s, 3H), 2.99 (t, 2H), 3.15 (d, 1H), 4.32 (t, 1H), | | | |
| # 4.54 (s, 2H), 6.80 (d, 1H), 6.94-7.07 (m, 5H) | 572 [M + NH₄]⁺ | 2.7 | 24 | | | |
| 233 | (R)-5-Amino-2-[2- (4-[1-ethyl-1-[4-(3- hydroxy-4,4- dimethyl-pentyl)-3- methyl-phenyl]- propyl}-2-methyl- phenoxy)- acetylamino]- pentanoic acid | 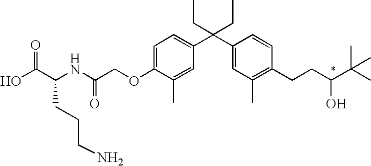 | 0.63 (t, 6H), 0.91 (s, 9H), 2.11 (q, 4H), 2.28 (s, 3H), 2.29 (s, 3H), 2.99 (t, 2H), | | | |
| # 3.19 (d, 1H), 4.33 (t, 1H), 4.54 (s, 2H), 6.79 (d, 1H), 6.93-7.08 (m, 5H) | 572 [M + NH₄]⁺ | 3.5 | 32 | | | |
| 234 | (S)-6-Amino-2-[2- (4-(1-ethyl-1-[4-(3- hydroxy-4,4- dimethyl-pentyl)-3- methyl-phenyl]- propyl)-2-methyl- phenoxy)- acetylamino]- hexanoic acid | 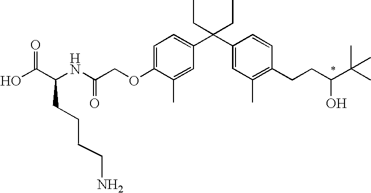 | 0.59 (t, 6H), 0.87 (s, 9H), 2.06 (q, 4H), 2.25 (s, 3H), 2.88 (t, 2H), 3.15 (d, 1H), 4.31 (t, 1H), | | | |
| # 4.45 (s, 2H), 6.75 (d, 1H), 6.92-7.03 (m, 5H) | 586 [M + NH₄]⁺ | 2.3 | 20 | | | |
| 235 | (R)-6-Amino-2-[2- (4-(1-ethyl-1-[4-(3- hydroxy-4,4- dimethyl-pentyl)-3- methyl-phenyl]- propyl]-2-methyl- phenoxy)- acetylamino]- hexanoic acid | 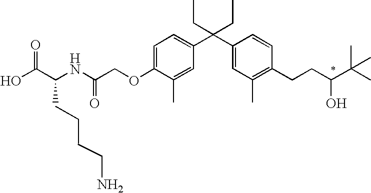 | 0.59 (t, 6H), 0.87 (s, 9H), 2.06 (q, 4H), 2.24 (s, 3H), 2.26 (s, 3H), 2.88 (t, 2H), 3.15 (d, 1H), 4.31 (t, 1H), | | | |
| # 4.50 (s, 2H), 6.76 (d, 1H), 6.90-7.03 (m, 5H) | 586 [M + NH₄]⁺ | 1.3 | 11 | | | |
| 236 | 2(S)-[2-(4-(1-Ethyl- 1-[4-(3-hydroxy- 4,4-dimethyl- pentyl)-3-methyl- phenyl]-propyl]-2- methyl-phenoxy)- acetylamino]- succinic acid | 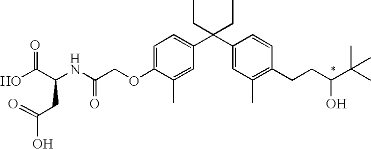 | 0.59 (t, 6H), 0.87 (s, 9H), 2.06 (q, 4H), 2.24 (s, 6H), 3.15 (d, 1H), 4.47-4.59 (m, 3H), 6.75-7.03 (m, 6H) | | | |
| # 573 [M + NH₄]⁺ | 1.9 | 17 | | | | |
| 237 | 2(R)-[2-(4-(1-Ethyl- 1-[4-(3-hydroxy- 4,4-dimethyl- pentyl)-3-methyl- phenyl]-propyl]-2- methyl-phenoxy)- acetylamino]- succinic acid | 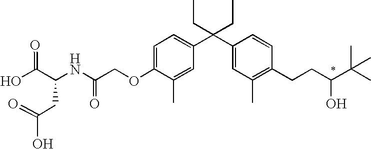 | 0.59 (t, 6H), 0.87 (s, 9H), 2.04 (q, 4H), 2.24 (s, 6H), 3.15 (d, 1H), 4.46-4.60 (m, | | | |
| # 3H), 6.77 (d, 1H), 6.88-7.03 (m, 5H) | 573 [M + NH₄]⁺ | 0.6 | 5 | | | |
| 238 | 2(S)-[2-(4-(1-Ethyl- 1-[4-(3-hydroxy- 4,4-dimethyl- pentyl)-3-methyl- phenyl]-propyl]-2- methyl-phenoxy)- acetylamino]- pentanedoic acid | 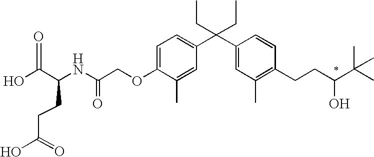 | 0.58 (t, 6H), 0.87 (s, 9H), 2.06 (q, 4H), 2.24 (s, 3H), 2.26 (s, 3H), 3.15 (d, 1H), | | | |
| # 4.37 (t, 1H), 4.51 (s, 2H), 6.76 (d, 1H), 6.91-7.03 (m, 5H) | 587 [M + NH₄)⁺ | 3.0 | 26 | | | |
| 239 | 2(R)-[2-(4-(1-Ethyl- 1-[4-(3-hydroxy- 4,4-dimethyl- pentyl)-3-methyl- phenyl]-propyl]-2- methyl-phenoxy)- acetylamino]- pentanedioc acid | 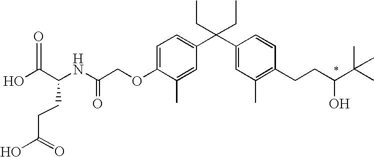 | 0.59 (t, 6H), 0.87 (s, 9H), 2.06 (q, 4H), 2.24 (s, 3H), 2.25 (s, 3H), 3.15 (d, 1H), | | | |
| # 4.32-4.38 (m, 1H), 4.50 (s, 2H), 6.76 (d, 1H), 6.88-7.03 (m, 5H) | 587 [M + NH₄]⁺ | 2.3 | 20 | | | |
| 240 | 2(S)-[2-(4-(1-Ethyl- 1-[4-((S)-3-hydroxy- 4,4-dimethyl- pentyl)-3-methyl- phenyl}-propyl]-2- methyl-phenoxy)- acetylamino]- succinamic acid | 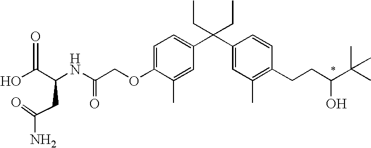 | 0.60 (t, 6H), 0.91 (s, 9H), 2.09 (q, 4H), 2.28 (s, 3H), 2.29 (s, 3H), 2.79 (d, 2H), | | | |
| # 3.19 (d, 1H), 4.53-4.63 (m, 3H), 6.78-7.05 (m, 6H) | 572 [M + NH₄]⁺ | 3.9 | 35 | | | |
| 241 | 2(R)-[2-(4-(1-Ethyl- 1-[4-((S)-3-hydroxy- 4,4-dimethyl- pentyl)-3-methyl- phenyl]-propyl]-2- methyl-phenoxy)- acetylamino]- succinamic acid | 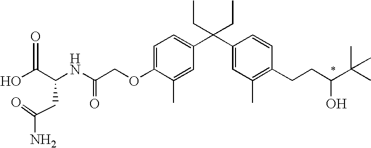 | 0.63 (t, 6H), 0.91 (s, 9H), 2.10 (q, 4H), 2.28 (s, 3H), 2.29 (s, 3H), 3.20 (d, 1H), 4.53 (d, 2H), 4.64 (t, 1H), | | | |
| # 6.79-7.04 (m, 6H) | 572 [M + NH₄]⁺ | 1.1 | 10 | | | |
| 242 | (S)-4-Carbamoyl-2- [2-(4-(1-ethyl-1-[4- ((S)-3-hydroxy-4,4- dimethyl-pentyl)-3- methyl-phenyl]- propyl}-2-methyl- phenoxy) acetylamino]- butyric acid | 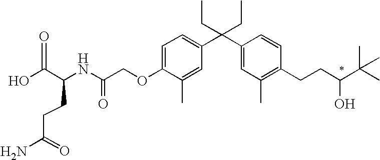 | 0.60 (t, 6H), 0.91 (s, 9H), 2.07 (q, 4H), 2.26 (s, 3H), 2.28 (s, 3H), | | | |
| # 3.20 (d, 1H), 4.36-4.38 (m, 1H), 4.55 (s, 2H), 6.78-7.07 (m, 6H) | 586 [M + NH₄]⁺ | 2.8 | 25 | | | |
| 243 | (R)-4-Carbamoyl-2- [2-(4-(1-ethyl-1-[4- ((S)-3-hydroxy-4,4- dimethyl-pentyl)-3- methyl-phenyl]- propyl}-2-methyl- phenoxy)- acetylamino]- butyric acid | 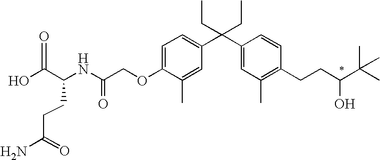 | 0.58 (t, 6H), 0.87 (s, 9H), 2.07 (q, 4H), 2.24 (s, 3H), 2.26 (s, 3H), 3.16 (d, 1H), 4.35-4.39 (m, | | | |
| # 1H), 4.51 (s, 2H), 6.78-7.05 (m, 6H) | 586 [M + NH₄]⁺ | 3.2 | 28 | | | |

| | | amino acid | |
|---|---|---|---|
| | | loading on | |
| | | resin | reacted resin |
| Example # | Fmoc-amino acid-Wang resin | (mmol/g) | (mg) |
| 204 | Fmoc-beta-Ala-Wang resin | 0.52 | 38.5 |
| 205 | Fmoc-4-Abu-Wang resin | 0.41 | 48.8 |
| 206 | Fmoc-L-Ala-Wang resin | 0.78 | 25.6 |
| 207 | Fmoc-D-Ala-Wang resin | 0.54 | 37.0 |
| 208 | Fmoc-L-Abu(2)-Wang resin | 0.40 | 50.0 |
| 209 | Fomc-L-Nva-Wang resin | 0.67 | 29.9 |
| 210 | Fmoc-D-Nva-Wang resin | 0.76 | 26.3 |
| 211 | Fmoc-L-Nle-Wang resin | 0.73 | 27.4 |
| 212 | Fmoc-D-Nle-Wang resin | 0.65 | 30.8 |
| 213 | Fmoc-L-Val-Wang resin | 0.80 | 25.0 |
| 214 | Fmoc-D-Val-Wang resin | 0.65 | 30.8 |
| 215 | Fmoc-L-Ile-Wang resin | 0.86 | 23.3 |
| 216 | Fmoc-D-Ile-Wang resin | 0.59 | 33.9 |
| 217 | Fmoc-L-Leu-Wang resin | 0.86 | 23.3 |
| 218 | Fmoc-D-Leu-Wang resin | 0.56 | 35.7 |
| 219 | Fmoc-L-Chg-Wang resin | 0.71 | 28.2 |
| 220 | Fmoc-L-Cha-Wang resin | 0.72 | 27.8 |
| 221 | Fmoc-L-Pro-Wang resin | 0.72 | 27.8 |
| 222 | Fmoc-L-Phg-Wang resin | 0.75 | 26.7 |
| 223 | Fmoc-L-Phe-Wang resin | 0.78 | 25.6 |
| 224 | Fmoc-D-Phe-Wang resin | 0.52 | 38.5 |
| 225 | Fmoc-L-Tyr(tBu)-Wang resin | 0.79 | 25.3 |
| 226 | Fmoc-D-Tyr(tBu)-Wang resin | 0.66 | 30.3 |
| 227 | Fmoc-L-Thr(tBu)-Wang resin | 0.71 | 28.2 |
| 228 | Fmoc-D-Thr(tBu)-Wang resin | 0.76 | 26.3 |
| 229 | Fmoc-L-Ser(tBu)-Wang resin | 0.81 | 24.7 |
| 230 | Fmoc-D-Ser(tBu)-Wang resin | 0.74 | 27.0 |
| 231 | Fmoc-L-Hyp(tBu)-Wang resin | 0.77 | 26.0 |
| 232 | Fmoc-L-Orn(Boc)-Wang resin | 0.48 | 41.7 |
| 233 | Fmoc-D-Orn(Boc)-Wang resin | 0.30 | 66.7 |
| 234 | Fmoc-L-Lys(Boc)-Wang resin | 0.55 | 36.4 |
| 235 | Fmoc-D-Lys(Boc)-Wang resin | 0.69 | 29.0 |
| 236 | Fmoc-L-Asp(OtBu)-Wang resin | 0.59 | 33.9 |
| 237 | Fmoc-D-Asp(OtBu)-Wang resin | 0.61 | 32.8 |
| 238 | Fmoc-L-Glu(OtBu)-Wang resin | 0.68 | 29.4 |
| 239 | Fmoc-D-Glu(OtBu)-Wang resin | 0.71 | 28.2 |
| 240 | Fmoc-L-Asn(Trt)-Wang resin | 0.54 | 37.0 |
| 241 | Fmoc-D-Asn(Trt)-Wang resin | 0.50 | 40.0 |
| 242 | Fmoc-L-Gln(Trt)-Wang resin | 0.51 | 39.2 |
| 243 | Fmoc-D-Gln(Trt)-Wang resin | 0.73 | 27.4 |

### Example 244

### Preparation of 3-(4-{1-Ethyl-1-[4-(3-ethyl-3-hydroxy-pentyl)-3-methyl-phenyl]-propyl}-2-methyl-phenoxy)-propane-1,2-diol

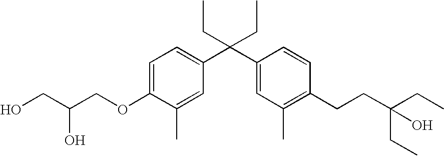

### (1) Preparation of 4-{1-[4-(2,2-Dimethyl-[1,3]dioxolan-4-ylmethoxy)-3-methyl-phenyl]-1-ethyl-propyl}-2-methyl-phenol

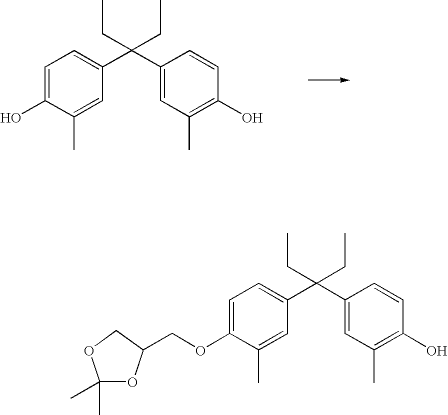

To a solution of 3,3-bis-(4-hydroxy-3-methyl-phenyl)-pentane (2.91 g, 10.2 mmol) in DMSO (26 ml) were added t-BuOK (1.1 g, 9.80 mmol) and 2,2-dimethyl-1,3-dioxolan-4-yl-methyl p-toluensulfonate (2.3 g, 8.03 mmol) and the mixture was stirred at 60 degrees C. for 8 h. The reaction mixture was poured into sat. NH₄Cl aq. and the products were extracted with AcOEt. The extracts were washed with water and brine, dried over Na₂SO₄, filtered and concentrated under reduced pressure. The obtained residue was chromatographed on silica gel with CH₂Cl₂:AcOEt (50/1 to 2/1) to give the title compound (1.37 g, 43%) as colorless oil.

¹H NMR (CDCl₃, 400 MHz): 0.61 (6H, t, J=7.2 Hz), 1.40 (3H, s), 1.46 (3H, s), 2.00 (4H, q, J=7.5 Hz), 2.15 (3H, s), 2.19 (3H, s), 3.89-4.18 (4H, m), 4.60 (1H, s), 6.67 (2H, m), 6.83-6.96 (4H, m).

### (2) Preparation of Trifluoro-methanesulfonic acid 4-{1-[4-(2,2-dimethyl-[1,3]dioxolan-4-ylmethoxy)-3-methyl-phenyl]-1-ethyl-propyl}-2-methyl-phenyl ester

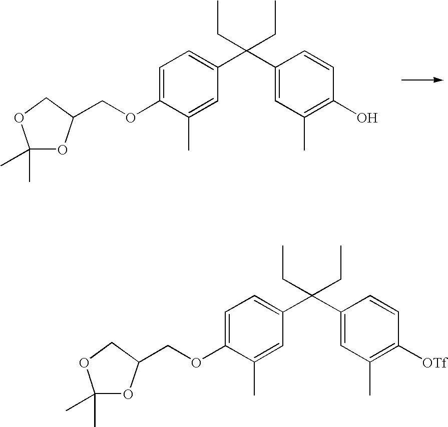

To a solution of 4-{1-[4-(2,2-Dimethyl-[1,3]dioxolan-4-ylmethoxy)-3-methyl-phenyl]-1-ethyl-propyl}-2-methyl-phenol (compound prepared in Example 244-(1)) (1.57 g, 3.939 mmol) in CH₂Cl₂ (40 ml) under N₂ atmosphere were added

Tf₂O (0.969 ml, 5.909 mmol) and pyridine (0.954 ml, 11.82 mmol) and the mixture was stirred for 30 min. The reaction mixture was poured into sat. NaHCO₃ aq. and the products were extracted with CH₂Cl₂. The extracts were dried over MgSO₄, filtered and concentrated under reduced pressure. The obtained residue was chromatographed on silica gel with EtOAc-n-Hexane (1:20) to give the title compound (1.5 g, 50%) as colorless oil.

¹H-NMR (CDCl₃): 0.59 (6H, t, J=7.25 Hz), 1.40 (3H, s), 1.46 (3H, s), 1.97-2.11 (4H, m), 2.16 (3H, s), 2.31 (3H, s), 3.90-4.20 (4H, m), 4.40-4.52 (1H, m), 6.70 (1H, d, J=8.57 Hz), 6.81-6.95 (2H, m), 7.00-7.15 (3H, m).

### (3) Preparation of 3-(4-{1-[4-(2,2-Dimethyl-[1,3]dioxolan-4-ylmethoxy)-3-methyl-phenyl]-1-ethyl-propyl}-2-methyl-phenyl)-propionic acid ethyl ester

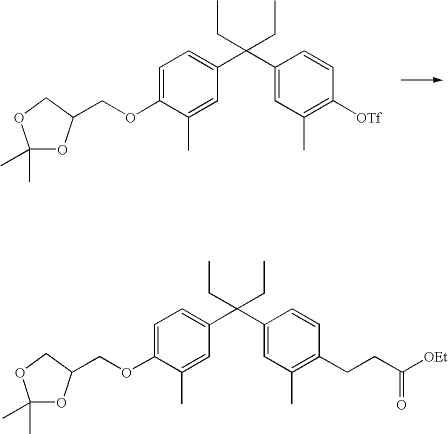

To a solution of Trifluoro-methanesulfonic acid 4-{1-[4-(2,2-dimethyl-[1,3]dioxolan-4-ylmethoxy)-3-methyl-phenyl]-1-ethyl-propyl}-2-methyl-phenyl ester (compound prepared in Example. 244-(2)) (310 mg, 0.584 mmol) in DMF (1.0 ml) was added Pd(PPh₃)₄ (67 mg) and the mixture was stirred for 5 min. To the reaction mixture, 3-Ethoxy-3-oxopropylzinc bromide 0.5 M THF solution (2.9 ml, 1.45 mmol) and HMPA (1.45 ml, 8.30 mmol) were added and the mixture was stirred at 60 degrees C. for 3.5 h. The reaction mixture was poured into water and the products were extracted with AcOEt. The extracts were dried over Na₂SO₄, filtered and concentrated under reduced pressure. The obtained residue was chromatographed on silica gel with EtOAc-n-Hexane (1:5) to give the title compound (175 mg, 62%) as colorless oil.

¹H NMR (CDCl₃); 0.58 (6H, t, J=7.4 Hz), 1.24 (3H, t, J=7.1 Hz), 1.40 (3H, s), 1.46 (3H, s), 2.01 (4H, q, J=7.4 Hz), 2.15 (3H, s), 2.25 (3H, s), 2.56 (2H, t J=7.4 Hz), 2.89 (2H, t, J=8.5 Hz), 3.93-4.11 (m, 6H), 4.46 (m, 1H), 6.68 (1H, d, J=8.5 Hz), 6.94 (5H, m).

### (4) Preparation of 1-(4-{1-[4-(2,2-Dimethyl-[1,3]dioxolan-4-ylmethoxy)-3-methyl-phenyl]-1-ethyl-propyl}-2-methyl-phenyl)-3-ethyl-pentan-3-ol

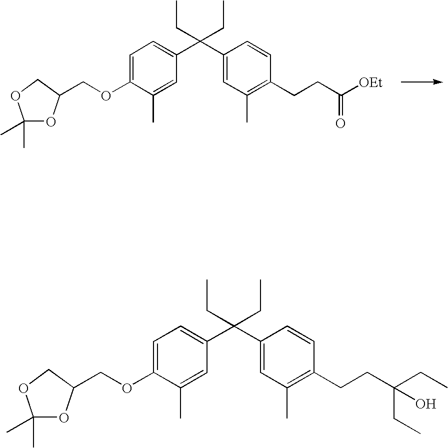

To a solution of 3-(4-{1-[4-(2,2-Dimethyl-[1,3]dioxolan-4-ylmethoxy)-3-methyl-phenyl]-1-ethyl-propyl}-2-methyl-phenyl)-propionic acid ethyl ester (compound prepared in Example 244-(3)) (37.5 mg, 0.078 mmol) in THF (2 ml) at −78 degrees C. under N₂ atmosphere was added EtLi (ca.0.4 mol/l, 0.585 ml, 0.234 mmol) and the mixture was stirred for 1 h. The reaction mixture was poured into sat. NH₄Cl aq. and the products were extracted with AcOEt. The extracts were washed with water and brine, dried over MgSO₄, filtered and concentrated under reduced pressure. The obtained residue was purified by preparative TLC (Hex:AcOEt 2:1) to give the title compound (30.4 mg, 79%) as colorless oil.

¹H-NMR (CDCl₃): 0.59 (6H, t, J=7.08 Hz), 0.91 (6H, t, J=7.42 Hz), 1.40 (3H, s), 1.46 (3H, s), 1.48-1.70 (6H, m), 1.95-2.05 (4H, m), 2.16 (3H, s), 2.25 (3H, s), 2.50-2.61 (2H, m), 3.89-4.20 (4H, m), 4.40-4.50 (1H, m), 6.68 (1H, d, J=8.24 Hz), 6.85-7.03 (5H, m).

### (5) Preparation of 3-(4-{1-Ethyl-1-[4-(3-ethyl-3-hydroxy-pentyl)-3-methyl-phenyl]-propyl}-2-methyl-phenoxy)-propane-1,2-diol

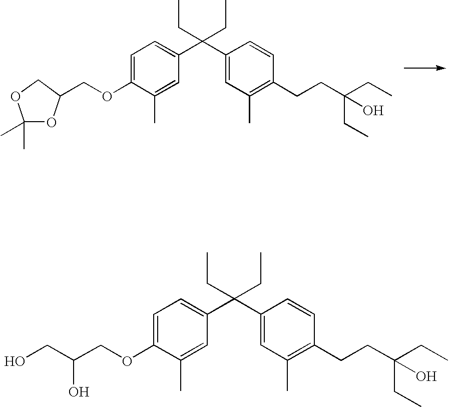

To a solution of 1-(4-{1-[4-(2,2-Dimethyl-[1,3]dioxolan-4-ylmethoxy)-3-methyl-phenyl]-1-ethyl-propyl}-2-methyl-phenyl)-3-ethyl-pentan-3-ol (compound prepared in Example 244-(4)) (30.4 mg, 0.061 mmol) in THF-H₂O (10:1, 3.3 ml) was added CSA (42.7 mg, 0.184 mmol) and the mixture was stirred at 50 degrees C. for 5 h. The reaction mixture was poured into sat. NaHCO₃ aq. and the products were extracted with CH₂Cl₂. The extracts were dried over MgSO₄, filtered and concentrated under reduced pressure. The obtained residue was purified by preparative TLC (AcOEt) to give the title compound (19.4 mg, 70%) as colorless oil.

¹H-NMR (CDCl₃): 0.59 (6H, t, J=7.09 Hz), 0.91 (6H, t, J=7.42 Hz), 1.48-1.70 (6H, m), 1.95-2.10 (4H, m), 2.17 (3H, s), 2.25 (3H, s), 2.50-2.62 (2H, m), 3.72-3.90 (2H, m), 3.98-4.15 (3H, m), 6.69 (1H, d, J=8.25 Hz), 6.85-7.03 (5H, m); MS (ESI+): 474 ([M+NH₄]⁺).

### Example 246

### Preparation of (S)-5-(4-{1-Ethyl-1-[4-((E)-3-ethyl-3-hydroxy-1-methyl-pent-1-enyl)-3-methyl-phenyl]-propyl}-2-methyl-phenoxy)-4-hydroxy-pentanoic acid

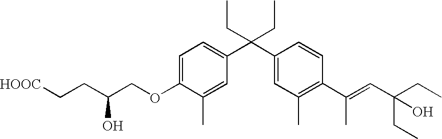

### (1) Preparation of (E)-3-{4-[1-Ethyl-1-(4-hydroxy-3-methyl-phenyl)-propyl]-2-methyl-phenyl}-but-2-enoic acid ethyl ester

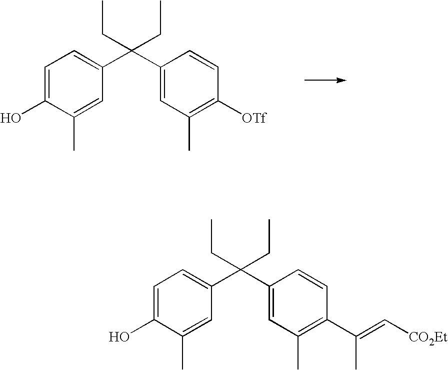

To a solution of Trifluoro-methanesulfonic acid4-[1-ethyl-1-(4-hydroxy-3-methyl-phenyl)-propyl]-2-methyl-phenyl ester (compound prepared in Example 1-(I)) (52.5 mg, 0.105 mmol) in DMF(0.35 ml) were added Crotonic acid ethyl ester (0.052 ml, 0.419 mmol), NaHCO₃ (37 mg, 0.440 mmol), dppp(4.3 mg, 0.0104 mmol), LiBr(6.3 mg, 0.0725 mmol) and PdCl₂ (PPh₃) ₂ (7.4 mg, 0.0105 mmol) and the mixture was stirred at 140 degrees C. for 12 h. The reaction mixture was cooled to room temperature, poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:4) to give the title compound (13.7 mg, 28%) as colorless oil.

¹H-NMR (CDCl₃): 0.58 (6H, t, J=7.09 Hz), 1.28 (3H, t, J=7.25 Hz), 2.05 (4H, q, J=7.26 Hz), 2.20 (3H, s), 2.22 (3H, s), 2.43 (3H, s), 4.19 (2H, q, J=7.25 Hz), 4.65 (1H, s), 5.76 (1H, s), 6.66 (1H, d, J=8.24 Hz), 6.95 (5H, m).

### (2) Preparation of 4-{1-Ethyl-1-[4-((E)-3-ethyl-3-hydroxy-1-methyl-pent-1-enyl)-3-methyl-phenyl]-propyl}-2-methyl-phenol

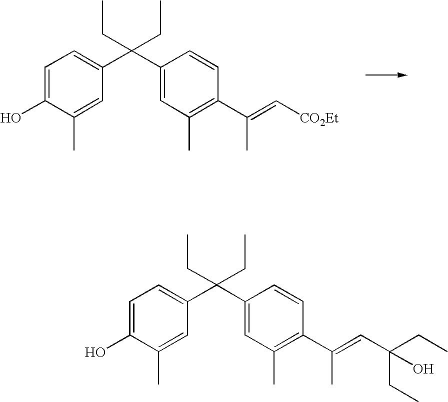

To a solution of (E)-3-{4-[1-Ethyl-1-(4-hydroxy-3-methyl-phenyl)-propyl]-2-methyl-phenyl)-but-2-enoic acid ethyl ester (compound prepared in Example 246-(1))(64 mg, 0.168 mmol) in THF(2 ml) was added EtMgBr(3M in Et₂O, 0.28 ml, 0.840 mmol) at 0 degrees C. under nitrogen atmosphere and the reaction mixture was stirred for 2.5 h at room temperature. The reaction mixture was poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:2) to give the title compound (30 mg, 45%) as colorless oil.

¹H-NMR (CDCl₃): 0.61 (6H, t, J=7.25 Hz), 0.97 (6H, t, J=7.41 Hz), 1.66 (4H, q, J=7.42 Hz), 2.03 (4H, q, J=7.42 Hz), 2.11 (3H, d, J=0.99 Hz), 2.21 (3H, s), 2.23 (3H, s), 4.65 (1H, brs), 5.18 (1H, d, J=1.16 Hz), 6.65 (1H, d, J=8.41 Hz), 6.85-6.92 (5H, m).

### (3) Preparation of (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one

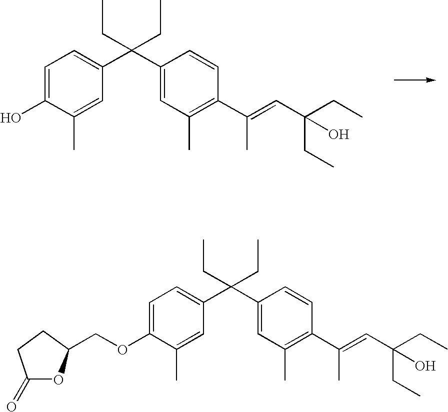

To a solution of 4-{1-Ethyl-1-[4-((E)-3-ethyl-3-hydroxy-1-methyl-pent-1-enyl)-3-methyl-phenyl]-propyl}-2-methyl-phenol (compound prepared in Example 246-(2)) (15 mg, 0.038 mmol) in DMF(1 ml) were added K₂CO₃(10.5 mg, 0.076 mmol) and (S)-Dihydro-5-(toluenesulfonylmethyl)-2-(3H)furanone (20.5 mg, 0.076 mmol) and the mixture was stirred at 100 degrees C. for 7 h. The reaction mixture was poured into sat. NaHCO₃ aq. and the products were extracted with AcOEt. The-extracts were washed with water and brine, dried over MgSO₄, filtered and concentrated under reduced pressure. The obtained residue was chromatographed on silica gel with EtOAc-n-Hexane (1:2) to give the title compound (15.2 mg, 81%) as colorless oil.

¹H-NMR (CDCl₃): 0.60 (6H, t, J=7.25 Hz), 0.97 (6H, t, J=7.26 Hz), 1.66 (4H, q, J=7.26 Hz), 1.98-2.08 (4H, m), 2.11 (3H, d, J=0.82 Hz), 2.16 (3H, s), 2.23 (3H, s), 2.25-2.85 (4H, m), 4.05-4.20 (2H, m), 4.82-4.92 (1H, m), 5.18 (1H, s), 6.67 (1H, d, J=8.24 Hz), 6.85-7.00 (5H, m).

### (4) Preparation of (S)-5-(4-{1-Ethyl-1-[4-((E)-3-ethyl-3-hydroxy-1-methyl-pent-1-enyl)-3-methyl-phenyl]-propyl}-2-methyl-phenoxy)-4-hydroxy-pentanoic acid

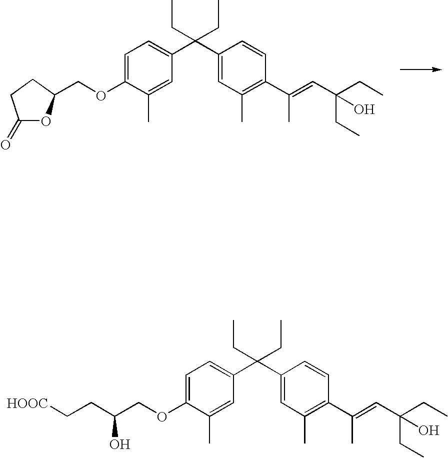

To a solution of (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one (compound prepared in Example 246-(3)) (15.8 mg, 0.038 mmol) in MeOH (0.360 ml) was added 1 N KOH(0.180 ml) and the mixture was stirred at room temperature for 1 h. The reaction mixture was concentrated under reduced pressure and the obtained residue was purified by preparative TLC (CHCl₃:MeOH=8:3, saturated with H₂O) to give the title compound (15.2 mg, 81%) as colorless oil.

¹H-NMR (CDCl₃): 0.61 (6H, t, J=7.09 Hz), 0.97 (6H, t, J=7.42 Hz), 1.66 (4H, q, J=7.25 Hz), 1.98-2.08 (6H, m), 2.11 (3H, s), 2.19 (3H, s), 2.23 (3H, s), 2.62 (2H, t, J=6.92 Hz), 3.85 (1H, t, J=9.07 Hz), 3.98 (1H, d, J=9.07 Hz), 4.10 (1H, m), 5.18 (1H, s), 6.68 (1H, d, J=8.07 Hz), 6.94 (5H, m); MS (ESI+) :528 ([M+NH₄]⁺).

### Example 247

### Preparation of (S)-5-(4-{1-[4-((E)-1,3-Diethyl-3-hydroxy-pent-1-enyl)-3-methyl-phenyl]-1-ethyl-propyl)-2-methyl-phenoxy)-4-hydroxy-pentanoic acid

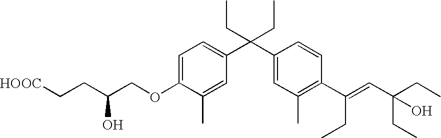

### (1) Preparation of 2,2-Dimethyl-propionic acid 4-[1-ethyl-1-(3-methyl-4-trifluoromethanesulfonyloxy-phenyl)-propyl]-2-methyl-phenyl ester

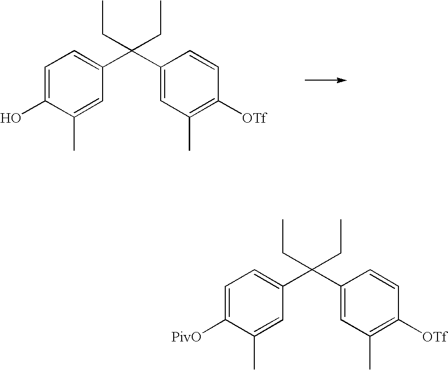

To a solution of Trifluoro-methanesulfonic acid 4-[1-ethyl-1-(4-hydroxy-3-methyl-phenyl)-propyl]-2-methyl-phenyl ester(compound prepared in Example 1-(1)) (1.75 g, 4.20 mmol) in CH₂Cl₂ (22 ml) were added Et₃N (0.643 ml, 4.64 mmol) and Pivaloyl chloride(0.566 ml, 4.60 mmol) and the mixture was stirred at 0 degrees C. for 1 h and at room temperature for 1 h. The reaction mixture was poured into water and the products were extracted with CH₂Cl₂. The extracts were dried over MgSO₄, filtered and concentrated under reduced pressure. The obtained residue was chromatographed on silica gel with EtOAc-n-Hexane (1:4) to give the title compound (1.66 g, 79%).

¹H-NMR (CDCl₃): 0.60 (6H, t, J=7.14 Hz), 1.37 (9H, s), 2.05 (4H, q, J=7.41 Hz), 2.12 (3H, s), 2.31 (3H, s), 6.87-7.13 (6H, m).

### (2) Preparation of (E)-3-(4-{1-[4-(2,2-Dimethyl-propionyloxy)-3-methyl-phenyl]-1-ethyl-propyl}-2-methyl-phenyl)-pent-2-enoic acid methyl ester

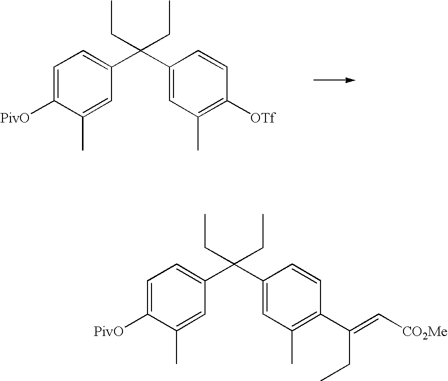

2,2-Dimethyl-propionic acid 4-[1-ethyl-1-(3-methyl-4-trifluoromethanesulfonyloxy-phenyl)-propyl]-2-methyl-phenyl ester(compound prepared in Example 247-(1)) (945 mg, 1.89 mmol) in DMF(6.3 ml) were added Methyl trans-2-pentanoate (0.929 ml, 7.33 mmol), NaHCO₃ (662 mg, 7.88 mmol), dppp(78 mg, 0.188 mmol), LiBr(117 mg, 1.35 mmol) and PdCl₂ (PPh₃)₂ (139 mg, 0.198 mmol) and the mixture was stirred at 150 degrees C. for 12 h. The reaction mixture was cooled to room temperature, poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:4) to give the title compound (253 mg, 29%) as colorless oil.

¹H-NMR (CDCl₃): 0.61 (6H, t, J=7.25 Hz), 1.301 (6H, t, J=7.09 Hz), 1.37 (9H, s), 2.06 (4H, q, J=7.25 Hz), 2.13 (3H, s), 2.23 (3H, s), 2.43 (3H, s), 4.20 (4H, q, J=7.25 Hz), 5.76 (1H, s), 6.87 (1H, d, J=8.41 Hz), 6.94-7.00 (5H, m).

### (3) Preparation of 2,2-Dimethyl-propionic acid 4-{1-[4-((E)-1,3-diethyl-3-hydroxy-pent-1-enyl)-3-methyl-phenyl]-1-ethyl-propyl}-2-methyl-phenyl ester

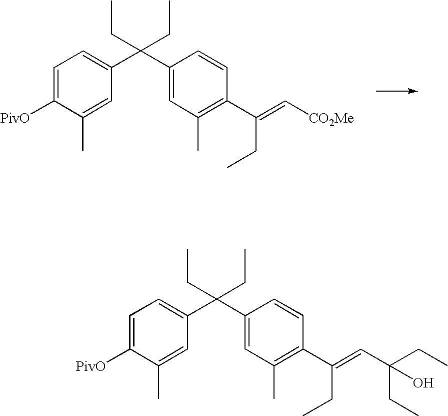

To a solution of (E)-3-(4-{1-[4-(2,2-Dimethyl-propionyloxy)-3-methyl-phenyl]-1-ethyl-propyl}-2-methyl-phenyl)-pent-2-enoic acid methyl ester (compound prepared in Example 247-(2))(128.1 mg, 0.276 mmol) in THF(3 ml) was added EtMgBr (3M in Et₂O, 0.552 ml, 1.656 mmol) at 0 degrees C. and the mixture was stirred at room temperature for 1 h. The reaction mixture was poured into sat. NH₄Cl aq. and the products were extracted with CH₂Cl₂. The extracts were dried over MgSO₄, filtered and concentrated under reduced pressure to give the title compound (124 mg, 91%) as colorless oil.

¹H-NMR (CDCl₃): 0.61 (6H, t, J=7.26 Hz), 0.85 (3H, t, J=7.42 Hz), 0.97 (6H, t, J=7.42 Hz), 1.36 (9H, s), 1.65 (4H, q, J=7.42 Hz), 2.06 (4H, q, J=7.25 Hz), 2.12 (3H, s), 2.22 (3H, s), 2.64 (2H, q, J=7.42 Hz), 5.11 (1H, s), 6.84-7.03 (6H, m).

### (4) Preparation of 4-{1-[4-((E)-1,3-Diethyl-3-hydroxy-pent-1-enyl)-3-methyl-phenyl]-1-ethyl-propyl}-2-methyl-phenol

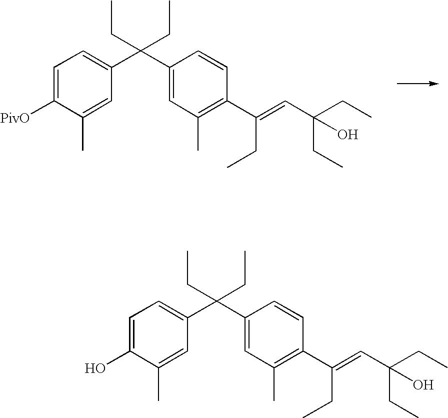

To a solution of 2,2-Dimethyl-propionic acid 4-{1-[4-((E)-1,3-diethyl-3-hydroxy-pent-1-enyl)-3-methyl-phenyl]-1-ethyl-propyl}-2-methyl-phenyl ester (compound prepared in Example 247-(3))(124 mg, 0.252 mmol) in MeOH (3 ml) was added 1N—KOH(0.503 ml, 0.503 mmol) and the mixture was stirred at room temperature for 1.5 h and at 60 degrees C. for 0.5 h. The reaction mixture was poured into sat. NH₄Cl aq. and the products were extracted with CH₂Cl₂. The extracts were dried over MgSO₄, filtered and concentrated under reduced pressure. The obtained residue was purified by preparative TLC (Hex:AcOEt 2:1) to give the title compound (99.5 mg, 97%) as colorless oil.

¹H-NMR (CDCl₃): 0.60 (6H, t, J=7.25 Hz), 0.85 (3H, t, J=7.42 Hz), 0.97 (6H, t, J=7.26 Hz), 1.66 (4H, q, J=7.25 Hz), 2.03 (4H, q, J=7.26 Hz), 2.20 (3H, s), 2.22 (3H, s), 2.64 (2H, q, J=7.42 Hz), 4.85 (1H, brs), 5.11 (1H, s), 6.65 (1H, d, J=8.24 Hz), 6.85-6.98 (5H, m).

### (5) Preparation of (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one

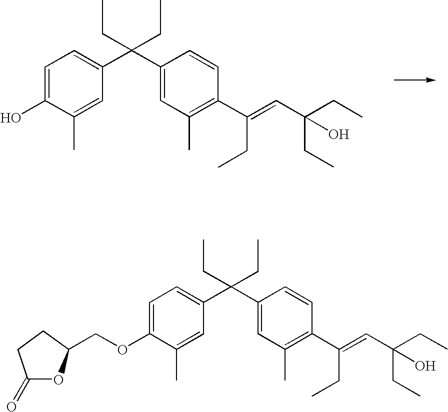

To a solution of 4-{1-[4-((E)-1,3-Diethyl-3-hydroxy-pent-1-enyl)-3-methyl-phenyl]-1-ethyl-propyl}-2-methyl-phenol (compound prepared in Example 247-(4)) (49 mg, 0.120 mmol) in DMF(1 ml) were added K₂CO₃(33.2 mg, 0.240 mmol) and (S)-Dihydro-5-(toluenesulfonylmethyl)-2-(3H)furanone (64.9 mg, 0.240 mmol) and the mixture was stirred at 100 degrees C. overnight. The reaction mixture was poured into sat. NaHCO₃ aq. and the products were extracted with AcOEt. The extracts were washed with water and brine, dried over MgSO₄, filtered and concentrated under reduced pressure. The obtained residue was purified by preparative TLC (Hex:AcOEt 2:1) to give the title compound (39.9 mg, 66%) as colorless oil.

¹H-NMR (CDCl₃): 0.60 (6H, t, J=7.26 Hz), 0.85 (3H, t, J=7.41 Hz), 0.97 (6H, t, J=7.25 Hz), 1.65 (4H, q, J=7.26 Hz), 1.97-2.10 (4H, m), 2.16 (3H, s), 2.22 (3H, s), 2.25-2.85 (6H, m), 4.03-4.20 (2H, m), 4.83-4.92 (1H, m), 5.11 (1H, s), 6.67 (1H, d, J=8.08 Hz), 6.85-7.00 (5H, m).

### (6) Preparation of (S)-5-(4-{1-[4-((E)-1,3-Diethyl-3-hydroxy-pent-1-enyl)-3-methyl-phenyl]-1-ethyl-propyl}-2-methyl-phenoxy)-4-hydroxy-pentanoic acid

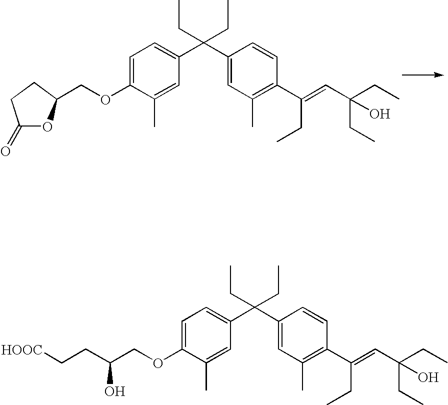

To a solution of (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one (compound prepared in Example 247-(5)) (39.9 mg, 0.0787 mmol) in MeOH (0.854 ml) was added 1N KOH solution (0.427 ml) and the reaction mixture was stirred at room temperature for 1 h. The mixture was concentrated under reduced pressure and obtained residure was purified by preparative TLC (CHCl₃:MeOH=8:3, saturated with H₂O) to give the title compound (41.8 mg, quant) as colorless oil.

¹H-NMR (CDCl₃): 0.60 (6H, t, J=7.09 Hz), 0.85 (3H, t, J=7.42 Hz), 0.97 (6H, t, J=7.26 Hz), 1.65 (4H, q, J=7.08 Hz), 1.90-2.05 (6H, m), 2.18 (3H, s), 2.22 (3H, s), 2.62 (4H, m), 3.85 (1H, t, J=9.23 Hz), 3.97 (1H, d, J=9.23 Hz), 4.10 (1H, m), 5.11 (1H, s), 6.68 (1H, d, J=8.24 Hz), 6.94 (5H, m); MS (ESI+): 542 ([M+NH₄]⁺).

### Example 248

### Preparation of (S)-3-(4-{1-Ethyl-1-[4-((E)-3-ethyl-3-hydroxy-1-methyl-pent-1-enyl)-3-methyl-phenyl]-propyl}-2-methyl-phenoxy)-propane-1,2-diol

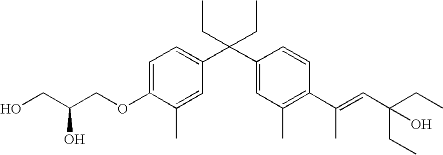

### (1) Preparation of (E)-5-(4-{1-[4-((R)-2,2-Dimethyl-[1,3]dioxolan-4-ylmethoxy)-3-methyl-phenyl]-1-ethyl-propyl}-2-methyl-phenyl)-3-ethyl-hex-4-en-3-ol

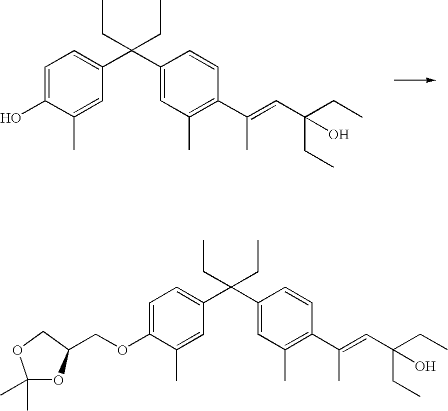

To a solution of 4-{1-Ethyl-1-[4-((E)-3-ethyl-3-hydroxy-1-methyl-pent-1-enyl)-3-methyl-phenyl]-propyl}-2-methyl-phenol (compound prepared in Example 246-(2)) (15 mg, 0.038 mmol) in DMF(1 ml) were added K₂CO₃(10.5 mg, 0.076 mmol) and (S)-2,2-Dimethyl-1,3-dioxolane-4-ylmethyl p-toluenesulfonate(21.8 mg, 0.076 mmol) and the mixture was stirred at 100 degrees C. for 7 h. The reaction mixture was poured into sat. NaHCO₃ aq. and the products were extracted with AcOEt. The extracts were washed with water and brine, dried over MgSO₄, filtered and concentrated under reduced pressure. The obtained residue was chromatographed on silica gel with EtOAc-n-Hexane (1:2) to give the title compound (13.1 mg, 68%) as colorless oil.

¹H-NMR (CDCl₃): 0.60 (6H, t, J=7.26 Hz), 0.97 (6H, t, J=7.26 Hz), 1.40 (3H, s,), 2.17 s), 1.46 (3H, s), 1.66 (4H, q, J=7.25 Hz), 1.97-2.08 (4H, m), 2.11 (3H, s), 2.17 (3H, s), 2.23 (3H, s), 3.88-4.20 (4H, m), 4.40-4.50 (1H, m), 5.18 (1H, s), 6.69 (1H, d, J=8.25 Hz), 6.85-7.00 (5H, m).

### (2) Preparation of (S)-3-(4-{1-Ethyl-1-[4-((E)-3-ethyl-3-hydroxy-1-methyl-pent-1-enyl)-3-methyl-phenyl]-propyl}-2-methyl-phenoxy)-propane-1,2-diol

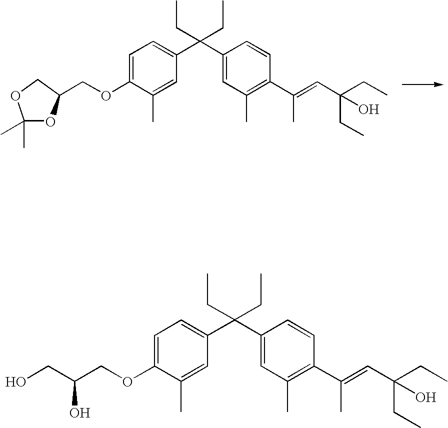

To a solution of (E)-5-(4-{1-[4-((R)-2,2-Dimethyl-[1,3]dioxolan-4-ylmethoxy)-3-methyl-phenyl]-1-ethyl-propyl}-2-methyl-phenyl)-3-ethyl-hex-4-en-3-ol (compound prepared in Example 248-(1))(7.0 mg, 0.0138 mmol) in THF/H₂O=10/1 (0.2 ml) was added CSA(6.8 mg, 0.0292 mmol) and the mixture was stirred at room temperature for 12 h. The reaction mixture was poured into sat. NaHCO₃ aq. and the products were extracted with CH₂Cl₂. The extracts were dried over MgSO₄, filtered and concentrated under reduced pressure. The obtained residue was chromatographed on silica gel with EtOAc-n-Hexane (1:2) to give the title compound (2.0 mg, 31%) as colorless oil.

¹H-NMR (CDCl₃): 0.61 (6H, t, J=7.09 Hz), 0.97 (6H, t, J=7.41 Hz), 1.62 (4H, q, J=7.59 Hz), 2.03 (4H, q, J=7.42 Hz), 2.11 (3H, s), 2.18 (3H, s), 2.23 (3H, s), 2.54 (1H, d, J=4.78 Hz), 3.74-3.86 (2H, m), 4.03-4.12 (3H, m), 5.18 (1H, s), 6.70 (1H, d, J=8.41 Hz), 6.90-6.94 (5H, m).

### Example 249

### Preparation of (S)-3-(4-{1-[4-((E)-1,3-Diethyl-3-hydroxy-pent-1-enyl)-3-methyl-phenyl]-1-ethyl-propyl}-2-methyl-phenoxy)-propane-1,2-diol

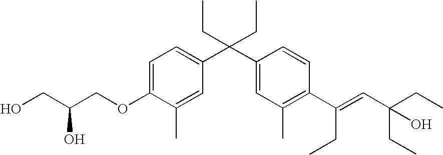

### (1) Preparation of (E)-5-(4-{1-[4-((R)-2,2-Dimethyl-[1,3]dioxolan-4-ylmethoxy)-3-methyl-phenyl]-1-ethyl-propyl}-2-methyl-phenyl-3-ethyl-hept-4-en-3-ol

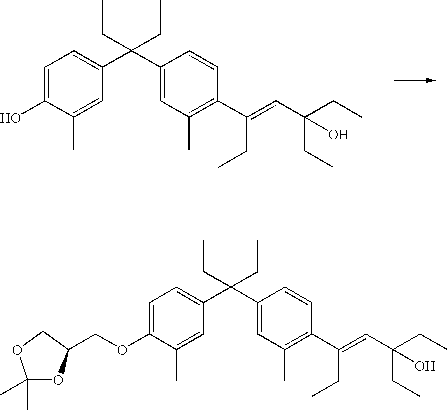

To a solution of 4-{1-[4-((E)-1,3-Diethyl-3-hydroxy-pent-1-enyl)-3-methyl-phenyl]-1-ethyl-propyl}-2-methyl-phenol (compound prepared in Example 247-(4)) (50 mg, 0.122 mmol) in DMF (1 ml) were added K₂CO₃ (33.9 mg, 0.245 mmol) and (S)-2,2-Dimethyl-1,3-dioxolane-4-ylmethyl p-toluenesulfonate (70.2 mg, 0.245 mmol) and the mixture was stirred at 100 degrees C. overnight. The reaction mixture was poured into sat. NaHCO₃ aq. and the products were extracted with AcOEt. The extracts were washed with water and brine, dried over MgSO₄, filtered and concentrated under reduced pressure. The obtained residue was purified by preparative TLC (Hex:AcOEt 2:1) to give the title compound (35.8 mg, 56%) as colorless oil.

¹H-NMR (CDCl₃): 0.60 (6H, t, J=7.25 Hz), 0.85 (3H, t, J=7.41 Hz), 0.97 (6H, t, J=7.25 Hz), 1.40 (3H, s), 1.46 (3H, s), 1.65 (4H, q, J=7.26 Hz) 1.97-2.02 (4H, m), 2.17 (3H, s), 2.22 (3H, s), 2.64 (2H, q, J=7.42 Hz), 3.90-4.20 (4H, m), 4.40-4.50 (1H, m), 5.11 (1H, s), 6.69 (1H, d, J=8.41 Hz), 6.88-6.96 (5H, m).

### (2) Preparation of (S)-3-(4-{1-[4-((E)-1,3-Diethyl-3-hydroxy-pent-1-enyl)-3-methyl-phenyl]-1-ethyl-propyl}-2-methyl-phenoxy)-propane-1,2-diol

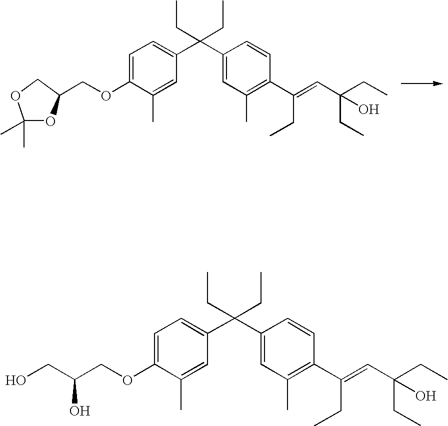

To a solution of (E)-5-(4-{1-[4-((R)-2,2-Dimethyl-[1,3]dioxolan-4-ylmethoxy)-3-methyl-phenyl]-1-ethyl-propyl}-2-methyl-phenyl)-3-ethyl-hept-4-en-3-ol (compound prepared in Example 249-(1)) (32.7 mg, 0.063 mmol) in THF/H₂O=10/1(0.94 ml) was added CSA(31 mg, 0.133 mmol) and the mixture was stirred at room temperature for 12 h. The reaction mixture was poured into sat. NaHCO₃ aq. and the products were extracted with CH₂Cl₂. The extracts were dried over MgSO₄, filtered and concentrated under reduced pressure. The obtained residue was chromatographed on silica gel with EtOAc-n-Hexane (1:2) to give the title compound (16.0 mg, 53%) as colorless oil.

¹H-NMR (CDCl₃): 0.60 (6H, t, J=7.14 Hz), 0.85 (3H, t, J=7.41 Hz), 0.97 (6H, t, J=7.41 Hz), 1.64 (4H, q, J=7.41 Hz) 2.04 (4H, q, J=7.41 Hz), 2.18 (3H, s), 2.22 (3H, s), 2.57 (1H, d, J=4.94 Hz), 2.64 (4H, q, J=7.41 Hz), 3.74-3.87 (2H, m), 4.03-4.14 (3H, m), 5.11 (1H, s), 6.71 (1H, d, J=8.23 Hz), 6.88-6.99 (5H, m).

### Example 250

### Preparation of (S)-3-(4-{1-Ethyl-1-[4-(3-ethyl-3-hydroxy-1-methyl-pentyl)-3-methyl-phenyl]-propyl}-2-methyl-phenoxy)-propane-1,2-diol

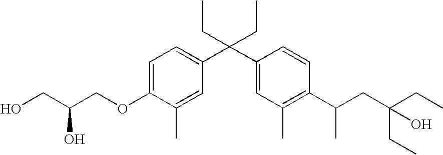

### (1) Preparation of (E)-3-(4-{1-[4-(2,2-Dimethyl-propionyloxy)-3-methyl-phenyl]-1-ethyl-propyl}-2-methyl-phenyl)-but-2-enoic acid ethyl ester

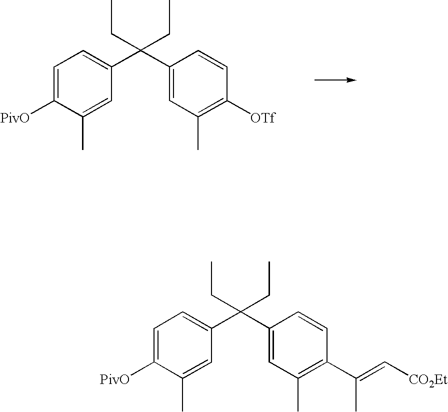

To a solution of 2,2-Dimethyl-propionic acid 4-[1-ethyl-1-(3-methyl-4-trifluoromethanesulfonyloxy-phenyl)-propyl]-2-methyl-phenyl ester(compound prepared-in Example 247-(1)) (562.3 mg, 1.12 mmol) in DMF(3.7 ml) were added Crotonic acid ethyl ester (0.557 ml, 4.50 mmol), NaHCO₃ (396 mg, 4.71 mmol), dppp(46 mg, 0.110 mmol), LiBr(67 mg, 0.771 mmol) and PdCl₂ (PPh₃)₂ (79 mg, 0.113 mmol) and the mixture was stirred at 140 degrees C. for 9 h. The reaction mixture was cooled to room temperature, poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:4) to give the title compound (176.8 mg, 34%) as colorless oil.

¹H-NMR (CDCl₃): 0.61 (6H, t, J=7.08 Hz), 0.97 (3H, t, J=7.41 Hz), 1.37 (9H, s), 2.06 (4H, q, J=7.41 Hz), 2.13 (3H, s), 2.21 (3H, s), 2.95 (4H, q, J=7.09 Hz), 3.73 (3H, s), 5.69 (1H, s), 6.85-7.01 (6H, m).

### (2) Preparation of 2,2-Dimethyl-propionic acid 4-{1-ethyl-1-[4-((E)-3-ethyl-3-hydroxy-1-methyl-pent-1-enyl)-3-methyl-phenyl]-propyl}-2-methyl-phenyl ester

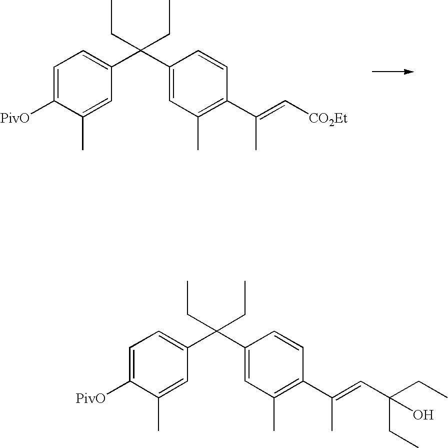

To a solution of (E)-3-(4-{1-[4-(2,2-Dimethyl-propionyloxy)-3-methyl-phenyl]-1-ethyl-propyl}-2-methyl-phenyl)-but-2-enoic acid ethyl ester (compound prepared in Example 250-(1)) (177 mg, 0.381 mmol) in THF(5 ml) was added EtMgBr (3M in Et₂O, 0.381 ml, 1.143 mmol) at 0 degrees C. and the mixture was stirred at room temperature for 2 h. The reaction mixture was poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:2) to give the title compound (123 mg, 68%) as colorless oil.

¹H-NMR (CDCl₃): 0.61 (6H, t, J=7.26 Hz), 0.97 (6H, t, J=7.41 Hz), 1.36 (9H, s), 1.66 (4H, q, J=7.41 Hz), 2.00-216 (10H, m), 2.23 (3H, s), 5.17 (1H, s), 6.82-7.05 (6H, m).

### (3) Preparation of 2,2-Dimethyl-propionic acid 4-{1-ethyl-1-[4-(3-ethyl-3-hydroxy-1-methyl-pentyl)-3-methyl-phenyl]-propyl}-2-methyl-phenyl ester

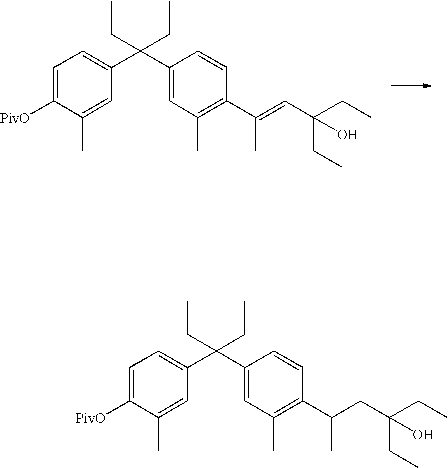

To a solution of 2,2-Dimethyl-propionic acid 4-{1-ethyl-1-[4-((E)-3-ethyl-3-hydroxy-1-methyl-pent-1-enyl)-3-methyl-phenyl]-propyl}-2-methyl-phenyl ester (compound prepared in Example 250-(2))(123 mg, 0.257 mmol) in EtOH(5 ml) was added Pd(OH)₂(20 mg) and the mixture was stirred at room temperature under H₂ atmosphere overnight. The reaction mixture was filtered through Celite and concentrated under reduced pressure to give the title compound (115 mg, 93%) as colorless oil.

¹H-NMR (CDCl₃): 0.58 (6H, t, J=7.25 Hz), 1.72-1.85 (6H, m), 1.15-1.45 (16H, m), 1.65-1.75 (1H, m), 1.89-2.13 (8H, m), 2.30 (3H, s), 3.10-3.20 (1H, m), 6.80-7.12 (6H, m).

### (4) Preparation of 4-{1-Ethyl-1-[4-(3-ethyl-3-hydroxy-1-methyl-pentyl)-3-methyl-phenyl]-propyl}-2-methyl-phenol

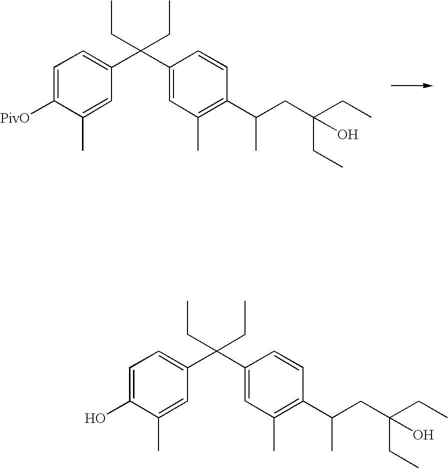

To a solution of 2,2-Dimethyl-propionic acid 4-{1-ethyl-1-[4-(3-ethyl-3-hydroxy-1-methyl-pentyl)-3-methyl-phenyl]-propyl}-2-methyl-phenyl ester (compound prepared in Example 250-(3)) (115 mg, 0.238 mmol) in MeOH(3 ml) was added 1N—KOH (0.476 ml, 0.476 mmol) and the mixture was stirred at 60 degrees C. for 1 h. The reaction mixture was poured into sat. NH₄Cl aq. and the products were extracted with CH₂Cl₂. The extracts were washed with brine, dried over MgSO₄, filtered and concentrated under reduced pressure. The obtained residue was chromatographed on silica gel with EtOAc-n-Hexane (1:2) to give the title compound (75.6 mg, 80%) as colorless oil.

¹H-NMR (CDCl₃): 0.57 (6H, t, J=7.25 Hz), 0.73-0.83 (6H, m), 1.19 (3H, d, J=6.93 Hz), 1.34-1.45 (4H, m), 1.66-1.73 (1H, m), 1.91-2.05 (5H, m), 2.17 (3H, s), 2.30 (3H, s), 3.08-3.20 (1H, m), 5.05 (1H, brs), 6.61 (1H, d, J=8.25 Hz), 6.82-6.96 (4H, m), 7.11 (1H, d, J=8.07 Hz).

### (5) Preparation of 5-(4-{1-[4-((R)-2,2-Dimethyl-[1,3]dioxolan-4-ylmethoxy)-3-methyl-phenyl]-1-ethyl-propyl}-2-methyl-phenyl)-3-ethyl-hexan-3-ol

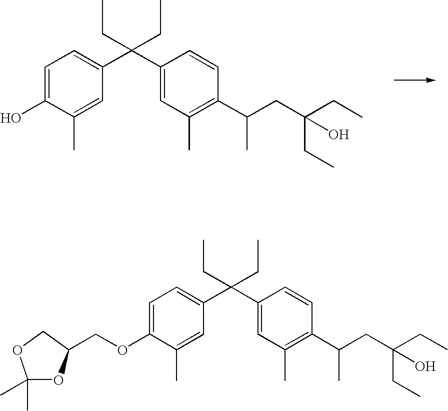

To a solution of 4-{1-Ethyl-1-[4-(3-ethyl-3-hydroxy-1-methyl-pentyl)-3-methyl-phenyl]-propyl}-2-methyl-phenol (compound prepared in Example 250-(4)) (31.1 mg, 0.078 mmol) in DMF(0.8 ml) were added K₂CO₃(21.7 mg, 0.156 mmol) and (S)-2,2-Dimethyl-1,3-dioxolane-4-ylmethyl p-toluenesulfonate (44.7 mg, 0.156 mmol) and the mixture was stirred at 100 degrees C. overnight. The reaction mixture was poured into sat. NaHCO₃ aq. and the products were extracted with AcOEt. The extracts were washed with water and brine, dried over MgSO₄, filtered and concentrated under reduced pressure. The obtained residue was purified by preparative TLC (Hex:AcOEt 2:1) to give the title compound (22.6 mg, 57%) as colorless oil.

¹H-NMR (CDCl₃): 0.57 (6H, t, J=7.26 Hz), 0.73-0.82 (6H, m), 1.19 (3H, d, J=6.93 Hz), 1.30-1.40 (4H, m), 1.40 (3H, s), 1.46 (3H, s), 1.65-1.72 (1H, m), 1.88-2.08 (5H, m), 2.15 (3H, s), 2.31 (3H, s), 3.10-3.21 (1H, s), 3.88-4.18 (4H, m), 4.41-4.50 (1H, m), 6.68 (1H, d, J=8.40 Hz), 6.89-6.95 (4H, m), 7.11 (1H, d, J=8.08 Hz).

### (6) Preparation of (S)-3-(4-{1-Ethyl-1-[4-(3-ethyl-3-hydroxy-1-methyl-pentyl)-3-methyl-phenyl]-propyl}-2-methyl-phenoxy)-propane-1,2-diol

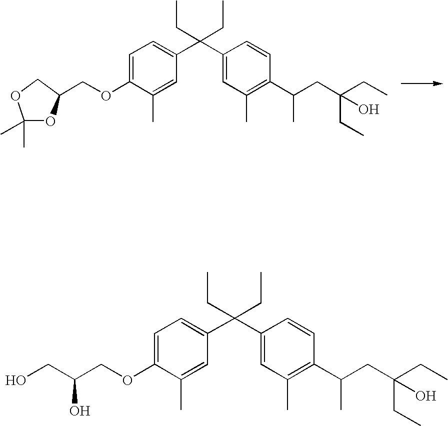

To a solution of 5-(4-{1-[4-((R)-2,2-Dimethyl-[1,3]dioxolan-4-ylmethoxy)-3-methyl-phenyl]-1-ethyl-propyl}-2-methyl-phenyl)-3-ethyl-hexan-3-ol (compound prepared in Example 250-(5))(22.6 mg, 0.044 mmol) in THF-H₂O(10:1, 1.1 ml) was added CSA(3.1 mg, 0.013 mmol) and the mixture was stirred at 60 degrees C. for 5 h. The reaction mixture was poured into sat. NaHCO₃ aq. and the products were extracted with CH₂Cl₂. The extracts were dried over MgSO₄, filtered and concentrated under reduced pressure. The obtained residue was chromatographed on silica gel with EtOAc-n-Hexane (1:1) to give the title compound (13.8 mg, 67%) as colorless oil.

¹H-NMR (CDCl₃): 0.58 (6H, t, J=7.26 Hz), 0.73-0.82 (6H, m), 1.19 (3H, d, J=6.93 Hz), 1.30-1.44 (4H, m), 1.65-1.73 (1H, m), 1.90-2.08 (5H, m), 2.16 (3H, s), 2.31 (3H, s), 3.08-3.22 (1H, m), 3.72-3.90 (2H, m), 4.00-4.18 (3H, m), 6.69 (1H, d, J=8.41 Hz), 6.89-6.95 (4H, m), 7.11 (1H, d, J=8.07 Hz); MS (ESI+) :488 ([M+NH₄]⁺).

### Example 251

### Preparation of (S)-5-(4-{1-Ethyl-1-[4-(3-ethyl-3-hydroxy-1-methyl-pentyl)-3-methyl-phenyl]-propyl}-2-methyl-phenoxy)-4-hydroxy-pentanoic acid

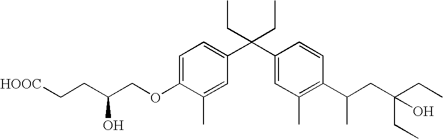

### (1) Preparation of (S)-5-(4-{1-Ethyl-1-[4-(3-ethyl-3-hydroxy-1-methyl-pentyl)-3-methyl-phenyl]-propyl}-2-methyl-phenoxymethyl)-dihydro-furan-2-one

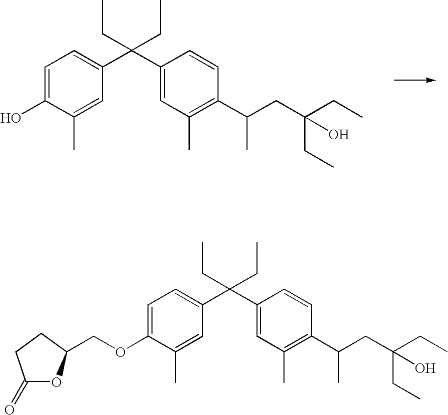

To a solution of 4-{1-Ethyl-1-[4-(3-ethyl-3-hydroxy-1-methyl-pentyl)-3-methyl-phenyl]-propyl}-2-methyl-phenol (compound prepared in Example 250-(4)) (44.5 mg, 0.112 mmol) in DMF(1 ml) were added K₂CO₃(31 mg, 0.224 mmol) and (S)-Dihydro-5-(toluenesulfonylmethyl)-2-(3H)furanone (60.5 mg, 0.224 mmol) and the mixture was stirred at 100 degrees C. overnight. The reaction mixture was poured into sat. NaHCO₃ aq. and the products were extracted with AcOEt. The extracts were washed with water and brine, dried over MgSO₄, filtered and concentrated under reduced pressure. The obtained residue was purified by preparative TLC (Hex:AcOEt 2:1) to give the title compound (39.9 mg, 51%) as colorless oil.

¹H-NMR (CDCl₃): 0.57 (6H, t, J=7.26 Hz), 0.73-0.82 (6H, m), 1.19 (3H, d, J=6.93 Hz), 1.32-1.45 (4H, m), 1.65-1.73 (1H, m), 1.90-2.10 (5H, m), 2.15 (3H, s), 2.31 (3H, s), 2.30-2.82 (4H, m), 3.10-3.23 (1H, m), 4.03-4.19 (2H, m), 4.85-4.92 (1H, m), 6.65 (1H, d, J=8.41 Hz), 6.88-6.95 (4H, m), 7.11 (1H, d, J=8.07 Hz).

### (2) Preparation of (S)-5-(4-{1-Ethyl-1-[4-(3-ethyl-3-hydroxy-1-methyl-pentyl)-3-methyl-phenyl]-propyl}-2-methyl-phenoxy)-4-hydroxy-pentanoic acid

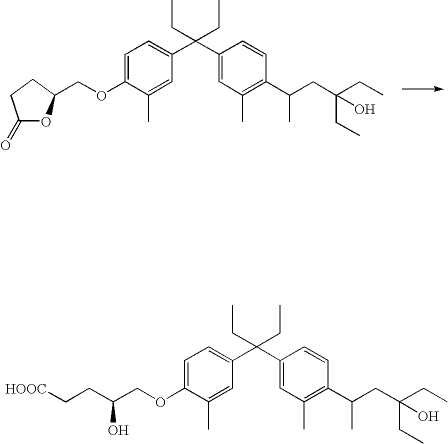

To a solution of (S)-5-(4-{1-Ethyl-1-[4-(3-ethyl-3-hydroxy-1-methyl-pentyl)-3-methyl-phenyl]-propyl}-2-methyl-phenoxymethyl)-dihydro-furan-2-one (compound prepared in Example 251-(1)) (28.4 mg, 0.057 mmol) in MeOH (1 ml) was added 1N—KOH(0.3 ml) and the mixture was stirred at room temperature for 1h. The mixture was concentrated under reduced pressure and obtained residure was purified by preparative TLC (CHCl₃:MeOH=8:3, saturated with H₂O) to give the title compound (13.3 mg, 46%) as colorless oil.

¹H-NMR (CDCl₃): 0.58 (6H, t, J=7.26 Hz), 0.73-0.82 (6H, m), 1.19 (3H, d, J=6.76 Hz), 1.32-1.47 (4H, m), 1.65-1.72 (1H, m), 1.80-2.10 (7H, m), 2.17 (3H, s), 2.31 (3H, s), 2.61 (2H, t, J=6.59 Hz), 3.08-3.21 (1H, m), 3.80-3.90 (1H, d, J=3.95-4.15 (2H, m), 6.66 (1H, d, J=8.57 Hz), 6.91-6.94 (4H, m), 7.11 (1H, d, J=8.08 Hz); MS (ESI−): 511 ([M−H]⁻).

### Example 252

### Preparation of 4-{1-Ethyl-1-[4-((E)-3-ethyl-3-hydroxy-pent-1-enyl)-3-methyl-phenyl]-propyl}-2-methyl-benzoic acid

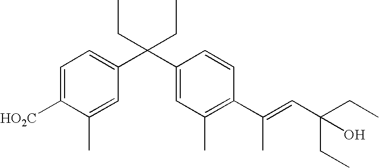

### (1 ) Preparation of Trifluoromethanesulfonic acid 4-{1-ethyl-1-[4-((E)-3-ethyl-3-hydroxy-pent-1-enyl)-3-methyl-phenyl]-propyl}-2-methyl-phenyl ester

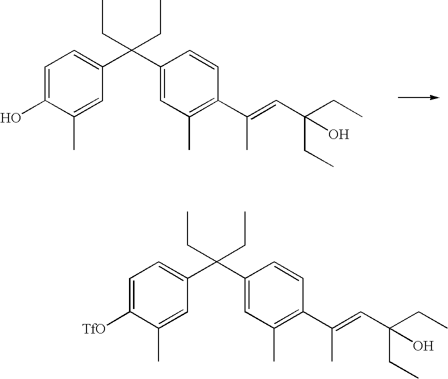

A stirred solution of 4-{1-Ethyl-1-[4-((E)-3-ethyl-3-hydroxy-pent-1-enyl)-3-methyl-phenyl]-propyl}-2-methyl-phenol (compound prepared in Example 1-(5)) (490 mg, 1.29 mmol) in CH₂Cl₂ (13 ml), pyridine (0.208 ml, 2.58 mmol) and trifluoromethanesulfonic anhydride (0.212 ml, 1.29 mmol) were added and the mixture was stirred at room temperature for 1 h. To the reaction mixture, H₂O was added. The organic layer was separated and concentrated in vacuo. The obtained residue was chromatographed on silica gel (ethyl acetate/hexane=0/100 to 15/85) to give the title compound (491 mg, 74%).

¹H-NMR: 0.61 (t, 6H), 0.92 (t, 6H), 1.65 (q, 4H), 2.07 (q, 4H), 2.32 (s, 3H), 2.32 (s, 3H), 6.02 (d, 1H), 6.75 (d, 1H), 6.90-6.92 (m, 2H), 7.03-7.12 (m, 3H), 7.31 (d, 1H).

### (2) Preparation of 4-{1-Ethyl-1-[4-((E)-3-ethyl-3-hydroxy-pent-1-enyl)-3-methyl-phenyl]-propyl}-2-methyl-benzoic acid methyl ester

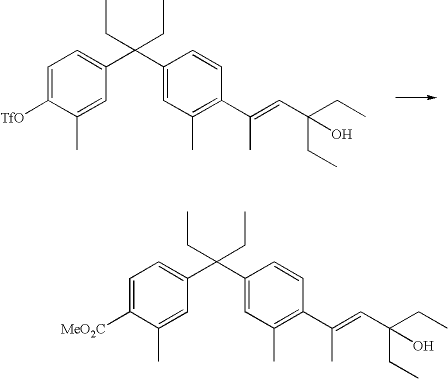

To a solution of Trifluoromethanesulfonic acid 4-{1-ethyl-1-[4-((E)-3-ethyl-3-hydroxy-pent-1-enyl)-3-methyl-phenyl]-propyl}-2-methyl-phenyl ester (compound prepared in Example 252-(1)) (461 mg, 0.90 mmol) in methanol (5 ml) and DMF (9 ml) was added triethylamine (0.376 ml, 2.70 mmol) and Pd(PPh₃)₄ (1.04 g, 0.90 mmol) and the mixture was stirred for 2 days at 60 degrees C. under CO atmosphere. The reaction mixture was separated with ethyl acetate and H₂O, the organic phase was washed with brine three times, dried over MgSO₄, concentrated in vacuo. The obtained residue was chromatographed on silica gel (ethyl acetate/hexane=0/100 to 15/85) to give the title compound (299 mg, 79%).

¹H-NMR: 0.62 (t, 6H), 0.92 (t, 6H), 1.64 (dq, 4H), 2.10 (q, 4H), 2.30 (s, 3H), 2.30 (s, 3H), 2.55 (s, 3H), 3.86 (s, 3H), 6.03 (d, 1H), 6.74 (d, 1H), 6.91-7.06 (m, 4H), 7.30 (d, 1H), 7.79 (d, 1H).

### (3) Preparation of 4-{1-Ethyl-1-[4-((E)-3-ethyl-3-hydroxy-pent-1-enyl)-3-methyl-phenyl]-propyl}-2-methyl-benzoic acid

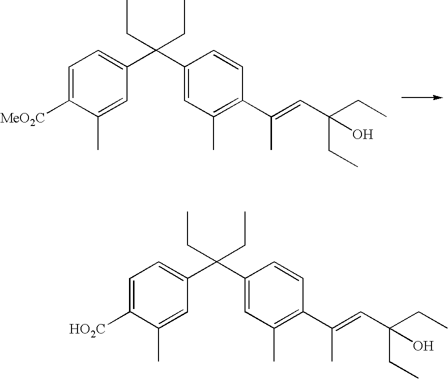

To a solution of 4-{1-Ethyl-1-[4-((E)-3-ethyl-3-hydroxy-pent-1-enyl)-3-methyl-phenyl]-propyl}-2-methyl-benzoic acid methyl ester (compound prepared in Example 252-(2)) (209 mg, 0.49 mmol) in MeOH (2 ml) and THF (2 ml) was added 1N KOH solution (3 ml) and stirred for 4 h at 60 degrees C. The reaction mixture was separated with ethyl acetate and aqueous KHSO₄, the organic phase was washed with brine, dried over MgSO₄, concentrated in vacuo to give the title compound (199.2 mg, 99%).

¹H-NMR: 0.63 (t, 6H), 0.92 (t, 6H), 1.64 (d, 4H), 2.11 (q, 4H), 2.31 (s, 3H), 2.60 (s, 3H), 6.02 (d, 1H), 6.75 (d, 1H), 6.91-7.09 (m, 4H), 7.31 (d, 1H), 7.93 (d, 1H). MS (ESI+): 390 ([M−H₂O]⁺)

### Example 253

### Preparation of 3-(4-{1-Ethyl-1-[4-((E)-3-ethyl-3-hydroxy-pent-1-enyl)-3-methyl-phenyl]-propyl}-2-methyl-benzoylamino)-propionic acid

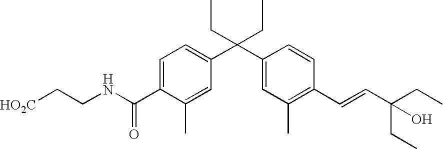

### Preparation of 3-(4-{1-Ethyl-1-[4-((E)-3-ethyl-3-hydroxy-pent-1-enyl)-3-methyl-phenyl]-propyl}-2-methyl-benzoylamino)-propionic acid

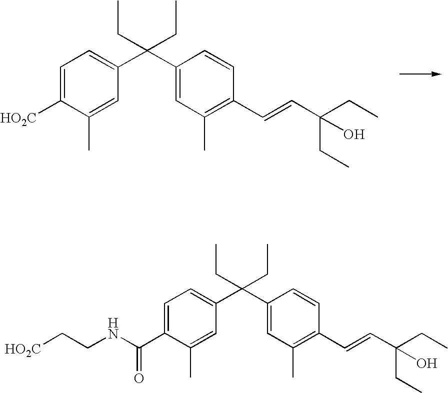

To a solution of 4-{1-Ethyl-1-[4-((E)-3-ethyl-3-hydroxy-pent-1-enyl)-3-methyl-phenyl]-propyl}-2-methyl-benzoic acid (compound prepared in Example 252-(3)) (27 mg, 0.066 mmol) in CH₂Cl₂ (0.7 ml) and triethylamine (0.028 ml, 0.198 mmol) was added beta-Ala methyl ester hydrochloride (28 mg, 0.198 mmol), WSCI hydrochloride (38 mg, 0.198 mmol) and HOBt monohydrate (10 mg, 0.066 mmol), and the mixture was stirred for 16 h at room temperature. To the reaction mixture, H₂O and CH₂Cl₂ were added. The organic layer was separated, concentrated in vacuo. The resulting residue was dissolved in THF (1 ml) and MeOH (1 ml). To the mixture, 1N aqueous KOH (1 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo. The obtained residue was chromatographed on silica gel (CH₂Cl₂/EtOH=10/1) to give the title compound (25.9 mg, 82%).

¹H-NMR: 0.61 (t, 6H), 0.91 (t, 6H), 1.64 (dq, 4H), 2.07 (q, 4H), 2.30 (s, 3H), 2.38 (s, 3H), 2.70 (t, 2H), 3.66-3.76 (m, 2H), 6.01 (d, 1H), 6.37 (t, 1H), 6.73 (d, 1H), 6.90-7.02 (m, 4H), 7.22 (d, 1H), 7.29 (d, 1H). MS (ESI+): 480 ([M+H]⁺)

### Example 254

### Preparation of 4-(4-{1-Ethyl-1-[4-((E)-3-ethyl-3-hydroxy-pent-1-enyl)-3-methyl-phenyl]-propyl}-2-methyl-benzoylamino)-butyric acid

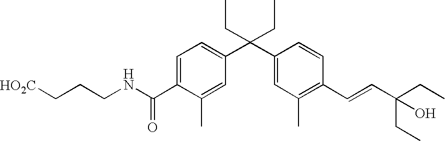

### Preparation of 4-(4-{1-Ethyl-1-[4-((E)-3-ethyl-3-hydroxy-pent-1-enyl)-3-methyl-phenyl]-propyl}-2-methyl-benzoylamino)-butyric acid

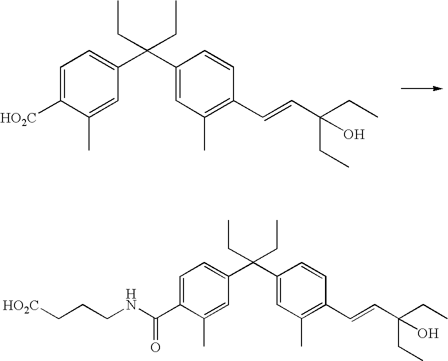

To a solution of 4-{1-Ethyl-1-[4-((E)-3-ethyl-3-hydroxy-pent-1-enyl)-3-methyl-phenyl]-propyl}-2-methyl-benzoic acid(compound prepared in Example 252-(3)) (34 mg, 0.08 mmol) in CH₂Cl₂ (0.8 ml) and triethylamine (0.067 ml, 0.48 mmol) was added gamma-aminobuthylic acid methyl ester hydrochloride (37 mg, 0.24 mmol), WSCI hydrochloride (46 mg, 0.24 mmol) and HOBt monohydrate (12 mg, 0.08 mmol), and the mixture was stirred for 18 h at room temperature. To the reaction mixture, H₂O and CH₂Cl₂ were added. The organic layer was separated, concentrated in vacuo. The resulting residue was dissolved in THF (1 mL) and MeOH (1 ml). 1N aqueous KOH (1 ml) was added and the mixture was stirred for 1 h at room temperature. The reaction mixture was concentrated in vacuo, separated with ethyl acetate and aqueous KHSO₄, the organic phase was washed with brine, dried over MgSO₄, concentrated in vacuo. The obtained residue was chromatographed on silica gel (CH₂Cl₂/EtOH=10/1) to give the title compound (30.8 mg, 75%).

¹H-NMR: 0.61 (t, 6H), 0.92 (t, 6H), 1.64 (q, 4H), 1.89-1.99 (m, 1H), 2.07 (q, 4H), 2.30 (s, 3H), 2.40 (s, 3H), 2.47 (t, 2H), 2.50 (q, 2H), 5.98-6.04 (m, 2H), 6.74 (d, 1H), 6.93-7.03 (m, 4H), 7.23 (d, 1H), 7.29 (d, 1H). MS (ESI+): 494 ([M+H]⁺⁾

### Example 255

### Preparation of N-(4-Amino-butyl)-4-{1-ethyl-1-[4-((E)-3-ethyl-3-hydroxy-pent-1-enyl)-3-methyl-phenyl]-propyl}-2-methyl-benzamide

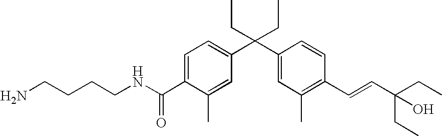

### Preparation of N-(4-Amino-butyl)-4-{1-ethyl-1-[4-((E)-3-ethyl-3-hydroxy-pent-1-enyl)-3-methyl-phenyl]-propyl}-2-methyl-benzamide

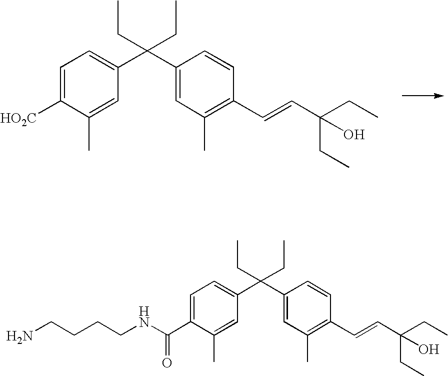

To a solution of 4-{1-Ethyl-1-[4-((E)-3-ethyl-3-hydroxy-pent-1-enyl)-3-methyl-phenyl]-propyl}-2-methyl-benzoic acid (compound prepared in Example 252-(3)) (31.5 mg, 0.077 mmol) in CH₂Cl₂ (0.8 ml) and triethylamine (0.064 ml, 0.46 mmol) was added (4-Amino-butyl)-carbamic acid 9H-fluoren-9-ylmethyl ester hydrochloride (80 mg, 0.23 mmol), WSCI hydrochloride (44 mg, 0.23 mmol) and HOBt monohydrate (12 mg, 0.077 mmol), and the mixture was stirred for 16 h at room temperature. To the reaction mixture, H₂O and CH₂Cl₂ were added. The organic layer was separated, concentrated in vacuo. The resulting residue was dissolved in DMF (0.8 ml), diethylamine (0.08 ml, 0.77 mmol) was added and the mixture was stirred for 3 h at room temperature. The reaction mixture was separated with ethyl acetate and H₂O, the organic phase was washed with H₂O twice, dried over MgSO₄, concentrated in vacuo. The obtained residue was chromatographed on silica gel (ethyl acetate/EtOH=10/1) to give the title compound (18.3 mg, 50%).

¹H-NMR: 0.61 (t, 6H), 0.92 (t, 6H), 1.49-1.58 (m, 4H), 1.64 (dq, 4H), 2.07 (q, 4H), 2.30 (s, 3H), 2.40 (s, 3H), 2.75 (t, 2H), 3.44 (q, 2H), 6.01 (d, 1H), 6.05-6.07 (m, 1H), 6.74 (d, 1H), 6.90-7.02 (m, 4H), 7.22 (d, 1H), 7.29 (d, 1H); MS (ESI+): 479 ([M+H]⁺).

### Example 256

### Preparation of 1-(4-{1-Ethyl-1-[3-methyl-(S)-4-(tetrahydro-furan-2-ylmethoxy)-phenyl]-propyl}-2-methyl-phenyl)-4,4-dimethyl-pentan-3-ol

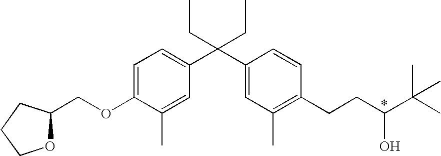

### Preparation of 5(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-dihydro-furan-2-one

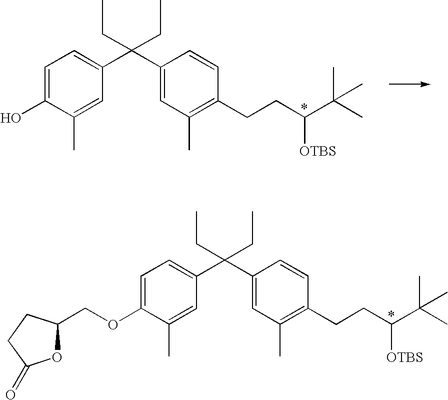

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 187-(1)) (140 mg, 0.28 mmol) in DMF (1.4 ml), (S)-(+)-dihydro-5-(p-tolylsulfonyloxymethyl)-2(3H)-furanone (228 mg, 0.84 mmol), and K₂CO₃ (195 mg, 1.41 mmol) were added at room temperature and the mixture was stirred at room temperature for 8 h. To the mixture, ethyl acetate was added and the mixture was washed with 30% NaH₂PO₄ aq. and brine, dried over MgSO₄, concentrated in vacuo. The obtained residue was chromatographed on silica gel (AcOEt/hexane=1/2 to 2/1) to give the title compound (140 mg, 36%) as colorless oil.

¹H-NMR (CDCl₃): 0.07 (s, 3H), 0.10 (s, 3H), 0.59 (t, 6H, J=7.3 Hz), 0.84 (s, 9H), 0.93 (s, 9H), 1.5-1.9 (m, 2H), 2.04 (q, 4H, J=7.3 Hz), 2.16 (s, 3H), 2.24 (s, 3H), 2.32-2.48 (m, 4H), 2.50-2.82 (m, 2H), 3.34 (dd, 1H, J=3.2, 7.2 Hz), 4.12 (ddd, 2H, J=3.5, 10.3, 13.7 Hz), 4.85-4.95 (m, 1H), 6.66 (d, 1H, J=8.2 Hz), 6.85-7.00 (m, 5H);

### (2) Preparation of tert-Butyl-{1-[2-(4-{1-ethyl-1-[3-methyl-(S)-4-(tetrahydro-furan-2-ylmethoxy)-phenyl]-propyl}-2-methyl-phenyl)-ethyl]-2,2-dimethyl-propoxy}-dimethyl-silane

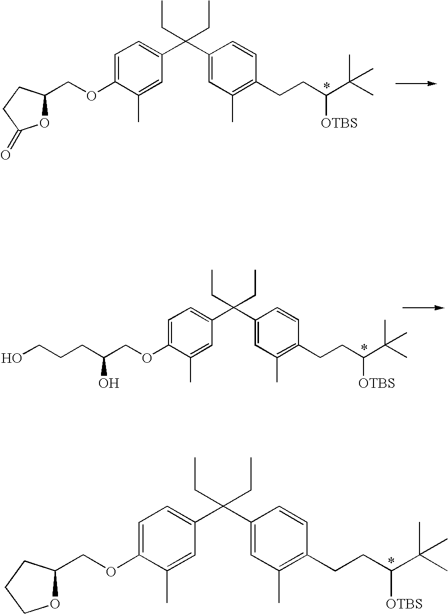

To a solution of 5(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-dihydro-furan-2-one (compound prepared in Example 256-(1))(62 mg, 0.1 mmol) in Et₂O (1 ml), LiAlH₄ (6 mg, 0.16 mmol) was added at 0 degrees C. and the mixture was stirred at room temperature for 2 h. To the mixture, ethyl acetate and brine were added and the mixture was stirred at room temperature for 0.5 h, dried over MgSO₄, concentrated in vacuo to give 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-(4S)-pentane-1,4-diol (60 mg) as colorless oil.

To a solution of 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-(4S)-pentane-1,4-diol (45 mg, 0.075 mmol) in THF (0.5 ml), PPh₃ (20 mg, 0.076 mmol) and DEAD (0.014 ml, 0.077 mmol) were added at 0 degrees C. and the mixture was stirred at room temperature for 2 h. To the mixture, Et₂O was added and the mixture was washed with water and brine, dried over MgSO₄, concentrated in vacuo. The obtained residue was chromatographed on silica gel (AcOEt/hexane=1/10) to give the title compound (22 mg, 50%) as colorless oil.

¹H-NMR (CDCl₃): 0.07 (s, 3H), 0.10 (s, 3H), 0.60 (t, 6H, J=7.3 Hz), 0.88 (s, 9H), 0.93 (s, 9H), 1.50-1.60 (m, 1H), 1.80-2.08 (m, 10H), 2.18 (s, 3H), 2.23 (s, 3H), 2.41 (dt, 1H, J=4.5, 12.9 Hz), 2.76 (dt, 1H, J=5.6, 12.6 Hz), 3.34 (dd, 1H, J=3.2, 7.2 Hz), 3.80-4.10 (m, 4H), 4.20-4.40 (m, 1H), 6.68 (d, 1H, J=8.1 Hz), 6.91-6.99 (m, 5H).

### (3) Preparation of 1-(4-{1-Ethyl-1-[3-methyl-(S)-4-(tetrahydro-furan-2-ylmethoxy)-phenyl]-propyl}-2-methyl-phenyl)-4,4-dimethyl-pentan-3-ol

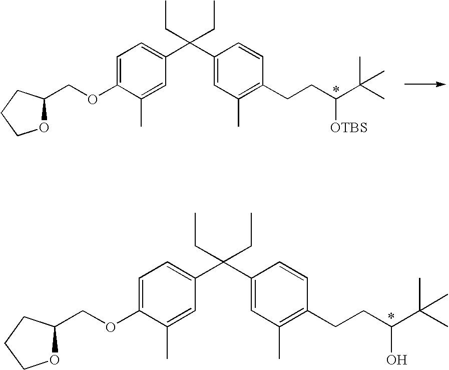

To a solution of tert-Butyl-{1-[2-(4-{1-ethyl-1-[3-methyl-(S)-4-(tetrahydro-furan-2-ylmethoxy)-phenyl]-propyl}-2-methyl-phenyl)-ethyl]-2,2-dimethyl-propoxy}-dimethyl-silane (compound prepared in Example 256-(2)) (20 mg, 0.034 mmol) in THF (1 ml), TBAF (1 M in THF, 0.5 ml, 0.5 mmol) was added at room temperature and the mixture was refluxed for 3 h. To the mixture, Et₂O was added and the mixture was washed with water, dried over MgSO₄, concentrated in vacuo. The obtained residue was chromatographed on silica gel (AcOEt/hexane=1/2) to give the title compound (8 mg, 51%) as colorless oil.

¹H-NMR (CDCl₃): 0.59 (t, 6H, J=7.3 Hz), 0.89 (s, 9H), 1.26 (t, 3H, J=7.2 Hz), 1.30-1.60 (m, 1H), 1.70-2.15 (m, 10H), 2.18 (s, 3H), 2.25 (s, 3H), 2.50-2.65 (m, 1H), 2.80-2.90 (m, 1H), 3.20-4.00 (m, 4H), 4.20-4.40 (m, 1H), 6.68 (d, 1H, J=8.2 Hz), 6.90-6.95 (m, 4H), 7.02 (d, H, J=8.6 Hz); MS (ESI+): 484 ([M+NH₄]⁺).

### Example 257

### 5-(4-{1-Ethyl-1-[4-(3-hydroxy-4,4-dimethyl-pentyl)-3-methyl-phenyl]-propyl)-2-methyl-phenoxy)-4-oxo-pentanoic acid

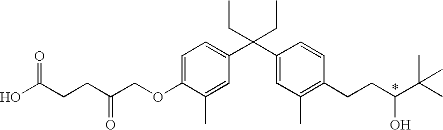

### (1) Preparation of 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-4(S)-hydroxy-pentanoic acid methyl ester

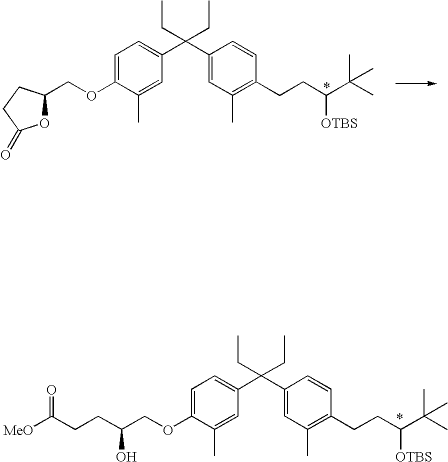

To a solution of (S)-5-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-dihydro-furan-2-one (compound prepared in Example 256-(1)) (60 mg, 0.1 mmol) in THF (0.5 ml) and MeOH (1 ml), 1N KOH aq. (0.2 ml, 0.2 mmol) was added at room temperature and the mixture was stirred at room temperature for 2 h. To the mixture, ethyl acetate was added and the mixture was washed with 30% NaH₂PO₄ aq. and brine, dried over MgSO₄, concentrated in vacuo, and chromatographed on silica gel (AcOEt/hexane=1/2) to give carboxylic acid. To a solution of the carboxylic acid in toluene (0.5 ml) and MeOH (0.5 ml), TMSCHN₂ (1M in Et₂O) was added at room temperature and the mixture was stirred at room temperature for 2 h. The reaction mixture was concentrated in vacuo and the obtained residue was chromatographed on silica gel (AcOEt/hexane=1/2) to give the title compound (60 mg, 95%) as colorless oil.

¹H-NMR (CDCl₃): 0.07 (s, 3H), 0.11 (s, 3H), 0.61 (t, 6H, J=7.2 Hz), 0.89 (s, 9H), 0.94 (s, 9H), 1.50-1.65 (m, 1H), 1.70-1.96 (m, 3H), 2.02 (q, 4H, J=7.1 Hz), 2.18 (s, 3H), 2.24 (s, 3H), 2.30-2.60 (m, 3H), 2.70-2.85 (m, 1H), 3.30-3.40 (m, 1H), 3.70 (s, 3H), 3.80-4.10 (m, 2H), 4.10 (brs, 1H), 6.68 (d, 1H, J=8.1 Hz), 6.90-7.10 (m, 5H);

### (2) Preparation of 5-(4-{1-Ethyl-1-[4-(3-hydroxy-4,4-dimethyl-pentyl)-3-methyl-phenyl]-propyl}-2-methyl-phenoxy)-4-oxo-pentanoic acid

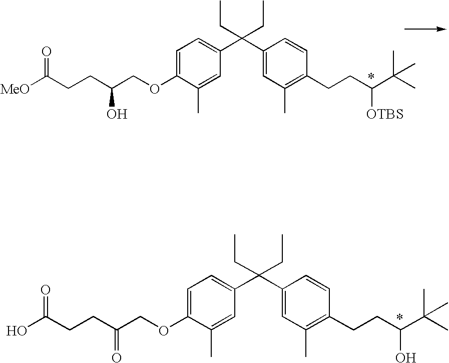

To a solution of 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]4(S)-hydroxy-pentanoic acid methyl ester (compound prepared in Example 257-(1))(60 mg, 0.1 mmol) in dichloromethane (1 ml), Dess-Martin reagent (50 mg, 0.12 mmol) was added at 0 degrees C. and the mixture was stirred at room temperature for 1 h. To the mixture, ethyl acetate was added and the mixture was washed with saturated NaHCO₃ aq., water, and brine, dried over MgSO₄, concentrated in vacuo, and the obtained residue was chromatographed on silica gel (AcOEt/hexane=1/2) to give 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-4-oxo-pentanoic acid methyl ester (58 mg, 97%) as colorless oil.

To a solution of 5-[4-(1-{4-[3-(tert-Butyl-dimethyl-silanyloxy)-4,4-dimethyl-pentyl]-3-methyl-phenyl)-1-ethyl-propyl)-2-methyl-phenoxy]-4-oxo-pentanoic acid methyl ester (58 mg, 0.1 mmol) in THF (1 ml), TBAF (1 M in THF, 1 ml, 1 mmol) was added at 0 degrees C. and the mixture was refluxed for 4 h. To the mixture, ethyl acetate was added and the mixture was washed with 30% NaH₂PO₄ aq., dried over MgSO₄, concentrated in vacuo, and the obtained residue was chromatographed on silica gel (AcOEt/hexane=5/1) to give the title compound (11 mg, 23%) as colorless oil.

¹H-NMR (CDCl₃): 0.59 (t, 6H, J=7.3 Hz), 0.89 (s, 9H), 1.40-1.60 (m, 1H), 1.70-1.90 (m, 1H), 2.04 (q, 4H, J=7.1 Hz), 2.24 (s, 3H), 2.25 (s, 3H), 2.50-2.61 (m, 1H), 2.71 (t, 2H, J=6.4 Hz), 2.80-3.30 (m, 3H), 4.12 (dd, 1H, J=7.2, 14.3 Hz), (brs, 2H), 6.56 (d, 1H, J=9.1 Hz), 6.80-7.10 (m, 5H); MS (ESI+): 519 ([M+Na]⁺).

### Example 258

### Preparation of 5-(4-{1-Ethyl-1-[4-(3-hydroxy-4,4-dimethyl-pentyl)-3-methyl-phenyl]-propyl)-2-methyl-phenoxy)-pentane-1,4-diol

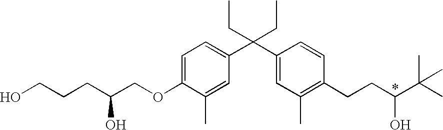

### Preparation of 5-(4-{1-Ethyl-1-[4-(3-hydroxy-4,4-dimethyl-pentyl)-3-methyl-phenyl]-propyl}-2-methyl-phenoxy)-(4S)-pentane-1,4-diol

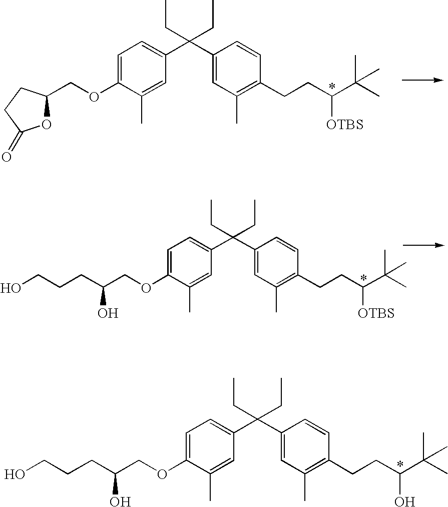

To a solution of 5(S)-[4-(1-{4-[3-(tert-Butyl-dimethyl-silanyloxy)-4,4-dimethyl-pentyl]-3-methyl-phenyl}-1-ethyl-propyl)-2-methyl-phenoxymethyl]-dihydro-furan-2-one (compound prepared in Example 256-(1)) (62 mg, 0.1 mmol) in Et₂O (1 ml), LiAlH₄ (6 mg, 0.16 mmol) was added at 0 degrees C. and the mixture was stirred at room temperature for 2 h. To the mixture, ethyl acetate and brine were added and the mixture was stirred at room temperature for 0.5 h, dried over MgSO₄, concentrated in vacuo to give 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-(4S)-pentane-1,4-diol (60 mg) as colorless oil.

To a solution of 5-[4-(1-{4-[3-(tert-Butyl-dimethyl-silanyloxy)-4,4-dimethyl-pentyl]-3-methyl-phenyl}-1-ethyl-propyl)-2-methyl-phenoxy]-(4S)-pentane-1,4-diol (15 mg, 0.075 mmol) in THF (1 ml), TBAF (1M in THF, 1 mmol) was added at room temperature and the mixture was refluxed for 4 h. To the mixture, ethyl acetate was added and the mixture was washed with 30% NaH₂PO₄ aq., dried over MgSO₄, concentrated in vacuo, and the obtained residue was chromatographed on silica gel (AcOEt/hexane=5/1) to give the title compound (11 mg, 90%) as colorless oil.

¹H-NMR (CDCl₃): 0.60 (t, 6H, J=7.3 Hz), 0.93 (s, 9H), 1.40-1.90 (m, 6H), 2.04 (q, 4H, J=7.4 Hz), 2.18 (s, 3H), 2.26 (s, 3H), 2.50-2.65 (m, 1H), 2.80-2.95 (m, 1H), 3.25 (dd, 1H, J=1.6, 10.4 Hz), 3.65-3.80 (m, 2H), 3.84 (dd, 1H, J=7.4, 9.2 Hz), 3.96 (dd, 1H, J=3.6, 9.1 Hz), 4.00-4.15 (m, 1H), 6.68 (d; 1H, J=8.2 Hz), 6.85-7.00 (m, 4H), 7.02 (d, 1H, J=8.7 Hz); MS (ESI+): 485 ([M+H]⁺).

### Example 259

### Preparation of 6-(4-{1-Ethyl-1-[4-(3-hydroxy-4,4-dimethyl-pentyl)-3-methyl-phenyl]-propyl}-2-methyl-phenyl)-hex-5-ynoic acid

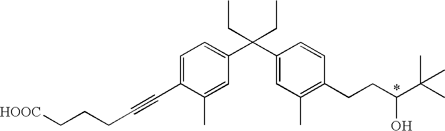

### (1) Preparation of Trifluoro-methanesulfonic acid 4-(1-{4-[3-(tert-butyl-dimethyl-silanyloxy)-4,4-dimethyl-pentyl]-3-methyl-phenyl}-1-ethyl-propyl)-2-methyl-phenyl ester

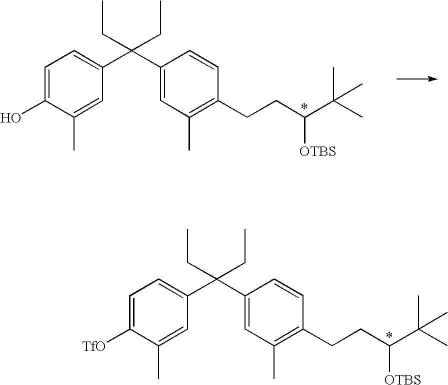

To a solution of 4-(1-{4-[3-(tert-Butyl-dimethyl-silanyloxy)-4,4-dimethyl-pentyl]-3-methyl-phenyl}-1-ethyl-propyl)-2-methyl-phenol (compound prepared in Example 187-(1)) (1.8 g, 3.6 mmol) in dichloromethane (18 ml), pyridine (0.44 ml, 5.4 mmol), and Tf₂O (0.73 ml, 4.3 mmol) were added at 0 degrees C. and the mixture was stirred at room temperature for 0.5 h. To the mixture, ethyl acetate was added and the mixture was washed with water, dried over MgSO₄, concentrated in vacuo, and the obtained residue was chromatographed on silica gel (ethyl acetate/hexane=1/10) to give the title compound (1.55 g, 68%) as colorless oil.

¹H-NMR (CDCl₃): 0.06 (s, 3H), 0.09 (s, 3H), 0.59 (t, 6H, J=7.3 Hz), 0.87 (s, 9H), 0.93 (s, 9H), 1.50-1.65 (m, 1H), 1.70-1.85 (m, 1H), 2.04 (q, 4H, J=7.3 Hz), 2.23 (s, 3H), 2.31 (s, 3H), 2.41 (dt, 1H, J=4.5, 13.0 Hz), 2.76 (dt, 1H, J=5.5, 13.0 Hz), 3.34 (dd, 1H, J=3.3, 7.0 Hz), 6.80-6.90 (m, 2H), 6.95-7.15 (m, 4H).

### (2) Preparation of Trifluoro-methanesulfonic acid 4-{1-ethyl-1-[4-(3-hydroxy-4,4-dimethyl-pentyl)-3-methyl-phenyl]-propyl}-2-methyl-phenyl ester

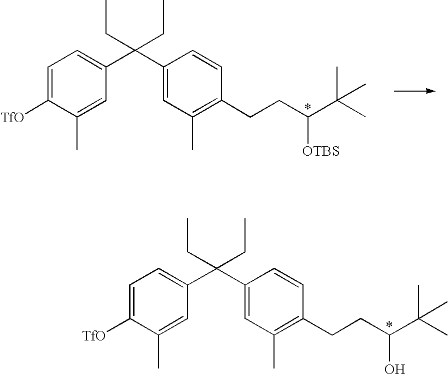

To a solution of trifluoro-methanesulfonic acid 4-(1-{4-[3-(tert-butyl-dimethyl-silanyloxy)-4,4-dimethyl-pentyl]-3-methyl-phenyl}-1-ethyl-propyl)-2-methyl-phenyl ester (compound prepared in Example 259-(1)) (95 mg, 0.15 mmol) in dichloromethane (0.75 ml), trifluoroacetic acid (0.25 ml) was added at room temperature and the mixture was stirred at room temperature for 1.5 h. The mixture was concentrated in vacuo, and chromatographed on silica gel (ethyl acetate/hexane=1/2) to give the title compound (70 mg, 90%) as colorless oil.

¹H-NMR (CDCl₃): 0.60 (t, 6H, J=7.3 Hz), 0.90 (s, 9H), 1.35-1.60 (m, 2H), 1.70-1.90 (m, 1H), 2.06 (q, 4H, J=7.3 Hz), 2.27 (s, 3H), 2.32 (s, 3H), 2.50-2.65 (m, 1H), 2.80-3.00 (m, 1H), 3.26 (brd, 1H, J=10.2 Hz), 6.80-6.95 (m, 2H), 7.00-7.20 m, 4H);

### (3) Preparation of 6-(4-{1-Ethyl-1-[4-(3-hydroxy-4,4-dimethyl-pentyl)-3-methyl-phenyl]-propyl}-2-methyl-phenyl)-hex-5-ynoic acid

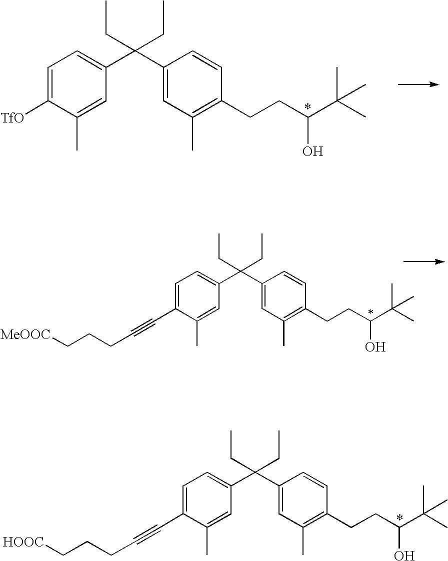

To a solution of trifluoro-methanesulfonic acid 4-(1-{4-[3-(tert-butyl-dimethyl-silanyloxy)-4,4-dimethyl-pentyl]-3-methyl-phenyl}-1-ethyl-propyl)-2-methyl-phenyl ester (compound prepared in Example 259-(2)) (50 mg, 0.1 mmol) in DMF (1 ml), PdCl₂(dppf)₂-CH₂Cl₂ (8 mg, 0.01 mmol), Et₃N (0.05 ml, 0.3 mmol), Cul (20 mg, 0.1 mmol), and hex-5-ynoic acid methyl ester (20 mg, 0.16 mmol) were added at room temperature under nitrogen and stirred at 120 degrees C. for 8 h. To the mixture, ethyl acetate was added and the mixture was washed with water and brine, dried over MgSO₄, concentrated in vacuo, and the obtained residue was chromatographed on silica gel (ethyl acetate/hexane=1/4) to give 6-(4-{1-ethyl-1-[4-(3-hydroxy-4,4-dimethyl-pentyl)-3-methyl-phenyl]-propyl}-2-methyl-phenyl)-hex-5-ynoic acid methyl ester (16 mg) as colorless oil including impurities.

To a solution of 6-(4-{1-ethyl-1-[4-(3-hydroxy-4,4-dimethyl-pentyl)-3-methyl-phenyl]-propyl}-2-methyl-phenyl)-hex-5-ynoic acid methyl ester(16 mg) in MeOH (1 ml), 1N NaOH aq. (0.2 ml, 0.2 mmol) was added at room temperature and stirred at room temperature for 4 h. To the mixture, ethyl acetate was added and the mixture was washed with 30% NaH₂PO₄ aq., dried over MgSO₄, concentrated in vacuo, and the obtained residue was chromatographed on silica gel (ethyl acetate/hexane=1/1) to give the title compound (6 mg, 13%, 2steps) as colorless oil.

¹H-NMR (CDCl₃): 0.59 (t, 6H, J=7.3 Hz), 0.89 (s, 9H), 1.40-1.60 (m, 1H), 1.70-2.00 (m, 3H), 2.05 (q, 4H, J=7.3 Hz), 2.24 (s, 3H), 2.35 (s, 3H), 2.45-2.65 (m, 5H), 2.80-2.95 (m, 1H), 3.24 (dd, 1H, J=1.5, 10.4 Hz), 6.80-7.05 (m, 5H), 7.23 (d, 1H, J=8.1 Hz); MS (ESI+): 494 ([M+NH4]⁺).

### Example 260

### Preparation of (R)-5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one

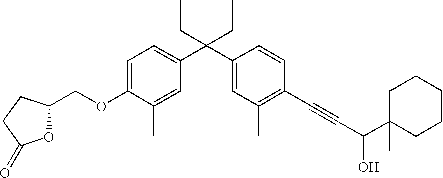

### (1) Preparation of 1-Methyl-cyclohexanecarboxylic acid N-methoxy-N-methyl-amide

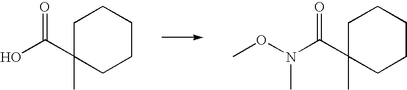

To a solution of 1-Methyl-1-cyclohexanecarboxylic acid (500 mg, 3.516 mmol) in CH₂Cl₂ (5 ml), methoxymethyl amine hydrochloride (411.6 mg, 4.219 mmol), DMAP (214.8 mg, 1.758 mmol), Et₃N (996.2 mg, 9.845 mmol) and HCl salt of WSCI (943.7 mg, 4.923 mmol) were added under nitrogen gas at 0 degrees C. and the mixture was stirred at room temperature for 17 h. To the mixture, ethyl acetate was added and the organic layer was washed with brine, dried over magnesium sulfate, concentrated in vacuo and the obtained residue was chromatographed on silica gel (dichrolomethane/methanol=90/10) to give the title compound (373.5 mg, 57.3%).

¹H-NMR (chloroform-d): 1.17 (s, 3H), 1.24-1.56 (m, 8H), 2.05-2.11 (m, 2H), 3.15 (s, 3H), 3.64 (s, 3H); MS (ESI+): 186([M+H]⁺).

### (2) Preparation of tert-Butyl-{4-[1-ethyl-1-(4-ethynyl-3-methyl-phenyl)-propyl]-2-methyl-phenoxy}-dimethyl-silane

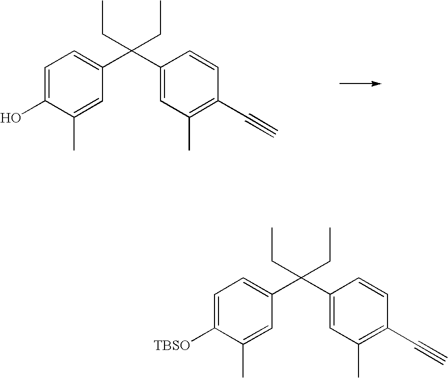

To a solution of 4-[1-Ethyl-1-(4-ethynyl-3-methyl-phenyl)-propyl]-2-methyl-phenol (compound prepared in Example 1-(3)) (639 mg, 2.185 mmol) in DMF (2.7 ml), TBSCI (658.8 mg, 4.371 mmol) and imidazole (743.9 mg, 10.926 mmol) were added at room temperature and the mixture was stirred at room temperature for 17 h. To the mixture, ethyl acetate was added and the mixture was washed with water and brine, dried over magnesium sulfate, concentrated in vacuo, and the obtained residue was chromatographed on silica gel (n-hexane/ethyl acetate=97/3 to ethyl acetate=only) to give the title compound (555.2 mg, 62.5%) as colorless oil.

¹H-NMR (chloroform-d): 0.23 (s, 6H), 0.62 (t, 6H, J=7.3 Hz), 1.03 (s, 9H), 2.06 (q, 4H, J=7.3 Hz), 2.18 (s, 3H), 2.42 (s, 3H), 3.23 (s, 1H), 6.67 (d, 1H, J=8.4 Hz), 6.84 (dd, 1H, J=2.6, 8.4 Hz), 6.90 (d, 1H, J=2.4 Hz), 6.97 (dd, 1H, J=2.0, 8.1 Hz), 7.04 (brs, 1H), 7.36 (d, 1H, J=8.2 Hz); MS (ESI+): 407([M+H]⁺).

### (3) Preparation of 3-(4-{1-[4-(tert-Butyl-dimethyl-silanyloxy)-3-methyl-phenyl]-1-ethyl-propyl}-2-methyl-phenyl)-1-(1-methyl-cyclohexyl)-prop-2-yn-1-one

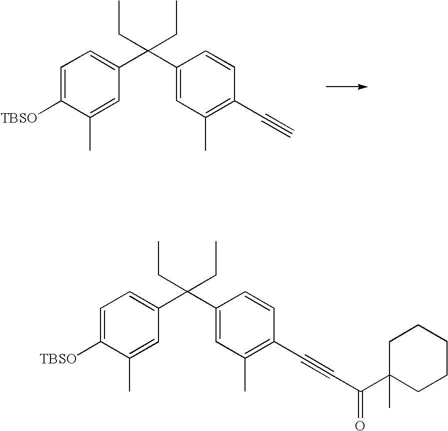

To a solution of tert-Butyl-{4-[1-ethyl-1-(4-ethynyl-3-methyl-phenyl)-propyl]-2-methyl-phenoxy}-dimethyl-silane (compound prepared in Example 260-(2)) (50 mg, 0.123 mmol) in THF (0.2 ml), n-BuLi in n-hexane (0.07 ml, 0.172 mmol) was added at −78 degrees C. under nitrogen gas and the mixture was stirred at room temperature for 20 min. And then, to this solution, 1-methyl-cyclohexanecarboxylic acid methoxy-methyl-amide (compound prepared in Example 260-(1)) (34.2 mg, 0.185 mmol) was added at −78 degrees C. under nitrogen gas and the mixture was stirred for 15 h raising the reaction temperature from −78 degrees C. to room temperature. To the mixture, ethyl acetate was added and the mixture was washed with saturated ammonium chloride solution and brine, dried over magnesium sulfate, concentrated in vacuo, and the obtained residue was chromatographed on silica gel (n-hexane/ethyl acetate=10/1) to give the title compound (41.1 mg, 62.9%). MS (ESI+): 531 ([M+H]⁺).

### (4) Preparation of 3-(4-{1-[4-(tert-Butyl-dimethyl-silanyloxy)-3-methyl-phenyl]-1-ethyl-propyl}-2-methyl-phenyl)-1-(1-methyl-cyclohexyl)-prop-2-yn-1-ol

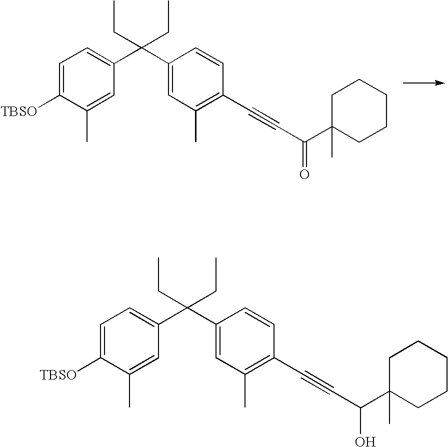

To a solution of 3-(4-{1-[4-(tert-Butyl-dimethyl-silanyloxy)-3-methyl-phenyl]-1-ethyl-propyl}-2-methyl-phenyl)-1-(1-methyl-cyclohexyl)-prop-2-yn-1-one (compound prepared in Example 260-(3)) (41.1 mg, 0.077 mmol) in THF (0.8 ml) and MeOH (0.3 ml), NaBH₄ (8.8 mg, 0.232 mmol) was added at room temperature and stirred at room temperature for 13 h. To the mixture, ethyl acetate was added and the mixture was washed with brine, dried over magnesium sulfate, concentrated in vacuo, and chromatographed on silica gel (n-hexane/ethyl acetate=30/1) to give the title compound (36.5 mg, 88.5%).

¹H-NMR (chloroform-d): 0.21 (s, 6H), 0.60 (t, 6H, J=7.4 Hz), 1.02 (s, 9H), 1.08 (s, 3H), 1.40-1.63 (m, 10H), 1.77 (brs, 1H), 2.04 (q, 4H, J=7.4 Hz), 2.15 (s, 3H), 2.39 (s, 3H), 4.35 (s, 1H), 6.63 (d, 1H, J=8.2 Hz), 6.81 (dd, 1H, J=2.5, 8.3 Hz), 6.86 (d, 1H, J=2.3 Hz), 6.94 (dd, 1H, J=1.6, 8.1 Hz), 7.00 (brs, 1H), 7.29 (d, 1H, J=8.2 Hz).

### (5) Preparation of (R)-5-[4-(1-Ethyl-1-{4-[3-hydroxy-3-(1-methyl-cyclohexyl)-prop-1-ynyl]-3-methyl-phenyl)-propyl)-2-methyl-phenoxymethyl]-dihydro-furan-2-one

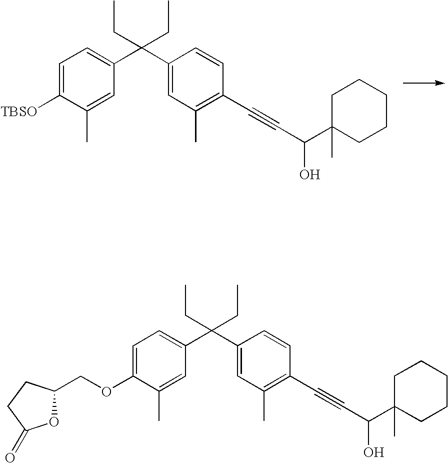

To a solution of 3-(4-{1-[4-(tert-Butyl-dimethyl-silanyloxy)-3-methyl-phenyl]-1-ethyl-propyl}-2-methyl-phenyl)-1-(1-methyl-cyclohexyl)-prop-2-yn-1-ol (compound prepared in Example 260-(4)) (36 mg, 0.068 mmol) in THF (0.7 ml), 1.0M TBAF in THF (0.1 ml, 0.1 mmol) was added at room temperature and stirred at room temperature for 15 h. To the mixture, ethyl acetate was added and the mixture was washed with diluted potassium hydrogen sulfate aq. and brine, dried over magnesium sulfate, concentrated in vacuo, and the obtained residue was chromatographed on silica gel (n-hexane/ethyl acetate=10/1 and 7/1) to give the mixture including the title compound (28.8 mg). To a solution of this mixture (10 mg) in DMF (0.2 ml), (R)-(−)-Dihydro-5-(p-tolyl-sulfonyloxymethyl)-2(3H)-furanone (16.1 mg, 0.06 mmol) and K₂CO₃ (13.2 mg, 0.096 mmol) were added at room temperature and stirred at 105 degrees C. for 11 h. To the reaction mixture, ethyl acetate was added and the mixture was washed with brine, dried over magnesium sulfate, concentrated in vacuo, and the obtained residue was chromatographed on silica gel (n-hexane/ethyl acetate=3/1) to give the title compound (4.6 mg, 37.3% for 2 steps).

¹H-NMR (chloroform-d): 0.59 (t, 6H, J=7.3 Hz), 1.07 (s, 3H), 1.40-1.64 (m, 10H), 1.74 (d, 1H, J=6.2 Hz), 2.04 (q, 4H, J=7.3 Hz), 2.15 (s, 3H), 2.26-2.63 (m, 3H), 2.39 (s, 3H), 2.78 (ddd, 1H, J=6.8, 9.7, 17.0 Hz), 4.06 (dd, 1H, J=3.5, 10.3 Hz), 4.17 (dd, 1H, J=3.3, 10.3 Hz), 4.35 (d, 1H, J=6.3 Hz), 4.864.92 (m, 1H), 6.66 (d, 1H, J=8.5 Hz), 6.87 (brd, 1H, J=1.8 Hz), 6.90-6.95 (m, 2H), 6.98 (brs, 1H), (d, 1H, J=8.1 Hz); MS (ESI+): 534([M+NH₄]⁺).

### Example 261

### Preparation of (R)-5-[4-(1-Ethyl-1-{4-[3-hydroxy-3-(1-methyl-cyclohexyl)-propyl]-3-methyl-phenyl}-propyl)-2-methyl-phenoxymethyl]-dihydro-furan-2-one

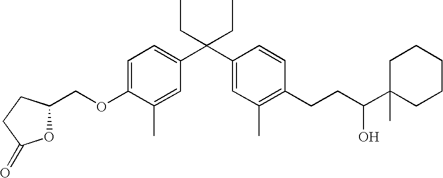

### (1) Preparation of 4-(1-Ethyl-1-{4-[3-hydroxy-3-(1-methyl-cyclohexyl)-propyl]-3-methyl-phenyl}-propyl)-2-methyl-phenol

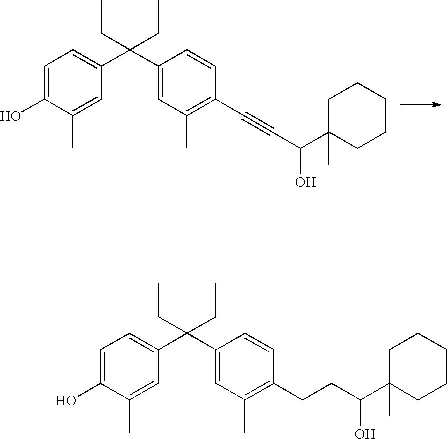

To a solution of 4-(1-Ethyl-1-{4-[3-hydroxy-3-(1-methyl-cyclohexyl)-prop-1-ynyl]-3-methyl-phenyl}-propyl)-2-methyl-phenol (compound prepared in Example 260-(5)) (18 mg, 0.043 mmol) in AcOEt (1 ml), 10% Pd—C (2 mg) was added and the mixture was stirred at room temperature under hydrogen gas for 15 h. To the mixture, ethyl acetate was added and the mixture was filtered through celite, concentrated in vacuo, and the obtained residue was chromatographed on silica gel (n-hexane/ethyl acetate=3/1 and 1/1) to give the title compound (15.5 mg, 85.3%).

¹H-NMR (chloroform-d): 0.60 (t, 6H, J=7.5 Hz), 0.85 (s, 3H), 1.23-1.59 (m, 12H), 1.75-1.84 (m, 1H), 2.04 (q, 4H, J=7.5 Hz), 2.20 (s, 3H), 2.26 (s, 3H), 2.56 (ddd, 1H, J=6.3, 10.0,13.9 Hz), 2.88 (ddd, 1H, J=5.0, 10.1, 15.1 Hz), 3.32 (brd, 1H, J=10.0 Hz), 4.71 (brs, 1H), 6.65 (d, 1H, J=8.2 Hz), 6.84-6.92 (m, 4H), 7.02 (d, 1H, J=8.6 Hz); MS(positive): 440([M+NH₄]⁺).

### (2) Preparation of (R)-5-[4-(1-Ethyl-1-{4-[3-hydroxy-3-(1-methyl-cyclohexyl)-propyl]-3-methyl-phenyl}-propyl )-2-methyl-phenoxymethyl]-dihydro-furan-2-one

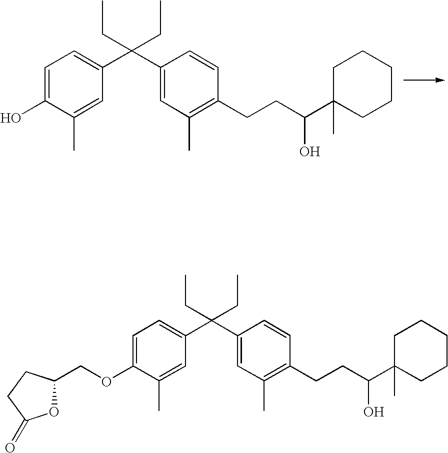

To a solution of 4-(1-Ethyl-1-{4-[3-hydroxy-3-(1-methyl-cyclohexyl)-propyl]-3-methyl-phenyl)-propyl)-2-methyl-phenol (compound prepared in Example 261-(1)) (15 mg, 0.035 mmol) in DMF (0.4 ml), toluene-4-sulfonic acid (R)-5-oxo-tetrahydro-furan-2-ylmethyl ester (24 mg, 0.089 mmol) and K₂CO₃ (19.6 mg, 0.142 mmol) was added at room temperature and the mixture was stirred at 60 degrees C. for 4 h and at 85 degrees C. for 4 h. To the mixture, ethyl acetate was added and the mixture was washed with water and brine, dried over magnesium sulfate, concentrated in vacuo and the obtained residue was chromatographed on silica gel (n-hexane/ethyl acetate=3/1 and 1/1) to give the title compound (9.3 mg, 50.4%).

¹H-NMR (chloroform-d): 0.60 (t, 6H, J=7.3 Hz), 0.85 (s, 3H), 1.16-1.65 (m, 11H), 1.73-1.87 (m, 1H), 2.04 (q, 4H, J=7.3 Hz), 2.16 (s, 3H), 2.18 (s, 3H), 2.26-2.63 (m, 4H), 2.73-2.92 (m, 2H), 3.28-3.33 (m, 1H), 4.07 (dd, 1H, J=3.5, 10.3 Hz), 4.17 (dd, 1H, J=3.4, 10.3 Hz), 4.86-4.92 (m, 1H), 6.66 (d, 1H, J=8.3 Hz), 6.89-7.03 (m, 5H); MS (ESI+): 538 ([M+NH₄]⁺).

### Example 262

### Preparation of (R)-5-[4-(1-Ethyl-1-{4-[3-hydroxy-3-(1-methyl-cyclohexyl)-prop-1-ynyl]-3-methyl-phenyl}-propyl)-2-methyl-phenoxy]-4-hydroxy-pentanoic acid

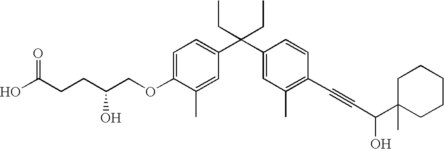

### Preparation of (R)-5-[4-(1-Ethyl-1-{4-[3-hydroxy-3-(1-methyl-cyclohexyl)-prop-1-ynyl]-3-methyl-phenyl}-propyl)-2-methyl-phenoxy]-4-hydroxy-pentanoic acid

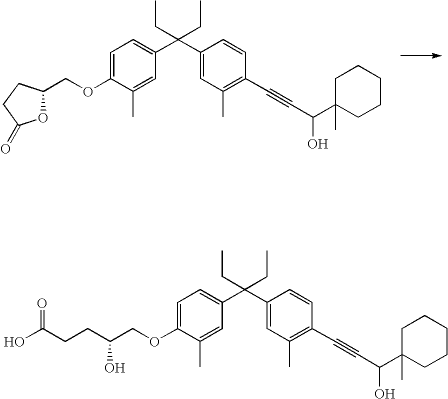

To a solution of 5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one (compound prepared in Example 260-(5)) (4 mg, 0.008 mmol) in THF (0.15 ml) and MeOH (0.05 ml), 1N KOH aq (0.05 ml, 0.05 mmol) was added at room temperature and the mixture was stirred at 65-70 degrees C. for 2 h. The mixture was concentrated in vacuo and chromatographed on silica gel (dichloromethane/methanol=10/1) to give the title compound (2.1 mg, 50.7%).

¹H-NMR (chloroform-d): 0.60 (t, 6H, J=7.3 Hz), 1.07 (s, 3H), 1.40-1.68 (m, 10H), 1.89-2.02 (m, 2H), 2.04 (q, 4H, J=7.3 Hz), 2.16 (s, 3H), 2.39 (s, 3H), 2.63 (t, 2H, J=7.2 Hz), 3.85 (dd, 1H, J=6.9, 9.2 Hz), 3.97 (dd, 1H, J=3.5, 9.2 Hz), 4.02-4.16 (m, 1H), 4.35 (s, 1H), 6.67 (d, 1H, J=8.4 Hz), 6.87-6.99 (m, 4H), 7.29 (d, 1H, J=8.1 Hz); MS (ESI−): 533([M−H]⁻).

### Example 263

### Preparation of (R)-5-[4-(1-Ethyl-1-{4-[3-hydroxy-3-(1-methyl-cyclohexyl)-propyl]-3-methyl-phenyl}-propyl)-2-methyl-phenoxy]-4-hydroxy-pentanoic acid

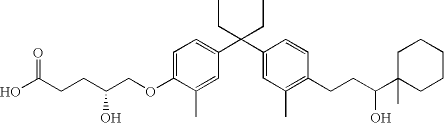

### Preparation of (R)-5-[4-(1-Ethyl-1-{4-[3-hydroxy-3-(1-methyl-cyclohexyl)-propyl]-3-methyl-phenyl}-propyl)-2-methyl-phenoxy]-4-hydroxy-pentanoic acid

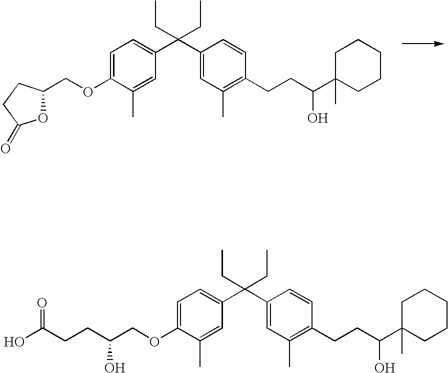

To a solution of 5-[4-(1-Ethyl-1-{4-[3-hydroxy-3-(1-methyl-cyclohexyl)-propyl]-3-methyl-phenyl}-propyl)-2-methyl-phenoxymethyl]-dihydro-furan-2-one (compound prepared in Example 261) (8.7 mg, 0.017 mmol) in THF (0.3 ml) and MeOH (0.1 ml), 1N KOH aq (0.1 ml, 0.1 mmol) was added at room temperature and the mixture was stirred at 65-70 degrees C. for 2 h. The mixture was concentrated in vacuo and chromatographed on silica gel (dichloromethane/methanol=10/1) to give the title compound (3.7 mg, 41.1%).

¹H-NMR (chloroform-d): 0.60 (t, 6H, J=7.2 Hz), 0.85 (s, 3H), 1.15-1.59 (m, 11H), 1.74-1.84 (m, 1H), 1.89-1.99 (m, 2H), 2.04 (q, 4H, J=7.3 Hz), 2.17 (s, 3H), (s, 3H), 2.51-2.60 (m, 1H), 2.62 (dt, 2H, J=1.6, 7.1 Hz), 2.87 (ddd, 1H, J=4.8, 10.1, 13.9 Hz), 3.31 (brd, 1H, J=9.1 Hz), 3.85 (dd, 1H, J=6.7, 9.2 Hz), 3.98 (dd, 1H, J=3.5, 9.2 Hz), 4.04-4.11 (m, 1H), 6.67 (d, 1H, J=8.2 Hz), 6.89-6.97 (m, 4H), 7.02 (d, 1H, J=8.6 Hz); MS (ESI−): 537([M−H]⁻).

### Example 264

### Preparation of 4-(4-{1-Ethyl-1-[4-((S)-3-hydroxy-4,4-dimethyl-pentyl)-3-methyl-phenyl]-propyl}-2-methyl-phenoxymethyl)-benzoic acid

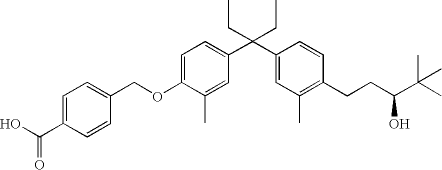

### (1) Preparation of 4-[4-(1-{4-[(S)-3-(tert-Butyl-dimethyl-silanyloxy)-4,4-dimethyl-pentyl]-3-methyl-phenyl}-1-ethyl-propyl)-2-methyl-phenoxymethyl]-benzoic acid methyl ester

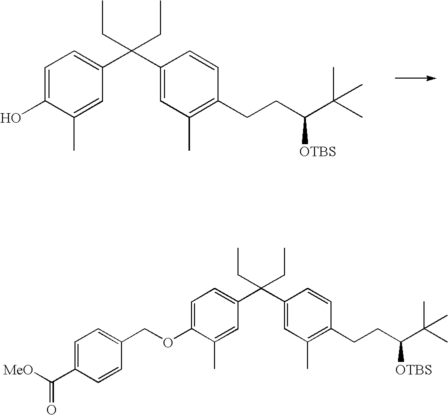

To a solution of 4-(1-{4-[(S)-3-(tert!Butyl-dimethyl-silanyloxy)-4,4-dimethyl-pentyl]-3-methyl-phenyl}-1-ethyl-propyl)-2-methyl-phenol (compound prepared in Example 187-(1)) (45 mg, 0.091 mmol) in DMF (0.9 ml), methyl 4-(bromomethyl)benzoate (52 mg, 0.226 mmol) and K₂CO₃ (50 mg, 0.362 mmol) were added at room temperature and the mixture was stirred at 110 degrees C. for 13 h. To the mixture, ethyl acetate was added and the mixture was washed with brine, dried over magnesium sulfate, concentrated in vacuo, and the obtained residue was chromatographed on silica gel (n-hexane/ethyl acetate=10/1) to give the title compound (33.6 mg, 57.5%).

¹H-NMR (chloroform-d): 0.08 (s, 3H), 0.12 (s, 3H), 0.62 (t, 6H, J=7.3 Hz), 0.89 (s, 9H), 0.95 (s, 9H), 1.52-1.65 (m, 1H), 1.74-1.85 (m, 1H), 2.06 (q, 4H, J=7.3 Hz), 2.25 (s, 6H), 2.43 (dt, 1H, J=4.7, 12.3 Hz), 2.77 (dt, 1H, J=5.3, 13.6 Hz), 3.35 (dd, 1H, J=3.2, 7.2 Hz), 3.93 (s, 3H), 5.10 (s, 2H), 6.74 (d, 1H, J=8.8 Hz), 6.91-7.01 (m, 5H), 7.52 (d, 2H, J=8.6 Hz), 8.06 (d, 2H, J=8.4 Hz).

### (2) Preparation of 4-(4-{1-Ethyl-1-[4-((S)-3-hydroxy-4,4-dimethyl-pentyl)-3-methyl-phenyl]-propyl}-2-methyl-phenoxymethyl)-benzoic acid

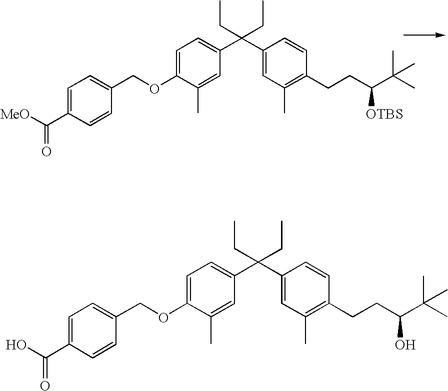

To a solution of 4-[4-(1-{4-[(S)-3-(tert-Butyl-dimethyl-silanyloxy)-4,4-dimethyl-pentyl]-3-methyl-phenyl}-1-ethyl-propyl)-2-methyl-phenoxymethyl]-benzoic acid methyl ester (compound prepared in Example 264-(1)) (33.6 mg, 0.052 mmol) in THF (0.5 ml), 1.0M TBAF in THF (0.26 ml, 0.26 mmol) was added at room temperature and the mixture was stirred at 70 degrees C. for 4 h. To the mixture, ethyl acetate was added and the mixture was washed with diluted potassium hydrogen sulfate aq. and brine, dried over magnesium sulfate, concentrated in vacuo, and the obtained residue was chromatographed on silica gel (dichloromethane/methanol=10/1 and dichloromethane/methanol=15/1) to give the title compound (16.5 mg, 61.3%).

¹H-NMR (chloroform-d): 0.61 (t, 6H, J=7.3 Hz), 0.90 (s, 9H), 1.45-1.58 (m, 1H), 1.76-1.85 (m, 1H), 2.05 (q, 4H, J=7.3 Hz), 2.25 (s, 3H), 2.26 (s, 3H), 2.56 (ddd, 1H, J=6.0, 10.1, 14.1 Hz), 2.88 (ddd, 1H, J=5.1, 10.7, 14.1 Hz), 3.26 (dd, 1H, J=1.5 Hz), 5.12 (s, 2H), 6.74 (d, 1H, J=9.1 Hz), 6.91-6.97 (m, 4H), 7.03 (d, 1H, J=7.9 Hz), 7.56 (d, 2H, J=8.2 Hz), 8.13 (d, 2H, J=8.2 Hz); MS (ESI−): 515([M−H]⁻).

### Example 265

### Preparation of 4-(4-{1-Ethyl-1-[4-(3-hydroxy-4,4-dimethyl-pent-1-ynyl)-3-methyl-phenyl]-propyl}-2-methyl-phenylcarbamoyl)-butyric acid

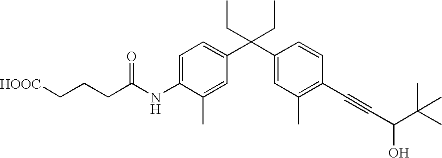

### (1) Preparation of 3-m-Tolyl-pentan-3-ol

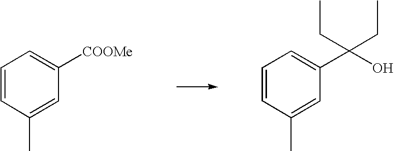

To a solution of 3-Methyl-benzoic acid methyl ester (5 g, 33.29 mmol) in THF (50 ml) at 0 degrees C. was added EtMgBr (3M in Et₂O, 33 ml) and the mixture was stirred at 0 degrees C. for 1 h. The reaction mixture was poured into sat. NH₄Cl aq. and the products were extacted with AcOEt. The extracts were washed with brine, dried over MgSO₄, filtered and concentrated under reduced pressure to give the title compound (5.67 g, 96%).

¹H NMR (CDCl₃): 0.76 (6H, t, J=7.59 Hz), 1.75-1.90 (4H, m), 2.36 (3H, s), 7.03 (1H, d, J=7.42 Hz), 7.12-7.26 (3H, m)

### (2) Preparation of 4-(1-Ethyl-1-m-tolyl-propyl)-2-methyl-phenol

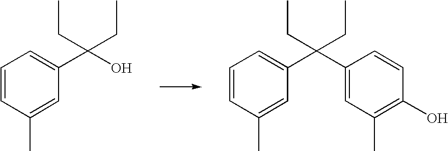

To a solution of 3-m-Tolyl-pentan-3-ol (5.57 g, 31 24 mmol) (compound prepared in Example 265-(1))in toluene (50 ml) were added cresol (12.5 ml, 93.73 mmol) and AlCl₃ (4.58 g, 34.36 mmol) and the mixture was stirred at room temperature overnight. The reaction mixture was poured into 1N—HCl and the products were extacted with CH₂Cl₂. The extracts were washed with brine, dried over MgSO₄, filtered and concentrated under reduced pressure. The obtained residue was chromatographed on silica gel with CH₂Cl₂ to give the title compound (8 g, 95%) as colorless oil.

¹H NMR (CDCl₃): 0.60 (6H, t, J=7.26 Hz), 2.00-2.09 (4H, q, J=7.25 Hz), 2.19 (3H, s), 2.29 (3H, s), 4.57 (1H, s), 6.63-7.13 (7H, m)

### (3) Preparation of Trifluoro-methanesulfonic acid 4-(1-ethyl-1-m-tolyl-propyl)-2-methyl-phenyl ester

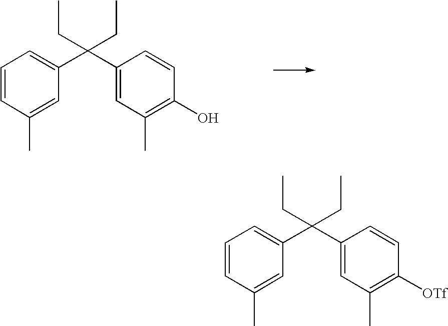

To a solution of 4-(1-Ethyl-1-m-tolyl-propyl)-2-methyl-phenol (compound prepared in Example 265-(2)) (400 mg, 1.490 mmol) were added Tf₂O (0.276 ml, 1.639 mmol) and Et₃N (0.310 ml, 2.235 mmol) and the mixture was stirred at room temperature for 30 min. The reaction mixture was poured into sat. NaHCO₃ aq. and the products were extacted with CH₂Cl₂. The extracts were washed with brine, dried over MgSO₄, filtered and concentrated under reduced pressure. The obtained residue was chromatographed on silica gel with n-Hexane-AcOEt (1:1) to give the title compound (412 mg, 69%) as colorless oil.

¹H NMR (CDCl₃): 0.60 (6H, t, J=7.25 Hz), 2.07 (4H, q, J=7.42 Hz), 2.30 (3H, s), 2.32 (3H, s), 6.92-7.18 (7H, m)

### (4) Preparation of Trifluoro-methanesulfonic acid 4-[1-ethyl-1-(3-methyl-4-nitro-phenyl)-propyl]-2-methyl-phenyl ester

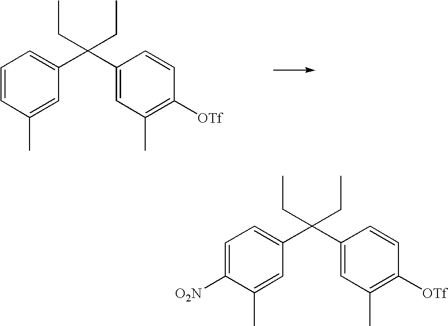

To a solution of Trifluoro-methanesulfonic acid 4-(1-ethyl-1-m-tolyl-propyl)-2-methyl-phenyl ester (compound prepared in Example 265-(3)) (100 mg, 0.250 mmol) in MeCN (3 ml) at 0 degrees C. was added NO₂BF₄ (40 mg, 0.300 mmol) and the mixture was stirred at 0 degrees C. for 15 min. The reaction mixture was poured into sat. NaHCO₃ aq. and the products were extacted with CH₂Cl₂. The extracts were washed with brine, dried over MgSO₄, filtered and concentrated under reduced pressure. The obtained residue was chromatographed on silica gel with n-Hexane-AcOEt (10:1) to give the title compound (42.2 mg, 38%) as colorless oil.

¹H NMR (CDCl₃): 0.62 (6H, t, J=7.42 Hz), 2.11 (4H, q, J=7.42 Hz), 2.33 (3H, s), 2.58 (3H, s), 6.98-7.18 (5H, m), 7.91 (1H, d, J=9.06 Hz)

### (5) Preparation of Trifluoro-methanesulfonic acid 4-[1-(4-amino-3-methyl-phenyl)-1-ethyl-propyl]-2-methyl-phenyl ester

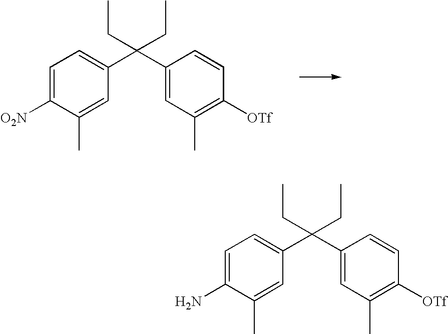

To a solution of Trifluoro-methanesulfonic acid 4-[1-ethyl-1-(3-methyl-4-nitro-phenyl)-propyl]-2-methyl-phenyl ester (compound prepared in Example 265-(4)) (130 mg, 0.292 mmol) in MeOH (3 ml) was added Pd/C (10%, 20 mg) and the mixture was stirred at room temperature under H₂ atmosphere for 1.5 h. The mixture was filtered through celite and concentrated under reduced pressure to give the title compound (113.2 mg, 93%).

¹H NMR (CDCl₃): 0.59 (6H, t, J=7.25 Hz), 2.02 (4H, q, J=7.42 Hz), 2.12 (3H, s), 2.31 (3H, s), 6.58 (1H, d, J=8.91 Hz), 6.77-6.83 (2H, m), 7.04-7.12 (3H, m)

### (6) Preparation of 4-{4-[1-Ethyl-1-(3-methyl-4-trifluoromethanesulfonyloxy-phenyl)-propyl]-2-methyl-phenylcarbamoyl}-butyric acid methyl ester

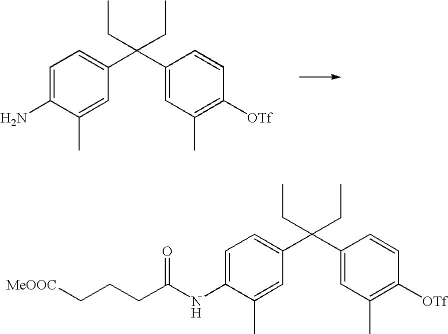

To a solution of Trifluoro-methanesulfonic acid 4-[1-(4-amino-3-methyl-phenyl)-1-ethyl-propyl]-2-methyl-phenyl ester (compound prepared in Example 265-(5)) (113 mg, 0.272 mmol) in CH₂Cl₂ (3 ml) were added glutaric acid mono methyl ester (60 mg, 0.409 mmol), EDC (104 mg, 0.544 mmol) and Et₃N (0.113 ml, 0.816 mmol) and the mixture was stirred at room temperature overnight. The reaction mixture was poured into sat. NaHCO₃ aq. and the products were extacted with CH₂Cl₂. The extracts were washed with brine, dried over MgSO₄, filtered and concentrated under reduced pressure. The obtained residue was chromatographed on silica gel with n-Hexane-AcOEt (1:1) to give the title compound (111.5 mg, 75%) as colorless oil.

¹H NMR (CDCl₃): 0.60 (6H, t, J=7.25 Hz), 2.00-2.15 (6H, m), 2.21 (3H, s), 2.31 (3H, s), 2.41-2.50 (4H, m), 3.69 (3H, s), 6.91 (1H, s), 6.97-7.15 (5H, m), 7.74 (1H, d, J=8.41 Hz)

### (7) Preparation of tert-Butyl-(1-tert-butyl-prop-2-ynyloxy)-dimethyl-silane

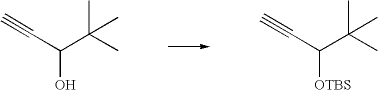

To a solution of 4,4-Dimethyl-pent-1-yn-3-ol (J. Org. Chem. 53; 20; 1988; 4736-4745) (360 mg, 3.209 mmol) in CH₂Cl₂ (10 ml) were added TBSOTf (0.884 ml, 3.851 mmol) and imidazole (437 mg, 6.418 mmol) and the mixture was stirred at room temperature for 30 min. The reaction mixture was poured into sat. NaHCO₃ aq. and the products were extacted with CH₂Cl₂. The extracts were washed with brine, dried over MgSO₄, filtered and concentrated under reduced pressure. The obtained residue was chromatographed on silica gel with n-Hexane-AcOEt (7:3) to give the title compound (314 mg, 43%) as colorless oil.

¹H NMR (CDCl₃): 0.02 (3H, s), 0.09 (3H, s), 0.91 (9H, s), 0.95 (9H, s), 2.34 (1H, s), 3.95 (1H, s)

### (8) Preparation of 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid methyl ester

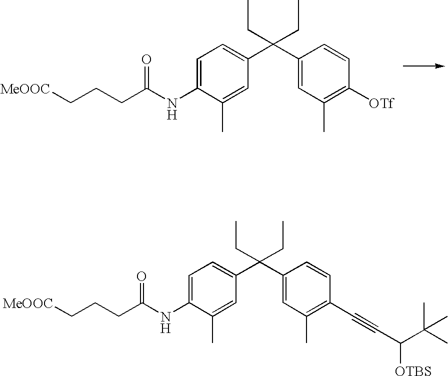

To a solution of 4-{4-[1-Ethyl-1-(3-methyl-4-trifluoromethanesulfonyloxy-phenyl)-propyl]-2-methyl-phenylcarbamoyl}-butyric acid methyl ester (compound prepared in Example 265-(6)) (111 mg, 0.204 mmol) in DMF (3 ml) were added tert-Butyl-(1-tert-butyl-prop-2-ynyloxy)-dimethyl-silane (compound prepared in Example 265-(7)) (92 mg, 0.408 mmol), PdCl₂(dppf)-CH₂Cl₂ (18 mg, 0.022 mmol), Cul (7.8 mg, 0.041 mmol) and Et₃N (0.085 ml, 0.612 mmol) and the mixture was stirred under nitrogen atmosphere at 100 degrees C. overnight. The reaction mixture was poured into sat. NH₄Cl aq. and the products were extacted with AcOEt. The extracts were washed with brine, dried over MgSO₄, filtered and concentrated under reduced pressure. The obtained residue was chromatographed on silica gel with n-Hexane-AcOEt (1:1) to give the title compound (50 mg, 40%) as colorless oil.

¹H NMR (CDCl₃): 0.10 (3H, s), 0.12 (3H, s), 0.60 (6H, t, J=7.26 Hz), 0.93 (9H, s), 1.01 (9H, s), 2.00-2.13 (6H, m), 2.20 (3H, s), 2.36 (3H, s), 2.42-2.50 (4H, m), 3.69 (3H, s), 6.90-7.07 (5H, m), 7.25 (1H, d, J=7.92 Hz), 7.71 (1H, d, J=8.24 Hz)

### (9) Preparation of 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid and 4-(4-{1-Ethyl-1-[4-(3-hydroxy-4,4-dimethyl-pent-1-ynyl)-3-methyl-phenyl]-propyl}-2-methyl-phenylcarbamoyl)-butyric acid

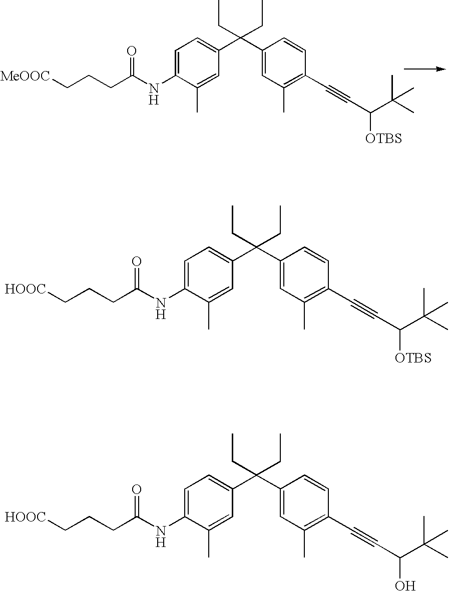

To a solution of 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid methyl ester (compound prepared in Example 265-(8)) (50 mg, 0.081 mmol) in THF-H₂O (10:1, 3.3 ml) was added CSA (56 mg, 0.243 mmol) and the mixture was stirred at 70 degrees C. for 6 h. The reaction mixture was poured into sat. NaHCO₃ aq. and the products were extacted with CH₂Cl₂. The extracts were washed with brine, dried over MgSO₄, filtered and concentrated under reduced pressure. The obtained residue was chromatographed on silica gel with CHCl₃-MeOH (10:1) to give 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid (21.4 mg, 44%) and 4-(4-{1-Ethyl-1-[4-(3-hydroxy-4,4-dimethyl-pent-1-ynyl)-3-methyl-phenyl]-propyl}-2-methyl-phenylcarbamoyl)-butyric acid (6.4 mg, 16%). 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid ; ¹H NMR (CDCl₃): 0.12 (3H, s), 0.18 (3H, s), 0.59 (6H, t, J=7.26 Hz), 0.92 (9H, s), 1.01 (9H, s), 2.00-2.15 (6H, m), 2.19 (3H, s), 2.36 (3H, s), 2.43-2.57 (4H, m), 6.95-7.05 (5H, m), 7.25 (1H, d), 7.68 (1H, d, J=8.41 Hz) 4-(4-{1-Ethyl-1-[4-(3-hydroxy-4,4-dimethyl-pent-1-ynyl)-3-methyl-phenyl]-propyl)-2-methyl-phenylcarbamoyl)-butyric acid; ¹H NMR (CDCl₃): 0.59 (6H, t, J=7,42 Hz), 1.06 (9H, s), 2.00-2.13 (6H, m), 2.18 (3H, s), 2.37 (3H, s), 2.43-2.57 (4H, m), 6.90-7.04 (5H, m), 7.28 (1H, d), 7.68 (1H, d, J=8.24 Hz); MS (ESI+): 474 ([M+H—H₂O]⁺).

### Example 266

### Preparation of 4-(4-{1-Ethyl-1-[4-(3-hydroxy-4,4-dimethyl-pentyl)-3-methyl-phenyl]-propyl}-2-methyl-phenylcarbamoyl)-butyric acid

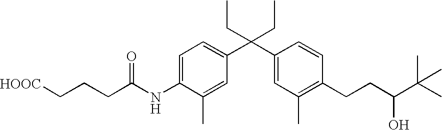

### Preparation of 4-(4-{1-Ethyl-1-[4-(3-hydroxy-4,4-dimethyl-pentyl)-3-methyl-phenyl]-propyl}-2-methyl-phenylcarbamoyl)-butyric acid methyl ester

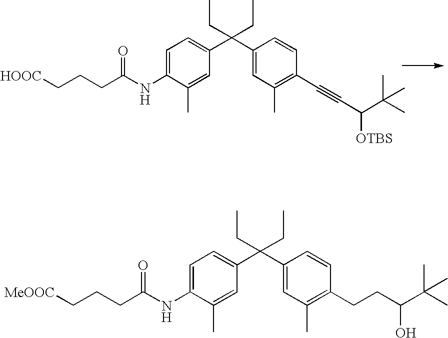

To a solution of 4-[4-(1-{4-[3-(tert-Butyl-dimethyl-silanyloxy)-4,4-dimethyl-pent-1-ynyl]-3-methyl-phenyl}-1-ethyl-propyl)-2-methyl-phenylcarbamoyl]-butyric acid (compound prepared in Example 265-(9)) (21.4 mg, 0.035 mmol) in MeOH (3 ml) was added Pd(OH)₂ (10%, 30 mg) and the mixture was stirred at room temperature under H₂ atmosphere overnight. The mixture was filtered through celite and concentrated under reduced pressure. The obtained residue was chromatographed on silica gel with CHCl₃—MeOH (10:1) to give the title compound (13.5 mg, 76%).

¹H NMR (CDCl₃): 0.60 (6H, t, J=7.26 Hz), 0.89 (9H, s), 1.45-1.55 (1H, m), 1.72-1.88 (1H, m), 2.00-2.10 (6H, m), 2.20 (3H, s), 2.25 (3H, s), 2.41-2.50 (4H, m), 2.52-2.62 (1H, m), 2.80-2.92 (1H, m), 3.20-3.30 (1H, m), 3.69 (3H, s), 6.88-7.05 (5H, m), 7.69 (1H, d, J=8.74 Hz); MS (ESI+): 510 [(M+H)+].

### (2) Preparation of 4-(4-{1-Ethyl-1-[4-(3-hydroxy-4,4-dimethyl-pentyl)-3-methyl-phenyl]-propyl}-2-methyl-phenylcarbamoyl)-butyric acid

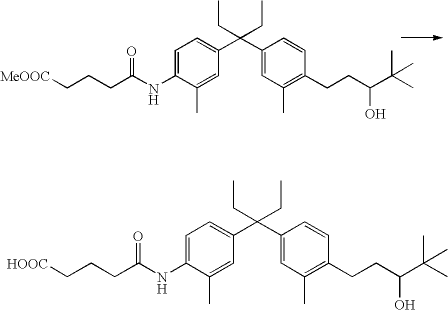

To a solution of 4-(4-{1-Ethyl-1-[4-(3-hydroxy-4,4-dimethyl-pentyl)-3-methyl-phenyl]-propyl}-2-methyl-phenylcarbamoyl)-butyric acid methyl ester (compound prepared in Example 266-(1)) (11.4 mg, 0.022 mmol) in MeOH (1 ml) was added 1N—NaOH (0.22 ml, 0.220 mmol) and the mixture was stirred at room temperature overnight. The mixture was poured into 1N—HCl and the products were extacted with CH₂Cl₂. The extracts were washed with brine, dried over MgSO₄, filtered and concentrated under reduced pressure. The obtained residue was chromatographed on silica gel with CHCl₃—MeOH (10:1) to give the title compound (7.3 mg, 67%).

¹H NMR (CDCl₃): 0.60 (6H, t, J=7.25 Hz), 0.89 (9H, s), 1.40-1.62 (1H, m), 1.70-1.90 (1H, m), 2.00-2.13 (6H, m), 2.19 (3H, s), 2.25 (3H, s), 2.44-2.62 (5H, m), 2.79-2.95 (1H, m), 3.22-3.27 (1H, m), 6.86-7.05 (5H, m), 7.65 (1H, d, J=8.24 Hz); MS (ESI+): 496 [(M+H+].

### Example 267

### Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxy]-4-hydroxy-pentanoic acid

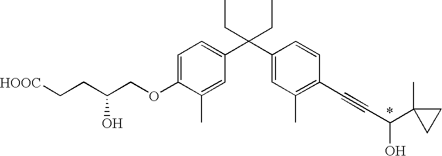

### (1) Preparation of 3-(4-{1-[4-(tert-Butyl-dimethyl-silanyloxy)-3-methyl-phenyl]-1-ethyl-propyl}-2-methyl-phenyl)-1-(1-methyl-cyclopropyl)-prop-2-yn-1-ol

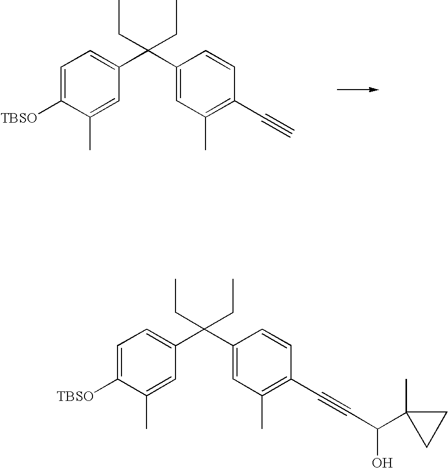

To a solution of compound prepared in Example 260-(2) (tert-Butyl-{4-[1-ethyl-1-(4-ethynyl-3-methyl-phenyl)-propyl]-2-methyl-phenoxy}-dimethyl-silane) (188 mg, 0.46 mmol) in THF (3 ml), 1.58M n-BuLi in hexane (0.35 ml, 0.55 mmol) was added at −78 degrees C. and the mixture was stirred at −78 degrees C. for 10 min. To the mixture, 1-Methyl-cyclopropanecarbaldehyde (J. Org. Chem. 64, 26, 1999, 9587-9595) (0.2 ml) was added at −78 degrees C. and the mixture was stirred at room temperature for 16 h. To the mixture, H₂O and ethyl acetate were added and the mixture was washed with H₂O, dried over MgSO₄, concentrated in vacuo and the obtained residue was chromatographed ethyl acetate/n-hexane=0/100 to 3/7) to give the title compound (126 mg, 56%).

¹H-NMR: 0.19 (s, 6H), 0.40 (q, 2H), 0.58 (t, 6H), 0.67-0.72 (m, 2H), 1.00 (s, 9H), 1.27 (s, 3H), 2.01 (q, 4H), 2.14 (s, 3H), 2.37 (s, 3H), 4.26 (d, 1H), 6.61 (d, 1H), 6.77-7.00 (m, 4H), 7.26 (s, 1H)

### (2) Preparation of 4-(1-Ethyl-1-{4-[3-hydroxy-3-(1-methyl-cyclopropyl)-prop-1-ynyl]-3-methyl-phenyl}-propyl)-2-methyl-phenol (Enantiomer A, Enantiomer B)

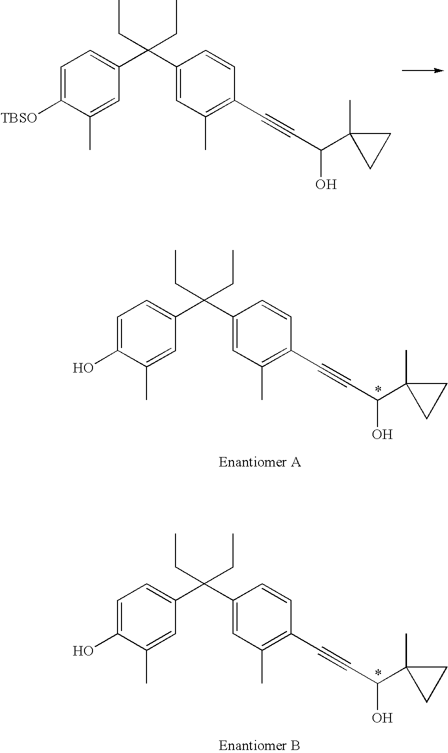

To a solution of tert-butyl-{4-[1-ethyl-1-(4-ethynyl-3-methyl-phenyl)-propyl]-2-methyl-phenoxy}-dimethyl-silane (90 mg, 0.184 mmol) in THF (2 ml), 1M TBAF in THF (0.1 ml) was added at room temperature, and the mixture was stirred at room temperature for 1 h and concentrated in vacuo. The obtained residue was chromatographed (ethyl acetate/n-hexane=0/100 to 1/1) to give the mixture of title compounds (47 mg). The racemic mixture was separated with chiral HPLC (CHIRALPAK OJ-H [DAICEL, 20 mml.D., 250 mm], ethanol/n-hexane=10/90) to obtain each enantiomer (Enantiomer A: 20 mg, 100% ee,/Enantiomer B: 15 mg, 100% ee).

Enantiomer A: Retention time: 11.874 min., Enantiomer B: Retention time: 13.291 min.

Column: CHIRALPAK OJ-H 4.6×150 mm (DAICEL)

Eluent: A: n-hexnane, B: EtOH, % B 1(0 min)—70 (15 min)

Flow rate: 0.5 ml/min.

¹H-NMR for mixture Enantiomer A and Enantiomer B: 0.41 (q, 2H), 0.59 (t, 6H), 0.67-0.72 (m, 2H), 1.27 (s, 3H), 2.02 (q, 4H), 2.18 (s, 3H), 2.37 (s, 3H), 4.26 (d, 1H), 4.67 (s, 1H), 6.64 (d, 1H), 6.80-7.00 (m, 4H), 7.26 (d, 1H); MS for mixture Enantiomer A and Enantiomer B (negative mode): 375([M−H]⁻)

### (3) Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one

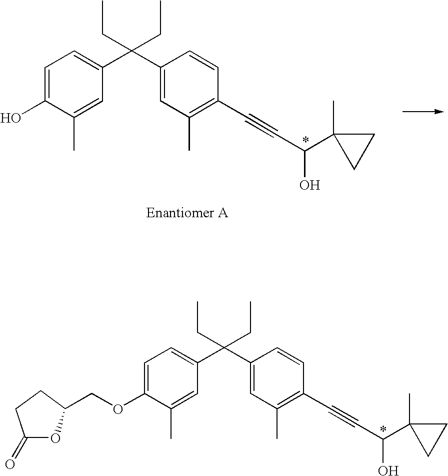

To a solution of 4-(1-ethyl-1-{4-[3-hydroxy-3-(1-methyl-cyclopropyl)-prop-1-ynyl]-3-methyl-phenyl}-propyl)-2-methyl-phenol (Enantiomer A) (10 mg, 0.0266 mmol) in DMF (0.5 ml), K₂CO3 (10 mg, 0.0725 mmol) and toluene-4-sulfonic acid (R)-5-oxo-tetrahydro-furan-2-ylmethyl ester (10 mg, 0.0756 mmol) were added at room temperature and the mixture was stirred at 90 degrees C. for 64 h. To the mixture, ethyl acetate was added and the mixture was washed with H₂O, dried over MgSO₄, concentrated in vacuo and the obtained residue was purified by preparative TLC (n-hexane:ethyl acetate=1:1 and CH₂Cl₂:MeOH=20:1) to give the title compound (6.7 mg, 53%).

¹H-NMR: 0.41 (q, 2H), 0.59 (t, 6H), 0.61-0.69 (m, 2H), 1.25 (s, 3H), 1.83 (d, 1H), 2.02 (q, 4H), 2.14 (s, 3H), 2.37 (s, 3H), 2.38-2.83 (m, 4H), 4.04-4.28 (m, 3H), 4.84-4.92 (m, 1H), 6.66 (d, 1h), 6.82-6.98 (m, 4H), 7.25 (d, 1H); MS (ESI+): 457 ([M−OH]⁺).

### (4) Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxy]-4-hydroxy-pentanoic acid

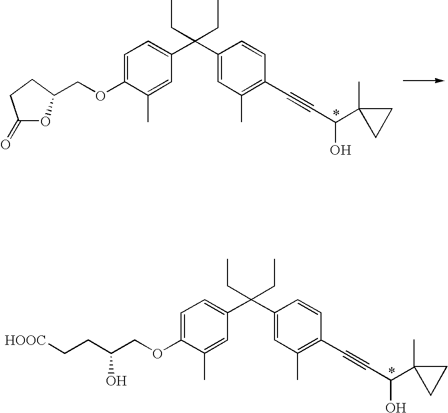

To a solution of (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one (prepared in Example 267-(3)) (6.7 mg, 0.014 mmol) in MeOH (0.5 ml) and THF (0.5 ml), 1NNaOH (0.1 ml) was added at room temperature and the mixture was stirred at room temperature for 1 h. The mixture was concentrated in vacuo and purified by preparative TLC (CHCl3:MeOH=8:3 saturated with H₂O) to give the title compound (3.3 mg, 47%).

¹H-NMR: 0.41 (q, 4H), 0.60 (t, 6H), 0.64-0.69 (m, 2H), 1.26 (s, 3H), 1.90-2.05 (m, 7H), 2.16 (s, 3H), 2.37 (s, 3H), 2.62 (t, 2H), 3.83-4.10 (m, 3H), 4.25 (s, 1H), 6.66 (s, 1H), 6.83-7.02 (m, 3H), 7.29 (s, 1H); MS. (ESI−): 491 ([M−H]⁻).

### Example 268

### Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxy]-4-hydroxy-pentanoic acid

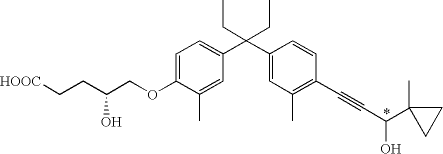

### Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one

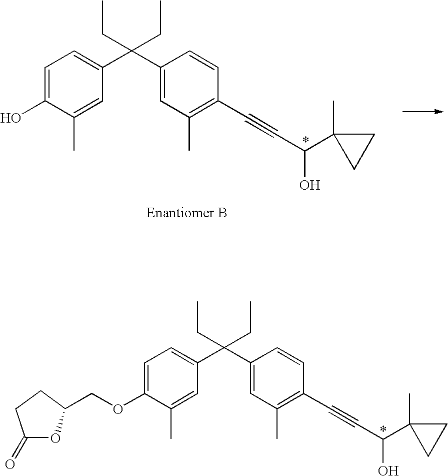

To a solution of compound prepared in Example 267-(2) (4-(1-ethyl-1-{4-[3-hydroxy-3-(1-methyl-cyclopropyl)-prop-1-ynyl]-3-methyl-phenyl}-propyl)-2-methyl-phenol (Enantiomer B) ) (8 mg, 0.0213 mmol) in DMF (0.5 ml), K₂CO3 (10 mg, 0.0725 mmol) and toluene-4-sulfonic acid (R)-5-oxo-tetrahydro-furan-2-ylmethyl ester (10 mg, 0.0756 mmol) were added at room temperature and the mixture was stirred at 90 degrees C. for 64 h. To the mixture, ethyl acetate was added and the mixture was washed with H₂O, dried over MgSO₄, concentrated in vacuo and the obtained residue was purified by preparative TLC (n-hexane:ethyl acetate=1:1 and CH₂Cl₂:MeOH=20:1) to give the title compound (4.9 mg, 49%).

¹H-NMR: 0.41 (q, 2H), 0.59 (t, 6H), 0.61-0.69 (m, 2H), 1.26 (s, 3H), 1.82 (d, 1H), 2.02 (q, 4H), 2.14 (s, 3H), 2.37 (s, 3H), 2.38-2.83 (m, 4H), 4.04-4.30 (m, 3H), 4.84-4.92 (m, 1H), 6.65 (d, 1 h), 6:84-6.72 (m, 4H), 7.25 (d, 1H); MS (ESI+): 457 ([M−OH]⁺).

### (2) Preparation of (R)-5-[4-(1-Ethyl-1-{4-[3-hydroxy-3-(1-methyl-cyclopropyl)-prop-1-ynyl]-3-methyl-phenyl}-propyl)-2-methyl-phenoxy]-4-hydroxy-pentanoic acid

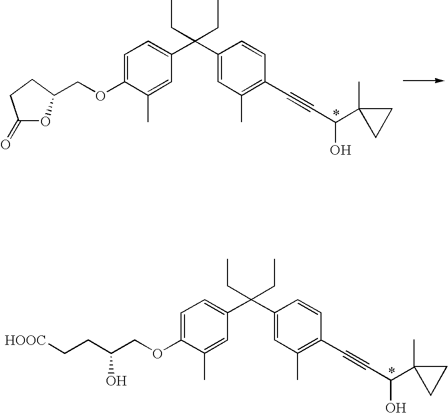

To a solution of (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one (prepared in Example 268-(1)) (4.9 mg, 0.010 mmol) in MeOH (0.5 ml) and THF (0.5 ml), 1NNaOH (0.1 ml) was added at room temperature and the mixture was stirred at room temperature for 1 h. The mixture concentrated in vacuo, and purified by preparative TLC (CHCl3:MeOH=8:3 saturated with H₂O) to give the title compound (1.3 mg, 25%).

¹H-NMR: 0.41 (q, 4H), 0.59 (t, 6H), 0.64-0.69 (m, 2H), 1.27 (s, 3H), 1.90-2.05 (m, 7H), 2.16 (s, 3H), 2.37 (s, 3H), 2.62 (t, 2H), 3.814.10 (m, 3H), 4.26 (s, 1H), 6.66 (s, 1H), 6.85-7.00 (m, 3H), 7.27 (s, 1H); MS (ESI−): 491 ([M−H]⁻).

### Example 269

### Preparation of (R)-5-[4-(1-Ethyl-1-{4-[3-hydroxy-3-(1-methyl-cyclopropyl)-propyl]-3-methyl-phenyl}-propyl)-2-methyl-phenoxy]-4-hydroxy-pentanoic acid

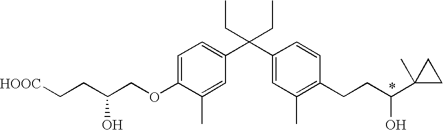

### (1) Preparation of 4-(1-Ethyl-1-{4-[3-hydroxy-3-(1-methyl-cyclopropyl)-propyl]-3-methyl-phenyl}-propyl)-2-methyl-phenol

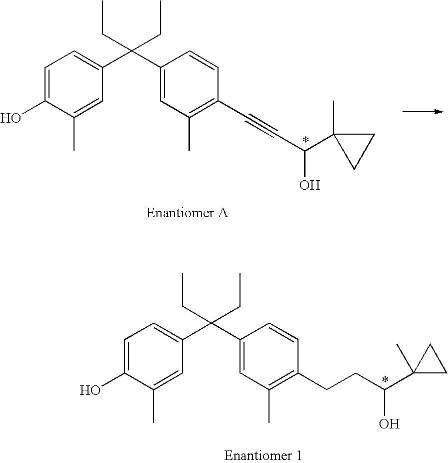

To a solution of compound prepared in Example 267-(2) (4-(1-ethyl-1-{4-[3-hydroxy-3-(1-methyl-cyclopropyl)-prop-1-ynyl]-3-methyl-phenyl}-propyl)-2-methyl-phenol (Enantiomer A) ) (15 mg, 0.0399 mmol) in ethyl acetate (2 ml), palladium carbon (5 mg) was added at room temperature and the mixture was stirred at room temperature for 3 h under hydrogen gas. The mixture was filtrated through silica and concentrated in vacuo to give the title compound (15 mg, 100%).

¹H-NMR: 0.30-0.40 (m, 4H), 0.59 (t, 3H), 1.05 (s, 3H), 1.76-1.84 (m, 2H), 2.03 (q, 4H), 2.19 (s, 3H), 2.25 (s, 3H), 2.53-2.80 (m, 2H), 2.89 (t, 1H), 4.68 (s, 1H), 6.64 (d, 1H), 6.82-7.04 (m, 5H); MS (ESI−): 379 ([M−H]⁻).

### (2) Preparation of (R)-5-[4-(1-Ethyl-1-{4-[3-hydroxy-3-(1-methyl-cyclopropyl)-propyl]-3-methyl-phenyl}-propyl )-2-methyl-phenoxymethyl]-dihydro-furan-2-one

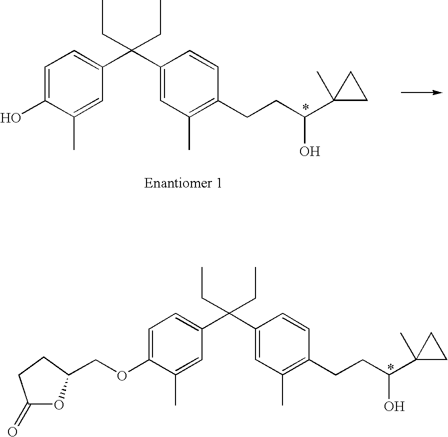

To a solution of 4-(1-Ethyl-1-{4-[3-hydroxy-3-(1-methyl-cyclopropyl)-propyl]-3-methyl-phenyl}-propyl)-2-methyl-phenol (Enantiomer 1 prepared in Example 269-(1)) (15 mg, 0.0395 mmol) in DMF (0.5 ml), K₂CO3 (10 mg, 0.0725 mmol) and toluene-4-sulfonic acid (R)-5-oxo-tetrahydro-furan-2-ylmethyl ester (10 mg, 0.0756 mmol) were added at room temperature and the mixture was stirred at 90 degrees C. for 64 h. To the mixture, ethyl acetate was added and the mixture was washed with H₂O, dried over MgSO₄, concentrated in vacuo and the obtained residue was purified by preparative TLC (n-hexane:ethyl acetate=1:1 and CH₂Cl₂:MeOH=20:1) to give the title compound (5.2 mg, 28%).

¹H-NMR: 0.28-0.42 (m, 4H), 0.59 (t, 6H), 1.05 (s, 3H), 1.26 (t, 1H), 1.78-1.84 (m, 2H), 2.02 (q, 4H), 2.17 (s, 3H), 2.25 (s, 3H), 2.29-2.92 (m, 6H), 4.01-4.20 (m, 2H), 4.82-4.92 (m, 1H), 6.66 (d, 1H), 6.89-7.04 (m, 5H); MS (ESI+): 496 ([M+NH4]⁺).

### (3) Preparation of (R)-5-[4-(1-Ethyl-1-{4-[3-hydroxy-3-(1-methyl-cyclopropyl)-propyl]-3-methyl-phenyl}-propyl)-2-methyl-phenoxy]4-hydroxy-pentanoic acid

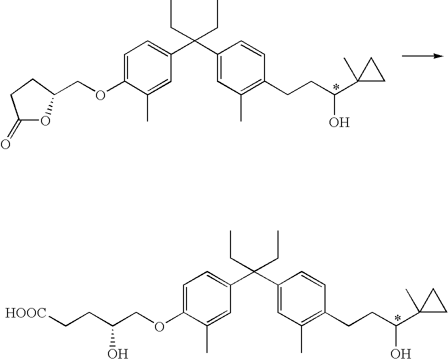

To a solution of ((R)-5-[4-(1-Ethyl-1-{4-[3-hydroxy-3-(1-methyl-cyclopropyl)-propyl]-3-methyl-phenyl}-propyl)-2-methyl-phenoxymethyl]-dihydro-furan-2-one) (prepared in Example 269-(2))(5.2 mg, 0.011 mmol) in MeOH (0.5 ml) and THF (0.5 ml), 1NNaOH (0.1 ml) was added at room temperature and the mixture was stirred at room temperature for 1 h. The mixture was concentrated in vacuo, and purified by preparative TLC (CHCl3:MeOH=8:3 saturated with H₂O) to give the title compound (3.9 mg, 73%).

¹H-NMR: 0.32-0.42 (m, 4H), 0.59 (t, 6H), 1.05 (s, 3H), 1.78-1.85 (m, 2H), 1.90-2.00 (m, 2H), 2.05 (q, 4H), 2.15 (s, 3H), 2.25 (s, 3H), 2.56-2.91 (m, 5H), 3.82-4.15 (m, 3H), 6.67 (d, 1H), 6.89-7.01 (m, 5H); MS (ESI−): 495 ([M−H]⁻).

### Example 270

### Preparation of (R)-5-[4-(1-Ethyl-1-{4-[3-hydroxy-3-(1-methyl-cyclopropyl)-propyl]-3-methyl-phenyl}-propyl)-2-methyl-phenoxy]-4-hydroxy-pentanoic acid

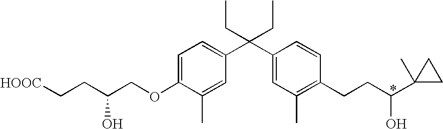

### Preparation of 4-(1-Ethyl-1-{4-[3-hydroxy-3-(1-methyl-cyclopropyl)-propyl]-3-methyl-phenyl}-propyl)-2-methyl-phenol

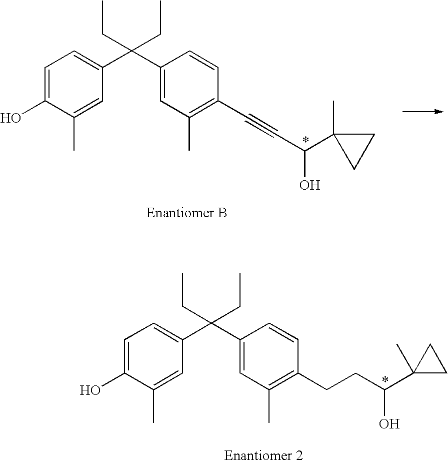

To a solution of compound prepared in Example 267-(2) (4-(1-ethyl-1-{4-[3-hydroxy-3-(1-methyl-cyclopropyl)-prop-1-ynyl]-3-methyl-phenyl}-propyl)-2-methyl-phenol (Enantiomer B)) (10 mg, 0.0266 mmol) in ethyl acetate (2 ml), palladium carbon (5 mg) was added at room temperature and the mixture was stirred at room temperature for 3 h under hydrogen gas. The mixture was filtrated through silica and concentrated in vacuo to give the title compound (10 mg, 100%).

¹H-NMR: 0.30-0.40 (m, 4H), 0.59 (t, 3H), 1.05 (s, 3H), 1.76-1.84 (m, 2H), 2.04 (q, 4H), 2.19 (s, 3H), 2.23 (s, 3H), 2.53-2.80 (m, 2H), 2.88 (t, 1H), 4.80 (s, 6.64 (d, 1H), 6.82-7.03 (m, 5H); MS (ESI−): 379 ([M−H]⁻).

### (2) Preparation of (R)-5-[4-(1-Ethyl-1-{4-[3-hydroxy-3-(1-methyl-cyclopropyl)-propyl]-3-methyl-phenyl}-propyl )-2-methyl-phenoxymethyl]-dihydro-furan-2-one

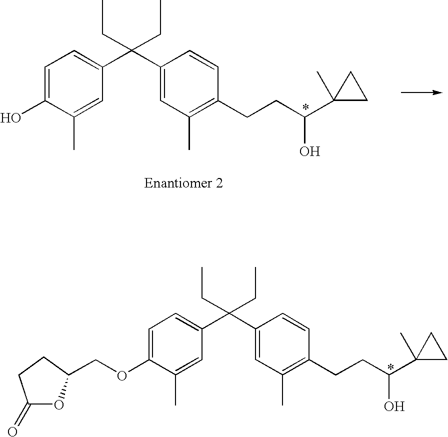

To a solution of 4-(1-Ethyl-1-{4-[3-hydroxy-3-(1-methyl-cyclopropyl)-propyl]-3-methyl-phenyl}-propyl)-2-methyl-phenol4-(1-Ethyl-1-{4-[3-hydroxy-3-(1-methyl-cyclopropyl)-propyl]-3-methyl-phenyl)-propyl)-2-methyl-phenol (Enantiomer 2 prepared in Example 270-(1)) (10 mg, 0.0263 mmol) in DMF (0.5 ml), K₂CO3 (10 mg, 0.0725 mmol) and toluene-4-sulfonic acid (R)-5-oxo-tetrahydro-furan-2-ylmethyl ester (10 mg, 0.0756 mmol) were added at room temperature and stirred at 90 degrees C. for 64 h. To the mixture, ethyl acetate was added and the organic layer was washed with H₂O, dried over MgSO₄, concentrated in vacuo and the obtained residue was purified by preparative TLC (n-hexane:ethyl acetate=1:1 and CH₂Cl₂:MeOH=20:1) to give the title compound (6.7 mg, 53%).

¹H-NMR: 0.28-0.42 (m, 4H), 0.59 (t, 6H), 1.05 (s, 3H), 1.26 (t, 1H), 1.78-1.84 (m, 2H), 2.04 (q, 4H), 2.15 (s, 3H), 2.25 (s, 3H), 2.25-2.95 (m, 6H), 4.01-4.22 (m, 2H), 4.83-4.94 (m, 1H), 6.66 (d, 1H), 6.89-7.04 (m, 5H); MS (ESI+): 496 ([M+NH4]⁺).

### (3) Preparation of (R)-5-[4-(1-Ethyl-1-{4-[3-hydroxy-3-(1-methyl-cyclopropyl)-propyl]-3-methyl-phenyl}-propyl)-2-methyl-phenoxy]-4-hydroxy-pentanoic acid

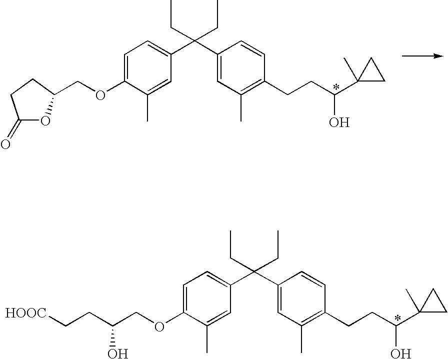

To a solution of (R)-5-[4-(1-Ethyl-1-{4-[3-hydroxy-3-(1-methyl-cyclopropyl)-propyl]-3-methyl-phenyl}-propyl )-2-methyl-phenoxymethyl]-dihydro-furan-2-one (prepared in Example 270-(2))(6.7 mg, 0.014 mmol) in MeOH (0.5 ml) and THF (0.5 ml), 1NNaOH (0.1 ml) was added at room temperature and the mixture was stirred at room temperature for 1 h. The mixture was concentrated in vacuo, and purified by preparative TLC (CHCl3:MeOH=8:3 saturated with H₂O) to give the title compound (3.6 mg, 52%).

¹H-NMR: 0.32-0.41 (m, 4H), 0.59 (t, 6H), 1.05 (s, 3H), 1.26 (s, 1H), 1.77-1.85 (m, 2H), 1.89-2.00 (m, 2H), 2.02 (q, 4H), 2.17 (s, 3H), 2.25 (s, 3H), 2.58-2.92 (m, 5H), 3.82-4.15 (m, 3H), 6.67 (d, 1H), 6.89-7.01 (m, 5H); MS (ESI−) :495 ([M−H]⁻]).

### Example 271

### Preparation of (R)-5-[4-(1-Ethyl-1-{4-[3-(1-ethyl-cyclopropyl)-3-hydroxy-propyl]-3-methyl-phenyl}-propyl)-2-methyl-phenoxy]-4-hydroxy-pentanoic acid

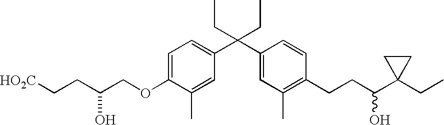

### (1) Preparation of Cyclopropanecarboxylic acid 2,6-di-tert-butyl-4-methyl-phenyl ester

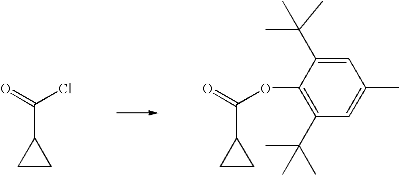

n-BuLi (2.44 mol/l in hexane) (22.7 mmol) was added to an ice-cooled solution of 2,6-Di-t-butyl-p-cresol (5.0 g, 22.7 mmol) in 25 ml of THF. After a period of 15 min, Cyclopropanecarbonyl Chloride (2.5 g, 23.8 mmol) was added and the mixture stirred at room temperature for 24 h and poured onto 10 ml of saturated aqueous NH₄Cl. After separation of the layers, the aqueous layer was extracted with 25 ml of ether, and the organic phase were combined and washed with saturated aqueous NaHCO₃ and brine, dried over MgSO₄, and concentrated in vacuo. The residue was chromatographed on silica gel (ethyl acetate/hexane=0/100 to 10/90) to give the title compound (5.6 g, 85%).

¹H-NMR: 1.00-1.07 (m, 2H), 1.13-1.18 (m, 2H), 1.34 (s, 18H), 1.89-1.97 (m, 1H), 2.31 (s, 3H), 7.10 (s, 2H); MS (ESI+): 306 ([M+NH4]⁺).

### (2) Preparation of 1-Ethyl-cyclopropanecarboxylic acid 2,6-di-tert-butyl-4-methyl-phenyl ester

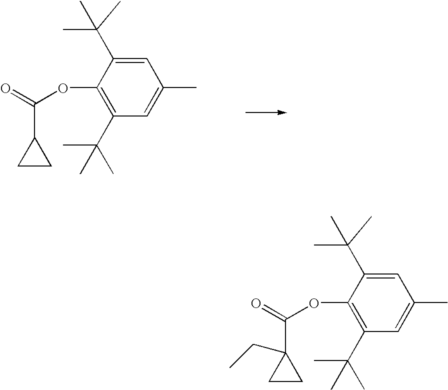

To a solution of Cyclopropanecarboxylic acid 2,6-di-tert-butyl-4-methyl-phenyl ester (compound prepared in Example 271-(1)) (3.0 g, 10.4 mmol) in THF (20 ml) was added t-BuLi (1.47 mol/l in n-pentane) (11.4 mmol) at −78 degrees C. The solution was stirred under N₂ for 30 min, and Ethyl Iodide (1.9 g, 12.5 mmol, dissolved in 3 ml of THF) was added. The solution was allowed to warm to room temperature within 4 h. The reaction was quenched with saturated aqueous NH₄Cl. The mixture was diluted with Et₂O, washed with saturated aqueous NH₄Cl and brine, dried over MgSO₄, and concentrated in vacuo to give the title compound (320 mg, 37%).

¹H-NMR: 0.89-0.93 (m, 2H), 1.05 (t, J=7.3 Hz, 3H), 1.32 (s, 18H), 1.45-1.52 (m, 2H), 1.82 (q, J=7.3 Hz, 2H), 2.30 (s, 3H), 7.09 (s, 2H)

### (3) Preparation of 1-Ethyl-cyclopropanecarboxylic acid

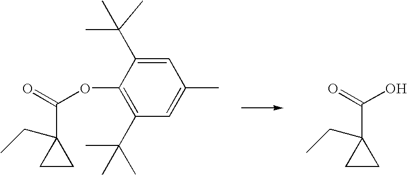

A suspension of 1-Ethyl-cyclopropanecarboxylic acid 2,6-di-tert-butyl-4-methyl-phenyl ester (compound prepared in Example 271-(2)) (2.41 g, 7.6 mmol), t-BuOK (5.13 g, 45.7 mmol), and H₂O (274 mg, 15.2 mmol) in 38 ml of THF was heated under reflux for 36h. After extraction with 2N KOH, the H₂O phase was acidified with concentrated HCl, extracted with Et₂O. The extracts were dried over MgSO₄ and concentrated in vacuo and the obtained residue was chromatographed on silica gel (Et₂O/n-pentane=3/97) to give the title compound (2.41 g, 73%).

¹H-NMR: 0.73-0.81 (m, 2H), 1.01 (t, J=7.3 Hz, 3H), 1.22-1.30 (m, 2H), 1.56 (q, J=7.3 Hz, 2H); MS (ESI+): 251 ([2×M+Na]⁺).

### (4) Preparation of 1-Ethyl-cyclopropanecarboxylic acid N-methoxy-N-methyl-amide

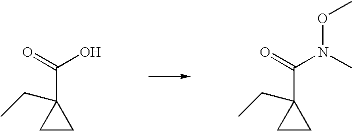

To a solution of 1-Ethyl-cyclopropanecarboxylic acid (compound prepared in Example 271-(3)) (320 mg, 2.8 mmol) in dichloromethane (10 ml) was added methoxymethylamine hydrochloride (328 mg, 3.4 mmol), 4-dimethylaminopyridine (171 mg, 1.4 mmol), triethyamine (793 mg, 7.8 mmol), and 1-ethyl-3-(3-dimethylaminopropyl)-carbodiimide hydrochloride (751 mg, 3.9 mmol) at 0 degrees C. The solution was stirred under N₂ for 20 h, and diluted with ethyl acetate, washed with 1N HCl, saturated aqueous NaHCO₃ and brine, dried over MgSO₄, and concentrated in vacuo. The residue was chromatographed on silica gel (ethyl acetate/hexane=0/100 to 40/60) to give the title compound (200 mg, 45%).

¹H-NMR: 0.55-0.57-(m, 2H), 0.93-1.02 (m, 5H), 1.59 (q, J=7.7 Hz, 2H), 3.25 (s, 3H), 3.72 (s, 3H); MS (ESI+): 158([M+H]⁺).

### (5) Preparation of 3-(4-{1-[4-(tert-Butyl-dimethyl-silanyloxy)-3-methyl-phenyl]-1-ethyl-propyl}-2-methyl-phenyl)-1-(1-ethyl-cyclopropyl)-prop-2-yn-1-one

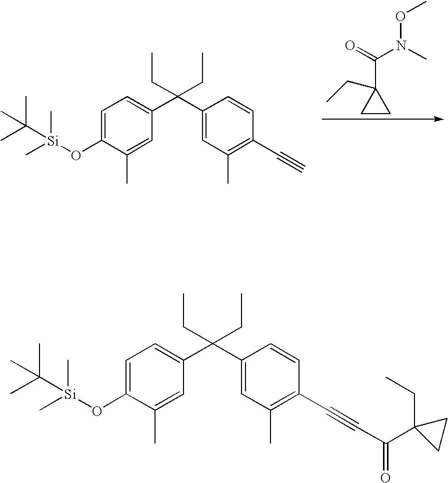

To a solution of tert-Butyl-{4-[1-ethyl-1-(4-ethynyl-3-methyl-phenyl)-propyl]-2-methyl-phenoxy}-dimethyl-silane (compound prepared in Example 260-(1)) (345 mg, 0.85 mmol) in THF (2 ml) was added n-BuLi (2.44 mol/l in hexane) (1.19 mmol) at −78 degrees C. The solution was stirred at room temperature under N₂ for 20 min. The solution was cooled to −78 degrees C., and added THF solution (0.5 ml) of 1-Ethyl-cyclopropanecarboxylic acid N-methoxy-N-methyl-amide (compound prepared in Example 271-(4)) (200 mg, 1.27 mmol). The solution was allowed to warm to room temperature overnight. The reaction was quenched with saturated aqueous NH₄Cl, extracted with ethyl acetate, washed with brine, dried over MgSO₄, and concentrated in vacuo. The residue was chromatographed on silica gel (ethyl acetate/hexane=0/100 to 5/95) to give the title compound (380 mg, 89%).

¹H-NMR: 0.20 (s, 6H), 0.59 (t, J=7.0 Hz, 6H), 0.95-1.06 (m, 14H), 1.51-1.56 (m, 2H), 1.73 (q, J=7.3 Hz, 2H), 2.03 (q, J=7.0 Hz, 4H), 2.14 (s, 3H), 2.41 (s, 3H), 6.63 (d, J=8.4 Hz, 1H), 6.79 (d, J=8.1 Hz, 1H), 6.84 (s, 1H), 6.99 (d, J=8.1 Hz, 1H), 7.04 (s, 1H), 7.38 (d, J=8.1 Hz, 1H); MS (ESI+): 503([M+H]⁺).

### (6) Preparation of 3-(4-{1-[4-(tert-Butyl-dimethyl-silanyloxy)-3-methyl-phenyl]-1-ethyl-propyl}-2-methyl-phenyl)-1-(1-ethyl-cyclopropyl)-prop-2-yn-1-ol

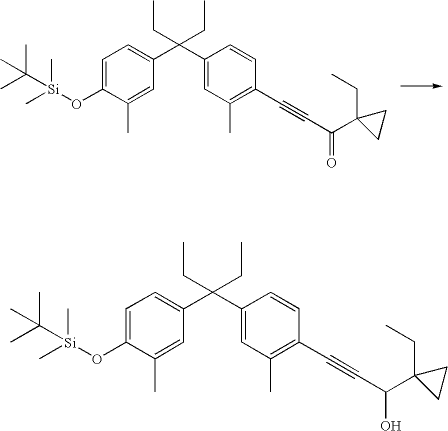

To a solution of 3-(4-{1-[4-(tert-Butyl-dimethyl-silanyloxy)-3-methyl-phenyl]-1-ethyl-propyl)-2-methyl-phenyl)-1-(1-ethyl-cyclopropyl)-prop-2-yn-1-one (compound prepared in Example 271-(5)) (380 mg, 0.76 mmol) in THF/MeOH (4 ml/4 ml) was added NaBH₄ (86 mg, 2.28 mmol) at room temperature. The solution was stirred at room temperature under N₂ for 1 h, quenched with H₂O, and extracted with ethyl acetate, washed with brine, dried over MgSO₄, and concentrated in vacuo. The residue was chromatographed on silica gel (ethyl acetate/hexane=0/100 to 10/90) to give the title compound (350 mg, 91%).

¹H-NMR: 0.19 (s, 6H), 0.35-0.46 (m, 2H), 0.58 (t, J=7.3 Hz, 6H), 0.68-0.78 (m, 2H), 0.95-1.03 (m, 12H), 1.30-1.40 (m, 1H), 1.74 (d, J=7.0 Hz, 1H), 1.87-1.96 (m, 1H), 2.03 (q, J=7.0 Hz, 4H), 2.14 (s, 3H), 2.36 (s, 3H), 4.63 (d, J=7.0 Hz, 1H), 6.62 (d, J=8.4 Hz, 1H), 6.79 (d, J=8.8 Hz, 1H), 6.85 (s, 1H), 6.92 (d, J=8.1 Hz, 1H), 6.99 (s, 1H), 7.25 (m, 1H); MS (ESI−): 503([M−H]⁻).

### (7) Preparation of 4-(1-Ethyl-1-{4-[3-(1-ethyl-cyclopropyl)-3-hydroxy-prop-1-ynyl]-3-methyl-phenyl}-propyl)-2-methyl-phenol

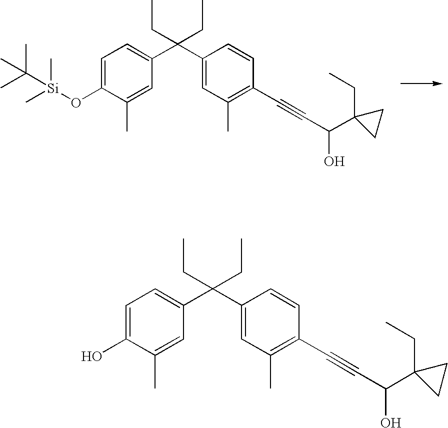

To a solution of 3-(4-{1-[4-(tert-Butyl-dimethyl-silanyloxy)-3-methyl-phenyl]-1-ethyl-propyl}-2-methyl-phenyl)-1-(1-ethyl-cyclopropyl)-prop-2-yn-1-ol (compound prepared in Example 271-(6)) (350 mg, 0.69 mmol) in THF (7 ml) was added tetrabutylammonium fluoride (1 M in THF) (1.0 mmol) at room temperature. The solution was stirred at room temperature under N₂ for 30 min, and diluted with ethyl acetate, washed with diluted KHSO₄ and brine, dried over MgSO₄, and concentrated in vacuo. The residue was chromatographed on silica gel (ethyl acetate/hexane=0/100 to 40/60) to give the title compound (240 mg, 89%).

¹H-NMR: 0.35-0.46 (m, 2H), 0.59 (t, J=7.3 Hz, 6H), 0.68-0.78 (m, 2H), 0.97 (t, J=7.7 Hz, 3H), 1.29-1.41 (m, 1H), 1.74 (d, J=7.0 Hz, 1H), 1.87-1.97 (m, 1H), 2.02 (q, J=7.3 Hz, 4H), 2.19 (s, 3H), 2.36 (s, 3H), 4.57 (d, J=4.4 Hz, 1H), 4.63 (d, J=6.6 Hz, 1H), 6.65 (d, J=8.1 Hz, 1H), 6.83-6.88 (m, 2H), 6.92 (d, J=8.1 Hz, 1H), 6.99 (s, 1H), 7.28-7.30 (m, 1H); MS (ESI−): 389([M−H]⁻).

### (8) Preparation of 4-(1-Ethyl-1-{4-[3-hydroxy-3-(1-ethyl-cyclopropyl)-propyl]-3-methyl-phenyl)-propyl)-2-methyl-phenol

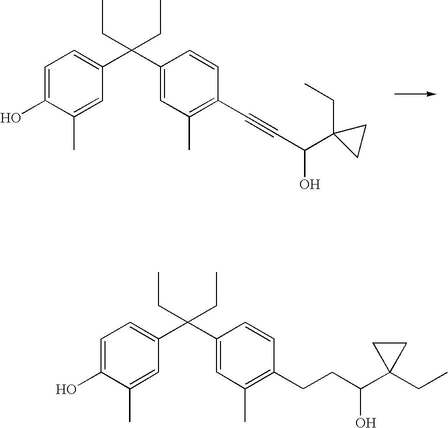

A solution of 4-(1-Ethyl-1-{4-[3-(1-ethyl-cyclopropyl)-3-hydroxy-prop-1-ynyl]-3-methyl-phenyl}-propyl)-2-methyl-phenol (compound prepared in Example 271-(7)) (38 mg, 0.097 mmol) in AcOEt/MeOH (1 ml/1 ml) was hydrogenated on 10% palladium on carbon (5 mg) as catalyst. After 4h the reaction mixture was filtered through a Celite, the filtrate was concentrated in vacuo. The residue was purified by preparative TLC (ethyl acetate/hexane=1/3) to give the title compound (33 mg, 87%).

¹H-NMR: 0.30-0.42 (m, 4H), 0.59 (t, J=7.3 Hz, 6H), 0.87 (t, J=7.3 Hz, 3H), 1.20-1.38 (m, 2H), 1.60-1.72 (m, 1H), 1.73-1.86 (m, 2H), 2.02 (q, J=7.7 Hz, 4H), 2.20 (s, 3H), 2.25 (s, 3H), 2.53-2.62 (m, 1H), 2.72-2.82 (m, 1H), 3.08-3.15 (m, 1H), 4.55 (s, 1H), 6.64 (d, J=8.1 Hz, 1H), 6.85-6.95 (m, 4H), 7.01 (d, J=8.4 Hz, 1H); MS (ESI−): 393([M−H]⁻).

### (9) Preparation of (R)-5-[4-(1-Ethyl-1-{4-[3-(1-ethyl-cyclopropyl)-3-hydroxy-propyl]-3-methyl-phenyl}-propyl)-2-methyl-phenoxymethyl]-dihydro-furan-2-one

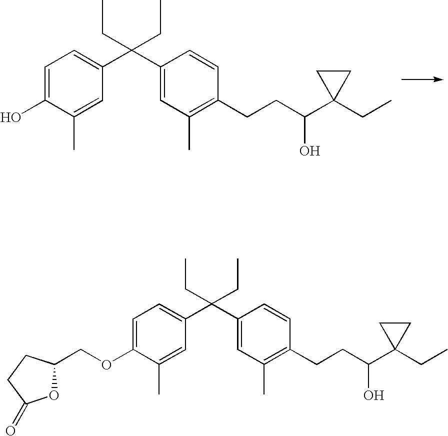

To a solution of 4-(1-Ethyl-1-{4-[3-hydroxy-3-(1-ethyl-cyclopropyl)-propyl]-3-methyl-phenyl}-propyl)-2-methyl-phenol (compound prepared in Example 271-(8)) (33 mg, 0.084 mmol) in dimethylacetamide (0.8 ml), K₂CO₃ (35 mg, 0.252 mmol) was added and stirred at room temperature. To the mixture, (R)-(−)-dihydro-5-(p-tolylsulfonyloxymethyl)-2-(3H)-furanone (34 mg, 0.126 mmol) was added and stirred at 100 degrees C. for 15 h. After cooling to room temperature, the reaction mixture was diluted with ethyl acetate, washed with H₂O and brine, dried over MgSO₄, and concentrated in vacuo. The residue was purified by preparative TLC (ethyl acetate/hexane=2/3) to give the title compound (24 mg, 58%).

¹H-NMR: 0.31-0.42 (m, 4H), 0.59 (t, J=7.3 Hz, 6H), 0.86 (t, J=7.3 Hz, 3H), 1.20-1.40 (m, 2H), 1.60-1.72 (m, 1H), 1.73-1.82 (m, 2H), 2.03 (q, J=7.3 Hz, 4H), 2.15 (s, 3H), 2.25 (s, 3H), 2.28-2.50 (m, 2H), 2.52-2.62 (m, 2H), 2.72-2.82 (m, 2H), 3.08-3.15 (m, 1H), 4.02-4.20 (m, 2H), 4.85-4.92 (m, 1H), 6.65 (d, J=8.4 Hz, 1H), 6.85-6.97 (m, 4H), 7.01 (d, J=8.4 Hz, 1H); MS (ESI−): 491 ([M−H]⁻).

### (10) Preparation of (R)-5-[4-(1-Ethyl-1-{4-[3-(1-ethyl-cyclopropyl)-3-hydroxy-propyl]-3-methyl-phenyl}-propyl)-2-methyl-phenoxy]-4-hydroxy-pentanoic acid

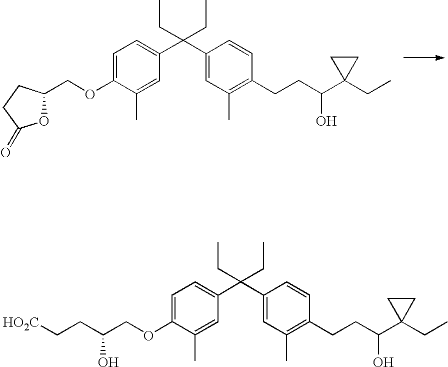

To a solution of (R)-5-[4-(1-Ethyl-1-{4-[3-(1-ethyl-cyclopropyl)-3-hydroxy-propyl]-3-methyl-phenyl}-propyl )-2-methyl-phenoxymethyl]-dihydro-furan-2-one (compound prepared in Example 271-(9)) (21 mg, 0.084 mmol) in THF/MeOH (2.5 ml/2.5 ml ), 1N NaOH (0.5 ml) was added at room temperature and stirred at room temperature for 1 h. The mixture was concentrated in vacuo and purified by preparative TLC (CHCl₃:MeOH=8:3 saturated with H₂O) to give the title compound (21 mg, 96%).

¹H-NMR: 0.30-0.42 (m, 4H), 0.59 (t, J=7.0 Hz, 6H), 0.86 (t, J=7.3 Hz, 3H), 1.23-1.37 (m, 1H), 1.60-1.72 (m, 1H), 1.73-2.20 (m, 9H), 2.16 (s, 3H), 2.25 (s, 3H), 2.53-2.65 (m, 3H), 2.72-2.80 (m, 1H), 3.08-3.15 (m, 1H), 3.80-3.98 (m, 2H), 4.00-4.09 (m, 1H), 6.66 (d, J=8.4 Hz, 1H), 6.85-6.97 (m, 4H), 7.01 (d, J=8.1 Hz, 1H); MS (ESI−): 509([M−H]⁻).

### Example 272

### Preparation of (R)-5-(4-{1-Ethyl-1-[3-methyl-4-(5,5,5-trifluoro-3-hydroxy-4-methyl-4-trifluoromethyl-pentyl)-phenyl]-propyl}-2-methyl-phenoxy)-4-hydroxy-pentanoic acid

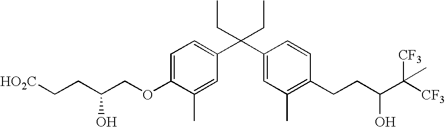

### (1) Preparation of 1-(4-{1-[4-(tert-Butyl-dimethyl-silanyloxy)-3-methyl-phenyl]-1-ethyl-propyl}-2-methyl-phenyl)-5,5,5-trifluoro-4-methyl-4-trifluoromethyl-pent-1-yn-3-one

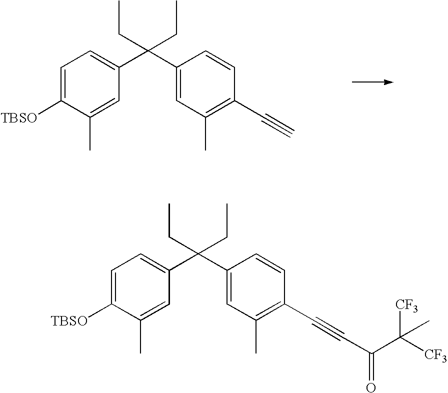

To a solution of tert-Butyl-{4-[1-ethyl-1-(4-ethynyl-3-methyl-phenyl)-propyl]-2-methyl-phenoxy}-dimethyl-silane (compound prepared in Example 260-(1)) (296.3 mg, 0.729 mmol) in THF (1.2 ml), 2.44 M n-butyl lithium in hexane (0.420 ml, 1.02 mmol) was added at −78 degrees C. To the mixture, 3,3,3-Trifluoro-2-methyl-2-trifluoromethyl-propionyl fluoride (0.160 ml, 1.11 mmol) in THF (0.5 ml) was added at −78 degrees C., the mixture was stirred at −78 degrees C. to room temperature overnight. To the mixture, brine was added. The products were extracted with ethyl acetate and the extracts were dried over MgSO₄, concentrated in vacuo. The obtained residue was chromatographed on silica gel (ethyl acetate/hexane=0/100 to 3/97) to give the title compound (289.8 mg, 66%).

¹H-NMR (chloroform-d): 0.20 (s, 6H), 0.59 (t, 6H, J=7.1 Hz), 1.00 (s, 9H), 1.77 (s, 3H), 2.04 (q, 4H, J=7.2 Hz), 2.14 (s, 3H), 2.45 (s, 3H), 6.64 (d, 1H, J=8.4 Hz), 6.79 (d, 1H, J=8.8 Hz), 6.84 (s, 1H), 7.04 (d, 1H, J=7.7 Hz), 7.09 (s, 1H), 7.47 (d, 1H, J=8.4 Hz); MS (ESI+): 599 ([M+H]⁺).

### (2) Preparation of 1-(4-{1-[4-(tert-Butyl-dimethyl-silanyloxy)-3-methyl-phenyl]-1-ethyl-propyl}-2-methyl-phenyl)-5,5,5-trifluoro-4-methyl-4-trifluoromethyl-pent-1-yn-3-ol

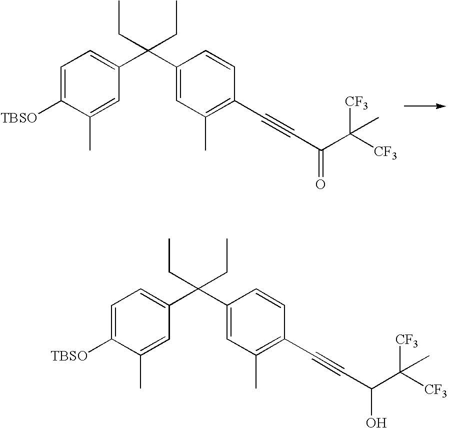

To a solution of 1-(4-{1-[4-(tert-Butyl-dimethyl-silanyloxy)-3-methyl-phenyl]-1-ethyl-propyl}-2-methyl-phenyl)-5,5,5-trifluoro-4-methyl-4-trifluoromethyl-pent-1-yn-3-one (compound prepared in Example 272-(1)) (289 mg, 0.483 mmol) in MeOH (1 ml) and THF (4 ml), NaBH₄ was added at room temperature, and the mixture was stirred at room temperature for 30 min. To the mixture, ethyl acetate was added and the solution was washed with brine, dried over MgSO₄, concentrated in vacuo. The obtained residue was chromatographed on silica gel (ethyl acetate/hexane=0/100 to 5/95) to give the title compound (209.9 mg, 72 %).

¹H-NMR (chloroform-d): 0.19 (s, 6H), 0.58 (t, 6H, J=7.1 Hz), 1.00 (s, 9H), 1.54 (s, 3H), 2.02 (q, 4H, J=7.1 Hz), 2.14 (s, 3H), 2.28 (d, 1H, J=8.1 Hz), 2.36 (s, 3H), 0.00 (d, 1H, J=7.7 Hz), 6.63 (d, 1H, J=8.4 Hz), 6.80 (d, 1H, J=8.4 Hz), 6.85 (s, 1H), 6.96 (d, 1H, J=8.0 Hz), 7.02 (s, 1H), 7.28 (d, 1H, J=8.4 Hz); MS(ESI+): 601 ([M+H]⁺).

### (3) Preparation of Acetic acid 3-(4-{1-[4-(tert-butyl-dimethyl-silanyloxy)-3-methyl-phenyl]-1-ethyl-propyl}-2-methyl-phenyl)-1-(2,2,2-trifluoro-1-methyl-1-trifluoromethyl-ethyl)-prop-2-ynyl ester

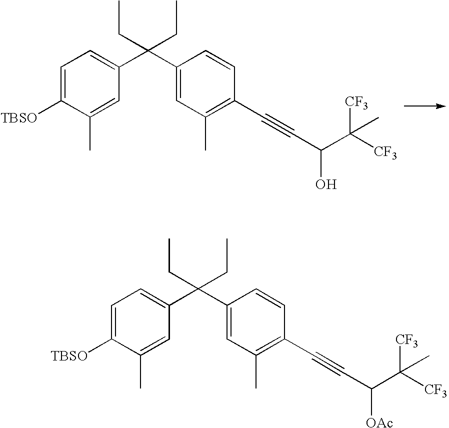

To a solution of 1-(4-{1-[4-(tert-Butyl-dimethyl-silanyloxy)-3-methyl-phenyl]-1-ethyl-propyl-2-methyl-phenyl)-5,5,5-trifluoro-4-methyl-4-trifluoromethyl-pent-1-yn-3-ol (compound prepared in Example 272-(2)) (23.8 mg, 0.0396 mmol) in CH₂Cl₂ (1 ml), pyridine (0.0096 ml) and Ac₂O (0.0056 ml) were added at room temperature, and the mixture was stirred at room temperature for 12 h. CH₂Cl₂ was removed in vacuo and pyridine (0.3 ml) and Ac₂O (0.0112 ml) were added to the mixture again and the mixture was stirred at room temperature for 1.5 h.

To the mixture ethyl acetate was added and the solution was washed with dil.HCl and brine, dried over MgSO₄, concentrated in vacuo. The obtained residue was purified by preparative TLC (ethyl acetate/hexane=1/20) to give the title compound (21.3 mg, 84%).

¹H-NMR (chloroform-d): 0.19 (s, 6H), 0.58 (t, 6H, J=7.1 Hz), 1.00 (s, 9H), 1.59 (s, 3H), 2.02 (q, 4H, J=7.2 Hz), 2.14 (s, 3H), 2.14 (s, 3H), 2.34 (s, 3H), 6.11 (s, 1H), 6.62 (d, 1H, J=8.8 Hz), 6.78 (d, 1H, J=8.1 Hz), 6.84 (s, 1H), 6.94 (d, 1H, J=8.8 Hz), 7.00 (s, 1H), 7.28 (d, 1H, J=8.1 Hz); MS(ESI+): 665 ([M+Na]⁺).

### (4) Preparation of Acetic acid 3-{4-[1-ethyl-1-(4-hydroxy-3-methyl-phenyl)-propyl]-2-methyl-phenyl}-1-(2,2,2-trifluoro-1-methyl-1-trifluoromethyl-ethyl)-prop-2-ynyl ester

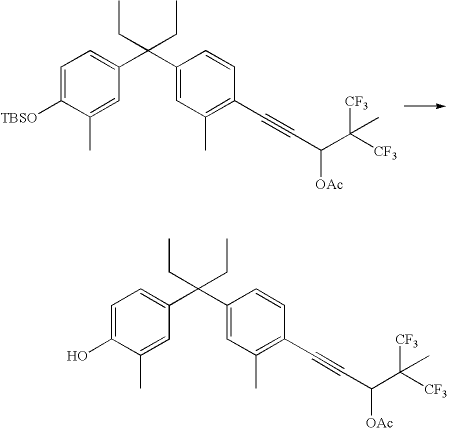

To a solution of Acetic acid 3-(4-{1-[4-(tert-butyl-dimethyl-silanyloxy)-3-methyl-phenyl]-1-ethyl-propyl}-2-methyl-phenyl)-1-(2,2,2-trifluoro-1-methyl-1-trifluoromethyl-ethyl)-prop-2-ynyl ester (compound prepared in Example 272-(3)) (19.9 mg, 0.0310 mmol) in THF (2 ml), 1 M terabutylammonium fluoride in THF (0.040 ml, 0.040 mmol) was added. The mixture was stirred for 5 min. To the mixture, ethyl acetate was added and the solution was washed with brine, dried over MgSO₄, concentrated in vacuo, The obtained residue was purified by preparative TLC (ethyl acetate/hexane=1/10) to give the title compound (14.1 mg, 86%).

¹H-NMR (chloroform-d): 0.58 (t, 6H, J=7.3 Hz), 1.59 (s, 3H), 2.02 (q, 4H, J=7.0 Hz), 2.14 (s, 3H), 2.18 (s, 3H), 2.34 (s, 3H), 4.85 (brs, 1H), 6.11 (s, 1H), 6.65 (d, 1H, J=8.0 Hz), 6.82 (d, 1H, J=8.1 Hz), 6.83 (s, 1H), 6.95 (d, 1H, J=8.4 Hz), 7.00 (s, 1H, J=8.4 Hz); MS(ESI−): 527 ([M−H]⁻).

### (5) Preparation of Acetic acid 1-(2-{4-[1-ethyl-1-(4-hydroxy-3-methyl-phenyl)-propyl]-2-methyl-phenyl)-ethyl)-2,2-dimethyl-propyl ester

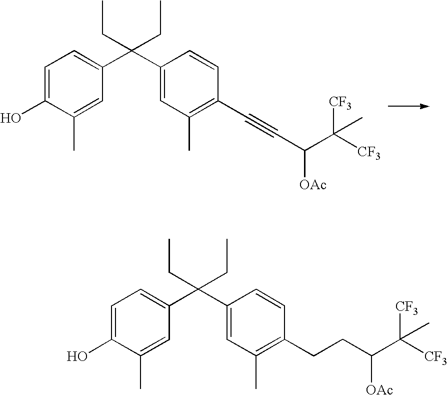

To a solution of Acetic acid 3-{4-[1-ethyl-1-(4-hydroxy-3-methyl-phenyl)-propyl]-2-methyl-phenyl}-1-(2,2,2-trifluoro-1-methyl-1-trifluoromethyl-ethyl)-prop-2-ynyl ester (compound prepared in Example 272-(4)) (14.1 mg, 0.0267 mmol) in MeOH (2 ml), 10%Pd/C (4.7 mg) was added. The mixture was stirred at room temperature for 13 h under hydrogen. The mixture was filtered, concentrated in vacuo and purified by preparative TLC (ethyl acetate/hexane=1/5) to give the title compound (12.9 mg, 91%).

¹H-NMR (chloroform-d): 0.59 (t, 6H, J=7.3 Hz), 1.37 (s, 3H), 1.90-2.05 (m, 6H), 2.12 (s, 3H), 2.19 (s, 3H), 2.20 (s, 3H), 2.45-2.58 (m, 2H), 4.69 (brs, 1H), 5.47 (d, 1H), 6.65 (d, 1H), 6.84-6.99 (m, 5H); MS(ESI−): 531 ([M−H]⁻).

### (6) Preparation of Acetic acid 1-[2-(4-{1-ethyl-1-[3-methyl-4-((R)-5-oxo-tetrahydro-furan-2-ylmethoxy)-phenyl]-propyl}-2-methyl-phenyl)-ethyl]-3,3,3-trifluoro-2-methyl-2-trifluoromethyl-propyl ester

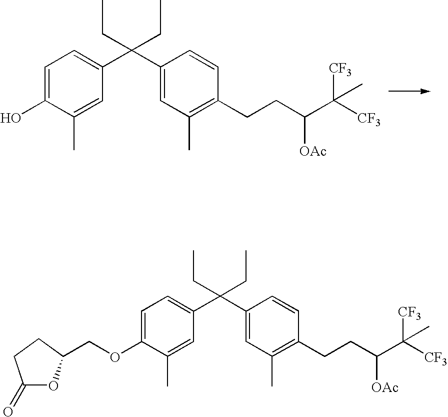

To a solution of Acetic acid 1-(2-{4-[1-ethyl-1-(4-hydroxy-3-methyl-phenyl)-propyl]-2-methyl-phenyl}-ethyl)-2,2-dimethyl-propyl ester (compound prepared in Example 272-(5)) (12.9 mg, 0.0242 mmol) in N,N-dimethylacetamide (0.5 ml), K₂CO₃ (11.1 mg, 0.0803 mmol) and (R)-(−)-dihydro-5-(p-tolylsulfonyloxymethyl)-2(3H)-furanone (10.2 mg, 0.0377 mmol) were added. The mixture was stirred at 90 degrees C. for 27 h. After cooled to room temperature, (R)-(−)-dihydro-5-(p-tolylsulfonyloxymethyl)-2(3H)-furanone (10.2 mg, 0.0377 mmol) was added. The mixture was stirred at 90 degrees C. for 14 h. After cooled to room temperature, the mixture was poured into brine and the products were extracted with ethyl acetate. The organic layer was washed with brine, dried over MgSO₄, concentrated in vacuo. The obtained residue was purified by preparative TLC (ethyl acetate/hexane=1/1) to give the title compound (11.2 mg, 73%).

¹H-NMR (chloroform-d): 0.58 (t, 6H, J=7.1 Hz), 1.37 (s, 3H), 1.92-2.06 (m, 6H), 2.12 (s, 3H), 2.15 (s, 3H), 2.20 (s, 3H), 2.24-2.36 (m, 1H), 2.39-2.61 (m, 4H), 2.73-2.82 (m, 1H), 4.06 (dd, 1H, J=3.4, 10.2 Hz), 4.16 (dd, 1H, J=3.2, 10.4 Hz), 4.87-4.90 (m, 1H), 5.46 (d, 1H), 6.66 (d, 1H), 6.90-7.00 (m, 5H); MS(ESI+): 631 ([M+H]⁺).

### (7) Preparation of (R)-5-(4-{1-Ethyl-1-[3-methyl-4-(5,5,5-trifluoro-3-hydroxy-4-methyl-4-trifluoromethyl-pentyl)-phenyl]-propyl}-2-methyl-phenoxy)-4-hydroxy-pentanoic acid

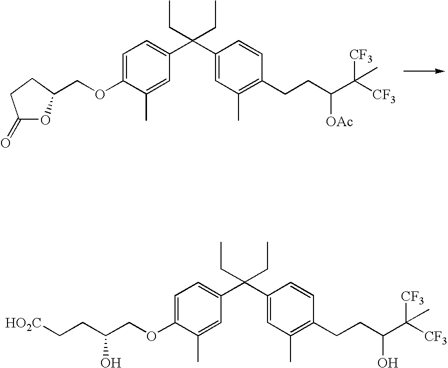

To a solution of Acetic acid 1-[2-(4-{1-ethyl-1-[3-methyl-4-((R)-5-oxo-tetrahydro-furan-2-ylmethoxy)-phenyl]-propyl}-2-methyl-phenyl)-ethyl]-3,3,3-trifluoro-2-methyl-2-trifluoromethyl-propyl ester (compound prepared in Example 272-(6)) (6.8 mg, 0.0108 mmol) in MeOH (0.5 ml) and THF (0.5 ml), 1N NaOH solution was added. The mixture was stirred at room temperature for 5.5 h. The mixture was concentrated in vacuo and purified by preparative TLC (CHCl₃/MeOH=8/3 saturated with H₂O) to give the title compound (4.7 mg, 72%).

¹H-NMR (chloroform-d): 0.59 (t, 6H, J=7.3 Hz), 1.35 (s, 3H), 1.74-2.06 (m, 8H), 2.16 (s, 3H), 2.25 (s, 3H), 2.57-2.63 (m, 3H), 2.91-2.98 (m, 1H), 3.83-3.90 (m, 2H), 3.97 (d, 1H, J=9.2 Hz), 4.04-4.11 (m, 1H), 6.67 (d, 1H, J=8.5 Hz), 6.91-6.96 (m, 4H), 7.00 (d, 1H, J=8.0 Hz); MS(ESI+); 607([M+H]⁺).

### Example 273

### Preparation of 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid

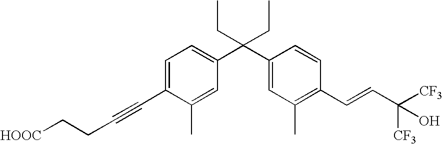

### (1) Preparation of Trifluoro-methanesulfonic acid 4-{1-ethyl-1-[3-methyl-4-(4,4,4-trifluoro-3-methoxymethoxy-3-trifluoromethyl-(E)-but-1-enyl)-phenyl]-propyl}-2-methyl-phenyl ester

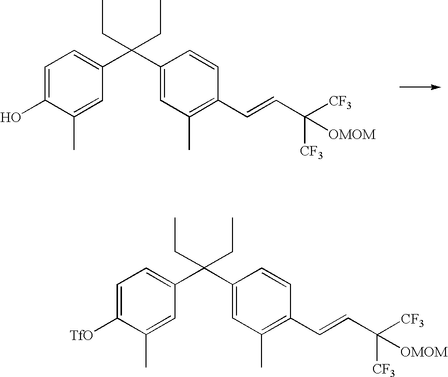

To a solution of 4-{1-Ethyl-1-[3-methyl-4-(4,4,4-trifluoro-3-methoxymethoxy-3-trifluoromethyl-(E)-but-1-enyl)-phenyl]-propyl}-2-methyl-phenol (compound prepared in Example 30-(1)) (0.2 g, 0.4 mmol) in dichloromethane (4 ml), pyridine (0.07 ml, 0.87 mmol), and Tf₂O (0.08 ml, 0.48 mmol) were added at 0 degrees C. and the mixture was stirred at room temperature for 15 minutes. To the mixture, ethyl acetate was added and the mixture was washed with water, dried over MgSO₄, concentrated in vacuo, and the obtained residue was chromatographed on silica gel (ethyl acetate/hexane=1/20) to give the title compound (0.22 g, 87%) as colorless oil.

¹H-NMR (CDCl₃): 0.61 (t, 6H, J=7.3 Hz), 2.08 (q, 4H, J=7.3 Hz), 2.32 (s, 3H), (s, 3H), 2.50 (s, 3H), 4.97 (s, 2H), 6.08 (d, 1H, J=16.6 Hz), 6.95-7.15 (m, 5H), 7.30-7.40 (m, 2H).

### (2) Preparation of Trifluoro-methanesulfonic acid 4-{1-ethyl-1-[3-methyl-4-(4,4,4-trifluoro-3-hydroxy-3-trifluoromethyl-(E)-but-1-enyl)-phenyl]-propyl}-2-methyl-phenyl ester

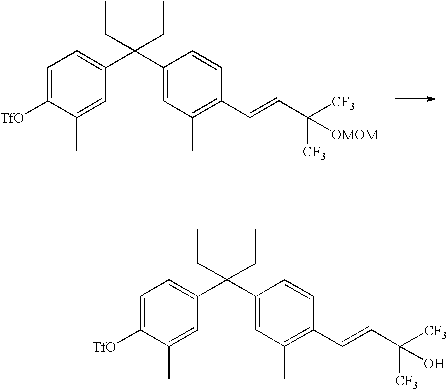

To a solution of Trifluoro-methanesulfonic acid 4-{1-ethyl-1-[3-methyl-4-(4,4,4-trifluoro-3-methoxymethoxy-3-trifluoromethyl-(E)-but-1-enyl)-phenyl]-propyl}-2-methyl-phenyl ester (compound prepared in Example 273-(1) (220 mg, 0.35 mmol) in dichloromethane (0.75 ml), trifluoroacetic acid (0.25 ml) was added at room temperature and the mixture was stirred at room temperature for 14 h. The mixture was concentrated in vacuo, and chromatographed on silica gel (ethyl acetate/hexane=1/0) to give the title compound (180 mg, 88%) as colorless oil.

¹H-NMR (CDCl₃): 0.61 (t, 6H, J=7.3 Hz), 2.08 (q, 4H, J=7.3 Hz), 2.32 (s, 3H), (s, 3H), 3.43 (s, 1H), 6.09 (d, 1H, J=15.8 Hz), 6.90-7.15 (m, 5H), 7.30-7.45 (m, 2H).

### (3) Preparation of 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid methyl ester

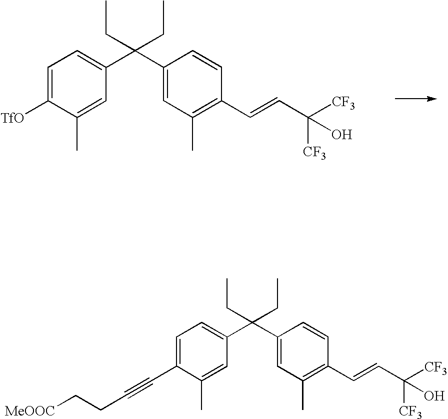

To a solution of trifluoro-methanesulfonic acid 4-{1-ethyl-1-[3-methyl-4-(4,4,4-trifluoro-3-hydroxy-3-trifluoromethyl-(E)-but-1-enyl)-phenyl]-propyl}-2-methyl-phenyl ester (compound prepared in Example 273-(2)) (30 mg, 0.05 mmol) in DMF (1 ml), PdCl₂(dppf)₂-CH₂Cl₂ (4 mg, 0.005 mmol), Et₃N (0.024 ml, 0.17 mmol), Cul (10 mg, 0.05 mmol), and pent-4-ynoic acid methyl ester (11 mg, 0.1 mmol) were added at room temperature under nitrogen and stirred at 120 degrees C. for 14 h. To the mixture, ethyl acetate was added and the mixture was washed with water and brine, dried over MgSO₄, concentrated in vacuo, and the obtained residue was chromatographed on silica gel (ethyl acetate/hexane=1/5) to give the title compound (20 mg, 71%) as colorless oil.

¹H-NMR (CDCl₃): 0.60 (t, 6H, J=7.3 Hz), 2.06 (q, 4H, J=7.3 Hz), 2.32 (s, 3H), 2.33 (s, 3H), 2.55-2.80 (m, 4H), 3.12 (s, 1H), 3.71 (s, 3H), 6.08 (d, 1H, J=15.8 Hz), 6.80-7.00 (m, 4H), 7.23 (d, 1H, J=8.1 Hz), 7.30-7.40 (m, 2H).

### (4) Preparation of 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid

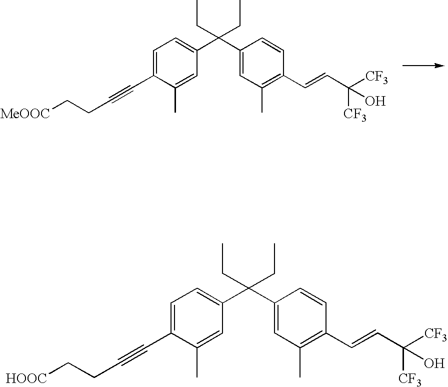

To a solution of 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid methyl ester (compound prepared in Example 273-(3)) (20 mg, 0.036 mmol) in MeOH (1 ml), 1N NaOH aq. (0.2 ml, 0.2 mmol) was added at room temperature and stirred at room temperature for 4 h. To the mixture, ethyl acetate was added and the mixture was washed with 30% NaH₂PO₄ aq., dried over MgSO₄, concentrated in vacuo, and the obtained residue was chromatographed on silica gel (ethyl acetate/hexane=3/1) to give the title compound (12 mg, 62%) as colorless oil.

¹H-NMR (CDCl₃): 0.60 (t, 6H, J=7.3 Hz), 2.06 (q, 4H, J=7.3 Hz), 2.32 (s, 3H), 2.34 (s, 3H), 2.60-2.85 (m, 4H), 6.08 (d, 1H, J=15.7 Hz), 6.80-7.00 (m, 4H), 7.23 (d, 1H, J=7.9 Hz), 7.30-7.40 (m, 2H); MS(ESI+): 541([M+H]⁺)

### Example 274

### Preparation of 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid

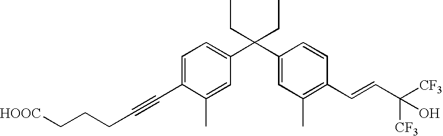

### (1) Preparation of 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid methyl ester

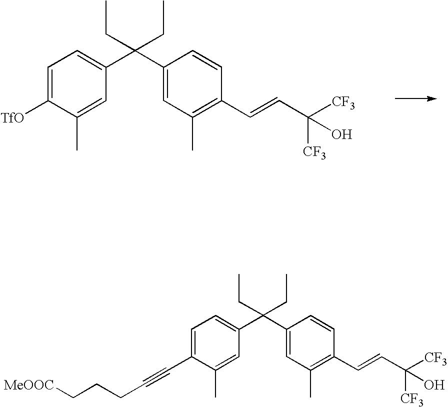

To a solution of trifluoro-methanesulfonic acid 4-{1-ethyl-1-[3-methyl-4-(4,4,4-trifluoro-3-hydroxy-3-trifluoromethyl-(E)-but-1-enyl)-phenyl]-propyl}-2-methyl-phenyl ester (compound prepared in Example 273-(2)) (30 mg, 0.05 mmol) in DMF (1 ml), PdCl₂(dppf)₂-CH₂Cl₂ (4 mg, 0.005 mmol), Et₃N (0.024 ml, 0.17 mmol), Cul (10 mg, 0.05 mmol), and hex-5-ynoic acid methyl ester (13 mg, 0.1 mmol) were added at room temperature under nitrogen and stirred at 120 degrees C. for 14 h. To the mixture, ethyl acetate was added and the mixture was washed with water and brine, dried over MgSO₄, concentrated in vacuo, and the obtained residue was chromatographed on silica gel (ethyl acetate/hexane=1/5) to give the title compound (20 mg, 69%) as colorless oil.

¹H-NMR (CDCl₃): 0.60 (t, 6H, J=7.3 Hz), 1.80-2.00 (m, 2H), 2.08 (q, 4H, J=7.2 Hz), 2.33 (s, 3H), 2.35 (s, 3H), 2.45-2.60 (m, 4H), 3.68 (s, 3H), 6.08 (d, 1H, J=15.7 Hz), 6.84-7.02 (m, 4H), 7.20-7.28 (m, 1H), 7.30-7.40 (m, 2H).

### (2) Preparation of 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid

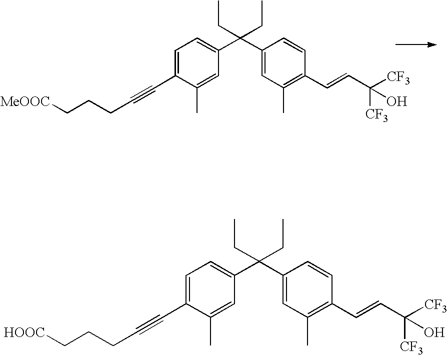

To a solution of 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid methyl ester (compound prepared in Example 274-(1)) (20 mg, 0.035 mmol) in MeOH (1 ml), 1N NaOH aq. (0.2 ml, 0.2 mmol) was added at room temperature and stirred at room temperature for 4 h. To the mixture, ethyl acetate was added and the mixture was washed with 30% NaH₂PO₄ aq., dried over MgSO₄, concentrated in vacuo, and the obtained residue was chromatographed on silica gel (ethyl acetate/hexane=3/1) to give the title compound (14 mg, 72%) as colorless oil.

¹H-NMR (CDCl₃): 0.60 (t, 6H, J=7.3 Hz), 1.85-2.00 (m, 2H), 2.06 (q, 4H, J=7.2 Hz), 2.33 (s, 3H), 2.35 (s, 3H), 2.54 (t, 2H, J=6.8 Hz), 2.58 (t, 2H, J=7.4 Hz), 6.11-6.05 (d, 1H, J=15.8 Hz), 6.85-7.05 (m, 4H), 7.20-7.27 (m, 1H), 7.30-7.40 (m, 2H); MS(ESI+): 555([M+H]⁺)

### Example 275

### Preparation of 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid

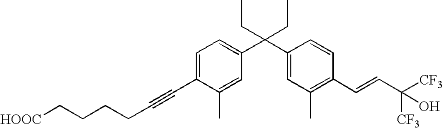

### (1) Preparation of 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid methyl ester

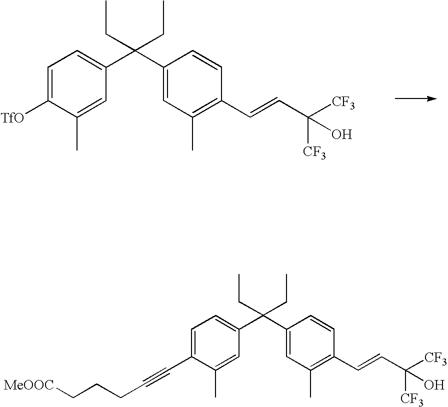

To a solution of trifluoro-methanesulfonic acid 4-{1-ethyl-1-[3-methyl-4-(4,4,4-trifluoro-3-hydroxy-3-trifluoromethyl-(E)-but-1-enyl)-phenyl]-propyl}-2-methyl-phenyl ester (compound prepared in Example 273-(2)) (30 mg, 0.05 mmol) in DMF (1 ml), PdCl₂(dppf)₂-CH₂Cl₂ (4 mg, 0.005 mmol), Et₃N (0.024 ml, 0.17 mmol), Cul (10 mg, 0.05 mmol), and hex-5-ynoic acid methyl ester (14 mg, 0.1 mmol) were added at room temperature under nitrogen and stirred at 120 degrees C. for 14 h. To the mixture, ethyl acetate was added and the mixture was washed with water and brine, dried over MgSO₄, concentrated in vacuo, and the obtained residue was chromatographed on silica gel (ethyl acetate/hexane=1/5) to give the title compound (18 mg, 61%) as colorless oil.

¹H-NMR (CDCl₃): 0.60 (t, 6H, J=7.3 Hz), 1.55-1.70 (m, 2H), 1.72-1.90 (m, 2H), 2.06 (q, 4H, J=7.3 Hz), 2.33 (s, 3H), 2.35 (s, 3H), 2.37 (t, 2H, J=7.1 Hz), 2.46 (t, 2H, J=6.9 Hz), 3.16 (s, 1H), 3.67 (s, 3H), 6.08 (d, 1H, J=16.1 Hz), 6.85-7.05 (m, 4H), 7.20-7.26 (m, 1H), 7.30-7.40 (m, 2H).

### (2) Preparation of 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid

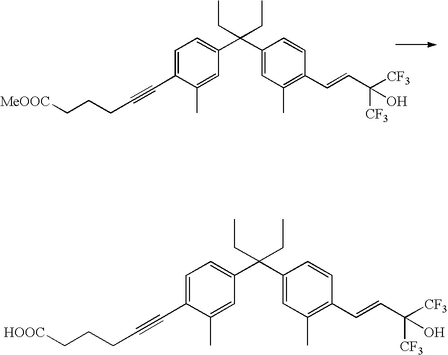

To a solution of 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid methyl ester (compound prepared in Example 275-(1)) (18 mg, 0.031 mmol) in MeOH (1 ml), 1N NaOH aq. (0.2 ml, 0.2 mmol) was added at room temperature and stirred at room temperature for 4 h. To the mixture, ethyl acetate was added and the mixture was washed with 30% NaH₂PO₄ aq., dried over MgSO₄, concentrated in vacuo, and the obtained residue was chromatographed on silica gel (ethyl acetate/hexane=3/1) to give the title compound (13 mg, 74%) as colorless oil.

¹H-NMR (CDCl₃): 0.60 (t, 6H, J=7.2 Hz), 1.60-1.75 (m, 2H), 1.76-1.90 (m, 2H), 2.06 (q, 4H, J=7.2 Hz), 2.32 (s, 3H), 2.35 (s, 3H), 2.39 (t, 2H, J=7.4 Hz), 2.47 (t, 2H, J=6.9 Hz), 6.08 (d, 1H, J=16.0 Hz), 6.85-7.02 (m, 4H), 7.20-7.27 (m, 1H), 7.30-7.40 (m, 2H);MS(ESI+): 569([M+H]⁺)

### Example 276

### Preparation of (R)-2-[2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-acetylamino]-propionic acid

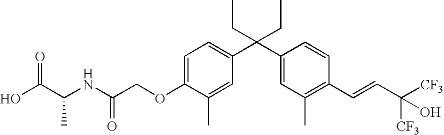

### (1) Preparation of (4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-acetic acid methyl ester

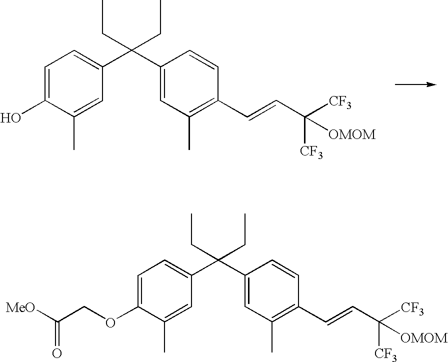

Using the same procedure as described for the preparation of Example 203-(1), the title compound was prepared from 4-{1-Ethyl-1-[3-methyl-4-((E)-4,4,4-trifluoro-3-methoxymethoxy-3-trifluoromethyl-but-1-enyl)-phenyl]-propyl}-2-methyl-phenol (compound prepared in 30-(1)).

¹H-NMR(CDCl₃): 0.60 (t, 6H), 2.04 (q, 4H), 2.23 (s, 3H), 2.32 (s, 3H), 3.50 (s, 3H), 3.80 (s, 3H), 4.62 (s, 2H), 4.96 (s, 2H), 6.06 (d; 1H), 6.57 (d, 1H), 6.88-7.05 (m, 4H), 7.30-7.38 (m,2H)

### (2) Preparation of (4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-acetic acid

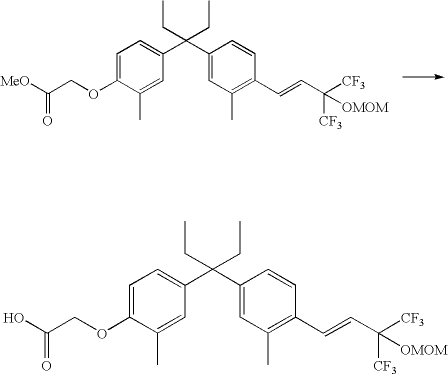

Using the same procedure as described for the preparation of Example 203-(2), the title compound was prepared from (4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-acetic acid methyl ester (compound prepared in 276-(1)).

¹H-NMR(CDCl₃): 0.56 (t, 6H), 2.01 (q, 4H), 2.15 (s, 3H), 2.28 (s, 3H), 3.48 (s, 3H), 4.46 (s, 2H), 4.94 (s, 2H), 6.04 (d, 1H), 6.57 (d, 1H), 6.85-7.01 (m, 4H), 7.28-7.37 (m, 2H)

### (3) Preparaton of (R)-2-[2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-acetylamino]-propionic acid

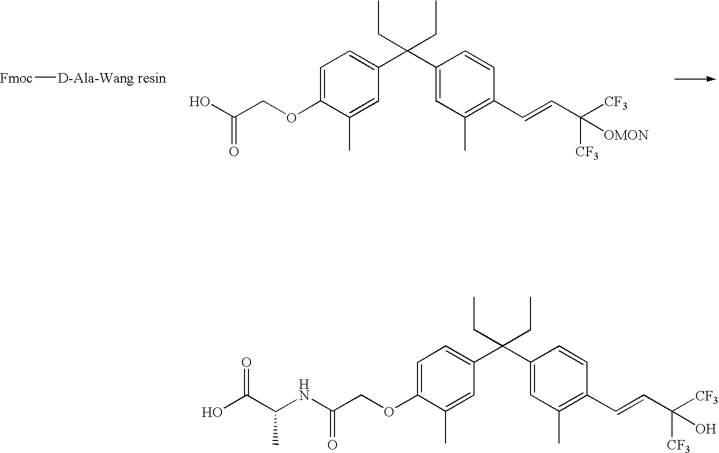

Fmoc-D-Ala-Wang resin (0.54 mmol/g as amino acid loading, 37.0 mg, 0.020 mmol amino acid loading) was treated with 20% (v/v) piperidine in DMF (0.5 ml) two times to deprotect Fmoc group and washed with DMF (1 ml, 5 times). To the resin, solutions of (4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-acetic acid (prepared in Example 276-(2)) (16.3 mg, 0.029 mmol) in DMF (0.2 ml), benzotriazole-1-yl-oxy-tris(dimethylamino)phosphonium hexafluorophosphate (17.7 mg, 0.040 mmol) in DMF (0.2 ml) and N,N-diisopropyl ethyl amine (0.0105 ml, 0.060 mmol) in DMF (0.1 ml) were added at room temperature, agitated at room temperature for 16 h, filtrated, washed with DMF (2 ml, 3 times), iPrOH (2 ml, 3 times), THF (2 ml, 3 times), MeOH (2 ml, 3 times) and CH2Cl2 (2 ml, 3 times) and dried in vacuo. To the resin, 20% TFA in CH2Cl2 (v/v) (1 ml) was added at room temperature, agitated at room temperature for 16 h, filtrated, washed with CH2Cl2 (1 ml, 2 times) and concentrated under vacuum to give the crude titled compound.

MS (positive mode): 590 ([M+H]⁺)

### Example 277

### Preparation of (R)-2-[2-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-acetylamino]-propionic acid

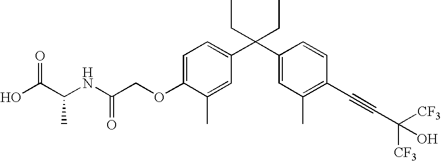

### (1) Preparation of (4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-acetic acid methyl ester

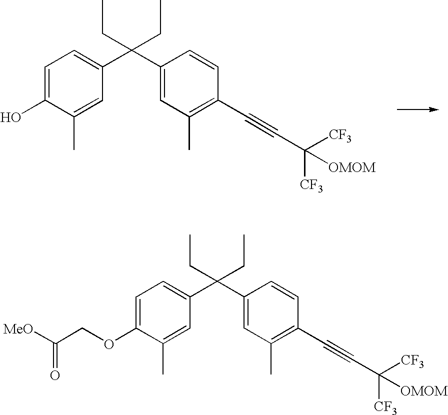

Using the same procedure as described for the preparation of Example 203-(1), the title compound was prepared from 4-{1-Ethyl-1-[3-methyl-4-(4,4,4-trifluoro-3-methoxymethoxy-3-trifluoromethyl-but-1-ynyl)-phenyl]-propyl}-2-methyl-phenol (compound prepared in 2-(2)).

¹H-NMR(CDCl₃): 0.59 (t, 6H), 2.04 (q, 4H), 2.21 (s, 3H), 2.39 (s, 3H), 3.47 (s, 3H), 3.80 (s, 3H), 4.62 (s, 2H), 5.15 (s, 2H), 6.57 (s, 1H), 6.83-7.05 (m, 4H), 7.36 (d, 1H)

### (2) Preparation of (4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-acetic acid

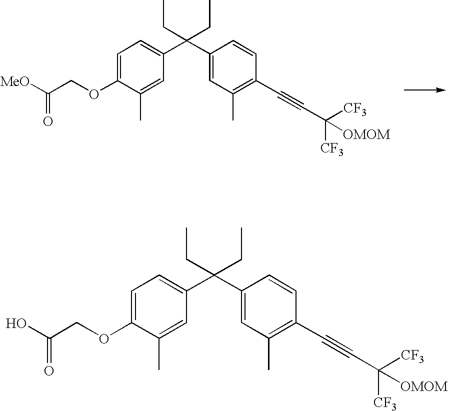

Using the same procedure as described for the preparation of Example 200-(2), the title compound was prepared from (4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-acetic acid methyl ester (compound prepared in 277-(1)).

¹H-NMR(CDCl₃): 0.59 (t, 6H), 2.04 (q, 4H), 2.16 (s, 3H), 2.39 (s, 3H), 3.48 )s, 3H), 4.65 (s, 2H), 5.15 (s, 2H), 6.61 (d, 1H), 6.85-7.05 (m, 4H), 7.37 (d, 1H)

### (3) Preparaton of (R)-2-[2-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-acetylamino]-propionic acid

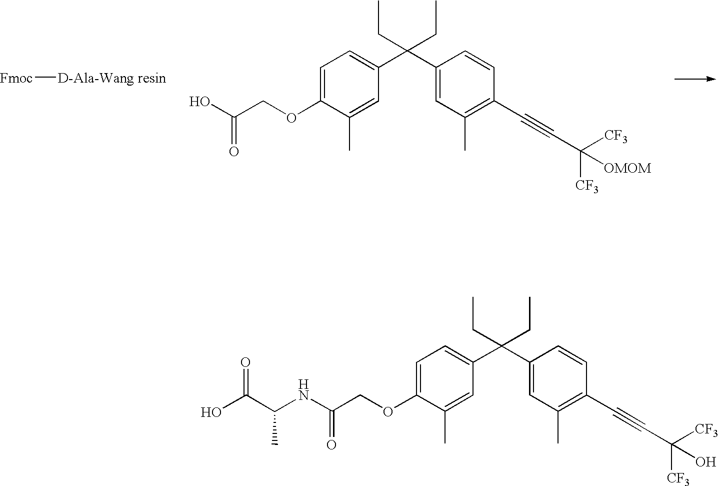

Using the same procedure as described for the preparation of Example 276-(3), the crude title compound was prepared from Fmoc-D-Ala-Wang resin and (4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-acetic acid (compound prepared in 277-(2))

MS (positive mode): 588 ([M+H]⁺)

### Example 278

### Preparation of (S)-2-[2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-acetylamino]-3-phenyl-propionic acid

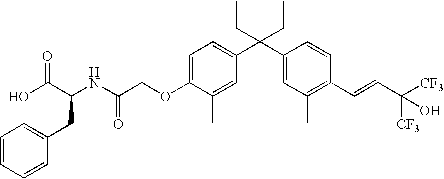

Using the same procedure as described for the preparation of Example 276-(3), the crude title compound was prepared from Fmoc-L-Phe-Wang resin and (4-{1-Ethyl-1-[3-methyl-4-((E)-4,4,4-trifluoro-3-methoxymethoxy-3-trifluoromethyl-but-1-enyl)-phenyl]-propyl}-2-methyl-phenoxy)-acetic acid (compound prepared in 276-(2))

MS (positive mode): 666 ([M+H]⁺)

### Example 279

### Preparation of (S)-2-[2-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-acetylamino]-3-phenyl-propionic acid

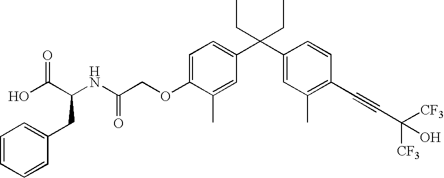

Using the same procedure as described for the preparation of Example 276-(3), the crude title compound was prepared from Fmoc-L-Phe-Wang resin and (4-{1-Ethyl-1-[3-methyl-4-(4,4,4-trifluoro-3-methoxymethoxy-3-trifluoromethyl-but-1-ynyl)-phenyl]-propyl}-2-methyl-phenoxy)-acetic acid (compound prepared in 277-(2))

MS (positive mode): 664 ([M+H]⁺)

### Example 280

### 3-(4-{1-Ethyl-1-[4-(3-hydroxy-4,4-dimethyl-pentyl)-3-methyl-phenyl]-propyl}-2-methyl-phenoxy)-propane-1,2-diol

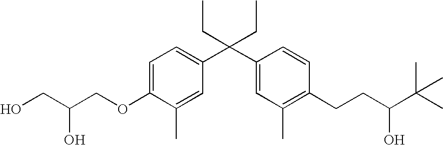

### Example 281

### 3-(4-{1-Ethyl-1-[4-(2-hydroxy-3,3-dimethyl-butyl)-3-methyl-phenyl]-propyl}-2-methyl-phenoxy)-propane-1,2-diol

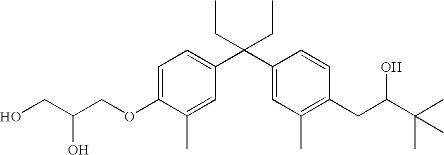

### Example 282

### (R)-3-(4-{1-Ethyl-1-[4-((R)-3-hydroxy-4,4-dimethyl-pentyl)-3-methyl-phenyl]-propyl}-2-methyl-phenoxy)-propane-1,2-diol

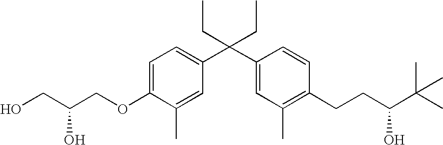

### Example 283

### (S)-3-(4-{1-Ethyl-1-[4-((S)-3-hydroxy-4,4-dimethyl-pentyl)-3-methyl-phenyl]-propyl}-2-methyl-phenoxy)-propane-1,2-diol

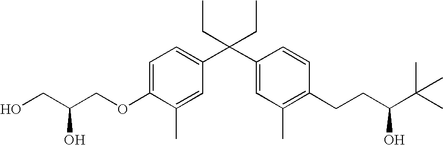

### Example 284

### (R)-3-(4-{1-Ethyl-1-[4-((S)-3-hydroxy-4,4-dimethyl-pentyl)-3-methyl-phenyl]-propyl}-2-methyl-phenoxy)-propane-1,2-diol

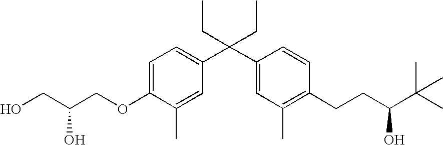

### Example 285

### (S)-3-(4-{1-Ethyl-1-[4-((R)-3-hydroxy-4,4-dimethyl-pentyl)-3-methyl-phenyl]-propyl}-2-methyl-phenoxy)-propane-1,2-diol

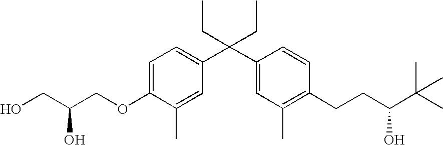

### Example 286

### (R)-3-(4-(1-Ethyl-1-[4-((E)-3-hydroxy-4,4-dimethyl-pent-1-enyl)-phenyl]-propyl}-2-methyl-phenoxy)-propane-1,2-diol

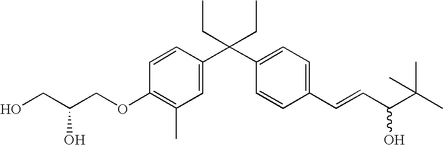

### Example 287

### (S)-3-(4-{1-Ethyl-1-[4-((E)-3-hydroxy-4,4-dimethyl-pent-1-enyl)-phenyl]-propyl}-2-methyl-phenoxy)-propane-1,2-diol

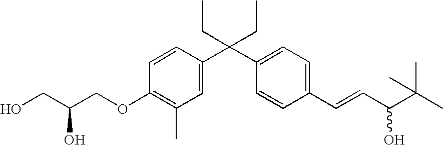

### Example 288

### (R)-3-(4-{1-Ethyl-1-[4-(3-hydroxy-4,4-dimethyl-pentyl)-phenyl]-propyl}-2-methyl-phenoxy)-propane-1,2-diol

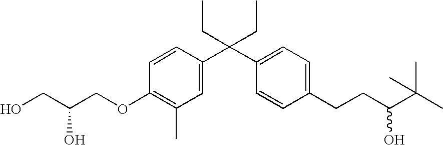

### Example 289

### (S)-3-(4-{1-Ethyl-1-[4-(3-hydroxy-4,4-dimethyl-pentyl)-phenyl]-propyl}-2-methyl-phenoxy)-propane-1,2-diol

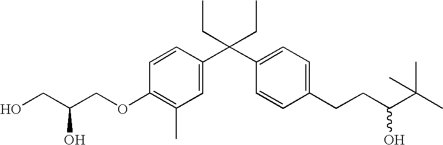

### Example 290

### (S)-5-(4-{1-Ethyl-1-[4-(3-hydroxy-4,4-dimethyl-pentyl)-3-methyl-phenyl]-propyl)-2-methyl-phenoxy)-pentane-1,4-diol

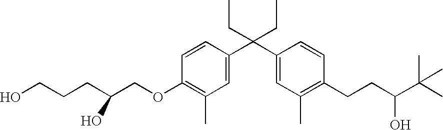

### Example 291

### 4-(4-{1-Ethyl-1-[4-(3-hydroxy-4,4-dimethyl-pentyl)-3-methyl-phenyl]-propyl}-2-methyl-phenyl)-butane-1,2-diol

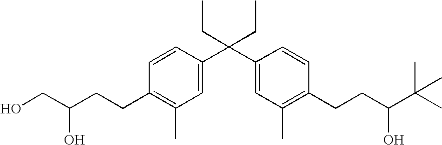

### Example 292

### 4-(4-{1-Ethyl-1-[4-(2-hydroxy-3,3-dimethyl-butoxy)-3-methyl-phenyl]-propyl}-2-methyl-phenyl)-butane-1,2-diol

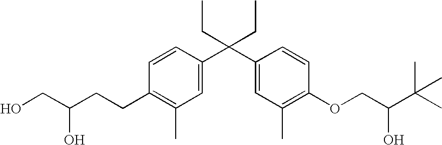

### Example 293

### 1-(4-{1-Ethyl-1-[4-(3-hydroxy-propyl)-3-methyl-phenyl]-propyl}-2-methyl-phenoxy)-3,3-dimethyl-butan-2-ol

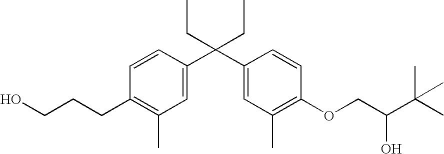

### Example 294

### 1-(4-{1-Ethyl-1-[4-(3-hydroxy-propyl)-3-methyl-phenyl]-propyl}-2-methyl-phenyl)-4,4-dimethyl-pentan-3-ol

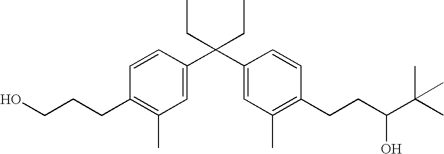

### Example 295

### 3-(4-{1-Ethyl-1-[4-(2-hydroxy-3,3-dimethyl-butoxy)-3-methyl-phenyl]-propyl}-2-methyl-phenyl)-butan-1-ol

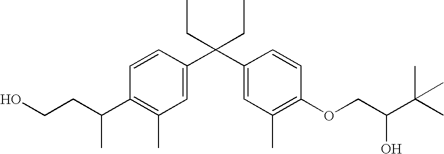

### Example 296

### (E)-4-(4-{1-Ethyl-1-[4-(2-hydroxy-3,3-dimethyl-butoxy)-3-methyl-phenyl]-propyl}-2-methyl-phenyl)-but-3-en-2-ol

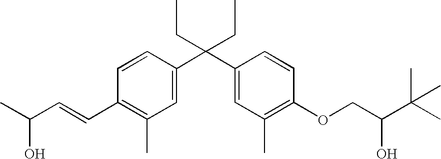

### Example 297

### 1-(4-{1-Ethyl-1-[4-(3-hydroxy-butyl)-3-methyl-phenyl]-propyl}-2-methyl-phenoxy)-3,3-dimethyl-butan-2-ol

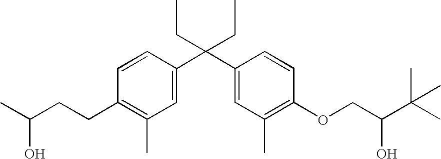

### Example 298

### 3-(4-{1-Ethyl-1-[4-(2-hydroxy-3,3-dimethyl-butoxy)-3-methyl-phenyl]-propyl}-2-methyl-phenyl)-propane-1,2-diol

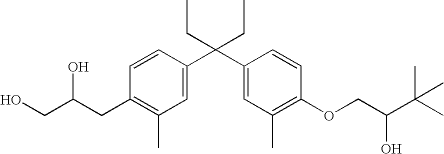

### Example 299

### 3-(4-{1-Ethyl-1-[4-(2-hydroxy-3,3-dimethyl-butoxy)-3-methyl-phenyl]-propyl}-2-methyl-phenyl)-butane-1,2-diol

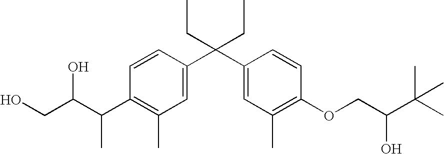

### Example 300

### 1-(4-{1-[4-(2,3-Dihydroxy-propyl)-3-methyl-phenyl]-1-ethyl-propyl}-2-methyl-phenoxy)-3,3-dimethyl-butan-2-one

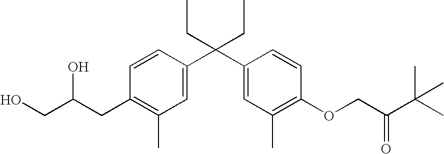

### Example 301

### 1-(4-{1-[4-(2,3-Dihydroxy-1-methyl-propyl)-3-methyl-phenyl]-1-ethyl-propyl}-2-methyl-phenoxy)-3,3-dimethyl-butan-2-one

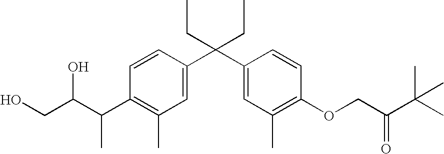

### Example 302

### 1-(4-{1-[4-(1,2-Dihydroxy-propyl)-3-methyl-phenyl]-1-ethyl-propyl}-2-methyl-phenoxy)-3,3-dimethyl-butan-2-one

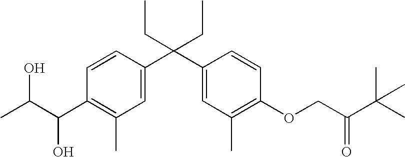

### Example 303

### 1-(4-{1-Ethyl-1-[4-(3-hydroxy-4,4-dimethyl-pentyl)-3-methyl-phenyl]-propyl-2-methyl-phenyl)-4,4-dimethyl-pentan-3-ol

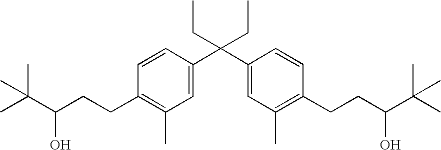

The following compounds are prepared in accordance with the procedure described as in the above Examples.

### Example 304

### Preparation of (R)-5-[4-(1-Ethyl-1-{4-[3-hydroxy-3-(1-methyl-cyclopentyl)-prop-1-ynyl]-3-methyl-phenyl)-propyl)-2-methyl-phenoxymethyl]-dihydro-furan-2-one

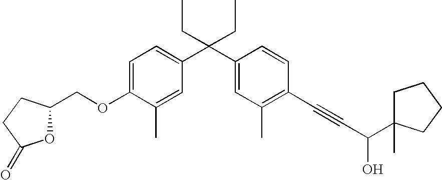

### Example 305

### Preparation of 5-[4-(1-Ethyl-1-{4-[3-hydroxy-3-(1-methyl-cyclopentyl)-prop-1-ynyl]-3-methyl-phenyl}-propyl)-2-methyl-phenoxy]-4(R)-hydroxy-pentanoic acid

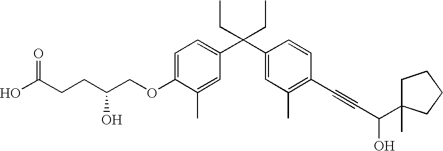

### Example 306

### Preparation of (R)-5-[4-(1-ethyl-1-{4-[3-hydroxy-3-(1-methyl-cyclopentyl)-propyl]-3-methyl-phenyl}-propyl)-2-methyl-phenoxymethyl]-dihydro-furan-2-one

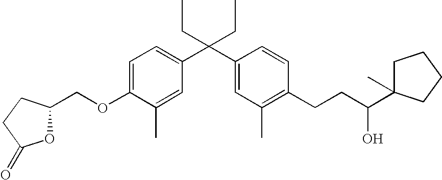

### (1) Preparation of 1-methyl-cyclopentanecarboxylic acid N-methoxy-N-methyl-amide

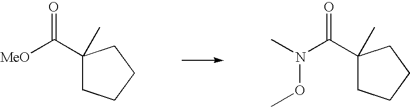

To a solution of N,O-dimethylhydroxylamine hydrochloride (594.5 mg, 6.09 mmol) in THF (4 ml) was added 2.67M n-butyl lithium in hexane (4.10 ml, 10.95 mmol) dropwise at −78 degrees C., and the mixture was stirred at room temperature for 10 min. The mixture was cooled to −78 degrees C., and 1-methyl-cyclopentanecarboxylic acid methyl ester (430.5 mg, 3.03 mmol) in THF (2 ml) was added dropwise to the mixture. After stirring at −78 degrees C. for 1 h 45 min, diethyl ether was added, and the solution was washed with sat.NH₄Cl aq. and brine, dried over MgSO₄, filtered, concentrated in vacuo, and chromatographed (ethyl acetate/hexane=0/100 to 15/85) to give the title compound (154.2 mg, 30%).

1H-NMR (CDCl₃): 1.23 (s, 3H), 1.55-1.66 (m, 6H), 2.03-2.08 (m, 2H), 3.18 (s, 3H), 3.66 (s, 3H); MS(ESI+): 172([M+H]⁺).

### (2) Preparation of 3-(4-1-[4-(tert-butyl-dimethyl-silanyloxy)-3-methyl-phenyl]-1-ethyl-propyl}-2-methyl-phenyl)-1-(1-methyl-cyclopentyl)-prop-2-yn-1-one

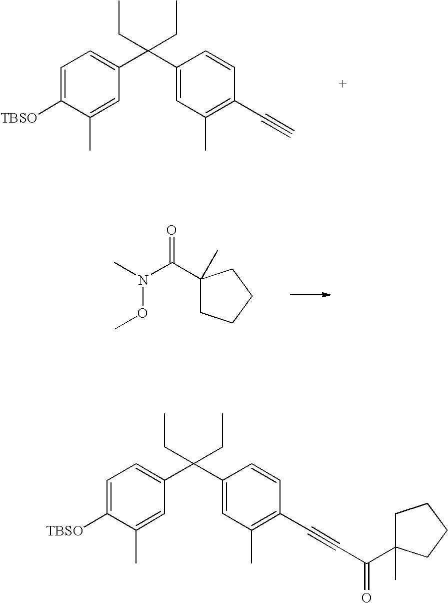

To a solution of tert-butyl-{4-[1-ethyl-1-(4-ethynyl-3-methyl-phenyl)-propyl]-2-methyl-phenoxy}-dimethyl-silane (compound prepared in Example 260-(2)) (434.1 mg, 1.07 mmol) in THF (3 ml), 2.67 M n-butyl lithium in hexane (0.420 ml, 1.12 mmol) was added at −78 degrees C. To the mixture, 1-methyl-cyclopentanecarboxylic acid N-methoxy-N-methyl-amide (compound prepared in Example 306-(1)) (151.9 mg, 0.887 mmol) in THF (2 ml) was added at −78 degrees C., stirred at −78 degrees C. to room temperature for 2.5 h. The mixture was poured into brine and extracted with ethyl acetate. Organic layer was washed with brine, dried over MgSO₄, filtered, concentrated in vacuo, and chromatographed (ethyl acetate/hexane=0/100 to 2/98) to give the title compound (434.9 mg, 95 %).

1H-NMR (CDCl₃): 0.19 (s, 6H), 0.58 (t, 6H, J=7.3 Hz), 0.99 (s, 9H), 1.32 (s, 3H), 1.42-1.51 (m, 2H), 1.69-1.74 (m, 4H), 2.03 (q, 4H, J=7.2 Hz), 2.14 (s, 3H), 2.24-2.32 (m, 2H), 2.44 (s, 3H), 6.63 (d, 1H, J=8.4 Hz), 6.79 (dd, 1H, J=8.4, 2.0 Hz), 6.84 (s, 1H), 7.00 (d, 1H, J=8.9 Hz), 7.05 (s, 1H), 7.41 (d, 1H, J=8.1 Hz);

MS(ESI+): 517([M+H]⁺).

### (3) Preparation of 4-(1-ethyl-1-{4-[3-hydroxy-3-(1-methyl-cyclopentyl)-prop-1-ynyl]-3-methyl-phenyl}-propyl)-2-methyl-phenol

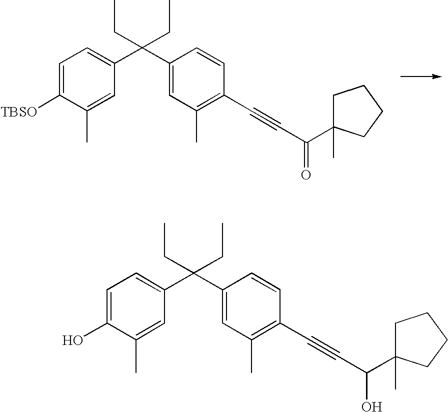

To a solution of 3-(4-{1-[4-(tert-butyl-dimethyl-silanyloxy)-3-methyl-phenyl]-1-ethyl-propyl}-2-methyl-phenyl)-1-(1-methyl-cyclopentyl)-prop-2-yn-1-one (compound prepared in Example 306-(2)) (432 mg, 0.835 mmol) in MeOH (3 ml) and THF (8 ml), NaBH₄ (155.5 mg, 4.11 mmol) was added at room temperature, stirred at room temperature for 30 min. Ethyl acetate was added. The solution was washed with brine, dried over MgSO₄, concentrated in vacuo. The residue was dissolved to THF (16 ml), and 1M terabutylammonium fluoride in THF (0.880 ml, 0.880 mmol) was added at −10 degrees C. The mixture was stirred for 5 min. Ethyl acetate was added. The solution was washed with brine, dried over MgSO₄, concentrated in vacuo, and purified by preparative TLC (ethyl acetate/hexane=0/100 to 25/75) to give the title compound (352 mg, quant.).

1H-NMR (CDCl₃): 0.58 (t, 6H, J=7.3 Hz), 1.14 (s, 3H), 1.37-1.44 (m, 2H), 1.57-1.80 (m, 6H), 1.82 (d, 1H, J=5.9 Hz), 2.02 (q, 4H, J=7.3 Hz), 2.18 (s, 3H), 2.36 (s, 3H), 4.38 (d, 1H, J=5.9 Hz), 4.51 (s, 1H), 6.63 (dd, 1H, J=8.8, 1.5 Hz), 6.81-6.85 (m, 2H), 6.92 (dd, 1H, J=8.1, 1.6 Hz), 6.98 (s, 1H), 7.27 (d, 1H, J=8.3 Hz);

MS(ESI+): 405([M+H]⁺).

### (4) Preparation of (R)-5-[4-(1-ethyl-1-{4-[3-hydroxy-3-(1-methyl-cyclopentyl)-propyl]-3-methyl-phenyl}-propyl)-2-methyl-phenoxymethyl]-dihydro-furan-2-one

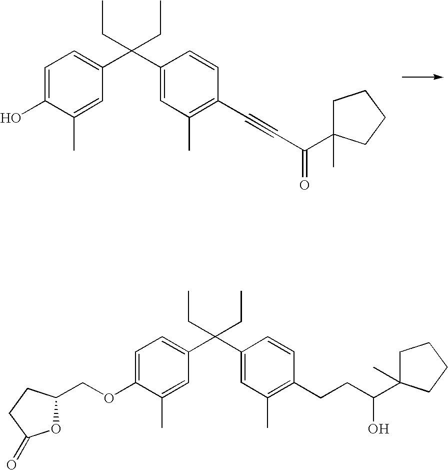

To a solution of 4-(1-ethyl-1-{4-[3-hydroxy-3-(1-methyl-cyclopentyl)-prop-1-ynyl]-3-methyl-phenyl}-propyl)-2-methyl-phenol (compound prepared in Example 306-(3)) (140 mg, 0.346 mmol) in MeOH (15 ml), 10% Pd/C (31.1 mg) was added. The mixture was stirred at room temperature for 16 h under hydrogen. The mixture was filtered, concentrated in vacuo. To the residue in N,N-dimethylacetamide (5 ml), K₂CO₃ (194 mg, 1.40 mmol) and (R)-(−)-dihydro-5-(p-tolylsulfonyloxymethyl)-2(3H)-furanone (152 mg, 0.562 mmol) were added. The mixture was stirred at 90 degrees C. for 27 h. After cooled to room temperature, (R)-(−)-dihydro-5-(p-tolylsulfonyloxymethyl)-2(3H)-furanone (10.2 mg, 0.0377 mmol) was added. The mixture was stirred at 90 degrees C. for 6.5 h. After cooled to room temperature, brine was added, extracted with ethyl acetate. The organic layer was washed with brine, dried over MgSO₄, concentrated in vacuo, and chromatographed (ethyl acetate/hexane=0/100 to 40/60) to give the title compound (109.1 mg, 62%).

1H-NMR (CDCl₃): 0.58 (t, 6H, J=7.3 Hz), 0.90 (s, 3H), 1.23-1.40 (m, 4H), 1.49-1.73 (m, 6H), 2.03 (q, 4H, J=7.2 Hz), 2.14 (s, 3H), 2.25 (s, 3H), 2.28-2.62 (m, 4H), 2.71-2.87 (m, 2H), 3.30-3.34 (m,1 H), 4.11 (ddd, 2H, J=28.2, 8.4, 3.29 Hz), 4.854.90 (m, 1H), 6.65 (d, 1H, J=8.4 Hz), 6.88-7.03 (m, 5H); MS(ESI−): 505([M−H]⁻).

### Example 307

### Preparation of 5-[4-(1-Ethyl-1-{4-[3-hydroxy-3-(1-methyl-cyclopentyl)-propyl]-3-methyl-phenyl}-propyl)-2-methyl-phenoxy]-4(R)-hydroxy-pentanoic acid

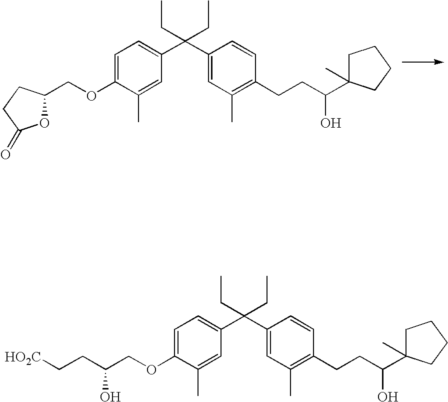

To a solution of (R)-5-[4-(1-ethyl-1-{4-[3-hydroxy-3-(1-methyl-cyclopentyl)-propyl]-3-methyl-phenyl}-propyl)-2-methyl-phenoxymethyl]-dihydro-furan-2-one (compound prepared in Example 306-(4)) (105.7 mg, 0.209 mmol) in MeOH (10 ml) and THF (10 ml), 1N NaOH solution (2 ml) was added. The mixture was stirred at room temperature for 2 h. The mixture was concentrated in vacuo and purified by preparative TLC (CHCl₂/MeOH=8/3 saturated with H₂O) to give the title compound (86.1 mg, 79%).

1H-NMR (CDCl₃): 0.58 (t, 6H, J=7.2 Hz), 0.89 (s, 3H), 1.20-1.80 (m,10H), 1.87-1.93 (m, 2H), 2.03 (q, 4H, J=7.2 Hz), 2.16 (s, 3H), 2.25 (s, 3H), 2.50-2.63 (m, 3H), 2.79-2.90 (m, 1H), 3.33 (dd, 1H, J=10.0, 1.6 Hz), 3.83 (dd, 1H, J=9.6, 6.8 Hz), 3.95 (dd, 1H, J=9.6, 3.5 Hz), 4.00-4.10 (m, 1H), 6.66 (d, 1H, J=8.3 Hz), 6.88-6.95 (m, 4H), 7.01 (d, 1H, J=8.4 Hz); MS(ESI−): 523([M−H]³¹).

### Example 308

### Preparation of (S)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one

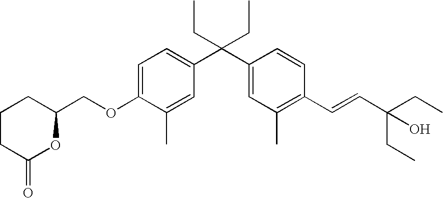

### Preparation of (S)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one

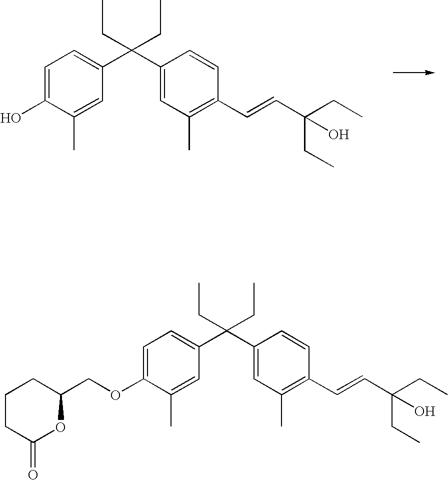

To a solution of 4-{1-ethyl-1-[4-((E)-3-ethyl-3-hydroxy-pent-1-enyl)-3-methyl-phenyl]-propyl)-2-methyl-phenol (compound prepared in Example 1-(5)) (50 mg, 0.131 mmol), PPh₃ (103 mg, 0.394 mmol), (S)-6-hydroxymethyl-tetrahydro-pyran-2-one (J. Chem. Soc., Perkin Trans. 1, 2000, 20, 3519.) (51 mg, 0.394 mmol) in THF (0.2 ml), DEAD (69 mg, 0.394 mmol) was added slowly at room temperature and stirred at room temperature for 37 h. The mixture was concentrated in vacuo and chromatographed (n-hexane/ethyl acetate=1/3 developed once, n-hexane/ethyl acetate=3/1 developed once, n-hexane/ethyl acetate =1/1 developed once) to give the title compound (5.3 mg, 8.2%).

MS(positive): 510([M+NH₄]⁺).

### Example 309

### Preparation of (S)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid

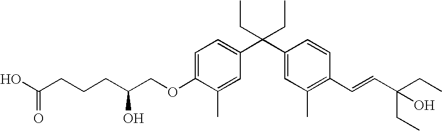

### Preparation of (S)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid

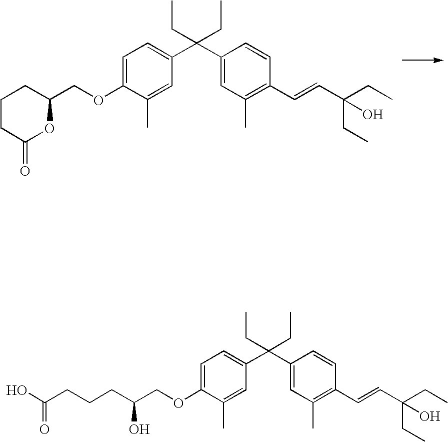

To a solution of (S)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one (compound prepared in Example 308) (0.53 mg, 0.001 mmol) in THF (0.05 ml) and, MeOH (0.05 ml), 1N KOH aq (0.05 ml, 0.05 mmol) was added at rt and stirred at 60 degrees C. for 12 h. The mixture was extracted with ethyl acetate, dried over MgSO₄, filtered, concentrated in vacuo to give the title compound.

MS(positive): 493([M−OH]⁺), MS(negative): 509([M−H]⁻).

### TEST EXAMPLE

Biological Activities

Preparation of Tested Compounds

1,25(OH)₂D₃ was purchased from Solvay Pharmaceuticals (Weesp, The Netherlands). LG190178 (the compound disclosed in WO00/10958 and the corresponding U.S. Pat. No. 6,218,430 B1) was prepared in accordance with the procedure described as the said patent specification (Example 10). Compound A, B, C, and D (the compounds disclosed in WO03/101978A1) were prepared in accordance with the procedure described as the said patent specification (Example 110 and its diastereomers). The compounds of the present invention were prepared as exemplified above.

The chemical structure of LG190178 is as follows:

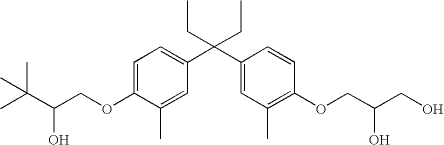

The chemical structure of compound A is as follows:

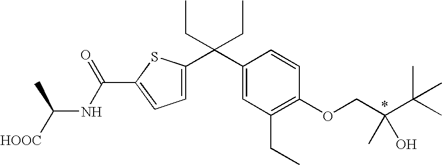

The chemical structure of compound B (epimer of compound A) is as follows:

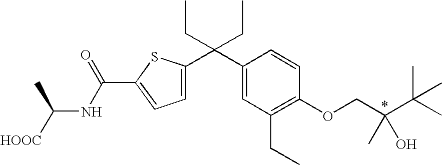

The chemical structure of compound C is as follows:

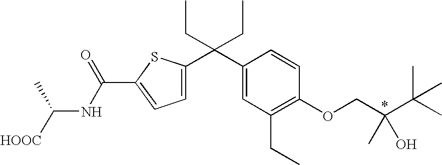

The chemical structure of compound D (epimer of compound C) is as follows:

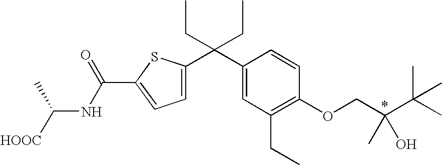

### Test Example 1

### PTH Production From Bovine Parathyroid Cells

Preparation of Parathyroid Cells

Fresh bovine parathyroid glands obtained from a slaughterhouse were transported to the laboratory in ice-cold DMEM/F-12 (GIBCO BAL, NY, USA) containing antibiotic-antimycotic (GIBCO BAL). The parathyroid glands were trimmed of excess fat and connective tissue with scissors and sliced with the slicer. They were minced finely (not more than 1 mm fragments) and suspended in digestion medium consisting of DMEM/F-12 (10 ml digestion medium/1 g parathyroid tissue) containing 2 mg/ml collagenase (Wako Pure Chemical Industries, Ltd., Osaka, Japan), 40 mg/ml deoxyribonuclease 11 (SIGMA-ALDRICH Co., St. Louis Mo., USA), and antibiotics (penicillin, streptomycin). The mixture was incubated at 37° C. with shaking (120 strokes/min.) for 120-180 min. to disperse the cells. The turbid solution was then filtered through nylon mesh (70 μm cell strainer) and centrifuged at 1100 rpm. (240×g) for 10 min. at 4° C. The pellet was resuspended and washed twice in DMEM/F-12. The pellet was finally resuspended in DMEM/F-12 containing 10% fetal bovine serum (FBS) and aliquots were taken for cell counting.

Parathyroid Cell Cultures

The dispersed cells were plated in 96-well plate at a concentration of 2×10⁴ cells/well and incubated for 2 days at 37° C. The culture medium was then replaced with the same medium containing various concentrations of 1,25(OH)2D₃ (as a positive control), LG190178 (the compound disclosed in WO00/10958 and the corresponding U.S. Pat. No. 6,218,430 B1) or the compounds of the present invention, and cells were cultured for 3 days at 37° C. To determine the rate of PTH secretion, the cells were washed once with DMEM/F-12 (FBS free), and then incubate for 4 hr. in fresh FBS free medium at 37° C. The medium were collected and frozen at −30° C. until assay for the concentration of PTH.

Radio Immuno Assay of PTH in the Medium

PTH concentration in the culture media was determined using the PTH assay kit YAMASA RIA kit available from YAMASA Co., (Chiba, Japan).

Comparison of Inhibitory Activity on PTH Release in Parathyroid Cells

The 50% inhibitory concentration (IC₅₀) values were determined from the PTH concentrations. The inhibitory activity was calculated as the ratio of the IC₅₀ value of the compounds to that of 1,25(OH)₂D₃. The results are shown in the following Table 2. The relationship between the diseases and the inhibitory activity on PTH release in parathyroid cells is described in Am J Kidney Dis. November 1995; 26(5):852-60 for example.

### Test Example 2

### The mRNA Expression Assay of ECAC2 in Caco-2 Cells

Caco-2 Cell Cultures

The dispersed cells were plated in 48-well plate at a concentration of 6.3×10⁴ cells/well and incubated for 4 days at 37° C. The culture medium (Dulbecco's Modified Eagle Medium (D-MEM) without NaHCO₃ with 44 mM NaHCO₃, 1 mM non-essential amino acids, and 2 mM L-glutamine, 90%; fetal bovine serum, 10%) was then replaced with the same medium containing various concentrations of LG190178 (the compound disclosed in WO00/10958 and the corresponding U.S. Pat. No. 6,218,430 B1) or the compounds of the present invention, and cells were cultured for 1 day at 37° C. The culture medium containing the compounds was changed twice per day.

Total RNA Isolation from Caco-2 Cell

Total RNA was isolated from Caco-2 cell following ABI PRISM™ 6100 Nucleic Acid PrepStation protocol from Applied Biosystems.

Real Time RT-PCR Assay of ECAC2

ECAC2 mRNA expression was determined using TaqMan One-step RT-PCR Master Mix Reagents Kit and PRISM7000 systems from Applied Biosystems.

Primer information and cycle conditions are 1)ECAC2, forward 5′-CCTTCACCATCATGATTCAGMG-3′, reverse 5′-GTCCTCTGTCTGGMGATGA-3′, Taqman probe 5′-TTCTGCTGGCTGATGGCTGTGGTC-3′, 55 cycles, annealing temperature (Ta)=60° C.; 2)GAPDH, forward 5′-GMGGTGMGGTCGGAGTC-3′, reverse 5′-GMGATGGTGATGGGATTTC-3′, Taqman probe 5′-CAAGCTTCCCGTTCTCAGCC-3′, 55 cycles, Ta=60° C. ECAC2 mRNA values were normalized for the expression of GAPDH mRNA within each sample.

Comparison of ECAC2 mRNA Value

The EC₅₀ values of ECAC2 mRNA values were analyzed by Emax models. The ECAC2 mRNA ratio was calculated as the ratio of the EC₅₀ value of the compounds to that of LG190178. The results are shown in the following Table 3. ECAC2 is a channel-like transporter mediating intestinal calcium absorption. It is described in J Biol Chem. Aug. 6, 1999; 274(32):22739-46 for example.

### Test Example 3

### MG-63 Osteocalcin Induction Assay

Preparation of MG-63 Cells

MG-63 cells were cultured in MEM (minimum essential medium) medium (pH 7.0) (Invitrogen Corp., Carlsbad, Calif., USA) containing 5% fetal bovine serum (FBS) (Hyclone, Logan, Utah, USA) before assay. MG-63 cells were detached by Trypsin-EDTA solution and resuspended in MEM medium. Cell density was adjusted to 4.0×10⁴ cells/mL with the MEM medium and 0.2 mL of the cells was seeded onto each well of 96-well plate. The cells were incubated at 37° C. in 5% CO₂ for 24 hours.

MG-63 Cells Culture

The cells were washed once with 200 micro L of MEM medium containing 5% of DCC-FBS. Then, the 180 micro L of MEM medium containing 5% dextran/charcoal-treated FBS (DCC-FBS) were added onto each well of 96-well plate.

1,25(OH)₂D₃ was purchased from Solvay Pharmaceuticals (Weesp, The Netherlands) and was used for standard. Test compounds were diluted to 1.0×10⁻³ mol/L, 1.0×10⁻⁴ mol/L, 1.0×10⁻⁵ mol/L, 1.0×10⁻⁶ mol/L, 1.0×10⁻⁷ mol/L and 1.0×10⁻⁸ mol/L with DMSO. These compounds solutions were stored at −20° C. until use. MG-63 cells were treated with these compounds (20 micro L) at final concentration of 1.0×10⁻⁶ mol/L, 1.0×10⁻⁷ mol/L, 1.0×10⁻⁸ mol/L, 1.0×10⁻⁹ mol/L, 1.0×10⁻¹⁰ mol/L and 1.0×10⁻¹¹ mol/L in MEM medium containing 5% DCC-FBS. The plate was mixed gently and incubated at 37° C. and 5% CO₂ for 8 hours. The final concentration of DMSO was less than 0.1%. After the incubation, cultured medium were removed and 200 micro L of MEM containing 5% DCC-FBS were added onto each well of 96-well plate. The cells were incubated at 37° C. and 5% CO₂ for 4 days.

After 4 days culture, 150 micro L of supernatant was collected into new 96-well plate and stored at −80° C.

Enzyme Immunoassay of Osteocalcin

Osteocalcin concentration of supernatant was determined by Gla-type osteocalcin EIA kit (Takara Bio. Inc., Tokyo, Japan). Frozen supernatant was thawed slowly with shaking gently at room temperature prior to use. The absorbance was measured at 450 nm with a microplate reader (Model 3550, Bio-Rad Laboratories, Hercules, Calif., USA). Each concentration was calculated by comparison with standards using Microplate Manager III software (Bio-Rad Laboratories).

Comparison of Induction Activity on Osteocalcin in MG-63 Cells

The 50 % effect concentration (EC₅₀) values were determined from the osteocalcin concentrations. The inductive activity was calculated as the ratio of the EC₅₀ value of the compounds to that of 1,25(OH)₂D₃. The results are shown in the following Table 4. The relationship between the diseases and the induction activity on osteocalcin is suggested in Lancet (May 19, 1984). 1(8386), 1091-3. Steroids 66, 159-170 (2001) or Journal of Steroid Biochemistry & Molecular Biology 89-90, 269-271 (2004) for example.

### Test Example 4

### Bone Mineral Density in the Ovariectomized (OVX) Rats (1)

Reagents

The compounds of the present invention were prepared as exemplified above, dissolved in a vehicle (medium chain triglyceride, MCT) and diluted to a given concentration. The stock solutions were protected from light and stored at 4° C.

Animals

Eight-month-old female Sprague-Dawley (Crl:CD(SD)) rats were purchased from Charles River Japan Inc. (Kanagawa, Japan) and acclimated under standard laboratory conditions at 24±2° C. and 50-60% humidity. The rats were allowed free access to tap water and commercial standard rodent chow CE-2 (Clea Japan Inc., Japan).

Experimental Design

Sham-operated (n=8) and the ovariectomized (OVX) rats were used. The OVX rats were divided into each group (n=8) one day after the surgery. In the sham and OVX control groups, rats received the vehicle (MCT) p.o. at a dose of 1 ml/kg body weight (BW) five times a week. Each compound was given orally five times a week for 4 weeks. These animal studies were carried out in accordance with Chugai Pharmaceutical's ethical guidelines for animal care, and the experimental protocols were approved by the animal care committee of the institution.

During the 24 hours from the last administration, urine was collected with metabolic cage, and blood was drawn from the abdominal aorta under ether anesthesia. Blood and urine samples were centrifuged to obtain the supernatant, which were stored at −20° C. until assay. The lumbar vertebrae and femur were removed. The lumbar vertebra and the right femur were stored in 70% ethanol at 4° C. for the measurement of bone mineral density (BMD).

Serum and Urine Biochemistry

Serum calcium (Ca) and inorganic phosphorus (Pi) concentrations were measured with an autoanalyzer (Hitachi 7170, Tokyo, Japan). The serum osteocalcin concentration was measured with Osteocalcin rat ELISA system (Amersham Pharmacia Biotech, Tokyo, Japan). Urinary Ca, Pi, and creatinine (Cre) were measured with an autoanalyzer. Urinary deoxypyridinoline (D-Pyr) was measured using a Metra DPD EIA kit (Quidel Co., USA), and the data were corrected for urinary Cre concentrations.

Measurement of Bone Mineral Density

The second through fourth lumbar vertebaral (L2-L4) BMD (mg/cm²) and femoral BMD were measured by dual-energy X-ray absorptiometry (DCS-600-EX, Aloka, Japan).

The results are shown in the following Table 5.

### Test Example 5

### Bone Mineral Density in the Ovariectomized (OVX) Rats (2)

Reagents

Alfacalcidol was synthesized in Chugai Pharmaceutical Co., Ltd., Japan, dissolved in a vehicle (medium chain triglyceride, MCT) and diluted to a given concentration. The stock solutions were protected from light and stored at 4° C.

Animals

Eight-month-old female Sprague-Dawley (Crl:CD(SD)) rats were purchased from Charles River Japan Inc. (Kanagawa, Japan) and acclimated under standard laboratory conditions at 23±2° C. and 40-70% humidity. The rats were allowed free access to tap water and commercial standard rodent chow CRF-1 (Oriental Yeast Co. Ltd., Japan).

Experimental Design

Sham-operated (n=8) and the ovariectomized (OVX) rats were used. The OVX rats were divided into each group (n=8) one day after the surgery. In the sham and OVX control groups, rats received the vehicle (MCT) p.o. at a dose of 1 ml/kg body weight (BW) every day. Each compound was given orally seven times a week for 4 weeks.

During the 24 hours from the last administration, urine was collected with metabolic cage, and blood was drawn from the jugular vein under ether anesthesia. Blood and urine samples were centrifuged to obtain the supernatant, which were stored at −20° C. until assay. The lumbar vertebrae and femora were removed. The lumbar vertebra and the right femur were stored i 70% ethanol at 4° C. for the measurement of bone mineral density (BMD).

Serum and Urine Biochemistry

Serum calcium (Ca) and inorganic phosphorus (Pi) concentrations were measured with an autoanalyzer (Hitachi 7170, Tokyo, Japan). The serum osteocalcin concentration was measured with Osteocalcin rat ELISA system (Smersham Pharmacia Biotech, Tokyo, Japan). Urinary Ca, Pi, and creatinine (Cre) were measured with an autoanalyzer. Urinary deoxypyridinoline (D-Pyr) was measured using a Metra DPD EIA kit (Quidel Co., USA), and the data were corrected for urinary Cre concentrations.

Measurement of Bone Mineral Density

The second through fifth lumbar vertebaral (L2-L5) BMD (mg/cm²) and femoral BMD were measured by dual-energy X-ray absorptiometry (DCS-600-EX, Aloka, Japan).

The results are shown in the following Table 6.

TABLE 2

| | Inhibitory activity |
|---|---|
| | on PTH release in |
| | parathyroid cells |
| Example No | (%) |
| 1 | 332 |
| 2 | 431 |
| 27 | 105 |
| 30 | 1688 |
| 31 | 268 |
| 49 | 387 |
| 52 | 617 |
| 54 | 170 |
| 56 | 546 |
| 59 | 485 |
| 60 | 312 |
| 62 | 1990 |
| 69 | 931 |
| 77 | 119 |
| 78 | 765 |
| 79 | 139 |
| 84 | 618 |
| 88 | 111 |
| 92 | 251 |
| 106 | 157 |
| 107 | 734 |
| 115 | 150 |
| 154 | 178 |
| 156 | 400 |
| 157 | 175 |
| 170 | 105 |
| 192 | 182 |
| 1,25(OH)₂D₃ | 100 |
| LG190178 | 2 |
| Compound A | 3 |
| Compound B | 1 |
| Compound C | 1 |
| Compound D | 1 |

TABLE 3

| | Osteocalcin |
|---|---|
| | production activity |
| Example No | in MG-63 cells (%) |
| 1 | 447 |
| 2 | 546 |
| 27 | 302 |
| 28 | 427 |
| 30 | 3746 |
| 31 | 429 |
| 35 | 1317 |
| 37 | 238 |
| 44 | 282 |
| 48 | 919 |
| 49 | 1718 |
| 52 | 5219 |
| 54 | 119 |
| 55 | 1288 |
| 56 | 10747 |
| 57 | 341 |
| 59 | 1281 |
| 60 | 1464 |
| 61 | 14230 |
| 62 | 2677 |
| 64 | 807 |
| 66 | 337 |
| 67 | 556 |
| 68 | 1078 |
| 69 | 4906 |
| 72 | 350 |
| 75 | 363 |
| 77 | 1003 |
| 78 | 4571 |
| 79 | 329 |
| 80 | 368 |
| 81 | 454 |
| 84 | 3371 |
| 86 | 271 |
| 88 | 458 |
| 92 | 402 |
| 102 | 856 |
| 106 | 159 |
| 107 | 471 |
| 115 | 173 |
| 119 | 906 |
| 121 | 248 |
| 125 | 618 |
| 140 | 277 |
| 141 | 73 |
| 147 | 241 |
| 152 | 581 |
| 153 | 796 |
| 154 | 4289 |
| 155 | 444 |
| 156 | 4747 |
| 157 | 1959 |
| 165 | 231 |
| 166 | 1344 |
| 167 | 1037 |
| 169 | 3771 |
| 170 | 2164 |
| 186 | 888 |
| 187 | 2176 |
| 190 | 616 |
| 192 | 289 |
| 195 | 530 |
| 198 | 462 |
| 1,25(OH)₂D₃ | 100 |
| LG190178 | 185 |
| Compound A | 67 |
| Compound B | 3 |
| Compound C | 43 |
| Compound D | 4 |

TABLE 4

| | ECaC-2 mRNA |
|---|---|
| | induction activity |
| | in Caco-2 cells |
| Example No | (%) |
| 1 | 36 |
| 2 | 49 |
| 27 | 114 |
| 28 | 2093 |
| 30 | 1777 |
| 31 | 161 |
| 35 | 538 |
| 37 | 639 |
| 44 | 96 |
| 48 | 519 |
| 49 | 987 |
| 52 | 1227 |
| 54 | 172 |
| 55 | 662 |
| 56 | 276 |
| 57 | 24 |
| 59 | 117 |
| 60 | 235 |
| 61 | 918 |
| 62 | 410 |
| 64 | 90 |
| 66 | 126 |
| 67 | 860 |
| 68 | 355 |
| 69 | >10000 |
| 72 | 542 |
| 75 | 309 |
| 77 | 534 |
| 78 | 6659 |
| 79 | 178 |
| 80 | 518 |
| 81 | 102 |
| 84 | 1843 |
| 86 | 289 |
| 88 | 845 |
| 92 | 226 |
| 102 | 166 |
| 106 | 399 |
| 107 | 823 |
| 115 | 91 |
| 119 | 12 |
| 121 | 75 |
| 125 | <10 |
| 140 | 53 |
| 141 | 31 |
| 147 | 75 |
| 152 | 586 |
| 153 | 1662 |
| 154 | 1784 |
| 155 | 86 |
| 156 | 4760 |
| 157 | 1647 |
| 165 | 2017 |
| 166 | 86 |
| 167 | 323 |
| 170 | 191 |
| 186 | 57 |
| 187 | 545 |
| 190 | 22 |
| 192 | 292 |
| 195 | 135 |
| 198 | 271 |
| 1,25(OH)₂D₃ | 556 |
| LG190178 | 100 |
| Compound A | 123 |
| Compound B | 15 |
| Compound C | 34 |
| Compound D | 14 |

TABLE 5

| | Spinal | | | |
|---|---|---|---|---|
| | BMD | | serum Ca | |
| | mg/cm2 | S.E. | mg/dL | S.E. |
| SHAM | 185.8 | 4.8 | 10.4 | 0.1 |
| OVX | 166.8 | 4.3 | 10.0 | 0.1 |
| Example 140 compound | 186.6 | 5.6 | 10.0 | 0.1 |
| 0.75 microgram/kg/week | | | | |
| Example 141 compound | 184.4 | 2.5 | 10.1 | 0.1 |
| 7.5 microgram/kg/week | | | | |

TABLE 6

| | Spinal | | | |
|---|---|---|---|---|
| | BMD | | serum Ca | |
| | mg/cm2 | S.E. | mg/dL | S.E. |
| SHAM | 189.5 | 3.9 | 10.7 | 0.1 |
| OVX | 176.6 | 4 | 10.2 | 0.1 |
| Alfacalcidol | 182.1 | 4.5 | 10.2 | 0.1 |
| 0.125 microgram/kg/week | | | | |
| Alfacalcidol | 180.9 | 3.7 | 10.6 | 0.1 |
| 0.25 microgram/kg/week | | | | |
| Alfacalcidol | 185.1 | 3.8 | 10.7 | 0.2 |
| 0.5 microgram/kg/week | | | | |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

We claim:

1. A compound of the formula: 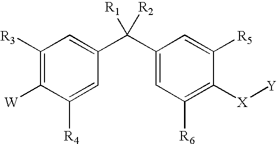
wherein
X is an optionally substituted methylene, an optionally substituted ethylene, an optionally substituted vinylene, an ethynylene, —S(O)n-, —NH—, or —O—;
n is an integer of 0 to 2;
Y is COOR8, CON(R9)R10, S(O)mR11 or a substituent represented by following formula: 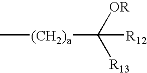
R8 and R11 are each independently selected from an optionally substituted C1-10 alkyl group or an optionally substituted C3-10 cycloalkyl group;
R9 and R10 are each independently selected from a hydrogen atom, an optionally substituted C1-10 alkyl group or an optionally substituted C3-10 cycloalkyl group;
m is an integer of 0 to 2;
a is an integer of 0 to 3;
R is a hydrogen atom or a protecting group for a hydroxyl group;
R12 and R13 are each independently selected from the group consisting of a hydrogen atom, an optionally substituted C1-10 alkyl group, an optionally substituted C3-10 cycloalkyl group, an optionally substituted C2-10 alkenyl group, an optionally substituted C2-10 alkynyl group, or R12 and R13 may together form an optionally substituted C3-C12 cycloalkyl group or an optionally substituted 3-12 membered heterocyclic group;
W is a hydroxyl group, a carboxyl group, a trifluoromethanesulfonyloxy group or a substituent represented by following formula: 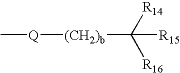
Q is —O—, —S—, —NH—, an optionally substituted methylene, an optionally substituted ethylene, an optionally substituted vinylene, an ethynylen, —(CH2)ₖ-NHC(═O)—, —(CH2)ₖ-C(═O)NH—, —(CH2)ₖ-NHC(═O)NH—, —O—(CH2)ₖ-NHC(═O)—, —O—(CH2)ₖ-C(═O)NH—, —O—(CH2)ₖ-NHC(═O)NH— or —(CH2)ₖ-SO₂—;
b is an integer of 0 to 10;
k is an integer of 0 to 2;
R14 is a hydrogen atom, a hydroxyl group, an optionally substituted carboxyl group, an optionally substituted carbamoyl group, an optionally substituted C1-6 alkyl group, an optionally substituted C3-C12 cycloalkyl group, an optionally substituted C1-6 alkenyl group, an optionally substituted C1-6 alkynyl group, an optionally substituted C6-C12 aryl group, an optionally substituted (C6-C12)aryl-(C1-4)alkyl group, —OR17 or —N(R18)R19;
R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group, or R15 and R16 may together form ═O;
R17 is selected from an optionally substituted C1-6 alkyl group or an optionally substituted C3-C6 cycloalkyl group;
R18 and R19 are each independently selected from the group consisting of a hydrogen atom, an optionally substituted C1-6 alkyl group, an optionally substituted C3-C6 cycloalkyl group, or R18 and R19 may together form an optionally substituted C3-C12 cycloalkyl group or an optionally substituted 3-12 membered heterocyclic group;
or one of (R15 and R17), (R16 and R17), (R15 and R18), (R16 and R18), (R15 and R19) or (R16 and R19) may together form a 3-12 membered cyclic ring which is selected from the group consisting of an amidine ring, an amine ring, an ether ring, a lactam ring, a lactone ring, an acetal ring, a hemiacetal ring, a carbonate ring, a carbamate ring, an urea ring, combinations thereof;
R1 and R2 are each independently selected from the group consisting of a C1-6 alkyl group optionally substituted with a halogen atom(s), a C3-6 cycloalkyl group optionally substituted with a halogen atom(s), a C2-6 alkenyl group optionally substituted with a halogen atom(s), a C2-6 alkynyl group optionally substituted with a halogen atom(s), or R1 and R2 may together form a C3-8 cycloalkyl group optionally substituted with a halogen atom(s), a C3-8 cycloalkenyl group optionally substituted with a halogen atom(s) or a C3-8 cycloalkylidene group optionally substituted with a halogen atom(s);
R3, R4, R5 and R6 are each independently selected from the group consisting of a hydrogen atom, a halogen atom, a C1-6 alkyl group optionally substituted with a halogen atom(s), a C3-6 cycloalkyl group optionally substituted with a halogen atom(s);
When X is —S(O)n-, —NH— or —O—, Q is selected from the group consisting of a methylene which may be substituted an C1-4 alkyl group, an ethylene, a vinylene, an ethynylene, —(CH2)ₖ-NHC(═O)—, —(CH2)ₖ-C(═O)NH—, —(CH2)ₖ-NHC(═O)NH—, —(CH2)ₖ-SO₂—;
When Q is —O—, —S—, —NH—, —O—(CH2)ₖ-NHC(═O)—, —O—(CH2)ₖ-C(═O)NH— or —O—(CH2)ₖ-NHC(═O)NH—, X is selected from the group consisting of an optionally substituted methylene, an optionally substituted ethylene, an optionally substituted vinylene, an ethynylene;
and pharmaceutically acceptable salts and prodrugs thereof.

2. The compound according to claim 1 wherein
X is an optionally substituted methylene, an optionally substituted ethylene, an optionally substituted vinylene or an ethynylene;
Y is COOR8, CON(R9)R10, S(O)mR11 or a substituent represented by following formula: 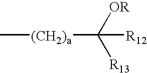
R is a hydrogen atom;
R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;
W is a substituent represented by following formula: 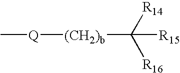
Q is —O—, a methylene, an ethylene, a vinylene, an ethynylene, —(CH2)ₖ-C(═O)NH— or —O—(CH2)ₖ-C(═O)NH—;
R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group selected from the group consisting of an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group, an isoquinoline group, a cinnoline group, a phthalazine group, a quinazoline group, a quinoxaline group, or R15 and R16 may together form ═O;
R17 is an optionally substituted C1-6 alkyl group or an optionally substituted C3-C6 cycloalkyl group;
R18 and R19 are each independently selected from a hydrogen atom, an optionally substituted C1-6 alkyl group or an optionally substituted C3-C6 cycloalkyl group;
or one of (R15 and R17), (R16 and R17), (R15 and R18), (R16 and R18), (R15 and R19) or (R16 and R19) may together form a 3-12 membered cyclic ring which is selected from a lactam ring or a lactone ring;
R1 is a C1-6 alkyl group;
R2 is a C1-6 alkyl group;
R3 is a hydrogen atom or a C1-6 alkyl group;
R4 is a halogen atom or a C1-6 alkyl group;
R5 is a hydrogen atom;
R6 is a halogen atom or a C1-6 alkyl group.

3. The compound according to claim 2 wherein
X is an ethylene, a vinylene, or an ethynylene;
Y is a substituent represented by following formula: 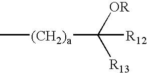
R is a hydrogen atom;
R12 and R13 are each independently selected from the group consisting of a hydrogen atom, a C1-8 alkyl group which may be substituted with a halogen atom(s), a C3-8 cycloalkyl group which may be substituted with a C1-4 alkyl group, or R12 and R13 are together form a C3-8 cycloalkyl group which may be substituted with a C1-4 alkyl group;
W is a substituent represented by following formula: 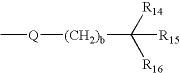
Q is —O—, a methylene, an ethylene, a vinylene, an ethynylene, —(CH2)ₖ-C(═O)NH— or —O—(CH2)ₖ-C(═O)NH—;
b is an integer of 0 to 5;
R14 is a hydrogen atom, a hydroxyl group, a carboxyl group which may be substituted with a C1-4 alkyl group, a carbamoyl group which may be substituted with a C1-4 alkyl group, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, —OR17 or —N(R18)R19;
R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, a 3-8 membered heterocyclic group which may be substituted with a C1-4 alkyl group, a hydroxyl group, a carboxyl group, a carbamoyl group or an amino group, wherein said heterocyclic group selected from the group consisting of an oxetane group, a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a pyridine group, a pyridazine group, a pyrimidine group, a pyrazine group, a tetrahydropyran group, a pyran group, a thiopyran group, or R15 and R16 may together form ═O;
and at least one of R14, R15 or R16 is a hydrogen atom;
R17 is a C1-4 alkyl group;
R18 and R19 are each independently selected from a hydrogen atom or a C1-4 alkyl group;
or one of (R15 and R17), (R16 and R17), (R15 and R18), (R16 and R18), (R15 and R19) or (R16 and R19) may together form a 3-12 membered lactone ring.

4. The compound according to claim 3 wherein
R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;
W is a substituent represented by following formula: 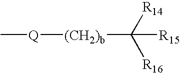
Q is —O—, —(CH2)ₖ-C(═O)NH— or —O—(CH2)ₖ-C(═O)NH—;
b is0, 1 or2;
k is 1;
R14 is a hydrogen atom, a hydroxyl group, a carboxyl group, a carbamoyl group, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, —OR17 or —N(R18)R19;
R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a carboxyl group, a 3-8 membered heterocyclic group selected from the group consisting of a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a pyridine group, a tetrahydropyran group;
and at least one of R14, R15 or R16 is a hydrogen atom;
R18 is a hydrogen atom;
R19 is a hydrogen atom;
or one of (R15 and R17), (R16 and R17), (R15 and R18), (R16 and R18), (R15 and R19) or (R16 and R19) may together form a 3-12 membered lactone ring;
R1 is a C1-4 alkyl group;
R2 is a C1-4 alkyl group;
R3 is a hydrogen atom or a C1-4 alkyl group;
R4 is a halogen atom or a C1-4 alkyl group;
R5 is a hydrogen atom;
R6 is a halogen atom or a C1-4 alkyl group.

5. The compound according to claim 4 wherein
R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),
or R12 and R13 are together form a C3-10 cycloalkyl group;
Q is —O— or —O—(CH2)ₖ-C(═O)NH—;
R1 is an ethyl group;
R2 is an ethyl group;
R3 is a hydrogen atom or a methyl group;
R4 is a chlorine atom or a methyl group;
R6 is a chlorine atom, a methyl group, an ethyl group, a propyl group or an isopropyl group.

6. The compound according to claim 5 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl )-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{I -Ethyl-1-[3-methyl-4-((E)-4,4,4-trifluoro-3-hydroxy-3-trifluoromethyl-but-1-enyl )-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol, (R)-5-(2-Chloro-4-{1-[3-chloro-4-((R)-3-hydroxy-4,4-dimethyl-pentyl)-phenyl]-1-ethyl-propyl}-phenoxymethyl)-dihydro-furan-2-one, (R)-5-(2-Chloro-4-{1-[3-chloro-4-((S)-3-hydroxy-4,4-dimethyl-pentyl)-phenyl]-1-ethyl-propyl}-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[4-(3-ethyl-3-hydroxy-pent-1-ynyl)-3-methyl-phenyl]-propyl}-2-methyl-phenoxymethyl)-dihydro-furan-2-one, (R)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (R)-5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclohexyl)-propyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopentyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopentyl)-propyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-propyl]-3-methyl-phenyl}-propyl)-2-methyl-phenoxymethyl]-dihydro-furan-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (R)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one, (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one, (S)-5-(4-{1-Ethyl-1-[3-methyl-4-(2-methyl-propane-2-sulfinylmethyl)-phenyl]-propyl}-2-methyl-phenoxymethyl)-dihydro-furan-2-one, (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one, (S)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one, (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-non-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-en-4-ynyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-methyl-but-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-propyl-hex-1-enyl )-3-methyl-phenyl]-propyl}-2-methyl-phenoxymethyl)-dihydro-furan-2-one, (S)-5-(4-{1-Ethyl-1-[4-((E)-3-hydroxy-4,4-dimethyl-pent-1enyl)-3-methyl-phenyl]-propyl}-2-methyl-phenoxymethyl)-dihydro-furan-2-one, (S)-5-(4-{1-Ethyl-1-[4-(1-hydroxy-cyclobutylethynyl)-3-methyl-phenyl]-propyl}-2-methyl-phenoxymethyl)-dihydro-furan-2-one, (S )-5-(4-{1-Ethyl-1-[4-(1-hydroxy-cyclohexylethynyl)-3-methyl-phenyl]-propyl}-2-methyl-phenoxymethyl)-dihydro-furan-2-one, (S)-5-(4-{1-Ethyl-1-[4-(1-hydroxy-cyclopentylethynyl)-3-methyl-phenyl]-propyl}-2-methyl-phenoxymethyl)-dihydro-furan-2-one, (S)-5-(4-{1-Ethyl-1-[4-(3-ethyl-3-hydroxy-pentyl)-3-methyl-phenyl]-propyl}-2-methyl-phenoxymethyl)-dihydro-furan-2-one, (S)-5-(4-{1-Ethyl-1-[4-(3-hydroxy-3-methyl-but-1-ynyl)-3-methyl-phenyl]-propyl}-2-methyl-phenoxymethyl)-dihydro-furan-2-one, (S)-5-(4-{1-Ethyl-1-[4-(3-hydroxy-3-propyl-hex-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-decyl)-3-methyl-phenyl]-propyl}-2-methyl-phenoxymethyl)-dihydro-furan-2-one, (S )-5-(4-{1-Ethyl-1-[4-(3-hydroxy-4,4-dimethyl-heptyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-nonyl)-3-methyl-phenyl]-propyl}-2-methyl-phenoxymethyl)-dihydro-furan-2-one, (S)-5-(4-{l-Ethyl-1-[4-(3-hydroxy-4,4-dimethyl-octyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pent-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (S)-5-[4-(1-Ethyl-1-{4-[(E)-2-(1-hydroxy-cyclobutyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclohexyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclopentyl)-vinyl]-3-methyl-phenyl}-propyl)-2-methyl-phenoxymethyl]-dihydro-furan-2-one, (S)-5-{4-[1-(4-tert-Butylsulfanylmethyl-3-methyl-phenyl)-1-ethyl-propyl]-2-methyl-phenoxymethyl}-dihydro-furan-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[4-((R)-3-hydroxy-4,4-dimethyl-pentyl)-3-methyl-phenyl]-propyl}-2-methyl-phenoxymethyl)-tetrahydro-pyran-2-one, (S)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one.

7. The compound according to claim 5 wherein R3 is a hydrogen atom.

8. The compound according to claim 7 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

9. The compound according to claim 5 wherein R4 is a chlorine atom.

10. The compound according to claim 9 selected from 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt.

11. The compound according to claim 5 wherein R6 is a chlorine atom.

12. The compound according to claim 11 selected from the group consisting of 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt.

13. The compound according to claim 5 wherein R6 is a methyl group.

14. The compound according to claim 13 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl)-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl )-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid.

15. The compound according to claim 5 wherein X is an ethylene.

16. The compound according to claim 15 selected from the group consisting of (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 1-(4-{1-[4-(5-Amino-pentyloxy)-3-methyl-phenyl]-1-ethyl-propyl}-2-methyl-phenyl)-4,4-dimethyl-pentan-3-ol, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

17. The compound according to claim 5 wherein X is a vinylene.

18. The compound according to claim 17 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S )-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid.

19. The compound according to claim 5 wherein X is an ethynylene.

20. The compound according to claim 19 selected from the group consisting of (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol.

21. The compound according to claim 5 wherein one of R12 and R13 is a hydrogen atom and the other is a C1-6 alkyl group.

22. The compound according to claim 21 wherein one of R12 and R13 is a hydrogen atom and the other is a tert-butyl group.

23. The compound according to claim 22 selected from the group consisting of (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 1-(4-{1-[4-(5-Amino-pentyloxy)-3-methyl-phenyl]-1-ethyl-propyl}-2-methyl-phenyl)-4,4-dimethyl-pentan-3-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid.

24. The compound according to claim 5 wherein both of R12 and R13 are same and a C1-6 alkyl group which may be substituted with a halogen atom(s).

25. The compound according to claim 24 wherein R12 is a trifluoromethyl group and R13 are is a trifluoromethyl group.

26. The compound according to claim 25 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl)-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E )-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4,-trifluoro-3-hydroxy-3-trifluoromethyl-but-1-enyl)-phenyl]-propyl}-2-methyl-phenoxy)-hexanoic acid, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl )-1,1,1-trifluoro-2-trifluoromethyl-but-3-en-2-ol, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-l -(tetrahydro-furan-2-yl)methoxy]-phenyl}-propyl)-2-methyl-phenyl]-1,1,1-trifluoro-2-trifluoromethyl-but-3-en-2-ol , (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl )-2-methyl-phenyl]-1,1,1-trifluoro-2-trifluoromethyl-but-3-en-2-ol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{-Ethyl-1-[4-((E)-3-ethyl-3-hydroxy-pent-1-enyl)-3-propyl-phenyl]-propyl}-2-methyl-phenoxy)-4-hydroxy-pentanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

27. The compound according to claim 5 wherein Q is —O—.

28. The compound according to claim 27 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{l-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl )-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-I -[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(R)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (E)-4-[4-(1-Ethyl-1-{3-methyl-4-[(S)-1-(tetrahydro-furan-2-yl)methoxy]-phenyl}-propyl)-2-methyl-phenyl]-1,1,1-trifluoro-2-trifluoromethyl-but-3-en-2-ol, (S)-5-(4-{1-Ethyl-I -[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 6(R)-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

29. The compound according to claim 5 wherein at least one of R14, R15 or R16 is a substituent which have a hydroxyl group, a carboxyl group, a carbamoyl group or an amino group.

30. The compound according to claim 29 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S )-5-(4-{1-Ethyl-1-[3-methyl-4-(4,4,4-trifluoro-3-hydroxy-3-trifluoromethyl-butyl)-phenyl]-propyl)-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 1-(4-{1-[4-(5-Amino-pentyloxy)-3-methyl-phenyl]-1-ethyl-propyl}-2-methyl-phenyl)-4,4-dimethyl-pentan-3-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (E)-1-(4-{1-[4-(5-Amino-pentyloxy)-3-methyl-phenyl]-1-ethyl-propyl}-2-methyl-phenyl)-3-ethyl-pent-1-en-3-ol, 1-[(E)-2-(4-{1-[4-(5-Amino-pentyloxy)-3-methyl-phenyl]-1-ethyl-propyl}-2-methyl-phenyl)-vinyl]-cyclopentanol, (E)-4-(4-{1-[4-(5-Amino-pentyloxy)-3-methyl-phenyl]-1-ethyl-propyl}-2-methyl-phenyl)-1,1,1,-trifluoro-2-trifluoromethyl-but-3-en-2-ol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 4-(4-{1-Ethyl-1-[4-((E)-3-ethyl-3-hydroxy-pent-1-enyl)-3-methyl-phenyl]-propyl}-2-methyl-phenoxymethyl)-benzoic acid, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-hydroxy-3-trifluoromethyl-but-1-enyl)-phenyl]-propyl}-2-methyl-phenoxy)-pentane-1,4-diol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, (E)-N-(2-Amino-ethyl)-2-(4-{1-ethyl-1-[4-(3-ethyl-3-hydroxy-pent-1-enyl)-3-methyl-phenyl]-propyl}-2-methyl-phenoxy)-acetamide, (E)-5-(4-{1-Ethyl-1-[4-(3-ethyl-3-hydroxy-pent-1-enyl)-3-methyl-phenyl]-propyl}-2-methyl-phenoxy)-4-oxo-pentanoic acid, 2-(R)-[2-(4-{1-Ethyl-1-[4-(3-hydroxy-4,4-dimethyl-pentyl)-3-methyl-phenyl]-propyl}-2-methyl-phenoxy)-acetylamino]-propionic acid, 2-(S)-[2-(4-{1-Ethyl-1-[4-(3-hydroxy-4,4-dimethyl-pentyl)-3-methyl-phenyl]-propyl}-2-methyl-phenoxy)-acetylamino]-3-phenyl-propionic acid, 5-(4-{1-Ethyl-1-[4-(3-hydroxy-4,4-dimethyl-pentyl)-3-methyl-phenyl]-propyl}-2-methyl-phenoxy)-4-oxo-pentanoic acid, 5-(4-{1-Ethyl-1-[4-(3-hydroxy4,4-dimethyl-pentyl)-3-methyl-phenyl]-propyl}-2-methyl-phenoxy)-pentane-1,4-diol.

31. The compound according to claim 5 wherein R14 is a hydroxyl group.

32. The compound according to claim 31 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-(1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, (S)-3-(4-{1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentane-1,4-diol, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentane-1,4-diol, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid, 5-(4-{1-Ethyl-1-[4-(3-hydroxy-4,4-dimethyl-pentyl)-3-methyl-phenyl]-propyl}-2-methyl-phenoxy)-pentane-1,4-diol.

33. The compound according to claim 5 wherein one of R15 and R16 is a hydrogen atom and the other is a C1-6 alkyl group substituted with a carboxyl group.

34. The compound according to claim 33 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{l -Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (R)-4-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-3-hydroxy-butyric acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 5-(4-{1-Ethyl-1-[4-(3-hydroxy-4,4-dimethyl-pentyl)-3-methyl-phenyl]-propyl}-2-methyl-phenoxy)-pentanoic acid, 5-(4-{1-Ethyl-1-[4-((E)-3-ethyl-3-hydroxy-pent-1-enyl)-3-methyl-phenyl]-propyl}-2-methyl-phenoxy)-pentanoic acid, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 5-[4-(1-Ethyl-1-{4-[(E)-2-(1-hydroxy-cyclohexyl)-vinyl]-3-methyl-phenyl}-propyl}-2-methyl-phenoxy]-pentanoic acid, 6-(4-{1-Ethyl-I -[3-methyl-4-((E)-4,4,4-trifluoro-3-hydroxy-3-trifluoromethyl-but-1-enyl)-phenyl]-propyl}-2-methyl-phenoxy)-hexanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid, 5-(4-{1-Ethyl-1-[4-(3-hydroxy-4,4-dimethyl-pentyl)-3-methyl-phenyl]-propyl}-2-methyl-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid, 5-(2-Chloro-4-{1-[3-chloro-4-(3-hydroxy-4,4-dimethyl-pentyl)-phenyl]-1-ethyl-propyl}-phenoxy)-4(R)-hydroxy-pentanoic acid sodium salt, (S)-5-(4-{1-Ethyl-1-[4-((E)-3-ethyl-3-hydroxy-pent-1-enyl)-3-methyl-phenyl]-propyl}-phenoxy)-4-hydroxy-pentanoic acid, (S)-5-(4-{1-Ethyl-1-[4-((E)-3-ethyl-3-hydroxy-pent-1-enyl)-3-methyl-phenyl]-propyl}-2,6-dimethyl-phenoxy)-4-hydroxy-pentanoic acid, (S)-5-(4-{1-Ethyl-1-[4-((E)-3-ethyl-3-hydroxy-pent-1-enyl)-3-propyl-phenyl]-propyl}-2-methyl-phenoxy)-4-hydroxy-pentanoic acid, 6-(4-{1-Ethyl-1-[4-(3-hydroxy-4,4-dimethyl-pentyl)-3-methyl-phenyl]-propyl}-2-methyl-phenoxy)-5(S)-hydroxy-hexanoic acid.

35. The compound according to claim 5 wherein one of (R15 and R17), (R16 and R17), (R15 and R18), (R16 and R18), (R15 and R19) or (R16 and R19) together form a 3-12 membered lactone ring.

36. The compound according to claim 35 selected from the group consisting of (R)-5-(2-Chloro-4-{1-[3-chloro-4-((R)-3-hydroxy-4,4-dimethyl-pentyl)-phenyl]-1-ethyl-propyl}-phenoxymethyl)-dihydro-furan-2-one, (R)-5-(2-Chloro-4-{1-[3-chloro-4-((S)-3-hydroxy-4,4-dimethyl-pentyl)-phenyl]-1-ethyl-propyl}-phenoxymethyl)-dihydro-furan-2-one, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (R)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (R)-5-(4-{1-Ethyl-1-[4-(3-ethyl-3-hydroxy-pent-1-ynyl)-1-methyl-phenyl]-propyl}-2-methyl-phenoxymethyl)-dihydro-furan-2-one, (R)-5-(4-{1-Ethyl-1-[4-(3-hydroxy-4,4-dimethyl-pentyl)-3-methyl-phenyl]-propyl}-2-methyl-phenoxymethyl)-dihydro-furan-2-one, (R)-5-[4-(1-Ethyl-1-{4-[3-hydroxy-3-(1-methyl-cyclohexyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclohexyl)-propyl]-3-methyl-phenyl}-propyl )-2-methyl-phenoxymethyl]-dihydro-furan-2-one, (R)-5-[4-(1-Ethyl-1-{4-[3-hydroxy-3-(1-methyl-cyclopentyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopentyl)-propyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-prop-1-ynyl]-3-methyl-phenyl}-propyl)-2-methyl-phenoxymethyl]-dihydro-furan-2-one, (R)-5-[4-(1-Ethyl-1-{4-[3-hydroxy-3-(1-methyl-cyclopropyl)-propyl]-3-methyl-phenyl}-propyl)-2-methyl-phenoxymethyl]-dihydro-furan-2-one, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (R)-6-(4-{1-Ethyl-1-[4-((E)-3-ethyl-3-hydroxy-pent-1-enyl)-3-methyl-phenyl]-propyl}-2-methyl-phenoxymethyl)-tetrahydro-pyran-2-one, (R)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one, (S)-5-(4-{1-[4-((E)-1,3-Diethyl-3-hydroxy-pent-1-enyl)-3-methyl-phenyl]-1-ethyl-propyl}-2-methyl-phenoxymethyl)-dihydro-furan-2-one, (S )-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-dihydro-furan-2-one, (S)-5-(4-{1-Ethyl-1-[3-methyl-4-((E)-4,4,5,5,5-pentafluoro-3-hydroxy-3-pentafluoroethyl-pent-1-enyl)-phenyl]-propyl}-2-methyl-phenoxymethyl)-dihydro-furan-2-one, (S)-5-(4-{1-Ethyl-1-[3-methyl-4-(2-methyl-propane-2-sulfinylmethyl)-phenyl]-propyl}-2-methyl-phenoxymethyl)-dihydro-furan-2-one, (S)-5-(4-{1-Ethyl-1-[3-methyl-4-(2-methyl-propane-2-sulfonylmethyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-dihydro-furan-2-one, (S)-5-(4-{1-Ethyl-1-[3-methyl-4-(4,4,4-trifluoro-3-hydroxy-3-trifluoromethyl-butyl)-phenyl]-propyl}-2-methyl-phenoxymethyl)-dihydro-furan-2-one, (S)-5-(4-{1-Ethyl-1-[3-methyl-4-(4,4,4-trifluoro-3-methoxymethoxy-3-trifluoromethyl-but-1-ynyl)-phenyl]-propyl}-2-methyl-phenoxymethyl)-dihydro-furan-2-one, (S)-5-(4-{1-Ethyl-1-[4-((E)-3-ethyl-3-hydroxy-1-methyl-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-dec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hept-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-hex-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-non-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-en-4-ynyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-oct-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-pent-1-enyl)-3-methyl-phenyl]-propyl}-phenoxymethyl)-dihydro-furan-2-one, (S)-5-(4-{1-Ethyl-1-[4-((E)-3-ethyl-3-hydroxy-pent-1-enyl)-3-propyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-ethyl-3-hydroxy-undec-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-methyl-but-1-enyl)-3-methyl-phenyl]-propyl}-2-methyl-phenoxymethyl)-dihydro-furan-2-one, (S)-5-(4-{1-Ethyl-1-[4-((E)-3-hydroxy-3-propyl-hex-1-enyl)-3-methyl-phenyl]-propyl}-2-methyl-phenoxymethyl)-dihydro-furan-2-one, (S )-5-(4-{1-Ethyl-1-[4-((E)-3-hydroxy-4,4-dimethyl-pent-1-enyl)-3-methyl-phenyl]-propyl}-2-methyl-phenoxymethyl )-dihydro-furan-2-one, (S)-5-(4-{1-Ethyl-1-[4-(1-hydroxy-cyclobutylethynyl)-3-methyl-phenyl]-propyl}-2-methyl-phenoxymethyl)-dihydro-furan-2-one, (S)-5-(4-{1-Ethyl-1-[4-(1-hydroxy-cyclohexylethynyl)-3-methyl-phenyl]-propyl)-2-methyl-phenoxymethyl)-dihydro-furan-2-one, (S)-5-(4-{1-Ethyl-1-[4-(1-hydroxy-cyclopentylethynyl)-3-methyl-phenyl]-propyl}-2-methyl-phenoxymethyl)-dihydro-furan-2-one, (S)-5-(4-{1-Ethyl-1-[4-(3-ethyl-3-hydroxy-pentyl)-3-methyl-phenyl]-propyl}-2-methyl-phenoxymethyl)-dihydro-furan-2-one, (S)-5-(4-{1-Ethyl-1-[4-(3-hydroxy-3-methyl-but-1-ynyl)-3-methyl-phenyl]-propyl}-2-methyl-phenoxymethyl)-dihydro-furan-2-one, (S)-5-(4-{1-Ethyl-1-[4-(3-hydroxy-3-propyl-hex-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-decyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-heptyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-nonyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-octyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pent-1-ynyl)-3-methyl-phenyl]-propyl}-2-methyl-phenoxymethyl)-dihydro-furan-2-one, (S)-5-(4-{1-Ethyl-1-[4-(3-hydroxy-4,4-dimethyl-pentyl)-3-methyl-phenyl]-propyl}-2-methyl-phenoxymethyl )-dihydro-furan-2-one, (S)-5-[4-(1-Ethyl-1-{4-[(E)-2-(1-hydroxy-cyclobutyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclohexyl)-vinyl]-3-methyl-phenyl}-propyl)-2-methyl-phenoxymethyl]-dihydro-furan-2-one, (S)-5-[4-(1-Ethyl-1-{4-[(E)-2-(1-hydroxy-cyclopentyl)-vinyl]-3-methyl-phenyl}-propyl)-2-methyl-phenoxymethyl]-dihydro-furan-2-one, (S)-5-{4-[1-(4-tert-Butylsulfanylmethyl-3-methyl-phenyl)-1-ethyl-propyl]-2-methyl-phenoxymethyl}-dihydro-furan-2-one, (S)-6-(4-{1-Ethyl-1-[3-methyl-4-((E)-4,4,4-trifluoro-3-hydroxy-3-trifluoromethyl-but-1-enyl )-phenyl]-propyl}-2-methyl-phenoxymethyl)-tetrahydro-pyran-2-one, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-tetrahydro-pyran-2-one, (S)-6-(4-{1-Ethyl-1-[4-((R)-3-hydroxy-4,4-dimethyl-pentyl)-3-methyl-phenyl]-propyl}-2-methyl-phenoxymethyl)-tetrahydro-pyran-2-one, (S)-6-(4-{1-Ethyl-1-[4-((S)-3-hydroxy-4,4-dimethyl-pentyl)-3-methyl-phenyl]-propyl}-2-methyl-phenoxymethyl)-tetrahydro-pyran-2-one.

37. The compound according to claim 4 wherein
R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),
or R12 and R13 are together form a C3-10 cycloalkyl group;
Q is a methylene, an ethylene, an ethynylene or —(CH2)ₖ-C(═O)NH—;
R1 is an ethyl group;
R2 is an ethyl group;
R3 is a hydrogen atom;
R4 is a chlorine atom or a methyl group;
R6 is a chlorine atom or a methyl group.

38. The compound according to claim 37 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

39. The compound according to claim 37 wherein R3 is a hydrogen atom.

40. The compound according to claim 39 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

41. The compound according to claim 37 wherein R4 is a chlorine atom.

42. The compound according to claim 37 wherein R6 is a chlorine atom.

43. The compound according to claim 37 wherein R6 is a methyl group.

44. The compound according to claim 43 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

45. The compound according to claim 37 wherein X is an ethylene.

46. The compound according to claim 45 which is 6-(4-{1-Ethyl-1-[4-(3-hydroxy-4,4-dimethyl-pentyl)-3-methyl-phenyl]-propyl}-2-methyl-phenyl)-hex-5-ynoic acid.

47. The compound according to claim 37 wherein X is a vinylene.

48. The compound according to claim 47 selected from the group consisting of 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-4(4,4,4-trifluoro-3-hydroxy-3-trifluoromethyl-(E)-but-1-enyl)-phenyl]-propyl}-2-methyl-phenyl)-hept-6-ynoic acid.

49. The compound according to claim 37 wherein X is an ethynylene.

50. The compound according to claim 49 selected from 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid or 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid.

51. The compound according to claim 37 wherein one of R12 and R13 is a hydrogen atom and the other is a C1-6 alkyl group.

52. The compound according to claim 51 wherein one of R12 and R13 is a hydrogen atom and the other is a tert-butyl group.

53. The compound according to claim 52 which is 6-(4-{1-Ethyl-1-[4-(3-hydroxy-4,4-dimethyl-pentyl)-3-methyl-phenyl]-propyl}-2-methyl-phenyl)-hex-5-ynoic acid.

54. The compound according to claim 37 wherein both of R12 and R13 are same and a C1-6 alkyl group which may be substituted with a halogen atom(s).

55. The compound according to claim 54 wherein R12 is a trifluoromethyl group and R13 are is a trifluoromethyl group.

56. The compound according to claim 55 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid.

57. The compound according to claim 37 wherein Q is an ethylene.

58. The compound according to claim 57 selected from 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid or 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid.

59. The compound according to claim 37 wherein Q is an ethynylene.

60. The compound according to claim 59 selected from the group consisting of 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 7-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

61. The compound according to claim 37 wherein at least one of R14, R15 or R16 is a substituent which have a hydroxyl group, a carboxyl group, a carbamoyl group or an amino group.

62. The compound according to claim 61 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 7-(4-{1-Ethyl-i-[3-methyl-4-(4,4,4-trifluoro-3-hydroxy-3-trifluoromethyl-but-1-ynyl)-phenyl]-propyl}-2-methyl-phenyl )-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

63. The compound according to claim 37 wherein R14 is a hydroxyl group.

64. The compound according to claim 37 wherein one of R15 and R16 is a hydrogen atom and the other is a C1-6 alkyl group substituted with a carboxyl group.

65. The compound according to claim 64 selected from the group consisting of 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenyl)-hex-5-ynoic acid, 6-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hex-5-ynoic acid, 5-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-pent-4-ynoic acid, 7-(4-{1-Ethyl-1-[3-methyl-4-(4,4,4-trifluoro-3-hydroxy-3-trifluoromethyl-(E)-but-1-enyl)-phenyl]-propyl}-2-methyl-phenyl)-hept-6-ynoic acid, 6-(4-{1-Ethyl-1-[4-(3-hydroxy-4,4-dimethyl-pentyl)-3-methyl-phenyl]-propyl}-2-methyl-phenyl)-hex-5-ynoic acid.

66. The compound according to claim 37 wherein one of (R15 and R17), (R16 and R17), (R15 and R18), (R16 and R18), (R15 and R19) or (R16 and R19) together form a 3-12 membered lactone ring.

67. The compound according to claim 1 wherein X is an optionally substituted vinylene, or an ethynylene.

68. The compound according to claim 1 wherein
W is a substituent represented by following formula: 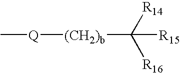
Q is selected from the group consisting of —O—, a methylene, an ethylene, a vinylene, and an ethynylene; and wherein
b is 1;
R14 is a hydroxyl group;
R15 is a hydrogen atom;
R16 is a C1-6 alkyl group substituted with a carboxyl group.

69. The compound according to claim 1 selected from the group consisting of (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-5-(4-{1-Ethyl-1-[4-(3-ethyl-3-hydroxy-pent-1-ynyl)-3-methyl-phenyl]-propyl}-2-methyl-phenoxy)-4-hydroxy-pentanoic acid, (R)-5-(4-{1-Ethyl-1-[4-(3-ethyl-3-hydroxy-pent-1-ynyl)-3-methyl-phenyl]-propyl}-2-methyl-phenoxy)-4-hydroxy-pentanoic acid, (S)-6-(4-{1-Ethyl-1-[4-(3-ethyl-3-hydroxy-pent-1-ynyl)-3-methyl-phenyl]-propyl}-2-methyl-phenoxy)-5-hydroxy-hexanoic acid, (R)-6-(4-{1-Ethyl-1-[4-(3-ethyl-3-hydroxy-pent-1-ynyl)-3-methyl-phenyl]-propyl}-2-methyl-phenoxy)-5-hydroxy-hexanoic acid, (S)-3-(4-{1-Ethyl-1-[4-(3-ethyl-3-hydroxy-pent-1-ynyl)-3-methyl-phenyl]-propyl}-2-methyl-phenoxy)-propane-1,2-diol, 2-(4-{1-Ethyl-1-[4-(3-ethyl-3-hydroxy-pent-1-ynyl)-3-methyl-phenyl]-propyl}-2-methyl-phenoxymethyl)-propane-1,3-diol, 4-{1-Ethyl-1-[4-(3-ethyl-3-hydroxy-pent-1-ynyl)-3-methyl-phenyl]-propyl}-2-methyl-phenol, 5-(4-{1-Ethyl-1-[4-(3-ethyl-3-hydroxy-pent-1-ynyl)-3-methyl-phenyl]-propyl}-2-methyl-phenoxy)-pentanoic acid, 6-(4-{1-Ethyl-1-[4-(3-ethyl-3-hydroxy-pent-1-ynyl)-3-methyl-phenyl]-propyl}-2-methyl-phenoxy)-hexanoic acid, 3-(4-{1-Ethyl-1-[4-(3-ethyl-3-hydroxy-pent-1-ynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-3-ethyl-pent-1-yn-3-ol, 3-Ethyl-1-[4-(1-ethyl-1-{3-methyl-4-[4-(1H-tetrazol-5-yl)-butoxy]-phenyl}-propyl)-2-methyl-phenyl]-pent-1-yn-3-ol, (S)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (R)-5-(4-{1-Ethyl-1-[4-((E)-3-ethyl-3-hydroxy-pent-1-enyl)-3-methyl-phenyl]-propyl}-2-methyl-phenoxy)-4-hydroxy-pentanoic acid, (S)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid, (R)-6-(4-{1-Ethyl-1-[4-((E)-3-ethyl-3-hydroxy-pent-1-enyl)-3-methyl-phenyl]-propyl}-2-methyl-phenoxy)-5-hydroxy-hexanoic acid, (S)-3-(4-{1-Ethyl-1-[4-((E)-3-ethyl-3-hydroxy-pent-1-enyl)-3-methyl-phenyl]-propyl}-2-methyl-phenoxy)-propane-1,2-diol, 2-(4-{1-Ethyl-1-[4-((E)-3-ethyl-3-hydroxy-pent-1-enyl)-3-methyl-phenyl]-propyl}-2-methyl-phenoxymethyl)-propane-1,3-diol, 4-{1-Ethyl-1-[4-((E)-3-ethyl-3-hydroxy-pent-1-enyl)-3-methyl-phenyl]-propyl}-2-methyl-phenol, 5-(4-{1-Ethyl-1-[4-((E)-3-ethyl-3-hydroxy-pent-1-enyl)-3-methyl-phenyl]-propyl}-2-methyl-phenoxy)-pentanoic acid, 6-(4-{1-Ethyl-1-[4-((E)-3-ethyl-3-hydroxy-pent-1-enyl)-3-methyl-phenyl]-propyl}-2-methyl-phenoxy)-hexanoic acid, 3-(4-{1-Ethyl-1-[4-((E)-3-ethyl-3-hydroxy-pent-1-enyl)-3-methyl-phenyl]-propyl}-2-methyl-phenyl)-propionic acid, (E)-1-(4-{1-[4-(5-Amino-pentyloxy)-3-methyl-phenyl]-1-ethyl-propyl}-2-methyl-phenyl)-3-ethyl-pent-1-en-3-ol, (E)-3-Ethyl-1-[4-(1-ethyl-1-{3-methyl-4-[-4-(1H-tetrazol-5-yl)-butoxy]-phenyl}-propyl)-2-methyl-phenyl]-pent-1-en-3-ol, (S)-5-(4-{1-Ethyl-1-[4-(3-ethyl-3-hydroxy-pentyl)-3-methyl-phenyl]-propyl}-2-methyl-phenoxy)-4-hydroxy-pentanoic acid, (R)-5-(4-{1-Ethyl-1-[4-(3-ethyl-3-hydroxy-pentyl)-3-methyl-phenyl]-propyl}-2-methyl-phenoxy)-4-hydroxy-pentanoic acid, (S)-6-(4-{1-Ethyl-1-[4-(3-ethyl-3-hydroxy-pentyl)-3-methyl-phenyl]-propyl}-2-methyl-phenoxy)-5-hydroxy-hexanoic acid, (R)-6-(4-{1-Ethyl-1-[4-(3-ethyl-3-hydroxy-pentyl)-3-methyl-phenyl]-propyl}-2-methyl-phenoxy)-5-hydroxy-hexanoic acid, (S)-3-(4-{1-Ethyl-1-[4-(3-ethyl-3-hydroxy-pentyl)-3-methyl-phenyl]-propyl}-2-methyl-phenoxy)-propane-1,2-diol, 2-(4-{1-Ethyl-1-[4-(3-ethyl-3-hydroxy-pentyl )-3-methyl-phenyl]-propyl}-2-methyl-phenoxymethyl)-propane-1,3-diol, 4-{1-Ethyl-1-[4-(3-ethyl-3-hydroxy-pentyl)-3-methyl-phenyl]-propyl}-2-methyl-phenol, 5-(4-{1-Ethyl-1-[4-(3-ethyl-3-hydroxy-pentyl)-3-methyl-phenyl]-propyl}-2-methyl-phenoxy)-pentanoic acid, 6-(4-{1-Ethyl-1-[4-(3-ethyl-3-hydroxy-pentyl)-3-methyl-phenyl]-propyl}-2-methyl-phenoxy)-hexanoic acid, 3-(4-{1-Ethyl-1-[4-(3-ethyl-3-hydroxy-pentyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-3-ethyl-pentan-3-ol, 3-Ethyl-1-[4-(1-ethyl-1-{3-methyl-4-[4-(1H-tetrazol-5-yl)-butoxy]-phenyl}-propyl)-2-methyl-phenyl]-pentan-3-ol, (R)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid, (S)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-5-hydroxy-hexanoic acid, (S)-3-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-but-1-ynyl)-phenyl]-propyl}-2-methyl-phenoxymethyl)-propane-1,3-diol, 4-{1-Ethyl-1-[3-methyl-4-(4,4,4-trifluoro-3-hydroxy-3-trifluoromethyl-but-1-ynyl)-phenyl]-propyl}-2-methyl-phenol, 5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-pentanoic acid, 6-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-hexanoic acid, 3-(4-{1-Ethyl-1-[3-methyl-4-(4,4,4-trifluoro-3-hydroxy-3-trifluoromethyl-but-1-ynyl)-phenyl]-propyl}-2-methyl-phenyl)-propionic acid, 4-(4-{1-[4-(5-Amino-pentyloxy)-3-methyl-phenyl]-1-ethyl-propyl}-2-methyl-phenyl)-1,1,1-trifluoro-2-trifluoromethyl-but-3-yn-2-ol, 4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-yn-2-ol, (S)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (R)-5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-4-hydroxy-pentanoic acid, (S)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (R)-6-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-5-hydroxy-hexanoic acid, (S)-3-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-propane-1,2-diol, 2-(4-{1-Ethyl-1-[3-methyl-4-((E)-4,4,4-trifluoro-3-hydroxy-3-trifluoromethyl-but-1-enyl)-phenyl]-propyl}-2-methyl-phenoxymethyl)-propane-1,3-diol, 4-{1-Ethyl-1-[3-methyl-4-((E)-4,4,4-trifluoro-3-hydroxy-3-trifluoromethyl-but-1-enyl)-phenyl]-propyl}-2-methyl-phenol, 5-(4-{1-Ethyl-1-[3-methyl-4-((E)-4,4,4-trifluoro-3-hydroxy-3-trifluoromethyl-but-1-enyl)-phenyl]-propyl}-2-methyl-phenoxy)-pentanoic acid, 6-(4-({-Ethyl-1-[3-methyl-4-((E)-4,4,4-trifluoro-3-hydroxy-3-trifluoromethyl-but-1-enyl)-phenyl]-propyl}-2-methyl-phenoxy)-hexanoic acid, 3-(4-{1-Ethyl-1-[3-methyl-4-((E)-4,4,4-trifluoro-3-hydroxy-3-trifluoromethyl-but-1-enyl)-phenyl]-propyl}-2-methyl-phenyl)-propionic acid, (E)-4-(4-{1-[4-(5-Amino-pentyloxy)-3-methyl-phenyl]-1-ethyl-propyl}-2-methyl-phenyl)-1,1,1-trifluoro-2-trifluoromethyl-but-3-en-2-ol, (E)-4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-but-3-en-2-ol, (S)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (R)-5-(4-{1-Ethyl-1-[3-methyl-4-(4,4,4-trifluoro-3-hydroxy-3-trifluoromethyl-butyl)-phenyl]-propyl}-2-methyl-phenoxy)-4-hydroxy-pentanoic acid, (S)-6-(4-{1-Ethyl-1-[3-methyl-4-(4,4,4-trifluoro-3-hydroxy-3-trifluoromethyl-butyl)-phenyl]-propyl}-2-methyl-phenoxy)-5-hydroxy-hexanoic acid, (R)-6-(4-{1-Ethyl-1-[3-methyl-4-(4,4,4-trifluoro-3-hydroxy-3-trifluoromethyl-butyl)-phenyl]-propyl}-2-methyl-phenoxy)-5-hydroxy-hexanoic acid, (S)-3-(4-(1-Ethyl-1-[3-methyl-4-(4,4,4-trifluoro-3-hydroxy-3-trifluoromethyl-butyl)-phenyl]-propyl}-2-methyl-phenoxy)-propane-1,2-diol, 2-(4-{1-Ethyl-1-[3-methyl-4-(4,4,4-trifluoro-3-hydroxy-3-trifluoromethyl-butyl)-phenyl]-propyl}-2-methyl-phenoxymethyl)-propane-1,3-diol, 4-{1-Ethyl-1-[3-methyl-4-(4,4,4-trifluoro-3-hydroxy-3-trifluoromethyl-butyl)-phenyl]-propyl}-2-methyl-phenol, 5-(4-{1-Ethyl-1-[3-methyl-4-(4,4,4-trifluoro-3-hydroxy-3-trifluoromethyl-butyl)-phenyl]-propyl}-2-methyl-phenoxy)-pentanoic acid, 6-(4-{1-Ethyl-1-[3-methyl-4-(4,4,4-trifluoro-3-hydroxy-3-trifluoromethyl-butyl)-phenyl]-propyl}-2-methyl-phenoxy)-hexanoic acid, 3-(4-{1-Ethyl-1-[3-methyl-4-(4,4,4-trifluoro-3-hydroxy-3-trifluoromethyl-butyl)-phenyl]-propyl}-2-methyl-phenyl)-propionic acid, 4-(4-{(-[4-(5-Amino-pentyloxy)-3-methyl-phenyl]-1-ethyl-propyl}-2-methyl-phenyl)-1,1,1-trifluoro-2-trifluoromethyl-butan-2-ol, 4-[4-(1-Ethyl-1-{3-methyl-4-[4-(1H-tetrazol-5-yl)-butoxy]-phenyl}-propyl)-2-methyl-phenyl]-1,1,1-trifluoro-2-trifluoromethyl-butan-2-ol, (S)-5-(4-{1-Ethyl-1-[4-(3-hydroxy-3-methyl-but-1-ynyl)-3-methyl-phenyl]-propyl}-2-methyl-phenoxy)-4-hydroxy-pentanoic acid, (R)-5-(4-{1-Ethyl-1-[4-(3-hydroxy-3-methyl-but-1-ynyl)-3-methyl-phenyl]-propyl}-2-methyl-phenoxy)-4-hydroxy-pentanoic acid, (S)-6-(4-{1-Ethyl-1-[4-(3-hydroxy-3-methyl-but-1-ynyl)-3-methyl-phenyl]-propyl}-2-methyl-phenoxy)-5-hydroxy-hexanoic acid, (R)-6-(4-{1-Ethyl-1-[4-(3-hydroxy-3-methyl-but-1-ynyl)-3-methyl-phenyl]-propyl}-2-methyl-phenoxy)-5-hydroxy-hexanoic acid, (S)-3-(4-{1-Ethyl-1-[4-(3-hydroxy-3-methyl-but-1-ynyl)-3-methyl-phenyl]-propyl}-2-methyl-phenoxy)-propane-1,2-diol, 2-(4-{1-Ethyl-1-[4-(3-hydroxy-3-methyl-but-1-ynyl)-3-methyl-phenyl]-propyl}-2-methyl-phenoxymethyl)-propane-1,3-diol, 4-{1-Ethyl-1-[4-(3-hydroxy-3-methyl-but-1-ynyl)-3-methyl-phenyl]-propyl}-2-methyl-phenol, 5-(4-{1-Ethyl-1-[4-(3-hydroxy-3-methyl-but-1-ynyl)-3-methyl-phenyl]-propyl}-2-methyl-phenoxy)-pentanoic acid, 6-(4-{1-Ethyl-1-[4-(3-hydroxy-3-methyl-but-1-ynyl)-3-methyl-phenyl]-propyl}-2-methyl-phenoxy)-hexanoic acid, 3-(4-{1-Ethyl-1-[4-(3-hydroxy-3-methyl-but-1-ynyl)-3-methyl-phenyl]-propyl}-2-methyl-phenyl)-propionic acid, 4-(4-(1-[4-(5-Amino-pentyloxy)-3-methyl-phenyl]-1-ethyl-propyl}-2-methyl-phenyl)-2-methyl-but-3-yn-2-ol, 4-[4-(1-Ethyl-1-{3-methyl-4-[4-(1H-tetrazol-5-yl)-butoxy]-phenyl}-propyl)-2-methyl-phenyl]-2-methyl-but-3-yn-2-ol, (S)-5-(4-{1-Ethyl-1-[4-((E)-3-hydroxy-3-methyl-but-1-enyl)-3-methyl-phenyl]-propyl}-2-methyl-phenoxy)-4-hydroxy-pentanoic acid, (R)-5-(4-{1-Ethyl-1-[4-((E)-3-hydroxy-3-methyl-but-1-enyl)-3-methyl-phenyl]-propyl}-2-methyl-phenoxy)-4-hydroxy-pentanoic acid, (S)-6-(4-{1-Ethyl-1-[4-((E)-3-hydroxy-3-methyl-but-1-enyl)-3-methyl-phenyl]-propyl}-2-methyl-phenoxy)-5-hydroxy-hexanoic acid, (R)-6-(4-{1-Ethyl-1-[4-((E)-3-hydroxy-3-methyl-but-1-enyl)-3-methyl-phenyl]-propyl}-2-methyl-phenoxy)-5-hydroxy-hexanoic acid, (S)-3-(4-{1-Ethyl-1-[4-((E)-3-hydroxy-3-methyl-but-1-enyl)-3-methyl-phenyl]-propyl}-2-methyl-phenoxy)-propane-1,2-diol, 2-(4-{1-Ethyl-1-[4-((E)-3-hydroxy-3-methyl-but-1-enyl)-3-methyl-phenyl]-propyl}-2-methyl-phenoxymethyl)-propane-1,3-diol, 4-{1-Ethyl-1-[4-((E)-3-hydroxy-3-methyl-but-1-enyl)-3-methyl-phenyl]-propyl}-2-methyl-phenol, 5-(4-{1-Ethyl-1-[4-((E)-3-hydroxy-3-methyl-but-1-enyl)-3-methyl-phenyl]-propyl}-2-methyl-phenoxy)-pentanoic acid, 6-(4-{1-Ethyl-1-[4-((E)-3-hydroxy-3-methyl-but-1-enyl)-3-methyl-phenyl]-propyl}-2-methyl-phenoxy)-hexanoic acid, 3-(4-{1-Ethyl-1-[4-((E)-3-hydroxy-3-methyl-but-1-enyl)-3-methyl-phenyl]-propyl}-2-methyl-phenyl)-propionic acid, (E)-4-(4-{1-[4-(5-Amino-pentyloxy)-3-methyl-phenyl]-1-ethyl-propyl}-2-methyl-phenyl)-2-methyl-but-3-en-2-ol, (E)-4-[4-(1-Ethyl-1-{3-methyl-4-[4-(1H-tetrazol-5-yl)-butoxy]-phenyl}-propyl)-2-methyl-phenyl]-2-methyl-but-3-en-2-ol, (S )-5-(4-{1-Ethyl-1-[4-(3-hydroxy-3-methyl-butyl)-3-methyl-phenyl]-propyl}-2-methyl-phenoxy)-4-hydroxy-pentanoic acid, (R)-5-(4-{1-Ethyl-1-[4-(3-hydroxy-3-methyl-butyl)-3-methyl-phenyl]-propyl}-2-methyl-phenoxy)-4-hydroxy-pentanoic acid, (S)-6-(4-{1-Ethyl-1-[4-(3-hydroxy-3-methyl-butyl)-3-methyl-phenyl]-propyl}-2-methyl-phenoxy)-5-hydroxy-hexanoic acid, (R)-6-(4-{1-Ethyl-1-[4-(3-hydroxy-3-methyl-butyl)-3-methyl-phenyl]-propyl}-2-methyl-phenoxy)-5-hydroxy-hexanoic acid, (S)-3-(4-{1-Ethyl-1-[4-(3-hydroxy-3-methyl-butyl)-3-methyl-phenyl]-propyl}-2-methyl-phenoxy)-propane-1,2-diol, 2-(4-{1-Ethyl-1-[4-(3-hydroxy-3-methyl-butyl)-3-methyl-phenyl]-propyl}-2-methyl-phenoxymethyl)-propane-1,3-diol, 4-{1-Ethyl-1-[4-(3-hydroxy-3-methyl-butyl)-3-methyl-phenyl]-propyl}-2-methyl-phenol, 5-(4-{1-Ethyl-1-[4-(3-hydroxy-3-methyl-butyl)-3-methyl-phenyl]-propyl}-2-methyl-phenoxy)-pentanoic acid, 6-(4-{1-Ethyl-1-[4-(3-hydroxy-3-methyl-butyl)-3-methyl-phenyl]-propyl}-2-methyl-phenoxy)-hexanoic acid, 3-(4-{1-Ethyl-1-[4-(3-hydroxy-3-methyl-butyl)-3-methyl-phenyl]-propyl}-2-methyl-phenyl)-propionic acid, 4-(4-{1-[4-(5-Amino-pentyloxy)-3-methyl-phenyl]-1-ethyl-propyl}-2-methyl-phenyl)-2-methyl-butan-2-ol, 4-[4-(1-Ethyl-1-{3-methyl-4-[4-(1H-tetrazol-5-yl)-butoxy]-phenyl}-propyl)-2-methyl-phenyl]-2-methyl-butan-2-ol, (S)-5-(4-{1-Ethyl-1-[4-(1-hydroxy-cyclobutylethynyl)-3-methyl-phenyl]-propyl}-2-methyl-phenoxy)-4-hydroxy-pentanoic acid, (R)-5-(4-{1-Ethyl-1-[4-(1-hydroxy-cyclobutylethynyl)-3-methyl-phenyl]-propyl}-2-methyl-phenoxy)-4-hydroxy-pentanoic acid, (S)-6-(4-{1-Ethyl-1-[4-(1-hydroxy-cyclobutylethynyl)-3-methyl-phenyl]-propyl}-2-methyl-phenoxy)-5-hydroxy-hexanoic acid, (R)-6-(4-{1-Ethyl-1-[4-(1-hydroxy-cyclobutylethynyl)-3-methyl-phenyl]-propyl}-2-methyl-phenoxy)-5-hydroxy-hexanoic acid, (S)-3-(4-{1-Ethyl-1-[4-(1-hydroxy-cyclobutylethynyl)-3-methyl-phenyl]-propyl}-2-methyl-phenoxy)-propane-1,2-diol, 2-(4-{1-Ethyl-1-[4-(1-hydroxy-cyclobutylethynyl)-3-methyl-phenyl]-propyl}-2-methyl-phenoxymethyl)-propane-1,3-diol, 4-{1-Ethyl-1-[4-(1-hydroxy-cyclobutylethynyl)-3-methyl-phenyl]-propyl}-2-methyl-phenol, 5-(4-{1-Ethyl-1-[4-(1-hydroxy-cyclobutylethynyl)-3-methyl-phenyl]-propyl}-2-methyl-phenoxy)-pentanoic acid, 6-(4-{1-Ethyl-1-[4-(1-hydroxy-cyclobutylethynyl)-3-methyl-phenyl]-propyl}-2-methyl-phenoxy)-hexanoic acid, 3-(4-{1-Ethyl-1-[4-(1-hydroxy-cyclobutylethynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenylethynyl)-cyclobutanol, 1-[4-(1-Ethyl-1-{3-methyl-4-[4-(1H-tetrazol-5-yl)-butoxy]-phenyl}-propyl)-2-methyl-phenylethynyl]-cyclobutanol, (S)-5-[4-(1-Ethyl-1-{4-[(E)-2-(1-hydroxy-cyclobutyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclobutyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclobutyl)-vinyl]-3-methyl-phenyl}-propyl)-2-methyl-phenoxy]-5-hydroxy-hexanoic acid, (R)-6-[4-(1-Ethyl-1-{4-[(E)-2-(1-hydroxy-cyclobutyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclobutyl)-vinyl]-3-methyl-phenyl)-propyl)-2-methyl-phenoxy]-propane-1,2-diol, 2-[4-(1-Ethyl-1-{4-[(E)-2-(1-hydroxy-cyclobutyl)-vinyl]-3-methyl-phenyl}-propyl)-2-methyl-phenoxymethyl]-propane-1,3-diol, 4-(1-Ethyl-1-{4-[(E)-2-(1-hydroxy-cyclobutyl)-vinyl]-3-methyl-phenyl}-propyl)-2-methyl-phenol, 5-[4-(1-Ethyl-1-{4-[(E)-2-(1-hydroxy-cyclobutyl)-vinyl]-3-methyl-phenyl}-propyl)-2-methyl-phenoxy]-pentanoic acid, 6-[4-(1-Ethyl-1-{4-[(E)-2-(1-hydroxy-cyclobutyl)-vinyl]-3-methyl-phenyl}-propyl)-2-methyl-phenoxy]-hexanoic acid, 3-[4-(1-Ethyl-1-{4-[(E)-2-(1-hydroxy-cyclobutyl)-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclobutanol, 1-{(E)-2-[4-(1-Ethyl-1-(3-methyl-4-[4-(1H-tetrazol-5-yl)-butoxy]-phenyl}-propyl)-2-methyl-phenyl]-vinyl}-cyclobutanol, (S)-5-[4-(1-Ethyl-1-{4-[2-(1-hydroxy-cyclobutyl)-ethyl]-3-methyl-phenyl}-propyl)-2-methyl-phenoxy]-4-hydroxy-pentanoic acid, (R)-5-[4-(1-Ethyl-1-{4-[2-(1-hydroxy-cyclobutyl)-ethyl]-3-methyl-phenyl}-propyl)-2-methyl-phenoxy]4-hydroxy-pentanoic acid, (S)-6-[4-(1-Ethyl-1-{4-[2-(1-hydroxy-cyclobutyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclobutyl)-ethyl]-3-methyl-phenyl}-propyl)-2-methyl-phenoxy]-5-hydroxy-hexanoic acid, (S)-3-[4-(1-Ethyl-1-{4-[2-(1-hydroxy-cyclobutyl)-ethyl]-3-methyl-phenyl}-propyl)-2-methyl-phenoxy]-propane-1,2-diol, 2-[4-(1-Ethyl-1-{4-[2-(1-hydroxy-cyclobutyl)-ethyl]-3-methyl-phenyl}-propyl)-2-methyl-phenoxymethyl]-propane-1,3-diol, 4-(1-Ethyl-1-{4-[2-(1-hydroxy-cyclobutyl)-ethyl]-3-methyl-phenyl}-propyl)-2-methyl-phenol, 5-[4-(1-Ethyl-1-{4-[2-(1-hydroxy-cyclobutyl)-ethyl]-3-methyl-phenyl)-propyl)-2-methyl-phenoxy]-pentanoic acid, 6-[4-(1-Ethyl-1-{4-[2-(1-hydroxy-cyclobutyl)-ethyl]-3-methyl-phenyl}-propyl)-2-methyl-phenoxy]-hexanoic acid, 3-[4-(1-Ethyl-1-{4-[2-(1-hydroxy-cyclobutyl)-ethyl]-3-methyl-phenyl}-propyl)-2-methyl-phenyl]-propionic acid, 1-[2-(4-1-[4-(5-Amino-pentyloxy)-3-methyl-phenyl]-1-ethyl-propyl}-2-methyl-phenyl)-ethyl]-cyclobutanol, 1-{2-[4-(1-Ethyl-1-{3-methyl-4-[4-(1H-tetrazol-5-yl)-butoxy]-phenyl}-propyl)-2-methyl-phenyl]-ethyl}-cyclobutanol, (S)-5-(4-{1-Ethyl-1-[4-(1-hydroxy-cyclopentylethynyl)-3-methyl-phenyl]-propyl}-2-methyl-phenoxy)-4-hydroxy-pentanoic acid, (R)-5-(4-{1-Ethyl-1-[4-(1-hydroxy-cyclopentylethynyl)-3-methyl-phenyl]-propyl}-2-methyl-phenoxy)-4-hydroxy-pentanoic acid, (S)-6-(4-{1-Ethyl-1-[4-(1-hydroxy-cyclopentylethynyl)-3-methyl-phenyl]-propyl}-2-methyl-phenoxy)-5-hydroxy-hexanoic acid, (R)-6-(4-{1-Ethyl-1-[4-(1-hydroxy-cyclopentylethynyl)-3-methyl-phenyl]-propyl}-2-methyl-phenoxy)-5-hydroxy-hexanoic acid, (S)-3-(4-{1-Ethyl-1-[4-(1-hydroxy-cyclopentylethynyl)-3-methyl-phenyl]-propyl}-2-methyl-phenoxy)-propane-1,2-diol, 2-(4-{1-Ethyl-1-[4-(1-hydroxy-cyclopentylethynyl)-3-methyl-phenyl]-propyl}-2-methyl-phenoxymethyl)-propane-1,3-diol, 4-{1-Ethyl-1-[4-(1-hydroxy-cyclopentylethynyl)-3-methyl-phenyl]-propyl}-2-methyl-phenol, 5-(4-{1-Ethyl-1-[4-(1-hydroxy-cyclopentylethynyl)-3-methyl-phenyl]-propyl}-2-methyl-phenoxy)-pentanoic acid, 6-(4-{1-Ethyl-1-[4-(1-hydroxy-cyclopentylethynyl)-3-methyl-phenyl]-propyl}-2-methyl-phenoxy)-hexanoic acid, 3-(4-{1-Ethyl-1-[4-(1-hydroxy-cyclopentylethynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenylethynyl)-cyclopentanol, 1-[4-(1-Ethyl-1-{3-methyl-4-[4-(1H-tetrazol-5-yl)-butoxy]-phenyl}-propyl)-2-methyl-phenylethynyl]-cyclopentanol, (S)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclopentyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclopentyl)-vinyl]-3-methyl-phenyl}-propyl)-2-methyl-phenoxy]-5-hydroxy-hexanoic acid, (R)-6-[4-(1-Ethyl-1-{4-[(E)-2-(1-hydroxy-cyclopentyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclopentyl)-vinyl]-3-methyl-phenyl}-propyl)-2-methyl-phenoxy]-propane-1,2-diol, 2-[4-(1-Ethyl-1-{4-[(E)-2-(1-hydroxy-cyclopentyl)-vinyl]-3-methyl-phenyl}-propyl)-2-methyl-phenoxymethyl]-propane-1,3-diol, 4-(1-Ethyl-1-{4-[(E)-2-(1-hydroxy-cyclopentyl)-vinyl]-3-methyl-phenyl}-propyl)-2-methyl-phenol, 5-[4-(1-Ethyl-1-{4-[(E)-2-(1-hydroxy-cyclopentyl)-vinyl]-3-methyl-phenyl}-propyl)-2-methyl-phenoxy]-pentanoic acid, 6-[4-(1-Ethyl-1-{4-[(E)-2-(1-hydroxy-cyclopentyl)-vinyl]-3-methyl-phenyl}-propyl)-2-methyl-phenoxy]-hexanoic acid, 3-[4-(1-Ethyl-1-{4-[(E)-2-(1-hydroxy-cyclopentyl)-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclopentanol, 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclopentanol, (S)-5-[4-(1-Ethyl-1-{4-[2-(1-hydroxy-cyclopentyl)-ethyl]-3-methyl-phenyl}-propyl)-2-methyl-phenoxy]4-hydroxy-pentanoic acid, (R)-5-[4-(1-Ethyl-1-{4-[2-(1-hydroxy-cyclopentyl )-ethyl]-3-methyl-phenyl}-propyl)-2-methyl-phenoxy]-4-hydroxy-pentanoic acid, (S)-6-[4-(1-Ethyl-1-{4-[2-(1-hydroxy-cyclopentyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclopentyl)-ethyl]-3-methyl-phenyl}-propyl)-2-methyl-phenoxy]-5-hydroxy-hexanoic acid, (S)-3-[4-(1-Ethyl-1-{4-[2-(1-hydroxy-cyclopentyl)-ethyl]-3-methyl-phenyl}-propyl)-2-methyl-phenoxy]-propane-1,2-diol, 2-[4-(1-Ethyl-1-{4-[2-(1-hydroxy-cyclopentyl)-ethyl]-3-methyl-phenyl}-propyl)-2-methyl-phenoxymethyl]-propane-1,3-diol, 4-(1-Ethyl-1-{4-[2-(1-hydroxy-cyclopentyl)-ethyl]-3-methyl-phenyl}-propyl)-2-methyl-phenol, 5-[4-(1-Ethyl-1-{4-[2-(1-hydroxy-cyclopentyl)-ethyl]-3-methyl-phenyl}-propyl)-2-methyl-phenoxy]-pentanoic acid, 6-[4-(1-Ethyl-1-{4-[2-(1-hydroxy-cyclopentyl)-ethyl]-3-methyl-phenyl}-propyl)-2-methyl-phenoxy]-hexanoic acid, 3-[4-(1-Ethyl-1-{4-[2-(1-hydroxy-cyclopentyl)-ethyl]-3-methyl-phenyl}-propyl)-2-methyl-phenyl]-propionic acid, 1-[2-(4-{1-[4-(5-Amino-pentyloxy)-3-methyl-phenyl]-1-ethyl-propyl}-2-methyl-phenyl)-ethyl]-cyclopentanol, 1-{2-[4-(1-Ethyl-1-{3-methyl-4-[4-(1H-tetrazol-5-yl)-butoxy]-phenyl}-propyl)-2-methyl-phenyl]-ethyl}-cyclopentanol, (S)-5-(4-{1-Ethyl-1-[4-(1-hydroxy-cyclohexylethynyl)-3-methyl-phenyl]-propyl}-2-methyl-phenoxy)-4-hydroxy-pentanoic acid, (R)-5-(4-{1-Ethyl-1-[4-(1-hydroxy-cyclohexylethynyl)-3-methyl-phenyl]-propyl}-2-methyl-phenoxy)-4-hydroxy-pentanoic acid, (S)-6-(4-{1-Ethyl-1-[4-(1-hydroxy-cyclohexylethynyl)-3-methyl-phenyl]-propyl}-2-methyl-phenoxy)-5-hydroxy-hexanoic acid, (R)-6-(4-{1-Ethyl-1-[4-(1-hydroxy-cyclohexylethynyl)-3-methyl-phenyl]-propyl}-2-methyl-phenoxy)-5-hydroxy-hexanoic acid, (S)-3-(4-{1-Ethyl-1-[4-(1-hydroxy-cyclohexylethynyl)-3-methyl-phenyl]-propyl}-2-methyl-phenoxy)-propane-1,2-diol, 2-(4-{1-Ethyl-1-[4-(1-hydroxy-cyclohexylethynyl)-3-methyl-phenyl]-propyl}-2-methyl-phenoxymethyl)-propane-1,3-diol, 4-{1-Ethyl-1-[4-(1-hydroxy-cyclohexylethynyl)-3-methyl-phenyl]-propyl}-2-methyl-phenol, 5-(4-{1-Ethyl-1-[4-(1-hydroxy-cyclohexylethynyl)-3-methyl-phenyl]-propyl}-2-methyl-phenoxy)-pentanoic acid, 6-(4-{1-Ethyl-1-[4-(1-hydroxy-cyclohexylethynyl)-3-methyl-phenyl]-propyl}-2-methyl-phenoxy)-hexanoic acid, 3-(4-{1-Ethyl-1-[4-(1-hydroxy-cyclohexylethynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenylethynyl)-cyclohexanol, 1-[4-(1-Ethyl-1-{3-methyl-4-[4-(1H-tetrazol-5-yl)-butoxy]-phenyl}-propyl)-2-methyl-phenylethynyl]-cyclohexanol, (S)-5-[4-(1-Ethyl-1-{4-[(E )-2-(1-hydroxy-cyclohexyl)-vinyl]-3-methyl-phenyl}-propyl)-2-methyl-phenoxy]-4-hydroxy-pentanoic acid, (R)-5-[4-(1-Ethyl-1-{4-[(E)-2-(1-hydroxy-cyclohexyl)-vinyl]-3-methyl-phenyl}-propyl)-2-methyl-phenoxy]-4-hydroxy-pentanoic acid, (S)-6-[4-(1-Ethyl-1-{4-[(E)-2-(1-hydroxy-cyclohexyl)-vinyl]-3-methyl-phenyl}-propyl )-2-methyl-phenoxy]-5-hydroxy-hexanoic acid, (R)-6-[4-(1-Ethyl-1-{4-[(E)-2-(1-hydroxy-cyclohexyl)-vinyl]-3-methyl-phenyl}-propyl)-2-methyl-phenoxy]-5-hydroxy-hexanoic acid, (S)-3-[4-(1-Ethyl-1-{4-[(E)-2-(1-hydroxy-cyclohexyl)-vinyl]-3-methyl-phenyl}-propyl)-2-methyl-phenoxy]-propane-1,2-diol, 2-[4-(1-Ethyl-1-{4-[(E)-2-(1-hydroxy-cyclohexyl)-vinyl]-3-methyl-phenyl}-propyl)-2-methyl-phenoxymethyl]-propane-1,3-diol, 4-(1-Ethyl-1-{4-[(E)-2-(1-hydroxy-cyclohexyl)-vinyl]-3-methyl-phenyl}-propyl)-2-methyl-phenol, 5-[4-(1-Ethyl-1-{4-[(E)-2-(1-hydroxy-cyclohexyl)-vinyl]-3-methyl-phenyl}-propyl)-2-methyl-phenoxy]-pentanoic acid, 6-[4-(1-Ethyl-1-{4-[(E)-2-(1-hydroxy-cyclohexyl)-vinyl]-3-methyl-phenyl)-propyl}-2-methyl-phenoxy]-hexanoic acid, 3-[4-(1-Ethyl-1-{4-[(E)-2-(1-hydroxy-cyclohexyl )-vinyl]-3-methyl-phenyl}-propyl)-2-methyl-phenyl]-propionic acid, 1-[(E)-2-(4-{1-[4-(5-Amino-pentyloxy)-3-methyl-phenyl]-1-ethyl-propyl}-2-methyl-phenyl)-vinyl]-cyclohexanol, 1-{(E)-2-[4-(1-Ethyl-1-{3-methyl-4-[4-(1H-tetrazol-5-yl)-butoxy]-phenyl}-propyl)-2-methyl-phenyl]-vinyl}-cyclohexanol, (S)-5-[4-(1-Ethyl-1-{4-[2-(1-hydroxy-cyclohexyl)-ethyl]-3-methyl-phenyl}-propyl)-2-methyl-phenoxy]4-hydroxy-pentanoic acid, (R)-5-[4-(1-Ethyl-1-{4-[2-(1-hydroxy-cyclohexyl)-ethyl]-3-methyl-phenyl}-propyl)-2-methyl-phenoxy]4-hydroxy-pentanoic acid, (S)-6-[4-(1-Ethyl-1-{4-[2-(1-hydroxy-cyclohexyl)-ethyl]-3-methyl-phenyl}-propyl)-2-methyl-phenoxy]-5-hydroxy-hexanoic acid, (R)-6-[4-(1-Ethyl-1-{4-[2-(1-hydroxy-cyclohexyl)-ethyl]-3-methyl-phenyl}-propyl)-2-methyl-phenoxy]-5-hydroxy-hexanoic acid, (S)-3-[4-(1-Ethyl-1-{-4-[2-(1-hydroxy-cyclohexyl)-ethyl]-3-methyl-phenyl}-propyl)-2-methyl-phenoxy]-propane-1,2-diol, 2-[4-(1-Ethyl-1-{4-[2-(1-hydroxy-cyclohexyl)-ethyl]-3-methyl-phenyl}-propyl)-2-methyl-phenoxymethyl]-propane-1,3-diol, 4-(1-Ethyl-1-{4-[2-(1-hydroxy-cyclohexyl)-ethyl]-3-methyl-phenyl}-propyl)-2-methyl-phenol, 5-[4-(1-Ethyl-1-{4-[2-(1-hydroxy-cyclohexyl)-ethyl]-3-methyl-phenyl}-propyl)-2-methyl-phenoxy]-pentanoic acid, 6-[4-(1-Ethyl-1-{4-[2-(1-hydroxy-cyclohexyl)-ethyl]-3-methyl-phenyl}-propyl)-2-methyl-phenoxy]-hexanoic acid, 3-[4-(1-Ethyl-1-{4-[2-(1-hydroxy-cyclohexyl)-ethyl]-3-methyl-phenyl}-propyl)-2-methyl-phenyl]-propionic acid, 1-[2-(4-{1-[4-(5-Amino-pentyloxy)-3-methyl-phenyl]-1-ethyl-propyl}-2-methyl-phenyl)-ethyl]-cyclohexanol, 1-{2-[4-(1-Ethyl-1-{3-methyl-4-[4-(1H-tetrazol-5-yl)-butoxy]-phenyl}-propyl)-2-methyl-phenyl]-ethyl}-cyclohexanol, (S)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (R)-5-(4-{1-Ethyl-1-[4-(3-hydroxy-4,4-dimethyl-pent-1-ynyl)-3-methyl-phenyl]-propyl}-2-methyl-phenoxy)-4-hydroxy-pentanoic acid, (S)-6-(4-{1-Ethyl-1-[4-(3-hydroxy4,4-dimethyl-pent-1-ynyl)-3-methyl-phenyl]-propyl}-2-methyl-phenoxy)-5-hydroxy-hexanoic acid, (R)-6-(4-{1-Ethyl-1-[4-(3-hydroxy-4,4-dimethyl-pent-1-ynyl)-3-methyl-phenyl]-propyl}-2-methyl-phenoxy)-5-hydroxy-hexanoic acid, (S)-3-(4-{1-Ethyl-1-[4-(3-hydroxy-4,4-dimethyl-pent-1-ynyl)-3-methyl-phenyl]-propyl}-2-methyl-phenoxy)-propane-1,2-diol, 2-(4-{1-Ethyl-1-[4-(3-hydroxy-4,4-dimethyl-pent-1-ynyl)-3-methyl-phenyl]-propyl}-2-methyl-phenoxymethyl)-propane-1,3-diol, 4-{1-Ethyl-1-[4-(3-hydroxy-4,4-dimethyl-pent-1-ynyl )-3-methyl-phenyl]-propyl}-2-methyl-phenol, 5-(4-{1-Ethyl-1-[4-(3-hydroxy-4,4-dimethyl-pent-1-ynyl)-3-methyl-phenyl]-propyl}-2-methyl-phenoxy)-pentanoic acid, 6-(4-{1-Ethyl-1-[4-(3-hydroxy-4,4-dimethyl-pent-1-ynyl)-3-methyl-phenyl]-propyl}-2-methyl-phenoxy)-hexanoic acid, 3-(4-{1-Ethyl-1-[4-(3-hydroxy-4,4-dimethyl-pent-1-ynyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-4,4-dimethyl-pent-1-yn-3-ol, 1-[4-(1-Ethyl-1-{3-methyl-4-[4-(1H-tetrazol-5-yl)-butoxy]-phenyl}-propyl)-2-methyl-phenyl]-4,4-dimethyl-pent-1-yn-3-ol, (S)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (R)-5-(4-{1-Ethyl-1-[4-((E)-3-hydroxy-4,4-dimethyl-pent-1-enyl)-3-methyl-phenyl]-propyl}-2-methyl-phenoxy)-4-hydroxy-pentanoic acid, (S)-6-(4-{1-Ethyl-1-[4-((E)-3-hydroxy-4,4-dimethyl-pent-1-enyl)-3-methyl-phenyl]-propyl}-2-methyl-phenoxy)-5-hydroxy-hexanoic acid, (R)-6-(4-{1-Ethyl-1-[4-((E)-3-hydroxy-4,4-dimethyl-pent-1-enyl)-3-methyl-phenyl]-propyl}-2-methyl-phenoxy)-5-hydroxy-hexanoic acid, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, 2-(4-{1-Ethyl-1-[4-((E)-3-hydroxy-4,4-dimethyl-pent-1-enyl)-3-methyl-phenyl]-propyl}-2-methyl-phenoxymethyl)-propane-1,3-diol, 4-{1-Ethyl-1-[4-((E)-3-hydroxy-4,4-dimethyl-pent-1-enyl)-3-methyl-phenyl]-propyl}-2-methyl-phenol, 5-(4-{1-Ethyl-1-[4-((E)-3-hydroxy-4,4-dimethyl-pent-1-enyl)-3-methyl-phenyl]-propyl}-2-methyl-phenoxy)-pentanoic acid, 6-(4-{1-Ethyl-1-[4-((E)-3-hydroxy-4,4-dimethyl-pent-1-enyl)-3-methyl-phenyl]-propyl}-2-methyl-phenoxy)-hexanoic acid, 3-(4-{1-Ethyl-1-[4-((E)-3-hydroxy-4,4-dimethyl-pent-1-enyl)-3-methyl-phenyl]-propyl}-2-methyl-phenyl)-propionic acid, (E)-1-(4-{1-[4-(5-Amino-pentyloxy)-3-methyl-phenyl]-1-ethyl-propyl}-2-methyl-phenyl)-4,4-dimethyl-pent-1-en-3-ol, (E)-1-[4-(1-Ethyl-1-{3-methyl-4-[4-(1H-tetrazol-5-yl)-butoxy]-phenyl}-propyl)-2-methyl-phenyl]-4,4-dimethyl-pent-1-en-3-ol, (S)-5-(4-{1-Ethyl-1-[4-(3-hydroxy-4,4-dimethyl-pentyl)-3-methyl-phenyl]-propyl}-2-methyl-phenoxy)-4-hydroxy-pentanoic acid, (R)-5-(4-{1-Ethyl-1-[4-(3-hydroxy-4,4-dimethyl-pentyl)-3-methyl-phenyl]-propyl}-2-methyl-phenoxy)-4-hydroxy-pentanoic acid, (S)-6-(4-{1-Ethyl-1-[4-(3-hydroxy-4,4-dimethyl-pentyl)-3-methyl-phenyl]-propyl}-2-methyl-phenoxy)-5-hydroxy-hexanoic acid, (R)-6-(4-{1-Ethyl-1-[4-(3-hydroxy-4,4-dimethyl-pentyl)-3-methyl-phenyl]-propyl}-2-methyl-phenoxy)-5-hydroxy-hexanoic acid, (S)-3-(4-{1-Ethyl-1-[4-(3-hydroxy-4,4-dimethyl-pentyl)-3-methyl-phenyl]-propyl}-2-methyl-phenoxy)-propane-1,2-diol, 2-(4-{1-Ethyl-1-[4-(3-hydroxy4,4-dimethyl-pentyl)-3-methyl-phenyl]-propyl}-2-methyl-phenoxymethyl)-propane-1,3-diol, 4-{1-Ethyl-1-[4-(3-hydroxy-4,4-dimethyl-pentyl)-3-methyl-phenyl]-propyl}-2-methyl-phenol, 5-(4-{1-Ethyl-1-[4-(3-hydroxy-4,4-dimethyl-pentyl)-3-methyl-phenyl]-propyl}-2-methyl-phenoxy)-pentanoic acid, 6-(4-{1-Ethyl-1-[4-(3-hydroxy4,4-dimethyl-pentyl)-3-methyl-phenyl]-propyl}-2-methyl-phenoxy)-hexanoic acid, 3-(4-{1-Ethyl-1-[4-(3-hydroxy-4,4-dimethyl-pentyl)-3-methyl-phenyl]-propyl}-2-methyl-phenyl)-propionic acid, 1-(4-{1-[4-(5-Amino-pentyloxy)-3-methyl-phenyl]-1-ethyl-propyl}-2-methyl-phenyl)-4,4-dimethyl-pentan-3-ol, 1-[4-(1-Ethyl-1-{3-methyl-4-[4-(1H-tetrazol-5-yl)-butoxy]-phenyl}-propyl)-2-methyl-phenyl]-4,4-dimethyl-pentan-3-ol, 4-(4-{1-Ethyl-1-[4-(3-hydroxy-4,4-dimethyl-pentyl)-3-methyl-phenyl]-propyl}-2-methyl-phenyl)-butane-1,2-diol, (S)-3-(4-{1-Ethyl-1-[4-((R)-3-hydroxy-4,4-dimethyl-pentyl)-3-methyl-phenyl]-propyl}-2-methyl-phenoxy)-propane-1,2-diol, (R)-3-(4-{1-Ethyl-1-[4-((R)-3-hydroxy-4,4-dimethyl-pentyl)-3-methyl-phenyl]-propyl}-2-methyl-phenoxy)-propane-1,2-diol, (S)-3-(4-{1-Ethyl-1-[4-((S)-3-hydroxy-4,4-dimethyl-pentyl)-3-methyl-phenyl]-propyl}-2-methyl-phenoxy)-propane-1,2-diol, (R)-3-(4-{1-Ethyl-1-[4-((S)-3-hydroxy4,4-dimethyl-pentyl)-3-methyl-phenyl]-propyl}-2-methyl-phenoxy)-propane-1,2-diol, 2-(4-{1-Ethyl-1-[4-(3-hydroxy-4,4-dimethyl-pentyl)-3-methyl-phenyl]-propyl}-2-methyl-phenoxymethyl)-propane-1,3-diol, 3-(4-{1-Ethyl-1-[4-(3-hydroxy-4,4-dimethyl-pentyl)-3-methyl-phenyl]-propyl}-2-methyl-phenoxy)-propane-1,2-diol, (S)-5-(4-{1-Ethyl-1-[4-(3-hydroxy-4,4-dimethyl-pentyl)-3-methyl-phenyl]-propyl}-2-methyl-phenoxy)-pentane-1,4-diol, 1-(4-{1-Ethyl-1-[4-(3-hydroxy-4,4-dimethyl-pentyl)-3-methyl-phenyl]-propyl}-2-methyl-phenyl)-4,4-dimethyl-pentan-3-ol, 1-(4-{1-Ethyl-1-[4-(3-hydroxy-propyl)-3-methyl-phenyl]-propyl}-2-methyl-phenyl)-4,4-dimethyl-pentan-3-ol, (S)-3-(4-{1-Ethyl-1-[4-(3-hydroxy-4,4-dimethyl-pentyl)-3-methyl-phenyl]-propyl}-2-methyl-phenoxy)-propane-1,2-diol, (R)-3-(4-{1-Ethyl-1-[4-(3-hydroxy-4,4-dimethyl-pentyl)-3-methyl-phenyl]-propyl}-2-methyl-phenoxy)-propane-1,2-diol, 4-(4-{1-Ethyl-1-[4-(2-hydroxy-3,3-dimethyl-butoxy)-3-methyl-phenyl]-propyl}-2-methyl-phenyl)-butane-1,2-diol, 1-(4-{1-Ethyl-1-[4-(3-hydroxy-propyl)-3-methyl-phenyl]-propyl}-2-methyl-phenoxy)-3,3-dimethyl-butan-2-ol, 3-(4-{1-Ethyl-1-[4-(2-hydroxy-3,3-dimethyl-butoxy)-3-methyl-phenyl]-propyl}-2-methyl-phenyl)-propane-1,2-diol, 3-(4-{1-Ethyl-1-[4-(2-hydroxy-3,3-dimethyl-butoxy)-3-methyl-phenyl]-propyl}-2-methyl-phenyl)-butane-1,2-diol, 1-(4-{1-Ethyl-1-[4-(2-hydroxy-3,3-dimethyl-butoxy)-3-methyl-phenyl]-propyl}-2-methyl-phenyl)-propane-1,2-diol, 1-(4-{1-Ethyl-1-[4-(3-hydroxy-butyl)-3-methyl-phenyl]-propyl}-2-methyl-phenoxy)-3,3-dimethyl-butan-2-ol, (E)-4-(4-{1-Ethyl-1-[4-(2-hydroxy-3,3-dimethyl-butoxy)-3-methyl-phenyl]-propyl}-2-methyl-phenyl)-but-3-en-2-ol, 3-(4-{1-Ethyl-1-[4-(2-hydroxy-3,3-dimethyl-butoxy)-3-methyl-phenyl]-propyl}-2-methyl-phenyl)-butan-1-ol, 1-(4-{1-[4-(2,3-Dihydroxy-propyl)-3-methyl-phenyl]-1-ethyl-propyl}-2-methyl-phenoxy)-3,3-dimethyl-butan-2-one, 1-(4-{1-[4-(2,3-Dihydroxy-1-methyl-propyl)-3-methyl-phenyl]-1-ethyl-propyl}-2-methyl-phenoxy)-3,3-dimethyl-butan-2-one, 1-(4-{1-Ethyl-1-[4-(3-hydroxy-butyl)-3-methyl-phenyl]-propyl}-2-methyl-phenoxy)-3,3-dimethyl-butan-2-one, 3-(4-{1-Ethyl-1-[4-(2-hydroxy-3,3-dimethyl-butyl)-3-methyl-phenyl]-propyl}-2-methyl-phenoxy)-propane-1,2-diol, (E)-1-(4-{1-[4-((S)-2,3-Dihydroxy-propoxy)-3-methyl-phenyl]-1-ethyl-propyl}-2-methyl-phenyl)-4,4-dimethyl-pent-1-en-3-one, (E)-1-(4-{1-[4-((R)-2,3-Dihydroxy-propoxy)-3-methyl-phenyl]-1-ethyl-propyl}-2-methyl-phenyl)-4,4-dimethyl-pent-1-en-3-one, (S)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, (R)-3-(4-{1-Ethyl-1-[4-((E)-3-hydroxy-4,4-dimethyl-pent-1-enyl)-3-methyl-phenyl]-propyl}-2-methyl-phenoxy)-propane-1,2-diol, 1-(4-{1-[4-((R)-2,3-Dihydroxy-propoxy)-3-methyl-phenyl]-1-ethyl-propyl}-2-methyl-phenyl)-4,4-dimethyl-pentan-3-one.

70. The compound according to claim 1 which is (S)-5-(4-{l-Ethyl-1-[4-((E)-3-ethyl-3-hydroxy-pent-1-enyl)-3-methyl-phenyl]-propyl}-2-methyl-phenoxy)-4-hydroxy-pentanoic acid.

71. The compound according to claim 1 which is (S)-5-(4-{1-Ethyl-1-[3-methyl-4-(4,4,4-trifluoro-3-hydroxy-3-trifluoromethyl-but-1-ynyl)-phenyl]-propyl}-2-methyl-phenoxy)-4-hydroxy-pentanoic acid.

72. A pharmaceutical composition comprising the compound according to claim 1.

73. A pharmaceutical composition comprising a therapeutically effective amount of the compound according to claim 1 in combination with a pharmaceutically acceptable carrier.

74. A method for modulating the activity of the vitamin D receptor in a mammal comprising administering an effective amount of the compound according to claim 1.

75. A method of treating a condition associated with the vitamin D receptor in a mammal comprising administering an affective amount of the compound according to claim 1.

76. A method of treating a condition comprising administering an affective amount of the compound according to claim 1, wherein the condition is selected from the group consisting of abscess, acne, adhesion, alopecia, Alzheimer's disease, benign prostatic hyperplasia, bone fracture healing, cancer, autoimmune induced diabetes, host-graft rejection, insufficient sebum secretion, insufficient dermal firmness, humoral hypercalcemia, insufficient dermal hydration, leukemia, lupus, multiple sclerosis, osteomalacia, osteoporosis, psoriatic arthritis, psoriasis, renal failure, renal osteodystrophy, rheumatoid arthritis, scleroderma, secondary hyperparathyroidism, systemic lupus erythematbsus, and wrinkles, cornea wound, cornea healing, retinopathy, sway, muscle weakness, fall, chronic glomerulonephritis, lupus nephritis, diabetic nephropathy, hypocalcemia, hypoparathyroidism, rickets, osteoarthritis.

77. The method according to claim 76 wherein said condition is selected from the group consisting of benign prostatic hyperplasia, cancer, osteoporosis, psoriasis, secondary hyperparathyroidism, chronic glomerulonephritis, lupus nephritis, diabetic nephropathy, sway, muscle weakness, fall, rheumatoid arthritis, osteoarthritis.

78. The method according to claim 76 wherein said condition is benign prostatic hyperplasia.

79. The method according to claim 76 wherein said condition is cancer.

80. The method according to claim 76 wherein said condition is osteoporosis.

81. The method according to claim 76 wherein said condition is psoriasis.

82. The method according to claim 76 wherein said condition is secondary hyperparathyroidism.

83. The method according to claim 76 wherein said condition is chronic glomerulonephritis.

84. The method according to claim 76 wherein said condition is lupus nephritis.

85. The method according to claim 73 wherein said condition is diabetic nephropathy.

86. The compound according to claim 1 wherein Y is a substituent represented by following formula: 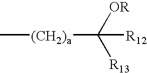
and R is a protecting group for a hydroxyl group.

87. The compound according to claim 86 wherein
X is an optionally substituted methylene, an optionally substituted ethylene, an optionally substituted vinylene or an ethynylene;
R is a protecting group for a hydroxyl group selected from the group consisting of a methoxymethyl group, a methylthiomethyl group, a (phenyldimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group, a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-trifluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyldimethylsilyl group, a t-buthyldiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl -group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group;
R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;
W is a substituent represented by following formula: 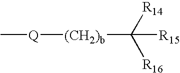
Q is —O—, a methylene, an ethylene, a vinylene, an ethynylene, —(CH2)ₖ-C(═O)NH— or —O—(CH2)ₖ-C(═O)NH—;
R14 is a hydrogen atom, a hydroxyl group, an optionally substituted carboxyl group, an optionally substituted carbamoyl group, an optionally substituted C1-6 alkyl group, an optionally substituted C3-C12 cycloalkyl group, an optionally substituted C6-C12 aryl group, an optionally substituted (C6-C12)aryl-(C14)alkyl group, —OR17 or —N(R18)R19;
R15 and R16 are each independently selected from the group consisting of a hydrogen atom, an optionally substituted C1-6 alkyl group, an optionally substituted C3-C12 cycloalkyl group, an optionally substituted C6-C12 aryl group, an optionally substituted (C6-C12)aryl-(C1-4)alkyl group, an optionally substituted 3-12 membered heterocyclic group selected from the group consisting of an aziridine group, an azetidine group, an oxetane group, a pyrrolidine group, a tetrahydrofuran group, a pyrrole group, a furan group, a thiophene group, a pyrazole group, an isoxazole group, an isothiazole group, an imidazole group, an oxazole group, a thiazolegroup, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a piperidine group, a pyridine group, a pyridazine group, a pyrimidine group, a pyrazine group, a tetrahydropyran group, a pyran group, a thiopyran group, an indole group, a benzofuran group, a benzothiophene group, an indazole group, a benzisoxazole group, a benzisothiazole group, a benzimidazole group, a benzoxazole group, a benzothiazole gorup, a quinoline group, an isoquinoline group, a cinnoline group, a phthalazine group, a quinazoline group, a quinoxaline group, or R15 and R16 may together form ═O;
R17 is an optionally substituted C1-6 alkyl group or an optionally substituted C3-C6 cycloalkyl group;
R18 and R19 are each independently selected from a hydrogen atom, an optionally substituted C1-6 alkyl group or an optionally substituted C3-C6 cycloalkyl group;
or one of (R15 and R17), (R16 and R17), (R15 and R18), (R16 and R18), (R15 and R19) or (R16 and R19) may together form a 3-12 membered cyclic ring which is selected from a lactam ring or a lactone ring;
R1 is a C1-6 alkyl group;
R2 is a C1-6 alkyl group;
R3 is a hydrogen atom or a C1-6 alkyl group;
R4 is a halogen atom or a C1-6 alkyl group;
R5 is a hydrogen atom;
R6 is a halogen atom or a C1-6 alkyl group.

88. The compound according to claim 87 wherein
X is an ethylene, a vinylene, or an ethynylene;
R is a protecting group for a hydroxyl group selected from the group consisting of a methoxymethyl group, a 2-(trimethylsilyl)ethoxymethyl group, a tetrahydropyranyl group, a benzyl group, a p-methoxybenzyl group, a trimethylsilyl group, a triethylsilyl group, a t-butyidimethylsilyl group, a t-buthyldiphenylsilyl group, an acetyl group, a pivaloyl group, a benzoyl group, a methanesulfonyl group, a tosyl group, a trifluoromethanesulfonyl group;
R12 and R13 are each independently selected from the group consisting of a hydrogen atom, a C1-8 alkyl group which may be substituted with a halogen atom(s) a C3-8 cycloalkyl group which may be substituted with a C1-4 alkyl group, or R12 and R13 are together form a C3-8 cycloalkyl group which may be substituted with a C1-4 alkyl group;
W is a substituent represented by following formula: 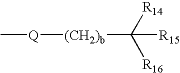
Q is —O—, a methylene, an ethylene, a vinylene, an ethynylene, —(CH2)ₖ-C(═O)NH— or —O—(CH2)ₖ-C(═O)NH—;
b is an integer of 0 to 5;
R14 is a hydrogen atom, a hydroxyl group, a carboxyl group which may be substituted with a C1-4 alkyl group, a carbamoyl group which may be substituted with a C1-4 alkyl group, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, OR17 or —N(R18)R19;
R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group which may be substituted with a C1-4 alkyl group, a phenyl group which may be substituted with a C1-4 alkyl group, a hydroxyl group, a carboxyl group, a carbamoyl group or an amino group, a benzyl group which may be substituted with a C1-4 alkyl group, a hydroxyl group, a carboxyl group, a carbamoyl group or an amino group, a 3-8 membered heterocyclic group which may be substituted with a C1-4 alkyl group, a hydroxyl group, a carboxyl group, a carbamoyl group or an amino group, wherein said heterocyclic group selected from the group consisting of an oxetane group, a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a tetrazole group, a pyridine group, a pyridazine group, a pyrimidine group, a pyrazine group, a tetrahydropyran group, a pyran group, a thiopyran group, or R15 and R16 may together form ═O;
and at least one of R14, R15 or R16 is a hydrogen atom;
R17 is a C1-4 alkyl group;
R18 and R19 are each independently selected from a hydrogen atom or a C1-4 alkyl group;
or one of (R15 and R17), (R16 and R17), (R15 and R18), (R16 and R18), (R15 and R19) or (R16 and R19) may together form a 3-12 membered lactone ring.

89. The compound according to claim 88 wherein
R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;
W is a substituent represented by following formula: 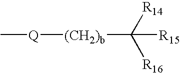
Q is —O—, —(CH2)ₖ-C(═O)NH— or —O—(CH2)ₖ-C(═O)NH—;
b is 0, 1 or 2;
k is 1;
R14 is a hydrogen atom, a hydroxyl group, a carboxyl group, a carbamoyl group, a C1-6 alkyl group which may be substituted with a hydroxyl group, a carboxyl group, a carbamoyl group or an amide group, a C3-C8 cycloalkyl group, a phenyl group which may be substituted with a hydroxyl group or a carboxyl group, a benzyl group which may be substituted with a hydroxyl group or a carboxyl group, —OR17 or —N(R18)R19;
R15 and R16 are each independently selected from the group consisting of a hydrogen atom, a C1-6 alkyl group which may be substituted with a hydroxyl group, a carboxyl group, a carbamoyl group or an amino group, a C3-C8 cycloalkyl group, a phenyl group which may be substituted with a hydroxyl group or a carboxyl group, a benzyl group which may be substituted with a hydroxyl group or a-carboxyl group, a 3-8 membered heterocyclic group selected from the group consisting of a tetrahydrofuran group, a pyrrole group, a furan group, a thiophene group, a pyrazole group, an isoxazole group, an isothiazole group, an oxazole group, a thiazole group, a 1,2,5-oxadiazole group, a 1,3,4-oxadiazole group, a 1,3,4-thiadiazole group, a 1,2,4-oxadiazole group, a 1,2,4-thiadiazole group, a pyridine group, a tetrahydropyran group;
and at least one of R14, R15 or R16 is a hydrogen atom;
R18 is a hydrogen atom;
R19 is a hydrogen atom;
or one of (R15 and R17), (R16 and R17), (R15 and R18), (R16 and R18), (R15 and R19) or (R16 and R19) may together form a 3-12 membered lactone ring;
R1 is a C1-4 alkyl group;
R2 is a C1-4 alkyl group;
R3 is a hydrogen atom or a C1-4 alkyl group;
R4 is a halogen atom or a C1-4 alkyl group;
R5 is a hydrogen atom;
R6 is a halogen atom or a C1-4 alkyl group.

90. The compound according to claim 89 wherein
R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),
or R12 and R13 are together form a C3-10 cycloalkyl group;
Q is —O— or —O—(CH2)ₖ-C(═O)NH—;
R1 is an ethyl group;
R2 is an ethyl group;
R3 is a hydrogen atom or a methyl group;
R4 is a chlorine atom or a methyl group;
R6 is a chlorine atom, a methyl group, an ethyl group, a propyl group or an isopropyl group.

91. The compound according to claim 89 wherein
R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),
or R12 and R13 are together form a C3-10 cycloalkyl group;
Q is a methylene, an ethylene, an ethynylene or —(CH2)ₖ-C(═O)NH—;
R1 is an ethyl group;
R2 is an ethyl group;
R3 is a hydrogen atom;
R4 is a chlorine atom or a methyl group;
R6 is a chlorine atom or a methyl group.

92. The compound according to claim 90 wherein R3 is a hydrogen atom.

93. The compound according to claim 90 wherein R4 is a chlorine atom.

94. The compound according to claim 90 wherein R6 is a chlorine atom.

95. The compound according to claim 90 wherein R6 is a methyl group.

96. The compound according to claim 90 wherein X is an ethylene.

97. The compound according to claim 90 wherein X is a vinylene.

98. The compound according to claim 90 wherein X is an ethynylene.

99. The compound according to claim 90 wherein one of R12 and R13 is a hydrogen atom and the other is a C1-6 alkyl group.

100. The compound according to claim 99 wherein one of R12 and R13 is a hydrogen atom and the other is a tert-butyl group.

101. The compound according to claim 90 wherein both of R12 and R13 are same and a C1-6 alkyl group which may be substituted with a halogen atom(s).

102. The compound according to claim 101 wherein R12 is a trifluoromethyl group and R13 is a trifluoromethyl group.

103. The compound according to claim 90 wherein Q is —O—.

104. The compound according to claim 91 wherein Q is an ethylene.

105. The compound according to claim 91 wherein Q is an ethynylene.

106. The compound according to claim 90 wherein at least one of R14, R15 or R16 is a substituent which have a hydroxyl group, a carboxyl group, a carbamoyl group or an amino group.

107. The compound according to claim 90 wherein R14 is a hydroxyl group.

108. The compound according to claim 90 wherein one of R15 and R16 is a hydrogen atom and the other is a C1-6 alkyl group substituted with a carboxyl group.

109. The compound according to claim 90 wherein one of (R15 and R17), (R16 and R17), (R15 and R18), (R16 and R18), (R15 and R19) or (R16 and R19) together form a 3-12 membered lactone ring.

110. The compound according to claim 1 wherein W is a hydroxyl group, a carboxyl group or a trifluoromethanesulfonyloxy group.

111. The compound according to claim 110 wherein
X is an optionally substituted methylene, an optionally substituted ethylene, an optionally substituted vinylene or an ethynylene;
Y is COOR8, CON(R9)R10, S(O)mR11 or a substituent represented by following formula: 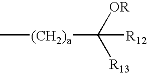
R is a hydrogen atom or a protecting group for a hydroxyl group selected from the group consisting of a methoxymethyl group, a methylthiomethyl group, a (phenyidimethylsilyl)methoxymethyl group, a benzyloxymethyl group, a p-methoxybenzyloxymethyl group, a p-nitrobenzyloxymethyl group, an o-nitrobenzyloxymethyl group, a (4-methoxyphenoxy)methyl group, a guaiacolmethyl group, a t-butoxymethyl group, a 4-pentenyloxymethyl group, a siloxymethyl group, a 2-methoxyethoxymethyl group, 2,2,2-trichloroethoxymethyl group, a bis(2-chloroethoxy)methyl group, a 2-(trimethylsilyl)ethoxymethyl group, a menthoxymethyl group, a tetrahydropyranyl group, a 3-bromotetrahydropyranyl group, a tertahydrothiopyranyl group, a 1-methoxycyclohexyl group, 4-methoxytetrahydrothiopyranyl, a tetrahydrofuranyl group, a tetrahydrothiofuranyl group, a 1-ethoxyethyl group, a 1-(2-chloroethoxy)ethyl group, a 1-[2-(trimethylsilyl)ethoxy]ethyl group, a 1-methyl-1-methoxyethyl group, a 1-methyl-1-benzyloxyethyl group, a 1-methyl-1-benzyloxy-2-fluoroethyl group, a 1-methyl-1-phenoxyethyl group, a 2,2,2-trichloroethyl group, a 1,1-dianisyl-2,2,2-trichloroethyl group, a 2-trimethylsilylethyl group, a 2-(benzylthio)ethyl group, a 2-(phenylselenyl)ethyl group, a t-butyl group, an allyl group, a propargyl group, a p-chlorophenyl group, a p-methoxyphenyl group, a p-nitrophenyl group, a 2,4-dinitrophenyl group, a benzyl group, a p-methoxybenzyl group, a 3,4-dimethoxybenzyl group, an o-nitrophenyl group, a p-nitrophenyl group, a p-halobenzyl group, a 2,6-dichlorobenzyl group, a p-cyanobenzyl group, a p-phenylbenzyl group, a 2,6-difluorobenzyl group, a p-acylaminobenzyl group, a 2-trifluoromethyl benzyl group, a 2-picolyl group, a 4-picolyl group, a 2-quinolinylmethyl group, a triphenylmethyl group, a trimethylsilyl group, a triethylsilyl group, a triisopropylsilyl group, a dimethylisopropylsilyl group, a diethylisopropylsilyl group, a t-butyidimethylsilyl group, a t-buthyldiphenylsilyl group, a tribenzylsilyl group, a triphenylsilyl group, a diphenylmethylsilyl group, a di-t-buthylmethylsilyl group, tris(trimethylsilyl)silyl group, a formyl group, a benzoylformyl group, an acetyl group, a chloroacetyl group, a dichloroacetyl group, a trichloroacetyl group, a methoxyacetyl group, a pivaloyl group, a benzoyl group, a 2,4,6-trimethylbenzoyl group, a methylcarbonyloxy group, a methoxymethylcarbonyloxy group, an ethylcarbonyloxy group, an isobutylcarbonyloxy group, a vinylcarbonyloxy group, an allylcarbonyloxy group, a benzylcarbonyloxy group, a p-methoxybenzylcarbonyloxy group, an allylsulfonyl group, a methanesulfonyl group, a benzylsulfonyl group, a tosyl group, a trifluoromethanesulfonyl group;
R12 and R13 are each independently selected from the group consisting of a hydrogen atom, an optionally substituted C1-10 alkyl group, an optionally substituted C3-10 cycloalkyl group, an optionally substituted C1-10 alkenyl group, an optionally substituted C1-10 alkynyl group, or R12 and R13 may together form an optionally substituted C3-10 cycloalkyl group;
R1 is a C1-6 alkyl group;
R2 is a C1-6 alkyl group;
R3 is a hydrogen atom or a C1-6 alkyl group;
R4 is a halogen atom or a C1-6 alkyl group;
R5 is a hydrogen atom;
R6 is a halogen atom or a C1-6 alkyl group.

112. The compound according to claim 111 wherein
X is an ethylene, a vinylene, or an ethynylene;
Y is a substituent represented by following formula: 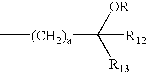
R is a hydrogen atom or a protecting group for a hydroxyl group selected from the group consisting of a methoxymethyl group, a 2-(trimethylsilyl)ethoxymethyl group, a tetrahydropyranyl group, a benzyl group, a p-methoxybenzyl group, a trimethylsilyl group, a triethylsilyl group, a t-butyldimethylsilyl group, a t-buthyldiphenylsilyl group, an acetyl group, a pivaloyl group, a benzoyl group, a methanesulfonyl group, a tosyl group, a trifluoromethanesulfonyl group;
R12 and R13 are each independently selected from the group consisting of a hydrogen atom, a C1-8 alkyl group which may be substituted with a halogen atom(s), a C3-8 cycloalkyl group which may be substituted with a C1-4 alkyl group, or R12 and R13 are together form a C3-8 cycloalkyl group which may be substituted with a C1-4 alkyl group.

113. The compound according to claim 112 wherein
R12 and R13 are each independently selected from the group consisting of a hydrogen atom, a C1-6 alkyl group which may be substituted with a halogen atom(s), a C3-8 cycloalkyl group which may be substituted with a C1-4 alkyl group, or R12 and R13 are together form a C3-8 cycloalkyl group;
R1 is a C1-4 alkyl group;
R2is a C1-4 alkyl group;
R3 is a hydrogen atom or a C1-4 alkyl group;
R4 is a halogen atom or a C1-4 alkyl group;
R5 is a hydrogen atom;
R6 is a halogen atom or a C1-4 alkyl group.

114. The compound according to claim 113 wherein
R12 and R13 are selected from the group consisting of
one of R12 and R13 is a hydrogen atom and the other is a C1-6 alkyl group,
one of R12 and R13 is a hydrogen atom and the other is a C3-8 cycloalkyl group which may be substituted with a C1-4 alkyl group,
both of R12 and R13 are same and a C1-6 alkyl group which may be substituted with a halogen atom(s),
or R12 and R13 are together form a C3-10 cycloalkyl group;
R1 is an ethyl group;
R2 is an ethyl group;
R3 is a hydrogen atom or a methyl group;
R4 is a chlorine atom or a methyl group;
R6 is a chlorine atom, a methyl group, an ethyl group, a propyl group or an isopropyl group.

115. The compound according to claim 114 wherein W is a carboxyl group.

116. The compound according to claim 115 which is 4-{1-Ethyl-1-[4-((E)-3-ethyl-3-hydroxy-pent-1-enyl)-3-methyl-phenyl]-propyl}-2-methyl-benzoic acid.

117. The compound according to claim 114 wherein R3 is a hydrogen atom.

118. The compound according to claim 114 wherein R4 is a chlorine atom.

119. The compound according to claim 114 wherein R6 is a chlorine atom.

120. The compound according to claim 114 wherein R6 is a methyl group.

121. The compound according to claim 114 wherein X is an ethylene.

122. The compound according to claim 114 wherein X is a vinylene.

123. The compound according to claim 114 wherein X is an ethynylene.

124. The compound according to claim 114 wherein one of R12 and R13 is a hydrogen atom and the other is a C1-6 alkyl group.

125. The compound according to claim 124 wherein one of R12 and R13 is a hydrogen atom and the other is a tert-butyl group.

126. The compound according to claim 114 wherein both of R12 and R13 are same and a C1-6 alkyl group which may be substituted with a halogen atom(s).

127. The compound according to claim 126 wherein R12 is a trifluoromethyl group and R13 is a trifluoromethyl group.

128. The compound according to claim 91 wherein R3 is a hydrogen atom.

129. The compound according to claim 91 wherein R4 is a chlorine atom.

130. The compound according to claim 91 wherein R6 is a chlorine atom.

131. The compound according to claim 91 wherein R6 is a methyl group.

132. The compound according to claim 91 wherein X is an ethylene.

133. The compound according to claim 91 wherein X is a vinylene.

134. The compound according to claim 91 wherein X is an ethynylene.

135. The compound according to claim 91 wherein one of R12 and R13 is a hydrogen atom and the other is a C1-6 alkyl group.

136. The compound according to claim 135 wherein one of R12 and R13 is a hydrogen atom and the other is a tert-butyl group.

137. The compound according to claim 91 wherein both of R12 and R13 are same and a C1-6 alkyl group which may be substituted with a halogen atom(s).

138. The compound according to claim 137 wherein R12 is a trifluoromethyl group and R13 is a trifluoromethyl group.

139. The compound according to claim 91 wherein at least one of R14, R15 or R16 is a substituent which have a hydroxyl group, a carboxyl group, a carbamoyl group or an amino group.

140. The compound according to claim 91 wherein R14 is a hydroxyl group.

141. The compound according to claim 91 wherein one of R15 and R16 is a hydrogen atom and the other is a C1-6 alkyl group substituted with a carboxyl group.

142. The compound according to claim 91 wherein one of (R15 and R17), (R16 and R17), (R15 and R18), (R16 and R18), (R15 and R19) or (R16 and R19) together form a 3-12 membered lactone ring.

143. The compound according to claim 115 wherein R3 is a hydrogen atom.

144. The compound according to claim 115 wherein R4 is a chlorine atom.

145. The compound according to claim 115 wherein R6 is a chlorine atom.

146. The compound according to claim 115 wherein R6 is a methyl group.

147. The compound according to claim 115 wherein X is an ethylene.

148. The compound according to claim 115 wherein X is a vinylene.

149. The compound according to claim 115 wherein X is an ethynylene.

150. The compound according to claim 1 15 wherein one of R12 and R13 is a hydrogen atom and the other is a C1-6 alkyl group.

151. The compound according to claim 150 wherein one of R12 and R13 is a hydrogen atom and the other is a tert-butyl group.

152. The compound according to claim 115 wherein both of R12 and R13 are same and a C1-6 alkyl group which may be substituted with a halogen atom(s).

153. The compound according to claim 152 wherein R12 is a trifluoromethyl group and R13 is a trifluoromethyl group.
